(19) United States
(12) Patent Application Publication
(10) Pub. No.: US 2023/0058189 A1
(43) Pub. Date: Feb. 23, 2023

(54) QUINOLIN-4-ONE AND 4(1H)-CINNOLINONE COMPOUNDS AND METHODS OF USING SAME

(71) Applicant: Frequency Therapeutics, Inc., Lexington, MA (US)

(72) Inventors: Bradley TAIT, North Andover, MA (US); Megan S. HARRISON, Middletown, CT (US); Will MCLEAN, North Haven, CT (US)

(73) Assignee: Frequency Therapeutics, Inc., Lexington, MA (US)

(21) Appl. No.: 17/429,091

(22) PCT Filed: Feb. 7, 2020
(86) PCT No.: PCT/US2020/017356

§ 371 Date: Aug. 6, 2021

Related U.S. Application Data
(60) Provisional application No. 62/803,346, filed on Feb. 8, 2019.

(51) Int. Cl. C07D 215/56; C07D 237/28; A61P 27/16; C07D 401/04

(52) U.S. Cl. CPC C07D 215/56; C07D 237/28; A61P 27/16; C07D 401/04

(57) ABSTRACT

The present disclosure relates to quinolin-4-one and 4(1H)-cinnolinone compounds and methods of using them to induce self-renewal of stem/progenitor supporting cells, including inducing the stem/progenitor cells to proliferate while maintaining, in the daughter cells, the capacity to differentiate into tissue cells.

### RELATED APPLICATIONS

This application claims priority to, and the benefit of, U.S. Provisional Application No. 62/803,346, filed Feb. 8, 2019, the entire contents of which is incorporated herein by reference.

### FIELD OF THE DISCLOSURE

The present disclosure relates to quinolin-4-one and 4(1H)-cinnolinone compounds and methods of using them to induce self-renewal of stem/progenitor supporting cells, including inducing the stem/progenitor cells to proliferate while maintaining, in the daughter cells, the capacity to differentiate into tissue cells.

### BACKGROUND OF THE DISCLOSURE

Stem cells exhibit an extraordinary ability to generate multiple cell types in the body. Besides embryonic stem cells, tissue specific stem cells serve a critical role during development as well as in homeostasis and injury repair in the adult. Stem cells renew themselves through proliferation as well as generate tissue specific cell types through differentiation. The characteristics of different stem cells vary from tissue to tissue, and are determined by their intrinsic genetic and epigenetic status. However, the balance between self-renewal and differentiation of different stem cells are all stringently controlled. Uncontrolled self-renewal may lead to overgrowth of stem cells and possibly tumor formation, while uncontrolled differentiation may exhaust the stem cell pool, leading to an impaired ability to sustain tissue homeostasis. Thus, stem cells, continuously sense their environment and appropriately respond with proliferation, differentiation or apoptosis. It would be advantageous to drive regeneration by controlling the timing and extent of stem cell proliferation and differentiation. Controlling the proliferation with small molecules that are cleared over time would allow for control of the timing and extent of stem cell proliferation and differentiation. Remarkably, tissue stem cells from different tissues share a limited number of signaling pathways for the regulations of their self-renewal and differentiation, albeit in a very context dependent manner. Some of these pathways include those where the FOXO-1, GSK3 α/β, and LSD-1 proteins serve regulatory roles.

Lgr5 is expressed across a diverse range of tissues and has been identified as a biomarker of adult stem cells in a variety of tissues such as the gut epithelia (Barker et al. 2007), kidney, hair follicle, and stomach (Barker et al, 2010; Haegebarth & Clevers, 2009). For example, it was first published in 2011, that mammalian inner ear hair cells are derived from LGR5⁺ cells (Chai et al, 2011, Shi et al. 2012). Lgr5 is a known component of the Wnt/beta-catenin pathway, which has been shown to play major roles in differentiation, proliferation, and inducing stem cell characteristics (Barker et al. 2007).

Permanent damage to the hair cells of the inner ear results in sensorineural hearing loss, leading to communication difficulties in a large percentage of the population. Hair cells are the receptor cells that transduce the acoustic stimulus. Regeneration of hair cells would provide an avenue for the treatment of a condition that currently has no therapies other than prosthetic devices. Although hair cells do not spontaneously regenerate in the mammalian cochlea, new hair cells in lower vertebrates are generated from epithelial cells, called supporting cells, that surround hair cells.

Prior work has focused on transdifferentiation of supporting cells into hair cells through activation or forced expression of genes that lead to hair cell formation, with a particular focus on mechanisms to enhance expression of Atoh1 (Bermingham et al., 1999; Zheng and Gao, 2000; Izumikawa et al., 2005; Mizutari et al., 2013). Interestingly, cells transduced with Atoh1 vectors have been shown to acquire vestibular phenotypes (Kawamoto et al., 2003; Huang et al., 2009; Yang et al., 2012, 2013), and lack complete development. As mentioned, upregulating Atoh1 via gene insertion has been shown to create non-cochlear cell types that behave in a manner that is not found within the native cochlea. In addition, these methods increase hair cell numbers but decrease supporting cell numbers. Because supporting cells are known to have specialized roles (Ramirez-Camancho 2006, Dale and Jagger 2010), loss of these cells could create problems in proper cochlear function.

Thus, there remains a long felt need for new compounds that can effectively regenerate new hair cells in the inner ear, both in vitro and in vivo.

### SUMMARY

In some aspects, the present disclosure provides a compound of Formula (I):

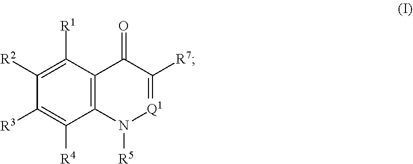

or a pharmaceutically acceptable salt or tautomer thereof, wherein:

Q¹ is CR⁶ or N;

R¹ is selected from the group consisting of H, F, Cl, Br, NO₂, OR^{1a}, SR^{1a}, N(R^{1a})₂, and C₁-C₆ alkyl; wherein each R^{1a} is independently H, C₁-C₆ alkyl, or —C(═O)—(C₁-C₆ alkyl); wherein the C₁-C₆ alkyl is optionally substituted with one or more halogen or phenyl;

R² is selected from the group consisting of H, F, Cl, Br, C₁-C₆ alkyl, and NR¹⁰R¹¹;

R³ is selected from the group consisting of -L-R⁸, C₁-C₈ alkyl, F, N(R^{3a})(R^{3b}), C₁-C₄ alkyl-N(R^{3a})(R^{3b}), O^{3b}, C₃-C₈ cycloalkyl optionally substituted with N(R^{3a})(R^{3b}), CH₂N(R^{3a})CH₂CN, N(R^{3a})CH₂CN, and aryl optionally substituted with R^{3b}; wherein R^{3a} is H or C₁-C₈ alkyl; R^{3b} is H, C₁-C₈ alkyl, C₂-C₈ alkenyl, C₃-C₈ cycloalkyl, C₄-C₈ heterocycloalkyl, C₄-C₈ cycloalkenyl, phenyl, indanyl, heteroaryl, or —C(═O)—(C₁-C₆ alkyl); wherein the C₁-C₈ alkyl, C₂-C₈ alkenyl, C₃-C₈ cycloalkyl, C₄-C₈ heterocycloalkyl, C₄-C₈ cycloalkenyl, phenyl heteroaryl, or —C(═O)—(C₁-C₆ alkyl) is optionally substituted with one or more halogen, C₁₋₄ alkyl, OR⁹, NR¹⁰R¹¹; phenyl optionally substituted with C₁-C₄ alkyl, C₃-C₆ cycloalkyl optionally substituted with NR¹⁰R¹¹, or heteroaryl optionally substituted with one or more halogen, C₁-C₄ alkyl, OR⁹, or NR¹⁰R¹¹; wherein when R^{3b} is C₃-C₈ cycloalkyl substituted with at least two C₁-C₄ alkyl substituents, the two C₁-C₄ alkyl substituents together with the carbon atom(s) to which they are attached can form a C₃-C₈ cycloalkyl or C₆-C₁₆ aryl; or wherein R^{3a} and R^{3b} are taken together with the N to which they are attached to form a 3-6 membered heterocycle optionally comprising one or more additional heteroatom selected from N, O and S; that is optionally substituted with one or more OR⁹, NR¹⁰R¹¹, halogen, or C₁-C₄ alkyl;

R⁴ is selected from the group consisting of R^{4a}, F, Cl, Br, OR^{4a}, and N(R^{4a})₂; wherein each R^{4a} is independently H, C₁-C₆ alkyl, C₃-C₆ cycloalkyl, or —C(═O)—(C₁-C₆ alkyl);

R⁵ is selected from the group consisting of H, C₁-C₈ alkyl, C₃-C₈ cycloalkyl, C₄-C₈ cycloalkenyl, C₂-C₈ alkenyl, C₂-C₈ alkynyl, and —C(═O)—(C₁-C₄ alkyl); wherein the C₁-C₈ alkyl, C₃-C₈ cycloalkyl, C₄-C₈cycloalkenyl, C₂-C₈ alkenyl, or C₂-C₈ alkynyl is optionally substituted with one or more fluoro, phenyl, or OR^{1a}; or wherein R^{4a} and R⁵ are taken together with the atoms to which they are attached to form a 5-8 membered heterocycle optionally substituted with halogen, N(R^{3a})(R^{3b}), OR^{1a}, or optionally substituted C₁-C₃ alkyl; wherein when a carbon atom of the heterocycl has two C₁-C₃ alkyl substituents attached thereto, the two C₁-C₃ alkyl substituents together with the carbon atom to which they are attached can form a 3-8 membered cycloalkyl or heterocycl comprising one or more heteroatoms selected from O, N and S;

R⁶ is selected from the group consisting of H, F, Cl, Br, and C₁-C₆ alkyl;

R⁷ is selected from the group consisting of CN, tetrazolyl, CH₂OR^{7a}, CFHOR^{7a}, CF₂OR^{7a}, CO₂R^{7a}, CON(R^{7b})₂, and C(═NH)—N(R^{7b})₂; wherein R^{7a} is H, C₁-C₆ alkyl or C₃-C₆ cycloalkyl; and each R^{7b} is independently H, OH, C₁-C₆ alkyl, or C₃-C₆ cycloalkyl;

L is selected from the group consisting of a bond, —(CH₂)₁₋₄—, —C₄-C₈cycloalkenyl-, —(CH₂)ₙN(R^{La})(CH₂)ₙ—, -cycloalkyl-N(R^{La})—, —(CH₂)ₙO—, -aryl-, -heterocycl-, and -heteroaryl-; wherein L is optionally substituted with one or more halo or C₁-C₄ alkyl; wherein Ru is H or C₁-C₈ alkyl; and each n is independently 0 to 4;

R⁸ is selected from the group consisting of C₃-C₈ cycloalkyl-N(R^{8a})(R^{8b}), aryl-C₃-C₈ cycloalkyl-N(R^{8a})(R^{8b}), N(R^{8a})—C₃-C₈ cycloalkyl-aryl, C₄-C₈ cycloalkenyl, OR^{8b}, and N(R^{8a})(R^{8b}); wherein R⁸ is optionally substituted with F or C₁-C₆ alkyl; R^{8a} is H or C₁-C₈ alkyl; and R^{8b} is H or C₁-C₈ alkyl

R⁹ is H, C₁-C₆ alkyl, or —C(═O)—(C₁-C₆ alkyl); and

R¹⁰ and R¹¹ are each independently selected from H, C₁-C₈ alkyl, C₂-C₈ alkenyl, C₃-C₈ cycloalkyl, C₄-C₈ cycloalkenyl, or —C(═O)—(C₁-C₆ alkyl), wherein the C₁-C₈ alkyl, C₂-C₈ alkenyl, C₃-C₈ cycloalkyl, or C₄-C₈cycloalkenyl is optionally substituted with one or more F, C₁-C₄ alkyl, optionally substituted phenyl, or optionally substituted heteroaryl, or indanyl.

In some aspects, the present disclosure provides a pharmaceutical composition comprising a compound of the present disclosure, or a pharmaceutically acceptable salt or tautomer thereof, and a pharmaceutically acceptable carrier.

In some aspects, the present disclosure provides a pharmaceutical composition comprising a compound of the present disclosure, or a pharmaceutically acceptable salt or tautomer thereof, at least one additional pharmaceutically active agent, or a pharmaceutically acceptable salt or tautomer thereof, and a pharmaceutically acceptable carrier.

In some aspects, the present disclosure provides a method of expanding a population of cochlear cells in a cochlear tissue comprising a parent population, the method comprising contacting the cochlear tissue with a compound of the present disclosure, or a pharmaceutically acceptable salt or tautomer thereof or a pharmaceutical composition of the present disclosure.

In some aspects, the present disclosure provides a method of facilitating the generation of tissue cells, the method comprising administering or causing to be administered to a stem cell population a compound of the present disclosure, or a pharmaceutically acceptable salt or tautomer thereof or a pharmaceutical composition of the present disclosure.

In some aspects, the present disclosure provides a method of treating or preventing a disease associated with absence or lack of certain tissue cells in a subject in need thereof, comprising administering or causing to be administered to a stem cell population a compound of of the present disclosure, or a pharmaceutically acceptable salt or tautomer thereof or a pharmaceutical composition of the present disclosure.

In some aspects, the present disclosure provides a method of treating or preventing hearing loss in a subject in need thereof, the method comprising administering a compound of the present disclosure, or a pharmaceutically acceptable salt or tautomer thereof or a pharmaceutical composition of the present disclosure.

In some aspects, the present disclosure provides a method of facilitating the generation of inner ear hair cells, the method comprising: administering a compound of the present disclosure or a pharmaceutically acceptable salt thereof, alone or in combination with an additional pharmaceutically active agent, to expand the stem cell population of cochlear tissue.

In some aspects, the present disclosure provides a method of regenerating or improving hearing in a mammal, the method comprising administering a compound of the present disclosure, or a pharmaceutically acceptable salt or tautomer thereof, alone or in combination with an additional pharmaceutically active agent.

In some aspects, the present disclosure provides a method of generating inner ear hair cells, the method comprising administering a compound of the present disclosure, or a pharmaceutically acceptable salt or tautomer thereof, alone or in combination with an additional pharmaceutically active agent, wherein the method proliferates Lgr5⁺ cells in an initial population in vivo, resulting in an expanded population of Lgr5⁺ cells, resulting in generation of inner ear hair cells.

In some aspects, the present disclosure provides a method of facilitating generation of intestinal cells, the method comprising administering a compound of the present disclosure or a pharmaceutically acceptable salt thereof, alone or in combination with an additional pharmaceutically active agent, to expand the stem cell population of intestinal epithelia.

In some aspects, the present disclosure provides a method of expanding Lgr5⁺ cell population of intestinal epithelia, the method comprising: administering a compound of the present disclosure or a pharmaceutically acceptable salt thereof, alone or in combination with an additional pharmaceutical agent.

In some aspects, the present disclosure provides a method of use of a compound of the present disclosure, or a pharmaceutically acceptable salt or tautomer thereof, alone or in combination with an additional pharmaceutically active agent to regenerate Lgr5⁺ cell population intestinal cells in a mammal.

In some aspects, the present disclosure provides a method of proliferating Lgr5⁺ epithelial cells in in vivo, the method comprising: administering a compound of the present disclosure or a pharmaceutically acceptable salt thereof.

In some aspects, the present disclosure provides a method for expanding a population of vestibular cells in a vestibular tissue comprising contacting the vestibular tissue with (i) a compound of the present disclosure or a pharmaceutically acceptable salt thereof, and (ii) an additional pharmaceutically active agent to form an expanded population of cells in the vestibular tissue.

In some aspects, the present disclosure provides a method of treating or preventing vestibular diseases, alopecia, oncology, acute myeloid leukemia, inflammation, Alzheimer's disease, Huntington's disease, Friedreick's ataxia, depression, anxiety, manic episodes of bipolar/mood disorders, Parkinson's disease, diabetes, bacterial infection, Anti-Trypanosoma brucei, ischemia, heart disease, vascular degeneration, and/or platelet aggregation in a subject in need thereof, the method comprising administering a compound of the present disclosure or a pharmaceutically acceptable salt thereof, alone or in combination with an additional pharmaceutically active agent.

In some aspects, the present disclosure provides a method of inhibiting LSD, GSK3, and/or FOXO in a cell, the method comprising contacting the cell with a compound of the present disclosure or a pharmaceutically acceptable salt thereof.

In some aspects, the present disclosure provides a system for treating or preventing a disease associated with absence or lack of certain tissue cells in a subject in need thereof, comprising administering: a compound of the present disclosure, or a pharmaceutically acceptable salt or tautomer thereof; and a transtympanic administrative device.

In some aspects, the present disclosure provides a method for proliferation of stem cells comprising administering to a cell population an effective amount of a compound of the present disclosure.

In some aspects, proliferation occurs in the absence of an additional activator or an additional inhibitor.

In some aspects, the present disclosure provides a compound of the present disclosure, or a pharmaceutically acceptable salt or tautomer thereof, for use in treating or preventing a disease associated with absence or lack of certain tissue cells in a subject in need thereof.

In some aspects, the present disclosure provides a compound of the present disclosure, or a pharmaceutically acceptable salt or tautomer thereof, for use in treating or preventing hearing loss in a subject in need thereof.

In some aspects, the present disclosure provides a use of a compound of the present disclosure, or a pharmaceutically acceptable salt or tautomer thereof, in the manufacture of a medicament for treating or preventing a disease associated with absence or lack of certain tissue cells in a subject in need thereof.

In some aspects, the present disclosure provides a use of a compound of the present disclosure, or a pharmaceutically acceptable salt or tautomer thereof, in the manufacture of a medicament for treating or preventing hearing loss in a subject in need thereof.

In some aspects, the present disclosure provides a use of a compound of the present disclosure, or a pharmaceutically acceptable salt or tautomer thereof, in the manufacture of a medicament for treating or preventing a disease responding to LSD inhibition, GSK3 inhibition, and/or FOXO inhibition, or a combination of the foregoing in a subject in need thereof.

In some aspects, the present disclosure provides a use of a compound of the present disclosure, or a pharmaceutically acceptable salt or tautomer thereof, in the manufacture of a medicament for treating or preventing vestibular diseases, alopecia, oncology, acute myeloid leukemia, inflammation, Alzheimer's disease, Huntington's disease, Friedreick's ataxia, depression, anxiety, manic episodes of bipolar/mood disorders, Parkinson's disease, diabetes, bacterial infection, Anti-Trypanosoma brucei, ischemia, heart disease, vascular degeneration, and/or platelet aggregation in a subject in need thereof.

All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of compounds disclosed herein, the chemical structures will control.

Other objects, features, and advantages will be in part apparent and in part pointed out hereinafter from the following detailed description and claims.

A compound of the present disclosure, or a pharmaceutically acceptable salt or tautomer thereof, can act as an inhibitor of FOXO-1, GSK3alpha, GSK3beta, GSK3alpha/beta, and/or LSD-1, or a combination of any of the foregoing. In some embodiments, the compounds inhibit FOXO-1 and GSK3 α/β. In some embodiments, the compounds inhibit FOXO-1 and LSD-1. In some embodiments, the compounds inhibit LSD-1 and GSK3 α/β. In some embodiments, the compounds inhibit FOXO-1, GSK3 α/β, and LSD-1. The combined effects on multiple targets provided by the compounds disclosed herein can result in improvements in methods of treatment where inhibition of more than one of FOXO-1, GSK3 α/β, and LSD-1 would be beneficial. For example, the compounds disclosed herein can be more effective as a monotherapy than compounds with reduced, or no, activity inhibiting FOXO-1, GSK3 α/β, and LSD-1. For example, the compounds disclosed herein can be useful for treating hearing loss, in particular sensorineural hearing loss.

Compounds of the present disclosure, or a pharmaceutically acceptable salt or tautomer thereof, when evaluated alone can increase the number of Lgr5+ cells in the sensory epithelium of the inner ear, e.g., in cochlear epithelium as set out in the examples. For example, the compounds disclosed increased the number of Lgr5+ cells to levels attained for two compound combinations (e.g., CHIR99021 (a GSK3 α/β inhibitor) and sodium valproate (an HDAC inhibitor)), or levels attained for three compound combinations (e.g., CHIR99021 (a GSK3 α/β inhibitor), sodium valproate (an HDAC inhibitor), and tranylcypromine (an LSD-1 inhibitor)). Therefore, a compound of the present disclosure, or a pharmaceutically acceptable salt or tautomer thereof, or compositions of the present disclosure, can be advantageous in treating hearing loss, in particular sensorineural hearing loss. It is also disclosed that a compound of the present disclosure, or a pharmaceutically acceptable salt or tautomer thereof, in combination with at least one additional pharmaceutically active agent (either independently or in a pharmaceutical composition) can also be advantageous in treating hearing loss, in particular sensorineural hearing loss.

### BRIEF DESCRIPTION OF THE DRAWINGS

FIG. 1 is a graph showing the concentration response Lgr5 positive cell percent for Compound I-7 compared to CHIR-99021 (4 μM) and the combination of CHIR-99021 (4 μM) and sodium valproate (1 mM).

FIG. 2 is a graph showing the concentration response Lgr5 positive cell count and total cell count for Compound I-7 at concentrations of 123.5 nM, 370.4 nM, 1.11 μM, 3.33 μM, and 10 μM compared to CHIR-99021 (4 μM) and the combination of CHIR-99021 (4 μM) and sodium valproate (1 mM).

FIG. 3 is a graph showing the concentration response percent of Lgr5 positive cells for Compound I-7 in combination with CHIR-99021 (4 μM) compared to CHIR-99021 (4 μM) alone, and CHIR-99021 (4 μM) in combination with sodium valproate (1 mM).

FIG. 4 is a graph showing the concentration response Lgr5 positive cell count and total cell count for Compound I-7 at concentrations of 123.5 nM, 370.4 nM, 1.11 μM, 3.33 μM, and 10 μM in combination with CHIR-99021 (4 μM) compared to CHIR-99021 (4 μM) alone, and CHIR-99021 (4 μM) in combination with sodium valproate (1 mM).

FIG. 5 is a graph showing the concentration response percent of Lgr5 positive cells for Compound I-7 in combination with sodium valproate (1 mM) compared to CHIR-99021 (4 μM) alone, and CHIR-99021 (4 μM) in combination with sodium valproate (1 mM).

FIG. 6 is a graph showing the concentration response Lgr5 positive cell count and total cell count for Compound I-7 at concentrations of 200 nM, 275 nM, 350 nM, 425 nM, 500 nM, 650 nM, 800 nM, and 1000 nM in combination with sodium valproate (1 mM) compared to CHIR-99021 (4 μM) alone, and CHIR-99021 (4 μM) in combination with sodium valproate (1 mM).

FIG. 7 is a graph showing concentration response for Lgr5 positive cells for tranylcypromine in combination with CHIR-99021 (4 μM) and sodium valproate (1 mM) compared to the combination of CHIR-99021 (4 μM) and sodium valproate (1 mM).

FIG. 8 is a graph showing the concentration response Lgr5 positive cell count and total cell count for tranylcypromine at concentrations of 0.1 μM, 0.5 μM, 1 μM, 2 μM, 3 μM, 4 μM, 5 μM, 6 μM, 7 μM, 8 μM, 10 μM, 12 μM, 16 μM, and 20 μM in combination with sodium valproate (1 mM) compared to CHIR-99021 (4 μM) alone, and CHIR-99021 (4 μM) in combination with sodium valproate (1 mM).

FIG. 9 is a graph showing concentration response for GFP positive cells and total cell area for Compound I-20 at concentrations of 14 nM, 41 nM, 123 nM, 370 nM, 1.11 μM, 3.33 μM, and 10 μM compared to CHIR-99021 (4 μM) alone, CHIR-99021 (4 μM) in combination with sodium valproate (1 mM), and the combination of CHIR-99021 (4 μM), sodium valproate (1 mM), and tranylcypromine (7 μM).

FIG. 10 is a graph showing concentration response for GFP positive cells and total cell area for Compound I-20 at concentrations of 0.5 μM, 0.75 μM, 1 μM, 1.5 μM, 2 μM, and 3 μM compared to CHIR-99021 (4 μM) alone, CHIR-99021 (4 μM) in combination with sodium valproate (1 mM), and the combination of CHIR-99021 (4 μM), sodium valproate (1 mM), and tranylcypromine (7 μM).

FIG. 11 is a graph showing concentration response for GFP positive cells and total cell area for Compound I-20 at concentrations of 0.5 μM, 0.75 μM, 1 μM, 1.5 μM, 2 μM, and 3 μM in combination with sodium valproate (1 mM) compared to CHIR-99021 (4 μM) alone, CHIR-99021 (4 μM) in combination with sodium valproate (1 mM), and the combination of CHIR-99021 (4 μM), sodium valproate (1 mM), and tranylcypromine (7 μM).

FIG. 12 is a graph showing concentration response for GFP positive cells and total cell area for Compound I-20 at concentrations of 0.5 μM, 0.75 μM, 1 μM, 1.5 μM, 2 μM, and 3 μM in combination with sodium valproate (1 mM) and tranylcypromine (7 μM) compared to CHIR-99021 (4 μM) alone, CHIR-99021 (4 μM) in combination with sodium valproate (1 mM), and the combination of CHIR-99021 (4 μM), sodium valproate (1 mM), and tranylcypromine (7 μM).

FIG. 13A is a graph showing the concentration response Lgr5 positive cell count and total cell count for Compound I-28 (8 μM) in combination with EGF (“E”, 50 ng/mL), bFGF (“F”, 50 ng/mL), and IGR1 (“I”, 50 ng/mL) compared to CHIR-99021 (“C”, 4 μM) in combination with EGF (“E”, 50 ng/mL), bFGF (“F”, 50 ng/mL), and IGR1 (“I”, 50 ng/mL); CHIR-99021 (“C”, 4 μM) in combination with sodium valproate (“V”, 1 mM), EGF (“E”, 50 ng/mL), bFGF (“F”, 50 ng/mL), and IGR1 (“I”, 50 ng/mL); and CHIR-99021 (“C”, 4 μM) in combination with tranylcypromine (“I”, 7 μM), sodium valproate (“V”, 1 mM), EGF (“E”, 50 ng/mL), bFGF (“F”, 50 ng/mL), and IGR1 (“I”, 50 ng/mL).

FIG. 13B is a graph showing the concentration response percent of Lgr5 positive cells for Compound I-28 (8 μM) in combination with EGF (“E”, 50 ng/mL), bFGF (“F”, 50 ng/mL), and IGR1 (“I”, 50 ng/mL) compared to CHIR-99021 (“C”, 4 μM) in combination with EGF (“E”, 50 ng/mL), bFGF (“F”, 50 ng/mL), and IGR1 (“I”, 50 ng/mL); CHIR-99021 (“C”, 4 μM) in combination with sodium valproate (“V”, 1 mM), EGF (“E”, 50 ng/mL), bFGF (“F”, 50 ng/mL), and IGR1 (“I”, 50 ng/mL); and CHIR-99021 (“C”, 4 μM) in combination with tranylcypromine (“T”, 7 μM), sodium valproate (“V”, 1 mM), EGF (“E”, 50 ng/mL), bFGF (“F”, 50 ng/mL), and IGR1 (“I”, 50 ng/mL).

FIG. 14A is a graph showing the concentration response Lgr5 positive cell count and total cell count for Compound I-29 (8 μM) in combination with EGF (“E”, 50 ng/mL), bFGF (“F”, 50 ng/mL), and IGR1 (“I”, 50 ng/mL) compared to CHIR-99021 (“C”, 4 μM) in combination with EGF (“E”, 50 ng/mL), bFGF (“F”, 50 ng/mL), and IGR1 (“I”, 50 ng/mL); CHIR-99021 (“C”, 4 μM) in combination with sodium valproate (“V”, 1 mM), EGF (“E”, 50 ng/mL), bFGF (“F”, 50 ng/mL), and IGR1 (“I”, 50 ng/mL); and CHIR-99021 (“C”, 4 μM) in combination with tranylcypromine (“T”, 7 μM), sodium valproate (“V”, 1 mM), EGF (“E”, 50 ng/mL), bFGF (“F”, 50 ng/mL), and IGR1 (“I”, 50 ng/mL).

FIG. 14B is a graph showing the concentration response percent of Lgr5 positive cells for Compound I-29 (8 μM) in combination with EGF (“E”, 50 ng/mL), bFGF (“F”, 50 ng/mL), and IGR1 (“I”, 50 ng/mL) compared to CHIR-99021 (“C”, 4 μM) in combination with EGF (“E”, 50 ng/mL), bFGF (“F”, 50 ng/mL), and IGR1 (“I”, 50 ng/mL); CHIR-99021 (“C”, 4 μM) in combination with sodium valproate (“V”, 1 mM), EGF (“E”, 50 ng/mL), bFGF (“F”, 50 ng/mL), and IGR1 (“I”, 50 ng/mL); and CHIR-99021 (“C”, 4 μM) in combination with tranylcypromine (“T”, 7 μM), sodium valproate (“V”, 1 mM), EGF (“E”, 50 ng/mL), bFGF (“F”, 50 ng/mL), and IGR1 (“I”, 50 ng/mL).

### DETAILED DESCRIPTION

The present disclosure relates to quinolin-4-one and 4(1H)-cinnolinone compounds and methods of using them to induce self-renewal of stem/progenitor supporting cells, including inducing the stem/progenitor cells to proliferate while maintaining, in the daughter cells, the capacity to differentiate into tissue cells.

Without wishing to be bound by any theory, it is understood that some compounds of the present disclosure may work via one or more mechanisms to expand a population of stem progenitor supporting cells (e.g., cochlear cells in cochlear tissue) including, GSK3α/β inhibition, Foxo-1 inhibition, and/or LSD-1 inhibition.

Some compounds of the present disclosure demonstrate enhanced stem/progenitor supporting cells expansion, such as increasing the number of cells or increasing the percent of Lgr5+ cells in cochlear tissue, better than a Foxo-1 inhibitor, LSD-1 inhibitor, or GSK3α/β inhibitor with high GSK3 selectivity. Surprisingly, Applicants have been able to invent compounds that are more effective than a single inhibitor and can affect more than one target such as at least two of Foxo-1, LSD-1, GSK3α, GSK3β, and GSK3α/β.

Some compounds of the present disclosure demonstrate enhanced stem/progenitor supporting cell expansion, as demonstrated by increasing the number of cells or increasing the percent of Lgr5+ cells in cochlear tissue, similar to or better than combinations of compounds (e.g., a GSK3α/β inhibitor with sodium valproate, a GSK3α/β inhibitor with an LSD-1 inhibitor, or a GSK3α/β inhibitor with sodium valproate and an LSD-1 inhibitor); or as a single agent instead of using multiple agents.

Some compounds of the present disclosure may demonstrate an improved method of treating or preventing a disease associated with the absence or lack of function of certain tissue cells (e.g. hearing loss) in human subjects, reduce the number of agents, or potentially replace combinations of compounds with a single agent.

A description of various aspects and embodiments of the present disclosure follows.

### Definitions

In this application, the use of “or” includes “and/or” unless stated otherwise. As used in this application, the term “comprise” and variations of the term, such as “comprising” and “comprises,” are not intended to exclude other additives, components, integers or steps. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether they materially affect the activity or action of the listed elements.

The terms “about” and “approximately” are used as equivalents. Any numerals used in this disclosure with or without about/approximately are meant to cover any normal fluctuations appreciated by one of ordinary skill in the relevant art. In some embodiments, the term “approximately” or “about” refers to a range of values that fall within 25°%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Any reference to a compound is also a reference to a pharmaceutically acceptable salt of that compound (regardless of whether or not pharmaceutically acceptable salts are explicitly mentioned). Any compound can be provided for use in the invention in any pharmaceutically acceptable solid form, e.g., salt, solvate, hydrate, polymorph, amorphous material form, etc. Any references to a compound also include references to artificially deuterated forms of that compound.

“Activity” refers to biological function mediated by proteins of a cell measured by methods known in the art such as immunostaining and western blotting in conjunction with cellular effects such as proliferation, cellular growth, or cellular gene expression.

“Administration” refers to introducing a substance into a subject. In some embodiments, administration is auricular, intraauricular, intracochlear, intravestibular, or transtympanic, e.g., by injection. In some embodiments, administration is directly to the inner ear, e.g., injection through the round window or oval window, otic capsule, or vestibular canals. In some embodiments, administration is directly into the inner ear via a cochlear implant delivery system. In some embodiments, the substance is injected transtympanically to the middle ear. In some embodiments “causing to be administered” refers to administration of a second component after a first component has already been administered (e.g., at a different time and/or by a different actor).

An “antibody” refers to an immunoglobulin polypeptide, or fragment thereof, having immunogen binding ability.

As used herein, an “agonist” is an agent that causes an increase in the expression or activity of a target gene, protein, or a pathway, respectively. Therefore, an agonist can bind to and activate its cognate receptor in some fashion, which directly or indirectly brings about this physiological effect on the target gene or protein. An agonist can also increase the activity of a pathway through modulating the activity of pathway components, for example, through inhibiting the activity of negative regulators of a pathway. Therefore, a “Wnt agonist” can be defined as an agent that increases the activity of Wnt pathway, which can be measured by increased TCF/LEF-mediated transcription in a cell. Therefore, a “Wnt agonist” can be a true Wnt agonist that bind and activate a Frizzled receptor family member, including any and all of the Wnt family proteins, an inhibitor of intracellular beta-catenin degradation, and activators of TCF/LEF.

An “antagonist” refers to an agent that binds to a receptor, protein, or protein complex, and which in turn decreases or eliminates binding by other molecules or blocks its function.

“Antisense” refers to a nucleic acid sequence, regardless of length, that is complementary to the coding strand or mRNA of a nucleic acid sequence. Antisense RNA can be introduced to an individual cell, tissue or organanoid. An antisense nucleic acid can contain a modified backbone, for example, phosphorothioate, phosphorodithioate, or other modified backbones known in the art, or may contain non-natural internucleoside linkages.

As referred to herein, a “complementary nucleic acid sequence” is a nucleic acid sequence capable of hybridizing with another nucleic acid sequence comprised of complementary nucleotide base pairs. By “hybridize” is meant pair to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R. (1987) Methods Enzymol. 152:507).

“Auricular administration” refers to a method of using a catheter or wick device to administer a composition across the tympanic membrane to the inner ear of the subject. To facilitate insertion of the wick or catheter, the tympanic membrane may be pierced using a suitably sized syringe or pipette. The device could also be inserted using any other suitable methods known to those of skill in the art, e.g., surgical implantation of the device. In particular embodiments, the wick or catheter device may be a stand-alone device, meaning that it is inserted into the ear of the subject and then the composition is controllably released to the inner ear. In other particular embodiments, the wick or catheter device may be attached or coupled to a pump or other device that allows for the administration of additional compositions. The pump may be automatically programmed to deliver dosage units or may be controlled by the subject or medical professional.

“Biocompatible Matrix” as used herein is a polymeric carrier that is acceptable for administration to humans for the release of therapeutic agents. A Biocompatible Matrix can be a biocompatible gel or foam.

“Cell Aggregate” as used herein shall mean a body cells in the oof Corti that have proliferated to form a cluster of a given cell type that is greater than 40 microns in diameter and/or produced a morphology in which greater than 3 cell layers reside perpendicular to the basilar membrane. A “Cell Aggregate” can also refer a process in which cell division creates a body of cells that cause one or more cell types to breach the reticular lamina, or the boundary between endolymph and perilymph

“Cell Density” as used herein in connection with a specific cell type is the mean number of that cell type per area in a Representative Microscopy Sample. The cell types may include but are not limited to Lgr5⁺ cells, hair cells, or supporting cells. The Cell Density may be assessed with a given cell type in a given organ or tissue, including but not limited to the cochlea or organ of Corti. For instance, the Lgr5⁺ Cell Density in the organ of Corti is the Cell Density of Lgr5⁺ cells as measured across the organ of Corti. Typically, supporting cells and Lgr5⁺ cells will be enumerated by taking cross sections of the organ of Corti. Typically, hair cells will be enumerated by looking down at the surface of the organ of Corti, though cross sections may be used in some instances, as described in a Representative Microscopy Sample. Typically, Cell Density of Lgr5⁺ cells will be measured by analyzing whole mount preparations of the organ of Corti and counting the number of Lgr5 cells across a given distance along the surface of the epithelia, as described in a Representative Microscopy Sample. Hair cells may be identified by their morphological features such as bundles or hair cell specific stains (e.g., Myosin VIIa, Prestin, vGlut3, Pou4f3, Espin, conjugated-Phalloidin, PMCA2, Ribeye, Atoh1, etc.). Lgr5⁺ cells may be identified by specific stains or antibodies (e.g., Lgr5-GFP transgenic reporter, anti-Lgr5 antibody, etc.)

“Cis Absolute” as used herein refers to a chiral compound with the absolute configuration of the two highest priority groups, using the Cahn-Ingold-Prelog system, on a ring are cis to each other wherein one chiral form has been separated from the other. For example, Cyclopropyl Cis Absolute refers to a compound with a cis configuration of the two highest priority groups, using the Cahn-Ingold-Prelog system, on the cyclopropyl ring with one chiral forms. For example, wherein (1S,2S)-2-phenylcyclopropan-1-amine has been separated from (1R,2R)-2-phenylcyclopropan-1-amine.

“Cis Relative” as used herein refers to a compound with the relative position of the two highest priority groups, using the Cahn-Ingold-Prelog system, on a ring are cis in relation to each other wherein the two chiral forms are not resolved. For example, Cyclopropyl Cis Relative refers to a mixture of compounds with a cis configuration of the two highest priority groups, using the Cahn Ingold-Prelog system, on the cyclopropyl ring which encompass two chiral forms both with the cis configuration. For example, (1S,2S)-rel-2-phenyl-cyclopropan-1-amine is an unresolved mixture of (1S,2S)-2-phenylcyclopropan-1-amine and (1R,2R)-2-phenylcyclopropan-1-amine.

“Cochlear Concentration” as used herein will be the concentration of a given agent as measured through sampling cochlear fluid. Unless otherwise noted, the sample should contain a substantial enough portion of the cochlear fluid so that it is approximately representative of the average concentration of the agent in the cochlea. For example, samples may be drawn from a vestibular canal, and a series of fluid samples drawn in series such that individual samples are comprised of cochlear fluid in specified portions of the cochlea

“Complementary nucleic acid sequence” refers to a nucleic acid sequence capable of hybridizing with another nucleic acid sequence comprised of complementary nucleotide base pairs.

“Cross-Sectional Cell Density” as used herein in connection with a specific cell type is the mean number of that cell type per area of cross section through a tissue in a Representative Microscopy Sample. Cross sections of the organ of Corti can also be used to determine the number of cells in a given plane. Typically, hair cells Cross-sectional Cell Density will be measured by analyzing whole mount preparations of the organ of Corti and counting the number of hair cells across a given distance in cross sections taken along a portion of the epithelia, as described in a Representative Microscopy Sample. Typically, Cross-sectional Cell Density of Lgr5⁺ cells will be measured by analyzing whole mount preparations of the organ of Corti and counting the number of Lgr5⁺ cells across a given distance in cross sections taken along a portion of the epithelia, as described in a Representative Microscopy Sample. Hair cells may be identified by their morphological features such as bundles or hair cell specific stains (suitable stains include e.g., Myosin Vila, Prestin, vGlut3, Pou4f3, conjugated-Phalloidin, PMCA2, Atoh1, etc.). Lgr5⁺ cells may be identified by specific stains or antibodies (suitable stains and antibodies include fluorescence in situ hybridization of Lgr5 mRNA, Lgr5-GFP transgenic reporter system, anti-Lgr5 antibodies, etc.).

“Decreasing” or “decreases” refers to decreasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the level of reference or control.

“Decreasing” or “decreases” also includes decreasing by at least about 1.1-fold, for example, at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more, for example, as compared to the level of a reference or control.

“Differentiation Period” as used herein is the duration of time in which there is an Effective Stemness Driver Concentration without an Effective Differentiation Inhibition Concentration.

“Effective Concentration” may be the Effective Sternness Driver Concentration for a Sternness Driver or the Effective Diffusion Inhibition Concentration for a Diffusion Inhibitor.

“Effective Differentiation Inhibition Concentration” is the minimum concentration of a Differentiation Inhibitor that does not allow more than a 50% increase in the fraction of the total population of cells that are hair cells at the end of the Stem Cell Proliferation Assay compared to the start of the Stem Cell Proliferation Assay. In measuring the Effective Differentiation Inhibition Concentration, a Hair Cell stain for cells may be used with flow cytometry to quantify hair cells for a mouse strain that is not an Atoh1-GFP mouse. Alternatively, and Atoh1-GFP mouse strain may be used.

“Effective Release Rate” (mass/time) as used herein is the Effective Concentration (mass/volume)*30 uL/1 hour.

“Effective Stemness Driver Concentration” is the minimum concentration of a Stemness Driver that induces at least 1.5-fold increase in number of LGR5+ cells in a Stem Cell Proliferation Assay compared to the number of Lgr5+ cells in a Stem Cell Proliferation Assay performed without the Sternness Driver and with all other components present at the same concentrations.

“Eliminate” means to decrease to a level that is undetectable.

“Engraft” or “engraftment” refers to the process of stem or progenitor cell incorporation into a tissue of interest in vivo through contact with existing cells of the tissue. “Epithelial progenitor cell” refers to a multipotent cell which has the potential to become restricted to cell lineages resulting in epithelial cells.

“Epithelial stem cell” refers to a multipotent cell which has the potential to become committed to multiple cell lineages, including cell lineages resulting in epithelial cells.

“Foxo1 inhibitor” refers to a compound that inhibits the Foxo1 enzyme inhibits transactivation, or inhibits its function.

“Fragment” refers to a portion of a polypeptide or nucleic acid molecule. In some embodiments, this portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“GSK3 inhibitor” is a composition that inhibits the activity of GSK3, GSK3alpha, and/or GSK3beta.

“GSK3alpha”, “GSK3α”, and “GSK3A” as used interchangeably herein are acronyms for glycogen synthase kinase 3 alpha.

“GSK3beta”, “GSK3β”, and “GSK3B” as used interchangeably herein are acronyms for glycogen synthase kinase 3 beta.

“GSK3beta inhibitor” is a composition that inhibits the activity of GSK3beta.

“Hybridize” refers to pairing to form a double-stranded molecule between complementary nucleotide bases (e.g., adenine (A) forms a base pair with thymine (T), as does guanine (G) with cytosine (C) in DNA) under suitable conditions of stringency. (See, e.g., Wahl, G. M. and S. L. Berger (1987) Methods Enzymol. 152:399; Kimmel, A. R. (1987) Methods Enzymol. 152:507).

An “inhibitor” refers to an agent that causes a decrease in the expression levels, and/or activity of a target gene, protein, and/or pathway. An “antagonist” is one example of, but is more specifically an agent that binds to a receptor, and which in turn decreases or eliminates binding by other molecules.

As used herein, an “inhibitory nucleic acid” is a double-stranded RNA, RNA interference, miRNA, siRNA, shRNA, or antisense RNA, or a portion thereof, or a mimetic thereof, that when administered to a mammalian cell results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. In some instances, expression of a target gene is reduced by 10%, 25%, 50%, 75%, or even 90-100%.

“In Vitro Lgr5 activity” refers to the level of expression or activity of Lgr5 in an in vitro population of cells. It may be measured, for example, in cells derived from a Lgr5-GFP expressing mouse such as a B6.129P2-Lgr5tm1(cre/ERT2)Cle/J mouse (also known as Lgr5-EGFP-IRES-creERT2 or Lgr5-GFP mouse, Jackson Lab Stock No: 008875) by dissociating cells to single cells, staining with propidium iodide (PI), and analyzing the cells using a flow cytometer for Lgr5-GFP expression. Inner ear epithelial cells from wild-type (non-Lgr5-GFP) mice that passing the same culturing and analyzing procedures can be used as a negative control. Typically, two population of cells are shown in the bivariate plot with GFP/FITC as one variable, which include both GFP positive and GFP negative populations. Lgr5-positive cells are identified by gating GFP positive cell population. The percentage of Lgr5-positive cells are measured by gating GFP positive cell population against both GFP negative population and the negative control. The number of Lgr5-positive cells is calculated by multiplying the total number of cells by the percentage of Lgr5-positive cells. For cells derived from non-Lgr5-GFP mice, Lgr5 activity can be measured using an anti-Lgr5 antibody or quantitative-PCR on the Lgr5 gene.

“In Vivo Lgr5 activity” as used herein is the level of expression or activity of Lgr5 in a subject. It may be measured, for example, by removing an animal's inner ear and measuring Lgr5 protein or Lgr5 mRNA. Lgr5 protein production can be measured using an anti-Lgr5 antibody to measure fluorescence intensity as determined by imaging cochlear samples, where fluorescence intensity is used as a measure of Lgr5 presence. Western blots can be used with an anti-Lgr5 antibody, where cells can be harvested from the treated organ to determine increases in Lgr5 protein. Quantitative-PCR or RNA in situ hybridization can be used to measure relative changes in Lgr5 mRNA production, where cells can be harvested from the inner ear to determine changes in Lgr5 mRNA. In some embodiments, Lgr5 expression is measured using an Lgr5 promoter driven GFP reporter transgenic system, where the presence or intensity GFP fluoresce can be directly detected using flow cytometry, imaging, or indirectly using an anti-GFP antibody.

“Increasing” or “increases” also means increases by at least about 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a as compared to the level of a reference standard.

“Increasing” or “increases” also means increases by at least about 5%, for example, 5, 6, 7, 8, 9, 10, 15, 20, 25, 30, 35, 40, 45, 50, 55, 60, 65, 70, 75, 80, 85, 90, 95, 99, 100%, or more, for example, as compared to the level of a reference.

“Intraauricular administration” refers to administration of a composition to the middle or inner ear of a subject by directly injecting the composition.

“Intracochlear” administration refers to direct injection of a composition across the tympanic membrane and across the round window membrane into the cochlea.

“Intravestibular” administration refers to direct injection of a composition across the tympanic membrane and across the round window or oval window membrane into the vestibular organs.

“Isolated” refers to a material that is free to varying degrees from components which normally accompany it as found in its native state. “Isolate” denotes a degree of separation from original source or surroundings.

“Lgr5” is an acronym for the leucine-rich repeat-containing G-protein coupled receptor 5, also known as G-protein coupled receptor 49 (GPR49) or G-protein coupled receptor 67 (GPR67). It is a protein that in humans is encoded by the Lgr5 gene.

“Lgr5 activity” is defined as the level of activity of Lgr5 in a population of cells. In an in vitro cell population, Lgr5 activity may be measured in an in vitro Lgr5 Activity assay. In an in vivo cell population, Lgr5 activity may be measured in an in vivo Lgr5 activity assay.

“Lgr5⁺ cell” or “Lgr5-positive cell” as used herein is a cell that expresses Lgr5. “Lgr5′ cell” as used herein is a cell that is not Lgr5.

“Lineage Tracing” as used herein is using a mouse line that enables fate tracing of any cell that expresses a target gene at the time of reporter induction. This can include hair cell or supporting cell genes (Sox2, Lgr5, MyosinVIIa, Pou4f3, etc.). For example, lineage tracing may use an Lgr5-EGFP-IRES-creERT2 mouse crossed with a reporter mouse, which upon induction, allows one to trace the fate of cells that expressed Lgr5 at the time of induction. By further example, Lgr5 cells can be isolated into single cells and cultured in a Stem Cell Proliferation Assay to generate colonies, then subsequently differentiated in a Differentiation Assay and analyzed for cell fate by staining for hair cell and/or supporting cell proteins and determining the reporter colocalization with either hair cell or supporting cell staining to determine the Lgr5 cells' fate. In addition, lineage tracing can be performed in cochlear explants to track supporting cell or hair cell fate within the intact organ after treatment. For example, Lgr5 cell fate can be determined by isolating the cochlea from a Lgr5-EGFP-IRES-creERT2 mouse crossed with a reporter mouse, and inducing the reporter in Lgr5 cells before or during treatment. The organ can then be analyzed for cell fate by staining for hair cell and/or supporting cell proteins and determining the reporter colocalization with either hair cell or supporting cell staining to determine the Lgr5 cells' fate. In addition, lineage tracing can be performed in vivo track supporting cell or hair cell fate within the intact organ after treatment. For example, Lgr5 cell fate can be determined inducing a reporter in an Lgr5-EGFP-IRES-creERT2 mouse crossed with a reporter mouse, treating the animal, then isolating the cochlea. The organ can then be analyzed for cell fate by staining for hair cell and/or supporting cell proteins and determining the reporter colocalization with either hair cell or supporting cell staining to determine the Lgr5 cells' fate. Lineage tracing may be performed using alternative reporters of interest as is standard in the art.

“LSD-1 inhibitor” refers to compounds that inhibit flavin-dependent amine oxidase domain-containing enzyme Lysine-specific demethylase 1 (LSD-1), also known as KDM1A.

“Mammal” refers to any mammal including but not limited to human, mouse, rat, sheep, monkey, goat, rabbit, hamster, horse, cow, or pig.

“Mean Release Time” as used herein is the time in which one-half of an agent is released into phosphate buffered saline from a carrier in a Release Assay.

“Native Morphology” as used herein is means that tissue organization largely reflects the organization in a healthy tissue.

“Non-human mammal”, as used herein, refers to any mammal that is not a human.

As used in relevant context herein, the term “number” of cells can be 0, 1, or more cells.

“Organ of Corti” as used herein refers to the sensory epithelia of the cochlea where the sensory cells (inner and outer hair cells) and supporting cells reside.

“Organoid” or “epithelial organoid” refers to a cell cluster or aggregate that resembles an organ, or part of an organ, and possesses cell types relevant to that particular organ.

“Population” of cells refers to any number of cells greater than 1. In some embodiments, “population” of cells refers to at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

“Progenitor cell” as used herein refers to a cell that, like a stem cell, has the tendency to differentiate into a specific type of cell, but is already more specific than a stem cell and is pushed to differentiate into its “target” cell.

“Proliferation Period” as used herein is the duration of time in which tissue or cells are exposed a compound according to the invention.

In some embodiments, the “purity” of any given agent or compound in a composition may be specifically defined. For instance, certain compositions may comprise an agent that is at least 80, 85, 90, 91, 92, 93, 94, 95, 96, 97, 98, 99, or 100% pure, including all decimals in between, as measured, for example and by no means limiting, by high performance liquid chromatography (HPLC), a well-known form of column chromatography used frequently in biochemistry and analytical chemistry to separate, identify, and quantify compounds.

“Reference” means a standard or control condition (e.g., untreated with a test agent or combination of test agents).

“Release Assay” as used herein is a test in which the rate of release of an agent from a Biocompatible Matrix through dialysis membrane to a saline environment. An exemplary Release Assay may be performed by placing 30 microliters of a composition in 1 ml phosphate buffered saline inside saline dialysis bag with a suitable cutoff, and placing the dialysis bag within 10 mL of phosphate buffered saline at 37° C. The dialysis membrane size may be chosen based on agent size in order to allow the agent being assessed to exit the membrane. For small molecule release, a 3.5-5 kDa cutoff may be used. The Release Rate for a composition may change over time and may be measured in 1 hour increments.

“Representative Microscopy Sample” as used herein describes a sufficient number of fields of view within a cell culture system, a portion of extracted tissue, or an entire extracted organ that the average feature size or number being measured can reasonably be said to represent the average feature size or number if all relevant fields were measured. For example, in order to assess the hair cell counts at a frequency range on the organ of Corti, Image) software (NIH) can used to measure the total length of cochlear whole mounts and the length of individual counted segments. The total number of inner hair cells, outer hair cells, and supporting cells can be counted in the entire or fraction of any of the four cochlear segments of 1200-1400 μm (apical, mid-apical, mid-basal, and basal) at least three fields of view at 100 μm field size would be reasonably considered a Representative Microscopy Sample. A Representative Microscopy sample can include measurements within a field of view, which can be measured as cells per a given distance. A Representative Microscopy sample can be used to assess morphology, such as cell-cell contacts, cochlear architecture, and cellular components (e.g., bundles, synapses).

“Rosette Patterning” is a characteristic cell arrangement in the cochlea in which <5% hair cells are adjacent to other hair cells.

The term “sample” refers to a volume or mass obtained, provided, and/or subjected to analysis. In some embodiments, a sample is or comprises a tissue sample, cell sample, a fluid sample, and the like. In some embodiments, a sample is taken from (or is) a subject (e.g., a human or animal subject). In some embodiments, a tissue sample is or comprises brain, hair (including roots), buccal swabs, blood, saliva, semen, muscle, or from any internal organs, or cancer, precancerous, or tumor cells associated with any one of these. A fluid may be, but is not limited to, urine, blood, ascites, pleural fluid, spinal fluid, and the like. A body tissue can include, but is not limited to, brain, skin, muscle, endometrial, uterine, and cervical tissue or cancer, precancerous, or tumor cells associated with any one of these. In an embodiment, a body tissue is brain tissue or a brain tumor or cancer. Those of ordinary skill in the art will appreciate that, in some embodiments, a “sample” is a “primary sample” in that it is obtained from a source (e.g., a subject); in some embodiments, a “sample” is the result of processing of a primary sample, for example to remove certain potentially contaminating components and/or to isolate or purify certain components of interest.

“Self-renewal” refers to the process by which a stem cell divides to generate one (asymmetric division) or two (symmetric division) daughter cells with development potentials that are indistinguishable from those of the mother cell. Self-renewal involves both proliferation and the maintenance of an undifferentiated state.

“siRNA” refers to a double-stranded RNA. Optimally, an siRNA is 18, 19, 20, 21, 22, 23 or 24 nucleotides in length and has a 2-base overhang at its 3′ end. These dsRNAs can be introduced to an individual cell or culture system. Such siRNAs are used to downregulate mRNA levels or promoter activity.

“Stem cell” refers to a multipotent cell having the capacity to self-renew and to differentiate into multiple cell lineages.

“Stem Cell Differentiation Assay” as used herein is an assay to determine the differentiation capacity of stem cells. In an exemplary Stem Cell Differentiation Assay, the number of cells for an initial cell population is harvested from a Atoh1-GFP mouse between the age of 3 to 7 days, by isolating the organ of Corti sensory epithelium, dissociating the epithelium into single cells, and passing the cells through a 40 um cell strainer. Approximately 5000 cells are entrapped in 40 μl of culture substrate (for example: Matrigel (Corning, Growth Factor Reduced)) and placed at the center of wells in a 24-well plate with 500 μl of an appropriate culture media, growth factors and agent being tested. Appropriate culture media and growth factors include Advanced DMEM/F12 with media supplements (1×N2, 1×B27, 2 mM Glutamax™, 10 mM HEPES, 1 mM N-acetylcysteine, and 100 U/ml penicillin/100 μg/ml streptomycin) and growth factors (50 ng/ml EGF, 50 ng/ml bFGF, and 50 ng/ml IGF-1) as well as the agent(s) being assessed are added into each well. Cells are cultured for 10 days in a standard cell culture incubator at 37° C. and 5% CO₂, with media change every 2 days. These cells are then cultured by removing the Stem Cell Proliferation Assay agents and replacing with basal culture media and molecules to drive differentiation. An appropriate basal culture media is Advanced DMEM/F12 supplemented with 1×N2, 1×B27, 2 mM Glutamax, 10 mM HEPES, 1 mM N-acetylcysteine, and 100 U/ml penicillin/100 μg/ml streptomycin and appropriate molecules to drive differentiation are 3 μM CHIR99021 and 5 μM DAPT for 10 days, with media change every 2 days. The number of hair cells in a population may be measured by using flow cytometry for GFP. Hair cell differentiation level can further be assessed using qPCR to measure hair cell marker (e.g., Myo7a) expression level normalized using suitable and unregulated references or housekeeping genes (e.g., Hprt). Hair cell differentiation level can also be assessed by immunostaining for hair cell markers (e.g., Myosin7a, vGlut3, Espin, PMCAs, Ribeye, conjugated-phalloidin, Atoh1, Pou4f3, etc.). Hair cell differentiation level can also be assessed by western blot for Myosin7a, vGlut3, Espin, PMCAs, Prestin, Ribeye, Atoh1, Pou4f3.

“Stem Cell Assay” as used herein is an assay in which a cell or a cell population are tested for a series of criteria to determine whether the cell or cell population are stem cells or enriched in stem cells or stem cell markers. In a stem cell assay, the cell/cell population are tested for stem cell characteristics such as expression of Stem Cell Markers, and further optionally are tested for stem cell function, including the capacity of self-renewal and differentiation.

“Stem Cell Proliferator” as used herein is a compound that induces an increase in a population of cells which have the capacity for self-renewal and differentiation.

“Stem Cell Proliferation Assay” as used herein is an assay to determine the capacity for agent(s) to induce the creation of stem cells from a starting cell population. In an exemplary Stem Cell Proliferation Assay, the number of cells for an initial cell population is harvested from a Lgr5-GFP mouse such as a B6.129P2-Lgr5tm1(cre/ERT2)Cle/J mouse (also known as Lgr5-EGFP-IRES-creERT2 or Lgr5-GFP mouse, Jackson Lab Stock No: 008875) between the age of 3 to 7 days, by isolating the organ of Corti sensory epithelium and dissociating the epithelium into single cells. Approximately 5000 cells are entrapped in 40 μl of culture substrate (for example, Matrigel (Corning, Growth Factor Reduced)) and placed at the center of wells in a 24-well plate with 500 μl of an appropriate culture media, growth factors and agent being tested. Appropriate culture media and growth factors include Advanced DMEM/F12 with media supplements (1×N2, 1×B27, 2 mM Glutamax, 10 mM HEPES, 1 mM N-acetylcysteine, and 100 U/ml penicillin/100 μg/ml streptomycin) and growth factors (50 ng/ml EGF, 50 ng/ml bFGF, and 50 ng/ml IGF-1) as well as the agent(s) being assessed are added into each well. Cells are cultured for 10 days in a standard cell culture incubator at 37° C. and 5% CO₂, with media change every 2 days. The number of Lgr5⁺ cells is quantified by counting the number of cells identified as Lgr5+ in an in vitro Lgr5 activity assay (i.e., an assay mearing in vitro Lgr5 activity). The fraction of cells that are Lgr5⁺ is quantified by dividing the number of cells identified as Lgr5⁺ in a cell population by the total number of cells present in the cell population. The average Lgr5⁺ activity of a population is quantified by measuring the average mRNA expression level of Lgr5 of the population normalized using suitable and unregulated references or housekeeping genes (e.g., Hprt). The number of hair cells in a population may be measured by staining with hair cell marker (e.g., MyosinVIIa), or using an endogenous reporter of hair cell genes (e.g., Pou4f3-GFP, Atoh1-nGFP) and analyzing using flow cytometry. The fraction of cells that are hair cells is quantified by dividing the number of cells identified as hair cells in a cell population by the total number of cells present in the cell population. Lgr5 activity can be measured by qPCR.

“Stem cell markers” as used herein can be defined as gene products (e.g., protein, RNA, etc.) that specifically expressed in stem cells. One type of stem cell marker is gene products that are directly and specifically support the maintenance of stem cell identity. Examples include Lgr5 and Sox2. Additional stem cell markers can be identified using assays that were described in the literatures. To determine whether a gene is required for maintenance of stem cell identity, gain-of-function and loss-of-function studies can be used. In gain-of-function studies, over expression of specific gene product (the stem cell marker) would help maintain the stem cell identity. While in loss-of-function studies, removal of the stem cell marker would cause loss of the stem cell identity or induced the differentiation of stem cells. Another type of stem cell marker is gene that only expressed in stem cells but does not necessary to have specific function to maintain the identity of stem cells. This type of marker can be identified by comparing the gene expression signature of sorted stem cells and non-stem cells by assays such as micro-array and qPCR. This type of stem cell marker can be found in the literature (e.g., Liu Q. et al., Int. J. Biochem. Cell Biol. 2015 March; 60:99-111). Potential stem cell markers include Ccdc121, Gdf10, Opcm1, Phex, etc. The expression of stem cell markers such as Lgr5 or Sox2 in a given cell or cell population can be measure using assays such as qPCR, immunohistochemistry, western blot, and RNA hybridization. The expression of stem cell markers can also be measured using transgenic cells express reporters which can indicate the expression of the given stem cell markers, e.g., Lgr5-GFP or Sox2-GFP. Flow cytometry analysis can then be used to measure the activity of reporter expression. Fluorescence microscopy can also be used to directly visualize the expression of reporters. The expression of stem cell markers may further be determined using microarray analysis for global gene expression profile analysis. The gene expression profile of a given cell population or purified cell population can be compared with the gene expression profile of the stem cell to determine similarity between the two cell populations. Stem cell function can be measured by colony forming assay or sphere forming assay, self-renewal assay and differentiation assay. In colony (or sphere) forming assay, when cultured in appropriate culture media, the stem cell should be able to form colonies, on cell culture surface (e.g., cell culture dish) or embedded in cell culture substrate (e.g., Matrigel) or be able to form spheres when cultured in suspension. In colony/sphere forming assay, single stem cells are seeded at low cell density in appropriate culture media and allowed to proliferate for a given period of time (7-10 days). Colony formed are then counted and scored for stem cell marker expression as an indicator of sternness of the original cell. Optionally, the colonies that formed are then picked and passaged to test its self-renewal and differentiation potential. In self-renewal assay, when cultured in appropriate culture media, the cells should maintain stem cell marker (e.g., Lgr5) expression over at least one (e.g., 1, 2, 3, 4, 5, 10, 20, etc.) cell divisions. In a Stem Cell Differentiation Assay, when cultured in appropriate differentiation media, the cells should be able to generate hair cell which can be identified by hair cell marker expression measured by qPCR, immunostaining, western blot, RNA hybridization or flow cytometry.

“Sternness Driver” as used herein is a composition that induces proliferation of LGR5⁺ cells, upregulates Lgr5 in cells, or maintains Lgr5 expression in cells, while maintaining the potential for self-renewal and the potential to differentiate into hair cells. Generally, stemness drivers upregulate at least one biomarker of post-natal stem cells. Sternness Drivers include but are not limited to Wnt agonists and GSK3 inhibitors.

“Subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like. In some embodiments, the subject is a subject of a clinical trial.

“Supporting Cell” as used herein in connection with a cochlear epithelium comprises epithelial cells within the organ of Corti that are not hair cells. This includes inner pillar cells, outer pillar cells, inner phalangeal cells, Deiter cells, Hensen cells, Boettcher cells, and/or Claudius cells.

“Synergy” or “synergistic effect” is an effect which is greater than the sum of each of the effects taken separately; a greater than additive effect.

“Synergist” refers to a compound that causes a more than additive increase in target gene expression or protein levels by 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold more than the additive value of each compound used individually.

By “statistically significant”, it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95% or greater of some given quantity.

“TGF Beta inhibitor” as used herein is a composition that reduces activity of TGFBeta.

“Tissue” is an ensemble of similar cells from the same origin that together carry out a specific function including, for example, tissue of cochlear, such as the organ of Corti.

“Trans Absolute” as used herein refers to a chiral compound when the absolute configuration of the two highest priority groups, using the Cahn-Ingold-Prelog system, on a ring are trans to each other wherein one chiral form has been separated from the other. For example, Cyclopropyl Trans refers to a compound with a trans configuration of the two highest priority groups, using the Cahn-Ingold-Prelog system, on the cyclopropyl ring with one chiral forms.

For example, wherein (1R,2S)-2-phenylcyclopropan-1-amine has been separated from (1S,2R)-2-phenylcyclopropan-1-amine.

“Trans Relative” as used herein refers to a compound with the relative position of the two highest priority groups, using the Cahn-Ingold-Prelog system, on a ring are trans in relation to each other wherein the two chiral forms are not resolved. For example, Cyclopropyl Trans Relative refers to a compound with a trans configuration of the two highest priority groups, using the Cahn-Ingold-Prelog system, on the cyclopropyl ring which would encompass two chiral forms both with the trans configuration. For example, (1R,2S)-rel-2-phenyl-cyclopropan-1-amine is an unresolved mixture of (1R,2S)-2-phenylcyclopropan-1-amine and (1 S,2R)-2-phenylcyclopropan-1-amine.

“Transtympanic” administration refers to direct injection of a composition across the tympanic membrane into the middle ear. “Intratympanic” administration also refers to direct injection of a composition across the tympanic membrane into the middle ear.

“Treating” as used herein in connection with a cell population means delivering a substance to the population to effect an outcome. In the case of in vitro populations, the substance may be directly (or even indirectly) delivered to the population. In the case of in vivo populations, the substance may be delivered by administration to the host subject.

It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

The phrase “facilitating the generation of tissue cells” as used herein refers to increasing the rate of the generation of the tissue cells. In some embodiments, the rate is increased as compared to a comparable subject not being administered with the compound, pharmaceutical composition, or combination described herein. In some embodiments, the rate is increased by about 2 fold, about 3 fold, about 4 fold, about 5 fold, 6 fold, about 7 fold, about 8 fold, about 9 fold, about 10 fold, about 20 fold, about 30 fold, about 40 fold, or about 50 fold.

The phrase “regenerating hearing” as used herein refers to regenerating the hearing ability of a subject, e.g., when the subject has lost hearing ability. Regenerating hearing includes regeneration to levels that are higher than a baseline but are not at fully normal parameters.

The phrase “improving hearing” as used herein refers to enhancing the hearing ability of a subject, e.g., when the subject has a reduced hearing ability. In some embodiments, the hearing ability of the subject is enhanced by about 2 fold, about 3 fold, about 4 fold, about 5 fold, 6 fold, about 7 fold, about 8 fold, about 9 fold, about 10 fold, about 20 fold, about 30 fold, about 40 fold, or about 50 fold. In some embodiments, improved hearing is an improvement in word recognition and/or pure tone threshold levels and/or improvements under noisy conditions or other measures used in the art.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Wnt activation” as used herein is an activation of the Wnt signaling pathway.

The term “alkyl” as used herein refers to a straight or branched saturated hydrocarbon. For example, an alkyl group can have 1 to 8 carbon atoms (i.e., (C₁-C₈)alkyl) or 1 to 6 carbon atoms (i.e., (C₁-C₆ alkyl) or 1 to 4 carbon atoms.

The term “alkenyl” as used herein refers to a linear or branched hydrocarbon radical which includes one or more double bonds and can include divalent radicals, having from 2 to about 15 carbon atoms. Examples of alkenyl groups include but are not limited to, ethenyl, propenyl, butenyl, and higher homologs and isomers.

The term “alkynyl” as used herein refers to a linear or branched hydrocarbon radical which includes one or more triple bonds and can include divalent radicals, having from 2 to about 15 carbon atoms. Examples of alkynyl groups include but are not limited to, ethynyl, propynyl, butynyl, and higher homologs and isomers.

The term “halo” or “halogen” as used herein refers to fluoro, chloro, bromo and iodo.

The term “aryl” as used herein refers to a single all carbon aromatic ring or a multiple condensed all carbon ring system wherein at least one of the rings is aromatic. For example, an aryl group can have 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., carbocycle). Such multiple condensed ring systems may be optionally substituted with one or more (e.g., 1, 2 or 3) oxo groups on any carbocycle portion of the multiple condensed ring system. The rings of the multiple condensed ring system can be connected to each other via fused, Spiro and bridged bonds when allowed by valency requirements. It is to be understood that the point of attachment of a multiple condensed ring system, as defined above, can be at any position of the ring system including an aromatic or a carbocycle portion of the ring.

The term “heteroaryl” as used herein refers to a single aromatic ring that has at least one atom other than carbon in the ring, wherein the atom is selected from the group consisting of oxygen, nitrogen and sulfur; the term also includes multiple condensed ring systems that have at least one such aromatic ring, which multiple condensed ring systems are further described below. Thus, the term includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. The term also includes multiple condensed ring systems (e.g., ring systems comprising 2, 3 or 4 rings) wherein a heteroaryl group, as defined above, can be condensed with one or more rings selected from heteroaryls (to form for example a naphthyridinyl such as 1,8-naphthyridinyl), heterocycles, (to form for example a 1, 2, 3, 4-tetrahydronaphthyridinyl such as 1, 2, 3, 4-tetrahydro-1,8-naphthyridinyl), carbocycles (to form for example 5,6,7, 8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system and at any suitable atom of the multiple condensed ring system including a carbon atom and heteroatom (e.g., a nitrogen).

The term “cycloalkyl” as used herein refers to a saturated or partially saturated ring structure having about 3 to about 8 ring members that has only carbon atoms as ring atoms and can include divalent radicals. Examples of cycloalkyl groups include but are not limited to, cyclopropyl, cyclobutyl, cyclopentyl, cyclohexyl, cyclohexene, cyclopentenyl, cyclohexenyl.

The terms “heterocyclyl” or “heterocyclic” refer to monocyclic or polycyclic 3 to 24-membered rings containing carbon and heteroatoms selected from oxygen, phosphorous, nitrogen, or sulfur and wherein there are no delocalized π electrons (aromaticity) shared among the ring carbon or heteroatoms. Examples of heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. A heterocyclyl or heterocycloalkyl ring can also be fused or bridged, e.g., can be a bicyclic ring. Examples of heterocyclyl also include, but are not limited to, fused rings, bridged rings (e.g., 2,5-diazabicyclo[2,2,1]heptane), and spirocyclic rings, (e.g., 2,8-diazaspiro[4,5]decane).

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In some embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In some embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic. In some embodiments, the cycloalkyl is hexahydroindacenyl. In some embodiments, the cycloalkyl is

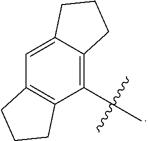 [SMILES: *c1c2c(cc3c1CCC3)CCC2]

As used herein, the term “heterocycloalkyl” refers to a saturated or partially unsaturated 3-8 membered monocyclic, 7-12 membered bicyclic (fused, bridged, or Spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen, and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or

As used herein, the term “halo” or “halogen” refers to fluoro, chloro, bromo and —O⁻.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulphhydryl, alkylthio, arythio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M, Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In some embodiments, the mammal is a human. A subject in need thereof can be one who has been previously diagnosed or identified as having a disease or disorder disclosed herein. A subject in need thereof can also be one who has (e.g., is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that doesn't respond or hasn't yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers, or propellants that are required

The use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

As used herein, the term “pharmaceutically acceptable excipient” means an excipient that is useful in preparing a pharmaceutical composition that is generally safe, non toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, /toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. For example, inorganic salts include, but are not limited to, ammolnium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammolnia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Example organic bases used in some embodiments include isopropylamine, diethylamine, ethanolamine, trimethylamine, dicyclohexylamine, choline and caffeine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), intratympanic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulphite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Intratympanic administration (and/or formulation for intratympanic administration) are particularly appropriate for the compounds disclosed herein.

It is to be understood that a compound or pharmaceutical composition of the disclosure can be administered to a subject in many of the well-known methods currently used for therapeutic treatment. For example, a compound of the disclosure may be injected into the middle ear, blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. In some embodiments, the pharmaceutical compositions exhibit large therapeutic indices. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life, clearance rate, and efficacy of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), poloxamer gels, and suitable mixtures thereof. The use of thermoreversible gels is one useful option for the compounds disclosed herein. In some embodiments the poloxamer is Poloxamer 407. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, isotonic agents, for example, sugars, polyalcohols such as mannitol and sorbitol, and sodium chloride are included in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. In some embodiments, the dose should be sufficient to result in slowing the symptoms of the disease or disorder disclosed herein. In some embodiments, the dose should be sufficient to result in slowing and preferably regressing the symptoms of the disease or disorder disclosed herein. In some embodiments, the dose should be sufficient to result in slowing and regressing the symptoms of the disease or disorder disclosed herein and also causing complete regression of the disease or disorder. Dosages can range from about 0.01 mg/kg per day to about 5000 mg/kg per day. In some embodiments, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

In some embodiments, a compound of the present disclosure is provided in a dosage of, e.g., from about 0.01 mg to about 100 mg, from about 0.1 mg to about 50 mg, from about 0.01 mg to about 10 mg, or from about 0.2 mg to about 1.0 mg. In some embodiments, the dosage of the compound is administered once or more times (e.g., once or twice) to the subject. In some embodiments, the dosage of the compound is transtympanic administered to the subject (e.g., administered to the ear of the subject through a needle). In some embodiments, the dosage of the compound is orally administered to the subject. In some embodiments, the dosage of the compound is systemically administered to the subject.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

Compounds or compositions described herein can be formulated in any manner suitable for a desired delivery route, e.g., transtympanic injection, transtympanic wicks and catheters, and injectable depots. Typically, formulations include all physiologically acceptable compositions including derivatives or prodrugs, solvates, stereoisomers, racemates, or tautomers thereof with any physiologically acceptable carriers, diluents, and/or excipients.

The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, intratympanically, nasally, transdermally, pulmonary, inhalationally, buccally, sublingually, intraperitoneally, subcutaneously, intramuscularly, intravenously, rectally, intrapleurally, intrathecally and parenterally. In some embodiments, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Compounds of the Present Disclosure

In some aspects, the present disclosure provides a compound of Formula (I):

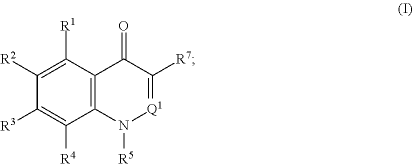

or a pharmaceutically acceptable salt or tautomer thereof, wherein:

Q¹ is CR⁶ or N;

R¹ is selected from the group consisting of H, F, Cl, Br, NO₂, OR^{1a}, SR^{1a}, N(R^{1a})₂, and C₁-C₆ alkyl; wherein each R^{1a} is independently H, C₁-C₆ alkyl, or —C(═O)—(C₁-C₆ alkyl); wherein the C₁-C₆ alkyl is optionally substituted with one or more halogen or phenyl;

R² is selected from the group consisting of H, F, Cl, Br, C₁-C₆ alkyl, and NR¹⁰R¹¹;

R³ is selected from the group consisting of C₁-C₈ alkyl, F, N(R^{3a})(R^{3b}), C₁-C₄ alkyl-N(R^{3a})(R^{3b}), OR^{3b}, C₃-C₈ cycloalkyl optionally substituted with N(R^{3a})(R^{3b}), CH₂N(R^{3a})CH₂CN, N(R^{3a})CH₂CN, and aryl optionally substituted with R^{3b}; wherein R^{3a} is H or C₁-C₈ alkyl; R^{3b} is H, C₁-C₈ alkyl, C₂-C₈ alkenyl, C₃-C₈ cycloalkyl, C₄-C₈ heterocycloalkyl, C₄-C₈ cycloalkenyl, phenyl, indanyl, or —C(═O)—(C₁-C₆ alkyl); wherein the C₁-C₈ alkyl, C₂-C₈ alkenyl, C₃-C₈ cycloalkyl, C₄-C₈ heterocycloalkyl, C₄-C₈cycloalkenyl phenyl, or —C(═O)—(C₁-C₆ alkyl) is optionally substituted with one or more halogen, C₁₋₄ alkyl, OR⁹, NR¹⁰R¹¹, phenyl optionally substituted with C₁-C₄ alkyl, C₃-C₆ cycloalkyl optionally substituted with NR¹⁰R¹¹, or heteroaryl optionally substituted with one or more halogen, C₁-C₄ alkyl, OR⁹, or NR¹⁰R¹¹; wherein when R^{3b} is C₃-C₈ cycloalkyl substituted with at least two C₁-C₄ alkyl substituents, the two C₁-C₄ alkyl substituents together with the carbon atom(s) to which they are attached can form a C₃-C₈ cycloalkyl or C₆-C₁₆ aryl; or wherein R^{3a} and R^{3b} are taken together with the N to which they are attached to form a 3-6 membered heterocycle optionally comprising one or more additional heteroatom selected from N, O and S; that is optionally substituted with one or more OR⁹, NR¹⁰R¹¹, halogen, or C₁-C₄ alkyl;

R⁴ is selected from the group consisting of R^{4a}, F, Cl, Br, OR^{4a}, and N(R^{4a})₂; wherein each R^{4a} is independently H, C₁-C₆ alkyl, C₃-C₆ cycloalkyl, or —C(═O)—(C₁-C₆ alkyl);

R⁵ is selected from the group consisting of H, C₁-C₈ alkyl, C₃-C₈ cycloalkyl, C₄-C₈ cycloalkenyl, C₂-C₈ alkenyl, C₂-C₈ alkynyl, and —C(═O)—(C₁-C₄ alkyl); wherein the C₁-C₈ alkyl, C₃-C₈ cycloalkyl, C₄-C₈cycloalkenyl, C₂-C₈ alkenyl, or C₂-C₈ alkynyl is optionally substituted with one or more fluoro, phenyl, OR^{1a}; or wherein R^{4a} and R⁵ are taken together with the atoms to which they are attached to form a 5-8 membered heterocycle optionally substituted with halogen, N(R^{3a})(R^{3b}), OR^{1a}, or optionally substituted C₁-C₃ alkyl; wherein when a carbon atom of the heterocycle has two C₁-C₃ alkyl substituents attached thereto, the two C₁-C₃ alkyl substituents together with the carbon atom to which they are attached can form a 3-8 membered cycloalkyl or heterocycle comprising one or more heteroatoms selected from O, N and S;

R⁶ is selected from the group consisting of H, F, Cl, Br, and C₁-C₆ alkyl;

R⁷ is selected from the group consisting of CN, tetrazolyl, CH₂OR^{7a}, CFHOR^{7a}, CF₂OR^{7a}, CO₂R^{7a}, CON(R^{7b})₂, and C(═NH)—N(R^{7b})₂; wherein R^{7a} is H, C₁-C₆ alkyl or C₃-C₆ cycloalkyl; and each R^{7b} is independently H, OH, C₁-C₆ alkyl, or C₃-C₆ cycloalkyl;

L is selected from the group consisting of a bond, —(CH₂)₁₋₄—, —C₄-C₈cycloalkenyl-, —(CH₂)ₙN(R^{La})(CH₂)ₙ—, -cycloalkyl-N(R^{La})—, —(CH₂)ₙO—, -aryl-, -heterocycl-, and -heteroaryl-; wherein L is optionally substituted with one or more halo or C₁-C₄ alkyl; wherein R^{La} is H or C₁-C₈ alkyl; and n is 0 to 4;

R⁸ is selected from the group consisting of C₃-C₈ cycloalkyl-N(R^{8a})(R^{8b}), aryl-C₃-C₈ cycloalkyl-N(R^{8a})(R^{8b}), N(R^{8a})—C₃-C₈ cycloalkyl-aryl, C₄-C₈ cycloalkenyl, OR^{8b}, and N(R^{8a})(R^{8b}); wherein R⁸ is optionally substituted with F or C₁-C₆ alkyl; R^{8a} is H or C₁-C₈ alkyl; and R^{8b} is H or C₁-C₈ alkyl

R⁹ is H, C₁-C₆ alkyl, or —C(═O)—(C₁-C₆ alkyl); and

R¹⁰ and R¹¹ are each independently selected from H, C₁-C₈ alkyl, C₂-C₈ alkenyl, C₃-C₈ cycloalkyl, C₄-C₈ cycloalkenyl, or —C(═O)—(C₁-C₆ alkyl), wherein the C₁-C₈ alkyl, C₂-C₈ alkenyl, C₃-C₈ cycloalkyl, or C₄-C₈cycloalkenyl is optionally substituted with one or more F, C₁-C₄ alkyl, optionally substituted phenyl, or optionally substituted heteroaryl, or indanyl.
In some embodiments, Q¹ is N.
In some embodiments, Q¹ is CR⁶.

In some embodiments, R¹ is selected from the group consisting of H, F, Cl, Br, NO₂, OR^{1a}, SR^{1a}, N(R^{1a})₂, and C₁-C₆ alkyl; wherein each R^{1a} is independently H, C₁-C₆ alkyl, or —C(═O)—(C₁-C₆ alkyl); wherein the C₁-C₆ alkyl is optionally substituted with one or more halogen or phenyl

In some embodiments, R¹ is selected from the group consisting of H, F, NO2, OH, N(R^{1a})₂, and C₁-C₃ alkyl.

In some embodiments, R¹ is selected from the group consisting of H, NH₂, NHCH₂-phenyl, NHCH₃, and CH₃.

In some embodiments, R¹ is selected from the group consisting of H, NH₂, and CH₃.

In some embodiments, R¹ is H.

In some embodiments, R¹ is CH₃.

In some embodiments, R¹ is NH₂.

In some embodiments, R¹ is NO2.

In some embodiments, R¹ is NHCH₂-phenyl.

In some embodiments, R² is selected from the group consisting of H, F, Cl, Br, C₁-C₆ alkyl, and NR¹⁰R¹¹.

In some embodiments, R² is H.

In some embodiments, R² is F.

In some embodiments, R² is NR¹⁰R¹¹.

In some embodiments, R² is NHCH₂CH₃.

In some embodiments, R³ is selected from the group consisting of N(R^{3a})(R^{3b}), C₁₋₃ alkyl-N(R^{3a})(R^{3b}), OR^{3b}, CH₂N(R^{3a})CH₂CN, N(R^{3a})CH₂CN, aryl optionally substituted with R^{3b}, and C₃-C₈ cycloalkyl optionally substituted with NR¹⁰R¹¹.

In some embodiments, R³ is N(R^{3a})(R^{3b}).

In some embodiments, R³ is NH(R^{3b}).

In some embodiments, R³ is NH(R^{3b}) and R^{3b} is selected from the group consisting of C₃-C₈ cycloalkyl, C₄-C₈ heterocycloalkyl, C₃-C₈ cycloalkyl substituted with NR¹⁰R¹¹, C₃-C₈ cycloalkyl substituted with heteroaryl, C₁-C₈ alkyl substituted with phenyl, C₁-C₈ alkyl substituted with NR¹⁰R¹¹, indanyl, and phenyl optionally substituted with C₃-C₆ cycloalkyl optionally substituted with NR¹⁰R¹¹.

In some embodiments, R³ is NH(R^{3b}) and R^{3b} is selected from the group consisting of C₃-C₈ cycloalkyl substituted with NHR¹¹, C1-C8 alkyl substituted with NHR¹¹, and phenyl optionally substituted with C₃-C₆ cycloalkyl optionally substituted with NHR¹¹, wherein R¹¹ is selected from the group consisting of H and C₃-C₈ cycloalkyl substituted with an optionally substituted phenyl.

In some embodiments, R³ is NH(R^{3b}) and R^{3b} is C₃-C₈ cycloalkyl or C₄-C₈ heterocycloalkyl.

In some embodiments, R³ is selected from the group consisting of

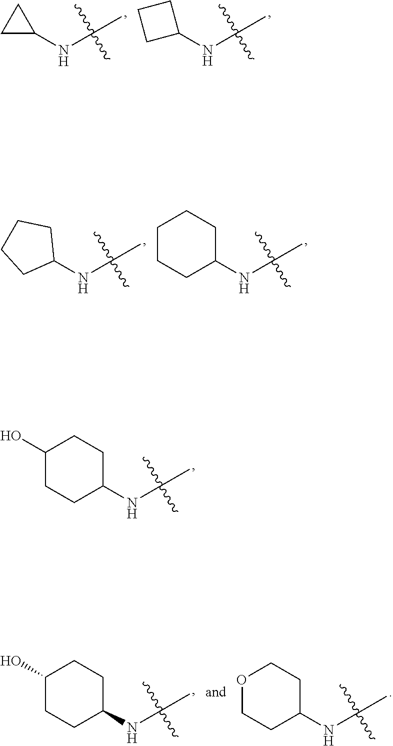

In some embodiments, R³ is selected from the group consisting of

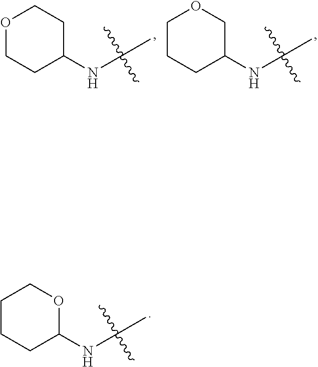

In some embodiments, R³ is N(R^{3a})(R^{3b}) and R^{3b} is C₃-C₈ cycloalkyl optionally substituted with an optionally substituted phenyl.

In some embodiments, R³ is NH(R^{3b}) and R^{3b} is cyclopropyl substituted with phenyl or cyclobutyl substituted with phenyl.

In some embodiments, R³ is selected from the group consisting of

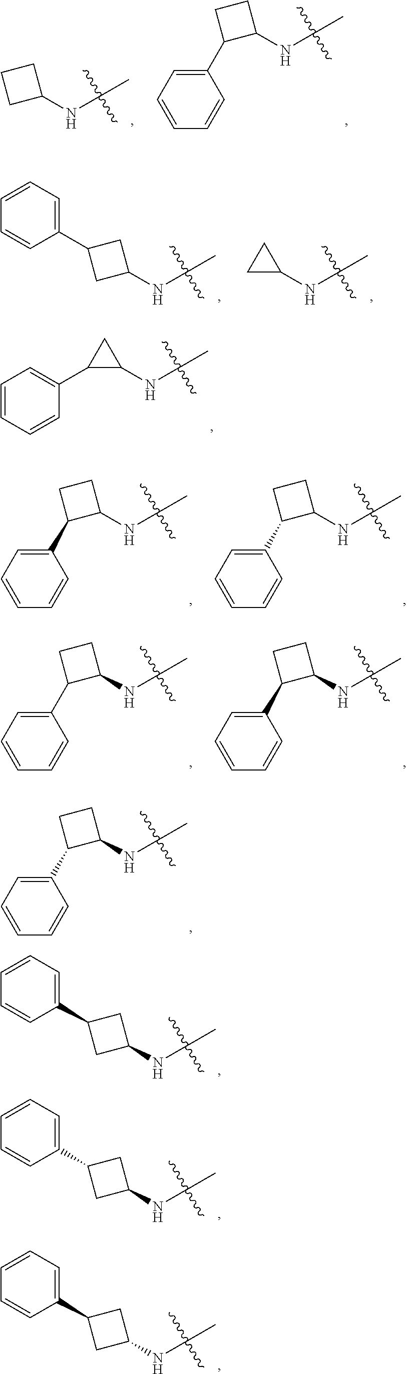

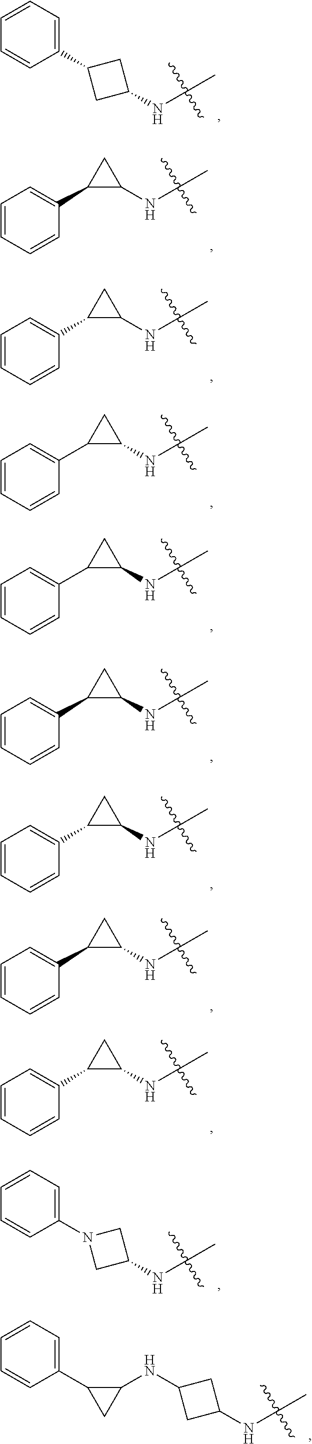

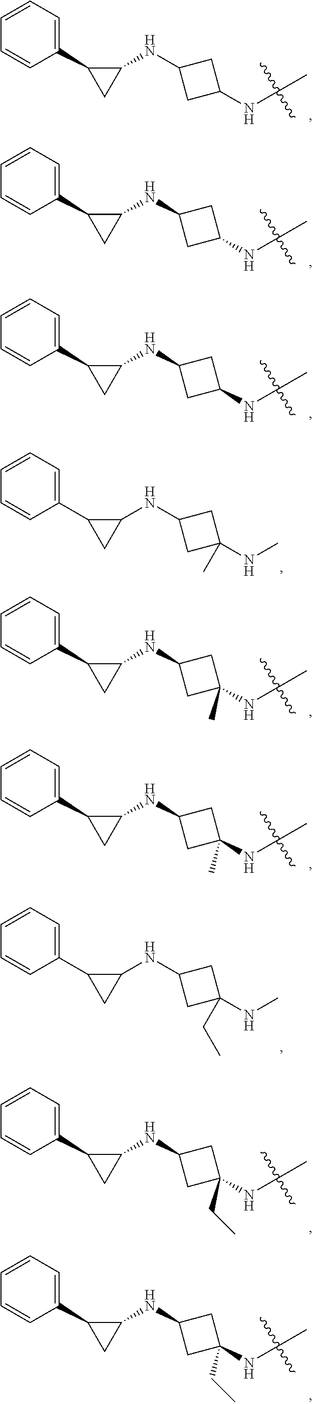

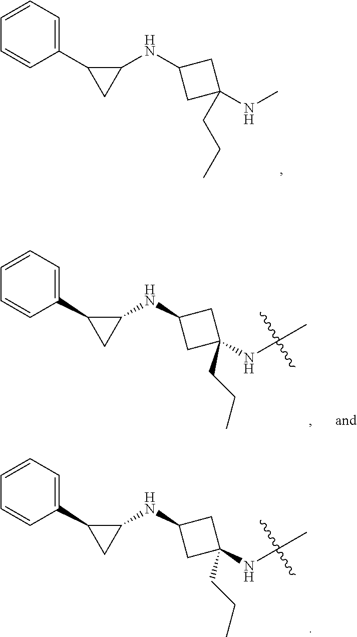 [SMILES: CCCC1(NC)CC(NC2CC2c2ccccc2)C1 ; *N[C@]1(CCC)C[C@@H](N[C@@H]2C[C@H]2c2ccccc2)C1 ; *N[C@]1(CCC)C[C@H](N[C@@H]2C[C@H]2c2ccccc2)C1]

In some embodiments, R³ is NH(R^{3a})(R^{3b}) and R^{3b} is C₃-C₈ cycloalkyl optionally substituted with one or more C₁-C₄ alkyl and phenyl optionally substituted with C₁-C₄ alkyl.

In some embodiments, R³ is NH(R^{3b}) and R^{3b} is cyclopropyl substituted with one or more methyl and one or more phenyl.

In some embodiments, R³ is NH(R^{3b}) and R^{3b} is C₃-C₈ cycloalkyl substituted with NR¹⁰R¹¹.

In some embodiments, R³ is NH(R^{3b}) and R^{3b} is C₃-C₈ cycloalkyl substituted with NHR¹¹ and R¹¹ is C₃-C₈ cycloalkyl substituted with an optionally substituted phenyl.

In some embodiments, R³ is selected from the group consisting of

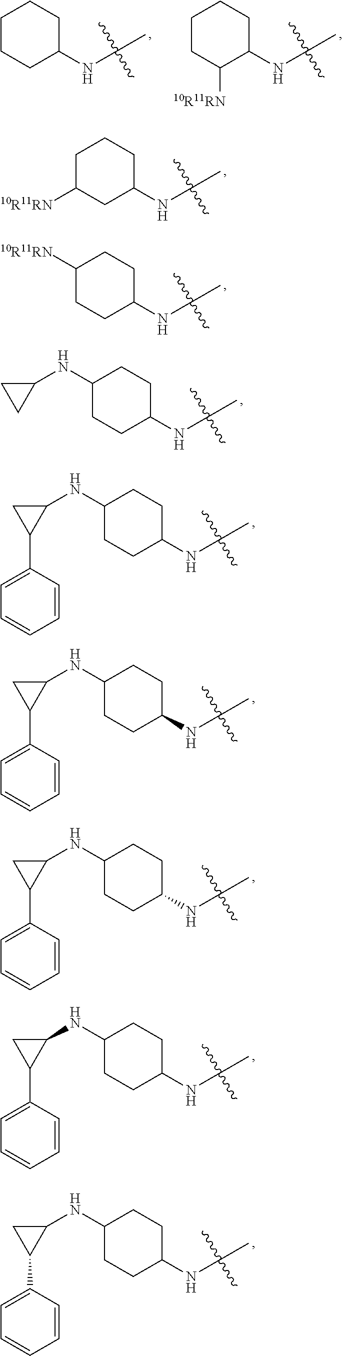

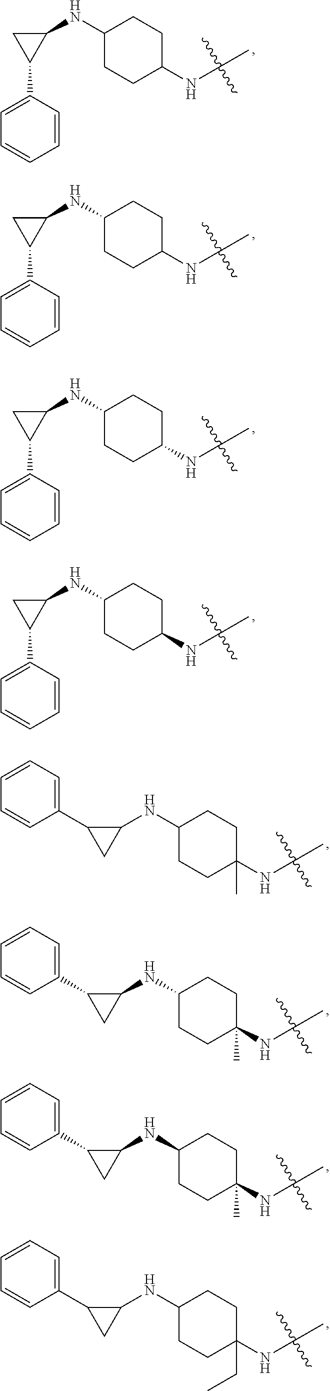

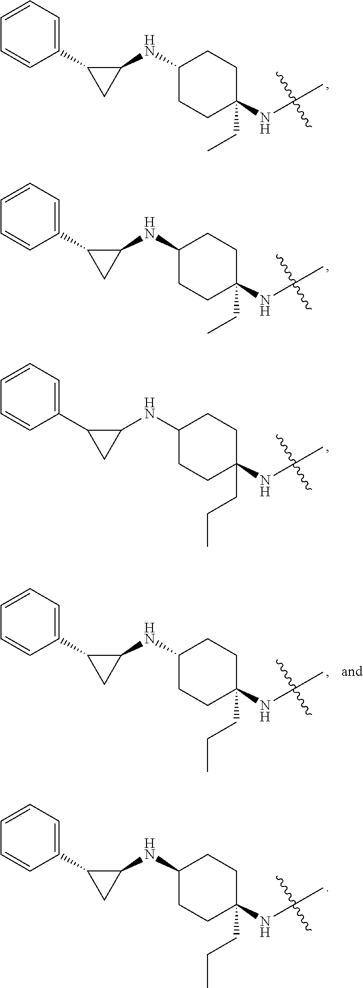

In some embodiments, R³ is selected from the group consisting of

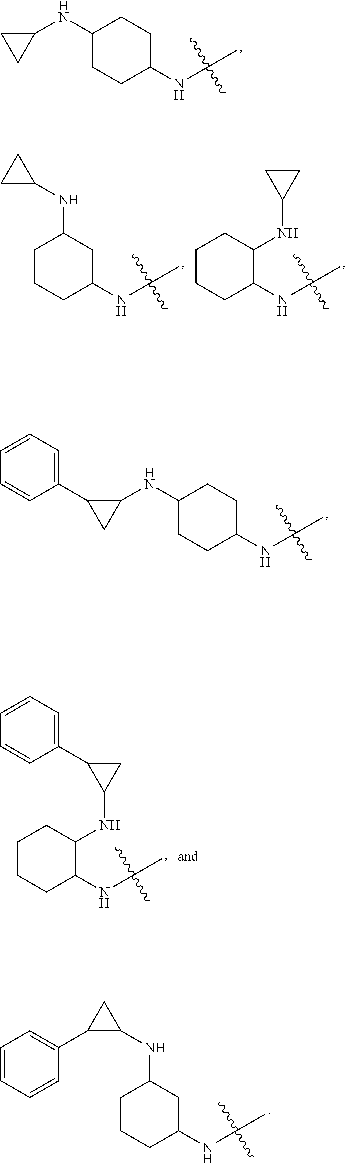

In some embodiments, R³ is NH(R^{3b}) and R^{3b} is C₃-C₈ cycloalkyl substituted with heteroaryl.

In some embodiments, R³ is selected from the group consisting of

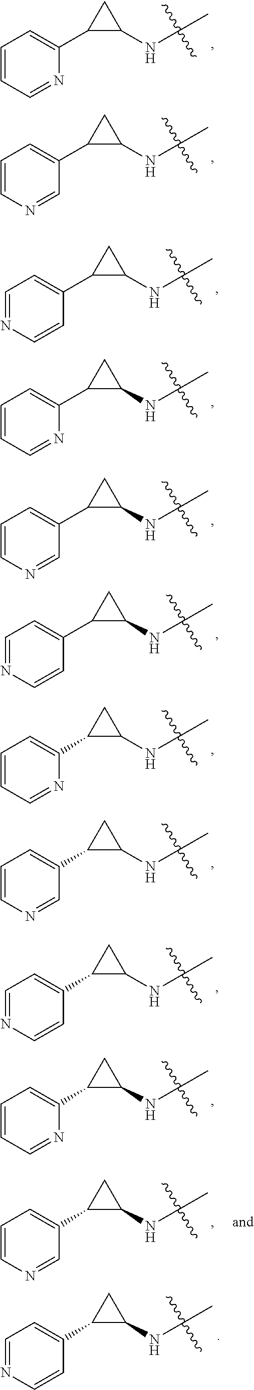

In some embodiments, R³ is NH(R^{3b}) and R^{3b} is C₁-C₈ alkyl substituted with phenyl.

In some embodiments, R³ is selected from the group consisting of

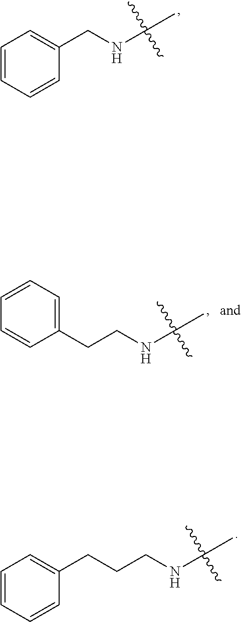 [SMILES: *NCc1ccccc1 ; *NCCc1ccccc1 ; *NCCCc1ccccc1]

In some embodiments, R³ is NH(R^{3b}) and R^{3b} is C₁-C₈ alkyl substituted with NR¹⁰R¹¹.

In some embodiments, R³ is NH(R^{3b}) and R^{3b} is C₁-C₈ alkyl substituted with NHR¹¹ and R¹¹ is C₃-C₈ cycloalkyl substituted with an optionally substituted phenyl.

In some embodiments, R³ is selected from the group consisting of

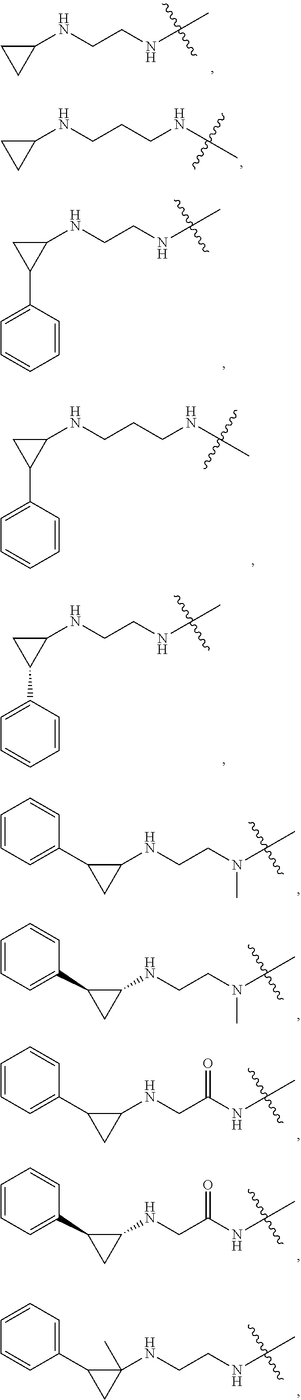

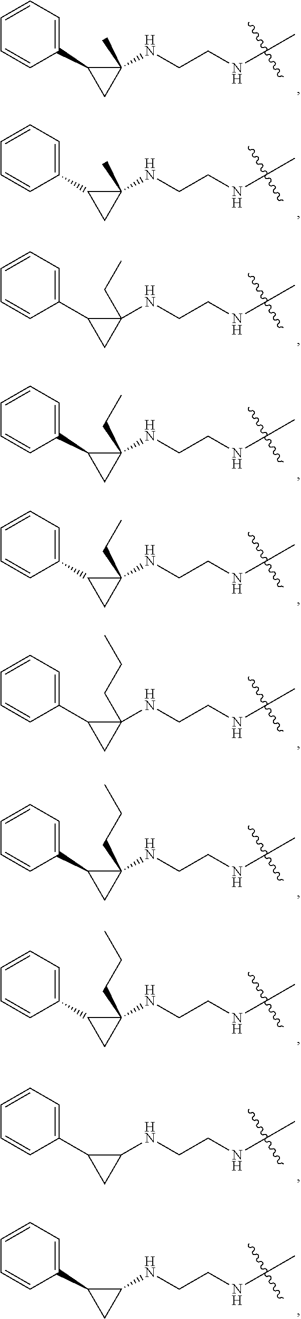

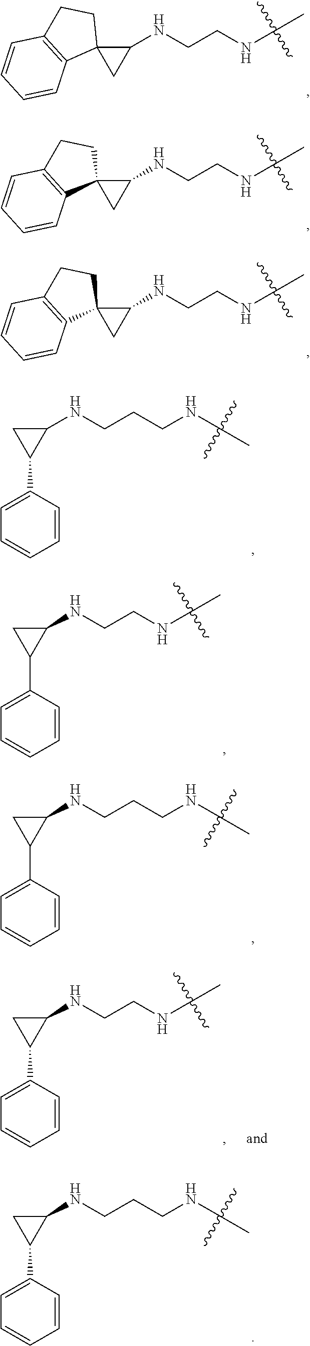

In some embodiments, R³ is NH(R^{3b}) and R^{3b} is indanyl or phenyl, wherein the phenyl is optionally substituted with C₃-C₆ cycloalkyl optionally substituted with NR¹⁰R¹¹.

In some embodiments, R³ is selected from the group consisting of

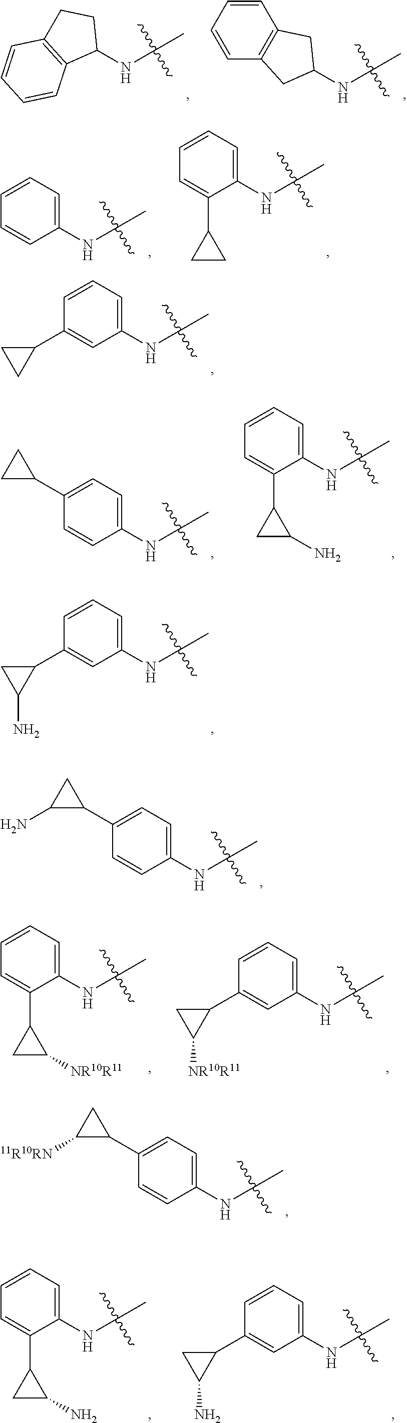

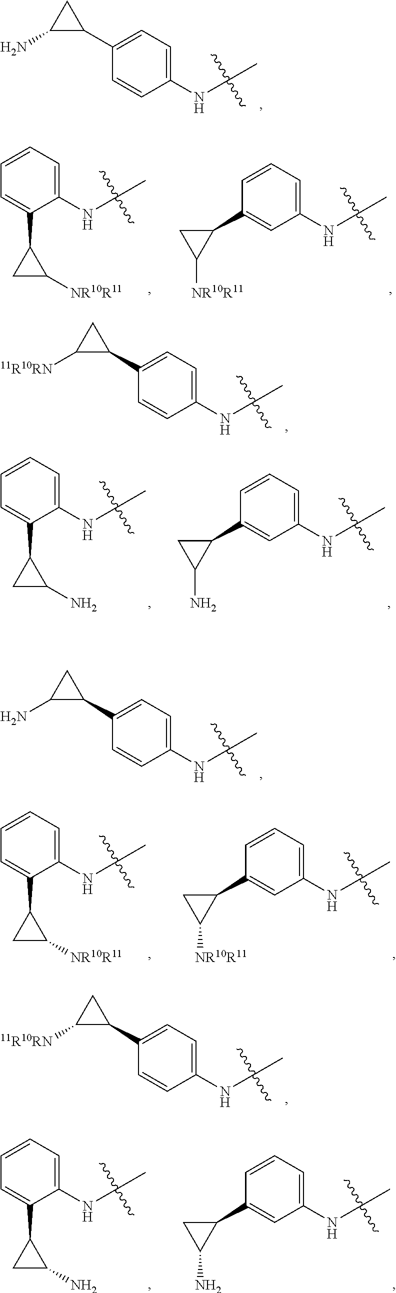

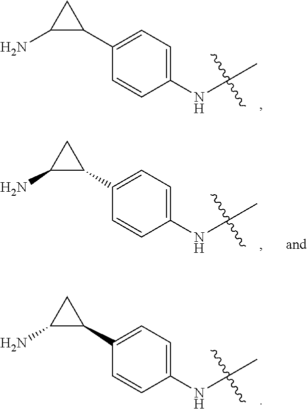 [SMILES: *Nc1ccc(C2CC2N)cc1 ; *Nc1ccc([C@H]2C[C@@H]2N)cc1 ; *Nc1ccc([C@@H]2C[C@H]2N)cc1]

In some embodiments, R³ is N(R^{3a})(R^{3b}) wherein R^{3a} and R^{3b} are taken together with the N to which they are attached to form a 3-6 membered heterocycle substituted with one or more NR¹⁰R¹¹.

In some embodiments, R³ is N(R^{3a})(R^{3b}) wherein R^{3a} and R^{3b} are taken together with the N to which they are attached to form a 6 membered heterocycle substituted with NR¹⁰R¹¹ wherein R¹⁰ is H and R¹¹ is C₃-C₈ cycloalkyl substituted with an optionally substituted phenyl.

In some embodiments, R³ is selected from the group consisting of

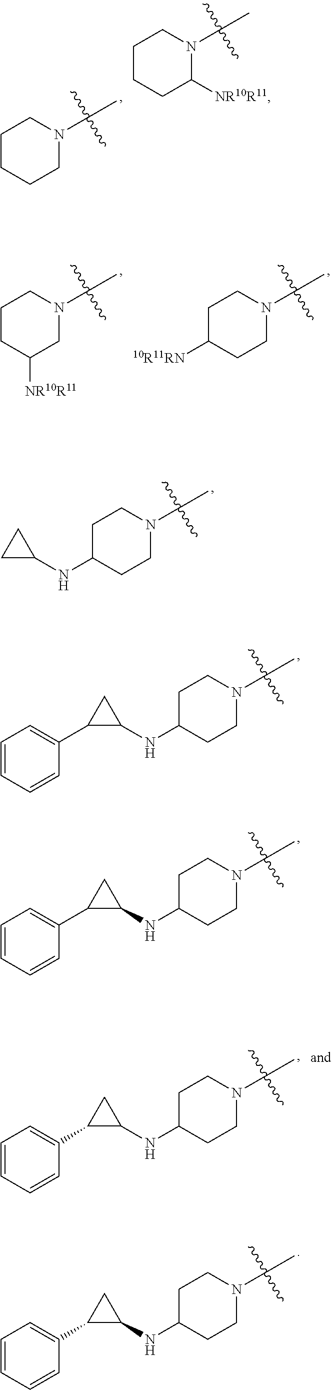

In some embodiments, R³ is selected from the group consisting of

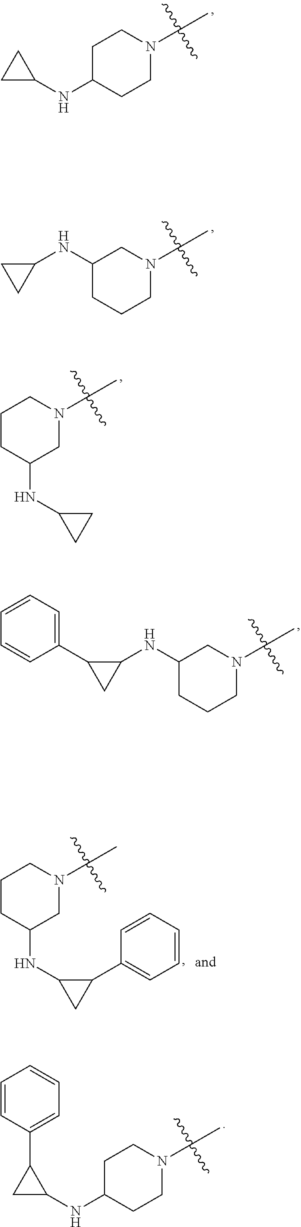 [SMILES: *N1CCC(NC2CC2)CC1 ; *N1CCCC(NC2CC2)C1 ; *N1CCCC(NC2CC2)C1 ; *N1CCCC(NC2CC2c2ccccc2)C1 ; *N1CCCC(NC2CC2c2ccccc2)C1 ; *N1CCC(NC2CC2c2ccccc2)CC1]

In some embodiments, R³ is NH(R^{3b}) and R^{3b} is C₃-C₈ cycloalkyl substituted with two C₁-C₄ alkyl substitutents and optionally further substituted with phenyl; and wherein the two C₁-C₄ alkyl substituents together with the carbon atom(s) to which they are attached form a C₃-C₈ cycloalkyl or C₆-C₁₆ aryl.

In some embodiments, R³ is elected from the group consisting of

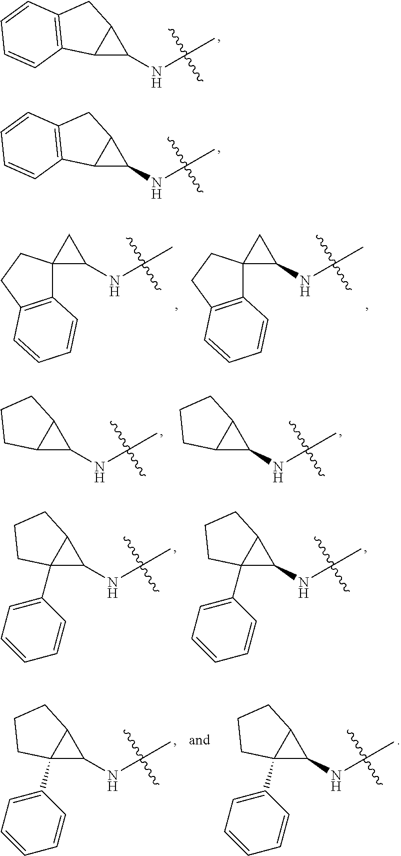

In some embodiments, R³ is C₁-C₄ alkyl-N(R^{3a})(R^{3b}).

In some embodiments, R³ is C₁-C₄ alkyl-NH(R^{3b}) and R^{3b} is C₃-C₈ cycloalkyl substituted with phenyl.

In some embodiments, R³ is selected from the group consisting of

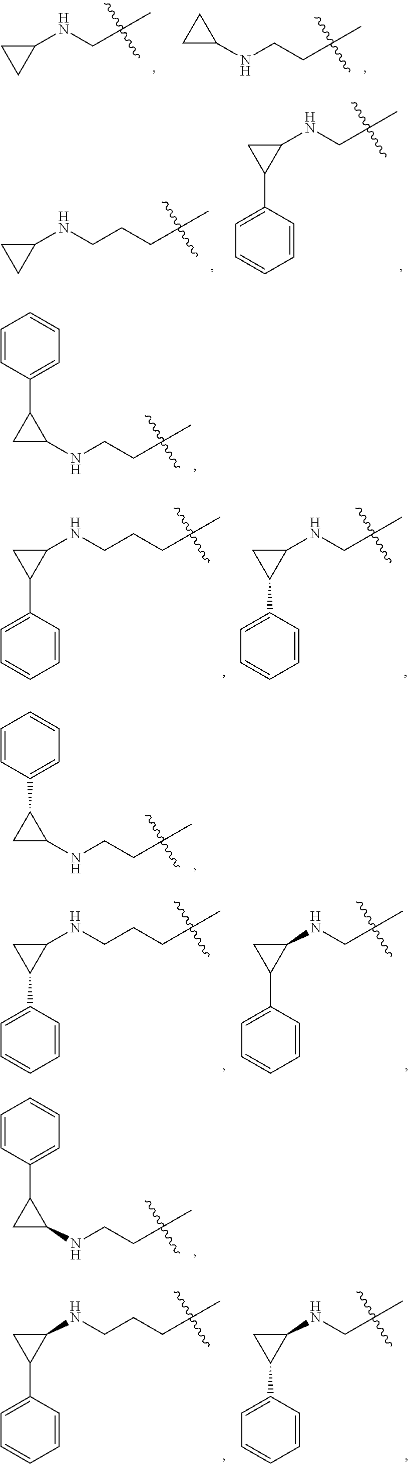

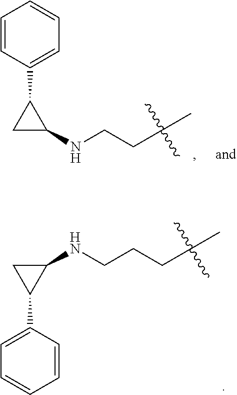 [SMILES: *CCN[C@H]1C[C@@H]1c1ccccc1 ; *CCCN[C@@H]1C[C@H]1c1ccccc1]

In some embodiments, R³ is OR^{3b}.

In some embodiments, R³ is selected from

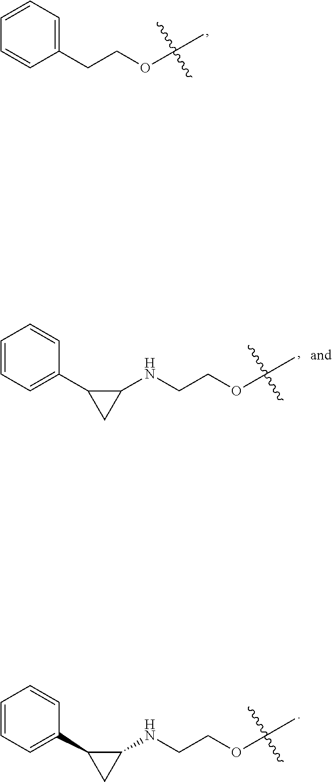 [SMILES: *OCCc1ccccc1 ; *OCCNC1CC1c1ccccc1 ; *OCCN[C@@H]1C[C@H]1c1ccccc1]

In some embodiments, R³ is selected from the group consisting of CH2N(R^{3a})CH2CN and N(R^{3a})CH₂CN.

In some embodiments, R³ is selected from the group consisting of CH₂NHCH₂CN, CH₂N(CH₃)CH₂CN, NHCH₂CN, and N(CH₃)CH₂CN.

In some embodiments, R³ is aryl optionally substituted with R^{3b}.

In some embodiments, R³ is aryl optionally substituted with R^{3b} and R^{3b} is C₃-C₆ cycloalkyl optionally substituted with NR¹⁰R¹¹.

In some embodiments, R³ is selected from the group consisting of

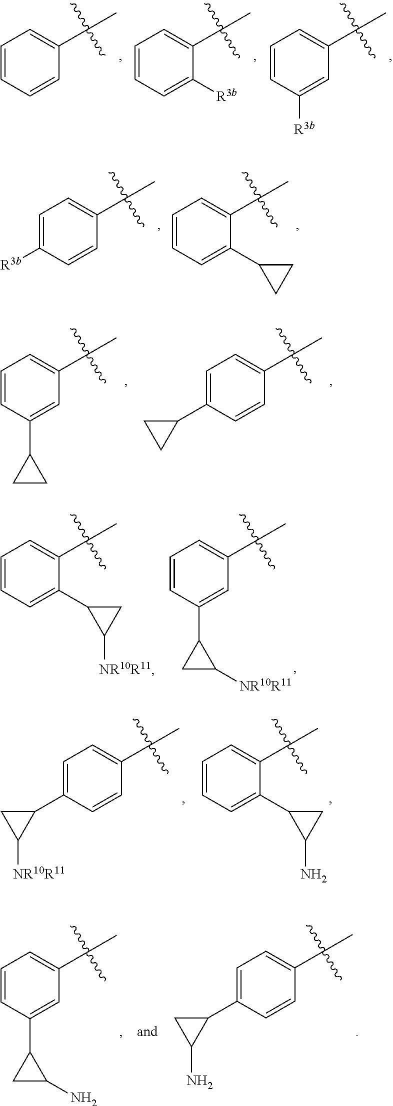

In some embodiments, R³ is C₃-C₈ cycloalkyl optionally substituted with NR¹⁰R¹¹.

In some embodiments, R³ is selected from the group consisting of

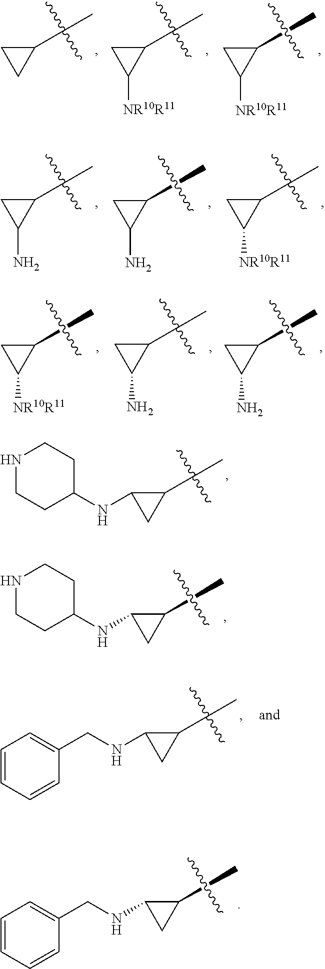

In some embodiments, R³ is (R^{3a})(R^{3b}). In some embodiments, wherein R³ is N(R^{3a})(R^{3b}), N(R^{3a})(R^{3b}) is further defined by:

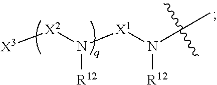

wherein each R¹² is independently selected from the group consisting of H and C₁-C₈ alkyl; wherein X¹ is selected from the group consisting of C₃-C₈ cycloalkyl, C₁-C₈ alkyl,

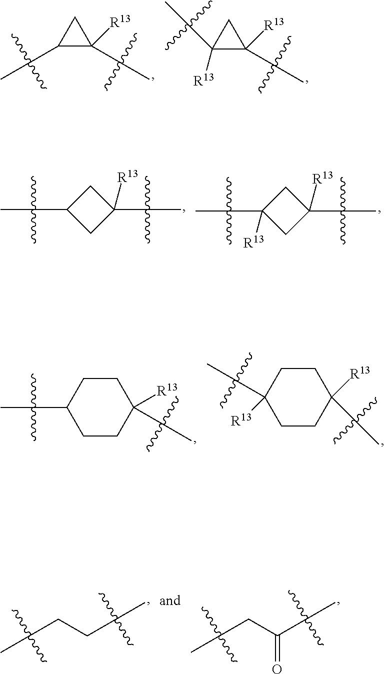

optionally wherein X¹ is substituted with one or more halogen, C₃-C₆ cycloalkyl or C₁₋₄ alkyl; wherein X² is selected from the group consisting of C₃-C₈ cycloalkyl, C₁-C₈ alkyl,

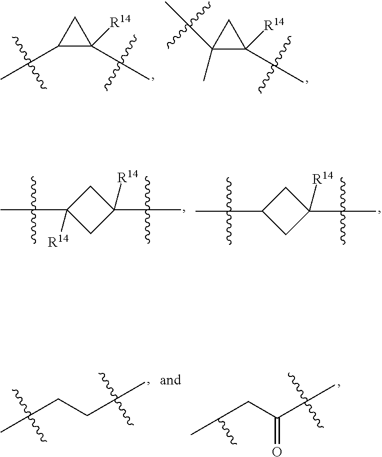

optionally wherein X² is substituted with one or more halogen, C₃-C₆ cycloalkyl or C₁₋₄ alkyl; wherein q is 0 or 1; wherein in X³ is selected from the group consisting of H, phenyl, phenyl substituted with C₁-C₄ alkyl, C₃-C₈ cycloalkyl, and C₁-C₈ alkyl, optionally wherein X³ is substituted with one or more halogen, C₃-C₆ cycloalkyl or C₁₋₄ alkyl; wherein R¹³ is independently selected from the group consisting of H, C₃-C₈ cycloalkyl, and C₁-C₈ alkyl; and wherein R¹⁴ is independently selected from the group consisting of H, C₃-C₈ cycloalkyl, and C₁-C₈ alkyl. All possible enantiomers and/or diastereomers fall within the scope of

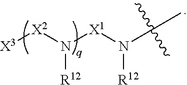

In some embodiments, q is 1; X¹ is selected from the group consisting of

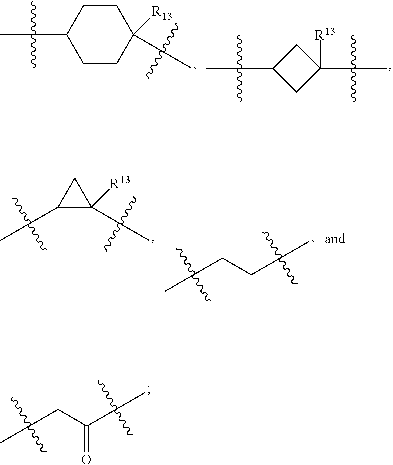

### X² is

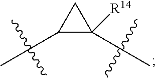

X³ is selected from the group consisting of phenyl and phenyl substituted with C₁-C₄ alkyl; R¹² is H; R¹³ is H or C₁-C₈ alkyl; and R¹⁴ is H or C₁-C₈ alkyl.

In some embodiments, q is 0; X¹ is selected from the group consisting of

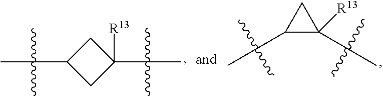

X³ is selected from the group consisting of phenyl and phenyl substituted with C₁-C₄ alkyl; R¹² is H; and R¹³ is H or C₁-C₈ alkyl.

In some embodiments, q is 0. In some embodiments, q is 1.

In some embodiments, X¹ is selected from the group consisting of

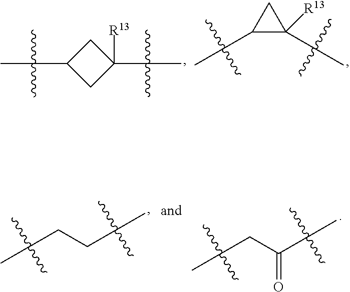

In some embodiments, X¹ is

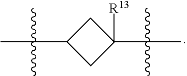

In some embodiments, X¹ is

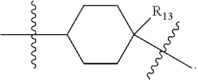

In some embodiments, X¹ is

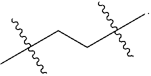 [SMILES: *CC*]

In some embodiments, X¹ is

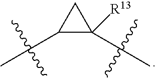

In some embodiments, X¹ is

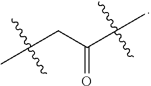 [SMILES: *CC(*)=O]

In some embodiments, X² is selected from the group consisting of

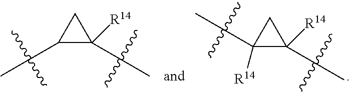

In some embodiments, X² is

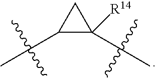

In some embodiments, X² is

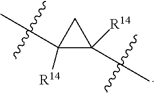

In some embodiments, X³ is selected from the group consisting of H, phenyl, and phenyl substituted with C₁-C₄ alkyl. In some embodiments, X³ is H. In some embodiments, X³ is phenyl. In some embodiments, X³ is phenyl substituted with C₁-C₄ alkyl.

In some embodiments, R¹² is independently selected from the group consisting of H, methyl, ethyl, n-propyl, isopropyl, and n-butyl. In some embodiments, R¹² is C₁-C₈ alkyl. In some embodiments, R¹² is H. In some embodiments, R¹² is methyl. In some embodiments, R¹² is ethyl. In some embodiments, R¹² is n-propyl. In some embodiments, R¹² is n-butyl.

In some embodiments, R¹³ is independently selected from the group consisting of H, methyl, ethyl, n-propyl, isopropyl, and n-butyl. In some embodiments, R¹³ is H. In some embodiments, R¹³ is C₁-C₈ alkyl. In some embodiments, R¹³ is methyl. In some embodiments, R¹³ is ethyl. In some embodiments, R¹³ is n-propyl. In some embodiments, R¹³ is n-butyl.

In some embodiments, R¹⁴ is independently selected from the group consisting of H, methyl, ethyl, n-propyl, isopropyl, and n-butyl. In some embodiments, R¹⁴ is H. In some embodiments, R¹³ is C₁-C₈ alkyl. In some embodiments, R¹⁴ is methyl. In some embodiments, R¹³ is ethyl. In some embodiments, R¹⁴ is n-propyl. In some embodiments, R¹⁴ is n-butyl.

In some embodiments, R⁴ is selected from the group consisting of H, C₁, and F.

In some embodiments, R⁴ is F.

In some embodiments, R⁴ is Cl.

In some embodiments, R⁴ is H.

In some embodiments, R⁵ is selected from the group consisting of C₁-C₈ alkyl and C₃-C₈ cycloalkyl.

In some embodiments, R⁵ is selected from the group consisting of CH₃, CH₂CH₃, CH₂CH₂CH₃, CH(CH₃)₂, CH(CH₂CH₃)₂, and cyclopropyl.

In some embodiments, R⁵ is CH₃.

In some embodiments, R⁵ is CH₂CH₃.

In some embodiments, R⁵ is CH₂CH₂CH₃.

In some embodiments, R⁵ is CH(CH₃)₂.

In some embodiments, R⁵ is cyclopropyl.

In some embodiments, R⁵ is C₁-C₈ alkyl optionally substituted with OR^{1a}.

In some embodiments, R⁵ is CH₂CH₂OH.

In some embodiments, R⁶ is H.

In some embodiments, R⁷ is selected from the group consisting of CN, tetrazolyl, CH₂O^{7a}, CO₂R^{7a}, CON(R^{7b})2, and C(═NH)—N(R^{7b})₂.

In some embodiments, R⁷ is tetrazolyl.

In some embodiments, R⁷ is selected from the group consisting of CN, CH₂OH, CO₂H, CO₂CH₃, CO₂CH₂CH₃, CONH₂, CONHOH, CONHCH₃, CON(CH₃)₂, and C(═NH)—NHOH.

In some embodiments, R⁷ is CONH₂.

In some embodiments, R⁷ is selected from the group consisting of CN and CO₂H.

In some embodiments, R⁷ is CN.

In some embodiments, R⁷ is CO₂H.

In some embodiments, L is selected from the group consisting of a bond, —(CH₂)₁₋₄—, —(CH₂)ₙN(R^{La})(CH₂)ₙ—, and -cycloalkyl-N(R^{La})—.

In some embodiments, L is selected from the group consisting of a bond, —CH₂—, —N(R^{La})CH₂—, —CH₂N(R^{La})—, -cyclopropyl-N(R^{La})—, and -cyclobutyl-N(R^{La})—.

In some embodiments, R⁸ is selected from the group consisting of C₃-C₈ cycloalkyl-N(R^{8a})(R^{8b}), N(R^{8a})—C₃-C₈ cycloalkyl-aryl, and aryl-C₃-C₈ cycloalkyl-N(R^{8a})(R^{8b}).

In some embodiments, R⁸ is selected from the group consisting of cyclopropyl-NH₂, cyclobutyl-NH₂, phenyl-cyclopropyl-NH₂, phenyl-cyclobutyl-NH₂, NH-cyclopropyl-phenyl, and NH-cyclobutyl-phenyl.

In some embodiments, the compound is selected from the compounds described in Table A and pharmaceutically acceptable salts thereof.

In some embodiments, the compound is selected from the compounds described in Table A.

TABLE A

| | |
|---|---|
| Compound I-1 | 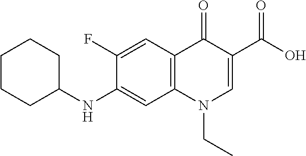 [SMILES: CCn1cc(C(=O)O)c(=O)c2cc(F)c(NC3CCCCC3)cc21] |
| Compound I-2 | 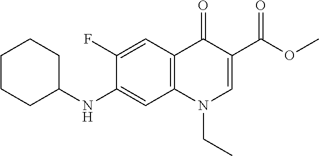 [SMILES: CCn1cc(C(=O)OC)c(=O)c2cc(F)c(NC3CCCCC3)cc21] |
| Compound I-3 | 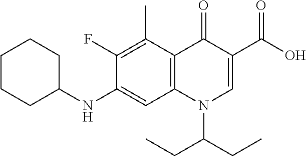 [SMILES: CCC(CC)n1cc(C(=O)O)c(=O)c2c(C)c(F)c(NC3CCCCC3)cc21] |
| Compound I-4 | 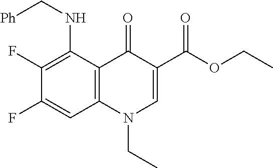 [SMILES: CCOC(=O)c1cn(CC)c2cc(F)c(F)c(NCc3ccccc3)c2c1=O] |
| Compound I-5 | 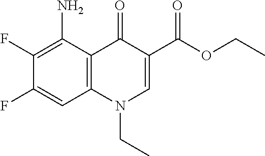 [SMILES: CCOC(=O)c1cn(CC)c2cc(F)c(F)c(N)c2c1=O] |
| Compound I-6 | 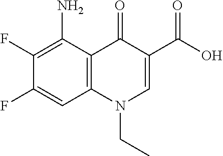 [SMILES: CCn1cc(C(=O)O)c(=O)c2c(N)c(F)c(F)cc21] |
| Compound I-7 | 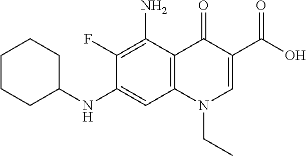 [SMILES: CCn1cc(C(=O)O)c(=O)c2c(N)c(F)c(NC3CCCCC3)cc21] |
| Compound I-8 | 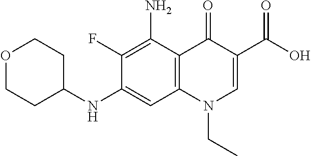 [SMILES: CCn1cc(C(=O)O)c(=O)c2c(N)c(F)c(NC3CCOCC3)cc21] |
| Compound I-9 | 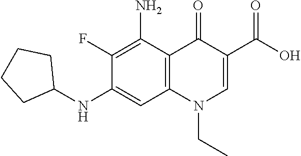 [SMILES: CCn1cc(C(=O)O)c(=O)c2c(N)c(F)c(NC3CCCC3)cc21] |
| Compound I-10 | 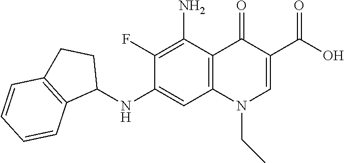 [SMILES: CCn1cc(C(=O)O)c(=O)c2c(N)c(F)c(NC3CCc4ccccc43)cc21] |
| Compound I-11 | 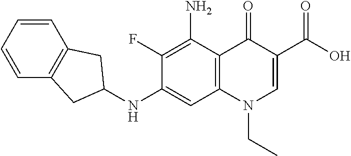 [SMILES: CCn1cc(C(=O)O)c(=O)c2c(N)c(F)c(NC3Cc4ccccc4C3)cc21] |
| Compound I-12 | 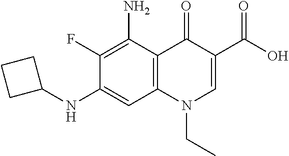 [SMILES: CCn1cc(C(=O)O)c(=O)c2c(N)c(F)c(NC3CCC3)cc21] |
| Compound I-13A | 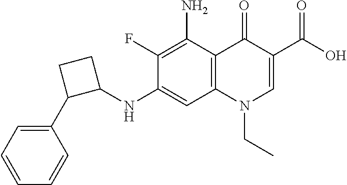 [SMILES: CCn1cc(C(=O)O)c(=O)c2c(N)c(F)c(NC3CCC3c3ccccc3)cc21] |
| Compound I-13 | 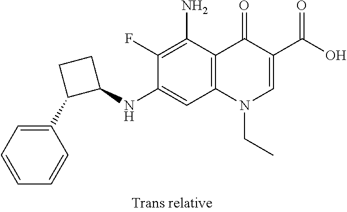 [SMILES: CCn1cc(C(=O)O)c(=O)c2c(N)c(F)c(N[C@@H]3CC[C@H]3c3ccccc3)cc21] |
| Compound I-14 | 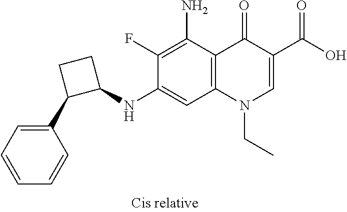 [SMILES: CCn1cc(C(=O)O)c(=O)c2c(N)c(F)c(N[C@@H]3CC[C@@H]3c3ccccc3)cc21] |
| Compound I-15 | 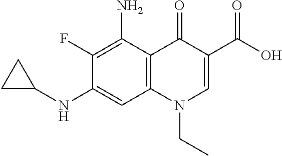 [SMILES: CCn1cc(C(=O)O)c(=O)c2c(N)c(F)c(NC3CC3)cc21] |
| Compound I-16 | 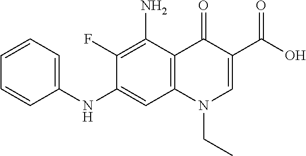 [SMILES: CCn1cc(C(=O)O)c(=O)c2c(N)c(F)c(Nc3ccccc3)cc21] |
| Compound I-17 | 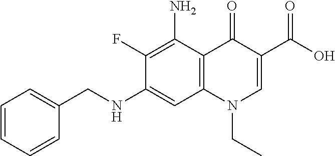 [SMILES: CCn1cc(C(=O)O)c(=O)c2c(N)c(F)c(NCc3ccccc3)cc21] |
| Compound I-18 | 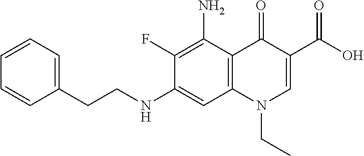 [SMILES: CCn1cc(C(=O)O)c(=O)c2c(N)c(F)c(NCCc3ccccc3)cc21] |
| Compound I-19 | 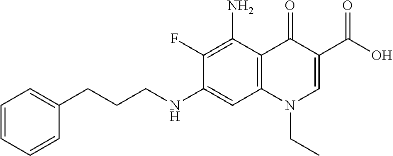 [SMILES: CCn1cc(C(=O)O)c(=O)c2c(N)c(F)c(NCCCc3ccccc3)cc21] |
| Compound I-20A | 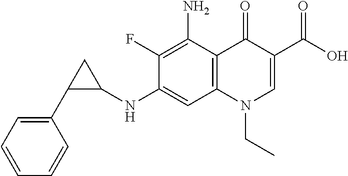 [SMILES: CCn1cc(C(=O)O)c(=O)c2c(N)c(F)c(NC3CC3c3ccccc3)cc21] |
| Compound I-20 | 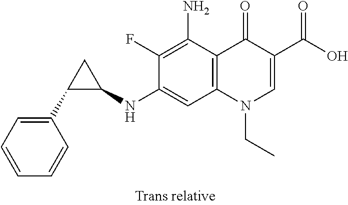 [SMILES: CCn1cc(C(=O)O)c(=O)c2c(N)c(F)c(N[C@@H]3C[C@H]3c3ccccc3)cc21] |
| Compound I-21 | 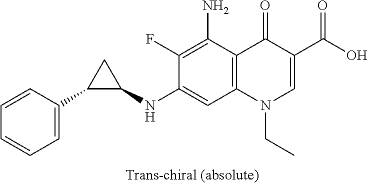 [SMILES: CCn1cc(C(=O)O)c(=O)c2c(N)c(F)c(N[C@@H]3C[C@H]3c3ccccc3)cc21] |
| Compound I-22 | 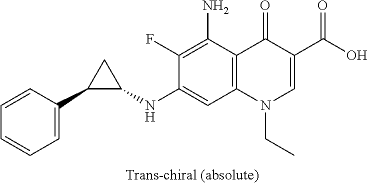 [SMILES: CCn1cc(C(=O)O)c(=O)c2c(N)c(F)c(N[C@H]3C[C@@H]3c3ccccc3)cc21] |
| Compound I-23 | 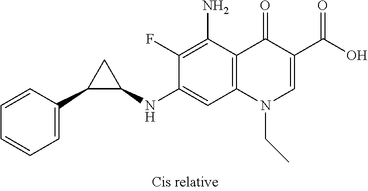 [SMILES: CCn1cc(C(=O)O)c(=O)c2c(N)c(F)c(N[C@@H]3C[C@@H]3c3ccccc3)cc21] |
| Compound I-24 | 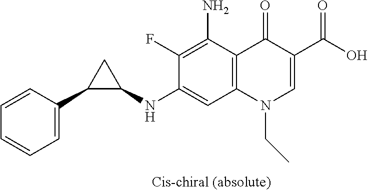 [SMILES: CCn1cc(C(=O)O)c(=O)c2c(N)c(F)c(N[C@@H]3C[C@@H]3c3ccccc3)cc21] |
| Compound I-25 | 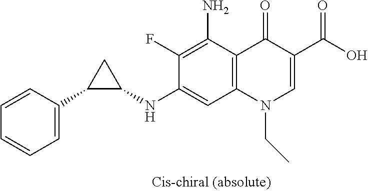 [SMILES: CCn1cc(C(=O)O)c(=O)c2c(N)c(F)c(N[C@H]3C[C@H]3c3ccccc3)cc21] |
| Compound I-26A | 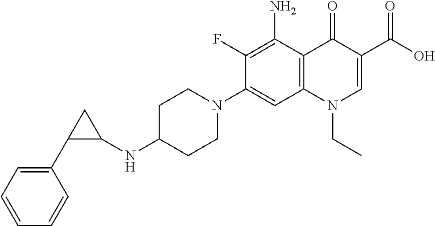 [SMILES: CCn1cc(C(=O)O)c(=O)c2c(N)c(F)c(N3CCC(NC4CC4c4ccccc4)CC3)cc21] |
| Compound I-26 | 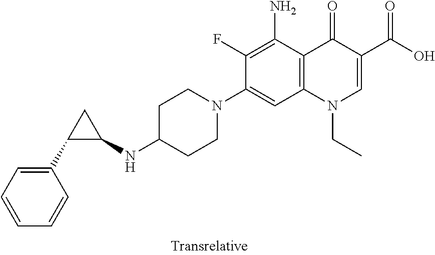 [SMILES: CCn1cc(C(=O)O)c(=O)c2c(N)c(F)c(N3CCC(N[C@@H]4C[C@H]4c4ccccc4)CC3)cc21] |
| Compound I-27A | 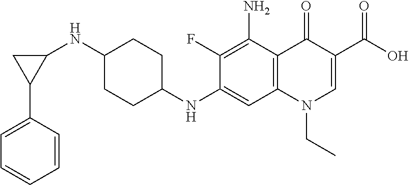 [SMILES: CCn1cc(C(=O)O)c(=O)c2c(N)c(F)c(NC3CCC(NC4CC4c4ccccc4)CC3)cc21] |
| Compound I-27 | 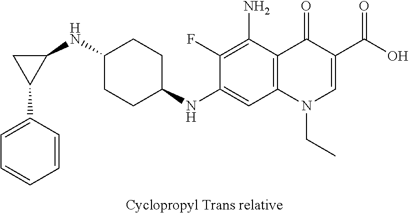 [SMILES: CCn1cc(C(=O)O)c(=O)c2c(N)c(F)c(N[C@H]3CC[C@H](N[C@@H]4C[C@H]4c4ccccc4)CC3)cc21] |
| Compound I-28 | 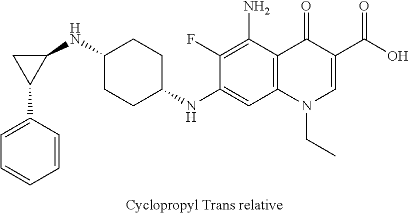 [SMILES: CCn1cc(C(=O)O)c(=O)c2c(N)c(F)c(N[C@H]3CC[C@@H](N[C@@H]4C[C@H]4c4ccccc4)CC3)cc21] |
| Compound I-29A | 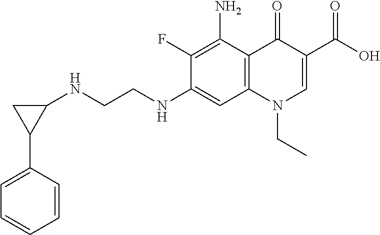 [SMILES: CCn1cc(C(=O)O)c(=O)c2c(N)c(F)c(NCCNC3CC3c3ccccc3)cc21] |
| Compound I-29 | 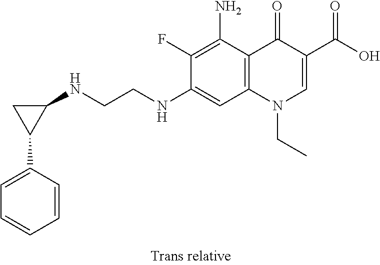 [SMILES: CCn1cc(C(=O)O)c(=O)c2c(N)c(F)c(NCCN[C@@H]3C[C@H]3c3ccccc3)cc21] |
| Compound I-30A | 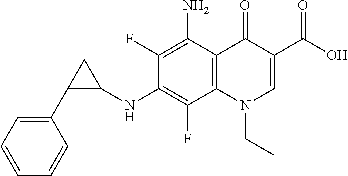 [SMILES: CCn1cc(C(=O)O)c(=O)c2c(N)c(F)c(NC3CC3c3ccccc3)c(F)c21] |
| Compound I-30 | 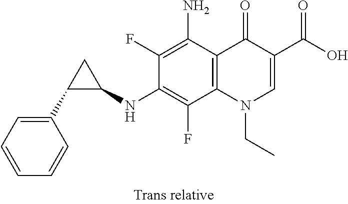 [SMILES: CCn1cc(C(=O)O)c(=O)c2c(N)c(F)c(N[C@@H]3C[C@H]3c3ccccc3)c(F)c21] |
| Compound I-31A | 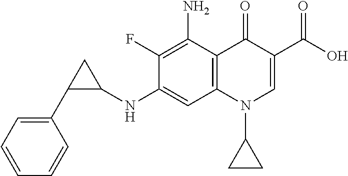 [SMILES: Nc1c(F)c(NC2CC2c2ccccc2)cc2c1c(=O)c(C(=O)O)cn2C1CC1] |
| Compound I-31 | 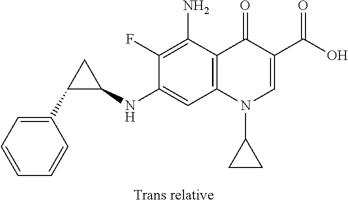 [SMILES: Nc1c(F)c(N[C@@H]2C[C@H]2c2ccccc2)cc2c1c(=O)c(C(=O)O)cn2C1CC1] |
| Compound I-32A | 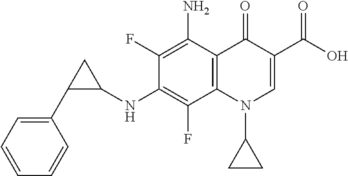 [SMILES: Nc1c(F)c(NC2CC2c2ccccc2)c(F)c2c1c(=O)c(C(=O)O)cn2C1CC1] |
| Compound I-32 | 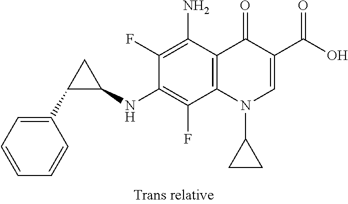 [SMILES: Nc1c(F)c(N[C@@H]2C[C@H]2c2ccccc2)c(F)c2c1c(=O)c(C(=O)O)cn2C1CC1] |
| Compound I-33A | 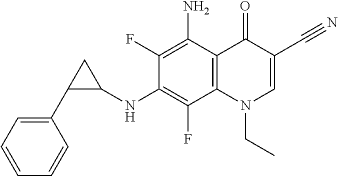 [SMILES: CCn1cc(C#N)c(=O)c2c(N)c(F)c(NC3CC3c3ccccc3)c(F)c21] |
| Compound I-33 | 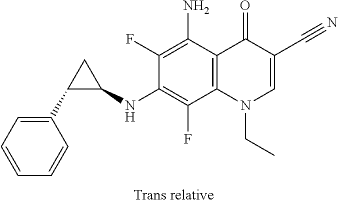 [SMILES: CCn1cc(C#N)c(=O)c2c(N)c(F)c(N[C@@H]3C[C@H]3c3ccccc3)c(F)c21] |
| Compound I-34A | 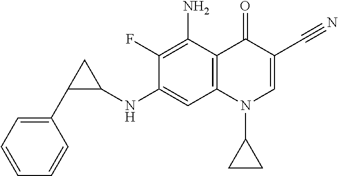 [SMILES: N#Cc1cn(C2CC2)c2cc(NC3CC3c3ccccc3)c(F)c(N)c2c1=O] |
| Compound I-34 | 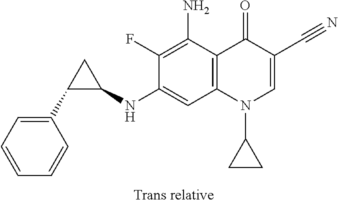 [SMILES: N#Cc1cn(C2CC2)c2cc(N[C@@H]3C[C@H]3c3ccccc3)c(F)c(N)c2c1=O] |
| Compound I-35A | 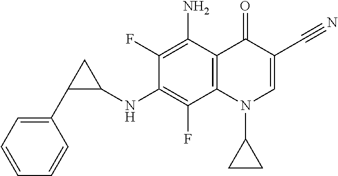 [SMILES: N#Cc1cn(C2CC2)c2c(F)c(NC3CC3c3ccccc3)c(F)c(N)c2c1=O] |
| Compound I-35 | 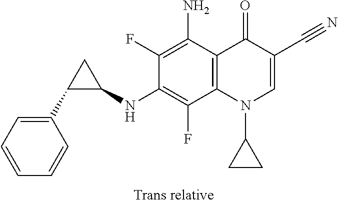 [SMILES: N#Cc1cn(C2CC2)c2c(F)c(N[C@@H]3C[C@H]3c3ccccc3)c(F)c(N)c2c1=O] |
| Compound I-36A | 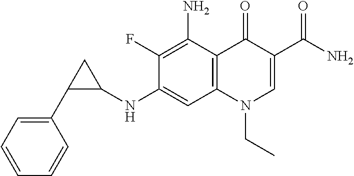 [SMILES: CCn1cc(C(N)=O)c(=O)c2c(N)c(F)c(NC3CC3c3ccccc3)cc21] |
| Compound I-36 | 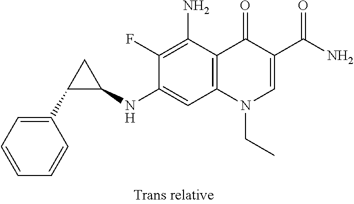 [SMILES: CCn1cc(C(N)=O)c(=O)c2c(N)c(F)c(N[C@@H]3C[C@H]3c3ccccc3)cc21] |
| Compound I-37A | 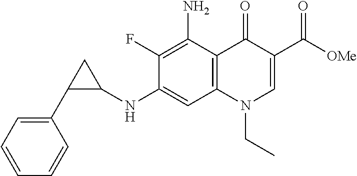 [SMILES: CCn1cc(C(=O)OC)c(=O)c2c(N)c(F)c(NC3CC3c3ccccc3)cc21] |
| Compound I-37 | 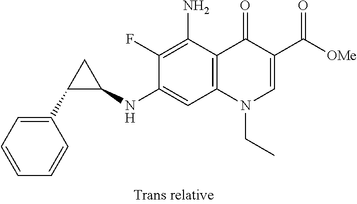 [SMILES: CCn1cc(C(=O)OC)c(=O)c2c(N)c(F)c(N[C@@H]3C[C@H]3c3ccccc3)cc21] |
| Compound I-38A | 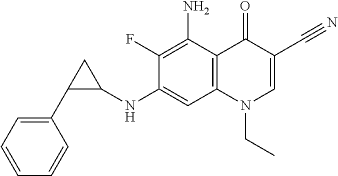 [SMILES: CCn1cc(C#N)c(=O)c2c(N)c(F)c(NC3CC3c3ccccc3)cc21] |
| Compound I-38 | 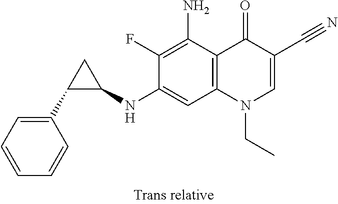 [SMILES: CCn1cc(C#N)c(=O)c2c(N)c(F)c(N[C@@H]3C[C@H]3c3ccccc3)cc21] |
| Compound I-39A | 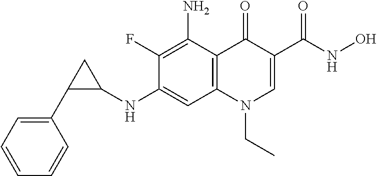 [SMILES: CCn1cc(C(=O)NO)c(=O)c2c(N)c(F)c(NC3CC3c3ccccc3)cc21] |
| Compound I-39 | 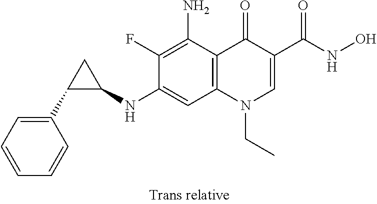 [SMILES: CCn1cc(C(=O)NO)c(=O)c2c(N)c(F)c(N[C@@H]3C[C@H]3c3ccccc3)cc21] |
| Compound I-40A | 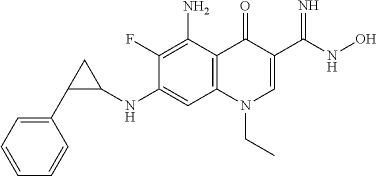 [SMILES: CCn1cc(C(=N)NO)c(=O)c2c(N)c(F)c(NC3CC3c3ccccc3)cc21] |
| Compound I-40 | 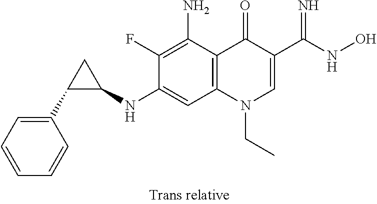 [SMILES: CCn1cc(C(=N)NO)c(=O)c2c(N)c(F)c(N[C@@H]3C[C@H]3c3ccccc3)cc21] |
| Compound I-41A | 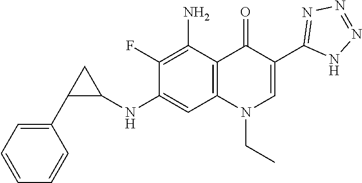 [SMILES: CCn1cc(-c2nnn[nH]2)c(=O)c2c(N)c(F)c(NC3CC3c3ccccc3)cc21] |
| Compound I-41 | 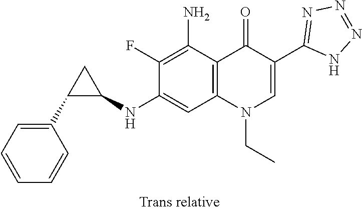 [SMILES: CCn1cc(-c2nnn[nH]2)c(=O)c2c(N)c(F)c(N[C@@H]3C[C@H]3c3ccccc3)cc21] |
| Compound I-42A | 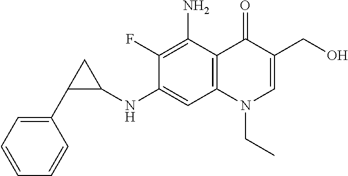 [SMILES: CCn1cc(CO)c(=O)c2c(N)c(F)c(NC3CC3c3ccccc3)cc21] |
| Compound I-42 | 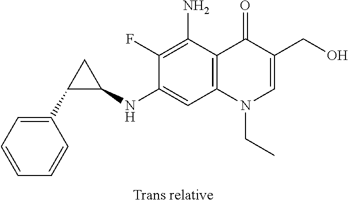 [SMILES: CCn1cc(CO)c(=O)c2c(N)c(F)c(N[C@@H]3C[C@H]3c3ccccc3)cc21] |
| Compound I-43A | 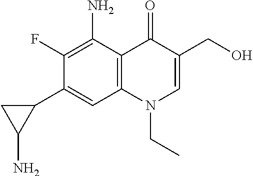 [SMILES: CCn1cc(CO)c(=O)c2c(N)c(F)c(C3CC3N)cc21] |
| Compound I-43 | 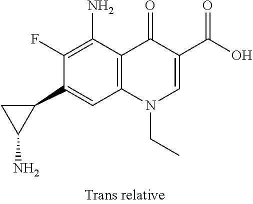 [SMILES: CCn1cc(C(=O)O)c(=O)c2c(N)c(F)c([C@@H]3C[C@H]3N)cc21] |
| Compound I-44A | 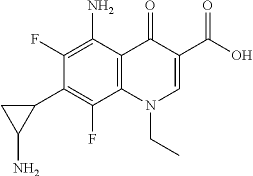 [SMILES: CCn1cc(C(=O)O)c(=O)c2c(N)c(F)c(C3CC3N)c(F)c21] |
| Compound I-44 | 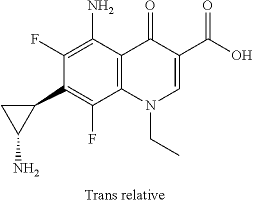 [SMILES: CCn1cc(C(=O)O)c(=O)c2c(N)c(F)c([C@@H]3C[C@H]3N)c(F)c21] |
| Compound I-45A | 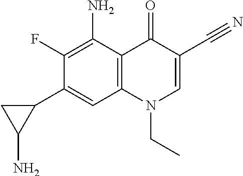 [SMILES: CCn1cc(C#N)c(=O)c2c(N)c(F)c(C3CC3N)cc21] |
| Compound I-45 | 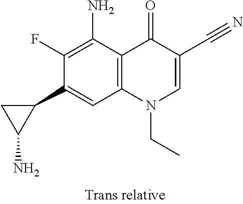 [SMILES: CCn1cc(C#N)c(=O)c2c(N)c(F)c([C@@H]3C[C@H]3N)cc21] |
| Compound I-46A | 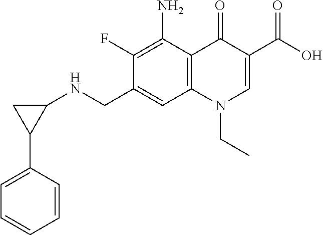 [SMILES: CCn1cc(C(=O)O)c(=O)c2c(N)c(F)c(CNC3CC3c3ccccc3)cc21] |
| Compound I-46 | 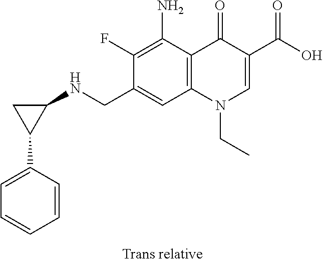 [SMILES: CCn1cc(C(=O)O)c(=O)c2c(N)c(F)c(CN[C@@H]3C[C@H]3c3ccccc3)cc21] |
| Compound I-47A | 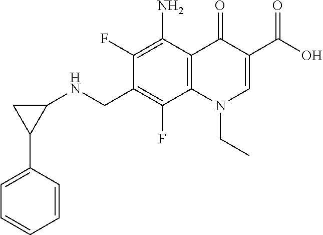 [SMILES: CCn1cc(C(=O)O)c(=O)c2c(N)c(F)c(CNC3CC3c3ccccc3)c(F)c21] |
| Compound I-47 | 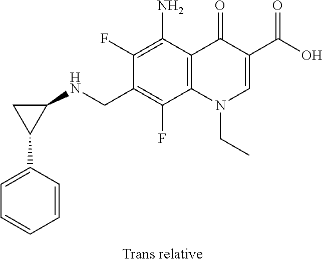 [SMILES: CCn1cc(C(=O)O)c(=O)c2c(N)c(F)c(CN[C@@H]3C[C@H]3c3ccccc3)c(F)c21] |
| Compound I-48A | 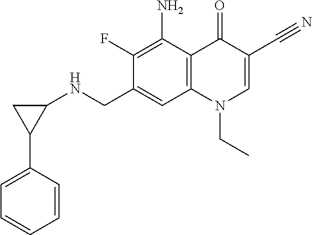 [SMILES: CCn1cc(C#N)c(=O)c2c(N)c(F)c(CNC3CC3c3ccccc3)cc21] |
| Compound I-48 | 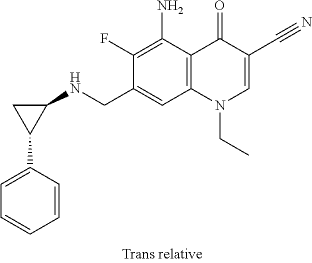 [SMILES: CCn1cc(C#N)c(=O)c2c(N)c(F)c(CN[C@@H]3C[C@H]3c3ccccc3)cc21] |
| Compound I-49A | 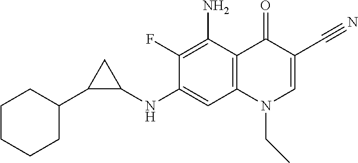 [SMILES: CCn1cc(C#N)c(=O)c2c(N)c(F)c(NC3CC3C3CCCCC3)cc21] |
| Compound I-49 | 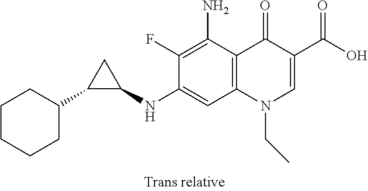 [SMILES: CCn1cc(C(=O)O)c(=O)c2c(N)c(F)c(N[C@@H]3C[C@H]3C3CCCCC3)cc21] |
| Compound I-50 | 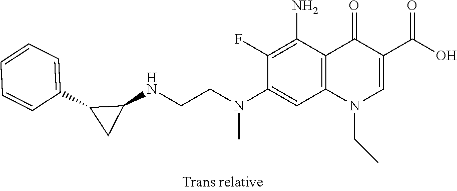 [SMILES: CCn1cc(C(=O)O)c(=O)c2c(N)c(F)c(N(C)CCN[C@H]3C[C@@H]3c3ccccc3)cc21] |
| Compound I-51 | 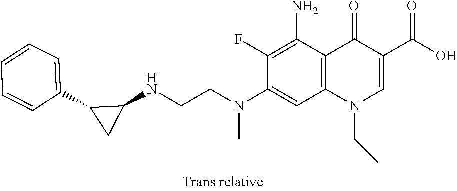 [SMILES: CCn1cc(C(=O)O)c(=O)c2c(N)c(F)c(N(C)CCN[C@H]3C[C@@H]3c3ccccc3)cc21] |
| Compound I-52 | 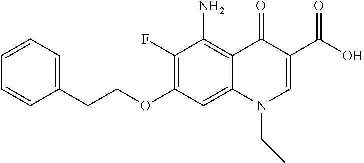 [SMILES: CCn1cc(C(=O)O)c(=O)c2c(N)c(F)c(OCCc3ccccc3)cc21] |
| Compound I-53 | 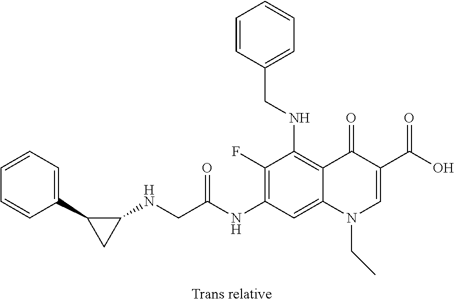 [SMILES: CCn1cc(C(=O)O)c(=O)c2c(NCc3ccccc3)c(F)c(NC(=O)CN[C@@H]3C[C@H]3c3ccccc3)cc21] |
| Compound I-54 | 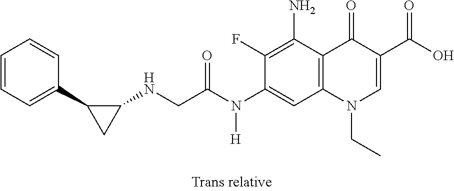 [SMILES: CCn1cc(C(=O)O)c(=O)c2c(N)c(F)c(NC(=O)CN[C@@H]3C[C@H]3c3ccccc3)cc21] |
| Compound I-55 | 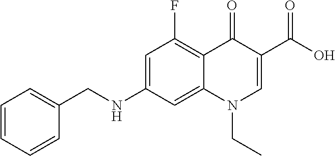 [SMILES: CCn1cc(C(=O)O)c(=O)c2c(F)cc(NCc3ccccc3)cc21] |
| Compound I-56 | 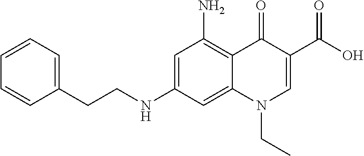 [SMILES: CCn1cc(C(=O)O)c(=O)c2c(N)cc(NCCc3ccccc3)cc21] |
| Compound I-57 | 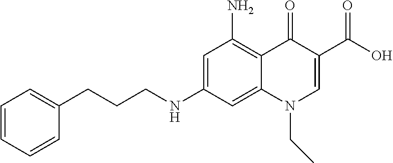 [SMILES: CCn1cc(C(=O)O)c(=O)c2c(N)cc(NCCCc3ccccc3)cc21] |
| Compound I-58 | 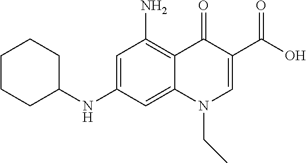 [SMILES: CCn1cc(C(=O)O)c(=O)c2c(N)cc(NC3CCCCC3)cc21] |
| Compound I-59 | 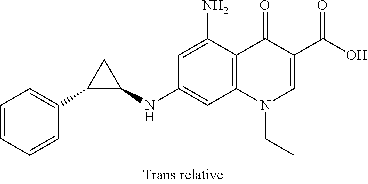 [SMILES: CCn1cc(C(=O)O)c(=O)c2c(N)cc(N[C@@H]3C[C@H]3c3ccccc3)cc21] |
| Compound I-60 | 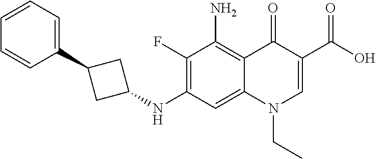 [SMILES: CCn1cc(C(=O)O)c(=O)c2c(N)c(F)c(N[C@H]3C[C@H](c4ccccc4)C3)cc21] |
| Compound I-61 | 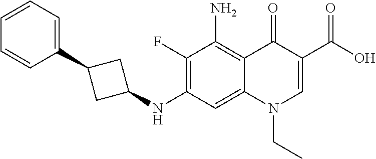 [SMILES: CCn1cc(C(=O)O)c(=O)c2c(N)c(F)c(N[C@H]3C[C@@H](c4ccccc4)C3)cc21] |
| Compound I-62 | 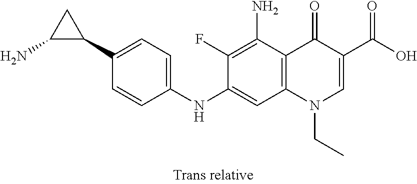 [SMILES: CCn1cc(C(=O)O)c(=O)c2c(N)c(F)c(Nc3ccc([C@@H]4C[C@H]4N)cc3)cc21] |
| Compound I-63 | 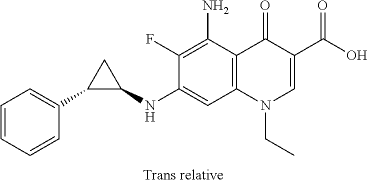 [SMILES: CCn1cc(C(=O)O)c(=O)c2c(N)c(F)c(N[C@@H]3C[C@H]3c3ccccc3)cc21] |
| Compound I-64 | 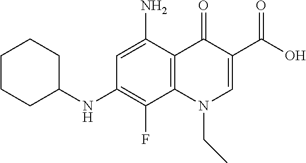 [SMILES: CCn1cc(C(=O)O)c(=O)c2c(N)cc(NC3CCCCC3)c(F)c21] |
| Compound I-65 | 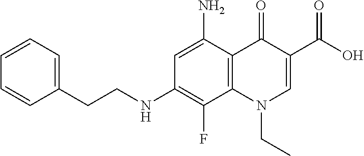 [SMILES: CCn1cc(C(=O)O)c(=O)c2c(N)cc(NCCc3ccccc3)c(F)c21] |
| Compound I-66 | 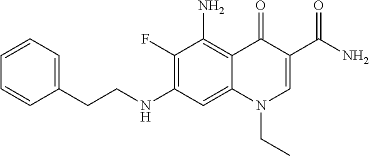 [SMILES: CCn1cc(C(N)=O)c(=O)c2c(N)c(F)c(NCCc3ccccc3)cc21] |
| Compound I-67 | 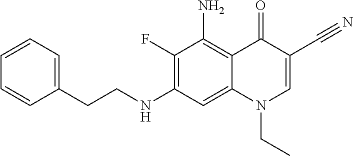 [SMILES: CCn1cc(C#N)c(=O)c2c(N)c(F)c(NCCc3ccccc3)cc21] |
| Compound I-68 | 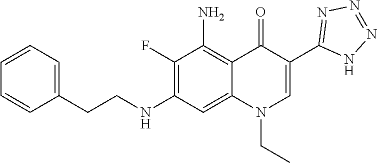 [SMILES: CCn1cc(-c2nnn[nH]2)c(=O)c2c(N)c(F)c(NCCc3ccccc3)cc21] |
| Compound I-69 | 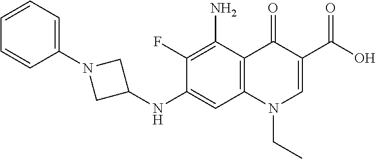 [SMILES: CCn1cc(C(=O)O)c(=O)c2c(N)c(F)c(NC3CN(c4ccccc4)C3)cc21] |
| Compound I-70 | 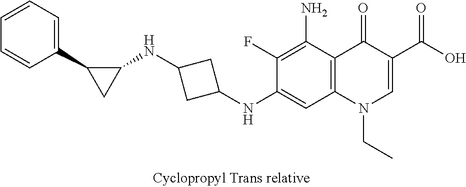 [SMILES: CCn1cc(C(=O)O)c(=O)c2c(N)c(F)c(NC3CC(N[C@@H]4C[C@H]4c4ccccc4)C3)cc21] |
| Compound I-71 | 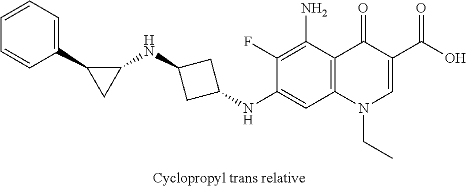 [SMILES: CCn1cc(C(=O)O)c(=O)c2c(N)c(F)c(N[C@H]3C[C@H](N[C@@H]4C[C@H]4c4ccccc4)C3)cc21] |
| Compound I-72 | 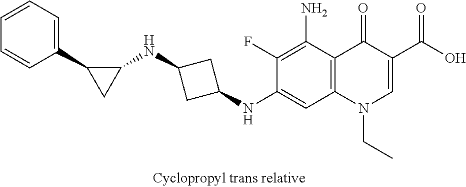 [SMILES: CCn1cc(C(=O)O)c(=O)c2c(N)c(F)c(N[C@H]3C[C@@H](N[C@@H]4C[C@H]4c4ccccc4)C3)cc21] |
| Compound I-73 | 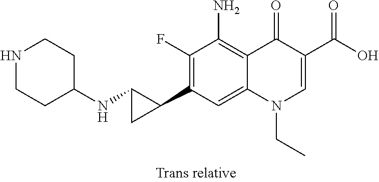 [SMILES: CCn1cc(C(=O)O)c(=O)c2c(N)c(F)c([C@H]3C[C@@H]3NC3CCNCC3)cc21] |
| Compound I-74 | 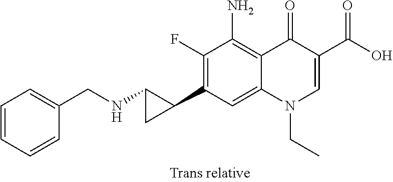 [SMILES: CCn1cc(C(=O)O)c(=O)c2c(N)c(F)c([C@H]3C[C@@H]3NCc3ccccc3)cc21] |
| Compound I-75 | 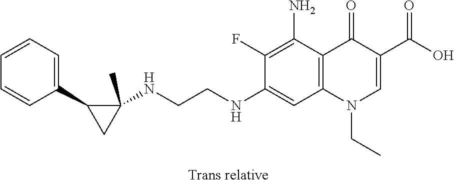 [SMILES: CCn1cc(C(=O)O)c(=O)c2c(N)c(F)c(NCCN[C@]3(C)C[C@H]3c3ccccc3)cc21] |
| Compound I-76 | 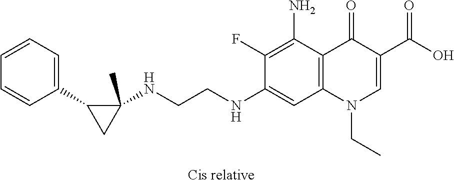 [SMILES: CCn1cc(C(=O)O)c(=O)c2c(N)c(F)c(NCCN[C@]3(C)C[C@@H]3c3ccccc3)cc21] |
| Compound I-77 | 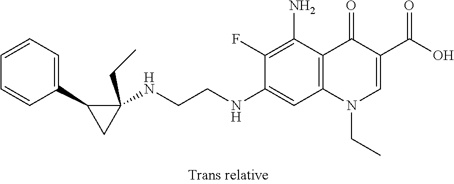 [SMILES: CCn1cc(C(=O)O)c(=O)c2c(N)c(F)c(NCCN[C@]3(CC)C[C@H]3c3ccccc3)cc21] |
| Compound I-78 | 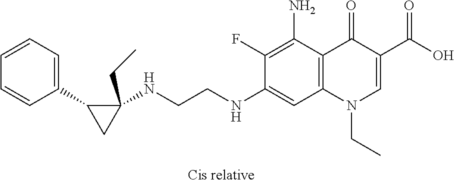 [SMILES: CCn1cc(C(=O)O)c(=O)c2c(N)c(F)c(NCCN[C@]3(CC)C[C@@H]3c3ccccc3)cc21] |
| Compound I-79 | 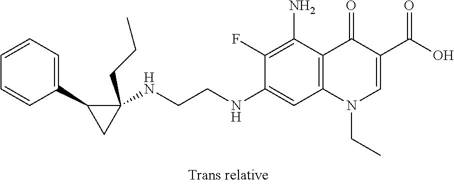 [SMILES: CCC[C@@]1(NCCNc2cc3c(c(N)c2F)c(=O)c(C(=O)O)cn3CC)C[C@H]1c1ccccc1] |
| Compound I-80 | 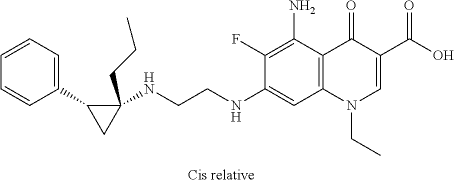 [SMILES: CCC[C@@]1(NCCNc2cc3c(c(N)c2F)c(=O)c(C(=O)O)cn3CC)C[C@@H]1c1ccccc1] |
| Compound I-81 | 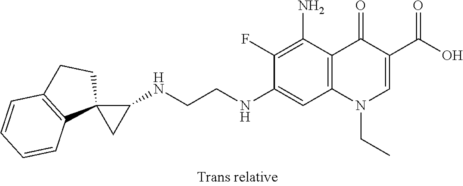 [SMILES: CCn1cc(C(=O)O)c(=O)c2c(N)c(F)c(NCCN[C@@H]3C[C@]34CCc3ccccc34)cc21] |
| Compound I-82 | 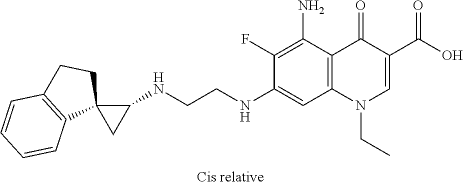 [SMILES: CCn1cc(C(=O)O)c(=O)c2c(N)c(F)c(NCCN[C@@H]3C[C@@]34CCc3ccccc34)cc21] |
| Compound I-83 | 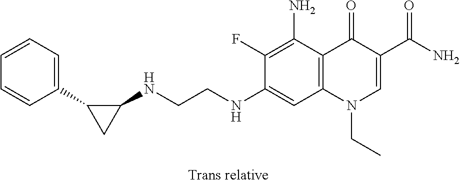 [SMILES: CCn1cc(C(N)=O)c(=O)c2c(N)c(F)c(NCCN[C@H]3C[C@@H]3c3ccccc3)cc21] |
| Compound I-84 | 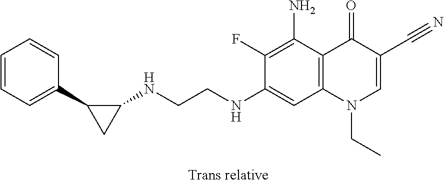 [SMILES: CCn1cc(C#N)c(=O)c2c(N)c(F)c(NCCN[C@@H]3C[C@H]3c3ccccc3)cc21] |
| Compound I-85 | 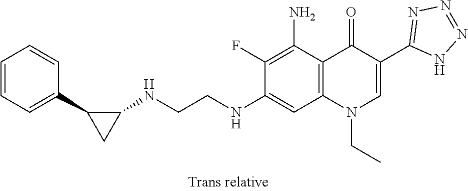 [SMILES: CCn1cc(-c2nnn[nH]2)c(=O)c2c(N)c(F)c(NCCN[C@@H]3C[C@H]3c3ccccc3)cc21] |
| Compound I-86 | 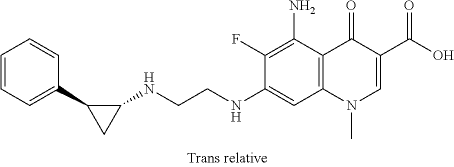 [SMILES: Cn1cc(C(=O)O)c(=O)c2c(N)c(F)c(NCCN[C@@H]3C[C@H]3c3ccccc3)cc21] |
| Compound I-87 | 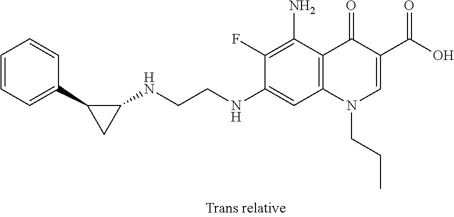 [SMILES: CCCn1cc(C(=O)O)c(=O)c2c(N)c(F)c(NCCN[C@@H]3C[C@H]3c3ccccc3)cc21] |
| Compound I-88 | 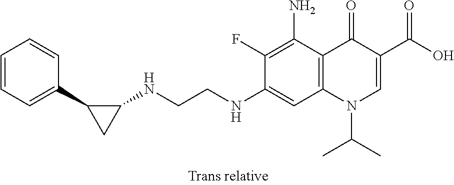 [SMILES: CC(C)n1cc(C(=O)O)c(=O)c2c(N)c(F)c(NCCN[C@@H]3C[C@H]3c3ccccc3)cc21] |
| Compound I-89 | 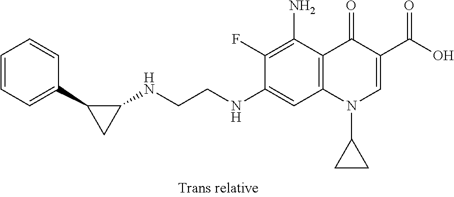 [SMILES: Nc1c(F)c(NCCN[C@@H]2C[C@H]2c2ccccc2)cc2c1c(=O)c(C(=O)O)cn2C1CC1] |
| Compound I-90 | 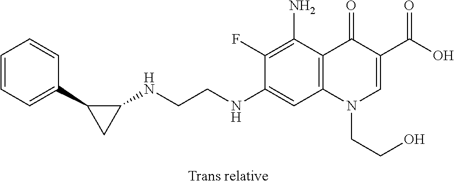 [SMILES: Nc1c(F)c(NCCN[C@@H]2C[C@H]2c2ccccc2)cc2c1c(=O)c(C(=O)O)cn2CCO] |
| Compound I-91 | 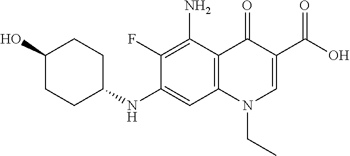 [SMILES: CCn1cc(C(=O)O)c(=O)c2c(N)c(F)c(N[C@H]3CC[C@H](O)CC3)cc21] |
| Compound I-92 | 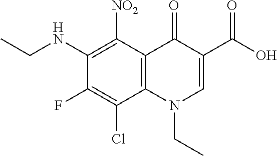 [SMILES: CCNc1c(F)c(Cl)c2c(c1[N+](=O)[O-])c(=O)c(C(=O)O)cn2CC] |
| Compound I-93 | 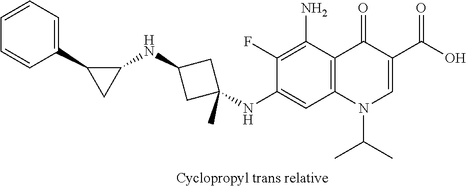 [SMILES: CC(C)n1cc(C(=O)O)c(=O)c2c(N)c(F)c(N[C@]3(C)C[C@@H](N[C@@H]4C[C@H]4c4ccccc4)C3)cc21] |
| Compound I-94 | 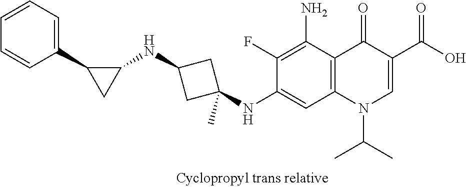 [SMILES: CC(C)n1cc(C(=O)O)c(=O)c2c(N)c(F)c(N[C@]3(C)C[C@H](N[C@@H]4C[C@H]4c4ccccc4)C3)cc21] |
| Compound I-95 | 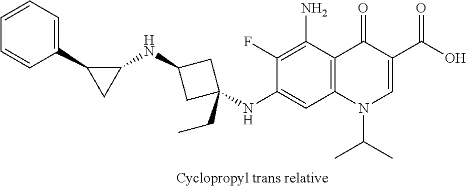 [SMILES: CC[C@]1(Nc2cc3c(c(N)c2F)c(=O)c(C(=O)O)cn3C(C)C)C[C@H](N[C@@H]2C[C@H]2c2ccccc2)C1] |
| Compound I-96 | 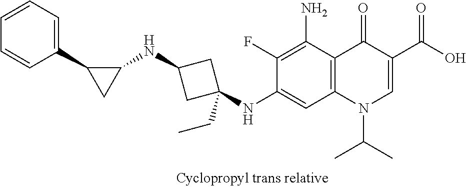 [SMILES: CC[C@]1(Nc2cc3c(c(N)c2F)c(=O)c(C(=O)O)cn3C(C)C)C[C@@H](N[C@@H]2C[C@H]2c2ccccc2)C1] |
| Compound I-97 | 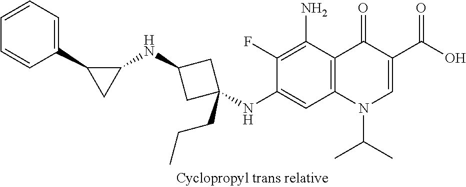 [SMILES: CCC[C@]1(Nc2cc3c(c(N)c2F)c(=O)c(C(=O)O)cn3C(C)C)C[C@H](N[C@@H]2C[C@H]2c2ccccc2)C1] |
| Compound I-98 | 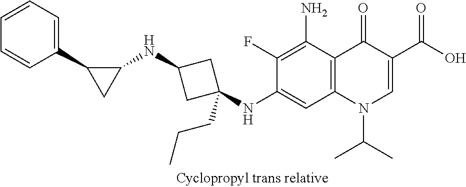 [SMILES: CCC[C@]1(Nc2cc3c(c(N)c2F)c(=O)c(C(=O)O)cn3C(C)C)C[C@@H](N[C@@H]2C[C@H]2c2ccccc2)C1] |
| Compound I-99 | 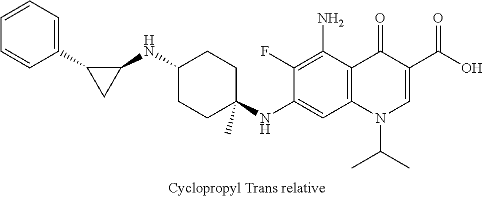 [SMILES: CC(C)n1cc(C(=O)O)c(=O)c2c(N)c(F)c(N[C@]3(C)CC[C@@H](N[C@H]4C[C@@H]4c4ccccc4)CC3)cc21] |
| Compound I-100 | 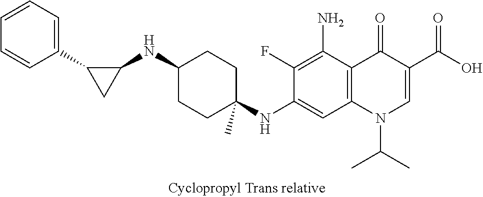 [SMILES: CC(C)n1cc(C(=O)O)c(=O)c2c(N)c(F)c(N[C@]3(C)CC[C@H](N[C@H]4C[C@@H]4c4ccccc4)CC3)cc21] |
| Compound I-101 | 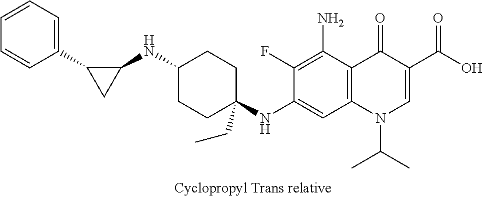 [SMILES: CC[C@]1(Nc2cc3c(c(N)c2F)c(=O)c(C(=O)O)cn3C(C)C)CC[C@H](N[C@H]2C[C@@H]2c2ccccc2)CC1] |
| Compound I-102 | 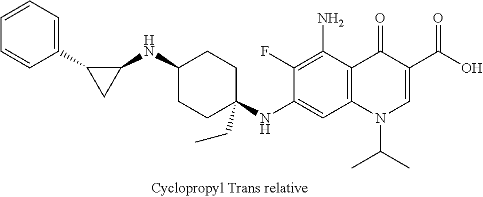 [SMILES: CC[C@]1(Nc2cc3c(c(N)c2F)c(=O)c(C(=O)O)cn3C(C)C)CC[C@@H](N[C@H]2C[C@@H]2c2ccccc2)CC1] |
| Compound I-103 | 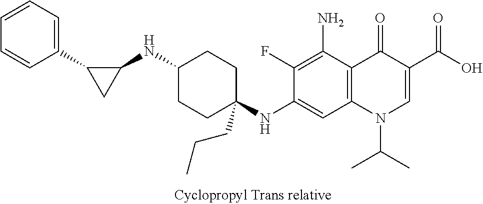 [SMILES: CCC[C@]1(Nc2cc3c(c(N)c2F)c(=O)c(C(=O)O)cn3C(C)C)CC[C@H](N[C@H]2C[C@@H]2c2ccccc2)CC1] |
| Compound I-104 | 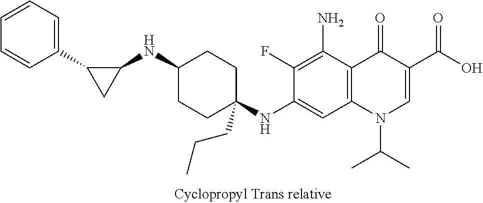 [SMILES: CCC[C@]1(Nc2cc3c(c(N)c2F)c(=O)c(C(=O)O)cn3C(C)C)CC[C@@H](N[C@H]2C[C@@H]2c2ccccc2)CC1] |
| Compound I-105 | 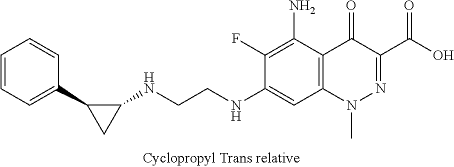 [SMILES: Cn1nc(C(=O)O)c(=O)c2c(N)c(F)c(NCCN[C@@H]3C[C@H]3c3ccccc3)cc21] |

It is understood that, when an enantiomer of a compound is shown in Table A, both enantiomers of the compound are intended to be encompassed by Table A.

In some embodiments, the compound is not Compound I-3.

In some embodiments, the compound is not Compound I-3 or any pharmaceutically acceptable salt thereof.

In some embodiments, the compound is not Compound I-7.

In some embodiments, the compound is not Compound I-7 or any pharmaceutically acceptable salt thereof.

In some embodiments, the compound is not Compound I-3 or Compound I-7.

In some embodiments, the compound is not Compound I-3, Compound I-7, or any pharmaceutically acceptable salt thereof.

In some embodiments, the compound has one, two, three, or more of the following features:

a) Q¹ is CR⁶;

b) R¹ is H, NH₂, or CH₃.

c) R² is F;

d) R⁴ is H, Cl, or F;

e) R⁵ is CH₂CH₃ or cyclopropyl;

f) R⁶ is H;

g) R⁷ is CN or CO₂H;

e) R³ is

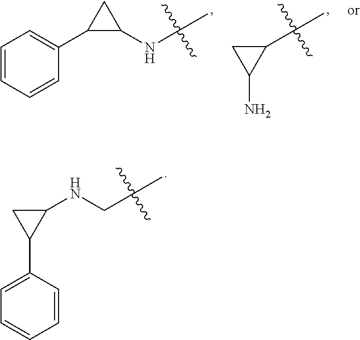 [SMILES: *NC1CC1c1ccccc1 ; *C1CC1N ; *CNC1CC1c1ccccc1]

In some embodiments, the compound has one, two, three, or more of the following features:

a) Q¹ is CR⁶;

b) R¹ is NH₂.

c) R² is F;

d) R⁴ is H;

e) R⁵ is CH₂CH₃;

f) R⁶ is H;

g) R⁷ is CO₂H;

e) R³ is

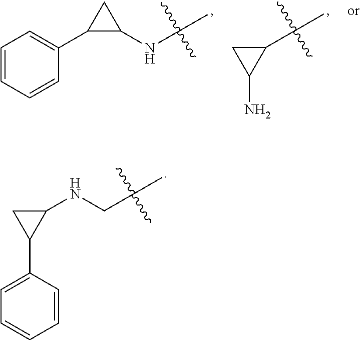 [SMILES: *NC1CC1c1ccccc1 ; *C1CC1N ; *CNC1CC1c1ccccc1]

In some embodiments, the compound has one, two, three, or more of the following features:

a) Q¹ is CR⁶;

b) R¹ is NH₂.

c) R² is F;

d) R⁴ is H;

e) R⁵ is CH₂CH₃;

f) R⁶ is H;

g) R⁷ is CO₂H;

e) R³ is

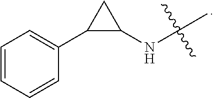 [SMILES: *NC1CC1c1ccccc1]

In some embodiments, the compound is of Formula (Ia), (Ib), (Ic), (Id), (Ie), (If), (Ig), (Ih), (Ii), (Ij), or (Ik):

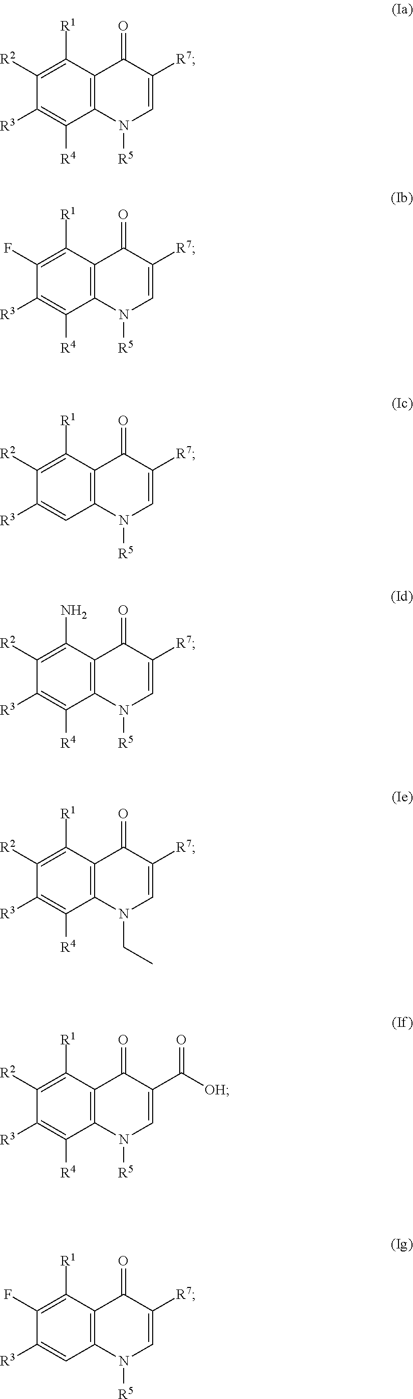

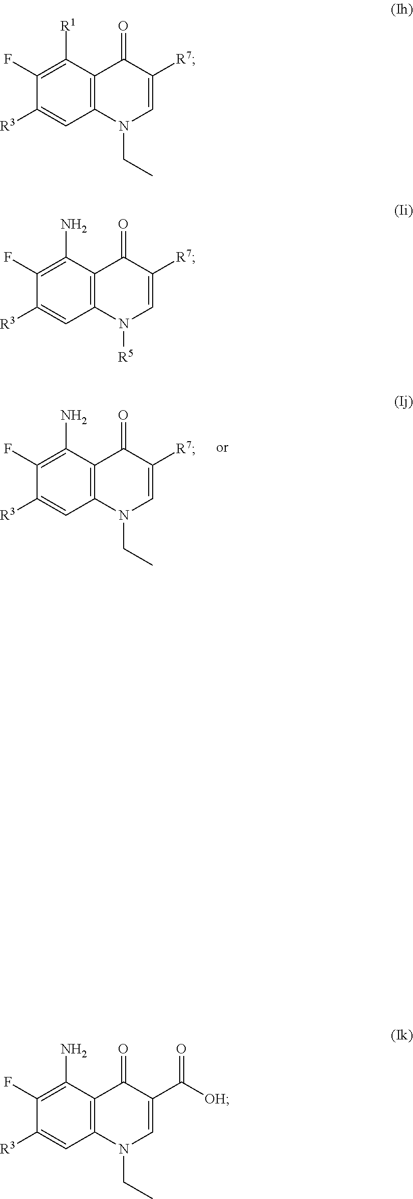

or a pharmaceutically acceptable salt or tautomer thereof, wherein R¹, R², R³, R⁴, R⁵, and R⁷ are as described herein.

In some embodiments, the compound is of Formula (II):

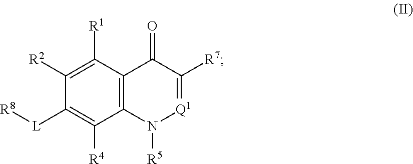

or a pharmaceutically acceptable salt or tautomer thereof, wherein Q¹, R¹, R², R⁴, R⁵, R⁷, R⁸, and L are as described herein.

In some embodiments, the compound is of Formula (IIIa), (IIIb), (IIIc), or (IIId):

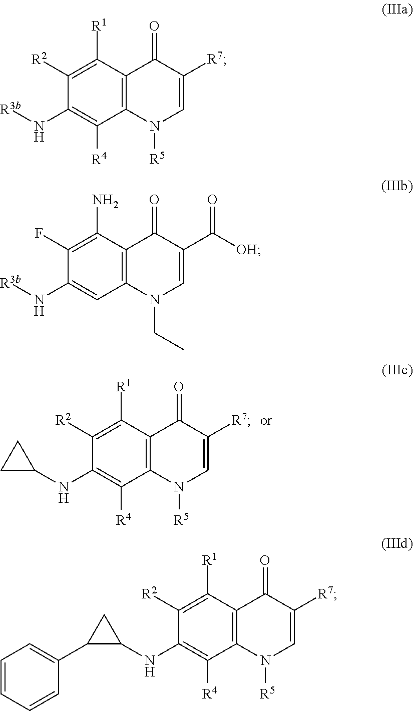

or a pharmaceutically acceptable salt or tautomer thereof, wherein R¹, R², R^{3b}, R⁴, R⁵ and R⁷ are as described herein. In some embodiments, the compound is of Formula (Iva), (Ivb), (Ivc), or (Ivd):

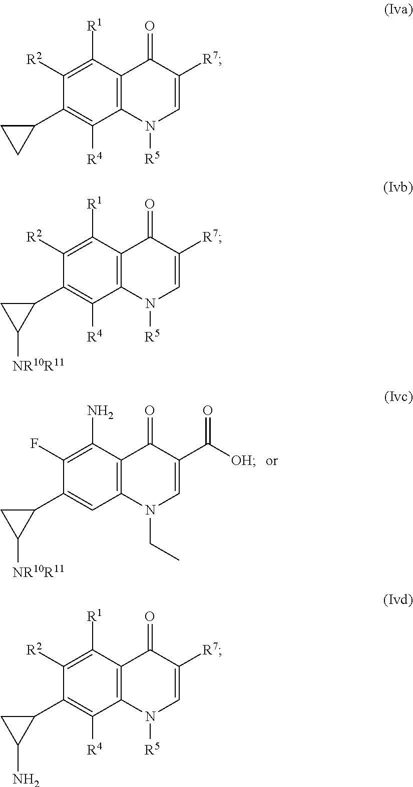

or a pharmaceutically acceptable salt or tautomer thereof, wherein R¹, R², R⁴, R⁵, R⁷, R¹⁰ and R¹¹ are as described herein.

In some embodiments, the compound is of Formula (Va), (Vb), (Vc), or (Vd):

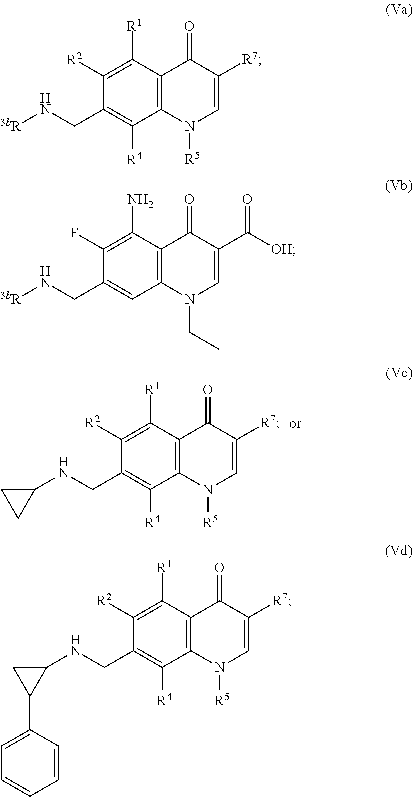

or a pharmaceutically acceptable salt or tautomer thereof, wherein R¹, R², R^{3b}, R⁴, R⁵ and R⁷ are as described herein.

In some embodiments, the compound is of Formula (Via), (Vib), (Vic), (Vid), (Vie), (Vif), (Vig), (Vih), (Vii), (Vij), or (Vik):

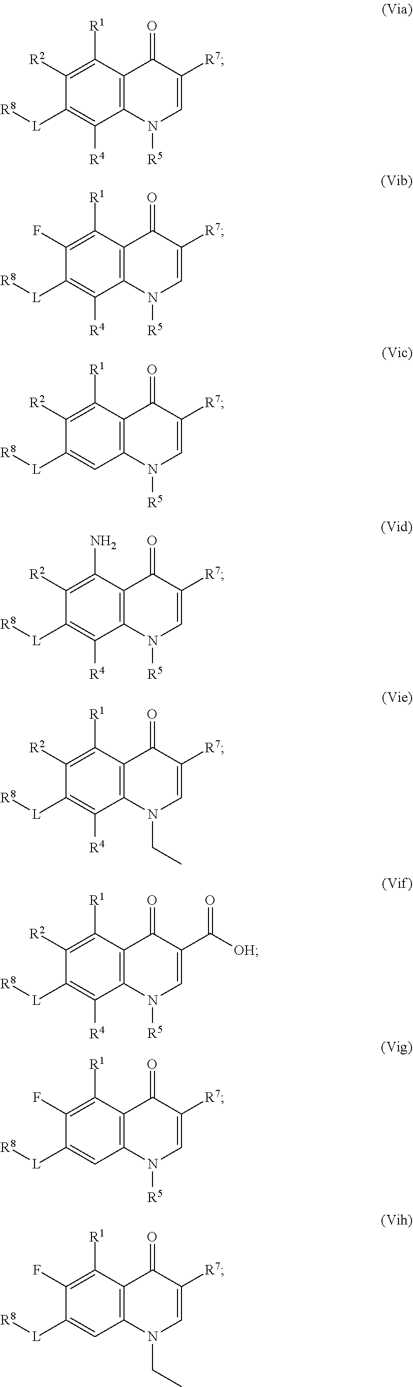

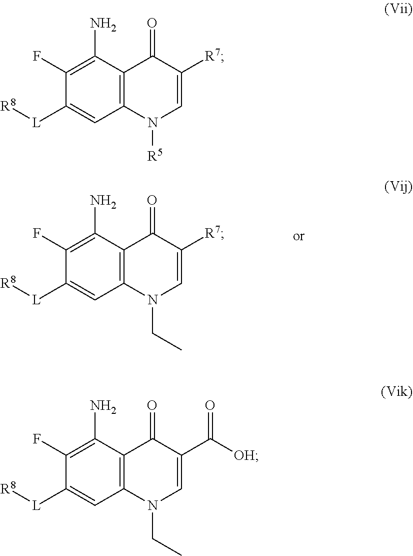

or a pharmaceutically acceptable salt or tautomer thereof, wherein R¹, R², R⁴, R⁵, R⁷, L and R⁸ are as described herein.

In some embodiments, the compound is of (VIIa) or (VIIb):

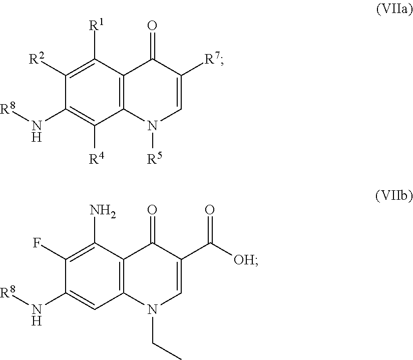

or a pharmaceutically acceptable salt or tautomer thereof, wherein R¹, R², R⁴, R⁵, R⁷ and R⁸ are as described herein.

In some embodiments, the compound is of Formula (VIIIa) or (VIIIb):

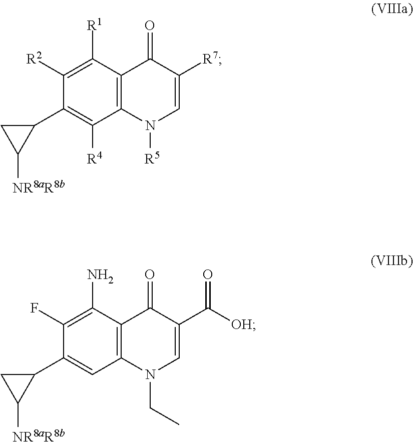

or a pharmaceutically acceptable salt or tautomer thereof, wherein R¹, R², R⁴, R⁵, R⁷, R^{8a} and R^{8b} are as described herein.

In some embodiments, the compound is of Formula (Ixa) or (Ixb):

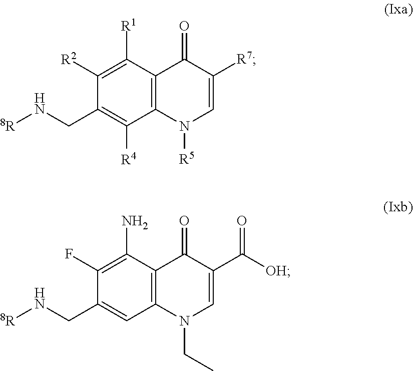

or a pharmaceutically acceptable salt or tautomer thereof, wherein R¹, R², R⁴, R⁵, R⁷ and R⁸ are as described herein.

In some embodiments, the compounds of the present disclosure show inhibitory activity against GSK3alpha and GSK3beta. In some embodiments, the compounds of the present disclosure have a measured IC₅₀ value of about 0.1 nM to about 10 μM, about 0.5 nM to about 5 μM, about 1 nM to about 1 μM, about 2 nM to about 900 nM, about 3 nM to about 800 nM, about 4 nM to about 700 nM, about 5 nM to about 600 nM, about 10 nM to about 500 nM, about 20 nM to about 400 nM, about 30 nM to about 300 nM, about 40 nM to about 250 nM, about 50 nM to about 200 nM, about 60 nM to about 150 nM, about 70 nM to about 100 nM, or about 80 nM to about 90 nM against GSK3alpha and/or GSK3beta.

In some embodiments, the compounds of the present disclosure show inhibitory activity against GSK3alpha and GSK3beta. In some embodiments, the compounds of the present disclosure have a measured IC₅₀ value of about 0.1 nM or less, about 1 nM or less, about 2 nM or less, about 3 nM or less, about 4 nM or less, about 5 nM or less, about 10 nM or less, about 25 nM or less, about 50 nM or less, about 100 nM or less, about 250 nM or less, about 500 nM or less, about 1 μM or less, or about 10 μM or less against GSK3alpha and GSK3beta.

In some embodiments, the compounds of the present disclosure show inhibitory activity against LSD-1 or LSD-2. In some embodiments, the compounds of the present disclosure have a measured inhibitory activity value of about 0% to about 100%, about 1% to about 95%, about 2% to about 90%, about 3% to about 85%, about 4% to about 80%, about 5% to about 75%, about 10% to about 70%, about 15% to about 65%, about 20% to about 60%, about 25% to about 55%, about 30% to about 50%, about 35% to about 45%, or about 40% to about 45% against LSD-1 or LSD-2.

In some embodiments, the compounds of the present disclosure show inhibitory activity against LSD-1 or LSD-2. In some embodiments, the compounds of the present disclosure have a measured inhibitory activity value of about 1% or greater, about 2% or greater, about 3% or greater, about 4% or greater, about 5% or greater, about 10% or greater, about 15% or greater, about 20% or greater, about 25% or greater, about 30% or greater, about 40% or greater, about 50% or greater, about 60% or greater, about 70% or greater, about 80% or greater, about 90% or greater, or about 95% or greater against LSD-1 or LSD-2.

In some embodiments, the compounds of the present disclosure show inhibitory activity against Foxo-1. In some embodiments, the compounds of the present disclosure have a measured IC₅₀ value of about 0.1 nM to about 10 μM, about 0.5 nM to about 5 μM, about 1 nM to about 1 μM, about 2 nM to about 900 nM, about 3 nM to about 800 nM, about 4 nM to about 700 nM, about 5 nM to about 600 nM, about 10 nM to about 500 nM, about 20 nM to about 400 nM, about 30 nM to about 300 nM, about 40 nM to about 250 nM, about 50 nM to about 200 nM, about 60 nM to about 150 nM, about 70 nM to about 100 nM, or about 80 nM to about 90 nM against Foxo-1.

In some embodiments, the compounds of the present disclosure show inhibitory activity against Foxo-1. In some embodiments, the compounds of the present disclosure have a measured IC₅₀ value of about 0.1 nM or less, about 1 nM or less, about 2 nM or less, about 3 nM or less, about 4 nM or less, about 5 nM or less, about 10 nM or less, about 25 nM or less, about 50 nM or less, about 100 nM or less, about 250 nM or less, about 500 nM or less, about 1 μM or less, or about 10 μM or less against Foxo-1.

In some embodiments, the compounds of the present disclosure show effective concentration of Lgr5+ expansion. In some embodiments, the compounds of the present disclosure have a measured EC value of about 0.1 nM to about 100 μM, about 0.5 nM to about 10 μM, about 1 nM to about 1 μM, about 2 nM to about 900 nM, about 3 nM to about 800 nM, about 4 nM to about 700 nM, about 5 nM to about 600 nM, about 10 nM to about 500 nM, about 20 nM to about 400 nM, about 30 nM to about 300 nM, about 40 nM to about 250 nM, about 50 TIM to about 200 nM, about 60 nM to about 150 nM, about 70 nM to about 100 nM, or about 80 nM to about 90 nM for Lgr5+ expansion.

In some embodiments, the compounds of the present disclosure show effective concentration of Lgr5+ expansion. In some embodiments, the compounds of the present disclosure have a measured EC value of about 0.1 nM or less, about 1 nM or less, about 2 nM or less, about 3 nM or less, about 4 nM or less, about 5 nM or less, about 10 nM or less, about 25 nM or less, about 50 nM or less, about 100 nM or less, about 250 nM or less, about 500 nM or less, about 1 μM or less, about 10 μM, or about 100 μM or less for Lgr5+ expansion.

In some embodiments, the compounds of the present disclosure show an increase in the percentage of Lgr5+ cells. In some embodiments, the compounds of the present disclosure increase the percentage of Lgr5+ to about 0% to about 100%, about 1% to about 95%, about 2% to about 90%, about 3% to about 85%, about 4% to about 80%, about 5% to about 75%, about 10% to about 70%, about 15% to about 65%, about 20% to about 60%, about 25% to about 55%, about 30% to about 50%, about 35% to about 45%, or about 40% to about 45%

In some embodiments, the compounds of the present disclosure show an increase in the percentage of Lgr5+ cells. In some embodiments, the compounds of the present disclosure increase the percentage of Lgr5+ cells to about 1% or greater, about 2% or greater, about 3% or greater, about 4% or greater, about 5% or greater, about 10% or greater, about 15% or greater, about 20% or greater, about 25% or greater, about 30% or greater, about 40% or greater, about 50% or greater, about 60% or greater, about 70% or greater, about 80% or greater, about 90% or greater, or about 95% or greater

In some embodiments, the compounds of the present disclosure show apparent permeability in basolateral to apical direction Papp (B-A) in Caco-2 cells. In some embodiments, the compounds of the present disclosure have a measured Papp (B-A) coefficient of about 0.1 to about 50, about 0.25 to about 40, about 0.5 to about 35, about 0.75 to about 30, about 1 to about 25, about 2 to about 20, about 3 to about 15, about 4 to about 10, about 5 to about 9, about 6 to about 8, or about 6 to about 7.

In some embodiments, the compounds of the present disclosure show apparent permeability in basolateral to apical direction Papp (B-A) in Caco-2 cells. In some embodiments, the compounds of the present disclosure have a measured Papp (B-A) coefficient of about 0.1 or greater, about 0.25 or greater, about 0.5 or greater, about 0.75 or greater, about 1 or greater, about 2 or greater, about 3 or greater, about 4 or greater, about 5 or greater, about 10 or greater, about 15 or greater, about 20 or greater, about 25 or greater, about 30 or greater, about 35 or greater, about 40 or greater, about 45 or greater, or about 50 or greater.

In some embodiments, the compounds of the present disclosure show efflux ratios in Caco-2 cells. In some embodiments, the compounds of the present disclosure have a measured efflux ratio of about 0.1 to about 50, about 0.25 to about 40, about 0.5 to about 35, about 0.75 to about 30, about 1 to about 25, about 2 to about 20, about 3 to about 15, about 4 to about 10, about 5 to about 9, about 6 to about 8, or about 6 to about 7.

In some embodiments, the compounds of the present disclosure show efflux ratios in Caco-2 cells. In some embodiments, the compounds of the present disclosure have a measured efflux ratio of about 0.1 or greater, about 0.25 or greater, about 0.5 or greater, about 0.75 or greater, about 1 or greater, about 2 or greater, about 3 or greater, about 4 or greater, about 5 or greater, about 10 or greater, about 15 or greater, about 20 or greater, about 25 or greater, about 30 or greater, about 35 or greater, about 40 or greater, about 45 or greater, or about 50 or greater.

In some embodiments, the compounds of the present disclosure show solubility in H₂O at pH 7.4. In some embodiments, the compounds of the present disclosure show a measured concentration of about 0.001 μM to about 100 μM, about 0.002 μM to about 90 μM, about 0.005 μM to about 80 μM, about 0.01 μM to about 70 μM, about 0.05 μM to about 60 μM, about 0.1 μM to about 50 μM, about 0.5 μM to about 40 μM, about 1 μM to about 30 μM, about 2 μM to about 25 μM, about 3 μM to about 20 μM, about 4 μM to about 15 μM, about 5 μM to about 10 μM, about 6 μM to about 9 μM, or about 7 μM to about 8 μM in H₂O at pH 7.4.

In some embodiments, the compounds of the present disclosure show solubility in H₂O at pH 7.4. In some embodiments, the compounds of the present disclosure show measured concentrations of about 0.001 μM or greater, about 0.002 μM or greater, about 0.005 μM or greater, about 0.01 μM or greater, about 0.05 μM or greater, about 0.1 μM or greater, about 0.5 μM or greater, about 1 μM or greater, about 2 μM or greater, about 3 μM or greater, 4 μM or greater, about 5 μM or greater, about 10 μM or greater, about 25 μM or greater, about 50 μM or greater, or about 100 μM or greater in H₂O at pH 7.4.

In some embodiments, the administration of the compound to a subject results into a higher Lgr5+ cell number in the subject, as compared to a comparable subject not being administered with the compound, by a factor ranging from about 2 fold to about 2,000,000 fold, from about 10 fold to about 1,000,000 fold, from about 100 fold to about 100,000 fold, or from about 1,000 fold to about 10,000 fold.

In some embodiments, the administration of the compound to a subject results into a higher Lgr5+ cell number in the subject, as compared to a comparable subject not being administered with the compound, by a factor of greater than about 10 fold, greater than about 10,000 fold, greater than about 100,000 fold, or greater than about 1,000,000 fold.

In some embodiments, the administration of the compound to a subject results into a higher Lgr5+ cell number in the subject, as compared to a comparable subject being administered with a Wnt agonist, by a factor ranging from about 0.1 fold to about 10 fold, from about 0.5 fold to about 5 fold, from about 1 fold to about 4 fold, or from about 1.5 fold to about 3 fold.

In some embodiments, the administration of the compound to a subject results into a higher Lgr5+ cell number in the subject, as compared to a comparable subject being administered with a Wnt agonist, by a factor of greater than about 1.5 fold, greater than about 2 fold, greater than about 2.5 fold, greater than about 3 fold, greater than about 3.5 fold, or greater than about 4 fold.

In some embodiments, the administration of the compound in combination with sodium valproate to a subject results into a higher percentage of Lgr5+ cells in the subject, as compared to a comparable subject being administered with the compound without sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

In some embodiments, the administration of the compound in combination with sodium valproate to a subject results into a higher percentage of Lgr5+ cells in the subject, as compared to a comparable subject being administered with the compound without sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

In some embodiments, the administration of the compound in combination with an LSD-1 inhibitor to a subject results into a higher percentage of Lgr5+ cells in the subject, as compared to a comparable subject being administered with the compound without the LSD-1 inhibitor, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

In some embodiments, the administration of the compound in combination with an LSD-1 inhibitor to a subject results into a higher percentage of Lgr5+ cells in the subject, as compared to a comparable subject being administered with the compound without the LSD-1 inhibitor, by a factor of greater than about 1 fold, greater than about 1.5 fold, greater than about 2 fold, greater than about 2.5 fold, greater than about 3 fold, greater than about 3.5 fold, or greater than about 4 fold.

In some embodiments, the administration of the compound in combination with sodium valproate and an LSD-1 inhibitor to a subject increases the percentage of Lgr5+ cells in the subject, as compared to a comparable subject being administered with the compound and sodium valproate without the LSD-1 inhibitor, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

In some embodiments, the administration of the compound in combination with sodium valproate and an LSD-1 inhibitor to a subject increases the percentage of Lgr5+ cells in the subject, as compared to a comparable subject being administered with the compound and sodium valproate without the LSD-1 inhibitor, by a factor of greater than about 1 fold, greater than about 1.5 fold, greater than about 2 fold, greater than about 2.5 fold, greater than about 3 fold, greater than about 3.5 fold, or greater than about 4 fold.

In some embodiments, the administration of the compound in combination with sodium valproate and an LSD-1 inhibitor to a subject increases the percentage of Lgr5+ cells in the subject, as compared to a comparable subject being administered with the compound and the LSD-1 inhibitor without sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

In some embodiments, the administration of the compound in combination with sodium valproate and an LSD-1 inhibitor to a subject increases the percentage of Lgr5+ cells in the subject, as compared to a comparable subject being administered with the compound and the LSD-1 inhibitor without sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

In some embodiments, the administration of the compound in combination with sodium valproate and an LSD-1 inhibitor to a subject increases the percentage of Lgr5+ cells in the subject, as compared to a comparable subject being administered with the compound without the LSD-1 inhibitor or sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

In some embodiments, the administration of the compound in combination with sodium valproate and an LSD-1 inhibitor to a subject increases the percentage of Lgr5+ cells in the subject, as compared to a comparable subject being administered with the compound without the LSD-1 inhibitor or sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

Unless otherwise stated, structures depicted herein are also meant to include compounds which differ only in the presence of one or more isotopically enriched atoms. For example, compounds having the present structure except for the replacement of a hydrogen atom by deuterium or tritium, or the replacement of a carbon atom by ¹³C or ¹⁴C, or the replacement of a nitrogen atom by ¹⁵N, or the replacement of an oxygen atom with ¹⁷O or ¹⁸O are within the scope of the present disclosure. Such isotopically labeled compounds are useful as research or diagnostic tools. In some embodiments, deuteration can be used to slow metabolism and thus potentially improve the compound half-life. Any or all hydrogens in the compound can be replaced with deuterium.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described herein and prodrugs and pharmaceutically acceptable salts thereof.

In some embodiments, the compound is an isotopic derivative of any one of the compounds described herein and pharmaceutically acceptable salts thereof.

In some embodiments, the compound is an isotopic derivative of any one of the compounds described herein.

It is understood that the isotopic derivative can be prepared using any of a variety of art-recognised techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

In some embodiments, the compound is a deuterium labeled compound of any one of the compounds described herein and prodrugs and pharmaceutically acceptable salts thereof.

In some embodiments, the compound is a deuterium labeled compound of any one of the compounds described herein and pharmaceutically acceptable salts thereof.

In some embodiments, the compound is a deuterium labeled compound of any one of the compounds described herein.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognised techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the invention or a pharmaceutically acceptable salt or solvate thereof that contains the aforementioned deuterium atom(s) is within the scope of the invention. Further, substitution with deuterium (i.e., 2H) may afford certain therapeutic advantages resulting from greater metabolic stability, e.g., increased in vivo half-life or reduced dosage requirements.

Also disclosed are alternative compounds which differ from the compounds disclosed herein in that one or more quaternary carbon atoms have been replaced by one or more silicon atoms, wherein the groups that were bonded to the carbon atom are bonded to the silicon atom. As a non-limiting example, a —C(CH₃)₃ moiety can be replaced by a —Si(CH₃)₃ moiety. In some embodiments, derivatives of the disclosed compounds are provided where one or more quaternary carbon atoms are replaced with one or more silicon atoms.

In some embodiments, a pharmaceutically acceptable salt or tautomer of a compound of the present disclosure, where one or more quaternary carbon atoms have been replaced by one or more silicon atoms, are within the disclosure. In some embodiments, a pharmaceutically acceptable salt or tautomer of a compound of the present disclosure, where one or more quaternary carbon atoms have been replaced by one or more silicon atoms, can be present in the pharmaceutical composition of the present disclosure.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1000 daltons. More usually, the molecular weight of the compound will be less than 900, for example less than 800, or less than 750, or less than 700, or less than 650 daltons. More conveniently, the molecular weight is less than 600 and, for example, is 550 daltons or less.

A suitable pharmaceutically acceptable salt of a compound of the disclosure is, for example, an acid-addition salt of a compound of the disclosure which is sufficiently basic, for example, an acid-addition salt with, for example, an inorganic or organic acid, for example hydrochloric, hydrobromic, sulfuric, phosphoric, trifluoroacetic, formic, citric methane sulphonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammolnium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Calm et al., Experientia 1956, 12, 81; Calm, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Calm and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual I- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”. 4^{th} edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulphate, bisulphate, sulphamate, nitrate, phosphate, citrate, methanesulphonate, trifluoroacetate, glutamate, glucuronate, glutarate, malate, maleate, succinate, fumarate, tartrate, tosylate, salicylate, lactate, naphthalenesulphonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammolnium cation such as tetramethylammolnium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulphonamides, tetrazoles, sulphonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

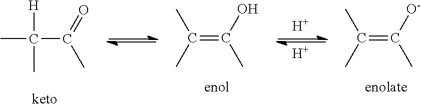

Compounds of any one of the Formulae disclosed herein containing an amine function may also form N-oxides. A reference herein to a compound of Formula (I) that contains an amine function also includes the N-oxide. Where a compound contains several amine functions, one or more than one nitrogen atom may be 102 ensorin to form an N-oxide. Particular examples of N-oxides are the N-oxides of a tertiary amine or a nitrogen atom of a nitrogen-containing heterocycle. N-oxides can be formed by treatment of the corresponding amine with an 102 ensorine agent such as hydrogen peroxide or a peracid (e.g., a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable akyl or acyl substitutents at the sulphonylurea group in a compound of the any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammolnia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesizing the Disclosed Compounds

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions 105 ensorin.

The compounds of the present disclosure may be made by a variety of methods, including standard chemistry. Suitable synthetic routes are depicted in the schemes given below.

The compounds of any of the formulae described herein may be prepared by methods known in the art of organic synthesis as set forth in part by the following synthetic schemes and examples. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of the present disclosure.

The compounds described herein may be made from commercially available starting materials or synthesized using known organic, inorganic, and/or enzymatic processes.

The compounds of the present disclosure can be prepared in a number of ways well known to those skilled in the art of organic synthesis. By way of example, compounds of the disclosure can be synthesized using the methods described below, together with synthetic methods known in the art of synthetic organic chemistry, or variations thereon as appreciated by those skilled in the art. These methods include but are not limited to those methods described below.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammolnia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of the formulae of the present disclosure has been 107 ensorineur by any one of the processes defined herein, the processes may then further comprise the additional steps of: (i) removing any protecting groups present; (ii) converting the compound formulae of the present disclosure into another compound of formulae of the present disclosure; (iii) forming a pharmaceutically acceptable salt, hydrate or solvate thereof; and/or (iv) forming a prodrug thereof.

The resultant compounds of formulae of the present disclosure can be isolated and purified using techniques well known in the art.

Conveniently, the reaction of the compounds is carried out in the presence of a suitable solvent. In some embodiments, the solvent is inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane (DCM); alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulphoxides, such as dimethyl sulphoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by 108 ensorine the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily 108 ensorine which kind of reagents and reactions conditions are to be used and how they are to be applied and adapted in any particular instance—wherever necessary or useful—in order to obtain the compounds of the present disclosure. Furthermore, some of the compounds of the present disclosure can readily be 109 ensorineur by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M Wuts, T. W. Greene, “Greene's Protective Groups in Organic Synthesis”, 4^{th} edition (2006) (John Wiley & Sons).

Those skilled in the art will recognize if a stereocenter exists in any of the compounds of the present disclosure. Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

It is understood that a neutral compound of Formula (I) may be converted to a salt (e.g., sodium salt) using routine techniques in the art (e.g., pH adjustment and, optionally, extraction (e.g., into an organic phase)). Further, a salt (e.g., sodium salt) of a compound of Formula (I) may be converted to a neutral compound using routine techniques in the art (e.g., pH adjustment and, optionally, extraction (e.g., into an aqueous phase)).

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected described herein.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammolnio)propyl-β-cyclodextrin, glucosyl-β-cyclodextrin, sulphated β-cyclodextrin (S-β-CD), maltosyl-β-cyclodextrin, β-cyclodextrin sulphobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammolnium salts such as benzalkonium halides, chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof. In some embodiments, the preservative is benzalkonium chloride.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH, the formulation may contain a pH modifying agent. In some embodiments, the pH range is about 5.0 to about 9.0, about 5.5 to about 8.5, about 6.0 to about 8.5, about 7.0 to about 8.5, about 7.2 to about 7.7, about 7.1 to about 7.9, or about 7.5 to about 8.0. In some embodiments, the pH is about 5.5 to about 8.5. In some embodiments, the pH is about 6.0 to about 8.5. The pH modifying agent is typically a mineral acid or metal hydroxide base, selected from the group of potassium hydroxide, sodium hydroxide, and hydrochloric acid, and mixtures thereof, and sodium hydroxide and or hydrochloric acid. In some embodiments, the pH modifying agent is sodium hydroxide. In some embodiments, the pH modifying agent is hydrochloric acid. In some embodiments, the pH modifying agent is sodium hydroxide and hydrogen chloride. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to 112 ensorine the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavouring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, known in the art. Thus, compositions intended for oral use may contain, for example, one or more colouring, sweetening, flavouring and/or preservative agents.

An effective amount of a compound of the present disclosure for use in therapy is an amount sufficient to treat or prevent an inflammasome related condition referred to herein, slow its progression and/or reduce the symptoms associated with the condition.

The size of the dose for therapeutic or prophylactic purposes of a compound of Formula (I) may vary according to the nature and severity of the conditions, the age and sex of the animal or patient, and the route of administration, according to well-known principles of medicine.

FOXO-1, GSK3 α/β and/or LSD-1 Inhibitors

Lysine specific demethylase 1 (LSD-1; also known as KDM1A, AOF2, BHC110 or KIAA0601) is a histone H3K4me1/2 demethylase found in various transcriptional co-repressor complexes. These complexes include Histone Deacetylases (HDAC1/2) and Co-Repressor for Element-1-Silencing Transcription factor (CoREST). LSD-1 mediated H3K4 demethylation can result in a repressive chromatin environment that silences gene expression. LSD-1 has been shown to play a role in development in various contexts. LSD-1 can interact with pluripotency factors in human embryonic stem cells and is important for decommissioning enhancers in stem cell differentiation. Beyond embryonic settings, LSD-1 is also critical for hematopoietic differentiation. LSD-1 is overexpressed in multiple cancer types and recent studies suggest inhibition of LSD-1 reactivates the all-trans retinoic acid receptor pathway in acute myeloid leukemia (AML). These studies implicate LSD-1 as a key regulator of the epigenome that modulates gene expression through post-translational modification of histones and through its presence in transcriptional complexes.

Forkhead box protein O1 (FOXO1) also known as forkhead in rhabdomyosarcoma is a protein that in humans is encoded by the FOXO1 gene. FOXO1 is a transcription factor that plays important roles in regulation

of gluconeogenesis and glycogenolysis by insulin signaling, and is also central to the decision for a preadipocyte to commit to adipogenesis. It is primarily regulated through phosphorylation on multiple residues; its transcriptional activity is dependent on its phosphorylation state.

Glycogen synthase kinase 3 (GSK3) is a serine/threonine protein kinase that mediates the addition of phosphate molecules onto serine and threonine amino acid residues. First discovered in 1980 as a regulatory kinase for its namesake, Glycogen synthase, GSK3 has since been identified as a kinase for over forty different proteins in a variety of different pathways. In mammals GSK3 is encoded by two known genes, GSK3 alpha (GSK3α) and GSK3 beta (GSK3β). GSK3 has recently been the subject of much research because it has been implicated in a number of diseases, including Type II diabetes (Diabetes mellitus type 2), Alzheimer's Disease, inflammation, cancer, and bipolar disorder.

Thus, a “FOXO-1, GSK3 α/β and/or LSD-1 inhibitor” refers to an compound capable of the decreasing the expression or enzymatic activity of FOXO-1, GSK3 α/β and/or LSD-1. For example a FOXO-1, GSK3 α/β and/or LSD-1 inhibitor results in a change in methylation and/or phosphorylation of a target gene in a cell, for instance, in a cochlear cell.

In some embodiments, a FOXO-1, GSK3 α/β and/or LSD-1 inhibitor decreases the expression or enzymatic activity of FOXO-1, GSK3 α/β and/or LSD-1 by at least 5, 10, 20, 30, 40, 50, 60, 70, 80, 90, or 100% relative to a control, for example relative to a baseline level of activity.

In some embodiments, a FOXO-1, GSK3 α/β and/or LSD-1 inhibitor changes methylation and/or phosphorylation of a target gene by at least 5, 10, 20, 30, 40, 50, 60, 70, 80, 90, or 100% relative to a control, for example relative to a baseline level of activity.

In some instances, a FOXO-1, GSK3 α/β and/or LSD-1 inhibitor increases expression or activity of a target gene by at least 5, 10, 20, 30, 40, 50, 60, 70, 80, 90, or 100% relative to a control, for example relative to a baseline level of activity.

In some instances, a FOXO-1, GSK3 α/β and/or LSD-1 inhibitor changes methylation and/or phosphorylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some instances, a FOXO-1, GSK3 α/β and/or LSD-1 inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In particular embodiments, an agent having activity as a FOXO-1, GSK3 α/β and/or LSD-1 inhibitor has an IC50 in an in vitro FOXO-1, GSK3 α/β and/or LSD-1 functional assay ranging from about 10 nM to 100 μM, about 0.1 nM to 1 nM, about 1 nM to 10 nM, about 10 nM to 100 nM, about 100 nM to 1 μM, about 1 μM to 10 μM, about 10 μM to about 100 μM, or about 100 μM to 1000 μM.

In some instances a FOXO-1, GSK3 α/β and/or LSD-1 inhibitor is reversible. In other instances the FOXO-1, GSK3 α/β and/or LSD-1 inhibitor is irreversible.

In some embodiments, the compounds of the present disclosure are LSD-1 inhibitors.

In some embodiments, the compounds of the present disclosure are FOXO-1 inhibitors.

In some embodiments, the compounds of the present disclosure are GSK3 α/β inhibitors.

In some embodiments, the compounds of the present disclosure are FOXO-1 and GSK3 α/β inhibitors.

In some embodiments, the compounds of the present disclosure are FOXO-1 and LSD-1 inhibitors.

In some embodiments, the compounds of the present disclosure are LSD-1 and GSK3 α/β inhibitors.

In some embodiments, the compounds of the present disclosure are FOXO-1, GSK3 α/β and LSD-1 inhibitors.

### Methods of Using the Disclosed Compounds

The present disclosure relates to methods to activate a cell cycle progression pathway (e.g., the Wnt pathway) and/or inhibiting one or more of FOXO-1, GSK3 α/β and LSD-1 activity. Although there are several purported inhibitors in the patent and non patent literature, not all FOXO-1, GSK3 α/β and LSD-1 inhibitors when administered in the absence of other therapeutic agents would be sufficient nor potent enough to promote the desired level of activation of stem cell proliferation. In addition, the inhibitors of the present disclosure may be effective in inhibiting at least two of FOXO-1, GSK3 α/β and LSD-1 with the single agent. In addition, the compounds and/or inhibitors of the present disclosure may be effective inhibitors in a manner that eliminates or reduces the need for using multiple agents.

In another aspect the present disclosure relates to methods to prevent, reduce or treat the incidence and/or severity of disorders or diseases associated with absence or lack of certain tissue cells. In one aspect the present disclosure relates to methods to prevent, reduce or treat the incidence and/or severity of inner ear disorders and hearing impairments involving inner ear tissue, particularly inner ear hair cells, their progenitors, and optionally, the stria vascularis, and associated auditory nerves. Of particular interest are those conditions that lead to permanent hearing loss where reduced number of hair cells may be responsible and/or decreased hair cell function. Also of interest are those arising as an unwanted side-effect of ototoxic therapeutic drugs including cisplatin and its analogs, aminoglycoside antibiotics, salicylate and its analogs, or loop diuretics. In some embodiments, the present disclosure relates to inducing, promoting, or enhancing the growth, proliferation or regeneration of inner ear tissue, particularly inner ear supporting cells and hair cells.

Among other things, the methods presented here can be useful for the preparation of pharmaceutical formulations for the prophylaxis and/or treatment of acute and chronic ear disease and hearing loss, dizziness and balance problems especially of sudden hearing loss, acoustic trauma, hearing loss due to chronic noise exposure, presbycusis, trauma during implantation of the inner ear prosthesis (insertion trauma), dizziness due to diseases of the inner ear area, dizziness related and/or as a symptom of Meniere's disease, vertigo related and/or as a symptom of Meniere's disease, tinnitus, and hearing loss due to antibiotics and cytostatics and other drugs.

When cochlea supporting cell populations or vestibular supporting cell populations are treated with the compound, whether the population is in vivo or in vitro, the treated supporting cells exhibit stem-like behavior in that the treated supporting cells have the capacity to proliferate and differentiate and, more specifically, differentiate into cochlear hair cells. In some embodiments, the compound induces and maintains the supporting cells to produce daughter stem cells that can divide for many generations and maintain the ability to have a high proportion of the resulting cells differentiate into hair cells. In some embodiments, the proliferating stem cells express stem cell markers which may include, but are not limited to, Lgr5, Sox2, Opem1, Phex, lin28, Lgr6, cyclin D1, Msx1, Myb, Kit, Gdnf3, Zic3, Dppa3, Dppa4, Dppa5, Nanog, Esrrb, Rex1, Dnmt3a, Dnmt3b, Dnmt31, Utf1, Tcl1, Oct4, Klf4, Pax6, Six2, Zic1, Zic2, Otx2, Bmi1, CDX2, STAT3, Smad1, Smad2, smad2/3, smad4, smad5, and/or smad7.

In some embodiments, the method of the present disclosure may be used to maintain, or even transiently increase stemness (i.e., self-renewal) of a pre-existing supporting cell population prior to significant hair cell formation. In some embodiments, the pre-existing supporting cell population comprises inner pillar cells, outer pillar cells, inner phalangeal cells, Deiter cells, Hensen cells, Boettcher cells, and/or Claudius cells. Morphological analyses with immunostaining (including cell counts) and lineage tracing across a Representative Microscopy Samples may be used to confirm expansion of one or more of these cell-types. In some embodiments, the pre-existing supporting cells comprise Lgr5+ cells. Morphological analyses with immunostaining (including cell counts) and qPCR and RNA hybridization may be used to confirm Lgr5 upregulation amongst the cell population.

Advantageously, the methods of the present disclosure achieve these goals without the use of genetic manipulation. Germ-line manipulation used in many academic studies is not a therapeutically desirable approach to treating hearing loss. In some embodiments, the therapy involves the administration of a small molecule, peptide, antibody, or other non-nucleic acid molecule or nucleic acid delivery vector unaccompanied by gene therapy. In some embodiments, the therapy involves the administration of a small organic molecule. In some embodiments, hearing protection or restoration is achieved through the use of a (non-genetic) therapeutic that is injected in the middle ear and diffuses into the cochlea.

The cochlea relies heavily on all present cell types, and the organization of these cells is important to their function. As supporting cells play an important role in neurotransmitter cycling and cochlear mechanics. Thus, maintaining a rosette patterning within the organ of Corti may be important for function. Cochlear mechanics of the basilar membrane activate hair cell transduction. Due to the high sensitivity of cochlear mechanics, it is also desirable to avoid masses of cells. In all, maintaining proper distribution and relation of hair cells and supporting cells along the basilar membrane, even after proliferation, is likely a desired feature for hearing as supporting cell function and proper mechanics is necessary for normal hearing.

In some embodiments the hearing loss treated by using a composition as disclosed herein is 118 ensorineural hearing loss.

Sensorineural hearing loss accounts for approximately 90% of hearing loss and it often arises from damage or loss of hair cells in the cochlea. There are numerous causes of hair cell damage and loss, and the agents and treatments described herein may be used in the context of sensorineural hearing loss arising from any cause of hair cell damage or loss. For example, hair cells may be damage and loss may be induced by noise exposure, leading to noise-induced sensorineural hearing loss. Thus, in some embodiments sensorineural hearing loss is noise-induced sensorineural hearing loss. Noise-induced sensorineural hearing loss can be a result of chronic noise exposure or acute noise exposure. Ototoxic drugs, for example cisplatin and its analogs, aminoglycoside antibiotics, salicylate and its analogs, or loop diuretics, can also cause sensorineural hearing loss. In some embodiments sensorineural hearing loss is drug-induced sensorineural hearing loss. Infection may damage cochlear hair cells, and may be a cause of sudden sensorineural hearing loss. In some embodiments sensorineural hearing loss is sudden sensorineural hearing loss (SSNHL). Sudden sensorineural hearing can also be idiopathic. Hair cells can also be lost or damaged over time as part of the ageing process in humans. In some embodiments, sensorineural hearing loss is age-related sensorineural hearing loss (also known as presbycusis).

### Measurement Of Hearing Loss

Hearing loss can be assessed by several different tests. Such tests may determine the audibility of a sound to a patient and/or the intelligibility of the sound to a patient prior to or after treatment. The audibility of a sound is a measure of a patient's ability to detect the sound (i.e. whether the patient can determine the presence or absence of a sound). The intelligibility of a sound is a measure of a patient's ability to correctly identify the sound. For instance, hearing may be assessed according to whether a patient can correctly identify a word or not. A patient with hearing loss may therefore neither be able to detect a sound nor correctly identify it (i.e. the sound is inaudible and unintelligible). However, audibility is not necessarily associated with intelligibility, and a patient may, for example, be able detect a sound, but not correctly identify it (i.e. the sound is audible but unintelligible).

Pure Tone Audiometry

Assessment of a patient's audibility function is typically carried out by an audiologist using an audiometer in a hearing test known as pure tone audiometry. Pure tone audiometry is a standard test used to assess the audibility of a sounds and is described in detail elsewhere (see, for example, Katz, J., Medwetsky, L., Burkard, R., & Hood, L. (2009) Handbook of Clinical Audiology. Philadelphia, Pa.: Lippincott Williams and Wilkins). Pure tone audiometry is typically carried out in a sound-treated booth, which reduces ambient noise levels that may interfere with the detection of low-level sound stimuli.

In pure tone audiometry, a patient is exposed to pure tone stimuli at specific frequencies to determine the patient's hearing threshold at each frequency. Standard audiometry measures a patient's pure tone hearing threshold at each of the following frequencies 0.25 kHz, 0.5 kHz, 1 kHz, 2 kHz, 3 kHz, 4 kHz, 6 kHz and 8 kHz. However, a patient's hearing threshold does not need to be determined at all of these frequencies to ascertain whether or not the patient has sensorineural hearing loss. For instance, a subset frequencies, or a single frequency may be tested to identify a patient with sensorineural hearing loss.

To determine the hearing threshold, the volume of the pure tone is altered to determine the lowest level of stimuli that the patient is able to detect. The lowest level of stimuli (corresponding to the quietest sound) is the pure tone hearing threshold at a given frequency. The pure tone threshold is typically measured in a patient using according decibels in hearing level (dB HL) on an audiometer. However, hearing thresholds may also be determined using other methods known to the person skilled in the art. For example, hearing function may be measured by Auditory Brainstem Response (ABR) testing or Auditory Steady State Response (ASSR) testing. Other tests can also be used to determine hearing function in a patient. For instance, Distortion product optoacoustic emissions (DPOAEs) can be used to measure outer hair cell function and loss and may be used in differential diagnosis of hearing loss arising from hair cell loss from hearing loss associated with higher level processing (e.g., auditory neuropathy).

Pure tone thresholds may be plotted on a graph to produce an audiogram for the patient.

Pure tone thresholds measured across different frequencies may also be averaged to provide a pure tone average. For instance, a patient that has pure tone hearing thresholds of 50 dB HL at 0.5 Hz, 60 dB HL at 1 kHz, 65 dB HL at 2 kHz and 70 dB at 4 kHz would have a pure tone average of 61.25 dB HL, when measured across 0.5 kHz, 1 kHz, 2 kHz and 4 kHz.

Pure tone averages may be calculated across different frequencies. Pure tone thresholds at any subset of frequencies may be used to calculate pure tone averages. In some embodiments, the average of the patient hearing threshold is measured across 0.5 kHz, 1 kHz, 2 kHz and 4 kHz. In some embodiments, pure tone average is measured across 4 kHz, 6 kHz and 8 kHz. Measurement of pure tone average across 4 kHz, 6 kHz and 8 kHz is useful when seeking to assess the patient's hearing function at the higher frequencies within the standard audiometric frequencies.

Sensorineural hearing loss can be categorized according to its severity. The severity of hearing loss is determined by the hearing levels at which a threshold level is obtained in a patient by pure tone audiometry. Severity of hearing loss can be classified according to hearing thresholds using the following definitions:

Normal: 25 dB HL or less

Mild: at least 25 dB HL and no more than 40 dB HL

Moderate: at least 40 dB HL and no more than 55 dB HL

Moderately Severe: at least 55 dB HL and no more than 70 dB HL

Severe: at least 70 dB HL and no more than 90 dB HL

Profound: at least 90 dB HL or more

These measures of severity are standard measures in the field (see Goodman, A. (1965) Reference zero levels for pure tone audiometer. ASHA, 7, 262-263). In some embodiments, the severity of hearing loss is classified according to a patient's hearing threshold at a single frequency (for example, 0.25 kHz, 0.5 kHz, 1 kHz, 2 kHz, 3 kHz, 4 kHz, 6 kHz or 8 kHz). For example, a patient may have mild hearing loss at 8 kHz, and normal hearing at the other standard audiometric frequencies. In some embodiments, the severity of hearing loss is classified according to pure tone average, when measured across a subset of frequencies. In certain such embodiments, the severity of hearing loss is classified according to the pure tone average across 0.5 kHz, 1 kHz, 2 kHz and 4 kHz. For example, a patient may have moderate hearing loss according to their pure tone average across 0.5 kHz, 1 kHz, 2 kHz, and 4 kHz, but have moderately severe hearing loss at a single frequency (e.g., 8 kHz). In other embodiments, the severity of hearing loss is classified according to the pure tone average across 4 kHz, 6 kHz and 8 kHz.

A patient that has hearing threshold of 25 dB HL or less at standard audiometric frequencies (i.e., 0.25 kHz, 0.5 kHz, 1 kHz, 2 kHz, 3 kHz, 4 kHz, 6 kHz, and 8 kHz) has normal hearing. The patient's audiogram is also a normal audiogram.

### Word Recognition Tests

Alternatively, or in addition to pure tone audiometry, hearing loss may be assessed using a word recognition test. A word recognition test measures the patient's ability to correctly identify a word, thereby providing a measure of sound intelligibility (in particular, speech intelligibility) that may not be provided by pure tone audiometry. In some embodiments, a word recognition score is used to determine the patient's ability to correctly identify words prior to treatment.

A standard word recognition in quiet test, also referred to herein as a standard word recognition test, is a test administered by an audiologist that measures a patient's speech intelligibility in recognizing words in a quiet environment. A quiet environment is an environment with little to no background noise.

A standard word recognition test may be used to determine a person's ability to recognize words selected from a word list and presented to the patient at a given decibel (dB) level. In some embodiments, the standard word recognition test is used to determine a patient's ability to recognize words at more than one decibel level.

In some embodiments, the standard word recognition test assesses the patient's ability to identify 50 words. However, the number of words presented to the patient may be more or less than 50. For example, in some embodiments, the standard word recognition test is for 25 words. In other embodiments, the standard word recognition test is for 10 words.

A standard word recognition test may be used to generate a standard word recognition (%) score which is calculated using the formula:

$$standard word recognition score ( \% ) = 100 \times ( \frac{words recognised in standard word recognition test}{total words} )$$

In some embodiments, the standard word recognition score is expressed as the number of words that are correctly recognized in the test.

In some embodiments, a list of words is administered to each ear, and a standard word recognition score is calculated for each ear. Herein the results of the standard word recognition score refer to the ear that has been/will be treated.

A standard word recognition test may be carried out using any list of words.

However, standard word lists are typically used in a standard word recognition test. In some embodiments, each test word is embedded in a carrier phrase. Example of carrier phrases are: “Say the word ______ again”, “You will say ______”, or “Say the word ______”.

In some embodiments, the standard word recognition test is the Maryland consonant-vowel nucleus-consonant (CNC) word test. The Maryland CNC word test has been described, for example, in Mendel, L. L., Mustain, W. D., & Magro, J. (2014). Normative data for the Maryland CNC Test. Journal of the American Academy of Audiology, 25, 775-781.

The Maryland CNC word test is a standard word recognition test that uses phonemically balanced word lists comprising words that are consonant-nucleus-consonant (CNC) monosyllables. These CNC lists are balanced so that each initial consonant, each vowel, and each final consonant appears with the same frequency within each list. The Maryland CNC test has 10 lists of 50 words.

In some embodiments, the Maryland CNC Test uses words from Lehiste and Peterson's phonemically balanced word lists, all of which were CNC monosyllables, for example as described in Lehiste I, Peterson G E. (1959) Linguistic considerations in the study of speech intelligibility. Journal of the Acoustical Society of America 31(3): 280-286.

In some embodiments, the Maryland CNC Test uses words from revised CNC lists that eliminate rare literary words and proper names, for example as described in Peterson G E, Lehiste I. (1962) Revised CNC lists for auditory tests. Journal of Speech and Hearing Disorders 27:62-70.

In some embodiments, the Maryland CNC Test uses words from modified CNC word lists that take into consideration the effects of coarticulation, where the acoustic properties of phonemes are influenced by those phonemes that immediately precede and follow them, for example as described in Causey G D, Hood L J, Hermanson C L, Bowling L S. (1984) The Maryland CNC Test: normative studies. Audiology 23(6): 552-568. The words of the Maryland CNC test are spoken within the carrier phrase: ‘Say the ______ again,’

In some embodiments, the standard word recognition test is the C.I.D Auditory Test W-22 (CID W-22) test. The CID W-22 test has been described, for example, in Hirsh, I. J., Davis, H. Silverman, S. R., Reynolds, E. G., Eldert, E., & Benson, R. W. (1952). Development of Materials for Speech Audiometry. Journal of Speech, Language, and Hearing Research, 17(3), 321-337.

The CID W-22 test uses 200 monosyllabic words which are divided into four lists of 50 words each. Each list is phonetically balanced. The speech sounds within the list occur with the same relative frequency as they do in a representative sample of English speech. There are three criteria for the vocabulary in the phonetically balanced word lists. First, all the words must be one-syllable words with no repetition of words in the different lists. Second, any word chosen should be a familiar word. This second criterion is to minimize the effect of differences in the educational background of subjects. Third, the phonetic composition of each word list should correspond to that of English as a whole as closely as possible. The words of the CID W-22 test are spoken with the carrier phrase: “You will say ______”.

In some embodiments the standard word recognition test is the NU No. 6 test. The NU No. 6 has been described, for example, in Tillman, T. W., & Carhart, R (1966). An expanded test for speech discrimination utilizing CNC monosyllabic words: Northwestern University Auditory Test No. 6. Northwestern Univ Evanston Ill. Auditory Research Lab.

In some embodiments, the NU No. 6 test uses 4 lists of 50 words, for example, as described in Table 28-2 of Tillman, T. W., & Carhart, R. (1966). The words of the NU No. 6 test are spoken with the carrier phrase: “Say the word ______”.

In some embodiments the standard word recognition test is the Maryland CNC test, using the words list and carrier phrases as defined in Causey G D, Hood L J, Hermanson C L, Bowling L S. (1984) The Maryland CNC Test: normative studies. Audiology 23(6): 552-568. In certain such embodiments, the word signal is provided to the patient at 40 dB above speech perception level.

### Words-in-Noise (WIN) Test

A “Words-in-Noise (WIN) Test” is a test administered by an audiologist to measure a patient's speech intelligibility in recognizing words in the presence of background noise.

The WIN test consists of administering words to an ear at a varying signal-to-noise ratio (SNR) level. The signal-to-noise ratio is the ratio of the strength of the signal carrying information (e.g., the test word signal), relative to the signal of interference (e.g., noise), and is typically expressed in decibels. In some embodiments, the background noise is multi-talker babble at a fixed decibel level.

In some embodiments the multi-talker babble is comprised of six talkers (three female, three male) at a fixed level, for example, as described in Wilson, R. H., Abrams, H. B., & Pillion, A. L. (2003). A word-recognition task in multi-talker babble using a descending presentation mode from 24 dB to 0 dB signal to babble. Journal of Rehabilitation Research and Development, 40(4), 321-328.

In some embodiments, the background noise is maintained at a fixed decibel level, and the variation in the SNR decibel level is achieved by varying the decibel level of the test word signal. The SNR decibel level is therefore the SNR above the background noise. For example if the level of multi-talker babble is fixed at 70 dB SPL, and the level of the test word signal varied from 70 dB SPL to 94 dB SPL, this would give a SNR decibel level variation of 0 dB to 24 dB.

In some embodiments, the test words that are used may be from any list described herein for the word recognition tests. In some embodiments, the word-in-noise test is for 70 words. In other embodiments, the words-in-noise test is for 35 words.

In some embodiments, the test consists of administering 35 or 70 monosyllabic words from the NU No. 6 word lists. The test words may be spoken with the carrier phrase: “Say the word ______”.

In some embodiments, the WIN test is administered in a descending-level SNR paradigm. In these embodiments, the test words at the high SNR decibel level are presented first, followed by test words at gradually lower SNR decibel levels, with words at the lowest SNR decibel level administered last. The high SNR decibel level is the easiest setting for the patient to identify the signal words. The low SNR decibel levels is the most difficult setting for the patient to identify the signal words. In other embodiments, the WIN test is administered in a randomized-level SNR paradigm. In these embodiments, the test words are presented at different SNR decibel levels in a randomized order.

In some embodiments the SNR decibel level of the test words varies from 24 dB SNR (easiest condition) to 0 dB SNR (most difficult condition) in 4 dB decrements, for a total of seven SNR levels (i.e. 24 dB SNR, 20 dB SNR, 16 dB SNR 12 dB SNR, 8 dB SNR, 4 dB SNR and 0 dB SNR).

In some embodiments the WIN test consists of administering 70 monosyllabic words from the NU No. 6 word lists, where the SNR decibel level of the test words varies from 24 dB SNR (easiest condition) to 0 dB SNR (most difficult condition) in 4 dB decrements, for a total of seven SNR levels (i.e. 24 dB SNR, 20 dB SNR, 16 dB SNR, 12 dB SNR, 8 dB SNR, 4 dB SNR and 0 dB SNR). In this embodiment, the level of multi-talker babble is fixed at 70 dB SPL, and the level of the test word signal varies from 70 dB SPL to 94 dB SPL.

The ‘words-in-noise’ test may be used to generate a words-in-noise score.

In some embodiments the words-in-noise score is given as a percentage of the total correct words recognized by the patient in the test and calculated using the formula:

$$words n noise score ( \% ) = 100 \times ( \frac{words recognised in standard words in noise test}{total words} ) .$$

In some embodiments of the present disclosure, the cell density of hair cells in a cochlear cell population is expanded in a manner that maintains, or even establishes, the rosette pattern characteristic of cochlear epithelia.

In accordance with one aspect of the present disclosure, the cell density of hair cells may be increased in a population of cochlear cells comprising both hair cells and supporting cells. The cochlear cell population may be an in vivo population (i.e., comprised by the cochlear epithelium of a subject) or the cochlear cell population may be an in vitro (ex vivo) population. If the population is an in vitro population, the increase in cell density may be determined by reference to a Representative Microscopy Sample of the population taken prior and subsequent to any treatment. If the population is an in vivo population, the increase in cell density may be determined indirectly by determining an effect upon the hearing of the subject with an increase in hair cell density correlating to an improvement in hearing.

In some embodiments, supporting cells placed in a Stem Cell Proliferation Assay in the absence of neuronal cells form ribbon synapses.

In a native cochlea, patterning of hair cells and supporting cells occurs in a manner parallel to the basilar membrane. In some embodiments of the present disclosure, the proliferation of supporting cells in a cochlear cell population is expanded in a manner that the basilar membrane characteristic of cochlear epithelia.

In some embodiments, the number of supporting cells in an initial cochlear cell population is selectively expanded by treating the initial cochlear cell population with a compound or composition provided herein to form an intermediate cochlear cell population and wherein the ratio of supporting cells to hair cells in the intermediate cochlear cell population exceeds the ratio of supporting cells to hair cells in the initial cochlear cell population. The expanded cochlear cell population may be, for example, an in vivo population, an in vitro population or even an in vitro explant. In one such embodiment, the ratio of supporting cells to hair cells in the intermediate cochlear cell population exceeds the ratio of supporting cells to hair cells in the initial cochlear cell population. For example, in one such embodiment the ratio of supporting cells to hair cells in the intermediate cochlear cell population exceeds the ratio of supporting cells to hair cells in the initial cochlear cell population by a factor of 1.1. By way of further example, in one such embodiment the ratio of supporting cells to hair cells in the intermediate cochlear cell population exceeds the ratio of supporting cells to hair cells in the initial cochlear cell population by a factor of 1.5. By way of further example, in one such embodiment the ratio of supporting cells to hair cells in the intermediate cochlear cell population exceeds the ratio of supporting cells to hair cells in the initial cochlear cell population by a factor of 2. By way of further example, in one such embodiment the ratio of supporting cells to hair cells in the intermediate cochlear cell population exceeds the ratio of supporting cells to hair cells in the initial cochlear cell population by a factor of 3. In each of the foregoing embodiments, the capacity of a compound or composition of the present disclosure to expand a cochlear cell population as described in this paragraph may be determined by means of a Stem Cell Proliferation Assay.

In some embodiments, the number of stem cells in a cochlear cell population is expanded to form an intermediate cochlear cell population by treating a cochlear cell population with a compound or composition provided herein wherein the cell density of stem cells in the intermediate cochlear cell population exceeds the cell density of stem cells in the initial cochlear cell population. The treated cochlear cell population may be, for example, an in vivo population, an in vitro population or even an in vitro explant. In one such embodiment, the cell density of stem cells in the treated cochlear cell population exceeds the cell density of stem cells in the initial cochlear cell population by a factor of at least 1.1. For example, in one such embodiment the cell density of stem cells in the treated cochlear cell population exceeds the cell density of stem cells in the initial cochlear cell population by a factor of at least 1.25. For example, in one such embodiment the cell density of stem cells in the treated cochlear cell population exceeds the cell density of stem cells in the initial cochlear cell population by a factor of at least 1.5. By way of further example, in one such embodiment the cell density of stem cells in the treated cochlear cell population exceeds the cell density of stem cells in the initial cochlear cell population by a factor of at least 2. By way of further example, in one such embodiment the cell density of stem cells in the treated cochlear cell population exceeds the cell density of stem cells in the initial cochlear cell population by a factor of at least 3. In vitro cochlear cell populations may expand significantly more than in vivo populations; for example, in some embodiments the cell density of stem cells in an expanded in vitro population of stem cells may be at least 4, 5, 6, 7, 8, 9, 10, 15, 20, 25, 30, 35, 40, 45, 50, 75, 100, 200, 300, 400, 500, 600, 700, 800, 900, 1000, 2,000 or even 3,000 times greater than the cell density of the stem cells in the initial cochlear cell population. In each of the foregoing embodiments, the capacity of a compound or composition of the present disclosure to expand a cochlear cell population as described in this paragraph may be determined by means of a Stem Cell Proliferation Assay.

In accordance with one aspect of the present disclosure, a cochlea supporting cell population or vestibular supporting cell population is treated with a compound or composition provided herein to increase the Lgr5 activity of the population. For example, in some embodiments the compound or composition provided herein has the capacity to increase and maintain the Lgr5 activity of an in vitro population of cochlea supporting cells or vestibular supporting cells by factor of at least 1.2. By way of further example, in one such embodiment the compound or composition has the capacity to increase the Lgr5 activity of an in vitro population of cochlea supporting cells or vestibular supporting cells by factor of 1.5. By way of further example, in one such embodiment the compound or composition has the capacity to increase the Lgr5 activity of an in vitro population of cochlea supporting cells or vestibular supporting cells by factor of 2, 3, 5 10, 100, 500, 1,000, 2,000 or even 3,000. Increases in Lgr5 activity may also be observed for in vivo populations but the observed increase may be somewhat more modest. For example, in some embodiments the compound or composition has the capacity to increase the Lgr5 activity of an in vivo population of cochlea supporting cells or vestibular supporting cells by at least 5%. By way of further example, in one such embodiment the compound or composition has the capacity to increase the Lgr5 activity of an in vivo population of cochlea supporting cells or vestibular supporting cells by at least 10%. By way of further example, in one such embodiment the compound or composition has the capacity to increase the Lgr5 activity of an in vivo population of cochlea supporting cells or vestibular supporting cells by at least 20%. By way of further example, in one such embodiment the compound or composition has the capacity to increase the Lgr5 activity of an in vivo population of cochlea supporting cells or vestibular supporting cells by at least 30%. In each of the foregoing embodiments, the capacity of the compound or composition for such an increase in Lgr5 activity may be demonstrated, for example, in an In Vitro Lgr5+ Activity Assay and in an in vivo population may be demonstrated, for example, in an In Vivo Lgr5+ Activity Assay, as measured by isolating the organ and performing morphological analyses using immunostaining, endogenous fluorescent protein expression of Lgr5 (eg. Lgr5, Sox2), and qPCR for Lgr5.

In addition to increasing the Lgr5 activity of the population, the number of Lgr5+ supporting cells in a cochlea cell population or vestibular cell population may be increased by treating a cochlea cell population or vestibular cell population containing Lgr5+ supporting cells (whether in vivo or in vitro) with a compound or composition provided herein. In general, the cell density of the stem/progenitor supporting cells may expand relative to the initial cell population via one or more of several mechanisms. For example, in one such embodiment, newly generated Lgr5+ supporting cells may be generated that have increased stem cell propensity (i.e., greater capacity to differentiate into hair cell). By way of further example, in one such embodiment no daughter Lgr5+ cells are generated by cell division, but pre-existing Lgr5+ supporting cells are induced to differentiate into hair cells. By way of further example, in one such embodiment no daughter cells are generated by cell division, but Lgr5− supporting cells are activated to a greater level of Lgr5 activity and the activated supporting cells are then able to differentiate into hair cells. Regardless of the mechanism, in some embodiments the compound or composition of the present disclosure has the capacity to increase the cell density of Lgr5+ supporting cells in an in vitro isolated cell population of cochlea supporting cells or vestibular supporting cells by a factor of at least 5. By way of further example, in one such embodiment the compound or composition has the capacity to increase the cell density of Lgr5+ supporting cells or vestibular supporting cells in an in vitro population of cochlea supporting cells by a factor of at least 10. By way of further example, in one such embodiment the compound or composition has the capacity to increase the cell density of Lgr5+ supporting cells in an in vitro population of cochlea supporting cells or vestibular supporting cells by a factor of at least 100, at least 500, at least 1,000 or even at least 2,000. Increases in the cell density of Lgr5+ supporting cells may also be observed for in vivo populations but the observed increase may be somewhat more modest. For example, in some embodiments the compound or composition has the capacity to increase the cell density of Lgr5+ supporting cells in an in vivo population of cochlea supporting cells or vestibular supporting cells by at least 5%. By way of further example, in one such embodiment the compound or composition has the capacity to increase the cell density of Lgr5+ supporting cells in an in vivo population of cochlea supporting cells or vestibular supporting cells by at least 10%. By way of further example, in one such embodiment the compound or composition has the capacity to increase the cell density of Lgr5+ supporting cells in an in vivo population of cochlea supporting cells or vestibular supporting cells by at least 20%. By way of further example, in one such embodiment the compound or composition has the capacity to increase the cell density of Lgr5+ supporting cells in an in vivo population of cochlea supporting cells or vestibular supporting cells by at least 30%. The capacity of the compound or composition for such an increase in Lgr5+ supporting cells in an in vitro population may be demonstrated, for example, in a Stem Cell Proliferation Assay or in an appropriate in vivo assay. In some embodiments, a compound or composition of the present disclosure has the capacity to increase the number of Lgr5+ cells in the cochlea by inducing expression of Lgr5 in cells with absent or low detection levels of the protein, while maintaining Native Morphology. In some embodiments, a compound or composition of the present disclosure has the capacity to increase the number of Lgr5+ cells in the cochlea by inducing expression of Lgr5 in cells with absent or low detection levels of the protein, while maintaining Native Morphology and without producing Cell Aggregates.

In addition to increasing the cell density of Lgr5+ supporting cells, in some embodiments the method of the present disclosure has the capacity to increase the ratio of Lgr5+ cells to hair cells in a cochlear cell population. In some embodiments, the number of Lgr5+ supporting cells in an initial cochlear cell population is selectively expanded by treating the initial cochlear cell population with a compound or composition of the present disclosure to form an expanded cell population and wherein the number of Lgr5+ supporting cells in the expanded cochlear cell population at least equals the number of hair cells. The expanded cochlear cell population may be, for example, an in vivo population, an in vitro population or even an in vitro explant. In one such embodiment, the ratio of Lgr5+ supporting cells to hair cells in the expanded cochlear cell population is at least 1:1. For example, in one such embodiment the ratio of Lgr5+ supporting cells to hair cells in the expanded cochlear cell population is at least 1.5:1. By way of further example, in one such embodiment the ratio of Lgr5+ supporting cells to hair cells in the expanded cochlear cell population is at least 2:1. By way of further example, in one such embodiment the ratio of Lgr5+ supporting cells to hair cells in the expanded cochlear cell population is at least 3:1. By way of further example, in one such embodiment the ratio of Lgr5+ supporting cells to hair cells in the expanded cochlear cell population is at least 4:1. By way of further example, in one such embodiment the ratio of Lgr5+ supporting cells to hair cells in the expanded cochlear cell population is at least 5:1. In each of the foregoing embodiments, the capacity of the compound or composition of the present disclosure to expand a cochlear cell population as described in this paragraph may be determined by means of a Stem Cell Proliferation Assay.

Among the various aspects of the present disclosure, therefore, may be noted a method for activating a cell cycle progression pathway in a cell population to increase the capacity of the population for self-renewal, i.e., the capacity for repeated generation of daughter cells with equivalent proliferation and ‘cell fate specification’ potential, and differentiation, i.e., the capacity for generation of daughter cells specified for differentiation. In some embodiments, the cell population is a cochlear supporting cell population. In some embodiments, the pathway is activated without any genetic modification of the population. In some embodiments, the pathway is activated by small molecules that transiently induce such activity. In some embodiments, the supporting cell population includes supporting cells that are LGR5+ and endogenous to the organ of Corti.

A further aspect of the present disclosure is a method for inducing the self-renewal of stem/progenitor supporting cells comprised by a cochlear cell population. That is, the stem/progenitor supporting cells are induced to proliferate (i.e., divide and form daughter cells) while maintaining, in the daughter cells, the capacity to differentiate into hair cells. In contrast, if the stem/progenitor supporting cells were merely induced to proliferate (without maintaining multi-potency), the daughter cells would lack the capacity to divide into hair cells. Further, merely enforcing differentiation of a pre-existing stem/progenitor cell population has the potential to exhaust the stem cell pool. In some embodiments, proliferation is activated by small molecules that transiently induce such activity. Additionally, in some embodiments the supporting cell population includes supporting cells that are LGR5+ and endogenous to the organ of Corti.

In some aspects, the present disclosure provides a method of using a compound disclosed herein for inducing the self-renewal of stem/progenitor supporting cells. In some embodiments, the compound is of Formula (I) or a pharmaceutically acceptable salt thereof.

In some embodiments, therefore, the present disclosure provides methods to induce self-renewal of a population of supporting cells by activating pathways and mechanisms that are known to be involved in inducing stem cell properties, such as those used to create “induced pluripotent stem cells”. In some embodiments, the pathways are activated with small molecules. For example, a compound when applied in vitro to a supporting cell population induces the population to proliferate to a high degree and in high purity in a Stem Cell Proliferation Assay, and also allows the population to differentiate into a high purity population of a tissue cell in a Stem Cell Differentiation Assay. In one such embodiment, the compound induces and maintains stem cell properties by proliferating to produce stem cells that can divide for many generations and maintain the ability to have a high proportion of the resulting cells differentiate into tissue cells. Further, the proliferating stem cells express stem cell markers which may include one or more of Lgr5, Sox2, Opem1, Phex, lin28, Lgr6, cyclin D1, Msx1, Myb, Kit, Gdnf3, Zic3, Dppa3, Dppa4, Dppa5, Nanog, Esrrb, Rex1, Dnmt3a, Dnmt3b, Dnmt31, Utf1, Id 1, Oct4, Klf4, Pax6, Six2, Zic1, Zic2, Otx2, Bmi1, CDX2, STAT3, Smad1, Smad2, smad2/3, smad4, smad5, and smad7.

In some embodiments, the disclosure provides a method for expanding a population of cochlear cells in a cochlear tissue comprising a parent population of cells. In this embodiment, the method comprises contacting the cochlear tissue with a stem cell proliferator to form an expanded population of cells in the cochlear tissue, wherein

the stem cell proliferator is capable of (i) forming a proliferation assay final cell population from a proliferation assay initial cell population over a proliferation assay time period in a stem cell proliferation assay and (ii) forming a differentiation assay final cell population from a differentiation assay initial cell population over a differentiation assay time period in a stem cell differentiation assay wherein:

(a) the proliferation assay initial cell population has (i) a proliferation assay initial number of total cells, (ii) a proliferation assay initial number of Lgr5⁺ cells, (iii) a proliferation assay initial number of hair cells, (iv) a proliferation assay initial Lgr5⁺ cell fraction that equals the ratio of the proliferation assay initial number of Lgr5⁺ cells to the proliferation assay initial number of total cells, and (v) a proliferation assay initial hair cell fraction that equals the ratio of the proliferation assay initial number of hair cells to the proliferation assay initial number of total cells;

(b) the proliferation assay final cell population has (i) a proliferation assay final number of total cells, (ii) a proliferation assay final number of Lgr5⁺ cells, (iii) a proliferation assay final number of hair cells, (iv) a proliferation assay final Lgr5⁺ cell fraction that equals the ratio of the proliferation assay final number of Lgr5⁺ cells to the proliferation assay final number of total cells and (v) a proliferation assay final hair cell fraction that equals the ratio of the proliferation assay final number of hair cells to the proliferation assay final number of total cells;

(c) the differentiation assay initial cell population has (i) a differentiation assay initial number of total cells, (ii) a differentiation assay initial number of Lgr5⁺ cells, (iii) a differentiation assay initial number of hair cells, (iv) a differentiation assay initial Lgr5⁺ cell fraction that equals the ratio of the differentiation assay initial number of Lgr5⁺ cells to the differentiation assay initial number of total cells, and (v) a differentiation assay initial hair cell fraction that equals the ratio of the differentiation assay initial number of hair cells to the differentiation assay initial number of total cells;

(d) the differentiation assay final cell population has (i) a differentiation assay final number of total cells, (ii) a differentiation assay final number of Lgr5⁺ cells, (iii) a differentiation assay final number of hair cells, (iv) a differentiation assay final Lgr5⁺ cell fraction that equals the ratio of the differentiation assay final number of Lgr5⁺ cells to the differentiation assay final number of total cells, and (v) a differentiation assay final hair cell fraction that equals the ratio of the differentiation assay final number of hair cells to the differentiation assay final number of total cells;

(e) the proliferation assay final number of Lgr5⁺ cells exceeds the proliferation assay initial number of Lgr5⁺ cells by a factor of at least 10; and

(f) the differentiation assay final number of hair cells is a non-zero number.

In some embodiments, the assay does not include applying an additional activator or an additional inhibitor.

In some embodiments, the disclosure provides a method for increasing the cell density of supporting cells in a population of cochlear cells. The method comprises modulating pathways and mechanisms that induce stem cell properties in the supporting cells, proliferating the activated supporting cells (while maintaining the multi-potent character of the supporting cells in the newly formed daughter cells) and thereafter allowing (or even inducing) the expanded population to differentiate into hair cells to form an expanded cochlear cell population wherein the cell density of hair cells in the expanded cochlear cell population exceeds the cell density of hair cells in the original (non-expanded) cochlear cell population. In some embodiments, such proliferation occurs in the absence of and additional activator or an additional inhibitor. In some embodiments, the supporting cell population is an in vitro supporting cell population. In some embodiments, the supporting cell population is an in vivo supporting cell population. In some embodiments, the proliferation stage is controlled to substantially maintain the native organization of the cochlear structure. The proliferation is induced by the compound described herein that transiently induces such activity rather than by induction and without any genetic modification of the population. In some embodiments, such proliferation occurs in the absence of an additional activator or an additional inhibitor. Additionally, in some embodiments the supporting cell population includes supporting cells that are LGR5⁺ and endogenous to the organ of Corti.

In some embodiments, the disclosure provides a method for increasing the cell density of Lgr5+ supporting cells in a population of cochlear cells. The method comprises modulating pathways and mechanisms that induce or maintain stem cell properties in the Lgr5+ supporting cells, proliferating the activated Lgr5+ supporting cells (while maintaining such stem cell properties) and thereafter allowing (or even inducing) the expanded population to differentiate into hair cells to form an expanded cochlear cell population wherein the cell density of hair cells in the expanded cochlear cell population exceeds the cell density of hair cells in the original (non-expanded) cochlear cell population. In some embodiments for increasing the cell density of Lgr5+ supporting cells in a population of cochlear cells, such increasing of the cell density occurs in the absence of an additional activator or an additional inhibitor. In some embodiments, the Lgr5+ supporting cell population is an in vitro Lgr5+ stem cell population. In some embodiments, the Lgr5+ supporting cell population is an in vivo supporting cell population. Additionally, in some embodiments the proliferation stage is controlled to substantially maintain the native organization of the cochlear structure.

In some embodiments, the disclosure provides a method for increasing the cell density of hair cells in an initial population of cochlear cells, the initial population (which may be an in vivo or an in vitro population) comprises hair cells, Lgr5− supporting cells, and Lgr5+ supporting cells. In some embodiments for increasing the cell density of hair cells in an initial population of cochlear cells, such increasing of the cell density occurs in the absence of an additional activator or an additional inhibitor. The method comprises administering to the initial population a compound described herein.

In some embodiments, the method produces stem cells in a Stem Cell Proliferation Assay that express stem cells markers Lgr5+. In some embodiments, if a mixed population of Lgr5+ and non-Lgr5+ stems are placed in a Stem Cell Proliferation Assay, the method increases the fraction of cells in the population that are Lgr5+. In some embodiments, such production of stem cells in a Stem Cell Proliferation Assay occurs in the absence of an additional activator or an additional inhibitor.

Expanding supporting cell populations to a degree that destroys the native organization of the cochlear structure could inhibit cochlear function. Driving proliferation of existing supporting cells with a small molecule signal may allow for a more controlled regeneration of hair cells than using gene delivery, which is incapable of targeting a specific cell type and permanently alters a cell's genetic information. An approximately normal cochlear structure is desired with rows of hair cells that have supporting cells between them, and hair cells do not contact other hair cells. Further, it would be desirable to avoid using genetic modification to drive proliferation to create large cell aggregations in the cochlea that disrupt the organ's anatomy.

In some embodiments, the disclosure provides a method for increasing the cell density of hair cells in an initial population of cochlear cells comprising hair cells and supporting cells. The method comprises selectively expanding the number of supporting cells in the initial population to form an intermediate cochlear cell population wherein the ratio of the number of supporting cells to hair cells in the intermediate cochlear cell population exceeds the ratio of the number of supporting cells to hair cells in the initial cochlear cell population. The method further comprises generating hair cells in the intermediate cochlear cell population to form an expanded cochlear cell population wherein the ratio of the number of hair cells to supporting cells in the expanded cochlear cell population exceeds the ratio of the number of hair cells to supporting cells in the intermediate cochlear cell population. In some embodiments, the method does not comprise the use of an additional activator or an additional inhibitor.

In some embodiments, the disclosure provides a method for increasing the number of Lgr5+ supporting cells or increasing the Lgr5+ activity in an initial population of cochlear cells, wherein the initial population comprises supporting cells and hair cells. For example, in one such method an intermediate population is formed in which the number of Lgr5+ supporting cells is expanded relative to the initial population. Alternatively, in one such method an intermediate population is formed in which the Lgr5+ activity of the supporting cells relative to the initial population is increased. Alternatively, a method where the number of Lgr5+ cells is increased relative to the initial cell population by activating Lgr5+ expression in cell types that normally lack or have very low levels of Lgr5+. In some embodiments, these alternative methods do not comprise the use of an additional activator or an additional inhibitor. By way of further example, an intermediate population is formed in which the number of Lgr5+ supporting cells is expanded and the Lgr5 activity is increased relative to the initial cochlear cell population. Thereafter, hair cells in the intermediate cochlear cell population may be generated to form an expanded cochlear cell population wherein the ratio of hair cells to supporting cells in the expanded cochlear cell population exceeds the ratio of the number of hair cells to supporting cells in the intermediate cochlear cell population.

In each of the aforementioned embodiments of the present disclosure, sternness is induced by inhibiting one or more of FOXO-1, GSK3 α/β and LSD-1 activity. In some embodiments, inducing stemness does not comprise the use of an additional activator or an additional inhibitor.

In some embodiments, the disclosure provides methods for preventing and treating auditory dysfunction. For example, in some embodiments, the disclosure provides methods for preventing or treating auditory impairments in a subject comprising administering to said subject an effective amount of a compound or composition provided herein.

In some embodiments, the present disclosure also relates to ex-vivo uses of cells described herein. For example, approaches described herein can be used for high throughput screen and for discovery purposes. For example, some embodiments of the present disclosure are useful for identifying agents that proliferate hair cell progenitors and/or increase numbers of hair cells, and also agents that protect supporting cells and/or hair cells (e.g., to support their survival), and also for identifying agents that are toxic or not toxic to supporting cells or differentiated progeny including hair cells.

In some embodiments, the present disclosure further provides methods for treating or preventing diseases that may be ameliorated by regenerative medicine therapies such as stem cell therapy. Exemplary diseases include, but not limited to, Friedreick's ataxia, heart disease, vascular degeneration, prophylaxis and/or acute and chronic ear disease and hearing loss, vestibular diseases, dizziness and balance problems especially of sudden hearing loss, acoustic trauma, hearing loss due to chronic noise exposure, presbycusis, trauma during implantation of the inner ear prosthesis (insertion trauma), dizziness due to diseases of the inner ear area, dizziness related and/or as a symptom of Meniere's disease, vertigo related and/or as a symptom of Meniere's disease, tinnitus, and hearing loss due to antibiotics, cytostatics and/or other drugs

In some embodiments, the method is a treatment for promoting the repair damaged mucosa related to diseases such as chemotherapy-induced gastrointestinal mucositis, Graph Versus Host Disease, gastric ulcer, Crohns, or ulcerative colitis

In some embodiments, the compounds and/or compositions of the present disclosure may be useful for the treatment or prevention of LSD-1-mediated diseases or disorders as well as diseases or disorders where LSD-1 plays a role. Exemplary diseases and disorders include, but are not limited to, hematologic malignancies, solid tumors, B cell lymphoma, acute myeloid leukemia, gastric cancer, hepatocellular carcinoma, prostate cancer, breast carcinoma, neuroblastoma, glioblastoma, nasopharyngeal carcinoma, colon cancer, gallbladder cancer, esophageal cancer, head and neck cancer, lung cancer, ovarian cancer, pancreatic cancer, endometrial carcinoma, and soft tissue sarcomas such as rhabdomyosarcoma (RMS), chondrosarcoma, osteosarcoma, Acute Myeloid Leukemia, Ewing's sarcoma, liver fibrosis, and sickle cell disease.

In some embodiments, the compounds and/or compositions of the present disclosure may be useful for the treatment or prevention of GSK3 alpha/beta-mediated diseases or disorders as well as diseases or disorders where GSK3 alpha/beta plays a role. Exemplary diseases and disorders include, but are not limited to, stem cell therapy, cancer, alopecia, Type II Diabetes, Obesity, Alzheimer's disease, Parkinson's, mood disorders, schizophrenia, bipolar disorder, Osteoporosis, Atherosclerosis, Cardiac Hypertrophy, Down syndrome, bacterial or viral infections, reduced infarct volume, inflammatory diseases, insult-induced neuroinflammation, inflammatory mediated diseases, such as depression, bipolar disorders, and platelet aggregation therapy.

In some embodiments, the compounds and/or compositions of the present disclosure may be useful for the treatment or prevention of FOXO-1-mediated diseases or disorders as well as diseases or disorders where FOXO-1 plays a role. Exemplary diseases and disorders include, but are not limited to, Type II Diabetes, Obesity, and bacterial or viral infections.

In some embodiments, the disclosure provides for methods for inhibiting the loss or death of the cells of the auditory system in a subject comprising administering to said subject an effective amount of the compound described herein or derivative thereof or pharmaceutically acceptable salt thereof and an acceptable carrier or excipient, thereby inhibiting loss or death of the cells of the auditory system in the subject. In some embodiments, the method does not comprise the use of an additional activator or an additional inhibitor.

In some embodiments, the disclosure provides methods for maintaining or promoting the growth of cells of the auditory system in a subject comprising administering to said subject the compound described herein or derivative thereof or pharmaceutically acceptable salt thereof in an effective amount so as to augment or initiate endogenous repair, thereby maintaining or promoting the growth of cells of the auditory system in the subject.

Also described herein is a method for expanding a population of cochlear cells in a cochlear tissue comprising a parent population of cells, the parent population including supporting cells and a number of Lgr5+ cells, the method comprising contacting the cochlear tissue with a stem cell proliferator to form an expanded population of cells in the cochlear tissue, wherein the stem cell proliferator is capable (i) in a stem cell proliferation assay of increasing the number of Lgr5+ cells in a stem cell proliferation assay cell population by a factor of at least 10 and (ii) in a stem cell differentiation assay of forming hair cells from a cell population comprising Lgr5+ cells. In some embodiments for expanding a population of cochlear cells, the method does not comprise the use of an additional activator or an additional inhibitor.

Also described herein is a method for expanding a population of cochlear cells in a cochlear tissue comprising a parent population of cells, the parent population including supporting cells, the method comprising contacting the cochlear tissue with a stem cell proliferator to form an expanded population of cells in the cochlear tissue. The stem cell proliferator can be capable of (i) forming a proliferation assay final cell population from a proliferation assay initial cell population over a proliferation assay time period in a stem cell proliferation assay and (ii) forming a differentiation assay final cell population from a differentiation assay initial cell population over a differentiation assay time period in a stem cell differentiation assay wherein: (a) the proliferation assay initial cell population has (i) a proliferation assay initial number of total cells, (ii) a proliferation assay initial number of Lgr5+ cells, (iii) a proliferation assay initial number of hair cells, (iv) a proliferation assay initial Lgr5+ cell fraction that equals the ratio of the proliferation assay initial number of Lgr5+ cells to the proliferation assay initial number of total cells, and (v) a proliferation assay initial hair cell fraction that equals the ratio of the proliferation assay initial number of hair cells to the proliferation assay initial number of total cells; (b) the proliferation assay final cell population has (i) a proliferation assay final number of total cells, (ii) a proliferation assay final number of Lgr5+ cells, (iii) a proliferation assay final number of hair cells, (iv) a proliferation assay final Lgr5+ cell fraction that equals the ratio of the proliferation assay final number of Lgr5+ cells to the proliferation assay final number of total cells and (v) a proliferation assay final hair cell fraction that equals the ratio of the proliferation assay final number of hair cells to the proliferation assay final number of total cells; (c) the differentiation assay initial cell population has (i) a differentiation assay initial number of total cells, (ii) a differentiation assay initial number of Lgr5+ cells, (iii) a differentiation assay initial number of hair cells, (iv) a differentiation assay initial Lgr5+ cell fraction that equals the ratio of the differentiation assay initial number of Lgr5+ cells to the differentiation assay initial number of total cells, and (v) a differentiation assay initial hair cell fraction that equals the ratio of the differentiation assay initial number of hair cells to the differentiation assay initial number of total cells; (d) the differentiation assay final cell population has (i) a differentiation assay final number of total cells, (ii) a differentiation assay final number of Lgr5+ cells, (iii) a differentiation assay final number of hair cells, (iv) a differentiation assay final Lgr5+ cell fraction that equals the ratio of the differentiation assay final number of Lgr5+ cells to the differentiation assay final number of total cells, and (v) a differentiation assay final hair cell fraction that equals the ratio of the differentiation assay final number of hair cells to the differentiation assay final number of total cells; (e) the proliferation assay final number of Lgr5+ cells exceeds the proliferation assay initial number of Lgr5+ cells by a factor of at least 10; and (f) the differentiation assay final number of hair cells is a non-zero number. In some embodiments of the assay described above, the assay does not comprise the use of an additional activator or an additional inhibitor.

The proliferation assay final number of Lgr5+ cells can be greater than the proliferation assay initial number of Lgr5+ cells by a factor of at least 50, or by a factor of at least 100. The expanded population of cells in the cochlear tissue can include a greater number of hair cells than does the parent population. The proliferation assay final Lgr5+ cell fraction can be greater than the differentiation assay initial Lgr5+ cell fraction by at least a factor of 2. The differentiation assay final hair cell fraction can be greater than the proliferation assay initial hair cell fraction by at least a factor of 2. The proliferation assay final hair cell fraction can be at least 25% less than the proliferation assay initial hair cell fraction. The proliferation assay final Lgr5+ cell fraction can be at least 10% greater than proliferation assay initial Lgr5+ cell fraction. One of more morphological characteristics of the cochlear tissue can be maintained. Native morphology can be maintained. The stem cell proliferator can be dispersed in a biocompatible matrix, which can be a biocompatible gel or foam. The cochlear tissue can be an in vivo cochlear tissue or an ex vivo cochlear tissue. The method can produce a population of Lgr5+ cells that are in s-phase. The cochlear tissue can be in a subject, and contacting the cochlear tissue with the compound can be achieved by administering the compound trans-tympanically to the subject. Contacting the cochlear tissue with the compound can result in improved auditory functioning of the subject.

Also described herein is a method of treating or preventing hearing loss in a subject in need thereof. The method can include trans-tympanically administering to the subject (e.g., to a cochlear tissue of the subject) compound provided herein. In some embodiments, the hearing loss is sensorineural hearing loss.

Also described herein is a method of generating Myo7a+ cochlear cells. The method can include contacting Lgr5+ cochlear cells with a compound provided herein, thereby generating an expanded population of Lgr5+ cells, thereby generating Myo7a+ cochlear cells.

In some embodiments, the method increases the fraction of the Lgr5+ cells to total cells on the sensory epithelium by at least 10%, 20%, 50%, 100%, 250% 500%, 1,000% or 5000%.

In some embodiments, the method increases the Lgr5+ cells until they become at least 10, 20, 30, 50, 70, or 85% of the cells on the sensory epithelium, e.g., the organ of Corti.

In some embodiments, excessive proliferation of supporting cells in the cochlea is avoided. In some embodiments, the method of the present disclosure has the capacity to expand a cochlear cell population without creating a protrusion of new cells beyond the native surface of the cochlea, e.g a Cell Aggregate. In some embodiments, 30 days after placing a compound or composition provided herein on the round or oval membrane, the cochlear tissue has Native Morphology. In some embodiments, 30 days after placing the compound or composition on the round or oval membrane, the cochlear tissue has Native Morphology and lacks Cell Aggregates. In some embodiments, 30 days after placing the compound or composition on the round or oval membrane, the cochlear tissue has Native Morphology and at least 10, 20, 30, 50, 75, 90, 95, 98, or even at least 99% of the Lgr5+ cells in the organ of Corti are not part of Cell Aggregates.

In addition to expanding supporting cell populations, generally, and Lgr5+ supporting cells, specifically, as described above, the method of the present disclosure has the capacity to maintain, in the daughter cells, the capacity to differentiate into hair cells. In in vivo populations, the maintenance of this capacity may be indirectly observed by an improvement in a subject's hearing. In in vitro populations, the maintenance of this capacity may be directly observed by an increase in the number of hair cells relative to a starting population or indirectly by measuring LGR5 activity, SOX2 activity or one or more of the other stem cell markers identified elsewhere herein.

In some embodiments, the capacity of the method to increase the stemness of a population of cochlear supporting cells, in general, or a population of Lgr5+ supporting cells, in particular, may be correlated with an increase of Lgr5 activity of an in vitro population of isolated Lgr5+ cells as determined by an Lgr5 Activity Assay. As previously noted, in one such embodiment, the compound or composition has the capacity to increase the Lgr5 activity of stem cells in the intermediate cell population by a factor of 5 on average relative to the Lgr5 activity of the cells in the initial cell population. By way of further example, in one such embodiment the method has the capacity to increase the Lgr5 activity of the stem cells genes in the intermediate cell population by a factor of 10 relative to the Lgr5 activity of the cells in the initial cell population. By way of further example, in one such embodiment the method has the capacity to increase the Lgr5 activity of the stem cells in the intermediate cell population by a factor of 100 relative to the Lgr5 activity of the cells in the initial cell population. By way of further example, in one such embodiment the method has the capacity to increase the Lgr5 activity of the stem cells in the intermediate cell population by a factor of 1000 relative to the Lgr5 activity of the cells in the initial cell population. In each of the foregoing embodiments, the increase in the activity of stem cells in the cell population may be determined in vitro by immunostaining or endogenous fluorescent protein expression for target genes and analysis of their relative intensities via imaging analysis or flow cytometry, or using qPCR for target stem cell genes. The identity of the resulting stem cell population may optionally be further determined by stem cell assays including stem cell marker expression assay, colony forming assay, self-renewal assay and differentiation assay as defined in Stem cell assay.

In some embodiments, the method applied to an adult mammal produces a population of adult mammalian Lgr5+ cells that are in S-phase.

In some embodiments, after applying the compound or composition provided herein to the round or oval of a mouse, the in vivo Lgr5+ Activity of a cell population in the organ of Corti increases 1.3×, 1.5×, up to 20× over baseline for a population that has not been exposed to the compound or composition. In some embodiments, applying the compound or composition to the round or oval of a mouse increases the average In vivo Lgr5+ Activity for cells in the organ of Corti is increased 1.3×, 1.5×, up to 20× over baseline for a population that has not been exposed to the compound or composition.

In some embodiments, the method increases the Lgr5+ cells until they become at least 10%, 7.5%, 10%, up to 100% of the supporting cell population by number.

In some embodiments, the compound or composition has the capacity to increase the percentage of Lgr5+ cell in a cochlea by 5%, 10%, 25%, 50%, or 80%.

In some embodiments, the stem cell population is of an in vivo subject, and the method is a treatment for hearing loss and/or vestibular dysfunction (e.g., wherein the generation of inner ear hair cells from the expanded population of stem cells results in partial or full recovery of hearing loss and/or improved vestibular function). In some embodiments, the stem cell population is of an in vivo subject, and the method further comprises delivering a drug or active pharmaceutical agent to the subject (e.g., for treatment of a disease and/or disorder unrelated to hearing loss and/or vestibular dysfunction) at a higher concentration than a known safe maximum dosage of the drug or active pharmaceutical agent for the subject (e.g., the known safe maximum dosage if delivered in the absence of the generation of inner ear hair cells resulting from the method) (e.g., due to a reduction or elimination of a dose-limiting ototoxicity of the drug).

In some embodiments, the method further comprises performing high throughput screening using the generated inner ear hair cells. In some embodiments, the method comprises using the generated inner ear hair cells to screen molecules for toxicity against inner ear hair cells. In some embodiments, the method comprises using the generated inner ear hair cells to screen molecules for ability to improve survival of inner ear hair cells (e.g., inner ear hair cells exposed to said molecules).

In another aspect, the disclosure is directed to a method of producing an expanded population of stem cells, the method comprising: administering or causing to be administered to a stem cell population (e.g., of an in vitro, ex vivo, or in vivo sample/subject) a compound or composition provided herein.

In some embodiments, the administering step is carried out by performing one or more injections into the ear (e.g., transtympanically into the middle ear and/or inner ear).

In some embodiments, the administering step comprises administering the FOXO-1, GSK3 α/β and/or LSD-1 inhibitor and/or cell cycle progression pathway agonist in a sustained manner.

In some embodiments, the stem cells are inner ear stem cells and/or supporting cells.

In some embodiments, the method further comprises performing high throughput screening using the generated expanded population of stem cells. In some embodiments, the method further comprises using the generated stem cells to screen molecules for toxicity against stem cells and by their progeny. In some embodiments, the method comprises using the generated stem cells to screen molecules for ability to improve survival of stem cells and/or their progeny.

In another aspect, the disclosure is directed to a method of treating or preventing hearing loss and/or vestibular dysfunction in a subject in need thereof: identifying a subject who has experienced, or is at risk for developing, hearing loss and/or vestibular dysfunction, administering or causing to be administered a compound or composition provided herein. In some embodiments, the hearing loss is sensorineural hearing loss.

In some embodiments, the stem cell population comprises Lgr5+ cells. In some embodiments, the stem cell population comprises post-natal cells. In some embodiments, the stem cell population comprises epithelial stem cells. In some embodiments, stem cells include progenitor cells.

In some embodiments, the step of administering is carried out by performing one or more injections into the ear (e.g., transtympanically into the middle ear and/or inner ear).

In another aspect, the disclosure is directed to a method of generating inner ear hair cells, the method comprising: proliferating stem cells in an initial stem cell population (e.g., of an in vitro, ex vivo, or in vivo sample/subject), resulting in an expanded population of stem cells (e.g., such that the expanded population is a factor of at least 1.25, 1.5, 1.75, 2, 3, 5, 10, or 20 greater than the initial stem cell population); and facilitating generation of inner ear hair cells from the expanded population of stem cells.

In another aspect, the disclosure is directed to a method of generating inner ear hair cells, the method comprising administering a compound or composition provided herein (e.g., in a pharmaceutically acceptable form (e.g., salt)) to a cell population in an inner ear of a subject, thereby facilitating generation of inner ear hair cells.

In another aspect, the disclosure is directed to a method of generating inner ear hair cells, the method comprising: proliferating post-natal LGR5⁺ cells in an initial population (e.g., of an in vitro, ex vivo, or in vivo sample/subject), resulting in an expanded population of Lgr5+ cells (e.g., such that the expanded population is a factor of at least 1.25, 1.5, 1.75, 2, 3, 5, 10, or 20 greater than the initial stem cell population), said expanded population of Lgr5+ cells resulting in generation of inner ear hair cells. In some embodiments, stem cells include progenitor cells.

In another aspect, the disclosure is directed to a method of treating or preventing a disease or disorder, the method comprising: proliferating post-natal Lgr5+ epithelial cells in an initial population of a subject (in vivo), resulting in an expanded population of Lgr5+ epithelial cells (e.g., such that the expanded population is a factor of at least 1.25, 1.5, 1.75, 2, 3, 5, 10, or 20 greater than the initial post-natal Lgr5+ epithelial cell population).

In some embodiments, Lgr5+ cells are differentiated into hair cells.

In some aspects, the present disclosure provides a method for proliferation of stem cells comprising administering to a cell population an effective amount of a compound of the present disclosure.

In some aspects, proliferation occurs in the absence of an additional activator or an additional inhibitor.

In some aspects, the present disclosure provides a compound of the present disclosure, or a pharmaceutically acceptable salt or tautomer thereof, for use in treating or preventing a disease associated with absence or lack of certain tissue cells in a subject in need thereof.

In some aspects, the present disclosure provides a compound of the present disclosure, or a pharmaceutically acceptable salt or tautomer thereof, for use in treating or preventing hearing loss in a subject in need thereof. In some embodiments, the hearing loss is sensorineural hearing loss.

In some aspects, the present disclosure provides a use of a compound of the present disclosure, or a pharmaceutically acceptable salt or tautomer thereof, in the manufacture of a medicament for treating or preventing a disease associated with absence or lack of certain tissue cells in a subject in need thereof.

In some aspects, the present disclosure provides a use of a compound of the present disclosure, or a pharmaceutically acceptable salt or tautomer thereof, in the manufacture of a medicament for treating or preventing hearing loss in a subject in need thereof. In some embodiments, the hearing loss is sensorineural hearing loss.

In some aspects, the present disclosure provides a use of a compound of the present disclosure, or a pharmaceutically acceptable salt or tautomer thereof, in the manufacture of a medicament for treating or preventing a disease responding to LSD inhibition, GSK3 inhibition, and/or FOXO inhibition in a subject in need thereof.

In some aspects, the present disclosure provides a use of a compound of the present disclosure, or a pharmaceutically acceptable salt or tautomer thereof, in the manufacture of a medicament for treating or preventing vestibular diseases, alopecia, oncology, acute myeloid leukemia, inflammation, Alzheimer's disease, Huntington's disease, Friedreick's ataxia, depression, anxiety, manic episodes of bipolar/mood disorders, Parkinson's, diabetes, bacterial infection, Anti-Trypanosoma brucei, ischemia, heart disease, vascular degeneration, and/or platelet aggregation in a subject in need thereof.

All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of compounds disclosed herein, the chemical structures will control.

Other objects, features, and advantages will be in part apparent and in part pointed out hereinafter from the following detailed description and claims.

### Hair Cell Regrowth

In some embodiments, the present disclosure is directed to a method of facilitating the generation of inner ear hair cells, the method comprising: administering a compound or composition of present disclosure to expand the stem cell population of cochlear tissue.

In some embodiments, the present disclosure is directed to a method of facilitating the generation of inner ear hair cells, the method comprising: administering a compound or composition comprising a compound or composition of present disclosure and one or more additional pharmaceutically active agents (e.g., an HDAC inhibitor and/or a poloxamer) to expand the stem cell population of cochlear tissue.

In some embodiments, the present disclosure is directed to a method to regenerate hearing in a mammal.

In some embodiments, the stem cell population is of an in vivo subject, and the method is a treatment for hearing loss and/or vestibular dysfunction.

In some embodiments, the present disclosure is directed to a method of generating inner ear hair cells using of a compound or composition of the present disclosure to proliferate Lgr5+ cells in an initial population in vivo, resulting in an expanded population of Lgr5+ cells (e.g., such that the expanded population is at least 2 times, 3 times, 5 times, 10 times, or 20 times greater than the initial stem cell population), resulting in generation of inner ear hair cells.

In some embodiments, the present disclosure is directed to a method of generating inner ear hair cells using of a compound or composition comprising a compound or composition of present disclosure and one or more additional pharmaceutically active agents (e.g., an HDAC inhibitor and/or a poloxamer) to proliferate Lgr5+ cells in an initial population in vivo, resulting in an expanded population of Lgr5+ cells (e.g., such that the expanded population is at least 2 times, 3 times, 5 times, 10 times, or 20 times greater than the initial stem cell population), resulting in generation of inner ear hair cells.

### Intestinal Regeneration

In some embodiments, the present disclosure is directed to a method of facilitating the generation of intestinal cells, the method comprising: administering a compound or composition of the present disclosure to expand the stem cell population of intestinal epithelia.

In some embodiments, the present disclosure is directed to a method of facilitating the generation of intestinal cells, the method comprising: administering a compound or composition comprising a compound or composition of present disclosure and one or more additional pharmaceutically active agents (e.g., an HDAC inhibitor and/or a poloxamer) to expand the stem cell population of intestinal epithelia.

In some embodiments, the present disclosure is directed to a method to regenerate intestinal epithelia in a mammal.

In some embodiments, the stem cell population is of an in vivo subject. In some embodiments, the method is a treatment for promoting the repair of damaged mucosa related to diseases such as chemotherapy-induced gastrointestinal mucositis, Graph Versus Host Disease, gastric ulcer, Crohns, or ulcerative colitis.

### Intestinal Lgr5⁺ Proliferation

In some embodiments, the present disclosure is directed to a method of facilitating the generation of intestinal cells, the method comprising: administering a compound or composition of the present disclosure to expand the Lgr5+ cell population of intestinal epithelia.

In some embodiments, the present disclosure is directed to a method of facilitating the generation of intestinal cells, the method comprising: administering a compound or composition comprising a compound or composition of present disclosure and one or more additional pharmaceutically active agents (e.g., an HDAC inhibitor and/or a poloxamer) to expand the Lgr5+ cell population of intestinal epithelia.

In some embodiments, the present disclosure is directed to a method to regenerate Lgr5+ cell population intestinal cells in a mammal.

In some embodiments, the Lgr5+ cell population is in an in vivo subject. In some embodiments, the method is a treatment for promoting the repair of damaged mucosa related to diseases such as chemotherapy-induced gastrointestinal mucositis, Graph Versus Host Disease, gastric ulcer, Crohns, or ulcerative colitis.

In some embodiments, the present disclosure is directed to a method of treating or preventing a disease or disorder, the method comprising proliferating Lgr5+ epithelial cells in vivo, resulting in an expanded population of Lgr5+ epithelial cells (e.g., such that the expanded population is at least 2 times, 3 times, 5 times, 10 times, or 20 times greater than the initial post natal Lgr5+ epithelial cell population).

### Expansion of a Population of Vestibular Cells

In some embodiments, the pharmaceutical formulations containing can expand a population of vestibular cells in a vestibular tissue comprising contacting the vestibular tissue. In some embodiments, the pharmaceutical formulations are capable in a stem cell proliferation assay of increasing the number of supporting cells in a stem cell proliferation assay cell population by a factor of at least 10 or at least 50. In some embodiments, the pharmaceutical formulations are capable in a stem cell differentiation assay of forming hair cells from a cell population comprising vestibular supporting cells.

In some embodiments, the vestibular tissue maintains Native Morphology. In some embodiments, the vestibular tissue is in a subject. In some embodiments, the contacting the vestibular tissue with the compound or composition is achieved by administering the compound or composition trans-tympanically to the subject. In some embodiments, the contacting the vestibular tissue with the compound or composition results in improved vestibular functioning of the subject.

In some embodiments, the present disclosure is directed to a method of treating or preventing a disease associated with absence or lack of certain tissue cells in a subject in need thereof, the method comprising administering or causing to be administered to said subject a compound or composition of the present disclosure.

In some embodiments, the compound or composition is dispersed in a biocompatible matrix. In some embodiments, the biocompatible matrix is a biocompatible gel or foam. In some embodiments, the compound or composition is administered trans-tympanically to a vestibular tissue of the subject.

In some embodiments, the present disclosure provides a method for expanding a population of vestibular cells in a vestibular tissue comprising contacting the vestibular tissue with (i) a compound or composition of the present disclosure, and (ii) one or more additional pharmaceutically active agents (e.g., an TGF-β Inhibitor) to form an expanded population of cells in the vestibular tissue.

### Generation of Dermal Papilla Cells

In some embodiments, the present disclosure is directed to a method of facilitating generation of Dermal Papilla Cells, the method comprising: administering a compound or composition of the present disclosure, alone or in combination with one or more additional pharmaceutically active agents (e.g., an BMP inhibitor), to expand the population of Dermal Papilla Cells. In some embodiments, the compound or composition can regenerate hair in a mammal. In some embodiments, the Dermal Papilla Cells population is of an in vivo subject. In some embodiments, the Dermal Papilla Cells population is of an in vivo subject for the treatment for alopecia. In some embodiments, the present disclosure provides a method of generating Dermal Papilla Cells using of a compound or composition of the present disclosure, alone or in combination with one or more additional pharmaceutically active agents (e.g., an BMP inhibitor) to proliferate Dermal Papilla Cells in an initial population in vivo, resulting in an expanded population of Dermal Papilla Cells.

### Administration

### Middle Ear Administration

Access to the inner ear is achieved through a variety of middle-inner interface tissue structures, such as the round window membrane, the oval window/stapes footplate, the annual ligament, or the endolymphatic sac/endolymphatic duct. The membrane of the round or oval window is the biological barrier to the inner ear space and represents the major obstacle for the local treatment of hearing impairment. The administered compounds or compositions of the invention must overcome this membrane to reach the inner ear space. The compounds or compositions can be injected intra-tympanically or surgically placed in the middle ear and can then penetrate through the round window membrane. Substances (e.g., compounds) that penetrate the round window typically distribute in the perilymph and thus reach the hair cells and supporting cells.

Local administration of compounds or compositions of the invention to the inner ear via the middle ear is accomplished by various delivery techniques. These include, for example, the use of devices to transport and/or deliver the compounds or compositions of the invention in a targeted fashion to the membranes of the round or oval window, where it diffuses into the inner ear or is actively infused. Examples include otowicks (see e.g., U.S. Pat. No. 6,120,484, which is hereby incorporated by reference), round window catheters (see e.g., U.S. Pat. Nos. 5,421,818; 5,474,529; 5,476,446; 6,045,528; 6,377,849; and U.S. Pat. Pub. No. 2002/0082554, each of which is hereby incorporated by reference in its entirely for all purposes), or microimplants (see e.g., WO 2004/064912, which is hereby incorporated by reference in its entirely for all purposes). Other techniques include transtympanic injection (also referred to as “intratympanic injection”), wherein the compounds or compositions of the invention is injected through the tympanic membrane into the middle ear for diffusion across the round window membrane (see e.g., Light, J.; Silverstein, H., Curr. Opin. Otolaryngol. Head Neck Surg. 2004, 12, 378-383, which is hereby incorporated by reference in its entirely for all purposes). For repeated injections, a middle ear ventilation tube is inserted into the tympanic membrane, through which the compounds or compositions of the invention are administered to the middle ear space. Some groups have applied drugs in a sustained manner using microcatheters and microwicks, while the majority have applied them as single or as repeated IT injections (up to 8 injections over periods of up to 2 weeks). Furthermore, drug carriers that are too viscous to be injected are deposited across a small opening in the tympanic membrane with the aid of a surgical instrument.

Intratympanically-applied active agents are thought to enter the fluids of the inner ear primarily by crossing the round window (RW) and oval window (OW) membranes. Calculations show that a major factor controlling both the amount of drug entering the ear and the distribution of drug throughout the ear is the duration the drug remains in the middle ear space. Single, ‘one-shot’ applications or applications of aqueous solutions for few hours' duration result in steep drug gradients for the applied substance. Because inner ear fluids are connected, a drug delivered to the inner ear will contact the vestibular organs and cochlea.

Other injection approaches include by osmotic pump, or, by combination with implanted biomaterial, or by injection or infusion. Biomaterials that can aid in controlling release kinetics and distribution of drug include hydrogel materials, degradable materials. One class of materials that a reused includes in situ gelling materials. Other materials include collagen or other natural materials including fibrin, gelatin, and decellularized tissues. Other additives or excipients may include, for example, Gelfoam®, hyaluronic gel, Seprapack™, Poloxamer 407, chitosan glycosylated derivatives, chitosan glycerophosphate hydrogel, lipid nanocapsules, silica nanoparticles, PLGA nanoparticles, superparamagnetic iron oxide nanoparticles encapsulated PLGA nanoparticles, lipid core nanocapsules poly-L-lysine (HBPL) nanoparticles, superparamagnetic iron oxide nanoparticles, superparamagnetic iron oxide nanoparticles encapsulated pluronic F127 copolymer, polymersome, thiol-modified hyaluronic acid, glutaraldehyde cross-linking of porcine type collagen, or the like (Acta Pharmaeutica Sinica B 2013, 3(2), 86-96, which is incorporated by reference herein in its entirety for all purposes). Delivery may also be enhanced via alternate means including but not limited to agents added to the delivered composition such as penetration enhancers, or could be through devices via ultrasound, electroporation, or high speed jet.

The diffusion of pharmaceutical compounds across middle-inner ear interface tissue structures, in particular the round window membrane, depends on a variety of factors, such as molecular weight, concentration, liposolubility, electrical charge, and thickness of the membrane (Goycoolea, M.; Lundman, L., Microsc. Res. Techniq. 1997, 36, 201-211, which is incorporated by reference herein in its entirety for all purposes). Furthermore, techniques have been developed for increasing middle-inner ear diffusion. For example, it was shown that hyaluronic acid increases the permeability of the round window membrane, whereby allowing lidocaine to more rapidly diffuse into the inner ear and promote a larger effect (Selivanova, et al. Laryngo. Rhino. Otol. 2003, 82, 235-239, which is incorporated by reference herein in its entirety for all purposes). Further, for example, it was shown that transtympanic injection with histamine allowed for higher concentrations of dexamethasone in the perilymph of the inner ear than when administered without histamine (Chandrasekhar, S., Otol. Neurotol. 2001, 22, 18-23, which is incorporated by reference herein in its entirety for all purposes).

The membrane of the round or oval is the biological barrier to the inner ear space and represents the major obstacle for the local treatment of hearing impairment. The administered drug must overcome this membrane to reach the inner ear space. The drug can operatively (e.g., injection through the tympanic membrane) be placed locally to the round or oval membrane and can then penetrate through the round or oval membrane. Substances that penetrate the round or oval typically distribute in the perilymph and thus reach the hair cells and supporting cells.

In some embodiments, pharmaceutical formulations are adapted to administer the drug locally to the round or oval membrane. The pharmaceutical formulations may also contain a membrane penetration enhancer, which supports the passage of the agents mentioned herein through the round or oval membrane. Accordingly, liquid, gel or foam formulations may be used. It is also possible to apply the active ingredient orally or to employ a combination of delivery approaches.

Intratympanic (IT) delivery of drugs to the ear is increasingly used for both clinical and research purposes. Some groups have applied drugs in a sustained manner using microcatheters and microwicks, while the majority have applied them as single or as repeated IT injections (up to 8 injections over periods of up to 2 weeks).

Intratympanically applied drugs are thought to enter the fluids of the inner ear primarily by crossing the round window or oval window (RW) membrane. Calculations show that a major factor controlling both the amount of drug entering the ear and the distribution of drug along the length of the ear is the duration the drug remains in the middle ear space. Single, ‘one-shot’ applications or applications of aqueous solutions for few hours' duration result in steep drug gradients for the applied substance along the length of the cochlea and rapidly declining concentration in the basal turn of the cochlea as the drug subsequently becomes distributed throughout the ear.

Other injection approaches include by osmotic pump, or, by combination with implanted biomaterial, and by injection or infusion. In some embodiments, injection approaches include by osmotic pump by combination with injection or infusion. Biomaterials that can aid in controlling release kinetics and distribution of drug include hydrogel materials, degradable materials. In some embodiments, in situ gelling materials are used. Other materials include collagen or other natural materials including fibrin, gelatin, and decellularized tissues. Gelfoam may also be suitable.

Delivery may also be enhanced via alternate means including but not limited to agents added to the delivered composition such as penetration enhancers, or could be through devices via ultrasound, electroporation, or high speed jet.

Methods described herein can also be used for inner ear cell types that may be produced using a variety of methods know to those skilled in the art including those cell types described in PCT Application No. WO2012103012 A1.

With regard to human and veterinary treatment, the amount of a particular agent(s) that is administered may be dependent on a variety of factors, including the disorder being treated and the severity of the disorder; activity of the specific agent(s) employed; the age, body weight, general health, sex and diet of the patient; the time of administration, route of administration, and rate of excretion of the specific agent(s) employed; the duration of the treatment; drugs used in combination or coincidental with the specific agent(s) employed; the judgment of the prescribing physician or veterinarian; and like factors known in the medical and veterinary arts.

The agents described herein may be administered in a therapeutically effective amount to a subject in need of treatment. Administration of compounds described herein can be via any of suitable route of administration, particularly by intratympanically. Other routes include ingestion, or alternatively parenterally, for example intravenously, intra-arterially, intraperitoneally, intrathecally, intraventricularly, intraurethrally, intrasternally, intracranially, intramuscularly, intranasally, subcutaneously, sublingually, transdermally, or by inhalation or insulations, or topical by ear instillation for absorption through the skin of the ear canal and membranes of the eardrum. Such administration may be as a single or multiple oral dose, defined number of ear drops, or a bolus injection, multiple injections, or as a short- or long-duration infusion. Implantable devices (e.g., implantable infusion pumps) may also be employed for the periodic parenteral delivery over time of equivalent or varying dosages of the particular formulation. In some embodiments, the compounds are formulated as a sterile solution in water or another suitable solvent or mixture of solvents for parenteral administration. The solution may contain other substances such as salts, sugars (particularly glucose or mannitol), to make the solution isotonic with blood, buffering agents such as acetic, citric, and/or phosphoric acids and their sodium salts, and preservatives.

Compounds or compositions described herein can be administered by a number of methods sufficient to deliver the compound to the inner ear. Delivering a compound to the inner ear includes administering the compound to the middle ear, such that the compound may diffuse across the round or oval to the inner ear and administering a compound to the inner ear by direct injection through the round or oval membrane. Such methods include, but are not limited to auricular administration, by transtympanic wicks or catheters, or parenteral administration, for example, by intraauricular, transtympanic, or intracochlear injection.

In particular embodiments, the compounds, compositions and formulations of the disclosure are locally administered, meaning that they are not administered systemically.

In some embodiments, a syringe and needle apparatus is used to administer compounds or compositions to a subject using auricular administration. A suitably sized needle is used to pierce the tympanic membrane and a wick or catheter comprising the composition is inserted through the pierced tympanic membrane and into the middle ear of the subject. The device may be inserted such that it is in contact with the round or oval or immediately adjacent to the round or oval. Exemplary devices used for auricular administration include, but are not limited to, transtympanic wicks, transtympanic catheters, round or oval microcatheters (small catheters that deliver medicine to the round or oval), and Silverstein Microwicks™ (small tube with a “wick” through the tube to the round or oval, allowing regulation by subject or medical professional).

In another embodiment, a syringe and needle apparatus is used to administer compounds or compositions to a subject using transtympanic injection, injection behind the tympanic membrane into the middle and/or inner ear. The formulation may be administered directly onto the round or oval membrane via transtympanic injection or may be administered directly to the cochlea via intracochlear injection or directly to the vestibular organs via intravestibular injection.

In some embodiments, the delivery device is an apparatus designed for administration of compounds or compositions to the middle and/or inner ear. By way of example only: GYRUS Medical GmbH offers micro-otoscopes for visualization of and drug delivery to the round or oval niche; Arenberg has described a medical treatment device to deliver fluids to inner ear structures in U.S. Pat. Nos. 5,421,818; 5,474,529; and 5,476,446, each of which is incorporated by reference herein for such disclosure. U.S. patent application Ser. No. 08/874,208, which is incorporated herein by reference for such disclosure, describes a surgical method for implanting a fluid transfer conduit to deliver compositions to the inner ear. U.S. Patent Application Publication 2007/0167918, which is incorporated herein by reference for such disclosure, further describes a combined otic aspirator and medication dispenser for transtympanic fluid sampling and medicament application.

In some embodiments, composition provided herein is administered to a subject in need thereof once. In some embodiments, composition provided herein is administered to a subject in need thereof more than once. In some embodiments, a first administration of composition provided herein is followed by a second, third, fourth, or fifth administration of composition provided herein.

The number of times a compound is administered to an subject in need thereof depends on the discretion of a medical professional, the disorder, the severity of the disorder, and the subject's response to the formulation. In some embodiments, the compound disclosed herein is administered once to a subject in need thereof with a mild acute condition. In some embodiments, the compound disclosed herein is administered more than once to a subject in need thereof with a moderate or severe acute condition. In the case wherein the subject's condition does not improve, upon the doctor's discretion the compound may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the compound may administered continuously; alternatively, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once the subject's hearing and/or balance has improved, a maintenance dose can be administered, if necessary. Subsequently, the dosage or the frequency of administration, or both, is optionally reduced, as a function of the symptoms, to a level at which the improved disease, disorder or condition is retained. In some embodiments, subjects require intermittent treatment on a long-term basis upon any recurrence of symptoms.

In some embodiments, the pharmaceutical formulations may also contain an additional agent selected from a Notch activator, HDAC inhibitor, a BMP4 antagonist, Noggin (Inhibits BMP4), Sox2, Vitamin D (calcitriol), Vitamin B (nicotinomide), Vitamin A, Vitamin C (pVc). Lgr4, p38/MAPK inhibition, ROCK inhibition, and/or Alk4/7 inhibition. In some embodiments, the pharmaceutical formulations may also contain an epidermal growth factor (EGF), fibroblast growth factor (FGF), insulin-like growth factor (IGF), or a combination thereof.

Compositions with HDAC

In some embodiments, the pharmaceutical formulations may also contain HDAC.

In some embodiments, the pharmaceutical formulations containing HDAC can enhance the formation of Lgr5+ cells, control differentiation, control stemness, and replication or restore hearing and intestinal regeneration. In some embodiments, the pharmaceutical compositions may also contain an HDAC inhibitor.

In some embodiments, the pharmaceutical composition comprises a compound of the present disclosure, or a pharmaceutically acceptable salt or tautomer thereof, and an HDAC inhibitor. In some embodiments, the pharmaceutical composition can further comprise an additional pharmaceutically active agent. In some embodiments, the pharmaceutical composition can further comprise a poloxamer.

In some embodiments, the HDAC inhibitor is Valproic acid or a prodrug, ester, salt form, or amide thereof. In some embodiments, the HDAC inhibitor is Valproic acid or a pharmaceutically acceptable salt or tautomer thereof. In some embodiments, the HDAC inhibitor is sodium Valproate.

In some embodiments, the HDAC inhibitor is a carboxylic acid containing compound. In some embodiments, the carboxylic acid containing compound is C6-C20 carboxylic acid, wherein the carboxylic acid comprises alkyl, alkenyl, or alkynyl.

In some embodiments, the carboxylic acid containing compound is a substituted or unsubstituted C5-C20 straight, branched, or cyclic chain alkyl-CO₂H, substituted or unsubstituted C5-C20 straight, branched, or cyclic chain alkenyl-CO₂H and substituted or unsubstituted C5-C20 straight, branched, or cyclic chain alkynyl-CO₂H. In some embodiments, the carboxylic acid containing compound is a substituted C5-C20 straight or branched chain alkyl-CO₂H.

In some embodiments, the carboxylic acid containing compound is a substituted C5-C20 straight or branched chain alkyl-CO₂H, wherein the substituent is —NH₂. In some embodiments, the carboxylic acid containing compound is an amino substituted 2-propylpentanoic acid. In some embodiments, the amino substituted 2-propylpentanoic acid is selected from the group consisting of 5-amino-2-propylpentanoic acid, 4-amino-2-propylpentanoic acid, 3-amino-2-propylpentanoic acid, and 2-amino-2-propylpentanoic acid.

In some embodiments, the carboxylic acid containing compound is an unsubstituted C5-C20 straight or branched chain alkyl-CO₂H. In some embodiments, the carboxylic acid containing compound is an unsubstituted C6-C9 branched straight chain alkyl-CO₂H. In some embodiments, the carboxylic acid containing compound is an unsubstituted C₈-C₉ branched straight chain alkyl-CO₂H. In some embodiments, the carboxylic acid containing compound is an unsubstituted C8 branched straight chain alkyl-CO₂H.

In some embodiments, the carboxylic acid containing compound is Valproic acid.

In some embodiments, the carboxylic acid containing compound is in the form of a prodrug of an unsubstituted Cs branched straight chain alkyl-CO₂H wherein the prodrug is in the form of an amide or ester. In some embodiments, the amide of unsubstituted Cs branched straight chain alkyl-CO₂H is the condensation product with an amino acid. In some embodiments, the amide of Valproic acid is selected from the group consisting of

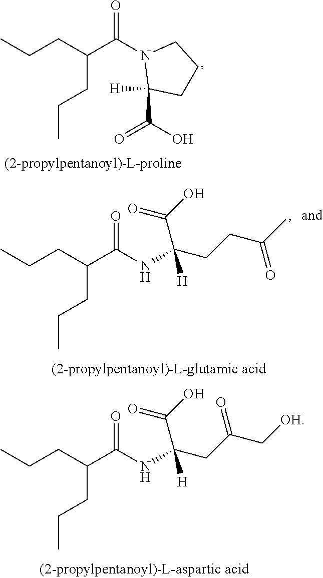

In some embodiments, the HDAC inhibitor is any one of the inhibitors listed in Table 1.

TABLE 1
HD AC Inhibitors

| Class | Agent | CAS Number |
|---|---|---|
| Aliphatic Acid | Valproic Acid | 99-66-1 |
| Aliphatic Acid | Phenyl butyrate | 1821-12-1 |
| Aliphatic Acid | Butyrate | 107-92-6 |
| Aliphatic Acid | 2-(prop-2-yn-1-yl) octanoic acid | 96017-59-3 |
| Aliphatic Acid | (S)-2-(prop-2-yn-1-yl) octanoic acid | 185463-37-0 |
| Aliphatic Acid | ® -2-(prop-2-yn-1-yl) octanoic acid | 185463-38-1 |
| Aliphatic Acid | 2-(prop-2-yn-1-yl) heptanoic acid | 176638-49-6 |
| Aliphatic Acid | (S)-2-(prop-2-yn-1-yl) | 185463-37-0 |
| | heptanoic acid | |
| Aliphatic Ac ® (R)-2-(prop-2-yn-1-yl) heptanoic acid | | 185463-38-1 |
| Aliphatic Acid | 2-fluoro-2-propyl Pentanoic acid | 197779-85-4 |
| Aliphatic Acid Ester | AN-9 | 122110-53-6 |
| Amine | 932718-22-4 | 932718-22-4 |
| Benzamide | Entinostat (MS-275) | 209783-80-2 |
| Benzamide | Mocetinostat (MGCD0103) | 726169-73-9 |
| Benzamide | Tacedinaline | 112522-64-2 |
| Benzamide | BML-210 | 537034-17-6 |
| Benzamide | NKL 22 | 537034-15-4 |
| Benzamide | RGFP109 | 1215493-56-3 |
| Benzamide | RGFP136 | 1215493-97-2 |
| Benzamide | RGFP966 | 1357389-11-7 |
| Benzamide | 4SC-202 | 1186222-89-8 |
| Benzamide | HDAC Inhibitor IV | 537034-15-4 |
| Benzamide | Chidamide | 743438-44-0 |
| Benzamide | TC-H 106, HDAC Inhibitor VII | 937039-45-7 |
| Cyclic peptide | Romidepsin | 128517-07-7 |
| Cyclic peptide | Trapoxin A | 133155-89-2 |
| Cyclic peptide | HC Toxin | 83209-65-8 |
| Cyclic peptide | Apicidin | 183506-66-3 |
| Cyclic Peptide | Thailandepsin A | 1269219-30-8 |
| Cyclic peptide | Dihydrochlamydocin | 52574-64-8 |
| Epoxide | (—)-Depudecin | 139508-73-9 |
| Epoxide | Parthenolide | 20554-84-1 |
| Hydroxamate | Trichostatin A (TSA) | |
| Hydroxamate | Trichostatin A (TSA) | 58880-19-6 |
| Hydroxamate | SAHA (Zolinza, vorinostat) | 149647-78-9 |
| Hydroxamate | 4-iodo-SAHA | 1219807-87-0 |
| Hydroxamate | SBHA | 38937-66-5 |
| Hydroxamate | CBHA | 174664-65-4 |
| Hydroxamate | LAQ-824 | 591207-53-3 |
| Hydroxamate | PDX-101 (belinostat) | 866323-14-0 |
| Hydroxamate | LBH-589 (panobinostat) | 404950-80-7 |
| Hydroxamate | ITF2357 (Givinostat) | 497833-27-9 |
| Hydroxamate | PCI-34051 | 950762-95-5 |
| Hydroxamate | PCI-24781 (Abexinostat) | 783355-60-2 |
| Hydroxamate | Tubastatin A | 1252003-15-8 |
| Hydroxamate | CUDC-101 | 1012054-59-9 |
| Hydroxamate | Oxamflatin | 151720-43-3 |
| Hydroxamate | ITF2357 | 497833-27-9 |
| Hydroxamate | Bufexamac | 2438-72-4 |
| Hydroxamate | APHA Compound 8 | 676599-90-9 |
| Hydroxamate | HDAC Inhibitor XXIV | 854779-95-6 |
| Hydroxamate | Tubacin | 537049-40-4 |
| Hydroxamate | Butyrylhydroxamic acid | 4312-91-8 |
| Hydroxamate | MC 1568 | 852475-26-4 |
| Hydroxamate | SB939 (Pracinostat) | 929016-96-6 |
| Hydroxamate | 4SC-201 (Resminostat) | 864814-88-0 |
| Hydroxamate | Tefinostat (CHR-2845) | 914382-60-8 |
| Hydroxamate | CHR-3996 | 1256448-47-1 |
| Hydroxamate | NSC 57457 | 6953-61-3 |
| Hydroxamate | CG200745 | 936221-33-9 |
| Hydroxamate | ACY1215 | 1316214-52-4 |
| Hydroxamate | Nexturastat A | 1403783-31-2 |
| Hydroxamate | Droxinostat | 99873-43-5 |
| Hydroxamate | Scriptaid | 287383-59-9 |
| Hydroxamate | BRD9757 | 1423058-85-8 |
| Hydroxamate | HPOB | 1429651-50-2 |
| Hydroxamate | CAY10603 | 1045792-66-2 |
| Hydroxamate | HDAC6 Inhibitor III | 1450618-49-1 |
| Hydroxamate | M 344 | 251456-60-7 |
| Hydroxamate | 4-(dimethylamino)-N-[6- | 193551-00-7 |
| | (hydroxyamino)-6-oxohexyl]- | |
| | benzamide | |
| Hydroxamate | (S)-HDAC-42 | 935881-37-1 |
| Hydroxamate | HNHA | 926908-04-5 |
| Hydroxamate | Pyroxamide | 382180-17-8 |
| Hydroxamate | HDAC Inhibitor VI | 926908-04-5 |
| Hydroxamate | HDAC Inhibitor II | 174664-65-4 |
| Hydroxamate | LMK235 | 1418033-25-6 |
| Hydroxamate | HDAC-IN-1 | 1239610-44-6 |
| Hydroxamate | VAHA | 106132-78-9 |
| K-tone-CF3 | Compound 6e | 946500-31-8 |
| K-tone-CF3 | Compound 6H | 946500-39-6 |
| K-tone-CF3 | Compound 27 | 946499-86-1 |
| Ketone | Compound 43 | 891259-76-0 |
| K-tone-a-ketoamides | 436150-82-2 | 436150-82-2 |
| Polyketide | Ratjadone A | 163564-92-9 |
| Silylalcohol | 1587636-32-5 | 1587636-32-5 |
| Sulphonyl Urea | 960130-17-0 | 960130-17-0 |
| Sulphonamide | 1587636-33-6 | 1587636-33-6 |
| Sulphonamide | 329967-25-1 | 329967-25-1 |
| Thiol | 1428536-05-3 | 1428536-05-3 |
| Thiol | 908860-21-9 | 908860-21-9 |
| Thiol | 828920-13-4 | 828920-13-4 |
| Thiol | 1368806-68-1 | 1368806-68-1 |
| Thiol | 827036-76-0 | 827036-76-0 |
| Thioester | TCS HDAC6 20b | 956154-63-5 |
| Thioester | PTACH | 848354-66-5 |
| Thioester | KD 5170 | 940943-37-3 |
| Thioester | HD AC Inhibitor XXII | 848354-66-5 |
| Thioketone | SIRT1/2 Inhibitor VH | 143034-06-4 |
| Tropones | 46189-88-2 | 46189-88-2 |
| Tropones | 1411673-95-4 | 1411673-95-4 |
| Non classical | TMP269 | 1314890-29-3 |
| Non classical | Tasquinimod | 254964-60-8 |

Classes of HDAC inhibitors for use in various embodiments of the compositions and methods disclosed herein include but are not limited to those listed in Column A of Table 1. Specific HDAC inhibitors for use in various embodiments of the compositions and methods disclosed herein include but are not limited to those listed in Column B of Table 1. All agents listed in Table 1 column B are understood to include derivatives or pharmaceutically acceptable salts thereof. All classes listed in Table 1 column A are understood to include both agents comprising that class and derivatives or pharmaceutically acceptable salts thereof.

In some embodiments, the amount of the carboxylic acid containing compound is between least 2 wt % (weight carboxylic acid containing compound/weight pharmaceutical composition) and 20 wt %. In some embodiments, the composition comprises at least 4 wt % carboxylic acid. In some embodiments, the composition comprises at least 8 wt % carboxylic acid. In some embodiments, the composition comprises at least 12 wt % carboxylic acid. In some embodiments, the composition comprises at least 16 wt % carboxylic acid. In some embodiments, the composition comprises at least 20 wt % carboxylic acid.

Compositions with Tranykypromine

In some embodiments, the pharmaceutical composition comprises a compound of the present disclosure, or a pharmaceutically acceptable salt or tautomer thereof, and at least one additional pharmaceutically active agent. In some embodiments, the pharmaceutical composition comprises a compound of the present disclosure, or a pharmaceutically acceptable salt or tautomer thereof, and tranylcypromine or a pharmaceutically acceptable salt or tautomer thereof. In some embodiments, the pharmaceutical composition can further comprise an additional pharmaceutically active agent. In some embodiments, the pharmaceutical composition can further comprise a poloxamer.

In some embodiments, the pharmaceutical compositions may also contain tranylcypromine or a pharmaceutically acceptable salt or tautomer thereof. In some embodiments, the pharmaceutical compositions containing tranylcypromine, or a pharmaceutically acceptable salt or tautomer thereof, can enhance the formation of Lgr5+ cells, control differentiation, control sternness, and replication or restore hearing and intestinal regeneration. In some embodiments, the pharmaceutical compositions may also contain an HDAC inhibitor. In some embodiments, the HDAC inhibitor is Valproic acid or a prodrug, ester, salt form, or amide thereof. In some embodiments, the HDAC inhibitor is Valproic acid or a pharmaceutically acceptable salt or tautomer thereof. In some embodiments, the HDAC inhibitor is sodium Valproate.

Compositions with LSD Inhibitors

In some embodiments, the pharmaceutical compositions may also contain an LSD-1 inhibitor. In some embodiments, the pharmaceutical formulations containing an LSD-1 can enhance the formation of Lgr5+ cells, control differentiation, control sternness, and replication or restore hearing and intestinal regeneration. In some embodiments, the pharmaceutical compositions may also contain an HDAC inhibitor.

In some embodiments, the LSD-1 inhibitor is any one of the inhibitors listed in Table 2.

TABLE 2
LSD-1 inhibitors

| Agent | CAS | Agent | CAS |
|---|---|---|---|
| GSK-2879552 | 1401966-69-5 | Pargyline | 555-57-7 |
| GSK-LSD-1 | 1431368-48-7 | Peptide | 945548-35-6 |
| Phenelzine sulfate | 51-71-8 | Bizine | 1591932-50-1 |
| TCP | 155-09-9 | Compound 5a | 1990536-90-7 |
| (Tranylcypromine) | | | |
| | | Compound 5n | 1990537-03-5 |
| CC-90011 | 2179319-65-2 | SP-2509 (HCI- | 1423715-09-6 |
| | | 2509) | |
| GCG-11047 | 308145-19-9 | LSD-1-IN-32 | 2137044-49-4 |
| (PG-11047) | | | |
| IMG-7289 | 2229826-41-7 | LSD-1-IN-11p | 2101951-67-9 |
| INCB059872 | 1802909-49-4 | Resveratrol | 501-36-0 |
| ORY-1001 | 1431326-61-2 | Hydroxylamine | 2035912-55-9 |
| (RG6016, | | | |
| RO7051790, | | | |
| Iadademstat) | | | |
| ORY-2001 | 1357362-02-7 | Compound 8c | 2170023-28-4 |
| (Vafidemstat) | | | |
| Osimertinib | 1421373-65-0 | CBB-1007 | 1379573-92-8 |
| (AZD9291) | | | |
| SP-2577 | 1423715-37-0 | Namoline | 342795-11-3 |
| (Seclidemstat) | | | |
| | 1821307-10-1 | GSK354 | 1841508-96-0 |
| TCP Trans Chiral | 3721-28-6 | GSK-690 | 2101305-84-2 |
| TCP Trans Chiral | 3721-26-4 | E11 | 1239589-91-3 |
| TCP Cis | 13531-35-6 | MC2694 | 1435055-66-5 |
| TCP Cis Chiral | 69684-88-4 | Alpha-mangostin | 11/1/6147 |
| TCP Cis Chiral | 69684-89-5 | Compound 12A | 1923750-07-5 |
| | | Compound 4 | 126118-57-8 |
| RN-1 | 1781835-13-9 | Compound 10d | 2226997-31-3 |
| Compound 1 | 1221595-26-1 | Compound 90 | 1884266-15-2 |
| Compound 45 | 1667721-01-8 | Compound 46 | 1884266-36-7 |
| RN-7 | 1352345-82-4 | Compound 49 | 1884266-49-2 |
| Compound 5A | 1613476-09-7 | Compound 50 | 1884266-48-1 |
| Compound 2 | 1235863-51-0 | Polymyxin B | 1404-26-8 |
| Compound 43 | 1784703-61-2 | Polymyxin E | 1066-17-7 |
| Compound 12f | 1802319-25-0 | Baicalin | 21967-41-9 |
| T-3775440 | 1422620-34-5 | Compound 16Q | 1612870-90-2 |
| OG-L002 | 1357299-45-6 | LSD-1 inhibitor 24 | 1853269-07-4 |
| S2101 | 1239262-36-2 | geranylgeranoic | 35750-48-2 |
| | | acid | |
| NCL-1 | 1196119-03-5 | Geranylgeraniol | 24034-73-9 |
| Compound 9A | 2095849-74-2 | Thiocarbamate | 1430852-56-4 |
| Compound 19l | 2173543-81-0 | Thiourea | 1637373-61-5 |
| NCD-25 | 1456972-46-5 | Thiourea | 2035417-23-1 |
| NCD-38 | 2078047-42-2 | Thienopyrrole | 1206028-57-0 |
| | | Thienopyrrole | 1884266-15-2 |
| Compound 14A | 2247939-53-1 | Thienopyrrole | 1884266-48-1 |
| Compound 15A | 2247939-55-3 | 4SC-202 | 910462-43-0 |
| Compound 15B | 2247939-56-4 | ORY-3001 | 2179325-30-3 |
| Compound 4 | 2226461-60-3 | JL1037 | |
| | | FLI-06 | 313967-18-9 |

All agents listed in Table 2 are understood to include derivatives or pharmaceutically acceptable salts thereof.

In some embodiments, the LSD-1 inhibitor is GSK-2879552, GSK-LSD-1, Osimertinib (AZD9291), Phenelzine sulfate, Tranylcypromine (TCP), ORY-1001, Seclidemstat (SP-2577), Vafidemstat (ORY-2001), CC-90011, IMG-7289 or, INCB059872. In some embodiments, the LSD-1 inhibitor is GSK-2879552, GSK-LSD-1, Phenelzine sulfate or Tranylcypromine (TCP). In some embodiments, the LSD-1 inhibitor is GSK-2879552.

Compositions with Additional Pharmaceutically Active Agents

In some embodiments, the pharmaceutical composition comprises a compound of the present disclosure, or a pharmaceutically acceptable salt or tautomer thereof, and at least one additional pharmaceutically active agent. In some embodiments, the pharmaceutical composition comprises a compound of the present disclosure, or a pharmaceutically acceptable salt or tautomer thereof, and CHIR99021, or a pharmaceutically acceptable salt or tautomer thereof. In some embodiments, the pharmaceutical composition comprises a compound of the present disclosure, or a pharmaceutically acceptable salt or tautomer thereof, and LY2090314, or a pharmaceutically acceptable salt or tautomer thereof.

In some embodiments, the pharmaceutical composition further comprises tranylcypromine or a pharmaceutically acceptable salt or tautomer thereof. In some embodiments, the pharmaceutical composition further comprises an HDAC inhibitor. In some embodiments, the pharmaceutical composition further comprises an LSD-1 inhibitor. In some embodiments, the pharmaceutical composition further comprises an additional pharmaceutically active agent. In some embodiments, the pharmaceutical composition further comprises a poloxamer.

In some embodiments, the pharmaceutical composition further comprises growth factor. In some embodiments, the growth factor is a protein. In some embodiments, the growth factor is a hormone. In some embodiments, the growth factor is epidermal growth factor (EGF), basic fibroblast growth factor (bFGF), insulin-like growth factor 1 (IGR1), or a combination thereof. In some embodiments, the growth factor is EGF. In some embodiments, the growth factor is bFGF. In some embodiments, the growth factor is IGR1. In some embodiments, the growth factor is a combination of EGF and bFGF. In some embodiments, the growth factor is a combination of EGF and IGR1. In some embodiments, the growth factor is a combination of bFGF and IGR1. In some embodiments, the growth factor is a combination of EGF, bFGF, and IGR1.

Compositions with Poloxamers

In some aspects, the present disclosure provides a pharmaceutical composition comprising: a) a compound of the present disclosure and b) a poloxamer. In some aspects, the present disclosure provides a pharmaceutical composition comprising: a) a compound or mixture of compounds, or pharmaceutically acceptable salts or tautomers thereof, and b) a poloxamer.

In some aspects, the present disclosure the pharmaceutical compositions are lyophilized. comprising one or more agents described herein and a gelling agent.

In some aspects, the present disclosure provides a lyophilized pharmaceutical composition, comprising one or more agents described herein and a gelling agent (e.g., a poloxamer).

In some embodiments, the lyophilized pharmaceutical composition is in the form of a lyophilized cake.

In some embodiments, the lyophilized pharmaceutical composition has a higher stability to oxygen and/or light as compared to a comparable pharmaceutical composition comprising one or more solvents.

In some embodiments, the present disclosure provides a reconstituted solution of the lyophilized pharmaceutical compositions.

As used herein, the term “gelling agent” refers to an agent capable of imparting a gel-like or thickening quality to the pharmaceutical composition or reconstituted solution of the present disclosure upon being subjected to a gelling condition (e.g., a particular temperature or temperature range, the presence of an ion, a pH value or range, or a concentration of gelling agent that causes the gelling agent to undergoing a change or transition from low viscosity to high viscosity, or the reverse). In some embodiments, the gelling condition is a particular temperature (e.g., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.). In some embodiments, the gelling condition is a particular temperature range (e.g., about 26° C. or higher, about 27° C. or higher, about 28° C. or higher, about 29° C. or higher, about 30° C. or higher, about 31° C. or higher, about 32° C. or higher, about 33° C. or higher, about 34° C. or higher, about 35° C. or higher, about 36° C. or higher, about 37° C. or higher, about 38° C. or higher, about 39° C. or higher, or about 40° C. or higher). In some embodiments, the gelling agent provides a viscosity of between about 1,000 and 10,000,000 centipoise, between about 5,000 and 5,000,000 centipoise, or between about 100,000 and 4,000,000 centipoise, to the pharmaceutical composition or reconstituted solution of the present disclosure. In some embodiments, the gelling agent provides a viscosity of between about 50,000 and 2,000,000 centipoise to the pharmaceutical composition or reconstituted solution of the present disclosure.

In some embodiments, prior to gelling (e.g., at ambient temperature (e.g., between about 20° C. and about 26° C.)), the gelling agent provides a viscosity of less than about 100,000 centipoise, less than about 50,000 centipoise, 20,000 centipoise, less than about 10,000 centipoise, less than about 8,000 centipoise, less than about 7,000 centipoise, less than about 6,000 centipoise, less than about 5,000 centipoise, less than about 4,000 centipoise, less than about 3,000 centipoise, less than about 2,000 centipoise, or less than about 1,000 centipoise to the pharmaceutical composition or reconstituted solution of the present disclosure.

In some embodiments, upon gelling (e.g., at the temperature of a human body (e.g., between about 35° C. to about 39° C., between about 36° C. to about 38° C., or at about 37° C.)), the gelling agent provides a viscosity of greater than about 1,000 centipoise, greater than about 5,000 centipoise, greater than about 10,000 centipoise, greater than about 20,000 centipoise, greater than about 50,000 centipoise, greater than about 60,000 centipoise, greater than about 70,000 centipoise, greater than about 80,000 centipoise, greater than about 90,000 centipoise, or greater than about 100,000 centipoise.

In some embodiments, upon gelling (e.g., at the temperature of a human body (e.g., between about 36° C. to about 39° C., or at about 37° C.)), the viscosity of the pharmaceutical composition or reconstituted solution of the present disclosure, as measured in units of centipoise, being about 2 fold or greater, about 5 fold or greater, about 10 fold or greater, about 20 fold or greater, about 50 fold or greater, about 60 fold or greater, about 7 fold or greater, about 80 fold or greater, about 90 fold or greater, about 100 fold or greater as compared to the viscosity of the pharmaceutical composition or reconstituted solution prior to gelling (e.g., at ambient temperature (e.g., at about 25° C.)).

It is understood that the gelling condition (e.g., gelling temperature) of the pharmaceutical composition or reconstituted solution of the present disclosure may be measured with a variety of techniques in the art. In some embodiment, the gelling temperature is determined using a commercially available rheomoeter having a parallel plate geometry (e.g., with plate distance ranging from 0.5 mm to 1.0 mm). In some embodiments, the analysis is performed over a continuous temperature range (e.g., 15° C. to 40° C.) at a constant rate (e.g., 2 to 3° C./min) and a deformation frequency of 0.74 Hz to 1 Hz. The geleation temperature is determined at the temperature whereby the shear storage modulus (G′) and the shear loss modulus (G″) are equal.

In some embodiments, the gelling agent comprises acacia, alginic acid, bentonite, poly(acrylic acid) (Carbomer), carboxymethyl cellulose, ethylcellulose, gelatin, hydroxyethyl cellulose, hydroxypropyl cellulose, magnesium aluminum silicate (Veegum), methylcellulose, poloxamer, hyaluronic acid sodium, polylacticglycolic acid sodium, chitosan, polyvinyl alcohol, sodium alginate, tragacanth, xanthan gum, or any combination thereof. In some embodiment, the gelling agent comprises poloxamer.

In some embodiments, the gelling agent is a thermoreversible gelling agent.

As used herein, the term “thermoreversible” refers to a capability of being reversible by the application of heat. The “thermoreversible gelling agent” refers to an agent capable of reversibly imparting a gel-like or thickening quality to the pharmaceutical composition or reconstituted solution of the present disclosure upon application of heat.

In some embodiments, the thermoreversible gelling agent comprises a poloxamer.

It is understood that the gelling agent (e.g., the thermoreversible gelling agent) may also be a bulking agent of the pharmaceutical composition or reconstituted solution of the present disclosure. In some embodiments, a poloxamer (e.g., poloxamer 407) is the gelling agent and/or the bulking agent of the pharmaceutical composition or reconstituted solution of the present disclosure. Poloxomers are a general class of commercially available and pharmaceutically acceptable triblock copolymers of polyethylene oxide-polypropylene oxide-polyethylene oxide which exhibit relatively low viscosity at low temperatures (e.g., room temperature or below) but much high viscosities at elevated temperatures (e.g., body temperatures of approximately 37° C.) whereby compositions containing such thermoreversible gelling agents effectively solidify in place. Other thermoreversible gelling agents such as polyethylene oxide-polylactic acid-polyethylene oxide polymers are also suitable in various embodiments of the present invention.

In some embodiments, the poloxamer (e.g., poloxamer 407) is the gelling agent and the bulking agent of the pharmaceutical composition or reconstituted solution of the present disclosure. In some embodiments, the presence of the poloxamer (e.g., poloxamer 407) in the pharmaceutical composition (e.g., the lyophilized pharmaceutical composition) alleviates the need for any other excipient (e.g., additional bulking agent). Such alleviation may provide one or more advantages to the pharmaceutical composition (e.g., enhanced stability and/or reduced reconstitution time).

In some embodiments, the poloxamer the poloxamer is a thermoreversible gel. In some embodiments, the poloxamer is a gel at about body temperature (e.g., 37° C.). In some embodiments, the poloxamer is an immobile gel at about body temperature.

In some embodiments, the poloxamer is selected from the group consisting of Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407.

In some embodiments, the poloxamer is Poloxamer 188 or Poloxamer 407.

In some embodiments, the poloxamer is Poloxamer 407.

In some embodiments, the poloxamer comprises a polyethylene oxide-polypropylene oxide-polyethylene oxide triblock copolymer. In some embodiments, poloxamer comprises at least 50% polyethylene oxide by molecular mass. In some embodiments, the poloxamer comprises 60-80% polyethylene oxide by molecular mass. In some embodiments, the poloxamer comprises

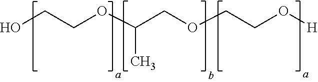

where a is 80-120 and b is 50-70.

In some embodiments, the poloxamer is a purified poloxamer (e.g., purified Poloxamer 407). General guidelines on purifying polymers are available, e.g., in U.S. Pat. No. 6,977,045, Fakhari et al. (Heliyon 3:e00390 (2017)), and PCT Application Publication No. WO/2017/108457, each of which is incorporated herein by reference. The liquid-liquid extraction procedure involves the fractionation of the poloxamer (e.g., Poloxamer 407) between two aqueous phases containing with different salt concentration. In some embodiments, one or more impurities preferentially partition into the aqueous phase with high salt concentration, and the purified poloxamer (e.g., Poloxamer 407) remains in the aqueous phase with low salt concentration. The size exclusion chromatography provides separation based on hydrodynamic radius. The fractions containing purified poloxamer (e.g., Poloxamer 407) with the desired molecular weight range are collected.

In some embodiments, the poloxamer has a number average molecular weight of about 10,800 to about 11,200 Da. In some embodiments, the poloxamer has a weight average molecular weight of about 11,500 to about 11,700 Da. In some embodiments, the poloxamer ranges from about 7,250 to about 16,600 Da. In some embodiments, at least 87% by weight of the poloxamer has an average molecular weight of greater than about 7,250 Da. In some embodiments, less than 15% by weight of the poloxamer has an average molecular weight less about 7,250 Da. The number average molecular weight/weight average molecular weight is determined by a six point molecular weight calibration curve, generated using polyethylene glycol standards ranging from 1,450 Da to 35,000 Da. The PEG standards are Polyethylene glycol EasiVials (2 mL), Agilent, part number PL 2070-0201. In some embodiments, the average molecular weight is the weight average molecular weight, characterized by liquid-liquid extraction or size exclusion chromatography. In some embodiments, the average molecular weight is the number average molecular weight, characterized by liquid-liquid extraction or size exclusion chromatography.

In some embodiments, the purified poloxamer (e.g., purified Poloxamer 407) has an average molecular weight of about 9 kDa or greater, about 9.2 kDa or greater, about 9.4 kDa or greater, about 9.6 kDa or greater, about 9.8 kDa or greater, about 10 kDa or greater, about 10.2 kDa or greater, about 10.4 kDa or greater, about 10.6 kDa or greater, about 10.8 kDa or greater, about 11 kDa or greater, about 11.2 kDa or greater, about 11.4 kDa or greater, about 11.6 kDa or greater, about 11.8 kDa or greater, about 12 kDa or greater, or about 12.1 kDa or greater.

In some embodiments, the purified poloxamer (e.g., purified Poloxamer 407) has a reduced level of polymer chains with molecular weight below 9 kDa as compared to the unpurified poloxamer (e.g., unpurified Poloxamer 407).

In some embodiments, the purified poloxamer (e.g., purified Poloxamer 407) has about 99% or less, about 98% or less, about 95% or less, about 90% or less, about 80% or less, about 70% or less, about 60% or less, about 50% or less, about 40% or less, about 30% or less, about 20% or less, or about 10% or less of polymer chains with molecular weight below 9 kDa as compared to the unpurified poloxamer (e.g., unpurified Poloxamer 407).

In some embodiments, the purified poloxamer (e.g., purified Poloxamer 407) is prepared by liquid-liquid extraction or size exclusion chromatography.

In some embodiments, the purified poloxamer (e.g., purified Poloxamer 407) is characterized by liquid-liquid extraction or size exclusion chromatography.

In some embodiments, about 10% or more, about 20% or more, about 30% or more, about 40% or more, about 50% or more, about 60% or more, about 70% or more, about 80% or more, about 90% or more, about 95% or more, about 98% or more, or about 99% or more of the one or more impurities having molecular weights below 9 kDa are removed from the poloxamer (e.g., Poloxamer 407) during the purification.

In some embodiments, about 10% or more, about 20% or more, about 30% or more, about 40% or more, about 50% or more, about 60% or more, about 70% or more, about 80% or more, about 90% or more, about 95% or more, about 98% or more, or about 99% or more of the one or more diblock copolymers (e.g., PEO-PPO), single block polymers (e.g., PEO), and/or aldehydes are removed from the poloxamer (e.g., Poloxamer 407) during the purification.

In some embodiments, the pharmaceutical composition, pharmaceutical composition, the lyophilized pharmaceutical composition or reconstituted solution of the present disclosure comprises a buffering agent. The buffer controls the pH of the reconstituted solution to a range of from about 4 to about 13, from about 5 to about 12, from about 6 to about 11, from about 6.5 to about 10.5, or from about 7 to about 10.

Examples of the buffering agent include, but are not limited to, citrate buffering agents, acetate buffering agents, phosphate buffering agents, ammolnium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. Lubricating agents may be selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

In some embodiments, the buffering agent comprises phosphate buffered saline, TRIS, tris acetate, tris HCl-65, sodium citrate, histidine, arginine, sodium phosphate, tris base-65, hydroxyethyl starch, or any combination thereof.

In some embodiments, the pharmaceutical composition, pharmaceutical composition, the lyophilized pharmaceutical composition or reconstituted solution of the present disclosure comprises a bulking agent.

In some embodiments, the bulking agent comprises poloxamer (e.g., poloxamer 407), mannitol, sucrose, maltose, trehalose, dextrose, sorbitol, glucose, raffinose, glycine, histidine, polyvinylpyrrolidone (e.g., polyvinylpyrrolidone K12 or polyvinylpyrrolidone K17), lactose, or any combination thereof.

In some embodiments, the pharmaceutical composition, pharmaceutical composition, the lyophilized pharmaceutical composition or reconstituted solution of the present disclosure comprises a stabilizing agent.

In some embodiments, the stabilizing agent comprises a cryoprotectant. In some embodiments, the cryoprotectant is a polyol (e.g., a diol or a triol such as propylene glycol (i.e., 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a polymer (e.g., polyethylene glycol 200 (PEG 200), PEG 400, PEG 600, PEG 1000, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g., polyvinylpyrrolidone K 15), pentaerythritol propoxylate, or polypropylene glycol P 400), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g., lithium acetate, lithium chloride, lithium formate, lithium nitrate, lithium sulfate, magnesium acetate, sodium chloride, sodium formate, sodium malonate, sodium nitrate, sodium sulfate, or any hydrate thereof) or any combination thereof.

In some embodiments, the stabilizing agent comprises a salt. In some embodiment, the salt is selected from the group consisting of lithium salts (e.g., lithium acetate, lithium chloride, lithium formate, lithium nitrate, lithium sulfate, or any hydrate thereof), magnesium salts (e.g., magnesium acetate or a hydrate thereof), and sodium salts (e.g., sodium chloride, sodium formate, sodium malonate, sodium nitrate, sodium sulfate, or any hydrate thereof). For another example, the formulation comprises one or more sodium salts. For yet another example, the formulation comprises sodium chloride.

In some embodiment, the stabilizing agent comprises a surfactant. In some embodiments, the surfactant comprises one or more anionic surfactants (e.g., 2-acrylamido-2-methylpropane sulfonic acid, ammolnium lauryl sulfate, ammolnium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium dodecylbenzenesulfonate, sodium laurate, sodium laureth sulfate, sodium lauroyl sarcosinate, sodium myreth sulfate, sodium nonanoyloxybenzenesulfonate, sodium pareth sulfate, sodium stearate, or sulfolipid), one or more cationic surfactants (e.g., behentrimonium chloride, benzalkonium chloride, benzethonium chloride, benzododecinium bromide, bronidox, carbethopendecinium bromide, cetalkonium chloride, cetrimonium bromide, cetrimonium chloride, cetylpyridinium chloride, didecyldimethylammolnium chloride, dimethyldioctadecylammolnium bromide, dimethyldioctadecylammolnium chloride, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammolnium hydroxide, or thonzonium bromide), one or more zwitterionic surfactants (e.g., cocamidopropyl betaine, cocamidopropyl hydroxysultaine, dipalmitoylphosphatidylcholine, egg lecithin, hydroxysultaine, lecithin, myristamine oxide, peptitergents, or sodium lauroamphoacetate), and/or one or more non-ionic surfactants (e.g., alkyl polyglycoside, cetomacrogol 1000, cetostearyl alcohol, cetyl alcohol, cocamide dea, cocamide mea, decyl glucoside, decyl polyglucose, glycerol monostearate, igepal ca-630, isoceteth-20, lauryl glucoside, maltosides, monolaurin, mycosubtilin, narrow-range ethoxylate, nonidet p-40, nonoxynol-9, nonoxynols, np-40, octaethylene glycol monododecyl ether, n-octyl beta-d-thioglucopyranoside, octyl glucoside, oleyl alcohol, peg-10 sunflower glycerides, pentaethylene glycol monododecyl ether, polidocanol, α-tocopheryl polyethylene glycol succinate (TPGS), poloxamer (e.g., poloxamer 407), polyethoxylated tallow amine, polyglycerol polyricinoleate, polysorbate (e.g., polysorbate 20, polysorbate 40, polysorbate 60, or polysorbate 80), sorbitan, sorbitan monolaurate, sorbitan monostearate, sorbitan tristearate, stearyl alcohol, surfactin, triton x-100).

In some embodiments, the pharmaceutical composition, pharmaceutical composition, the lyophilized pharmaceutical composition or reconstituted solution of the present disclosure comprises a tonicity-adjusting agent.

In some embodiments, the tonicity-adjusting agent comprises NaCl, dextrose, dextran, ficoll, gelatin, mannitol, sucrose, glycine, glycerol, or any combination thereof.

In some embodiments, the pharmaceutical composition or reconstituted solution of the present disclosure comprises a soothing agent. In some embodiments, the soothing agent comprises lidocaine

In addition to these components, the pharmaceutical composition, pharmaceutical composition, the lyophilized pharmaceutical composition or reconstituted solution of the present disclosure includes any substance useful in pharmaceutical compositions.

In some embodiments, the pharmaceutical composition, pharmaceutical composition, the lyophilized pharmaceutical composition or reconstituted solution of the present disclosure includes one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21st Edition, A. R Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammolnium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and or combinations thereof.

A binding agent may be starch (e.g., cornstarch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, an/or silicone oil.

### Clauses

The disclosure provides the following clauses.

1. A compound of Formula (I):

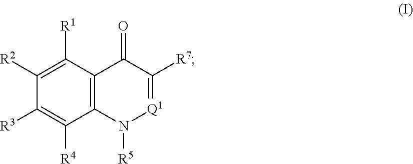

or a pharmaceutically acceptable salt or tautomer thereof, wherein:

Q¹ is CR⁶ or N;

R¹ is selected from the group consisting of H, F, Cl, Br, OR^{1a}, SR^{1a}, N(R^{1a})₂, and C₁-C₆ alkyl; wherein each R^{1a} is independently H, C₁-C₆ alkyl, or C(O)(C₁-C₆ alkyl); wherein the C₁-C₆ alkyl is optionally substituted with one or more halogen or phenyl;

R² is selected from the group consisting of H, F, Cl, Br, and C₁-C₆ alkyl;

R³ is selected from the group consisting of C₁-C₈ alkyl, F, N(R^{3a})(R^{3b}), C₁-C₄ alkyl-N(R^{3a})(R^{3b}), C₃-C₈ cycloalkyl optionally substituted with N(R^{3a})(R^{3b}), CH₂N(R^{3a})CH₂CN, N(R^{3a})CH₂CN, and aryl optionally substituted with R^{3b}; wherein R^{3a} is H or C₁-C₈ alkyl; RR, is H, C₁-C₈ alkyl, C₂-C₈ alkenyl, C₃-C₈ cycloalkyl, C₄-C₈ heterocycloalkyl, C₄-C₈ cycloakenyl, phenyl, indanyl, heteroaryl, or C(O)(C₁-C₆ alkyl); wherein the C₁-C₈ alkyl, C₂-C₈ alkenyl, C₃-C₈ cycloalkyl, C₄-C₈ heterocycloalkyl, C₄-C₈ cycloalkenyl, phenyl, or heteroaryl is optionally substituted with one or more halogen, C₁₋₄ alkyl, OR⁹, NR¹⁰R¹¹, phenyl optionally substituted with C₁-C₄ alkyl, C₃-C₆ cycloalkyl optionally substituted with NR¹⁰R¹¹, or heteroaryl optionally substituted with one or more halogen, C₁-C₄ alkyl, OR⁹, or NR¹⁰R¹¹; wherein when R^{3b} is C₃-C₈ cycloalkyl substituted with at least two C₁-C₄ alkyl substituents, the two C₁-C₄ alkyl substituents together with the carbon atom(s) to which they are attached can form a C₃-C₈ cycloalkyl or C₆-C₁₆ aryl; or wherein R^{3a} and R^{3b} are taken together with the N to which they are attached to form a 3-6 membered heterocycl optionally comprising one or more additional heteroatom selected from N, O and S; that is optionally substituted with one or more OR⁹, NR¹⁰R¹¹, halogen, or C₁-C₄ alkyl;

R⁴ is selected from the group consisting of R^{4a}, F, Cl, Br, OR^{4a}, and N(R^{4a})₂; wherein each R^{4a} is independently H, C₁-C₆ alkyl, C₃-C₆ cycloalkyl, or C(O)(C₁-C₆ alkyl);

R⁵ is selected from the group consisting of H, C₁-C₈ alkyl, C₃-C₈ cycloalkyl, C₄-C₈ cycloalkenyl, C₂-C₈ alkenyl, C₂-C₈ alkynyl, and C(O)(C₁-C₄ alkyl); wherein the C₁-C₈ alkyl, C₃-C₈ cycloalkyl, C₄-C₈ cycloalkenyl, C₂-C₈ alkenyl, or C₂-C₈ alkynyl is optionally substituted with one or more fluoro or phenyl; or wherein R^{4a} and R⁵ are taken together with the atoms to which they are attached to form a 5-8 membered heterocycl optionally substituted with halogen, N(R^{3a})(R^{3b}), OR^{1a}, or optionally substituted C₁-C₃ alkyl; wherein when a carbon atom of the heterocycl has two C₁-C₃ alkyl substituents attached thereto, the two C₁-C₃ alkyl substituents together with the carbon atom to which they are attached can form a 3-8 membered cycloalkyl or heterocycl comprising one or more heteroatoms selected from O, N and S;

R⁶ is selected from the group consisting of H, F, Cl, Br, and C₁-C₆ alkyl;

R⁷ is selected from the group consisting of CN, tetrazolyl, CH₂OR^{7a}, CFHOR^{7a}, CF₂OR^{7a}, CO₂R^{7a}, CON(R^{7b})₂, and C(═NH)—N(R^{7b})₂; wherein R^{7a} is H, C₁-C₆ alkyl or C₃-C₆ cycloalkyl; and each R^{7b} is independently H, OH, C₁-C₆ alkyl, or C₃-C₆ cycloalkyl;

R⁹ is H, C₁-C₆ alkyl, or C(O)(C₁-C6 alkyl); and

R¹⁰ and R¹¹ are each independently selected from H, C₁-C₈ alkyl, C₂-C₈ alkenyl, C₃-C₈ cycloalkyl, C₄-C₈ cycloalkenyl, or C(O)(C₁-C₆ alkyl), wherein the C₁-C₈ alkyl, C₂-C₈ alkenyl, C₃-C₈ cycloalkyl, or C₄-C₈ cycloalkenyl is optionally substituted with one or more F, C₁-C₄ alkyl, optionally substituted phenyl, or optionally substituted heteroaryl.

2. A compound of Formula (II):

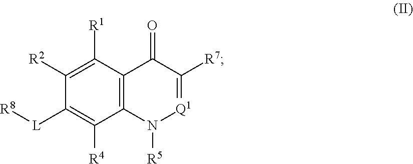

or a pharmaceutically acceptable salt or tautomer thereof, wherein:

Q¹ is CR⁶ or N;

R¹ is selected from the group consisting of H, F, Cl, Br, OR^{1a}, SR^{1a}, N(R^{1a})₂, and C₁-C₆ alkyl; wherein each R^{1a} is independently H, C₁-C₆ alkyl, or C(O)(C₁-C₆ alkyl); wherein the C₁-C₆ alkyl is optionally substituted with one or more halogen or phenyl;

R² is selected from the group consisting of H, F, Cl, Br, and C₁-C₆ alkyl;

R⁴ is selected from the group consisting of R^{4a}, F, Cl, Br, OR^{4a}, and N(R^{4a})₂; wherein each R^{4a} is independently H, C₁-C₆ alkyl, C₃-C₆ cycloalkyl, or C(O)(C₁-C₆ alkyl);

R⁵ is selected from the group consisting of H, C₁-C₈ alkyl, C₃-C₈ cycloalkyl, C₄-C₈ cycloalkenyl, C₂-C₈ alkenyl, C₂-C₈ alkynyl, and C(O)(C₁-C₄ alkyl); wherein the C₁-C₈ alkyl, C₃-C₈ cycloalkyl, C₄-C₈ cycloalkenyl, C₂-C₈ alkenyl, or C₂-C₈ alkynyl is optionally substituted with one or more fluoro or phenyl; or wherein R^{4a} and R⁵ are taken together with the atoms to which they are attached to form a 5-8 membered heterocycl optionally substituted with halogen, N(R^{3a})(R^{3b}), OR^{1a}, or optionally substituted C₁-C₃ alkyl; wherein when a carbon atom of the heterocycl has two C₁-C₃ alkyl substituents attached thereto, the two C₁-C₃ alkyl substituents together with the carbon atom to which they are attached can form a 3-8 membered cycloalkyl or heterocycl comprising one or more heteroatoms selected from O, N and S;

R⁶ is selected from the group consisting of H, F, Cl, Br, and C₁-C₆ alkyl;

R⁷ is selected from the group consisting of CN, tetrazolyl, CH₂OR^{7a}, CFHOR^{7a}, CF₂OR^{7a}, CO₂R^{7a}, CON(R^{7b})₂, and C(═NH)—N(R^{7b})₂; wherein R^{7a} is H, C₁-C₆ alkyl or C₃-C₆ cycloalkyl; and each R^{7b} is independently H, OH, C₁-C₆ alkyl, or C₃-C₆ cycloalkyl;

L is selected from the group consisting of a bond, —(CH₂)₁₋₄—, —C₄-C₈ cycloalkenyl-, —(CH₂)ₙN(R^{La})(CH₂)ₙ—, -cycloalkyl-N(R^{La})—, —(CH₂)ₙO—, -aryl-, -heterocycl-, and -heteroaryl-; wherein L is optionally substituted with one or more halo or C₁-C₄ alkyl; wherein R^{La} is H or C₁-C₈ alkyl; and each n is independently 0 to 4;

R⁸ is selected from the group consisting of C₃-C₈ cycloalkyl-N(R^{8a})(R^{8b}), aryl-C₃-C₈ cycloalkyl-N(R^{8a})(R^{8b}), N(R^{8a})—C₃-C₈ cycloalkyl-aryl, C₄-C₈ cycloalkenyl, OR^{8b}, and N(R^{8a})(R^{8b}); wherein R⁸ is optionally substituted with F or C₁-C₆ alkyl; R^{8a} is H or C₁-C₈ alkyl; and R^{8b} is H or C₁-C₈ alkyl.

3. The compound of clause 1 or clause 2, wherein Q¹ is N.
4. The compound of clause 1 or clause 2, wherein Q¹ is CR⁶.
5. The compound of any one of the preceding clauses, wherein R¹ is selected from the group consisting of H, F, OH, N(R^{1a})₂, and C₁-C₃ alkyl.
6. The compound of any one of the preceding clauses, wherein R¹ is selected from the group consisting of H, NH₂, and CH₃.
7. The compound of any one of the preceding clauses, wherein R¹ is NH₂.
8. The compound of any one of the preceding clauses, wherein R² is F.
9. The compound of any one of the preceding clauses, wherein R³ is selected from the group consisting of N(R^{3a})(R^{3b}), C₁₋₃ alkyl-N(R^{3a})(R^{3b}), CH₂N(R^{3a})CH₂CN, N(R^{3a})CH₂CN, aryl optionally substituted with R^{3b}, and C₃-C₈ cycloalkyl optionally substituted with NR¹⁰R¹¹.
10. The compound of any one of the preceding clauses, wherein R³ is N(R^{3a})(R^{3b}).
11. The compound of any one of the preceding clauses, wherein R³ is NH(R^{3b}).
12. The compound of any one of the preceding clauses, wherein R³ is NH(R^{3b}) and R^{3b} is selected from the group consisting of C₃-C₈ cycloalkyl, C₄-C₈ heterocycloalkyl, C₃-C₈ cycloalkyl substituted with NR¹⁰R¹¹, C₃-C₈ cycloalkyl substituted with heteroaryl, C₁-C₈ alkyl substituted with phenyl, C₁-C₈ alkyl substituted with NR¹⁰R¹¹, indanyl, and phenyl optionally substituted with C₃-C₆ cycloalkyl optionally substituted with NR¹⁰R¹¹.
13. The compound of any one of the preceding clauses, wherein R³ is NH(R^{3b}) and R^{3b} is selected from the group consisting of C₃-C₈ cycloalkyl substituted with NHR¹¹, C₁-C₈ alkyl substituted with NHR¹¹, and phenyl optionally substituted with C₃-C₆ cycloalkyl optionally substituted with NHR¹¹, wherein R¹¹ is selected from the group consisting of H and C₃-C₈ cycloalkyl substituted with an optionally substituted phenyl.
14. The compound of any one of the preceding clauses, wherein R³ is NH(R^{3b}) and R^{3b} is C₃-C₈ cycloalkyl or C₄-C₈ heterocycloalkyl.
15. The compound of any one of the preceding clauses, wherein R³ is selected from the group consisting of:

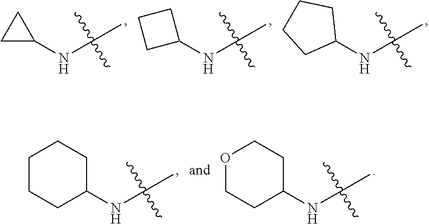 [SMILES: *NC1CC1 ; *NC1CCC1 ; *NC1CCCC1 ; *NC1CCCCC1 ; *NC1CCOCC1]

16. The compound of any one of the preceding clauses, wherein R³ is N(R^{3a})(R^{3b}) and R^{3b} is C₃-C₈ cycloalkyl optionally substituted with an optionally substituted phenyl.
17. The compound of any one of the preceding clauses, wherein R³ is NH(R^{3b}) and R^{3b} is cyclopropyl substituted with phenyl or cyclobutyl substituted with phenyl.
18. The compound of any one of the preceding clauses, wherein R³ is selected from the group consisting of:

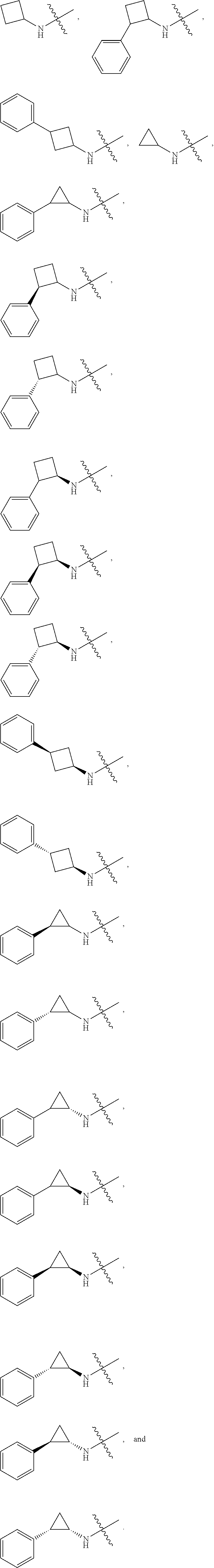

19. The compound of any one of the preceding clauses, wherein R³ is NH(R^{3a})(R^{3b}) and R^{3b} is C₃-C₈ cycloalkyl optionally substituted with one or more C₁-C₄ alkyl and phenyl optionally substituted with C₁-C₄ alkyl.
20. The compound of any one of the preceding clauses, wherein R³ is NH(R^{3b}) and R^{3b} is cyclopropyl substituted with one or more methyl and one or more phenyl.
21. The compound of any one of the preceding clauses, wherein R³ is NH(R^{3b}) and R^{3b} is C₃-C₈ cycloalkyl substituted with NR¹⁰R¹¹.
22. The compound of any one of the preceding clauses, wherein R³ is NH(R^{3b}) and R^{3b} is C₃-C₈ cycloalkyl substituted with NHR¹¹ and R¹¹ is C₃-C₈ cycloalkyl substituted with an optionally substituted phenyl.
23. The compound of any one of the preceding clauses, wherein R³ is selected from the group consisting of:

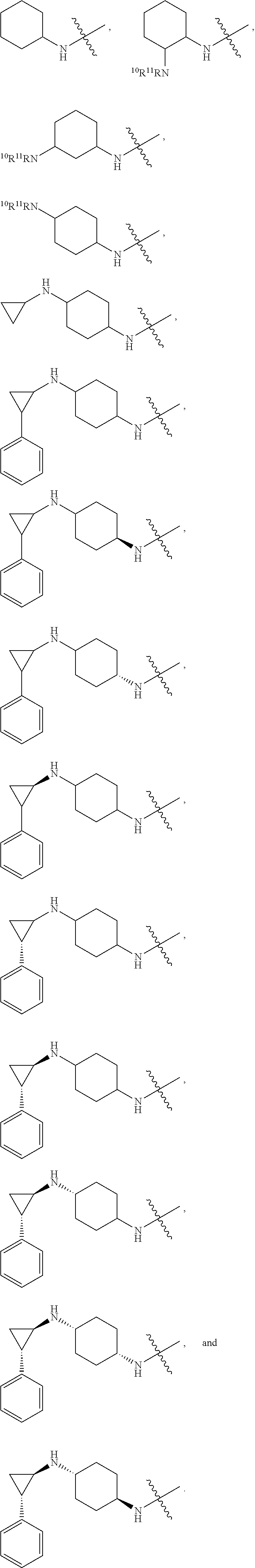

24. The compound of any one of the preceding clauses, wherein R³ is NH(R^{3b}) and R^{3b} is C₃-C₈ cycloalkyl substituted with heteroaryl.
25. The compound of any one of the preceding clauses wherein R³ is selected from the group consisting of:

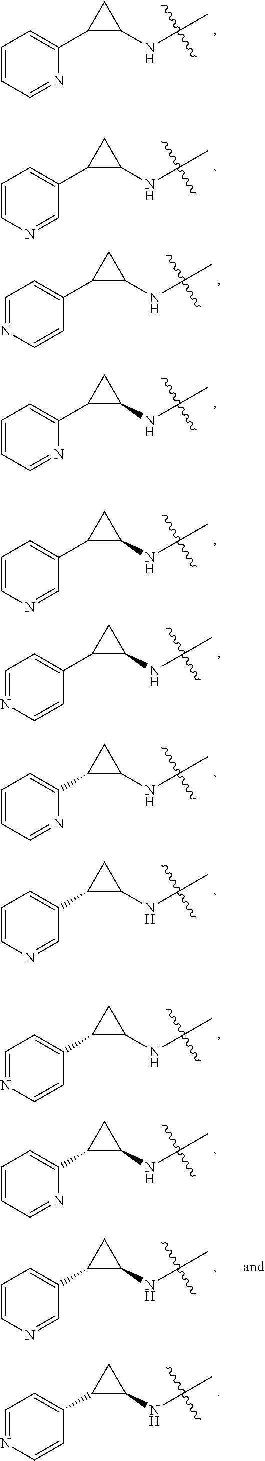

26. The compound of any one of the preceding clauses, wherein R³ is NH(R^{3b}) and R^{3b} is C₁-C₈ alkyl substituted with phenyl.
27. The compound of any one of the preceding clauses, wherein R³ is selected from the group consisting of:

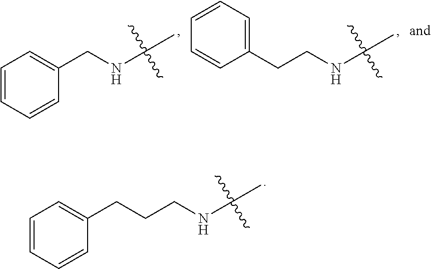

28. The compound of any one of the preceding clauses, wherein R³ is NH(R^{3b}) and R^{3b} is C₁-C₈ alkyl substituted with NR¹⁰R¹¹.
29. The compound of any one of the preceding clauses, wherein R³ is NH(R^{3b}) and R^{3b} is C₁-C₈ alkyl substituted with NHR¹¹ and R¹¹ is C₃-C₈ cycloalkyl substituted with an optionally substituted phenyl.
30. The compound of any one of the preceding clauses, wherein R³ is selected from the group consisting of:

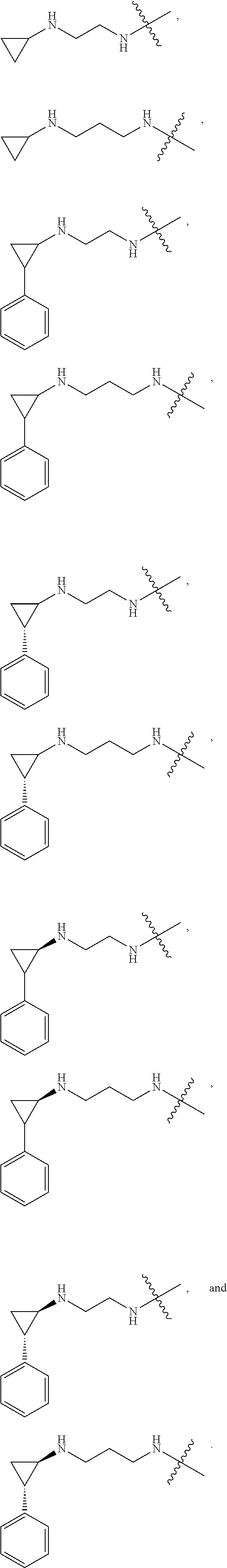

31. The compound of any one of the preceding clauses, wherein R³ is NH(R^{3b}) and R^{3b} is indanyl or phenyl, wherein the phenyl is optionally substituted with C₃-C₆ cycloalkyl optionally substituted with NR¹⁰R¹¹.
32. The compound of any one of the preceding clauses, wherein R³ is selected from the group consisting of:

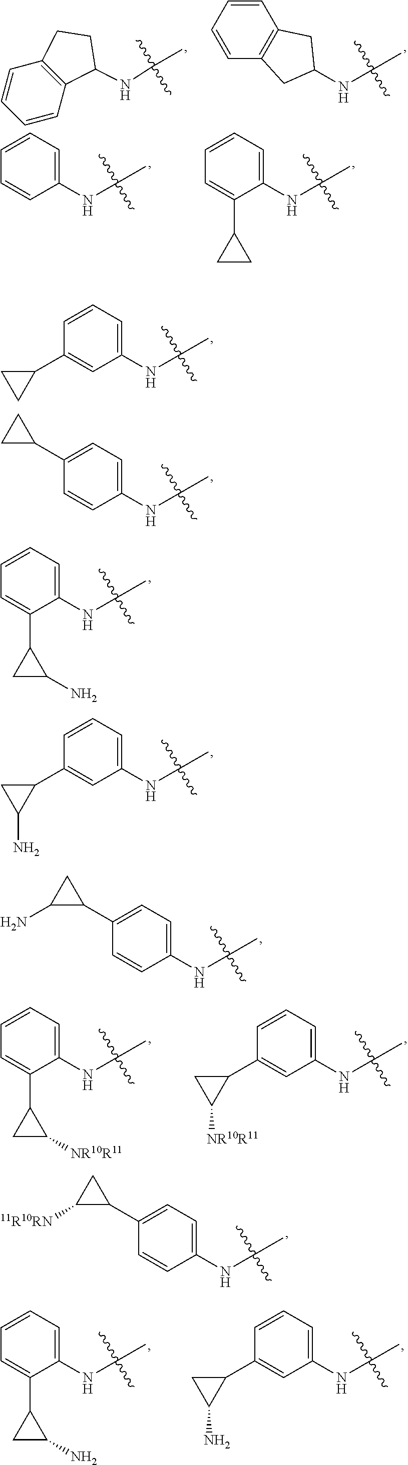

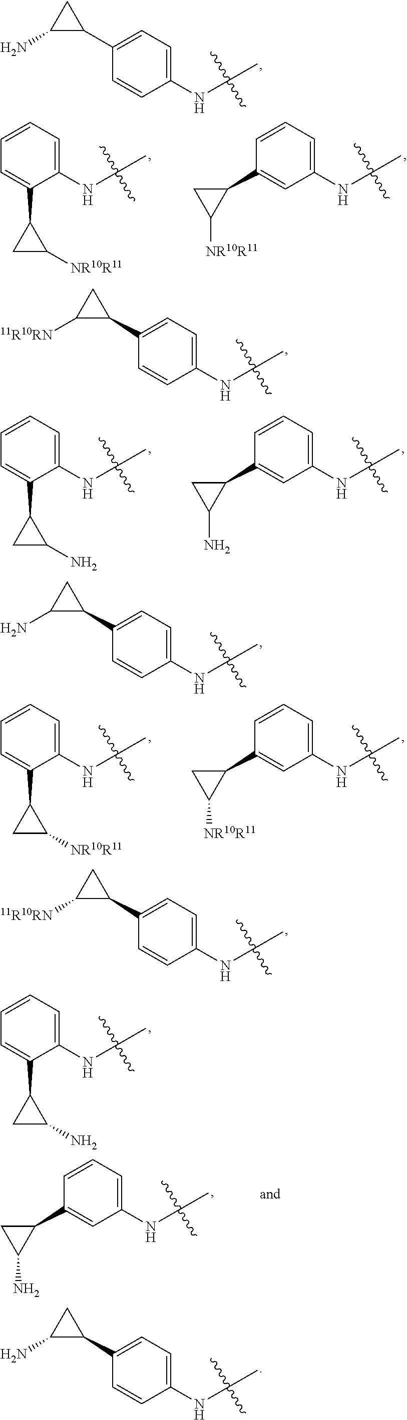

33. The compound of any one of the preceding clauses, wherein R³ is N(R^{3a})(R^{3b}) wherein R^{3a} and R^{3b} are taken together with the N to which they are attached to form a 3-6 membered heterocycl substituted with one or more NR¹⁰R¹¹.
34. The compound of any one of the preceding clauses, wherein R³ is N(R^{3a})(R^{3b}) wherein R^{3a} and R^{3b} are taken together with the N to which they are attached to form a 6 membered heterocycl substituted with NR¹⁰R¹¹ wherein R¹⁰ is H and R¹¹ is C₃-C₈ cycloalkyl substituted with an optionally substituted phenyl.
35. The compound of any one of the preceding clauses, wherein R³ is selected from the group consisting of:

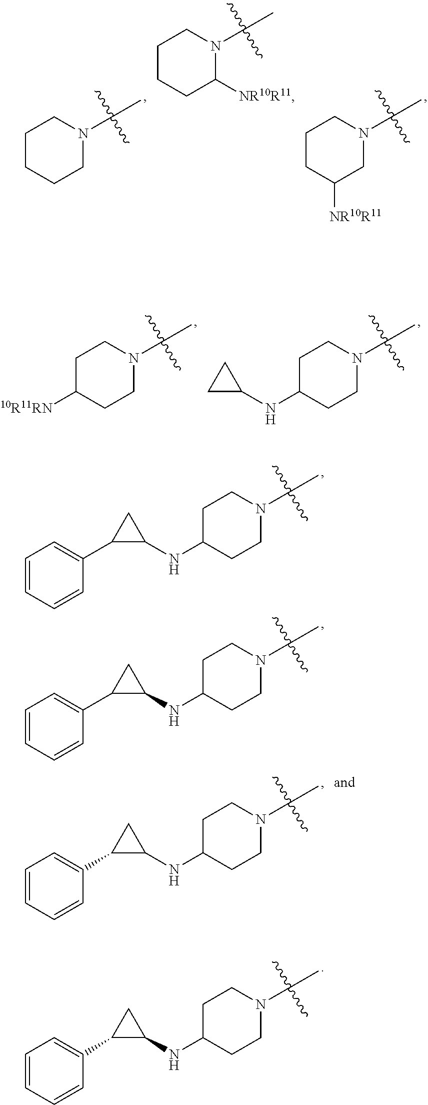

36. The compound of any one of the preceding clauses, wherein R³ is NH(R^{3b}) and R^{3b} is C₃-C₈ cycloalkyl substituted with two C₁-C₄ alkyl substitutents and optionally further substituted with phenyl; and wherein the two C₁-C₄ alkyl substituents together with the carbon atom(s) to which they are attached form a C₃-C₈ cycloalkyl or C₆-C₁₆ aryl.
37. The compound of any one of the preceding clauses, wherein R³ is elected from the group consisting of:

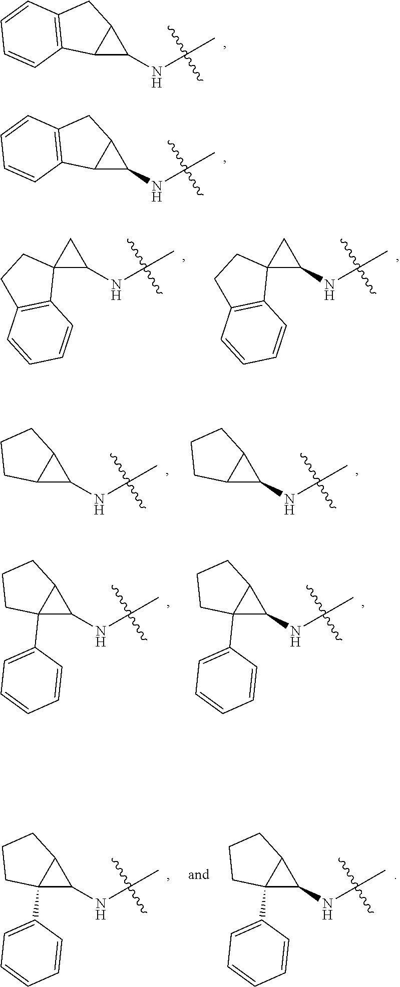

38. The compound of any one of the preceding clauses, wherein R³ is C₁-C₈ alkyl-N(R^{3a})(R^{3b}).
39. The compound of any one of the preceding clauses, wherein R³ is C₁-C₄ alkyl-NH(R^{3b}) and R^{3b} is C₃-C₈ cycloalkyl substituted with phenyl.
40. The compound of any one of the preceding clauses, wherein R³ is selected from the group consisting of:

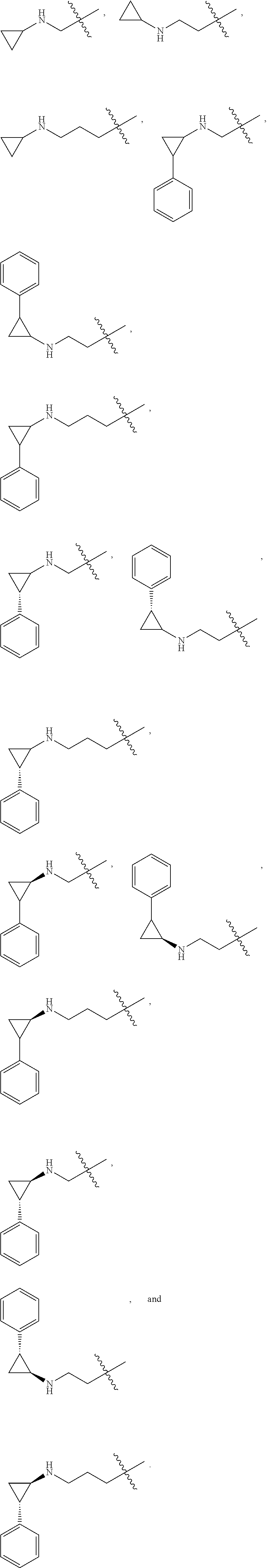

41. The compound of any one of the preceding clauses, wherein R³ is selected from the group consisting of CH₂N(R^{3a})CH₂CN and N(R^{3a})CH₂CN.
42. The compound of any one of the preceding clauses, wherein R³ is selected from the group consisting of CH₂NHCH₂CN, CH₂N(CH₃)CH₂CN, NHCH₂CN, and N(CH₃)CH₂CN.
43. The compound of any one of the preceding clauses, wherein R³ is aryl optionally substituted with R^{3b}.
44. The compound of any one of the preceding clauses, wherein R³ is aryl optionally substituted with R^{3b} and R^{3b} is C₃-C₆ cycloalkyl optionally substituted with NR¹⁰R¹¹.
45. The compound of any one of the preceding clauses, wherein R³ is selected from the group consisting of:

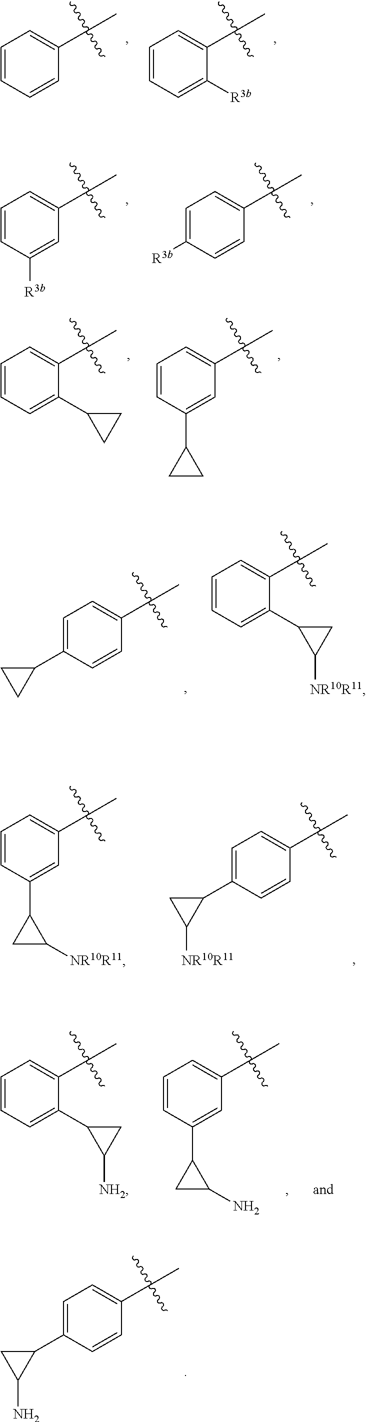

46. The compound of any one of the preceding clauses, wherein R³ is C₃-C₈ cycloalkyl optionally substituted with NR¹⁰R¹¹.
47. The compound of any one of the preceding clauses, wherein R³ is selected from the group consisting of:

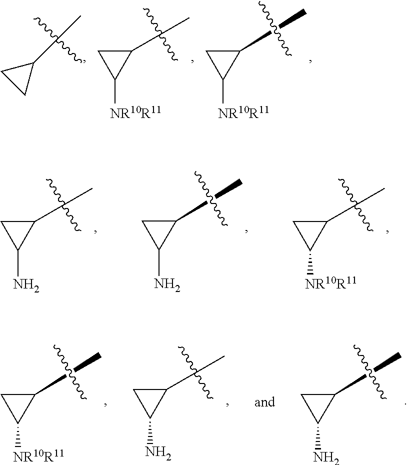

48. The compound of any one of the preceding clauses, wherein R⁴ is selected from the group consisting of H and F.
49. The compound of any one of the preceding clauses, wherein R⁴ is H.
50. The compound of any one of the preceding clauses, wherein R⁵ is selected from the group consisting of C₁-C₆ alkyl and C₃-C₆ cycloalkyl.
51. The compound of any one of the preceding clauses, wherein R⁵ is selected from the group consisting of CH₃, CH₂CH₃, CH₂CH₂CH₃, CH(CH₃)₂, CH(CH₂CH₃)₂, and cyclopropyl.
52. The compound of any one of the preceding clauses, wherein R⁵ is CH₂CH₃.
53. The compound of any one of the preceding clauses, wherein R⁶ is H.
54. The compound of any one of the preceding clauses, wherein R⁷ is selected from the group consisting of CN, tetrazolyl, CH₂OR^{7a}, CO₂R^{7a}, CON(R^{7b})₂, and C(═NH)—N(R^{7b})₂.
55. The compound of any one of the preceding clauses, wherein R⁷ is selected from the group consisting of CN, CH₂OH, CO₂H, CO₂CH₃, CO₂CH₂CH₃, CONH₂, CONHOH, CONHCH₃, CON(CH₃)₂, and C(═NH)—NHOH.
56. The compound of any one of the preceding clauses, wherein R⁷ is selected from the group consisting of CN and CO₂H.
57. The compound of any one of the preceding clauses, wherein L is selected from the group consisting of a bond, —(CH₂)₁₋₄—, —(CH₂)ₙN(R^{La})(CH₂)ₙ—, and -cycloalkyl-N(R^{La})—.
58. The compound of any one of the preceding clauses, wherein L is selected from the group consisting of a bond, —CH₂—, —N(R^{La})CH₂—, —CH₂N(R^{La})—, -cyclopropyl-N(R^{La})—, and -cyclobutyl-N(R^{La})—.
59. The compound of any one of the preceding clauses, wherein R⁸ is selected from the group consisting of C₃-C₈ cycloalkyl-N(R^{8a})(R^{8b}), N(R^{8a})—C₃-C₈ cycloalkyl-aryl, and aryl-C₃-C₈ cycloalkyl-N(R^{8a})(R^{8a}).
60. The compound of any one of the preceding clauses, wherein R⁸ is selected from the group consisting of cyclopropyl-NH₂, cyclobutyl-NH₂, phenyl-cyclopropyl-NH₂, phenyl-cyclobutyl-NH₂, NH-cyclopropyl-phenyl, and NH-cyclobutyl-phenyl.
61. The compound of any one of the preceding clauses, wherein the compound is of any one of Formulae (Ia), (Ib), (Ic), (Id), (Ie), (If), (Ig), (Ih), (Ii), (Ij), or (Ik):

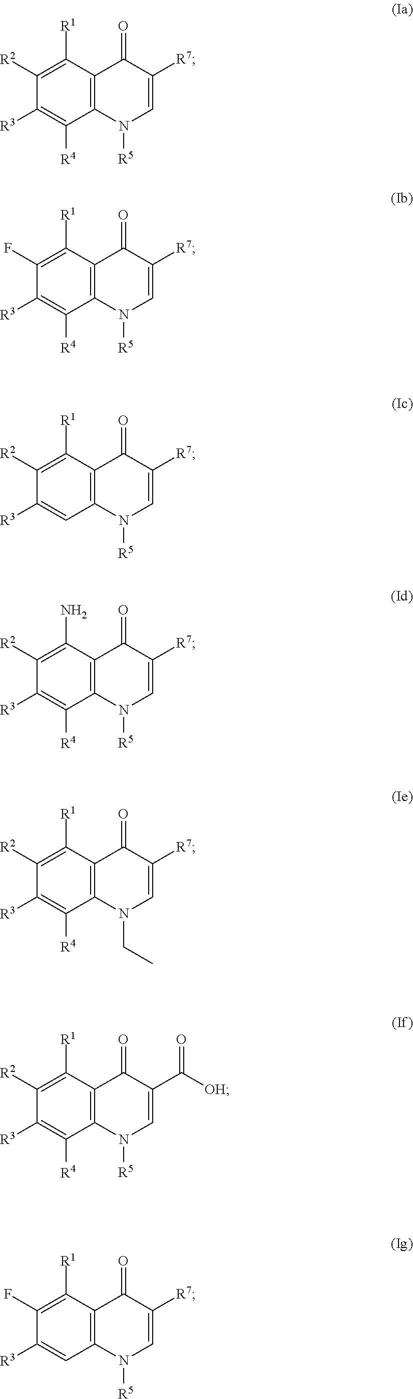

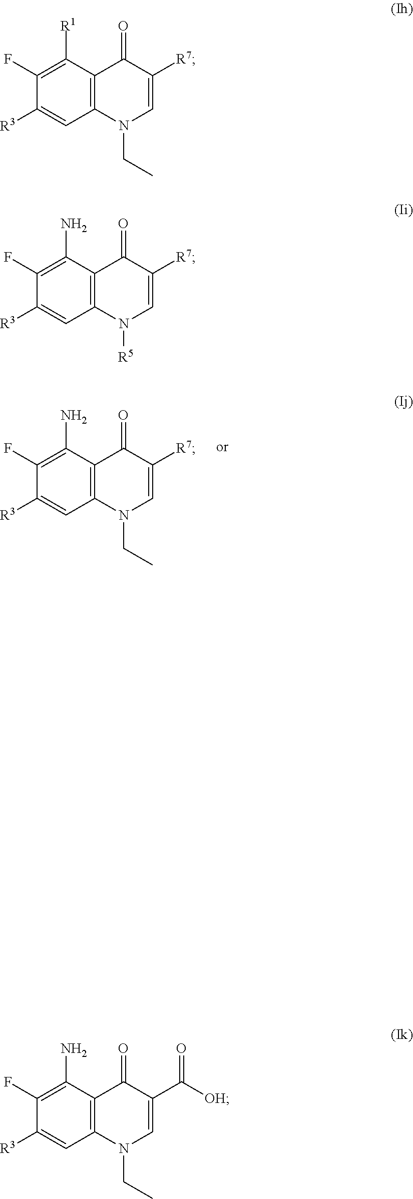

or a pharmaceutically acceptable salt or tautomer thereof, wherein R¹, R², R³, R⁴, R⁵, and R⁷ are as described herein.
62. The compound of any one of the preceding clauses, wherein the compound is any one of Formulae (IIIa), (IIIb), (IIIc), or (IIId):

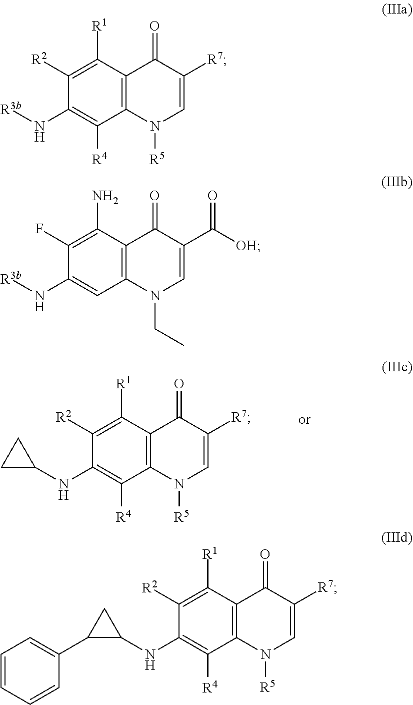

or a pharmaceutically acceptable salt or tautomer thereof, wherein R¹, R², R^{3b}, R⁴, R⁵ and R⁷ are as described herein.
63. The compound of any one of the preceding clauses, wherein the compound is any one of Formulae (IVa), (IVb), (IVc), or (IVd):

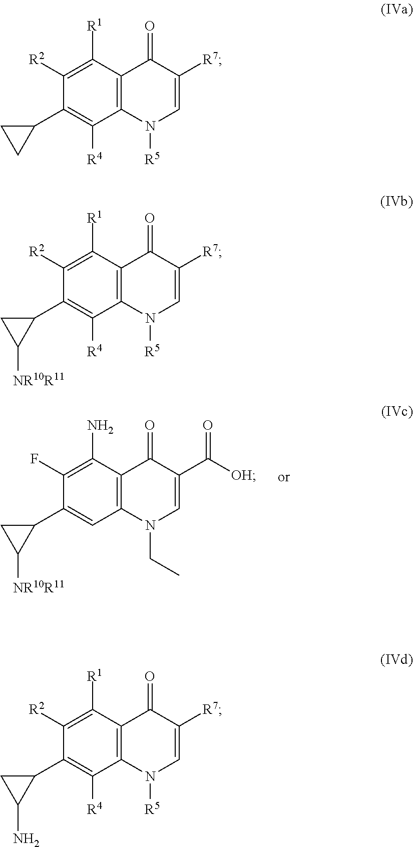

or a pharmaceutically acceptable salt or tautomer thereof, wherein R¹, R², R⁴, R⁵, R⁷, R¹⁰ and R¹¹ are as described herein.
64. The compound of any one of the preceding clauses, wherein the compound is any one of Formulae (Va), (Vb), (Vc), or (Vd):

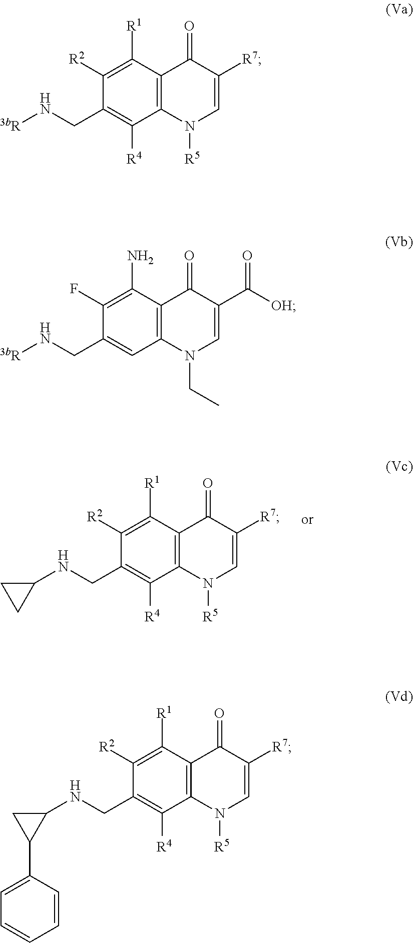

or a pharmaceutically acceptable salt or tautomer thereof, wherein R¹, R², R^{3b}, R⁴, R⁵ and R⁷ are as described herein.
65. The compound of any one of the preceding clauses, wherein the compound is any one of Formulae (VIa), (VIb), (VIc), (VId), (VIe), (VII), (VIg), (VIh), (VIi), (VIj), or (VIk):

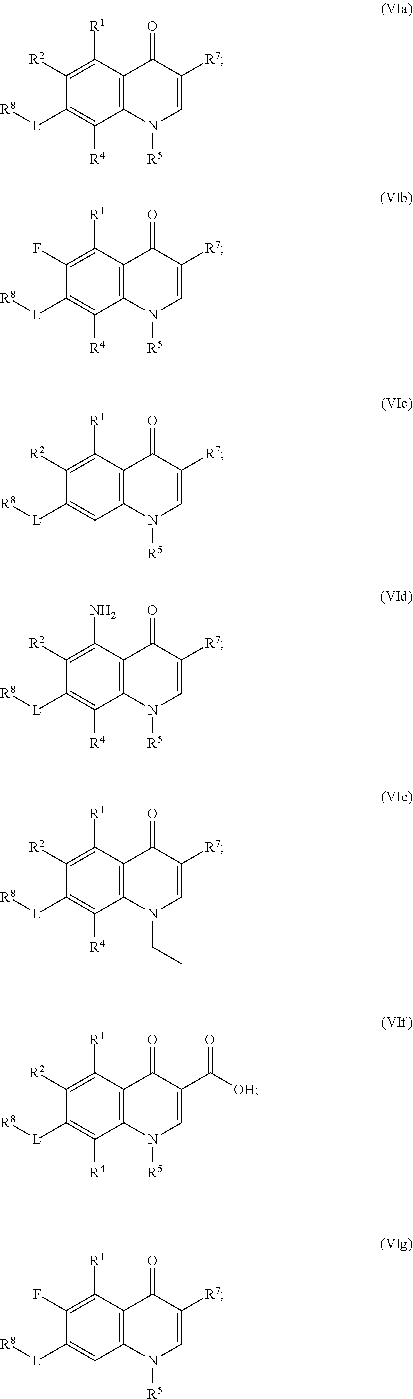

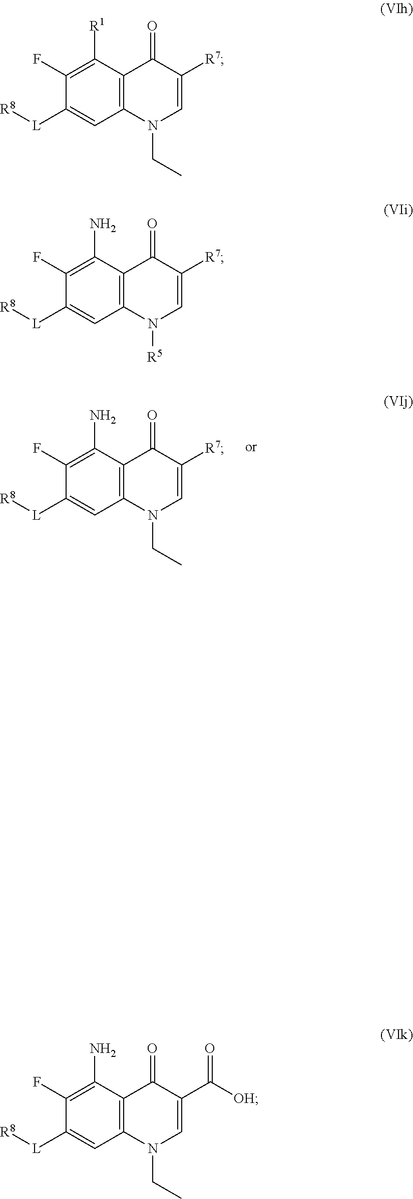

or a pharmaceutically acceptable salt or tautomer thereof, wherein R¹, R², R⁴, R⁵, R⁷, L and R⁸ are as described herein.
66. The compound of any one of the preceding clauses, wherein the compound is any one of Formulae (VIIa) or (VIIb):

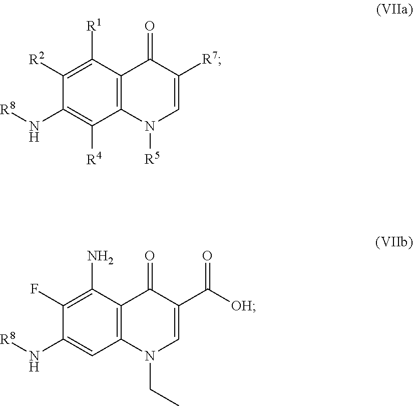

or a pharmaceutically acceptable salt or tautomer thereof, wherein R¹, R², R⁴, R⁵, R⁷ and R⁸ are as described herein.
67. The compound of any one of the preceding clauses, wherein the compound is any one of Formulae (VIIIa) or (VIIIb):

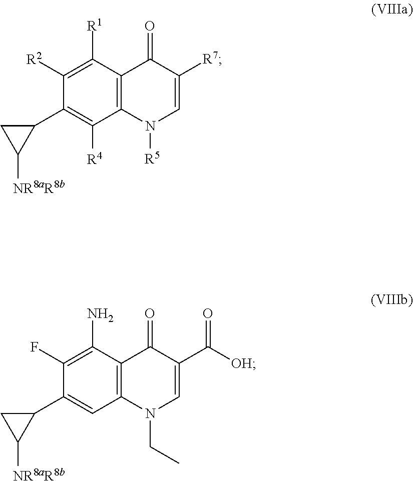

or a pharmaceutically acceptable salt or tautomer thereof, wherein R¹, R², R⁴, R⁵, R⁷, R^{8a} and R^{8b} are as described herein.
68. The compound of any one of the preceding clauses, wherein the compound is any one of Formulae (IXa) or (IXb):

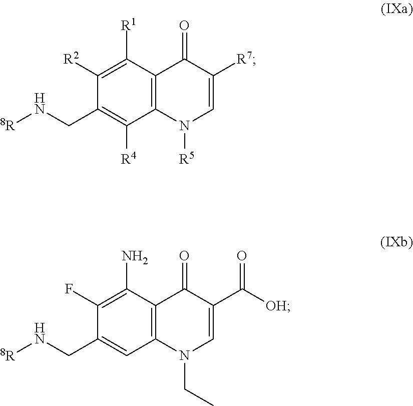

or a pharmaceutically acceptable salt or tautomer thereof, wherein R¹, R², R⁴, R⁵, R⁷ and R⁸ are as described herein.
69. A compound selected from the group consisting of compound I-1 to compound I-49 from Table A.
70. A pharmaceutical composition comprising a compound of any one of the preceding clauses, or a pharmaceutically acceptable salt or tautomer thereof, and a pharmaceutically acceptable carrier.
71. The pharmaceutical composition of clause 70, further comprising an additional pharmaceutically active agent.
72. A method of expanding a population of cochlear cells in a cochlear tissue comprising a parent population, the method comprising contacting the cochlear tissue with a compound of any one of the preceding clauses, or a pharmaceutically acceptable salt or tautomer thereof or a pharmaceutical composition of any one of the preceding clauses.
73. The method of clause 72, wherein the cochlear tissue is in a subject.
74. The method of any one of the preceding clauses, wherein the contacting the cochlear tissue with the composition is achieved by administering the composition trans-tympanically to the subject.
75. The method of any one of the preceding clauses, wherein contacting the cochlear tissue with the composition results in improved auditory functioning of the subject.
76. A method of facilitating the generation of tissue cells, the method comprising administering or causing to be administered to a stem cell population a compound of any one of the preceding clauses, or a pharmaceutically acceptable salt or tautomer thereof or a pharmaceutical composition of any one of the preceding clauses.
77. The method of clause 76, wherein the tissue cells are cochlear cells.
78. The method of clause any one of the preceding clauses, wherein the tissue cells are inner ear hair cells.
79. A method of treating a subject who has, or is at risk of developing, a disease associated with absence or lack of certain tissue cells, comprising administering or causing to be administered to a stem cell population a compound of any one of the preceding clauses, or a pharmaceutically acceptable salt or tautomer thereof or a pharmaceutical composition of any one of the preceding clauses.
80. The method of clause 79, wherein the tissue cells are cochlear cells.
81. The method of any one of the preceding clauses, wherein the tissue cells are inner ear hair cells.
82. A method of treating a subject who has, or is at risk of developing, hearing loss, the method comprising administering a compound of any one of the preceding clauses, or a pharmaceutically acceptable salt or tautomer thereof or a pharmaceutical composition of any one of the preceding clauses.
83. The method of clause 82, wherein the compound is administered trans-tympanically to a cochlear tissue of the subject.
84. A method of facilitating the generation of inner ear hair cells, the method comprising: administering a compound of any one of the preceding clauses or a pharmaceutically acceptable salt thereof, alone or in combination with an additional pharmaceutically active agent, to expand the stem cell population of cochlear tissue.
85. A method of regenerating or improving hearing in a mammal, the method comprising administering a compound of any one of the preceding clauses, or a pharmaceutically acceptable salt or tautomer thereof, alone or in combination with an additional pharmaceutically active agent.
86. The method of clauses 84 or 85, wherein the administration is to a stem cell population is of an in vivo subject.
87. A method of generating inner ear hair cells, the method comprising administering a compound of any one of the preceding clauses, or a pharmaceutically acceptable salt or tautomer thereof, alone or in combination with an additional pharmaceutically active agent, wherein the method proliferates Lgr5⁺ cells in an initial population in vivo, resulting in an expanded population of Lgr5⁺ cells, resulting in generation of inner ear hair cells.
88. A method of facilitating generation of intestinal cells, the method comprising administering a compound of any one of the preceding clauses or a pharmaceutically acceptable salt thereof, alone or in combination with an additional pharmaceutically active agent, to expand the stem cell population of intestinal epithelia.
89. The method of clause 88, wherein the intestinal epithelia is regenerated.
90. The method of any one of the preceding clauses, wherein the method is a treatment for promoting repair of damaged mucosa related to chemotherapy-induced gastrointestinal mucositis, Graph Versus Host Disease, gastric ulcer, Crohns, or ulcerative colitis.
91. A method of expanding Lgr5⁺ cell population of intestinal epithelia, the method comprising: administering a compound of any one of the preceding clauses or a pharmaceutically acceptable salt thereof, alone or in combination with an additional pharmaceutical agent.
92. A method of use of a compound of any one of the preceding clauses, or a pharmaceutically acceptable salt or tautomer thereof, alone or in combination with an additional pharmaceutically active agent to regenerate Lgr5⁺ cell population intestinal cells in a mammal.
93. The method of clause 92, wherein the method is a treatment for promoting the repair of damaged mucosa related to chemotherapy-induced gastrointestinal mucositis, Graph Versus Host Disease, gastric ulcer, Crohns, or ulcerative colitis.
94. A method of proliferating Lgr5⁺ epithelial cells in in vivo, the method comprising: administering a compound of any one of the preceding clauses or a pharmaceutically acceptable salt thereof.
95. A method for expanding a population of vestibular cells in a vestibular tissue comprising contacting the vestibular tissue with (i) a compound of any one of the preceding clauses or a pharmaceutically acceptable salt thereof, and (ii) an additional pharmaceutically active agent to form an expanded population of cells in the vestibular tissue.
96. A method of treating a subject who has, or is at risk of developing, vestibular diseases, alopecia, oncology, acute myeloid leukemia, inflammation, Alzheimer's disease, Huntington's disease, Friedreick's ataxia, depression, anxiety, manic episodes of bipolar/mood disorders, Parkinson's, diabetes, bacterial infection, Anti-Trypanosoma brucei, ischemia, heart disease, vascular degeneration, and/or platelet aggregation, the method comprising administering a compound of any one of the preceding clauses or a pharmaceutically acceptable salt thereof, alone or in combination with an additional pharmaceutically active agent.
97. The method of any one of the preceding clauses, wherein the compound of any one of the preceding clauses functions as an inhibitor of at least one selected from the group consisting of FOXO-1, GSK3α/β and LSD-1.
98. A method of inhibiting LSD, GSK3, and/or FOXO in a cell, the method comprising contacting the cell with a compound of any one of the preceding clauses or a pharmaceutically acceptable salt thereof.
99. The method of any one of the preceding clauses, wherein the compound of any one of the preceding clauses functions as a cell cycle progression pathway agonist.
100. The method of any one of the preceding clauses, wherein the additional pharmaceutical agent is an HDAC inhibitor and/or a poloxamer.
101. A system for treating a subject who has, or is at risk of developing, a disease associated with absence or lack of certain tissue cells, comprising administering:

a compound of any one of the preceding clauses, or a pharmaceutically acceptable salt or tautomer thereof; and a trans-tympanic administrative device.

102. A compound of any one of the preceding clauses, or a pharmaceutically acceptable salt or tautomer thereof, for use in treating a subject who has, or is at risk of developing, a disease associated with absence or lack of certain tissue cells.
103. A compound of any one of the preceding clauses, or a pharmaceutically acceptable salt or tautomer thereof, for use in treating a subject who has, or is at risk of developing, hearing loss.
104. Use of a compound of any one of the preceding clauses, or a pharmaceutically acceptable salt or tautomer thereof, in the manufacture of a medicament for treating a subject who has, or is at risk of developing, a disease associated with absence or lack of certain tissue cells.
105. Use of a compound of any one of the preceding clauses, or a pharmaceutically acceptable salt or tautomer thereof, in the manufacture of a medicament for treating a subject who has, or is at risk of developing, hearing loss.
106. Use of a compound of any one of the preceding clauses, or a pharmaceutically acceptable salt or tautomer thereof, in the manufacture of a medicament for treating a subject who has, or is at risk of developing a disease responding to LSD inhibition, GSK3 inhibition, and/or FOXO inhibition.
107. Use of a compound of any one of the preceding clauses, or a pharmaceutically acceptable salt or tautomer thereof, in the manufacture of a medicament for treating a subject who has, or is at risk of developing vestibular diseases, alopecia, oncology, acute myeloid leukemia, inflammation, Alzheimer's disease, Huntington's disease, Friedreick's ataxia, depression, anxiety, manic episodes of bipolar/mood disorders, Parkinson's, diabetes, bacterial infection, Anti-Trypanosoma brucei, ischemia, heart disease, vascular degeneration, and/or platelet aggregation.

### EMBODIMENTS

In further embodiments, enumerated as embodiments 1-128 below, the present disclosure includes:

Embodiment 1. A Compound of Formula (I):

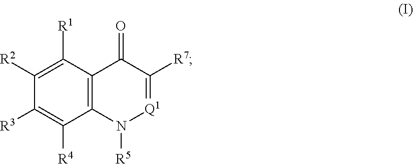

or a pharmaceutically acceptable salt or tautomer thereof, wherein:

Q¹ is CR⁶ or N;

R¹ is selected from the group consisting of H, F, Cl, Br, NO₂, OR^{1a}, SR^{1a}, N(R^{1a})₂, and C₁-C₆ alkyl; wherein each R^{1a} is independently H, C₁-C₆ alkyl, or —C(═O)—(C₁-C₆ alkyl); wherein the C₁-C₆ alkyl is optionally substituted with one or more halogen or phenyl;

R² is selected from the group consisting of H, F, Cl, Br, C₁-C₆ alkyl, and NR¹⁰R¹¹;

R³ is selected from the group consisting of -L-R⁸, C₁-C₈ alkyl, F, N(R^{3a})(R^{3b}), C₁-C₄ alkyl-N(R^{3a})(R^{3b}), OR^{3b}, C₃-C₈ cycloalkyl optionally substituted with N(R^{3a})(R^{3b}), CH₂N(R^{3a})CH₂CN, N(R^{3a})CH₂CN, and aryl optionally substituted with R^{3b}; wherein R^{3a} is H or C₁-C₈ alkyl; R^{3b} is H, C₁-C₈ alkyl, C₂-C₈ alkenyl, C₃-C₈ cycloalkyl, C₄-C₈ heterocycloalkyl, C₄-C₈ cycloalkenyl, phenyl, indanyl, heteroaryl, or —C(═O)—(C₁-C₆ alkyl); wherein the C₁-C₈ alkyl, C₂-C₈ alkenyl, C₃-C₈ cycloalkyl, C₄-C₈ heterocycloalkyl, C₄-C₈ cycloalkenyl, phenyl, heteroaryl, or —C(═O)—(C₁-C₆ alkyl) is optionally substituted with one or more halogen, C₁₋₄ alkyl, OR⁹, NR¹⁰R¹¹, phenyl optionally substituted with C₁-C₄ alkyl, C₃-C₆ cycloalkyl optionally substituted with NR¹⁰R¹¹, or heteroaryl optionally substituted with one or more halogen, C₁-C₄ alkyl, OR⁹, or NR¹⁰R¹¹; wherein when R^{3b} is C₃-C₈ cycloalkyl substituted with at least two C₁-C₄ alkyl substituents, the two C₁-C₄ alkyl substituents together with the carbon atom(s) to which they are attached can form a C₃-C₈ cycloalkyl or C₆-C₁₆ aryl; or wherein R^{3a} and R^{3b} are taken together with the N to which they are attached to form a 3-6 membered heterocycl optionally comprising one or more additional heteroatom selected from N, O and S; that is optionally substituted with one or more OR⁹, NR¹⁰R¹¹, halogen, or C₁-C₄ alkyl;

R⁴ is selected from the group consisting of R^{4a}, F, Cl, Br, OR^{4a}, and N(R^{4a})₂; wherein each R^{4a} is independently H, C₁-C₆ alkyl, C₃-C₆ cycloalkyl, or —C(═O)—(C₁-C₆ alkyl);

R⁵ is selected from the group consisting of H, C₁-C₈ alkyl, C₃-C₈ cycloalkyl, C₄-C₈ cycloalkenyl, C₂-C₈ alkenyl, C₂-C₈ alkynyl, and —C(═O)—(C₁-C₄ alkyl); wherein the C₁-C₈ alkyl, C₃-C₈ cycloalkyl, C₄-C₈ cycloalkenyl, C₂-C₈ alkenyl, or C₂-C₈ alkynyl is optionally substituted with one or more fluoro, phenyl, or OR^{1a}; or wherein R^{4a} and R⁵ are taken together with the atoms to which they are attached to form a 5-8 membered heterocycl optionally substituted with halogen, N(R^{3a})(R^{3b}), OR^{1a}, or optionally substituted C₁-C₃ alkyl; wherein when a carbon atom of the heterocycl has two C₁-C₃ alkyl substituents attached thereto, the two C₁-C₃ alkyl substituents together with the carbon atom to which they are attached can form a 3-8 membered cycloalkyl or heterocycl comprising one or more heteroatoms selected from O, N and S;

R⁶ is selected from the group consisting of H, F, Cl, Br, and C₁-C₆ alkyl;

R⁷ is selected from the group consisting of CN, tetrazolyl, CH₂OR^{7a}, CFHOR^{7a}, CF₂OR^{7a}, CO₂R^{7a}, CON(R^{7b})₂, and C(═NH)—N(R^{7b})₂; wherein R^{7a} is H, C₁-C₆ alkyl or C₃-C₆ cycloalkyl; and each R^{7b} is independently H, OH, C₁-C₆ alkyl, or C₃-C₆ cycloalkyl;

L is selected from the group consisting of a bond, —(CH₂)₁₋₄—, —C₄-C₈ cycloalkenyl-, —(CH₂)ₙN(R^{La})(CH₂)ₙ—, -cycloalkyl-N(R^{La})—, —(CH₂)ₙO—, -aryl-, -heterocycl-, and -heteroaryl-; wherein L is optionally substituted with one or more halo or C₁-C₄ alkyl; wherein R^{La} is H or C₁-C₈ alkyl; and each n is independently 0 to 4;

R⁸ is selected from the group consisting of C₃-C₈ cycloalkyl-N(R^{8a})(R^{8b}), aryl-C₃-C₈ cycloalkyl-N(R^{8a})(R^{8b}), N(R^{8a})—C₃-C₈ cycloalkyl-aryl, C₄-C₈ cycloalkenyl, OR^{8b}, and N(R^{8a})(R^{8b}); wherein R⁸ is optionally substituted with F or C₁-C₆ alkyl; R^{8a} is H or C₁-C₈ alkyl; and R^{8b} is H or C₁-C₈ alkyl;

R⁹ is H, C₁-C₆ alkyl, or —C(═O)—(C₁-C₆ alkyl); and

R¹⁰ and R¹¹ are each independently selected from H, C₁-C₈ alkyl, C₂-C₈ alkenyl, C₃-C₈ cycloalkyl, C₄-C₈ cycloalkenyl, or —C(═O)—(C₁-C₆ alkyl), wherein the C₁-C₈ alkyl, C₂-C₈ alkenyl, C₃-C₈ cycloalkyl, or C₄-C₈ cycloalkenyl is optionally substituted with one or more F, C₁-C₄ alkyl, optionally substituted phenyl, or optionally substituted heteroaryl, or indanyl.

Embodiment 2. A compound of Formula (II):

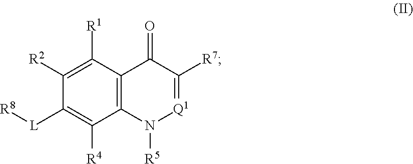

or a pharmaceutically acceptable salt or tautomer thereof, wherein:

Q¹ is CR⁶ or N;

R¹ is selected from the group consisting of H, F, Cl, Br, NO₂, OR^{1a}, SR^{1a}, N(R^{1a})₂, and C₁-C₆ alkyl; wherein each R^{1a} is independently H, C₁-C₆ alkyl, or —C(═O)—(C₁-C₆ alkyl); wherein the C₁-C₆ alkyl is optionally substituted with one or more halogen or phenyl;

R² is selected from the group consisting of H, F, Cl, Br, C₁-C₆ alkyl, and NR¹⁰R¹¹;

R⁴ is selected from the group consisting of R^{4a}, F, Cl, Br, OR^{4a}, and N(R^{4a})₂; wherein each R^{4a} is independently H, C₁-C₆ alkyl, C₃-C₆ cycloalkyl, or —C(═O)—(C₁-C₆ alkyl);

R⁵ is selected from the group consisting of H, C₁-C₈ alkyl, C₃-C₈ cycloalkyl, C₄-C₈ cycloalkenyl, C₂-C₈ alkenyl, C₂-C₈ alkynyl, and —C(═O)—(C₁-C₄ alkyl); wherein the C₁-C₈ alkyl, C₃-C₈ cycloalkyl, C₄-C₈ cycloalkenyl, C₂-C₈ alkenyl, or C₂-C₈ alkynyl is optionally substituted with one or more fluoro, phenyl, or OR^{1a}; or wherein R^{4a} and R⁵ are taken together with the atoms to which they are attached to form a 5-8 membered heterocycl optionally substituted with halogen, N(R^{3a})(R^{3b}), OR^{1a}, or optionally substituted C₁-C₃ alkyl; wherein when a carbon atom of the heterocycl has two C₁-C₃ alkyl substituents attached thereto, the two C₁-C₃ alkyl substituents together with the carbon atom to which they are attached can form a 3-8 membered cycloalkyl or heterocycl comprising one or more heteroatoms selected from O, N and S;

R⁶ is selected from the group consisting of H, F, Cl, Br, and C₁-C₆ alkyl;

R⁷ is selected from the group consisting of CN, tetrazolyl, CH₂OR^{7a}, CFHOR^{7a}, CF₂OR^{7a}, CO₂R^{7a}, CON(R^{7b})₂, and C(═NH)—N(R^{7b})₂; wherein R^{7a} is H, C₁-C₆ alkyl or C₃-C₆ cycloalkyl; and each R^{7b} is independently H, OH, C₁-C₆ alkyl, or C₃-C₆ cycloalkyl;

L is selected from the group consisting of a bond, —(CH₂)₁₋₄—, —C₄-C₈ cycloalkenyl-, —(CH₂)ₙN(R^{La})(CH₂)ₙ—, -cycloalkyl-N(R^{La})—, —(CH₂)ₙO—, -aryl-, -heterocycl-, and -heteroaryl-; wherein L is optionally substituted with one or more halo or C₁-C₄ alkyl; wherein R^{La} is H or C₁-C₈ alkyl; and each n is independently 0 to 4;

R⁸ is selected from the group consisting of C₃-C₈ cycloalkyl-N(R^{8a})(R^{8b}), aryl-C₃-C₈ cycloalkyl-N(R^{8a})(R^{8b}), N(R^{8a})—C₃-C₈ cycloalkyl-aryl, C₄-C₈ cycloalkenyl, OR^{8b}, and N(R^{8a})(R^{8b}); wherein R⁸ is optionally substituted with F or C₁-C₆ alkyl; R^{8a} is H or C₁-C₈ alkyl; and R^{8b} is H or C₁-C₈ alkyl.

Embodiment 3. The compound of any of embodiments 1 and 2, wherein Q¹ is N.

Embodiment 4. The compound of any of embodiments 1 and 2, wherein Q¹ is CR⁶.

Embodiment 5. The compound of any one of the preceding embodiments, wherein R¹ is selected from the group consisting of H, F, NO₂, OH, N(R^{1a})₂, and C₁-C₃ alkyl.

Embodiment 6. The compound of any one of the preceding embodiments, wherein R¹ is selected from the group consisting of H, NH₂, NHCH₂Ph, NO₂, and CH₃.

Embodiment 7. The compound of any one of the preceding embodiments, wherein R¹ is NH₂.

Embodiment 8. The compound of any one of the preceding embodiments, wherein R² is F.

Embodiment 9. The compound of any one of the preceding embodiments, wherein R² is NHCH₂CH₃.

Embodiment 10. The compound of any one of the preceding embodiments, wherein R³ is selected from the group consisting of N(R^{3a})(R^{3b}), C₁₋₃ alkyl-N(R^{3a})(R^{3b}), OR^{3b}, CH₂N(R^{3a})CH₂CN, N(R^{3a})CH₂CN, aryl optionally substituted with R^{3b}, and C₃-C₈ cycloalkyl optionally substituted with NR¹⁰R¹¹.

Embodiment 11. The compound of any one of the preceding embodiments, wherein R³ is N(R^{3a})(R^{3b}).

Embodiment 12. The compound of any one of the preceding embodiments, wherein R³ is NH(R^{3b}).

Embodiment 13. The compound of any one of the preceding embodiments, wherein wherein R³ is NH(R^{3b}) and R^{3b} is selected from the group consisting of C₃-C₈ cycloalkyl, C₄-C₈ heterocycloalkyl, C₃-C₈ cycloalkyl substituted with NR¹⁰R¹¹, C₃-C₈ cycloalkyl substituted with heteroaryl, C₁-C₈ alkyl substituted with phenyl, C₁-C₈ alkyl substituted with NR¹⁰R¹¹, indanyl, and phenyl optionally substituted with C₃-C₆ cycloalkyl optionally substituted with NR¹⁰R¹¹.

Embodiment 14. The compound of any one of the preceding embodiments, wherein R³ is NH(R^{3b}) and R^{3b} is selected from the group consisting of C₃-C₈ cycloalkyl substituted with NHR¹¹, C₁-C₈ alkyl substituted with NHR¹¹, and phenyl optionally substituted with C₃-C₆ cycloalkyl optionally substituted with NHR¹¹, wherein R¹¹ is selected from the group consisting of H and C₃-C₈ cycloalkyl substituted with an optionally substituted phenyl.

Embodiment 15. The compound of any one of the preceding embodiments, wherein R³ is NH(R^{3b}) and R^{3b} is C₃-C₈ cycloalkyl or C₄-C₈ heterocycloalkyl.

Embodiment 16. The compound of any one of the preceding embodiments, R³ is selected from the group consisting of

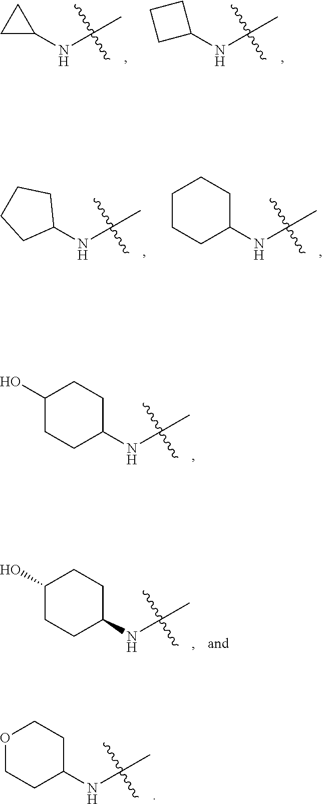

Embodiment 17. The compound of any one of the preceding embodiments, wherein R³ is N(R^{3a})(R^{3b}) and R^{3b} is C₃-C₈ cycloalkyl optionally substituted with an optionally substituted phenyl.

Embodiment 18. The compound of any one of the preceding embodiments, wherein R³ is NH(R^{3b}) and R^{3b} is cyclopropyl substituted with phenyl or cyclobutyl substituted with phenyl.

Embodiment 19. The compound of any one of the preceding embodiments, wherein R³ is selected from the group consisting of:

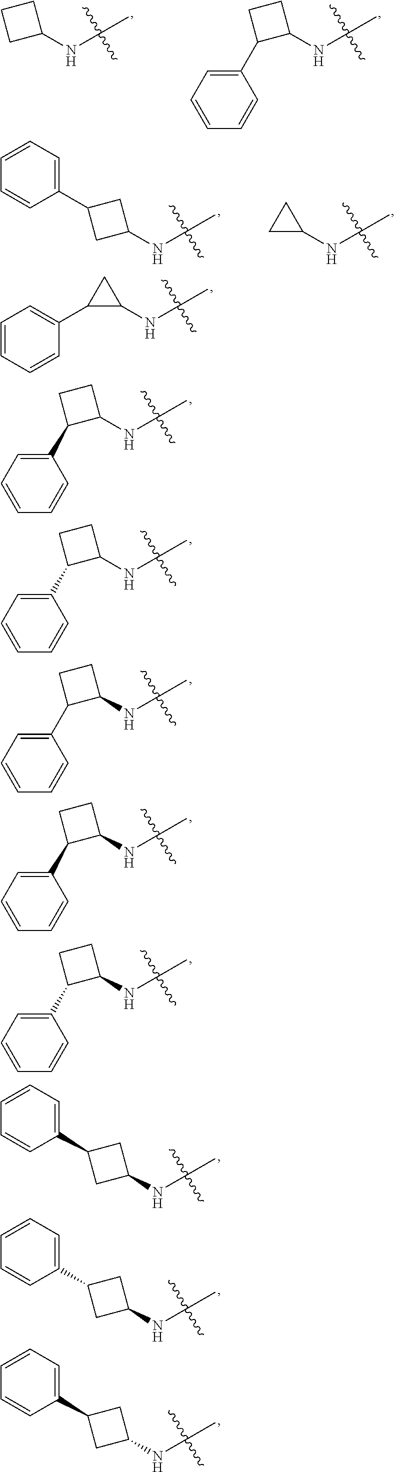

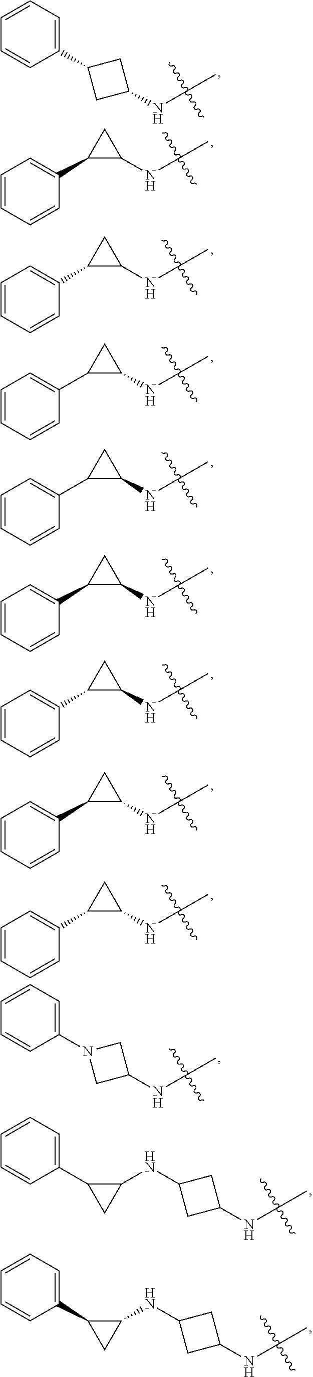

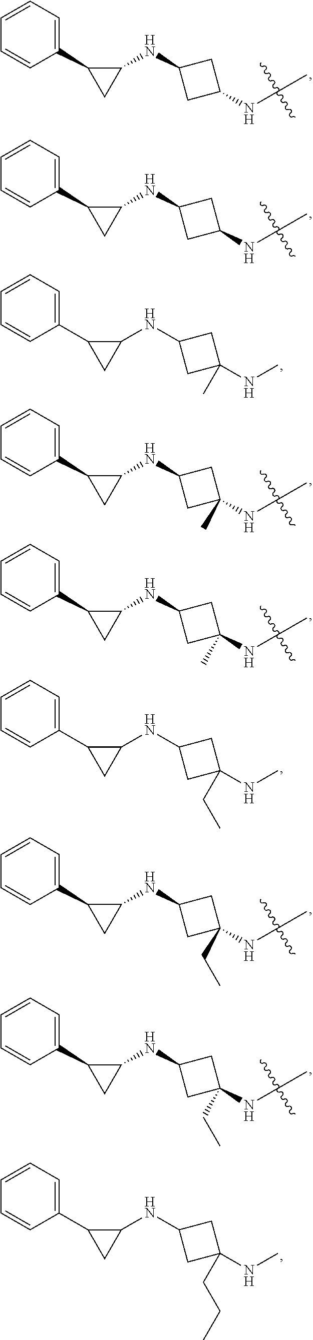

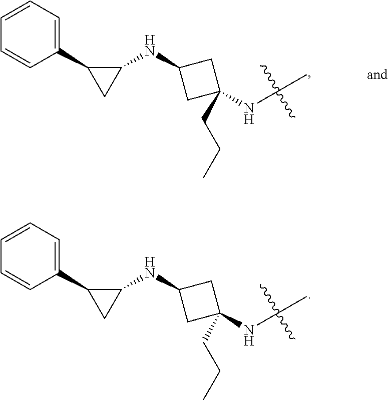 [SMILES: *N[C@]1(CCC)C[C@@H](N[C@@H]2C[C@H]2c2ccccc2)C1 ; *N[C@]1(CCC)C[C@H](N[C@@H]2C[C@H]2c2ccccc2)C1]

Embodiment 20. The compound of any one of the preceding embodiments, wherein R³ is NH(R^{3a})(R^{3b}) and R^{3b} is C₃-C₈ cycloalkyl optionally substituted with one or more C₁-C₄ alkyl and phenyl optionally substituted with C₁-C₄ alkyl.

Embodiment 21. The compound of any one of the preceding embodiments, wherein R³ is NH(R^{3b}) and R^{3b} is cyclopropyl substituted with one or more methyl and one or more phenyl.

Embodiment 22. The compound of any one of the preceding embodiments, wherein R³ is NH(R^{3b}) and R^{3b} is C₃-C₈ cycloalkyl substituted with NR¹⁰R¹¹.

Embodiment 23. The compound of any one of the preceding embodiments, wherein R³ is NH(R^{3b}) and R^{3b} is C₃-C₈ cycloalkyl substituted with NHR¹¹ and R¹¹ is C₃-C₈ cycloalkyl substituted with an optionally substituted phenyl.

Embodiment 24. The compound of any one of the preceding embodiments, wherein R³ is selected from the group consisting of

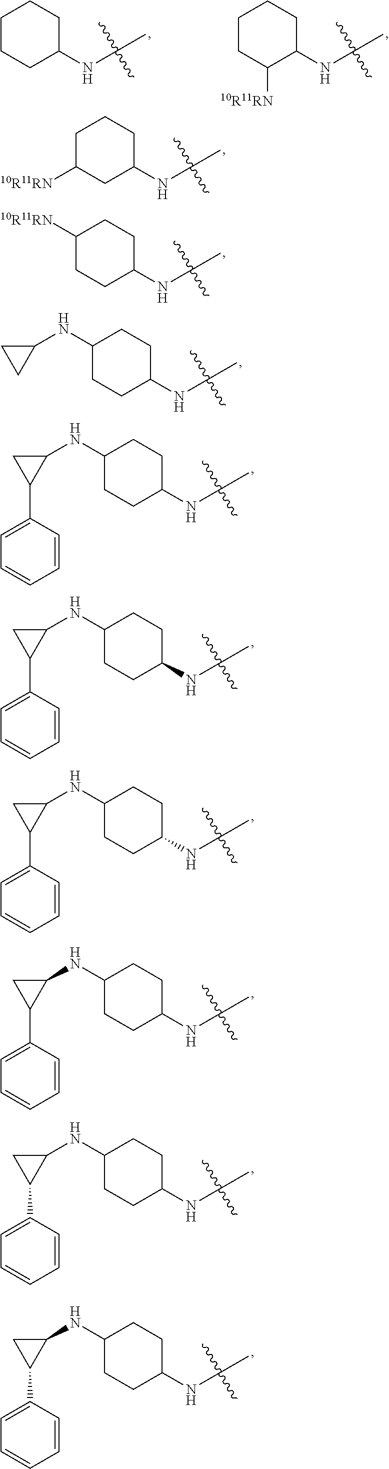

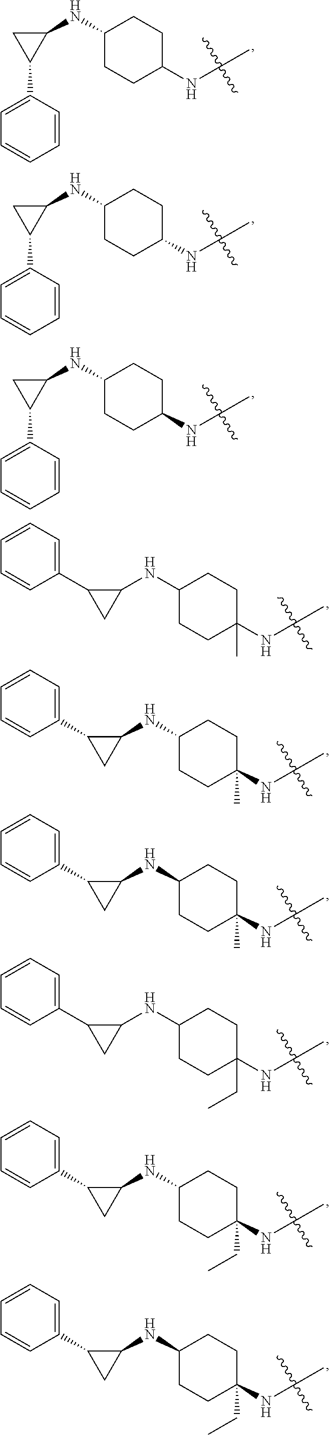

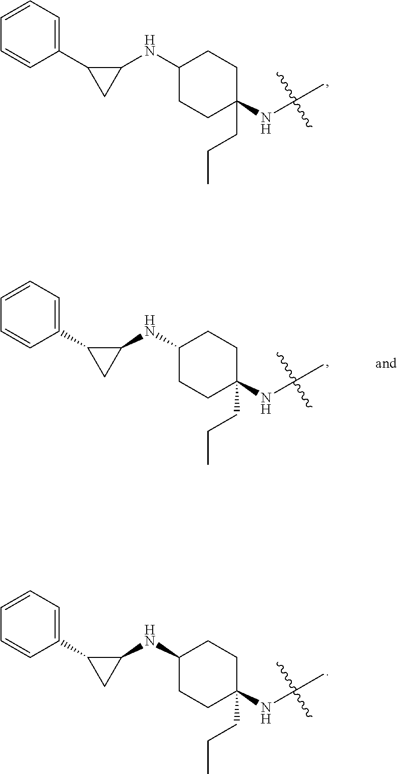

Embodiment 25. The compound of any one of the preceding embodiments, wherein R³ is NH(R^{3b}) and R^{3b} is C₃-C₈ cycloalkyl substituted with heteroaryl.

Embodiment 26. The compound of any one of the preceding embodiments, wherein R³ is selected from the group consisting of:

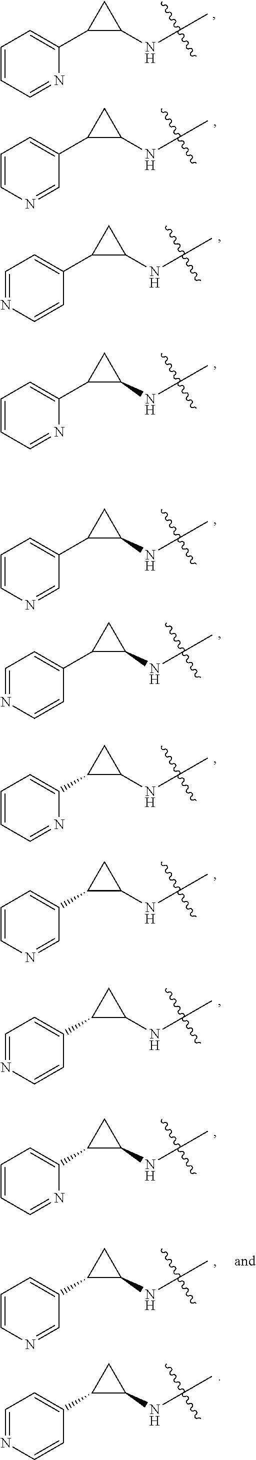

Embodiment 27. The compound of any one of the preceding embodiments, wherein R³ is NH(R^{3b}) and R^{3b} is C₁-C₈ alkyl substituted with phenyl.

Embodiment 28. The compound of any one of the preceding embodiments, wherein R³ is selected from the group consisting of:

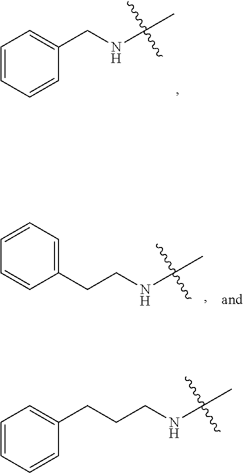 [SMILES: *NCc1ccccc1 ; *NCCc1ccccc1 ; *NCCCc1ccccc1]

Embodiment 29. The compound of any one of the preceding embodiments, wherein R³ is NH(R^{3b}) and R^{3b} is C₁-C₈ alkyl substituted with NR¹⁰R¹¹.

Embodiment 30. The compound of any one of the preceding embodiments, wherein R³ is NH(R^{3b}) and R^{3b} is C₁-C₈ alkyl substituted with NHR¹¹ and R¹¹ is C₃-C₈ cycloalkyl substituted with an optionally substituted phenyl.

Embodiment 31. The compound of any one of the preceding embodiments, wherein R³ is selected from the group consisting of

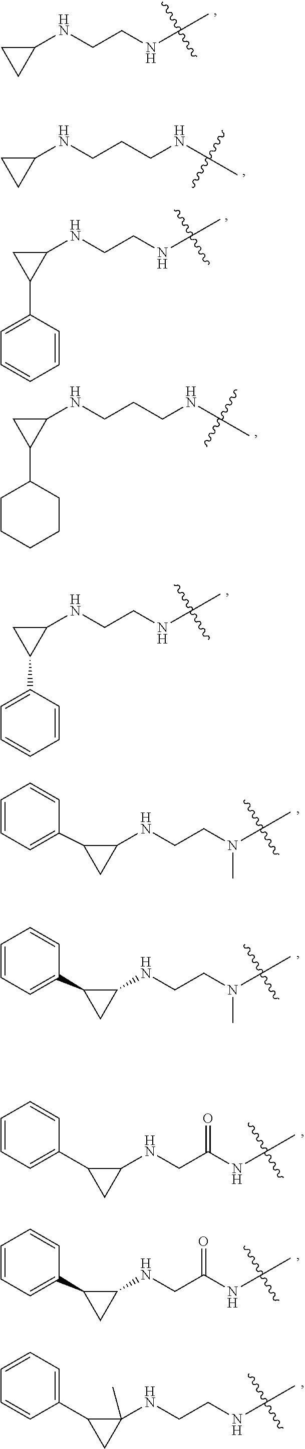

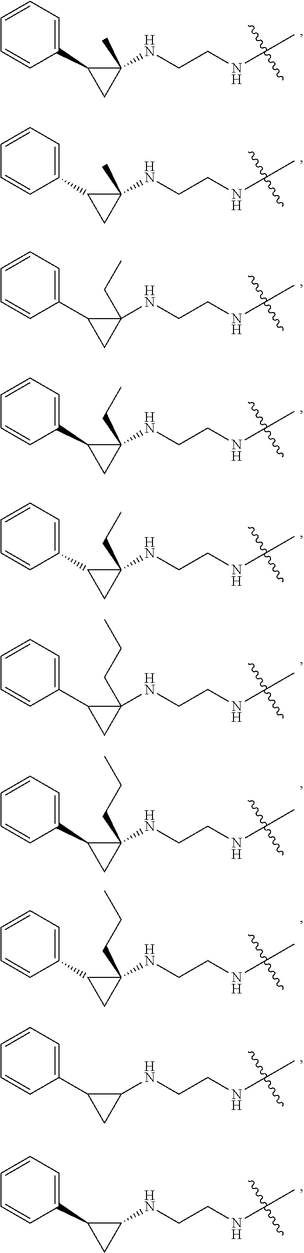

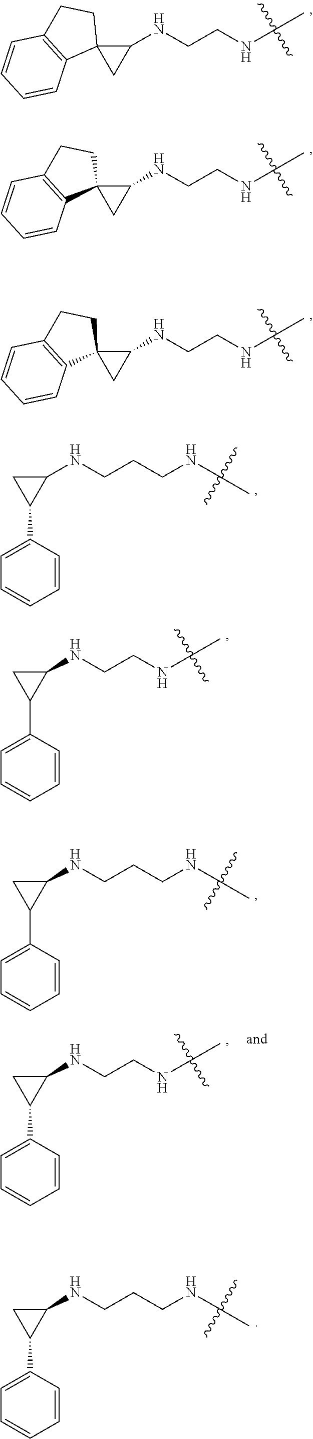

Embodiment 32. The compound of any one of the preceding embodiments, wherein R³ is NH(R^{3b}) and R^{3b} is indanyl or phenyl, wherein the phenyl is optionally substituted with C₃-C₆ cycloalkyl optionally substituted with NR¹⁰R¹¹.

Embodiment 33. The compound of any one of the preceding embodiments, wherein R³ is selected from the group consisting of

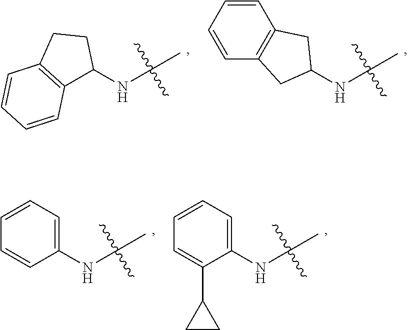

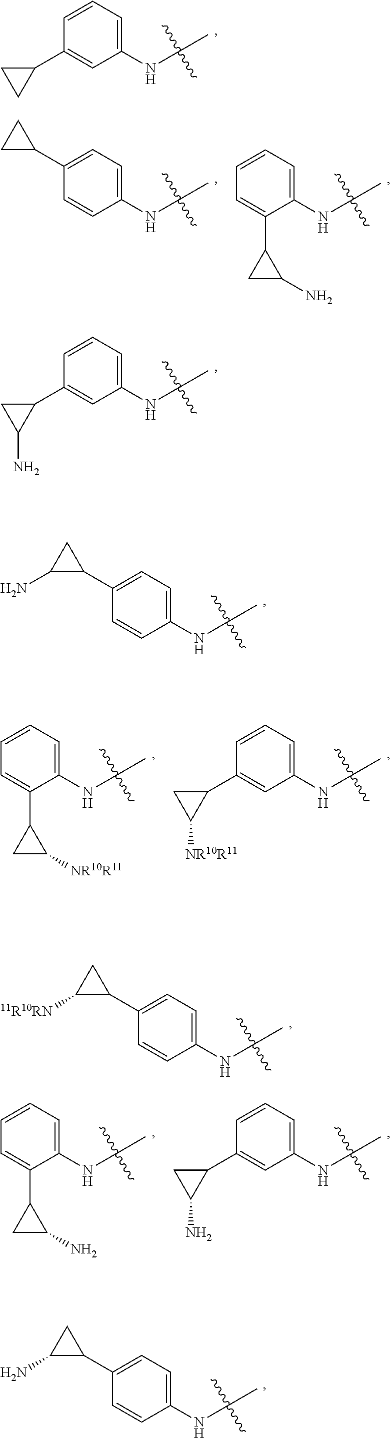

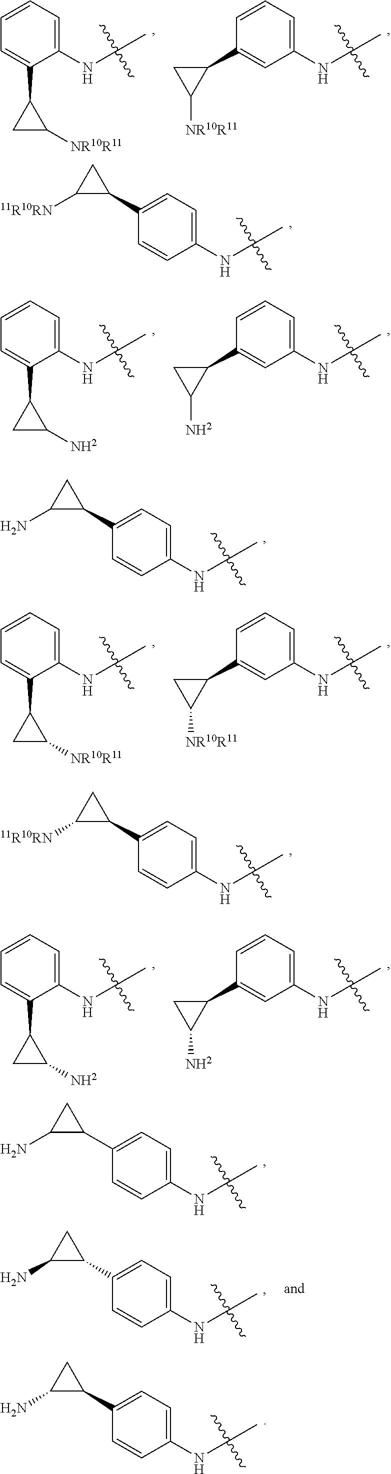

Embodiment 34. The compound of any one of the preceding embodiments, wherein R³ is N(R^{3a})(R^{3b}) wherein R^{3a} and R^{3b} are taken together with the N to which they are attached to form a 3-6 membered heterocycl substituted with one or more NR¹⁰R¹¹.

Embodiment 35. The compound of any one of the preceding embodiments, wherein R³ is N(R^{3a})(R^{3b}) wherein R^{3a} and R^{3b} are taken together with the N to which they are attached to form a 6 membered heterocycl substituted with NR¹⁰R¹¹ wherein R¹⁰ is H and R¹¹ is C₃-C₈ cycloalkyl substituted with an optionally substituted phenyl.

Embodiment 36. The compound of any one of the preceding embodiments, wherein R³ is selected from the group consisting of:

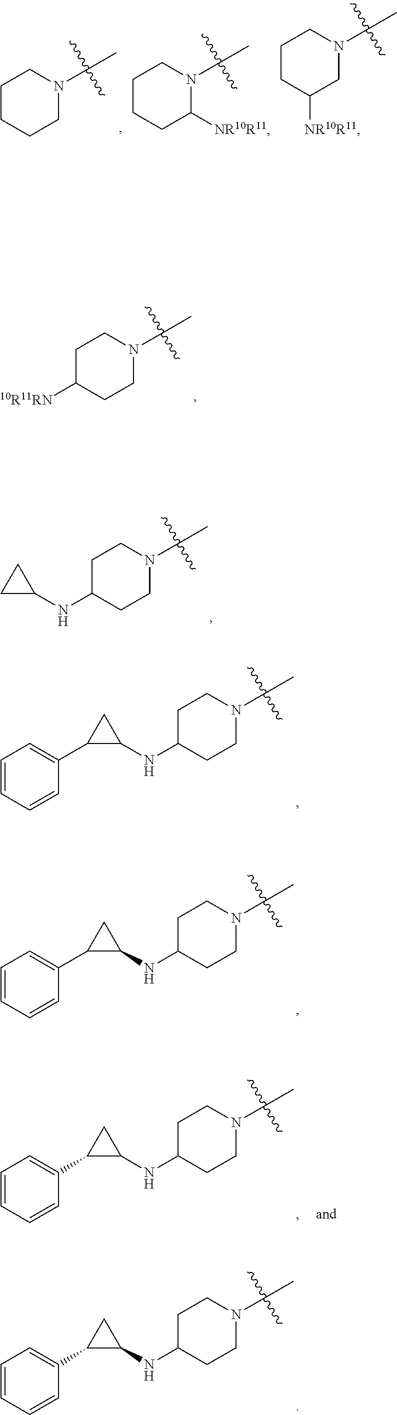

Embodiment 37. The compound of any one of the preceding embodiments, wherein R³ is NH(R^{3b}) and R^{3b} is C₃-C₈ cycloalkyl substituted with two C₁-C₄ alkyl substitutents and optionally further substituted with phenyl; and wherein the two C₁-C₄ alkyl substituents together with the carbon atom(s) to which they are attached form a C₃-C₈ cycloalkyl or C₆-C₁₆ aryl.

Embodiment 38. The compound of any one of the preceding embodiments, wherein R³ is selected from the group consisting of:

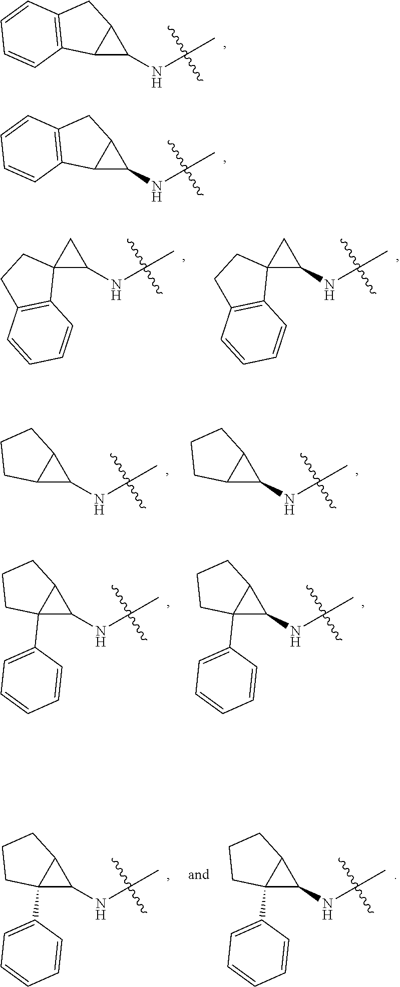

Embodiment 39. The compound of any one of the preceding embodiments, wherein R³ is C₁-C₄ alkyl-N(R^{3a})(R^{3b}).

Embodiment 40. The compound of any one of the preceding embodiments, wherein R³ is C₁-C₄ alkyl-NH(R^{3b}) and R^{3b} is C₃-C₈ cycloalkyl substituted with phenyl.

Embodiment 41. The compound of any one of the preceding embodiments, wherein R³ is selected from the group consisting of

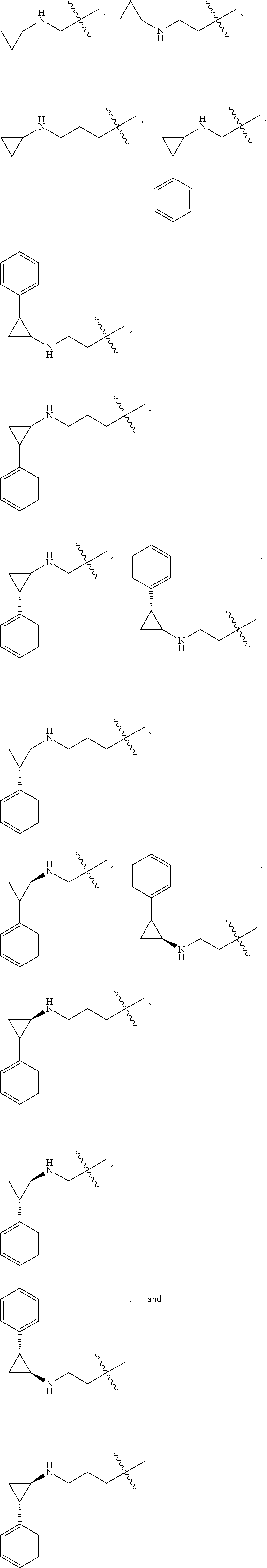

Embodiment 42. The compound of any one of the preceding embodiments, wherein R³ is OR^{3b}.

Embodiment 43. The compound of any one of the preceding embodiments, wherein R³ is selected from the group consisting of:

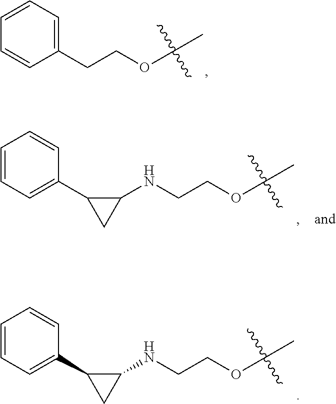 [SMILES: *OCCc1ccccc1 ; *OCCNC1CC1c1ccccc1 ; *OCCN[C@@H]1C[C@H]1c1ccccc1]

Embodiment 44. The compound of any one of the preceding embodiments, wherein R³ is selected from the group consisting of CH₂N(R^{3a})CH₂CN and N(R^{3a})CH₂CN.

Embodiment 45. The compound of any one of the preceding embodiments, wherein R³ is selected from the group consisting of CH₂NHCH₂CN, CH₂N(CH₃)CH₂CN, NHCH₂CN, and N(CH₃)CH₂CN.

Embodiment 46. The compound of any one of the preceding embodiments, wherein R³ is aryl optionally substituted with R^{3b}.

Embodiment 47. The compound of any one of the preceding embodiments, wherein R³ is aryl optionally substituted with R^{3b} and R^{3b} is C₃-C₆ cycloalkyl optionally substituted with NR¹⁰R¹¹.

Embodiment 48. The compound of any one of the preceding embodiments, wherein R³ is selected from the group consisting of:

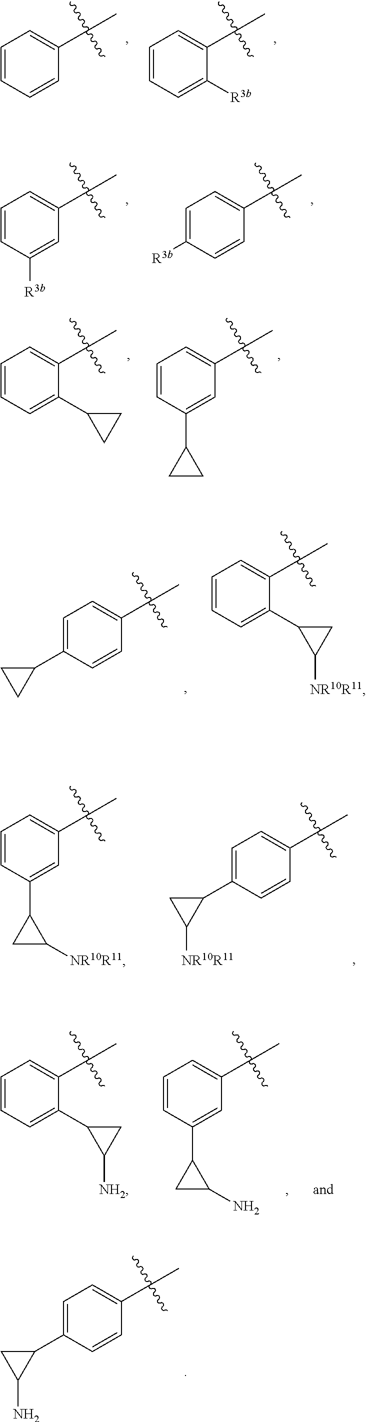

Embodiment 49. The compound of any one of the preceding embodiments, wherein R³ is C₃-C₈ cycloalkyl optionally substituted with NR¹⁰R¹¹.

Embodiment 50. The compound of any one of the preceding embodiments, R³ is selected from the group consisting of

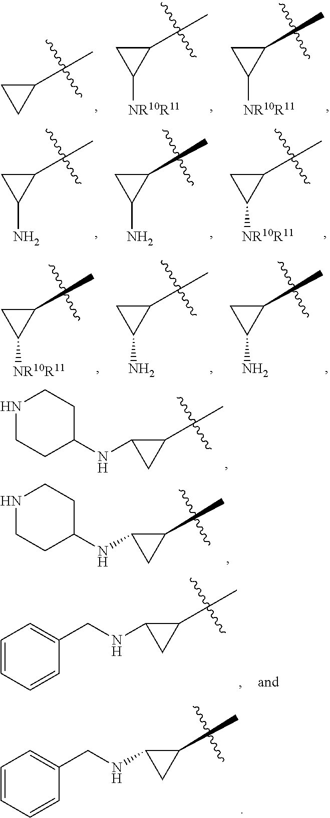

Embodiment 50a. The compound of any one of the preceding embodiments, wherein R³ is —N(R^{3a})(R^{3b}).

Embodiment 50b. The compound of embodiment 50a, wherein R³ is N(R^{3a})(R^{3b}), N(R^{3a})(R^{3b}) is further defined by:

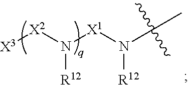

wherein each R¹² is independently selected from the group consisting of H and C₁-C₈ alkyl; wherein X¹ is selected from the group consisting of C₃-C₈ cycloalkyl, C₁-C₈ alkyl,

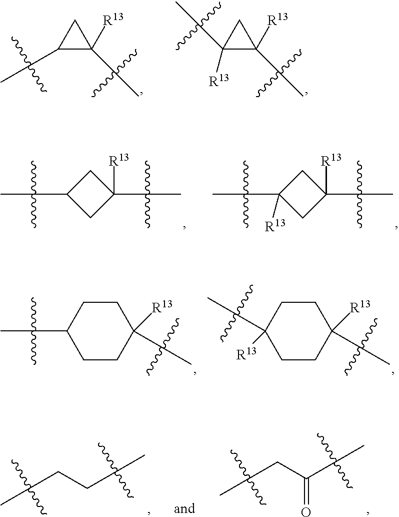

optionally wherein X¹ is substituted with one or more halogen, C₃-C₆ cycloalkyl or C₁₋₄ alkyl; wherein X² is selected from the group consisting of C₃-C₈ cycloalkyl, C₁-C₈ alkyl,

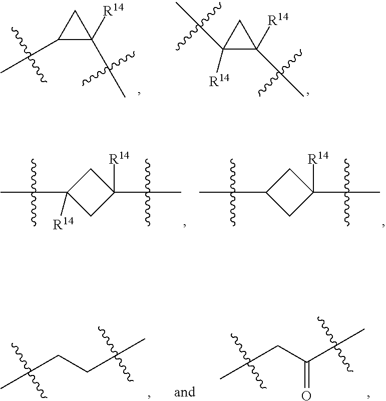

optionally wherein X² is substituted with one or more halogen, C₃-C₆ cycloalkyl or C₁₋₄ alkyl; wherein q is 0 or 1; wherein in X³ is selected from the group consisting of H, phenyl, phenyl substituted with C₁-C₄ alkyl, C₃-C₈ cycloalkyl, and C₁-C₈ alkyl, optionally wherein X³ is substituted with one or more halogen, C₃-C₆ cycloalkyl or C₁₋₄ alkyl; wherein R¹³ is independently selected from the group consisting of H, C₃-C₈ cycloalkyl, and C₁-C₈ alkyl; and wherein R¹⁴ is independently selected from the group consisting of H, C₃-C₈ cycloalkyl, and C₁-C₈ alkyl.

Embodiment 50c. The compound of embodiment 50b, wherein X¹ is selected from the group consisting of

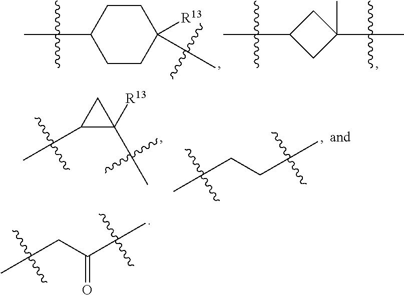

Embodiment 50d. The compound of embodiment 50b or 50c, wherein X² is selected from the group consisting of

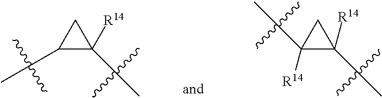

Embodiment 50e. The compound of any one of embodiments 50b to 50d, wherein X³ is selected from the group consisting of H, phenyl, and phenyl substituted with C₁-C₄ alkyl.

Embodiment 50f. The compound of any one of embodiments 50b to 50e, wherein q is 1; X¹ is selected from the group consisting of

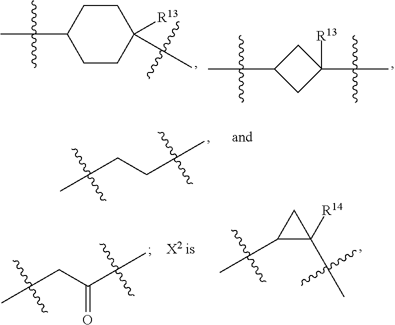

X³ is selected from the group consisting of phenyl and phenyl substituted with C₁-C₄ alkyl; R¹² is H; R¹³ is H or C₁-C₈ alkyl; and R¹⁴ is H or C₁-C₈ alkyl.

Embodiment 50g. The compound of any one of embodiments 50b to 50e, q is 0; X¹ is selected from the group consisting of

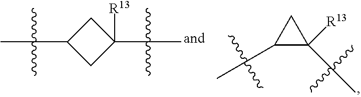

X³ is selected from the group consisting of phenyl and phenyl substituted with C₁-C₄ alkyl; R¹² is H; and R¹³ is H or C₁-C₈ alkyl.

Embodiment 50h. The compound of any one of embodiments 50b to 50g, wherein R¹² is independently selected from the group consisting of H, methyl, ethyl, n-propyl, isopropyl, and n-butyl.

Embodiment 50i. The compound of any one of embodiments 50b to 50h, wherein R¹³ is independently selected from the group consisting of H, methyl, ethyl, n-propyl, isopropyl, and n-butyl.

Embodiment 50j. The compound of any one of embodiments 50b to 50i, wherein R¹⁴ is independently selected from the group consisting of H, methyl, ethyl, n-propyl, isopropyl, and n-butyl.

Embodiment 50k. The compound of any one of embodiments 50b to 50f and 50h to 50j, wherein q is 1.

Embodiment 50l. The compound of any one of embodiments 50b to 50e and 50g to 50j, wherein q is 0.

Embodiment 51. The compound of any one of the preceding embodiments, wherein R⁴ is selected from the group consisting of H, Cl, and F.

Embodiment 52. The compound of any one of the preceding embodiments, wherein R⁴ is H.

Embodiment 53. The compound of any one of the preceding embodiments, wherein R⁵ is selected from the group consisting of C₁-C₈ alkyl and C₃-C₈ cycloalkyl.

Embodiment 54. The compound of any one of the preceding embodiments, wherein R⁵ is selected from the group consisting of CH₃, CH₂CH₃, CH₂CH₂CH₃, CH(CH₃)₂, CH(CH₂CH₃)₂, and cyclopropyl.

Embodiment 55. The compound of any one of the preceding embodiments, wherein R⁵ is CH₂CH₃.

Embodiment 56. The compound of any one of the preceding embodiments, wherein R⁵ is C₁-C₈ alkyl optionally substituted with OR^{1a}.

Embodiment 57. The compound of any one of the preceding embodiments, wherein R⁵ is CH₂CH₂OH.

Embodiment 58. The compound of any one of the preceding embodiments, wherein R⁶ is H.

Embodiment 59. The compound of any one of the preceding embodiments, wherein R⁷ is selected from the group consisting of CN, tetrazolyl, CH₂OR^{7a}, CO₂R^{7a}, CON(R^{7b})₂, and C(═NH)—N(R^{7b})₂.

Embodiment 60. The compound of any one of the preceding embodiments, wherein R⁷ is selected from the group consisting of CN, CH₂OH, CO₂H, CO₂CH₃, CO₂CH₂CH₃, CONH₂, CONHOH, CONHCH₃, CON(CH₃)₂, and C(═NH)—NHOH.

Embodiment 61. The compound of any one of the preceding embodiments, wherein R⁷ is selected from the group consisting of CN and CO₂H.

Embodiment 62. The compound of any one of the preceding embodiments, wherein L is selected from the group consisting of a bond, —(CH₂)₁₋₄—, —(CH₂)ₙN(R^{La})(CH₂)ₙ—, and -cycloalkyl-N(R^{La})—.

Embodiment 63. The compound of any one of the preceding embodiments, wherein L is selected from the group consisting of a bond, —CH₂—, —N(R^{La})CH₂—, —CH₂N(R^{La})—, -cyclopropyl-N(R^{La})—, and -cyclobutyl-N(R^{La})—.

Embodiment 64. The compound of any one of the preceding embodiments, wherein R⁸ is selected from the group consisting of C₃-C₈ cycloalkyl-N(R^{8a})(R^{8b}), N(R^{8a})—C₃-C₈ cycloalkyl-aryl, and aryl-C₃-C₈ cycloalkyl-N(R^{8a})(R^{8b}).

Embodiment 65. The compound of any one of the preceding embodiments, wherein R⁸ is selected from the group consisting of cyclopropyl-NH₂, cyclobutyl-NH₂, phenyl-cyclopropyl-NH₂, phenyl-cyclobutyl-NH₂, NH-cyclopropyl-phenyl, and NH-cyclobutyl-phenyl.

Embodiment 66. The compound of any one of the preceding embodiments, wherein the compound is of Formulae (Ia), (Ib), (Ic), (Id), (Ie), (If), (Ig), (Ih), (Ii), (Ij), or (Ik):

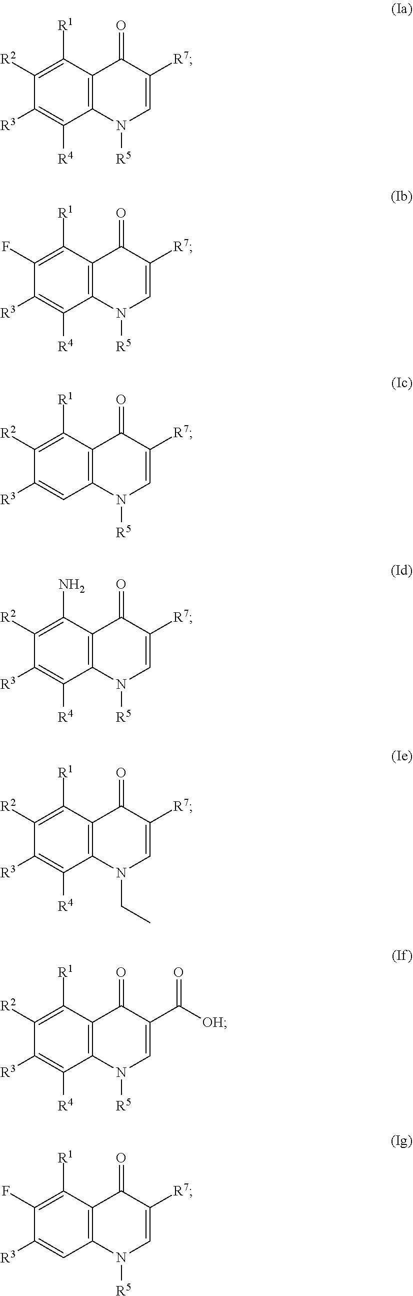

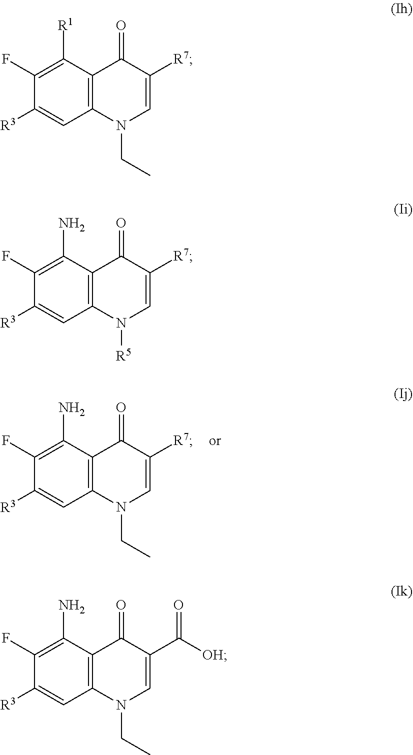

or a pharmaceutically acceptable salt or tautomer thereof, wherein R¹, R², R³, R⁴, R⁵, and R⁷ are as described herein.

Embodiment 67. The compound of any one of the preceding embodiments, wherein the compound is of Formulae (IIIa), (IIIb), (IIIc), or (IIId):

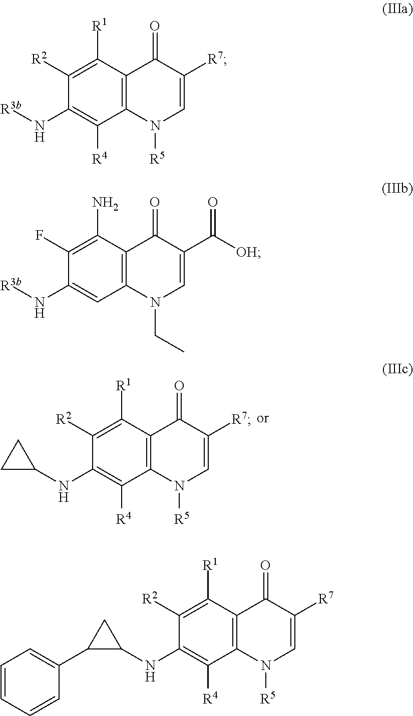

(IIId); or a pharmaceutically acceptable salt or tautomer thereof, wherein R¹, R², R^{3b}, R⁴, R⁵ and R⁷ are as described herein.

Embodiment 68. The compound of any one of the preceding embodiments, wherein the compound is of Formulae (IVa), (IVb), (IVc), or (IVd):

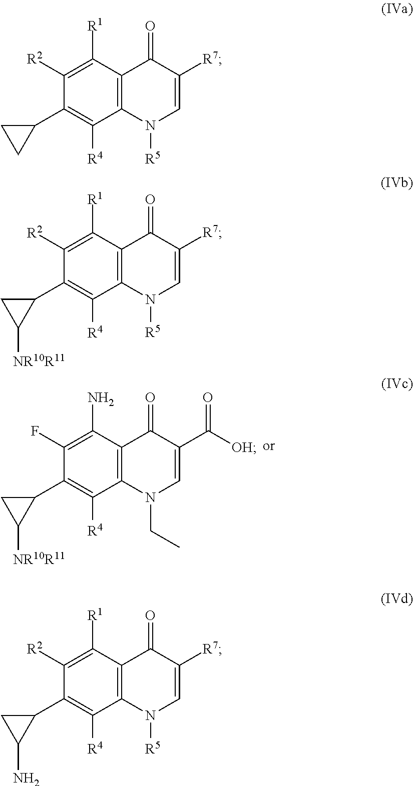

or a pharmaceutically acceptable salt or tautomer thereof, wherein R¹, R², R⁴, R⁵, R⁷, R¹⁰ and R¹¹ are as described herein.

Embodiment 69. The compound of any one of the preceding embodiments, wherein the compound is of Formulae (Va), (Vb), (Vc), or (Vd):

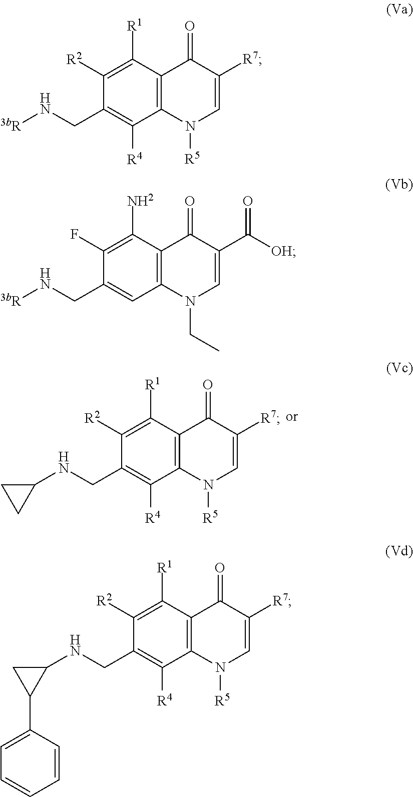

or a pharmaceutically acceptable salt or tautomer thereof, wherein R¹, R², R^{3b}, R⁴, R⁵ and R⁷ are as described herein.

Embodiment 70. The compound of any one of the preceding embodiments, wherein the compound is of Formulae (VIa), (VIb), (VIc), (VId), (VIe), (VIf), (VIg), (VIh), (VII), (VIj), or

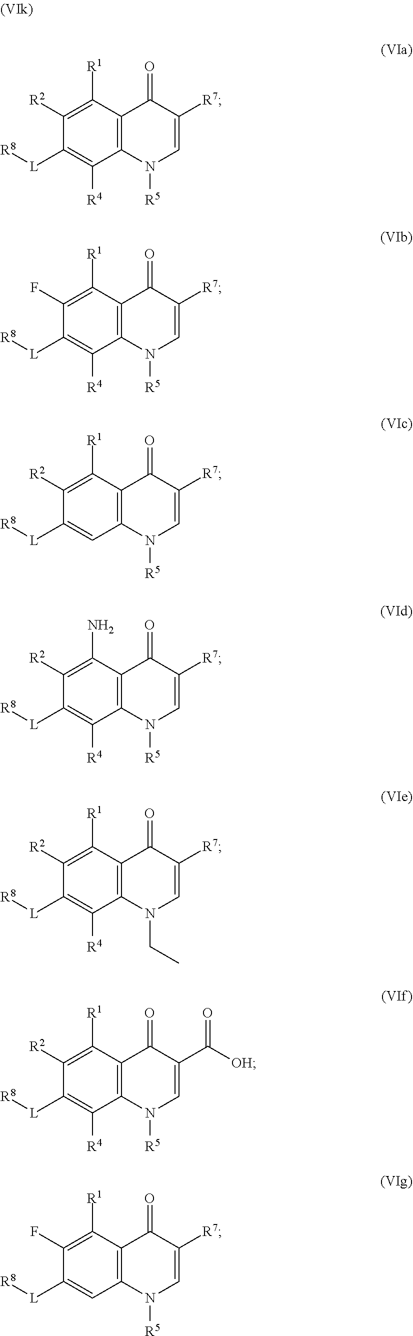

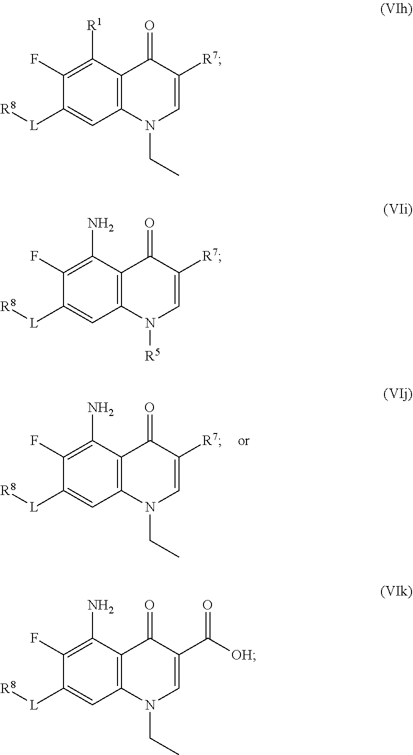

or a pharmaceutically acceptable salt or tautomer thereof, wherein R¹, R², R⁴, R⁵, R⁷, L and R⁸ are as described herein.

Embodiment 71. The compound of any one of the preceding embodiments, wherein the compound is of Formulae (VIIa) or (VIIb):

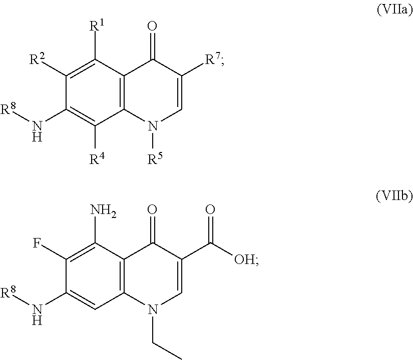

or a pharmaceutically acceptable salt or tautomer thereof, wherein R¹, R², R⁴, R⁵, R⁷ and R⁸ are as described herein.

Embodiment 72. The compound of any one of the preceding embodiments, wherein the compound is of Formulae (VIIIa) or (VIIIb):

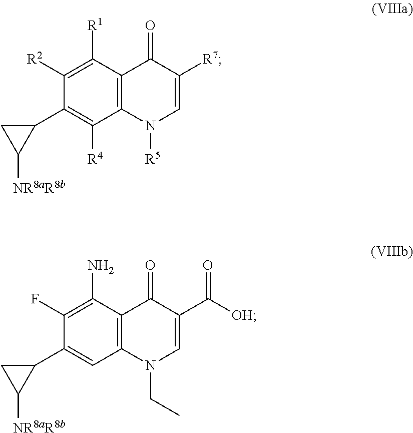

or a pharmaceutically acceptable salt or tautomer thereof, wherein R¹, R², R⁴, R⁵, R⁷, R^{8a} and R^{b8} are as described herein.

Embodiment 73. The compound of any one of the preceding embodiments, wherein the compound is of Formulae (IXa) or (IXb):

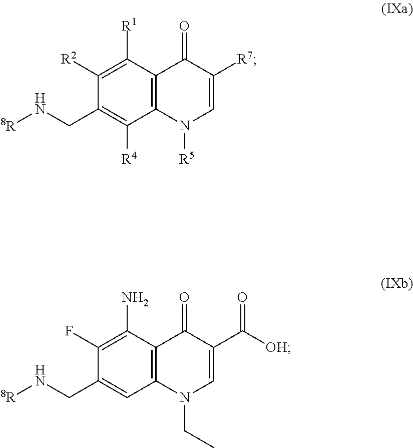

or a pharmaceutically acceptable salt or tautomer thereof, wherein R¹, R², R⁴, R⁵, R⁷ and R⁸ are as described herein.

Embodiment 74. A compound selected Table A or a pharmaceutically acceptable salt or tautomer thereof.

Embodiment 75. The compound of any one of the preceding embodiments, wherein the compound has a measured IC₅₀ value of about 0.1 nM to about 10 μM, about 0.5 nM to about 5 μM, about 1 nM to about 1 μM, about 2 nM to about 900 nM, about 3 nM to about 800 nM, about 4 nM to about 700 nM, about 5 nM to about 600 nM, about 10 nM to about 500 nM, about 20 nM to about 400 nM, about 30 nM to about 300 nM, about 40 nM to about 250 nM, about 50 nM to about 200 nM, about 60 nM to about 150 nM, about 70 nM to about 100 nM, or about 80 nM to about 90 nM against GSK3alpha and/or GSK3beta.

Embodiment 76. The compound of any one of the preceding embodiments, wherein the compound has a measured IC₅₀ value of about 0.1 nM or less, about 1 nM or less, about 2 nM or less, about 3 nM or less, about 4 nM or less, about 5 nM or less, about 10 nM or less, about 25 nM or less, about 50 nM or less, about 100 nM or less, about 250 nM or less, about 500 nM or less, about 1 μM or less, or about 10 μM or less against GSK3alpha and GSK3beta.

Embodiment 77. The compound of any one of the preceding embodiments, wherein the compound has a measured inhibitory activity value of about 0% to about 100%, about 1% to about 95%, about 2% to about 90%, about 3% to about 85%, about 4% to about 80%, about 5% to about 75%, about 10% to about 70%, about 15% to about 65%, about 20% to about 60%, about 25% to about 55%, about 30% to about 50%, about 35% to about 45%, or about 40% to about 45% against LSD-1 or LSD-2.

Embodiment 78. The compound of any one of the preceding embodiments, wherein the compound has a measured inhibitory activity value of about 1% or greater, about 2% or greater, about 3% or greater, about 4% or greater, about 5% or greater, about 10% or greater, about 15% or greater, about 20% or greater, about 25% or greater, about 30% or greater, about 40% or greater, about 50% or greater, about 60% or greater, about 70% or greater, about 80% or greater, about 90% or greater, or about 95% or greater against LSD-1 or LSD-2.

Embodiment 79. The compound of any one of the preceding embodiments, wherein the compound has a measured IC₅₀ value of about 0.1 nM to about 10 μM, about 0.5 nM to about 5 μM, about 1 nM to about 1 μM, about 2 nM to about 900 nM, about 3 nM to about 800 nM, about 4 nM to about 700 nM, about 5 nM to about 600 nM, about 10 nM to about 500 nM, about 20 nM to about 400 nM, about 30 nM to about 300 nM, about 40 nM to about 250 nM, about 50 nM to about 200 nM, about 60 nM to about 150 nM, about 70 nM to about 100 nM, or about 80 nM to about 90 nM against Foxo-1.

Embodiment 80. The compound of any one of the preceding embodiments, wherein the compound has a measured IC₅₀ value of about 0.1 nM or less, about 1 nM or less, about 2 nM or less, about 3 nM or less, about 4 nM or less, about 5 nM or less, about 10 nM or less, about 25 nM or less, about 50 nM or less, about 100 nM or less, about 250 nM or less, about 500 nM or less, about 1 μM or less, or about 10 μM or less against Foxo-1.

Embodiment 81. The compound of any one of the preceding embodiments, wherein the compound has a measured EC value of about 0.1 nM to about 100 μM, about 0.5 nM to about 10 μM, about 1 nM to about 1 μM, about 2 nM to about 900 nM, about 3 nM to about 800 nM, about 4 nM to about 700 nM, about 5 nM to about 600 nM, about 10 nM to about 500 nM, about 20 nM to about 400 nM, about 30 nM to about 300 nM, about 40 nM to about 250 nM, about 50 nM to about 200 nM, about 60 nM to about 150 nM, about 70 nM to about 100 nM, or about 80 nM to about 90 nM for expansion of Lgr5+ cells.

Embodiment 82. The compound of any one of the preceding embodiments, wherein the compound has a measured EC value of about 0.1 nM or less, about 1 nM or less, about 2 nM or less, about 3 nM or less, about 4 nM or less, about 5 nM or less, about 10 nM or less, about 25 nM or less, about 50 nM or less, about 100 nM or less, about 250 nM or less, about 500 nM or less, about 1 μM or less, about 10 μM, or about 100 μM or less for expansion of Lgr5+ cells.

Embodiment 83. The compound of any one of the preceding embodiments, wherein the compound increases the percentage of Lgr5+ cells to value of about 0% to about 100%, about 1% to about 95%, about 2% to about 90%, about 3% to about 85%, about 4% to about 80%, about 5% to about 75%, about 10% to about 70%, about 15% to about 65%, about 20% to about 60%, about 25% to about 55%, about 30% to about 50%, about 35% to about 45%, or about 40% to about 45%

Embodiment 84. The compound of any one of the preceding embodiments, wherein the compound increases the percentage of Lgr5+ cells to value of about 1% or greater, about 2% or greater, about 3% or greater, about 4% or greater, about 5% or greater, about 10% or greater, about 15% or greater, about 20% or greater, about 25% or greater, about 30% or greater, about 40% or greater, about 50% or greater, about 60% or greater, about 70% or greater, about 80% or greater, about 90% or greater, or about 95% or greater

Embodiment 85. The compound of any one of the preceding embodiments, wherein the compound has a measured Papp (B-A) coefficient of about 0.1 to about 50, about 0.25 to about 40, about 0.5 to about 35, about 0.75 to about 30, about 1 to about 25, about 2 to about 20, about 3 to about 15, about 4 to about 10, about 5 to about 9, about 6 to about 8, or about 6 to about 7.

Embodiment 86. The compound of any one of the preceding embodiments, wherein the compound has a measured Papp (B-A) coefficient of about 0.1 or greater, about 0.25 or greater, about 0.5 or greater, about 0.75 or greater, about 1 or greater, about 2 or greater, about 3 or greater, about 4 or greater, about 5 or greater, about 10 or greater, about 15 or greater, about 20 or greater, about 25 or greater, about 30 or greater, about 35 or greater, about 40 or greater, about 45 or greater, or about 50 or greater.

Embodiment 87. The compound of any one of the preceding embodiments, wherein the compound has a measured efflux ratio of about 0.1 to about 50, about 0.25 to about 40, about 0.5 to about 35, about 0.75 to about 30, about 1 to about 25, about 2 to about 20, about 3 to about 15, about 4 to about 10, about 5 to about 9, about 6 to about 8, or about 6 to about 7.

Embodiment 88. The compound of any one of the preceding embodiments, wherein the compound has a measured efflux ratio of about 50 or less, about 45 or less, about 40 or less, about 35 or less, about 30 or less, about 25 or less, about 20 or less, about 15 or less, about 10 or less, about 5 or less, about 4 or less, about 3 or less, about 2 or less, about 1 or less, about 0.75 or less, about 0.5 or less, about 0.2 or less, or about 0.1 or less.

Embodiment 89. The compound of any one of the preceding embodiments, wherein the compound has a measured concentration of about 0.001 μM to about 100 μM, about 0.002 μM to about 90 μM, about 0.005 μM to about 80 μM, about 0.01 μM to about 70 μM, about 0.05 μM to about 60 μM, about 0.1 μM to about 50 μM, about 0.5 μM to about 40 μM, about 1 μM to about 30 μM, about 2 μM to about 25 μM, about 3 μM to about 20 μM, about 4 μM to about 15 μM, about 5 μM to about 10 μM, about 6 μM to about 9 μM, or about 7 μM to about 8 μM in H₂O at pH 7.4.

Embodiment 90. The compound of any one of the preceding embodiments, wherein the compound has measured concentrations of about 0.001 μM or greater, about 0.002 μM or greater, about 0.005 μM or greater, about 0.01 μM or greater, about 0.05 μM or greater, about 0.1 μM or greater, about 0.5 μM or greater, about 1 μM or greater, about 2 μM or greater, about 3 μM or greater, 4 μM or greater, about 5 μM or greater, about 10 μM or greater, about 25 μM or greater, about 50 μM or greater, or about 100 μM or greater in H₂O at pH 7.4.

Embodiment 91. A pharmaceutical composition comprising a compound of any one of the preceding embodiments, or a pharmaceutically acceptable salt or tautomer thereof, and a pharmaceutically acceptable carrier.

Embodiment 92. The pharmaceutical composition of embodiment 91, further comprising an additional pharmaceutically active agent.

Embodiment 92a. The pharmaceutical composition of embodiment 91, further comprising at least one additional pharmaceutically active agent or a pharmaceutically acceptable salt or tautomer thereof.

Embodiment 92b. The pharmaceutical composition of embodiment 92a, wherein the at least one additional pharmaceutically active agent is valproic acid or a pharmaceutically acceptable salt or tautomer thereof.

Embodiment 92c. The pharmaceutical composition of any one of embodiments 91, and 92a-92b, wherein the at least one additional pharmaceutically active agent is tranylcypromine, or a pharmaceutically acceptable salt or tautomer thereof.

Embodiment 92d. The pharmaceutical composition of any one of embodiments 91 and 92a-92c, wherein the at least one additional pharmaceutically active agent includes tranylcypromine, or a pharmaceutically acceptable salt or tautomer thereof, and valproic acid, or a pharmaceutically acceptable salt or tautomer thereof.

Embodiment 92e. The pharmaceutical composition of any one of embodiments 91 and 92a-92d, wherein the pharmaceutically acceptable salt of valproic acid is sodium valproate.

Embodiment 93. A method of expanding a population of cochlear cells in a cochlear tissue comprising a parent population, the method comprising contacting the cochlear tissue with a compound of any one of the preceding embodiments, or a pharmaceutically acceptable salt or tautomer thereof or a pharmaceutical composition of any one of the preceding embodiments.

Embodiment 94. The method of embodiment 93, wherein the cochlear tissue is in a subject.

Embodiment 95. The method of any one of the preceding embodiments, wherein the contacting the cochlear tissue with the composition is achieved by administering the composition trans-tympanically to the subject.

Embodiment 96. The method of any one of the preceding embodiments, wherein contacting the cochlear tissue with the composition results in improved auditory functioning of the subject.

Embodiment 97. A method of facilitating the generation of tissue cells, the method comprising administering or causing to be administered to a stem cell population a compound of any one of the preceding embodiments, or a pharmaceutically acceptable salt or tautomer thereof or a pharmaceutical composition of any one of the preceding embodiments.

Embodiment 98. The method of embodiment 97, wherein the tissue cells are cochlear cells.

Embodiment 99. The method of claim any one of the preceding embodiments, wherein the tissue cells are inner ear hair cells.

Embodiment 100. A method of treating or preventing a disease associated with absence or lack of certain tissue cells in a subject in need thereof, comprising administering or causing to be administered to a stem cell population a compound of any one of the preceding embodiments, or a pharmaceutically acceptable salt or tautomer thereof or a pharmaceutical composition of any one of the preceding embodiments.

Embodiment 101. The method of embodiment 100, wherein the tissue cells are cochlear cells.

Embodiment 102. The method of any one of the preceding embodiments, wherein the tissue cells are inner ear hair cells.

Embodiment 103. A method of treating or preventing hearing loss in a subject in need thereof, the method comprising administering a compound of any one of the preceding embodiments, or a pharmaceutically acceptable salt or tautomer thereof or a pharmaceutical composition of any one of the preceding embodiments.

Embodiment 103a. The method of embodiment 103, wherein the hearing loss is sensorineural hearing loss.

Embodiment 104. The method of embodiment 103, wherein the compound is administered trans-tympanically to the subject (e.g., to a cochlear tissue of the subject).

Embodiment 105. A method of facilitating the generation of inner ear hair cells, the method comprising: administering a compound of any one of the preceding embodiments or a pharmaceutically acceptable salt thereof, alone or in combination with an additional pharmaceutically active agent, to expand the stem cell population of cochlear tissue.

Embodiment 106. A method of regenerating or improving hearing in a mammal, the method comprising administering a compound of any one of the preceding embodiments, or a pharmaceutically acceptable salt or tautomer thereof, alone or in combination with an additional pharmaceutically active agent.

Embodiment 107 The method of embodiment 105 or embodiment 106, wherein the administration is to a stem cell population is of an in vivo subject.

Embodiment 108. A method of generating inner ear hair cells, the method comprising administering a compound of any one of the preceding embodiments, or a pharmaceutically acceptable salt or tautomer thereof, alone or in combination with an additional pharmaceutically active agent, wherein the method proliferates Lgr5⁺ cells in an initial population in vivo, resulting in an expanded population of Lgr5⁺ cells, resulting in generation of inner ear hair cells.

Embodiment 109. A method of facilitating generation of intestinal cells, the method comprising administering a compound of any one of the preceding embodiments or a pharmaceutically acceptable salt thereof, alone or in combination with an additional pharmaceutically active agent, to expand the stem cell population of intestinal epithelia.

Embodiment 110. The method of embodiment 109, wherein the intestinal epithelia is regenerated.

Embodiment 111. The method of any one of the preceding embodiments, wherein the method is a treatment for promoting repair of damaged mucosa related to chemotherapy-induced gastrointestinal mucositis, Graph Versus Host Disease, gastric ulcer, Crohns, or ulcerative colitis.

Embodiment 112. A method of expanding Lgr5⁺ cell population of intestinal epithelia, the method comprising: administering a compound of any one of the preceding embodiments or a pharmaceutically acceptable salt thereof, alone or in combination with an additional pharmaceutical agent.

Embodiment 113. A method of use of a compound of any one of the preceding embodiments, or a pharmaceutically acceptable salt or tautomer thereof, alone or in combination with an additional pharmaceutically active agent to regenerate Lgr5⁺ cell population intestinal cells in a mammal.

Embodiment 114. The method of embodiment 113 wherein the method is a treatment for promoting the repair of damaged mucosa related to chemotherapy-induced gastrointestinal mucositis, Graph Versus Host Disease, gastric ulcer, Crohns, or ulcerative colitis.

Embodiment 115. A method of proliferating Lgr5⁺ epithelial cells in in vivo, the method comprising: administering a compound of any one of the preceding embodiments or a pharmaceutically acceptable salt thereof.

Embodiment 116. A method for expanding a population of vestibular cells in a vestibular tissue comprising contacting the vestibular tissue with (i) a compound of any one of the preceding embodiments or a pharmaceutically acceptable salt thereof, and (ii) an additional pharmaceutically active agent to form an expanded population of cells in the vestibular tissue.

Embodiment 117. A method of treating or preventing vestibular diseases, alopecia, oncology, acute myeloid leukemia, inflammation, Alzheimer's disease, Huntington's disease, Friedreick's ataxia, depression, anxiety, manic episodes of bipolar/mood disorders, Parkinson's, diabetes, bacterial infection, Anti-Trypanosoma brucei, ischemia, heart disease, vascular degeneration, and/or platelet aggregation in a subject in need thereof, the method comprising administering a compound of any one of the preceding embodiments or a pharmaceutically acceptable salt thereof, alone or in combination with an additional pharmaceutically active agent.

Embodiment 118. The method of any one of the preceding embodiments, wherein the compound of any one of the preceding embodiments functions as an inhibitor of at least one of FOXO-1, GSK3 α/β and LSD-1.

Embodiment 118a. The method of any one of the preceding embodiments, wherein the compound of any one of the preceding embodiments functions as an inhibitor of at least two of FOXO-1, GSK3 α/β and LSD-1.

Embodiment 118b. The method of any one of the preceding embodiments, wherein the compound of any one of the preceding embodiments functions as an inhibitor of FOXO-1, GSK3 α/β and LSD-1.

Embodiment 119. A method of inhibiting LSD, GSK3, and/or FOXO in a cell, the method comprising contacting the cell with a compound of any one of the preceding embodiments or a pharmaceutically acceptable salt thereof.

Embodiment 120. The method of any one of the preceding embodiments, wherein the compound of any one of the preceding embodiments functions as a cell cycle progression pathway agonist.

Embodiment 121. The method of any one of the preceding embodiments, wherein the additional pharmaceutical agent is an HDAC inhibitor and/or a poloxamer.

Embodiment 122. A system for treating or preventing a disease associated with absence or lack of certain tissue cells in a subject in need thereof, comprising administering: a compound of any one of the preceding embodiments, or a pharmaceutically acceptable salt or tautomer thereof; and a trans-tympanic administrative device.

Embodiment 123. A compound of any one of the preceding embodiments, or a pharmaceutically acceptable salt or tautomer thereof, for use in treating or preventing a disease associated with absence or lack of certain tissue cells in a subject in need thereof.

Embodiment 124. A compound of any one of the preceding embodiments, or a pharmaceutically acceptable salt or tautomer thereof, for use in treating or preventing hearing loss in a subject in need thereof.

Embodiment 124a. The compound, or a pharmaceutically acceptable salt or tautomer thereof, for use according to embodiment 104, wherein the hearing loss is sensorineural hearing loss.

Embodiment 125. Use of a compound of any one of the preceding embodiments, or a pharmaceutically acceptable salt or tautomer thereof, in the manufacture of a medicament for treating or preventing a disease associated with absence or lack of certain tissue cells in a subject in need thereof.

Embodiment 126. Use of a compound of any one of the preceding embodiments, or a pharmaceutically acceptable salt or tautomer thereof, in the manufacture of a medicament for treating or preventing hearing loss in a subject in need thereof.

Embodiment 126a. The use according to embodiment 126, wherein the hearing loss is sensorineural hearing loss.

Embodiment 127. Use of a compound of any one of the preceding embodiments, or a pharmaceutically acceptable salt or tautomer thereof, in the manufacture of a medicament for treating or preventing a disease responding to LSD inhibition, GSK3 inhibition, and/or FOXO inhibition in a subject in need thereof.

Embodiment 128. Use of a compound of any one of the preceding embodiments, or a pharmaceutically acceptable salt or tautomer thereof, in the manufacture of a medicament for treating or preventing vestibular diseases, alopecia, oncology, acute myeloid leukemia, inflammation, Alzheimer's disease, Huntington's disease, Friedreick's ataxia, depression, anxiety, manic episodes of bipolar/mood disorders, Parkinson's, diabetes, bacterial infection, Anti-Trypanosoma brucei, ischemia, heart disease, vascular degeneration, and/or platelet aggregation in a subject in need thereof.

Embodiment 129. The compound, the method, or the use of any one of the preceding embodiments, wherein the administration of the compound to a subject results into a higher Lgr5+ cell number in the subject, as compared to a comparable subject not being administered with the compound, by a factor ranging from about 2 fold to about 2,000,000 fold, from about 10 fold to about 1,000,000 fold, from about 100 fold to about 100,000 fold, or from about 1,000 fold to about 10,000 fold.

Embodiment 130. The compound, the method, or the use of any one of the preceding embodiments, wherein the administration of the compound to a subject results into a higher Lgr5+ cell number in the subject, as compared to a comparable subject not being administered with the compound, by a factor of greater than about 10 fold, greater than about 10,000 fold, greater than about 100,000 fold, or greater than about 1,000,000 fold.

Embodiment 131. The compound, the method, or the use of any one of the preceding embodiments, wherein the administration of the compound to a subject results into a higher Lgr5+ cell number in the subject, as compared to a comparable subject being administered with a Wnt agonist, by a factor ranging from about 0.1 fold to about 10 fold, from about 0.5 fold to about 5 fold, from about 1 fold to about 4 fold, or from about 1.5 fold to about 3 fold.

Embodiment 132. The compound, the method, or the use of any one of the preceding embodiments, wherein the administration of the compound to a subject results into a higher Lgr5+ cell number in the subject, as compared to a comparable subject being administered with a Wnt agonist, by a factor of greater than about 1.5 fold, greater than about 2 fold, greater than about 2.5 fold, greater than about 3 fold, greater than about 3.5 fold, or greater than about 4 fold.

Embodiment 133. The compound, the method, or the use of any one of the preceding embodiments, wherein the administration of the compound in combination with sodium valproate to a subject results into a higher percentage of Lgr5+ cells in the subject, as compared to a comparable subject being administered with the compound without sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

Embodiment 134. The compound, the method, or the use of any one of the preceding embodiments, wherein the administration of the compound in combination with sodium valproate to a subject results into a higher percentage of Lgr5+ cells in the subject, as compared to a comparable subject being administered with the compound without sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

Embodiment 135. The compound, the method, or the use of any one of the preceding embodiments, wherein the administration of the compound in combination with an LSD-1 inhibitor to a subject results into a higher percentage of Lgr5+ cells in the subject, as compared to a comparable subject being administered with the compound without the LSD-1 inhibitor, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

Embodiment 136. The compound, the method, or the use of any one of the preceding embodiments, wherein the administration of the compound in combination with an LSD-1 inhibitor to a subject results into a higher percentage of Lgr5+ cells in the subject, as compared to a comparable subject being administered with the compound without the LSD-1 inhibitor, by a factor of greater than about 1 fold, greater than about 1.5 fold, greater than about 2 fold, greater than about 2.5 fold, greater than about 3 fold, greater than about 3.5 fold, or greater than about 4 fold.

Embodiment 137. The compound, the method, or the use of any one of the preceding embodiments, wherein the administration of the compound in combination with sodium valproate and an LSD-1 inhibitor to a subject increases the percentage of Lgr5+ cells in the subject, as compared to a comparable subject being administered with the compound and sodium valproate without the LSD-1 inhibitor, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

Embodiment 138. The compound, the method, or the use of any one of the preceding embodiments, wherein the administration of the compound in combination with sodium valproate and an LSD-1 inhibitor to a subject increases the percentage of Lgr5+ cells in the subject, as compared to a comparable subject being administered with the compound and sodium valproate without the LSD-1 inhibitor, by a factor of greater than about 1 fold, greater than about 1.5 fold, greater than about 2 fold, greater than about 2.5 fold, greater than about 3 fold, greater than about 3.5 fold, or greater than about 4 fold.

Embodiment 139. The compound, the method, or the use of any one of the preceding embodiments, wherein the administration of the compound in combination with sodium valproate and an LSD-1 inhibitor to a subject increases the percentage of Lgr5+ cells in the subject, as compared to a comparable subject being administered with the compound and the LSD-1 inhibitor without sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

Embodiment 140. The compound, the method, or the use of any one of the preceding embodiments, wherein the administration of the compound in combination with sodium valproate and an LSD-1 inhibitor to a subject increases the percentage of Lgr5+ cells in the subject, as compared to a comparable subject being administered with the compound and the LSD-1 inhibitor without sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

Embodiment 141. The compound, the method, or the use of any one of the preceding embodiments, wherein the administration of the compound in combination with sodium valproate and an LSD-1 inhibitor to a subject increases the percentage of Lgr5+ cells in the subject, as compared to a comparable subject being administered with the compound without the LSD-1 inhibitor or sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

Embodiment 142. The compound, the method, or the use of any one of the preceding embodiments, wherein the administration of the compound in combination with sodium valproate and an LSD-1 inhibitor to a subject increases the percentage of Lgr5+ cells in the subject, as compared to a comparable subject being administered with the compound without the LSD-1 inhibitor or sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

Embodiment 143. The compound, the method, or the use of any one of the preceding embodiments, wherein when a cell population is contacted with the compound, the contacting results into a higher Lgr5+ cell number in the cell population, as compared to a comparable cell population not being contacted with the compound, by a factor ranging from about 2 fold to about 2,000,000 fold, from about 10 fold to about 1,000,000 fold, from about 100 fold to about 100,000 fold, or from about 1,000 fold to about 10,000 fold.

Embodiment 144. The compound, the method, or the use of any one of the preceding embodiments, wherein when a cell population is contacted with the compound, the contacting results into a higher Lgr5+ cell number in the cell population, as compared to a comparable cell population not being contacted with the compound, by a factor of greater than about 10 fold, greater than about 10,000 fold, greater than about 100,000 fold, or greater than about 1,000,000 fold.

Embodiment 145. The compound, the method, or the use of any one of the preceding embodiments, wherein when a cell population is contacted with the compound, the contacting results into a higher Lgr5+ cell number in the cell population, as compared to a comparable cell population being contacted with a Wnt agonist, by a factor ranging from about 0.1 fold to about 10 fold, from about 0.5 fold to about 5 fold, from about 1 fold to about 4 fold, or from about 1.5 fold to about 3 fold.

Embodiment 146. The compound, the method, or the use of any one of the preceding embodiments, wherein when a cell population is contacted with the compound, the contacting results into a higher Lgr5+ cell number in the cell population, as compared to a comparable cell population being contacted with a Wnt agonist, by a factor of greater than about 1.5 fold, greater than about 2 fold, greater than about 2.5 fold, greater than about 3 fold, greater than about 3.5 fold, or greater than about 4 fold.

Embodiment 147. The compound, the method, or the use of any one of the preceding embodiments, wherein when a cell population is contacted with the compound in combination with sodium valproate, the contacting results into a higher percentage of Lgr5+ cells in the cell population, as compared to a comparable cell population being contacted with the compound without sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

Embodiment 148. The compound, the method, or the use of any one of the preceding embodiments, wherein when a cell population is contacted with the compound in combination with sodium valproate, the contacting results into a higher percentage of Lgr5+ cells in the cell population, as compared to a comparable cell population being contacted with the compound without sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

Embodiment 149. The compound, the method, or the use of any one of the preceding embodiments, wherein when a cell population is contacted with the compound in combination with an LSD-1 inhibitor, the contacting results into a higher percentage of Lgr5+ cells in the cell population, as compared to a comparable cell population being contacted with the compound without the LSD-1 inhibitor, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

Embodiment 150. The compound, the method, or the use of any one of the preceding embodiments, wherein when a cell population is contacted with the compound in combination with an LSD-1 inhibitor, the contacting results into a higher percentage of Lgr5+ cells in the cell population, as compared to a comparable cell population being contacted with the compound without the LSD-1 inhibitor, by a factor of greater than about 1 fold, greater than about 1.5 fold, greater than about 2 fold, greater than about 2.5 fold, greater than about 3 fold, greater than about 3.5 fold, or greater than about 4 fold.

Embodiment 151. The compound, the method, or the use of any one of the preceding embodiments, wherein when a cell population is contacted with the compound in combination with sodium valproate and an LSD-1 inhibitor, the contacting increases the percentage of Lgr5+ cells in the cell population, as compared to a comparable cell population being contacted with the compound and sodium valproate without the LSD-1 inhibitor, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

Embodiment 152. The compound, the method, or the use of any one of the preceding embodiments, wherein when a cell population is contacted with the compound in combination with sodium valproate and an LSD-1 inhibitor, the contacting increases the percentage of Lgr5+ cells in the cell population, as compared to a comparable cell population being contacted with the compound and sodium valproate without the LSD-1 inhibitor, by a factor of greater than about 1 fold, greater than about 1.5 fold, greater than about 2 fold, greater than about 2.5 fold, greater than about 3 fold, greater than about 3.5 fold, or greater than about 4 fold.

Embodiment 153. The compound, the method, or the use of any one of the preceding embodiments, wherein when a cell population is contacted with the compound in combination with sodium valproate and an LSD-1 inhibitor, the contacting increases the percentage of Lgr5+ cells in the cell population, as compared to a comparable cell population being contacted with the compound and the LSD-1 inhibitor without sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

Embodiment 154. The compound, the method, or the use of any one of the preceding embodiments, wherein when a cell population is contacted with the compound in combination with sodium valproate and an LSD-1 inhibitor, the contacting increases the percentage of Lgr5+ cells in the cell population, as compared to a comparable cell population being contacted with the compound and the LSD-1 inhibitor without sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

Embodiment 155. The compound, the method, or the use of any one of the preceding embodiments, wherein when a cell population is contacted with the compound in combination with sodium valproate and an LSD-1 inhibitor, the contacting increases the percentage of Lgr5+ cells in the cell population, as compared to a comparable cell population being contacted with the compound without the LSD-1 inhibitor or sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

Embodiment 156. The compound, the method, or the use of any one of the preceding embodiments, wherein when a cell population is contacted with the compound in combination with sodium valproate and an LSD-1 inhibitor, the contacting increases the percentage of Lgr5+ cells in the cell population, as compared to a comparable cell population being contacted with the compound without the LSD-1 inhibitor or sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

### EXAMPLES

### Abbreviations

AcOH acetic acid

aq aqueous

atm atmosphere

bm broad multiplet

brs broad singlet

CDCl₃ chloroform-d

d doublet

DCE dichloroethane

DCM dichloromethane

DMF N,N-dimethylformamide

DMSO dimethylsulfoxide

DMSO-d₆ hexadeuterodimethylsulfoxide

DPPA diphenylphosphoryl azide

ESI electrospray ionization

EtI ethyl iodide

EtOAc ethyl acetate

EtOH ethanol

g gram

hrs hour(s)

¹H NMR proton nuclear magnetic resonance spectroscopy

HPLC high performance liquid chromatography

LC-MS liquid chromatography-mass spectrometry

LDA lithium diisopropylamide

m multiplet

MeOH methanol

mg milligram

MHz megahertz

min minute(s)

mL milliliter

mmol millimole

MS mass spectrometry

MTBE methyl tert-butyl ether

nBuLi n-butyllithium

NMR nuclear magnetic resonance

Pd/C palladium on carbon

PE petroleum ether

PPE polyphosphate ester

ppm parts per million

psi pounds per square inch

q quartet

RT room temperature

s singlet

triplet

TBAF tetrabutylammonium fluoride

tBuOH tert butyl alcohol

tBuOK potassium tert butoxide

TEA triethylamine

TFA trifluoroacetic acid

THE tetrahydrofuran

TLC thin layer chromatography

TMSCI trimethylsilyl chloride

General Synthetic Information

General Synthetic Scheme 1: Synthesis of 6-fluoro-7-amino quinolones

The starting Intermediate 1 can be purchased or prepared. Some synthetic references are: “Developing ciprofloxacin analogues against plant DNA gyrase: a novel herbicide mode of action” Chemical Communications, 2018, 54(15), 1869-1872; “Operative conversions of 3-carboxy-4-quinolones into 3-nitro-4-quinolones via ipso-nitration: potential antifilarial agents as inhibitors of Brugia malayi thymidylate kinase” RSC Advances 2015, 5(100), 82208-82214; “An operational transformation of 3-carboxy-4-quinolones into 3-nitro-4-quinolones via ipso-nitration using polysaccharide supported copper nanoparticles: synthesis of 3-tetrazolyl bioisosteres of 3-carboxy-4-quinolones as antibacterial agents” RSC Advances 2016, 6(23), 19052-19059; “Mechanism and synthesis of pharmacologically active quinolones from Morita-Baylis-Hillman adducts” Tetrahedron 2010, 66(24), 4370-4376; “Synthesis and antibacterial activity of novel fluoroquinolone analogs” Medicinal Chemistry Research 2014, 23(12), 5237-524; and “Enhancement of the Antimalarial Activity of Ciprofloxacin Using a Double Prodrug/Bioorganometallic Approach” Journal of Medicinal Chemistry (2009), 52(24), 7954-7957, which are all incorporated by reference.

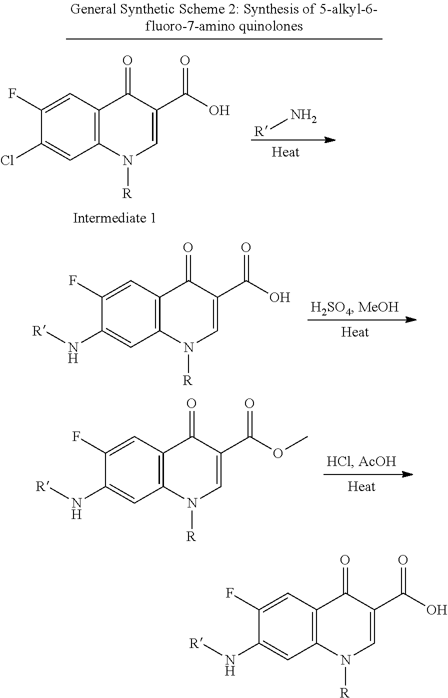

A synthetic reference is: “Synthesis and Biological Properties of Substituted 1,4-Dihydro-5-methyl-4-oxo-3-quinolinecarboxylic Acids” Bioorganic Medicinal Chemistry (1995), 3(12), 1699-706, which is incorporated by reference.

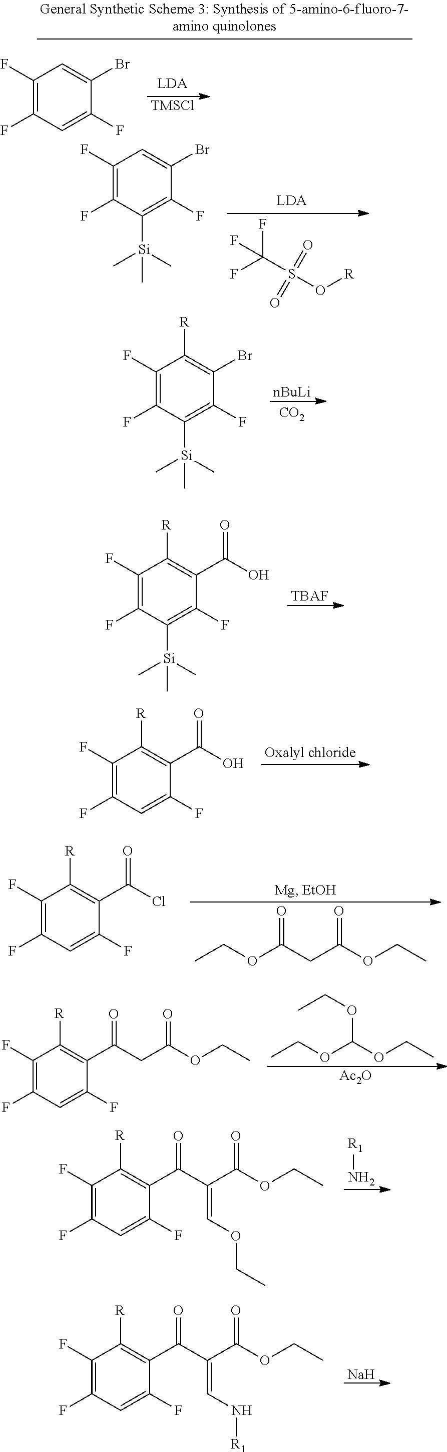

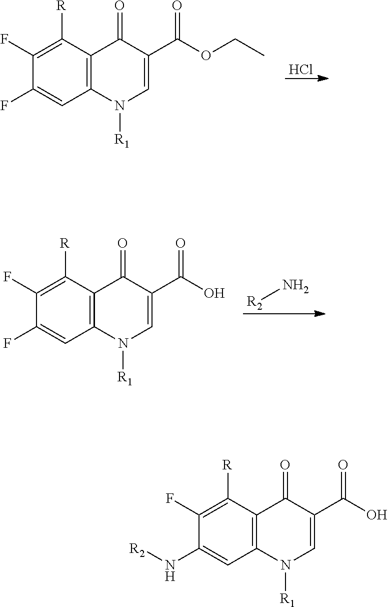

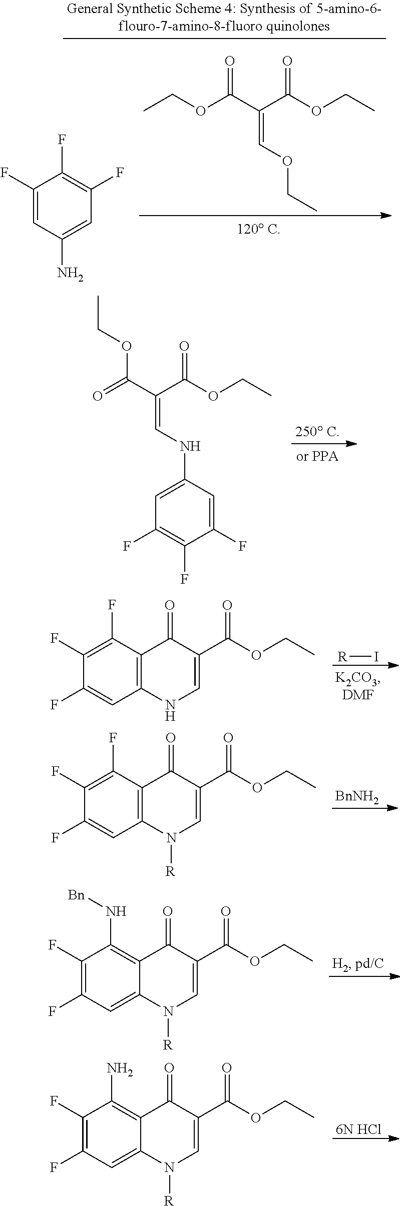

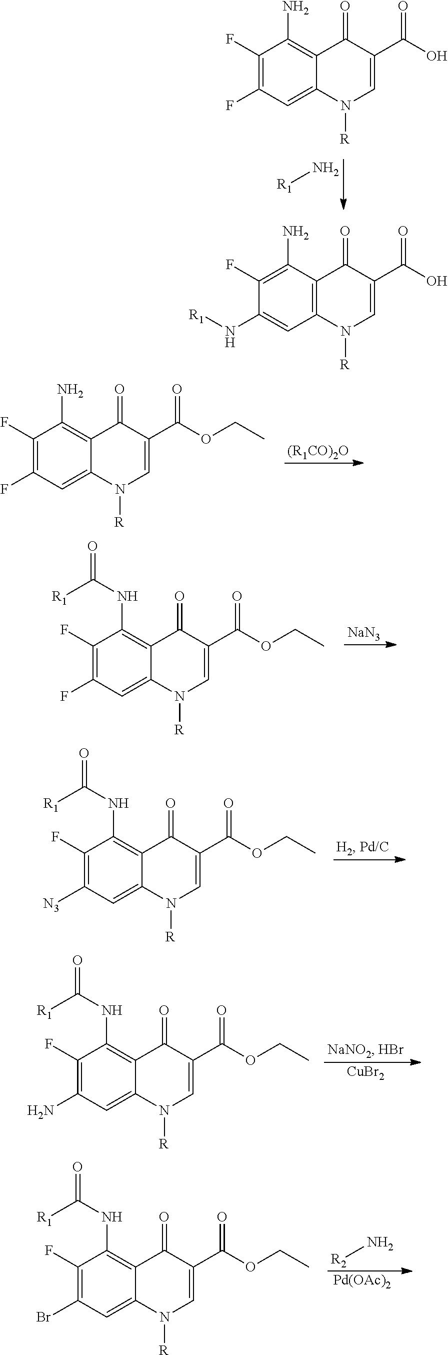

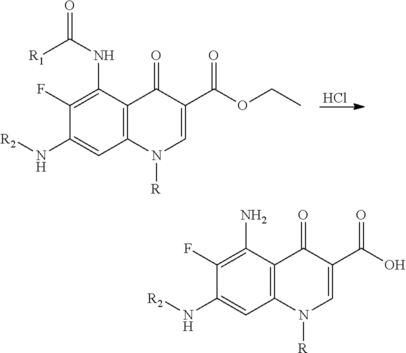

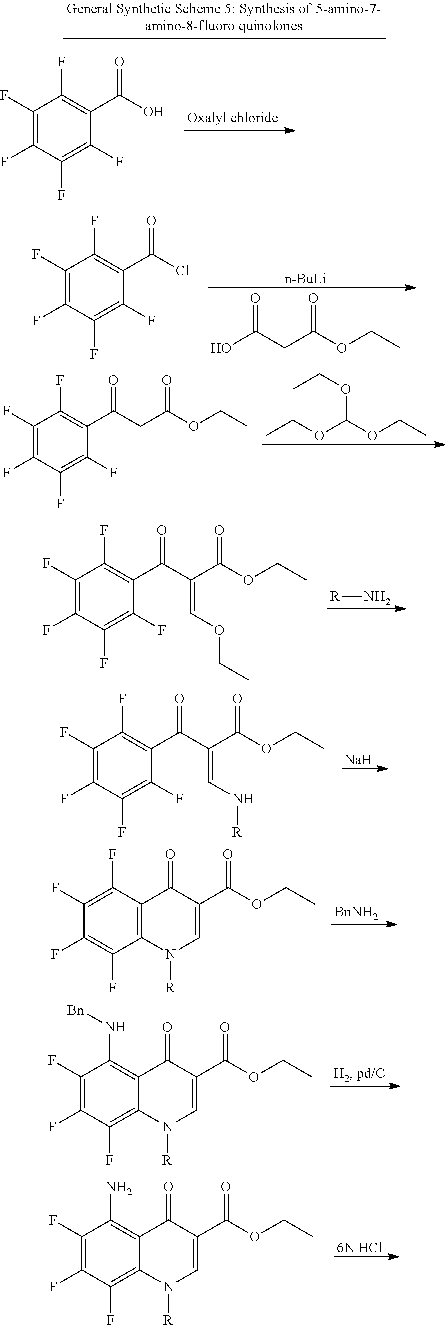

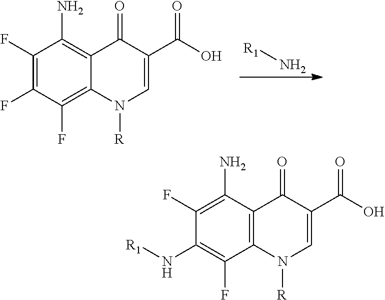

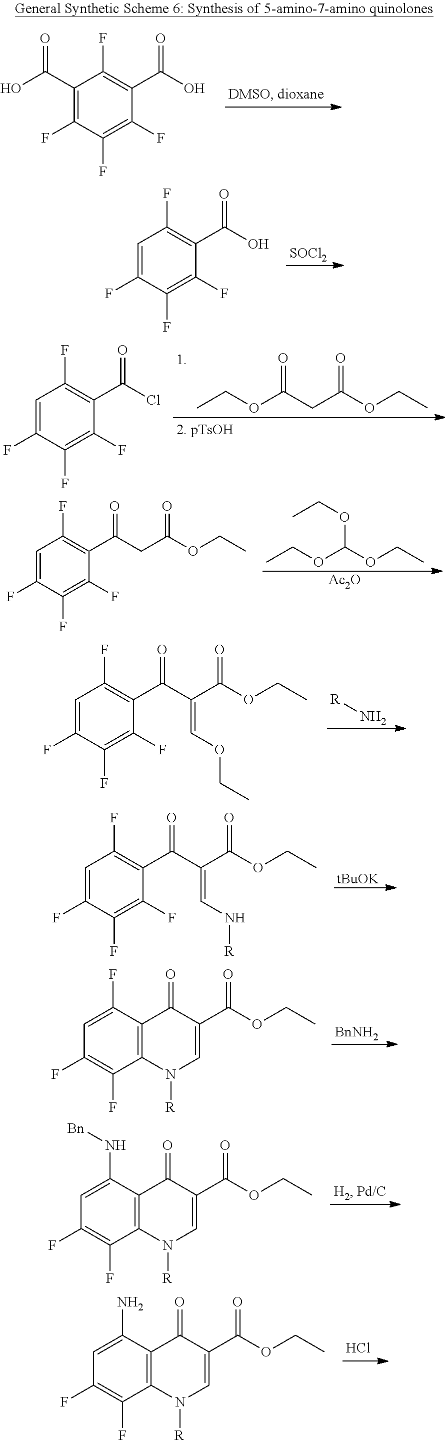

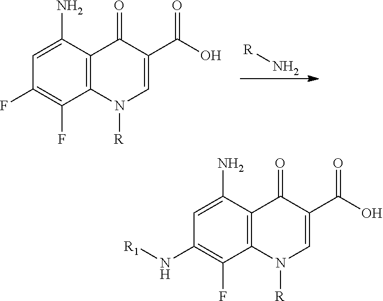

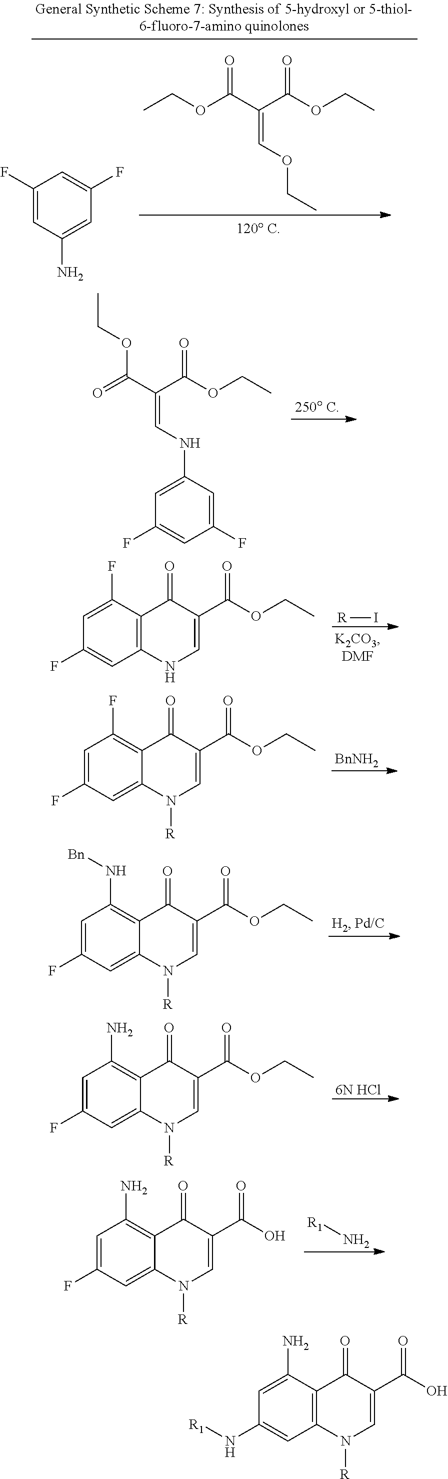

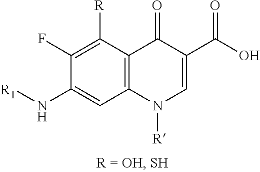

Some synthetic references are: “Synthesis and biological activity of 5-amino- and 5-hydroxyquinolones, and the overwhelming influence of the remote N1-substituent in determining the structure-activity relationship” J. Med. Chem. 1991, 34, 1142-52; and “Synthesis and Structure-Activity Relationships of 5-Substituted-6,8-Difluoroquinolones, Including Sparfloxacin, a New Quinolone Antibacterial Agent with Improved Potency” J. Med. Chem. 1990, 33, 1645-1656; which are all incorporated by reference.

General Side Chain Synthetic Schemes

General Synthetic Scheme 8: Synthesis of Cyclopropyl Amine Side Chains Using Reductive Amination

A synthetic reference is: European Journal of Medicinal Chemistry 92 (2015) 377-386, which is incorporated by reference.

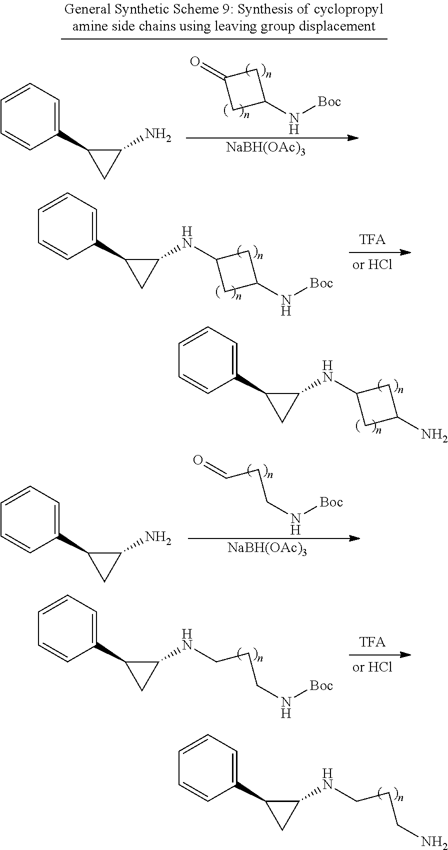

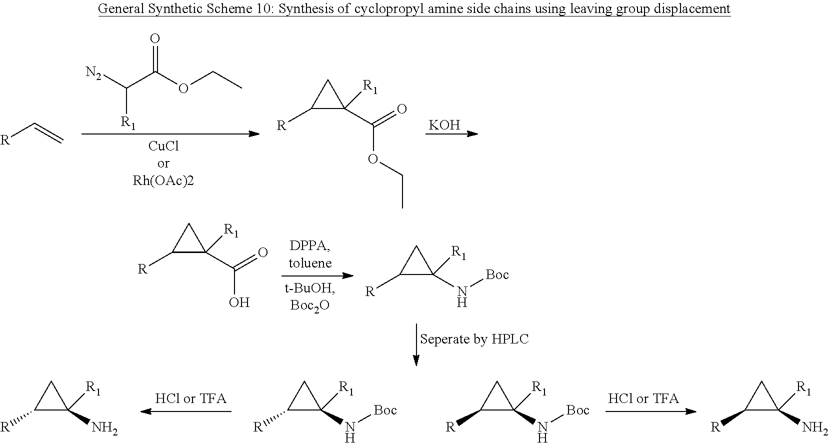

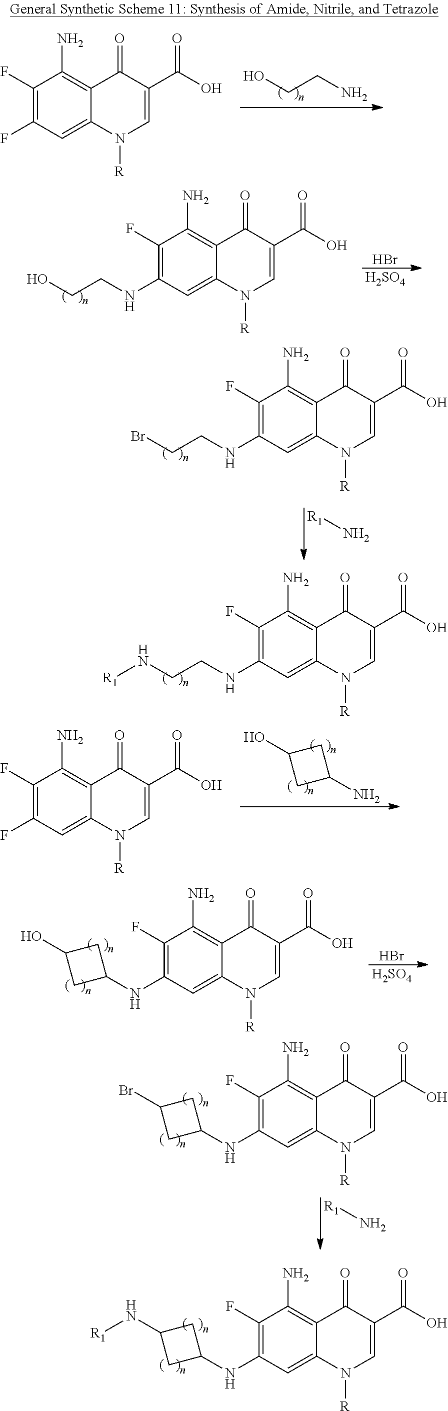

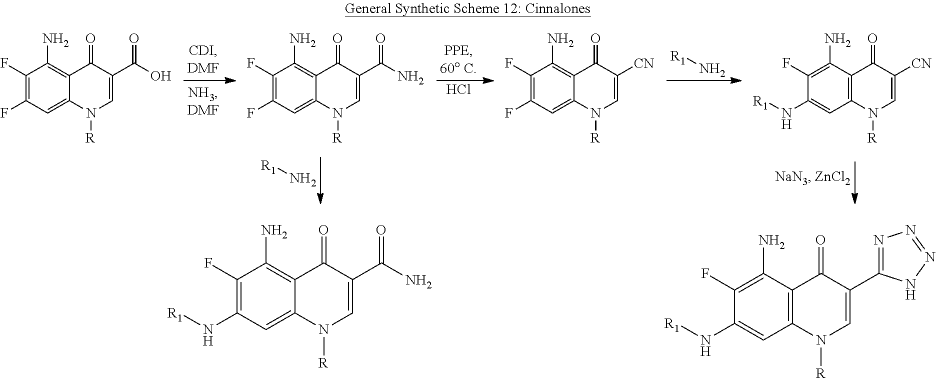

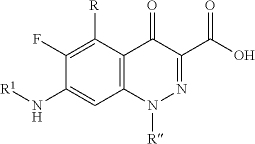

Some synthetic references are: “A new cinnalone ring construction by the reaction 2-Diazo-3-2-(fluorophenyl)-3-oxoproprionates Tri-n-butylphosphine” Chemical Pharmaceutical Bulletin (1988), 36(4), 1321-1327; “Fluorocinnoline derivatives. II. Synthesis and antibacterial activity of fluorinated 1-alkyl-1,4-dihydro-4-oxocinnoline-3-carboxylic acids” Chemical Pharmaceutical Bulletin (1989), 37(1), 93-99; “Cinoxacin analogs as potential antibacterial agents: synthesis and antibacterial activity” Quinolones (1989), 109-17; “Synthesis and antibacterial activity of some 1-aryl-1,4-dihydro-4-oxocinnoline-3-carboxylic acids” Collection of Czechoslovak Chemical Communications (1990), 55(5), 1311-20; and “Unexpected reactivity of trifluoromethyl diazomethane (CF₃CHN₂): Electrophilicity of the terminal N-atom” Organic Letters (2016), 18(14), 3406-3409; and “Synthesis of Fluorine-Containing 2-(1-Aryl-4-oxo-1,4-dihydrocinnolin-3-yl)-2-oxoacetic Acids” Russian Journal of Organic Chemistry (2005), 41(9), 1374-1376; which are all incorporated by reference.

### Example 1: Synthesis of Compound I-1 and Compound I-2

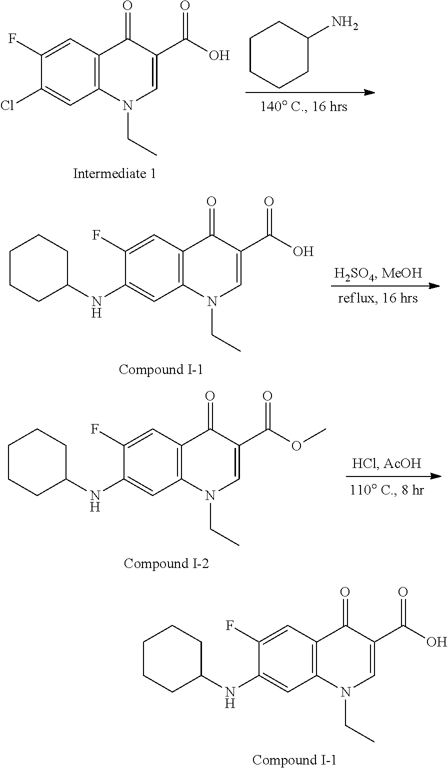

### Synthesis of Compound I-1

To a solution of Intermediate 1 (0.5 g, 1.9 mmol) in N-Methyl-2-pyrrolidone (1 mL) was added cyclohexylamine (4 mL), then the mixture was stirred at 140° C. for 16 hours. The mixture was concentrated in vacuum to give a residue, which was partially purified by silica gel chromatography to give Compound I-1 (5.0 g) as a brown solid.

### Synthesis of Compound I-2

To a solution of partially purified Compound I-1 (5 g, crude) in MeOH (75 mL) was added H₂SO₄ (5 mL, 18 mol/L). The mixture was heated to reflux for 16 hours, the mixture was poured into ice-water, adjusted pH=7-8 and extracted with dichloromethane, the combined organic phases was concentrated in vacuum to give crude product, which was purified by silica gel chromatography to give Compound I-2 (0.3 g) as a white solid.

### Synthesis of Compound I-1

To a mixture of Compound I-2 (0.3 g, crude) in H₂O (3 mL) was added HCl (12 N, 3 mL) and AcOH (3 mL). The mixture was stirred at 110° C. for 8 hours, the mixture was poured into ice-water, filtered to give product, which was recrystallized from dimethyl sulfoxide (DMSO) to give Compound I-1 (66 mg, 22.9%) as a white solid. ¹H NMR (DMSO-d₆, 400 MHz): δ (ppm) 15.79 (s, 1H), 8.84 (s, 1H), 7.78-7.81 (d, 1H, J=12.4 Hz), 6.78-6.80 (d, 1H, J=7.2 Hz), 6.66-6.68 (d, 1H, J=2.0 Hz), 4.51-4.56 (q, 2H), 3.61-3.63 (m, 1H), 1.94-1.97 (d, 2H, J=5.6 Hz), 1.74-1.78 (m, 2H), 1.64-1.67 (d, 1H, J=13.2 Hz), 1.31-1.41 (m, 7H), 1.15-1.19 (m, 1H); MS: 332.2 [M+1].

### Example 2: Synthesis of Compound I-3

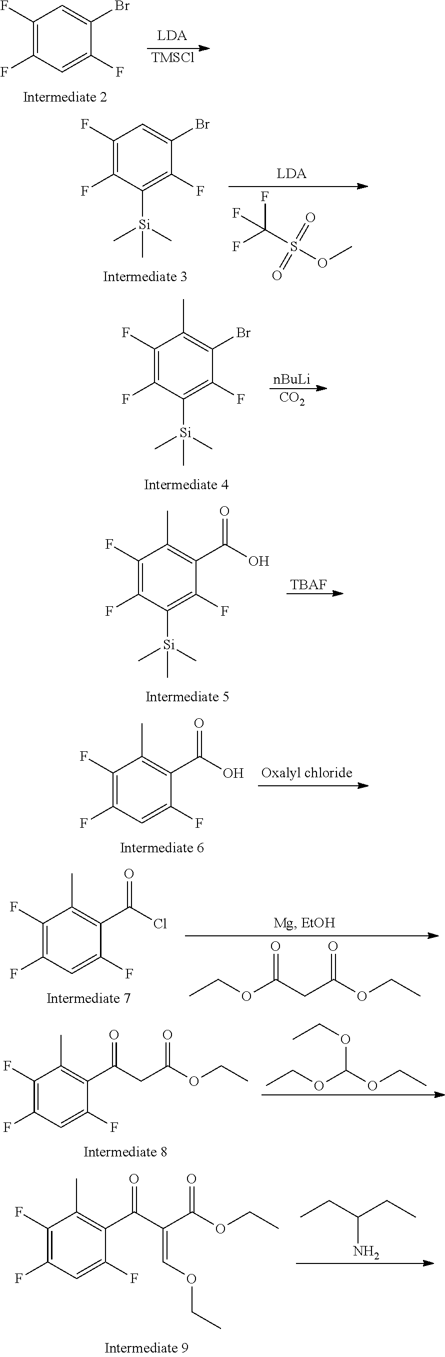

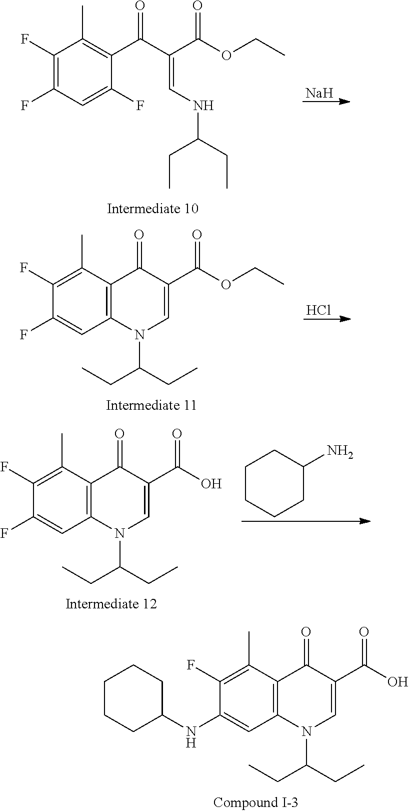

### Synthesis of Intermediate 3

To a solution of Intermediate 2 (94 g, 0.45 mol) in THE (940 mL) was added lithium diisopropylamide (LDA) (295 mL, 0.59 mol) at −78° C. The mixture was stirred at −78° C. for 1.5 hours. Chlorotrimethylsilane (130 g, 1.2 mol) was added dropwise, the mixture was stirred at −78° C. for 30 minutes, then room temperature (RT) for 30 min, the mixture was poured into ice-water, extracted with EtOAc, and concentrated. The crude product was distilled at 70-80° C. to give Intermediate 3 (80 g, 63%) as colorless oil. ¹H NMR (CDCl₃, 400 MHz): δ (ppm) 7.34-7.40 (m, 1H), 0.37 (s, 9H).

### Synthesis of Intermediate 4

To a solution of Intermediate 3 (40 g, 0.14 mol) in THF (400 mL) was added LDA (85 mL, 0.17 mol) at −78° C. under N2, the mixture was stirred at −78° C. for 1.5 hours. Compound methyl trifluoromethanesulfonate (37.7 g, 0.23 mol) was added dropwise, the mixture was stirred at −78° C. for 15 min then warm up to 0° C. for 20 min, the mixture was poured into NaHCO₃ (aq), extracted with EtOAc, the organic phase was washed with brine, dried over Na₂SO₄ and concentrated in vacuum. The crude material was purified by silica gel chromatography to give Intermediate 4 (30 g, 71.4%) as colorless oil. ¹H NMR (CDCl₃, 400 MHz): δ (ppm) 2.37-2.38 (d, 3H, J=2.8 Hz), 0.37 (s, 9H).

### Synthesis of Intermediate 5

To a solution of Intermediate 4 (20 g, 67.3 mmol) in THF (200 mL) was added n-BuLi (28 mL, 70.7 mmol) at −78° C. under nitrogen, the mixture was stirred at −78° C. for 1.5 hours. The mixture was poured into CO₂ (solid) in THF (50 mL), the mixture was stirred at RT for 1 hour, concentrated, Na₂CO₃ (aq) was added, extracted with petroleum ether. The aqueous phase was adjusted pH=3-4, extracted with dichloromethane, the organic phase was washed with brine, dried over Na₂SO₄ and concentrated in vacuum to give Intermediate 5 (10 g, 56.7%) as off-white solid. ¹H NMR (CDCl₃, 400 MHz): δ (ppm) 2.41-2.42 (d, 3H, J=2.8 Hz), 0.39 (s, 9H).

### Synthesis of Intermediate 6

To a solution of Intermediate 5 (10 g, 38.2 mmol) in tetrahydrofuran (THF) (100 mL) was added Tetrabutylammolnium fluoride (TBAF) (36 g, 114.5 mmol), the mixture was heated to reflux for 16 hours. The mixture was concentrated, HCl (2N) was added, extracted with methyl t-butyl ether (MTBE), the organic phase was washed with HCl (2N), brine, dried over Na₂SO₄ and concentrated in vacuum to give a crude, heptane was added, the mixture was stirred at 0-10° C. for 30 min, filtered to give Intermediate 6 (5 g, 69%) as off-white solid. ¹H NMR (CDCl₃, 400 MHz): δ (ppm) 9.77 (s, 1H), 6.85-6.92 (m, 1H), 2.46-2.47 (d, 3H, J=2.4 Hz).

### Synthesis of Intermediate 7

To a solution of Intermediate 6 (5.7 g, 30 mmol) in dichloromethane (60 mL) was added oxalyl chloride (4.6 g, 36 mmol) at 0-10° C., dimethylformamide (DMF) (18 drops, cat) was added dropwise, the mixture was stirred at 30° C. for 3 hours. The mixture was concentrated in vacuum to give Intermediate 7 (6 g, crude) as a yellow oil.

### Synthesis of Intermediate 8

A mixture of Mg (0.86 g, 36 mmol) in EtOH (30 mL) and CCl4 (0.6 mL) was heated at 80° C. for 30 min, diethyl malonate (5.8 g, 36 mmol) was added, the mixture solution was heated to reflux for 2 hours, cooled, Intermediate 7 (6.0 g, crude) in THE (15 mL) was added at 0-5° C., the mixture was stirred at 30° C. for 1 hour. The mixture was poured into HCl (2 N), extracted with EtOAc, the organic phase was washed with brine, dried over Na₂SO₄ and concentrated in vacuum. The crude material was purified by silica gel chromatography to give Intermediate 8 (3.5 g, 44.9%) as a yellow oil.

### Synthesis of Intermediate 9

A mixture of Intermediate 8 (7 g, 27 mmol), triethoxymethane (79 g, 540 mmol) in acetic anhydride (70 mL) was heated to 140° C. for 5 hours, the mixture was concentrated, toluene was added, concentrated in vacuum to give a crude of Intermediate 9 (8 g, crude) as brown oil.

### Synthesis of Intermediate 10

To a solution of Intermediate 9 (8.0 g, crude) in EtOH (60 mL) was added pentan-3-amine (8 mL) at 0-10° C., then the mixture was stirred at 28° C. for 2 hours. The mixture was concentrated in vacuum to give a residue, which was purified by silica gel chromatography to give Intermediate 10 (5.6 g, 62.2%) as a yellow solid. ¹H NMR (CDCl₃, 400 MHz): δ (ppm) 11.09 (s, 1H), 8.14-8.18 (d, 1H, J=14.4 Hz), 6.71-6.77 (m, 1H), 3.92-4.02 (m, 2H), 3.12-3.20 (m, 1H), 2.17 (s, 3H), 1.02-1.78 (m, 4H), 0.87-1.02 (m, 9H); MS: 358.2 [M+1].

### Synthesis of Intermediate 11

To a solution of Intermediate 10 (5.6 g, 15.7 mmol) in THE (60 mL) was added NaH (0.8 g, 19 mmol) at 0-5° C. under N2, the mixture was stirred at RT for 2 hours. The mixture was poured into ice water, extracted with dichloromethane. The organic phase was washed with brine, dried over Na₂SO₄ and concentrated in vacuum, The crude material was purified by silica gel chromatography to give Intermediate 11 (3.0 g, 56.6%) as a yellow solid.

### Synthesis of Intermediate 12

To a solution of Intermediate 11 (3.0 g, 8.9 mmol) in HCl (6 N, 30 mL) was heated to reflux for 3 hours. The mixture was cooled, poured into ice water, extracted with dichloromethane. The organic phase was washed with brine, dried over Na₂SO₄ and concentrated in vacuum to give a residue, which was purified by silica gel chromatography to give Intermediate 12 (1.8 g, 64.2%) as a yellow solid. ¹H NMR (CDCl₃, 400 MHz): δ (ppm) 15.19 (s, 1H), 8.70 (s, 1H), 7.39-7.44 (m, 1H), 4.42-4.45 (m, 1H), 2.94-2.95 (d, 3H, J=4.0 Hz), 1.90-2.07 (m, 4H), 0.85-0.91 (m, 6H).

### Synthesis of Compound I-3

To a solution of Intermediate 12 (1.0 g. 3.2 mmol) in DMSO (20 mL) was added cyclohexylamine (0.98 g, 9.7 mmol) and Et₃N (0.96 g, 9.7 mmol), then the mixture was stirred at 70° C. for 16 hours. The mixture was poured into ice water, extracted with dichloromethane, and concentrated in vacuum. The crude material was purified by silica gel chromatography to give Compound I-3 (170 mg, 13%) as a white solid. ¹H NMR (CDCl₃, 400 MHz): δ (ppm) 16.23 (s, 1H), 8.57 (s, 1H), 6.46-6.51 (d, 1H, J=7.2 Hz), 4.67 (bs, 1H), 4.37-4.42 (m, 1H), 3.37-3.39 (m, 1H), 2.84-2.85 (d, 3H, J=3.2 Hz), 1.89-2.09 (m, 6H), 1.85-1.87 (m, 2H), 1.83-1.84 (d, 1H, J=4.0 Hz), 1.32-1.41 (m, 5H), 0.87-0.92 (m, 6H); MS: 389.3 [M+1].

### Example 3. Synthesis of Compound I-4, Compound I-5, Compound I-6, and Compound I-7

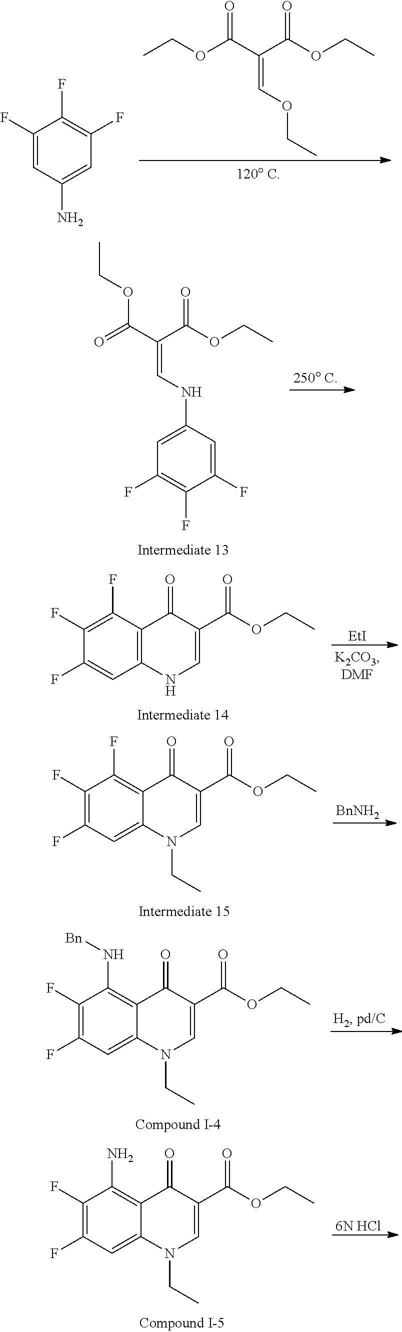

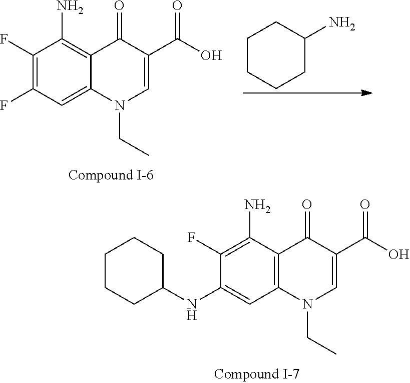

### Synthesis of Intermediate 13

A mixture of 3,4,5-trifluoroaniline (20 g, 0.14 mmol) and diethyl 2-(ethoxymethylene)-malonate (29.4 g, 0.14 mmol) was stirred at 120° C. for 3 hours and cooled to RT. Methyl t-butyl Ether (MTBE) was added and solid filtered off to give Intermediate 13 (20 g, 46%) as an off-white solid. ¹H NMR (CDCl₃, 400 MHz): δ (ppm) 10.94-10.96 (bd, 1H, J=12.8 Hz), 8.28-8.32 (d, 1H, J=13.2 Hz), 6.75-6.78 (m, 2H), 4.23-4.33 (m, 4H), 1.32-1.39 (m, 6H).

### Synthesis of Intermediate 14

A mixture of Intermediate 13 (20 g, 63 mmol) in diphenyl ether (200 mL) was stirred at 250° C. for 3 hours, cooled. Then Methyl t-Butyl Ether (MTBE) was added and the solid filtered to give Intermediate 14 (10 g, 58.8%) as an off-white solid.

### Synthesis of Intermediate 15

To a solution of Intermediate 14 (10 g, 36.9 mmol) in DMF (100 mL) was added K₂CO₃ (15.3 g, 110.7 mmol) and iodoethane (11.5 g, 73.8 mmol). The mixture was stirred at 68° C. for 3 hours. The mixture was poured into ice-water and extracted with EtOAc. The organic phase was washed with brine, dried over Na₂SO₄ and concentrated. The residue was triturated with MTBE and filtered to give Intermediate 15 (7 g, 63%) as a yellow solid. ¹H NMR (CDCl₃, 400 MHz): δ (ppm) 8.39 (s, 1H), 7.01-7.06 (m, 1H), 4.36-4.41 (m, 2H), 4.14-4.19 (m, 2H), 1.52-1.55 (t, 3H, J=7.2 Hz), 1.38-1.42 (t, 3H, J=7.2 Hz).

### Synthesis of Compound I-4

To a solution of Intermediate 15 (7.0 g, 23.1 mmol) in toluene (140 mL) was added benzylamine (3.0 g, 27.7 mmol) and triethylamine (7.0 g, 70.0 mmol). The mixture was stirred at 100° C. for 4 hours. The mixture was poured into ice-water and extracted with dichloromethane. The organic phase was washed with brine, dried over Na₂SO₄ and concentrated. The was triturated with MTBE and the solid filtered off to give Compound I-4 (6 g, 66%) as a yellow solid. ¹H NMR (CDCl₃, 400 MHz): δ (ppm) 11.14 (s, 1H), 8.30 (s, 1H), 7.36-7.38 (m, 2H), 7.25-7.32 (m, 2H), 7.21-7.23 (m, 1H), 6.21-6.26 (m, 1H), 4.70-4.73 (m, 2H), 4.35-4.41 (m, 2H), 4.02-4.07 (m, 2H), 1.46-1.50 (t, 3H, J=7.2 Hz), 1.36-1.40 (t, 3H, J=7.2 Hz).

### Synthesis of Compound I-5

To a solution of Compound I-4 (6.0 g, 15.5 mmol) in EtOH (60 mL) and acetic acid (60 mL) was added Pd/C (4.0 g, 10% wt). The mixture was stirred at 30-40° C. for 4-5 hours under H₂ at 1 atmosphere. The mixture was filtered and concentrated. The residue was triturated with a mixture solution (PE/EA=15/1, 100 mL) and the solid filtered off to give Compound I-5 (4 g, 86.9%) as a yellow solid. ¹H NMR (CDCl₃, 400 MHz): δ (ppm) 8.34 (s, 1H), 6.27-6.32 (m, 1H), 4.37-4.42 (m, 2H), 4.06-4.11 (m, 2H), 1.49-1.50 (t, 3H, J=7.2 Hz), 1.38-1.42 (t, 3H, J=7.2 Hz).

### Synthesis of Compound I-6

A mixture of Compound I-5 (4.0 g, 13.5 mmol) in HCl (6 N, 40 mL) was stirred at 100° C. for 6 hours and cooled to RT. The mixture was filtered to give Compound I-6 (2.3 g, 64%) as a white solid. ¹H NMR (DMSO-d₆, 400 MHz): δ (ppm) 14.78 (s, 1H), 8.90 (s, 1H), 7.84-7.93 (bs, 2H), 7.03-7.08 (m, 1H), 4.39-4.44 (m, 2H), 1.30-1.36 (t, 3H, J=7.2 Hz).

### Synthesis of Compound I-7

To a solution of Compound I-6 (0.5 g, 1.86 mmol) in DMSO (5 mL) was added cyclohexylamine (0.6 g, 5.6 mmol) and triethylamine (0.6 g, 5.6 mmol), then the mixture was stirred at 100° C. for 16 hours. The mixture was poured into ice water, filtered, the filter cake was washed with DMSO/H₂O=1/1. The crude product was recrystallized from EtOH to give Compound I-7 (58 mg, 9.6%) as a yellow solid. ¹H NMR (DMSO-d₆, 400 MHz): δ (ppm) 15.54 (s, 1H), 8.62 (s, 1H), 7.17 (s, 2H), 6.15 (s, 1H), 6.01 (s, 1H), 4.33 (s, 2H), 3.49 (s, 1H), 1.90-1.93 (d, 2H, J=6.0 Hz), 1.72-1.76 (m, 2H), 1.62-1.65 (m, 1H), 1.27-1.44 (m, 7H), 1.14-1.20 (m, 1H); MS: 348.2 [M+1].

### Synthesis of Compound I-8

Compound I-8 synthesized in a similar matter to Compound I-7 using 4-Aminotetrahydropyran and Compound I-6 as the starting materials. MS: 350.1 [M+1].

### Synthesis of Compound I-9

Compound I-9 synthesized in a similar matter to Compound I-7 using cyclopentylamine and Compound I-6 as the starting materials. MS: 334.1 [M+1].

### Synthesis of Compound I-10

Compound I-10 synthesized in a similar matter to Compound I-7 using 2,3-dihydro-1H-Inden-1-amine and Compound I-6 as the starting materials. MS: 382.0 [M+1].

### Synthesis of Compound I-11

Compound I-10 synthesized in a similar matter to Compound I-7 using 2,3-dihydro-1H-Inden-1-amine and Compound I-6 as the starting materials. MS: 382.0 [M+1].

### Synthesis of Compound I-12

Compound I-12 synthesized in a similar matter to Compound I-7 using cyclobutylamine and Compound I-6 as the starting materials. MS: 320.1 [M+1].

### Example 4: Synthesis of Compound I-13 and Compound I-14

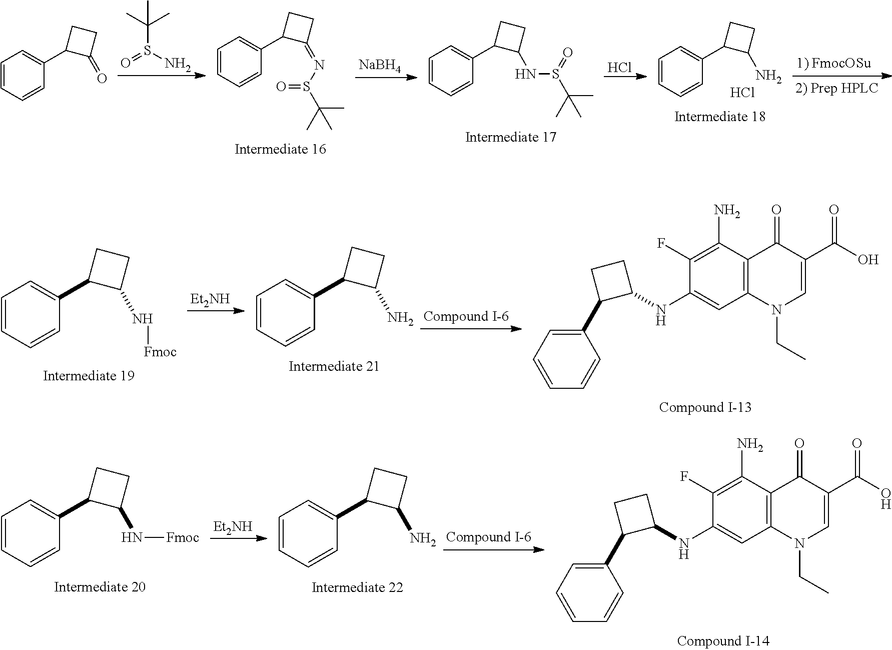

### Synthesis of Intermediate 16

To a solution of 2-phenylcyclobutanone (3.5 g, 23.9 mmol) and Ti(OEt)₄ (11.2 g) in THE (35 mL) was added 2-methylpropane-2-sulfinamide (2.8 g, 23.9 mmol) at RT. Then the reaction mixture is stirred at RT for 3 hours. The reaction mixture was poured into ice-water, extracted with EtOAc (250 mL×3). The combined organic layers was washed with brine, dried over Na₂SO₄ and filtered. The filtration was concentrated in vacuum to give the crude product, which was purified by chromatography to give Intermediate 16 (3.5 g, 58.8%) as light yellow oil.

¹H NMR (CDCl₃, 400 MHz): δ (ppm) 7.24-7.35 (m, 6H), 4.53-4.57 (m, 1H), 3.30-3.35 (m, 2H), 2.56-2.61 (m, 1H), 2.30-2.35 (m, 1H), 1.14-1.28 (m, 9H).

### Synthesis of Intermediate 17

To a solution of Intermediate 16 (4 g, 16 mmol) in THF/MeOH (38 mL/38 mL) was added NaBH₄ (1.2 g, 31.7 mmol) at 0° C. Then the reaction mixture was allowed to warm up to RT for 30 min. The reaction mixture was poured into ice-water, extracted with Dichloromethane (250 mL×3). The combined organic layers was washed with brine, dried over Na₂SO₄ and filtered. The filtration was concentrated to give the crude Intermediate 17 (3.2 g) as light yellow oil.

### Synthesis of Intermediate 18

To a solution of Intermediate 17 (3.2 g, 13 mmol) in MeOH (20 mL) was added MeOH/HCl (20 mL, 8M), then the reaction mixture was stirred at RT for 30 min. The reaction mixture was concentrated to give the crude product Intermediate 18 (2.0 g, 83.3°/o) as a white solid.

1H NMR (DMSO-d₆, 400 MHz): δ (ppm) 8.52 (brs, 2H), 8.04 (brs, 1H), 7.22-7.37 (m, 5H), 3.62-3.93 (m, 2H), 2.59-1.75 (m, 4H).

### Synthesis of Intermediate 19 and Intermediate 20

To a solution of Intermediate 18 (2.0 g, 10.9 mmol) and NaHCO₃ (1.4 g, 16.3 mmol) in CH₃CN/H₂O (100 mL/100 mL) was added 9-Fluorenylmethyl N-succinimidyl carbonate (FmocOSu, 3.6 g, 10.9 mmol). Then the reaction mixture is stirred at RT for 1 hr. The reaction mixture was then poured into ice-water, extracted with EtOAc (200 mL×3). The combined organic layers was washed with brine, dried over Na₂SO₄ and filtered. The filtrate was concentrated to give the crude product, which was purified by chromatography to give a mixture of Intermediate 19 trans relative and Intermediate 20 cis relative (4.8 g) as light yellow solid, then separated by pre-HPLC to give Intermediate 19 trans relative (2.0 g, 40%) and Intermediate 20 cis relative (1.5 g, 30%).

Intermediate 19 ¹H NMR (CDCl₃, 400 MHz): δ (ppm) 7.74-7.76 (m, 2H), 7.43-7.57 (m, 4H), 7.28-7.41 (m, 4H), 7.18-7.23 (m, 3H), 5.02-5.04 (m, 1H), 4.19-4.36 (m, 4H), 3.31-3.36 (m, 1H), 2.13-2.35 (m, 2H), 1.72-1.89 (m, 2H).

Intermediate 20 ¹H NMR (CDCl₃, 400 MHz): δ (ppm) 7.72-7.74 (m, 2H), 7.43-7.45 (m, 6H), 7.25-7.39 (m, 5H), 4.32-4.64 (m, 3H), 4.10-4.13 (m, 1H), 3.88-3.90 (m, 1H), 2.05-2.51 (m, 4H).

### Synthesis of Intermediate 21 Trans Relative

A mixture of Intermediate 19 trans relative (0.9 g) in DCM (9 mL) was added Et₂NH (4.5 mL), the mixture was heated to 34° C. for 5 hrs, the mixture was concentrated, MeOH was added, filtered, the filtration was concentrated to give a crude of Intermediate 21 trans relative (1.0 g, crude) as a yellow oil.

### Synthesis of Compound I-13 Trans Relative

To a solution of Intermediate 21 trans relative (500 mg, crude) in DMSO (8 mL) was added Compound I-6 (161 mg, 0.6 mmol) and Et₃N (240 mg, 2.4 mmol), the mixture was stirred at 100° C. for 24 hrs. The mixture was poured into ice water, filtered to give crude product, which was added into EtOH and stirred at 40° C. for 1 hour, then filtered to give Compound I-13 trans relative (118 mg, 48.1%) as a white solid.

1H NMR (DMSO-d₆, 400 MHz): δ (ppm) 15.46 (s, 1H), 8.58 (s, 1H), 7.23-7.39 (m, 4H), 7.09-7.21 (m, 3H), 7.00-7.02 (m, 1H), 5.65 (d, J=6.8 Hz, 1H), 4.18-4.21 (m, 1H), 3.96-4.16 (m, 1H), 3.62-3.65 (m, 1H), 3.44-3.49 (m, 1H), 2.19-2.37 (m, 1H), 1.88-2.14 (m, 2H), 1.86-1.88 (m, 1H), 1.23 (t, J=6.8 Hz, 3H); MS: 396.1 [M+1].

### Synthesis of Intermediate 22 Cis Relative

A mixture of Intermediate 20 cis relative (0.7 g) in DCM (7 mL) was added Et₂NH (3.5 mL), the mixture was heated to 34° C. for 5 hrs, the mixture was concentrated, MeOH was added, filtered, the filtrate was concentrated to give a crude of Intermediate 22 cis relative (400 mg, crude) as a yellow oil.

### Synthesis of Compound 14 Cis Relative

To a solution of Intermediate 22 cis relative (400 mg, crude) in DMSO (10 mL) was added Compound I-6 (214 mg, 0.8 mmol) and Et₃N (272 mg, 2.7 mmol), the mixture was stirred at 100° C. for 40 hrs. The mixture was poured into ice water, filtered to give crude product, which was added into (DCM/MeOH/hexane=15/1/15) and then filtered to give Compound I-14 cis relative (80 mg, 50°%) as a white solid.

¹H NMR (DMSO-d₆, 400 MHz): δ (ppm) 15.49 (s, 1H), 8.66 (s, 1H), 7.17-7.26 (m, 4H), 7.05-7.11 (m, 3H), 6.35 (brs, 1H), 5.88 (d, J=6.0 Hz, 1H), 4.30-4.55 (m, 3H), 3.94-3.95 (m, 1H), 2.26-2.33 (m, 1H), 2.18-2.21 (m, 1H), 1.37 (t, J=6.8 Hz, 3H); MS: 396.1 [M+1]+.

Synthesis of Compound I-15. Compound I-15 synthesized in a similar matter to Compound I-7 using cyclopropylamine and Compound I-6 as the starting materials. MS: 306.1 [M+1].

### Example 5: Synthesis of Compound I-16

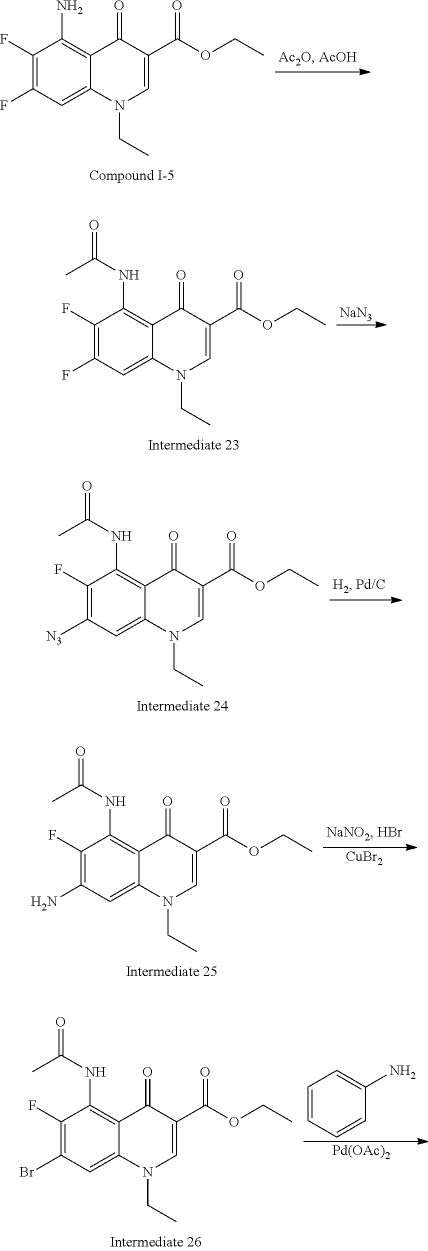

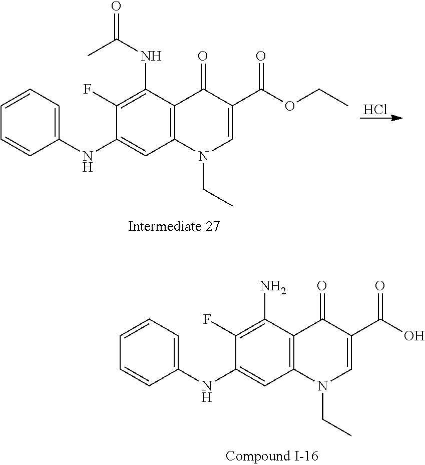 [SMILES: CCOC(=O)c1cn(CC)c2cc(Nc3ccccc3)c(F)c(NC(C)=O)c2c1=O ; Cl ; CCn1cc(C(=O)O)c(=O)c2c(N)c(F)c(Nc3ccccc3)cc21]

### Synthesis of Intermediate 23

To a solution of Compound I-5 (6.0 g) in AcOH (60 mL) was added Ac₂O (6 mL), the mixture was stirred at 100° C. for 16 hrs. The mixture was concentrated, poured into NaHCO₃(aq), extracted with EtOAc, the organic phase was washed with brine, dried over Na₂SO₄ and concentrated in vacuum to give Intermediate 23 (5.0 g, crude) as a yellow oil.

### Synthesis of Intermediate 24

To a solution of Intermediate 23 (5.0 g, 14.5 mmol) in DMF (80 mL) was added NaN₃ (2.8 g, 43.5 mmol), the mixture was stirred at 65° C. for 6 hrs. The mixture was poured into water, extracted with EtOAc, the organic phase was washed with brine, dried over Na₂SO₄ and concentrated in vacuum to give Intermediate 24 (2.8 g, crude) as a yellow oil.

### Synthesis of Intermediate 25

To a solution of Intermediate 24 (2.8 g, crude) in MeOH (55 mL) was added Pd/C (1.0 g, 10% wt), the mixture was stirred at 40° C., 50 psi under H₂ for 1 hrs, filtered, the filtered cake was washed with MeOH/DCM/THF=1/1/1, the filtration was concentrated to give crude, which was added into MTBE/PE=1/1 and stirred for 30 min, filtered to give Intermediate 25 (1.9 g, 73%) as a yellow solid.

¹H NMR (DMSO-d₆, 400 MHz): δ (ppm) 11.68 (s, 1H), 8.51 (s, 1H), 6.72 (d, J=6.8 Hz, 1H), 6.35 (s, 1H), 4.17-4.22 (m, 4H), 2.12 (s, 3H), 1.36 (t, J=6.8 Hz, 3H), 1.27 (t, J=6.8 Hz, 3H).

### Synthesis of Intermediate 26

To a solution of Intermediate 25 (0.9 g, 2.7 mmol) in HBr (80 mL, 5%) was added CuBr₂ (1.9 g, 13.4 mmol), the mixture was cooled to 0° C., NaNO₂ (0.7 g, 5.4 mmol) in water (5 mL) was added, the mixture was stirred at 40° C. for 2 hrs, the mixture was poured into ice-water, extracted with EtOAc, and concentrated to give a crude, which was added into MTBE and stirred for 30 min, filtered to give Intermediate 26 (0.6 g, 50%) as a yellow solid.

¹H NMR (DMSO-d₆, 400 MHz): δ (ppm) 11.38 (s, 1H), 8.69 (s, 1H), 8.03 (d, J=4.8 Hz, 1H), 4.42-4.44 (m, 2H), 4.21-4.26 (m, 2H), 2.16 (s, 3H), 1.26-1.35 (m, 6H).

### Synthesis of Intermediate 27

To a solution of Intermediate 26 (1.0 g, 2.5 mmol), Aniline (353 mg, 3.8 mmol) in dioxane (20 mL) was added Pd(OAc)₂ (180 mg, 0.8 mmol) and xantphos (636 mg, 1.1 mmol) under N2. Then Cs₂CO₃ (1.2 g, 3.8 mmol) was added, the mixture was stirred at 100° C. under N2 for 2 hrs, the mixture was poured into ice-water, extracted with EtOAc, and concentrated to give a crude, which was purified by silica gel chromatography to give Intermediate 27 (0.5 g, 52.6%) as a yellow solid.

¹H NMR (DMSO-d₆, 400 MHz): δ (ppm) 11.63 (s, 1H), 8.86 (s, 1H), 8.57 (s, 1H), 7.32-7.40 (m, 4H), 7.00-7.10 (m, 2H), 4.18-4.24 (m, 4H), 2.16 (s, 3H), 1.26-1.31 (m, 6H).

### Synthesis of Compound I-16

A mixture of Intermediate 27 (0.5 g, 1.2 mmol) in HCl (6N, 5 mL) and dioxane (5 mL) was heated at 100° C. for 4 hrs, cooled and filtered to give Compound I-16 (138 mg, 33%) as a yellow solid.

¹H NMR (DMSO-d₆, 400 MHz): δ (ppm) 15.32 (s, 1H), 8.74 (s, 2H), 7.35-7.39 (m, 4H), 7.23-7.29 (m, 2H), 7.06-7.10 (m, 1H), 6.43 (d, J=6.4 Hz, 1H), 4.20-4.22 (m, 2H), 1.30 (t, J=6.8 Hz, 3H); MS: 342.1 [M+1].

Synthesis of Compound I-17. Compound I-17 synthesized in a similar matter to Compound I-7 using benzylamine and Compound I-6 as the starting materials. MS: 356.0 [M+1].

Synthesis of Compound I-18. Compound I-18 synthesized in a similar matter to Compound I-7 using Benzeneethanamine and Compound I-6 as the starting materials. MS: 370.0 [M+1].

Synthesis of Compound I-19. Compound I-19 synthesized in a similar matter to Compound I-7 using Benzenepropanamine and Compound I-6 as the starting materials. MS: 384.0 [M+1].

### Synthesis of Compound I-20

To a solution of Compound I-6 (0.5 g, 1.9 mmol) in DMSO (5 mL) was added triethylamine (0.6 g, 5.7 mmol), then trans-2-phenylcyclopropylamine (0.8 g, 5.7 mmol) was added. The mixture was stirred at 100° C. for 16 hours, cooled. Then the mixture was poured into ice-water, filtered, the filtered cake was recrystallized from EtOH to give Compound I-20 (62 mg, 9.3%) as a yellow solid. ¹H NMR (DMSO-d₆, 400 MHz): δ (ppm) 15.44 (s, 1H), 8.56 (s, 1H), 7.29-7.33 (m, 3H), 7.21-7.25 (m, 4H), 5.95-5.97 (d, 1H, J=6.0 Hz), 3.94-3.98 (t, 2H, J=6.8 Hz), 2.67 (s, 1H), 1.97-2.00 (m, 1H), 1.44-1.48 (m, 1H), 1.33-1.38 (m, 1H), 0.92-0.96 (t, 3H, J=6.8 Hz); MS: 382.4 [M+1].

### Synthesis of Compound I-21

Compound I-21 synthesized in a similar matter to Compound I-20 using (1R,2S)-2-phenylcyclopropan-1-amine and Compound I-6 as the starting materials. MS: 382.4 [M+1].

### Synthesis of Compound I-22

Compound I-22 synthesized in a similar matter to Compound I-20 using (1 S,2R)-2-phenylcyclopropan-1-amine and Compound I-6 as the starting materials. MS: 382.4 [M+1].

### Synthesis of Compound I-23

Compound I-23 synthesized in a similar matter to Compound I-20 using cis-2-phenylcyclopropylamine and Compound I-6 as the starting materials. MS: 382.4 [M+1].

### Synthesis of Compound I-26

To a solution of Compound I-6 (1.8 g, 6.7 mmol) in DMSO (18 mL) was added triethylamine (0.7 g, 6.5 mmol), then {N-[relative-(1R,2S)-2-phenyl]cyclopropyl]-4-Piperidinamine} (1.4 g, 6.5 mmol) was added. The mixture was stirred at 60° C. for 16 hours, cooled. Then the mixture was poured into ice-water, filtered, the filtered cake was purified by pre-TLC to give Compound I-26 (198 mg, 19.2%) as a yellow solid.

¹H NMR (CDCl₃, 400 MHz): δ (ppm) 15.09 (s, 1H), 8.52 (s, 1H), 7.25-7.28 (m, 2H), 7.15-7.18 (m, 1H), 7.03-7.05 (d, 2H, J=7.2 Hz), 6.59-6.64 (m, 2H), 6.04-6.05 (d, 1H, J=5.6 Hz), 4.13-4.20 (m, 2H), 3.64-3.68 (d, 2H, J=12.4 Hz), 2.84-2.96 (m, 3H), 2.35-2.61 (m, 1H), 2.03-2.09 (m, 2H), 1.89-1.93 (m, 1H), 1.51-1.65 (m, 6H), 1.01-1.13 (m, 2H); MS: 465.3 [M+1].

### Example 6. Synthesis of Compound I-27 and Compound I-28

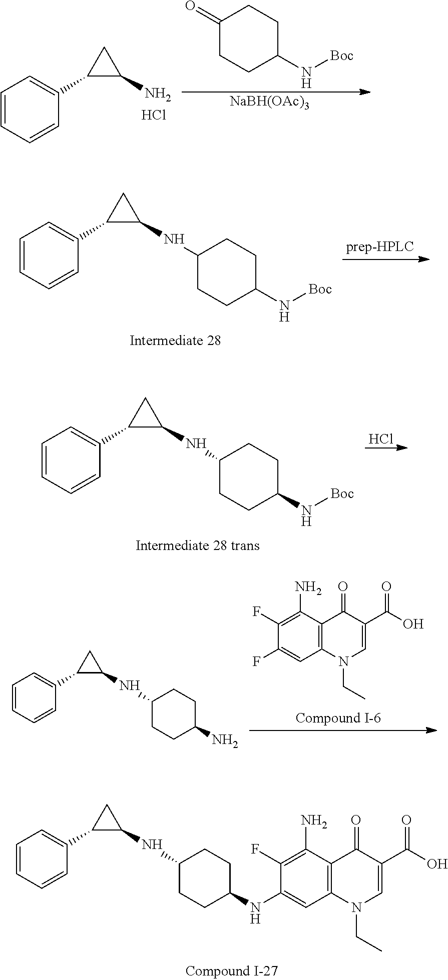

### Synthesis of Intermediate 28

To a solution of tranylcypromine trans relative (3.0 g, 17.8 mmol) in DCE (90 mL) was added N-4-Boc-aminocyclohexanone (4.5 g, 21.0 mmo) under N₂ at 0° C. the mixture was stirred at 0° C. for 1 hr. AcOH (1.3 g, 21.0 mmol) was added, then NaBH(OAc)₃ (6.8 g, 32.0 mmo) was added slowly. The mixture was stirred at 30° C. for 16 hrs, and then poured into ice water, extracted with EtOAc, the organic phase was concentrated in vacuum, purified by silica gel of chromatography to give crude Intermediate 28 cyclopropyl trans relative (7.5 g) as yellow oil.

### Synthesis of Intermediate 28 Trans Cyclohexyl Cyclopropyl Trans Relative

Crude of Intermediate 28 cyclopropyl trans relative (7.5 g) was separated by prep-HPLC to give Intermediate 28 trans cyclohexyl cyclopropyl trans relative (1.0 g, 34.4%) as white solid.

¹HNMR (CDCl₃, 400 MHz): δ (ppm) 7.22-7.26 (m, 2H), 7.12-7.16 (m, 1H), 7.01 (d, J=7.2 Hz, 2H), 4.38 (brs, 1H), 3.40 (brs, 1H), 2.55-2.58 (m, 1H), 2.28-2.32 (m, 1H), 1.98-2.01 (m, 4H), 1.83-1.86 (m, 1H), 1.43 (s, 9H), 0.93-1.22 (m, 6H).

### Synthesis of Intermediate 29 Trans Cyclohexyl Cyclopropyl Trans Relative

To a solution of Intermediate 28 trans cyclohexyl cyclopropyl trans relative (1.0 g, 3.0 mmol) in DCM (10 mL) was added HCl/dioxane (10 mL, 7 mol/L), the mixture was stirred at RT for 2 hrs. The mixture was concentrated, ice water was added, adjusted pH=8˜9 with Na₂CO₃ (aq), extracted with DCM/MeOH=10/1, the organic phase was dried over Na₂SO₄ and concentrated in vacuum to give crude Intermediate 29 trans cyclohexyl cyclopropyl trans relative (0.6 g, 85%) as yellow oil.

### Synthesis of Compound I-27 Trans Cyclohexyl Cyclopropyl Trans Relative

To a solution of Intermediate 29 trans cyclohexyl cyclopropyl trans relative (0.6 g, 2.6 mmol) in DMSO (8 mL) was added Et₃N (0.3 g, 2.6 mmol), then Compound I-6 (0.3 g, 0.9 mmol) was added. The mixture was stirred at 75° C. for 16 hrs, cooled. The mixture was poured into ice-water, filtered, the crude was purified by pre-HPLC to give product Compound I-27 trans cyclohexyl cyclopropyl trans relative (33 mg, 8%) as yellow solid.

¹HNMR (DMSO-d₆, 400 MHz): δ (ppm) 8.66 (s, 1H), 7.20-7.27 (m, 5H), 7.01-7.15 (m, 2H), 6.23 (d, J=7.2 Hz, 2H), 6.03 (d, J=7.2 Hz, 2H), 4.35-4.76 (m, 2H), 3.49-3.50 (m, 1H), 2.23-2.24 (m, 1H), 1.85-1.92 (m, 4H), 1.75-1.76 (m, 1H), 1.27-1.36 (m, 7H), 0.9-0.97 (m, 2H); MS: 479.2 [M+1].

### Synthesis of Compound I-28

Intermediate 28 (2.5 g) was separated by pre-HPLC, to give Intermediate 28 trans cyclohexyl cyclopropyl trans relative (1.0 g, 40%) and Intermediate 28 cis cyclohexyl cyclopropyl trans relative (1.0 g, 40%). Intermediate 28 cis cyclohexyl cyclopropyl trans relative was deprotected as described in the synthesis of Intermediate 29. To a solution of Compound I-6 (0.8 g, 3.0 mmol) in DMSO (15 mL) was added triethylamine (0.44 g, 4.34 mmol), then Intermediate 29 cis cyclohexyl cyclopropyl trans relative (1.0 g, 4.34 mmol) was added. The mixture was stirred at 70° C. for 16 hours, cooled. Then the mixture was poured into ice-water, filtered, and the crude material was purified by prep HPLC to give Compound I-28 cis cyclohexyl cyclopropyl trans relative (57 mg, 8.1%) as a yellow solid.

¹H NMR (CDCl₃, 400 MHz): δ (ppm) 15.40 (s, 1H), 8.48 (s, 1H), 7.23-7.26 (m, 2H), 7.14-7.17 (m, 1H), 7.01-7.13 (d, 2H, J=7.6 Hz), 6.45-6.52 (m, 2H), 5.81-5.82 (d, 1H, J=4.4 Hz), 4.63 (s, 1H), 4.13-4.14 (m, 2H), 3.60 (m, 1H), 2.88 (s, 1H), 2.77 (s, 1H), 1.86-1.77 (m, 7H), 1.51-1.61 (m, 3H), 1.25-1.33 (m, 3H), 0.83-0.88 (m, 2H); MS: 479.2 [M+1].

### Example 7. Synthesis of Compound I-29

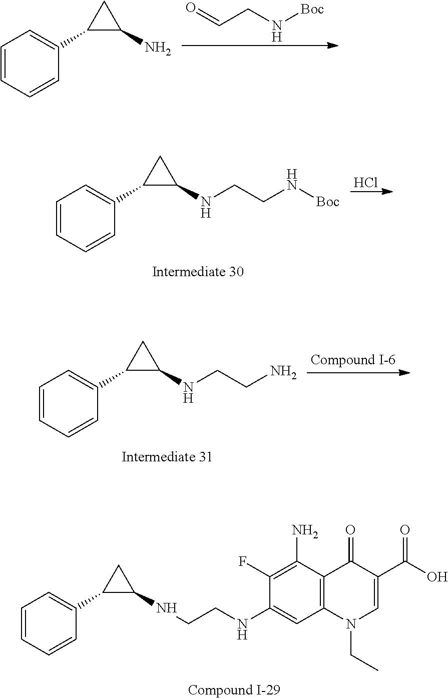 [SMILES: N[C@@H]1C[C@H]1c1ccccc1 ; CC(C)(C)OC(=O)NCC=O ; CC(C)(C)OC(=O)NCCN[C@@H]1C[C@H]1c1ccccc1 ; Cl ; NCCN[C@@H]1C[C@H]1c1ccccc1 ; CCn1cc(C(=O)O)c(=O)c2c(N)c(F)c(NCCN[C@@H]3C[C@H]3c3ccccc3)cc21]

### Synthesis of Intermediate 30

To a solution of tranylcypromine trans relative (2.5 g, 18.8 mmol) in dichloromethane (75 mL) was added N-(2-oxoethyl)-Carbamic acid-1,1-dimethylethyl ester (3.0 g, 18.8 mmol) under N₂ at 0° C., the mixture was stirred at 0° C. for 1 hour. AcOH (1.1 g, 18.8 mmol) was added, then NaBH(Ac)₃ (7.2 g, 33.8 mmol) was added slowly. The mixture was stirred at 0-10° C. for 2 hours poured into ice-water and extracted with dichloromethane. The organic phase was concentrated in vacuum, purified by silica gel chromatography to give crude Intermediate 30 trans relative (1.0 g, crude) as a yellow oil.

### Synthesis of Intermediate 31

To a solution of Intermediate 30 trans relative (2.0 g, crude) in dioxane (20 mL) was added HCl/dioxane (20 mL, 4 mol/L), the mixture was stirred at rt for 2 hours. The mixture was concentrated, ice water was added, adjusted PH=8-9, extracted with dichloromethane-/MeOH=10/1, the organic phase was washed brine, dried over Na₂SO₄ and concentrated in vacuum to give crude Intermediate 31 (0.6 g crude, 90%) as a yellow oil.

### Synthesis of Compound I-29

To a solution of Intermediate 31 trans relative (1.2 g, crude, 6.8 mmol) in DMSO (18 mL) was added Et₃N (0.7 g, 6.8 mmol), then Compound I-6 (0.6 g, 2.3 mmol). The mixture was stirred at 70° C. for 16 hours and cooled. The mixture was poured into ice-water and filtered. The crude material was purified by prep HPLC to give Compound I-29 trans relative (70 mg, 3.6%) as a white solid.

¹H NMR (CDCl₃, 400 MHz): δ (ppm) 15.42 (s, 1H), 8.48 (s, 1H), 7.21-7.29 (m, 2H), 7.13-7.17 (m, 1H), 6.97-6.99 (d, 2H, J=7.2 Hz), 6.59-6.62 (m, 2H), 5.80-5.82 (d, 1H, J=6.4 Hz), 5.04 (s, 1H), 4.07-4.13 (m, 2H), 3.28-3.34 (m, 2H), 3.10-3.16 (m, 2H), 2.33-2.35 (m, 1H), 1.86-1.88 (m, 1H), 1.25-1.54 (m, 3H), 1.08-1.12 (m, 1H), 1.02-1.05 (m, 1H); MS: 424.9 [M+1].

### Example 8. Synthesis of Compound I-29

Synthetic route:

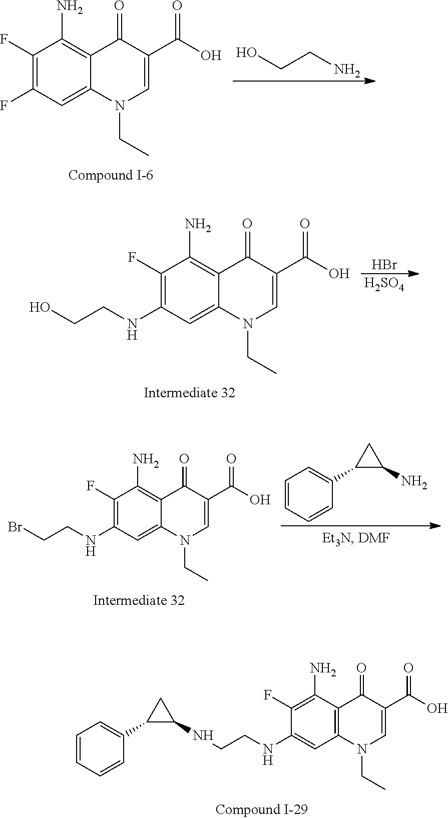

### Synthesis of Intermediate 32

To a solution of Compound I-6 (6.0 g, 22.0 mmol) in DMSO (60 mL) was added Et₃N (6.7 g, 66.0 mmol), then 2-aminoethanol (4.0 g, 66.0 mmol) was added. The mixture was stirred at 90° C. for 8 hrs, cooled. Then the mixture was poured into ice-water, adjusted pH=4-5, filtered to give Intermediate 32 (5.4 g, 78%) as a yellow solid.

¹H NMR (DMSO-d₆, 400 MHz): δ (ppm) 15.56 (s, 1H), 8.67 (s, 1H), 7.0-7.3 (bs, 2H), 6.46 (1H), 6.08-6.10 (m, 1H), 4.83 (t, J=5.6 Hz, 1H), 4.33-4.38 (m, 2H), 3.58-3.62 (m, 2H), 3.31-3.32 (m, 2H), 1.35 (t, J=7.2 Hz, 3H).

### Synthesis of Intermediate 33

To a solution of Intermediate 32 (5.4 g, 17.4 mmol) in HBr (48%, 78.3 g, 470 mmol) was added H₂SO₄ (17 g, 174 mmol) at 0-10° C. for 16 hrs. Then the mixture was heated to reflux for 8 hrs, cooled, poured into ice-water, filtered to give Intermediate 33 (3.5 g, 53.8%) as a yellow solid.

¹H NMR (DMSO-d₆, 400 MHz): δ (ppm) 15.50 (s, 1H), 8.69 (s, 1H), 7.0-7.4 (bs, 2H), 6.78-6.79 (m, 1H), 6.11-6.12 (d, 1H), 4.36-4.42 (m, 2H), 3.71-3.75 (m, 2H), 3.64-3.67 (m, 2H), 1.35 (t, J=6.8 Hz, 3H).

### Synthesis of Compound I-29

To a solution of Intermediate 33 (0.3 g, 0.8 mmol) in DMF (10 mL) was added Et₃N (0.3 g, 2.4 mmol), then relative Trans-2-phenylcyclopropanamine (0.3 g, 2.4 mmol) was added. The mixture was stirred at 60° C. for 8 hrs, cooled. Then the mixture was poured into ice-water, extracted with DCM/MeOH=10/1, dried with Na₂SO₄, filtered, the crude was purified by prep HPLC to give product Compound I-29 trans relative (0.12 g, 35.0%) as a white solid.

¹H NMR (CDCl₃, 400 MHz): δ (ppm) 15.41 (bs, 1H), 8.47 (s, 1H), 7.04-7.24 (m, 2H), 6.97-7.04 (m, 1H). 6.88-6.90 (m, 2H), 6.44-6.46 (m, 2H), 5.81 (d, J=3.2 Hz, 1H). 5.06 (s, 1H), 4.08-4.11 (m, 2H), 3.10-3.32 (m, 2H), 2.91-3.04 (m, 2H), 2.29-2.34 (m, 1H). 1.85-1.87 (m, 1H), 1.31-1.40 (m, 3H). 1.08-1.12 (m, 1H), 1.02-1.05 (m, 1H); MS: 424.9 [M+1].

### Example 9: Synthesis of Compound I-32

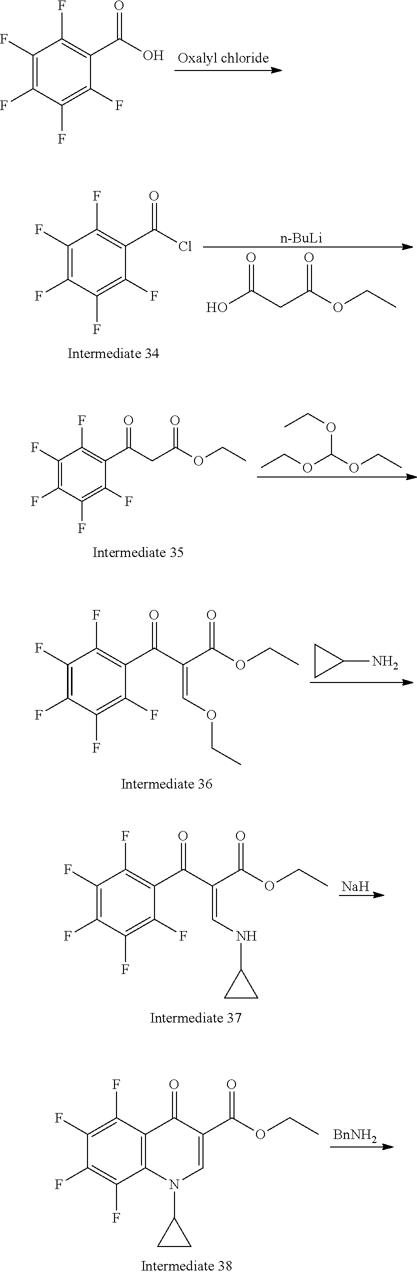

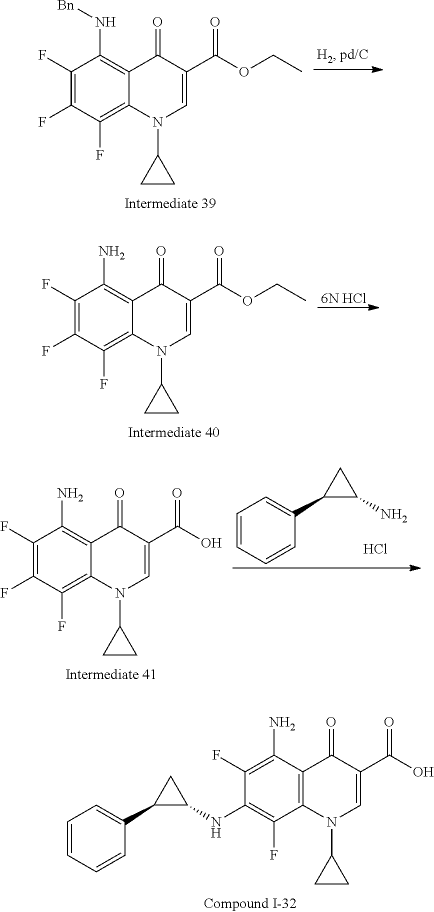

### Synthesis of Intermediate 34

To a solution of 2,3,4,5,6-pentafluorobenzoic acid (20 g, 94.3 mmol) in DCM (200 mL) was added oxalyl chloride (14.4 g, 113.2 mmol) at 0-10° C., DMF (40 drops, cat) was added dropwise. The mixture was stirred at 30° C. for 3 hrs. The mixture was concentrated in vacuum to give Intermediate 34 (22 g, crude) as a yellow oil.

### Synthesis of Intermediate 35

To a solution of mono-Ethyl malonate (25.4 g, 192 mmol) in THE (220 mL) was added n-BuLi (2.2 M, 174 mL) at −10-0° C. under N₂, the mixture was stirred at RT for 1 hr.

Then the mixture was cooled to −78° C. and added Intermediate 34 (22 g, crude) in THE (150 mL). The solution was warmed to RT and stirred for 1 hour. Then NH₄Cl(aq) was added to the solution, extracted with EA, concentrated in vacuum and purified by silica gel chromatography to give Intermediate 35 (10 g, 37.6% for two steps) as a yellow oil.

### Synthesis of Intermediate 36

A mixture of Intermediate 35 (12 g, 42.6 mmol) in triethoxymethane (36 mL) was heated to 145° C. for 2.5 hrs under N₂, then the solution was cooled to RT. The mixture was concentrated in vacuum, toluene was added, and concentrated under vacuum to give crude Intermediate 36 (14 g, crude) as a yellow oil.

### Synthesis of Intermediate 37

To a solution of Intermediate 36 (14 g, crude) in MeOH (120 mL) was added cyclopropanamine (4.7 g, 82 mmol) at 0-10° C., then the mixture was stirred at 28° C. for 1 hour. The mixture was concentrated in vacuum to give a residue, which was purified by silica gel chromatography to give Intermediate 37 (7.2 g, 48.5% for two steps) as a yellow solid.

¹H NMR (CDCl₃, 400 MHz): δ (ppm) 11.11 (brs, 1H), 8.30 (d, J=14.0 Hz, 1H), 4.07 (q, J=7.2 Hz, 2H), 3.01-3.05 (m, 1H), 1.13 (t, J=7.2 Hz, 3H), 0.86-0.99 (m, 4H); MS: 350.1 [M+1].

### Synthesis of Intermediate 38

To a solution of Intermediate 37 (7.2 g, 20.6 mmol) in THE (60 mL) was added NaH (60%, 0.9 g, 22.5 mmol) at 0-5° C. under N₂, the mixture was stirred at RT for 1 hr. The mixture was poured into ice-water, filtered. The filter cake was dried in vacuo to give Intermediate 38 (6.0 g, 88.4%) as a yellow solid.

¹H NMR (CDCl₃, 400 MHz): δ (ppm) 8.49 (s, 1H), 4.38 (q, J=7.2 Hz, 2H), 3.87-3.88 (m, 1H), 1.39 (t, J=7.2 Hz, 3H), 1.11-1.29 (m, 4H); MS: 330.0 [M+1].

### Synthesis of Intermediate 39

To a solution of Intermediate 38 (6.0 g, 18.2 mmol) in toluene (120 mL) was added phenylmethanamine (2.3 g, 21.5 mmol) and triethylamine (5.5 g, 55 mmol). The mixture was stirred at 100° C. for 4 hrs. TLC (DCM/MeOH=20/1) showed the reaction was complete. Then the mixture was poured into ice-water, extracted with DCM, the organic phase was washed with brine, dried over Na₂SO₄ and concentrated to give a residue, which was triturated with MTBE, filtered to give Intermediate 39 (4.5 g, 59.2%) as a yellow solid.

¹H NMR (CDCl₃, 400 MHz): δ (ppm) 10.72 (brs, 1H), 8.40 (s, 1H), 7.22-7.35 (m, 5H), 4.64 (dd, J=4.4 Hz, 6.0 Hz, 2H), 4.36 (q, J=7.2 Hz, 2H), 3.83-3.84 (m, 1H), 1.38 (t, J=7.2 Hz, 3H), 1.17-1.20 (m, 2H), 1.03-1.06 (m, 2H); MS: 417.1 [M+1].

### Synthesis of Intermediate 40

To a solution of Intermediate 39 (4.0 g, 9.6 mmol) in EtOH (40 mL) and acetic acid (40 mL) was added Pd/C (1.0 g, 10% wt). The mixture was stirred at 30-40° C. for 4-5 hrs under H₂ at 1 atm. TLC (DCM/MeOH=20/1) showed the reaction was complete. Then the mixture was filtered, concentrated to give a crude, which was triturated with (PE/EA=15/1), filtered to give Intermediate 40 (2.0 g, 63.9%) as a white solid.

¹H NMR (CDCl₃, 400 MHz): δ (ppm) 8.36 (s, 1H), 6.95 (br, 2H), 4.30 (q, J=7.2 Hz, 2H), 3.76-3.78 (m, 1H), 1.32 (t, J=7.2 Hz, 3H), 1.12-1.15 (m, 2H), 1.00-1.03 (m, 2H); MS: 327.0 [M+1].

### Synthesis of Intermediate 41

A mixture of Intermediate 40 (1.7 g, 5.2 mmol) in HCl (6 N, 17 mL) was stirred at 100° C. for 6 hrs, cooled. Then the mixture was filtered to give Intermediate 41 (1.0 g, 64.3%) as a yellow solid.

¹H NMR (DMSO-d₆, 400 MHz): δ (ppm) 14.36 (s, 1H), 8.63 (s, 1H), 7.78 (br, 2H), 4.09-4.11 (m, 1H), 1.19-1.23 (m, 4H); MS: 299.0 [M+1].

### Synthesis of Compound I-32

To a solution of Intermediate 41 (1.0 g, 3.36 mmol) in DMSO (27 mL) was added tranylcypromine trans relative (1.7 g, 10.1 mmol) and Et₃N (10 g, 10.1 mmol), then the mixture was stirred at 110° C. for 5 hrs. The mixture was poured into ice water, extracted with DCM/MeOH=10:1, concentrated in vacuum to give a residue, which was washed with MTBE and purified by prep. TLC to give Compound I-32 trans relative (195 mg, 14.1%) as a yellow solid.

¹H NMR (DMSO-d₆, 400 MHz): δ (ppm) 14.97 (s, 1H), 8.40 (s, 1H), 7.20-7.40 (m, 2H), 7.12-7.26 (m, 5H), 6.83 (s, 1H), 3.92-3.97 (bm, 1H), 3.10-3.20 (m, 1H), 2.10-2.20 (m, 1H), 1.46-1.48 (m, 1H), 1.20-1.28 (m, 1H), 0.97-1.13 (m, 3H), 0.90-0.96 (m, 1H); MS: 411.9 [M+1].

### Example 10: Synthesis of Compound I-38

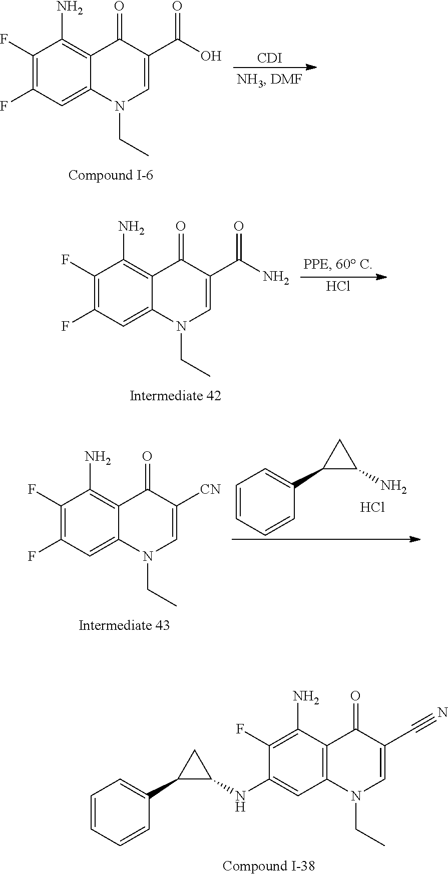

### Synthesis of Intermediate 42

To a solution of Compound I-6 (6.0 g, 22.4 mol) in DMF (66 mL) was added Carbonyl Diimidazole (5.4 g, 33.6 mol). The mixture was warm up to 100° C. for 4 hrs. The reaction was cooled and poured into water and the filtered to give an off-white solid (5.0 g crude). The off-white solid was dissolved into DMF (100 mL) at 25-35° C., then anhydrous NH₃ was bubbled though this solution at 25˜35° C. for 30 min, then the mixture was stirred for another 2 hrs, poured into water, filtered to give Intermediate 42 (3.0 g, 50%) as a white solid.

1H NMR (DMSO-d₆, 400 MHz): δ (ppm) 9.03 (brs, 1H), 8.71 (s, 1H), 7.43 (brs, 1H), 6.88 (dd, J=6.4, 12.8 Hz, 1H), 4.31 (q, J=7.2 Hz, 2H), 1.32 (t, J=7.2 Hz, 3H).

### Synthesis of Intermediate 43

To a solution of Intermediate 42 (3.0 g, 11.1 mol) and Polyphosphate Ester (PPE) (30 g) was warm up to 65° C. for 4 hrs, the mixture was cooled and HCl (1 N, 100 mL) was added, the mixture was stirred for 1 hr, filtered, the filtered cake was purified by silica gel chromatography (DCM/MeOH=200/1˜50/1) to give Intermediate 43 (1.0 g, 35.7%) as a white solid.

¹H NMR (DMSO-d₆, 400 MHz): δ (ppm) 8.70 (s, 1H), 7.92 (br s, 1H), 6.89 (dd, J=6.4, 13.2 Hz, 1H), 4.20 (q, J=7.2 Hz, 2H), 1.31 (t, J=7.2 Hz, 3H).

### Synthesis of Compound I-38

To a solution of Intermediate 43 (0.4 g, 1.6 mmol) in DMSO (10 mL) was added Tranylcypromine trans relative (0.7 g, 5.6 mmol) and Et₃N (0.6 g, 5.6 mmol), the mixture was stirred at 100° C. for 16 hrs. The mixture was poured into ice water, filtered to give crude product, which was purified by silica gel chromatography (DCM/MeOH=200/1˜250/1) and then washed with MTBE to give Compound I-38 trans relative (108 mg, 18%) as a white solid.

¹H NMR (DMSO-d₆, 400 MHz): δ (ppm) 8.44 (s, 1H), 7.28-7.32 (m, 3H), 7.19-7.21 (m, 4H), 7.14 (s, 1H), 5.86 (d, J=6.8 Hz, 1H), 3.75 (q, J=7.2 Hz, 2H), 2.63-2.64 (m, 1H), 1.94-1.98 (m, 1H), 1.41-1.46 (m, 1H), 1.32-1.36 (m, 1H), 0.91 (t, J=7.2 Hz, 3H); MS: 363.1 [M+1].

### Example 11: Synthesis of Compound I-50

Synthetic route:

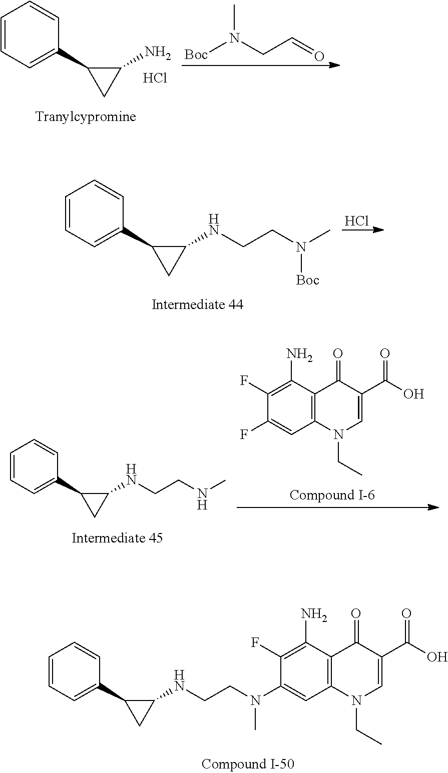

### Synthesis of Intermediate 44

To a solution of tranylcypromine trans relative HCl (3.0 g, 17.8 mmol) in DCE (90 mL) was added tert-butyl methyl(2-oxoethyl)carbamate (3.0 g, 17.8 mmol) under N₂ at 0° C., AcOH (1.0 g, 17.8 mmol) was added. The mixture was stirred at RT for 1 hour. Then NaBH(OAc)₃ (4.5 g, 23.1 mmol) was added slowly at 0° C., the mixture was stirred at 30° C. for 6 hrs, and then poured into ice water, NaHCO₃(aq) was added until pH=8-9, extracted with EtOAc, the organic phase was concentrated in vacuum, purified by pre-HPLC to give Intermediate 44 trans relative (1.5 g, 29.4%) as a yellow oil.

¹H NMR (CDCl₃, 400 MHz): δ (ppm) 7.22-7.26 (m, 2H), 7.12-7.16 (m, 1H), 7.02 (d, J=7.6 Hz, 2H), 3.33-3.38 (m, 2H), 2.86-2.89 (m, 5H), 2.39 (s, 1H), 1.86-1.90 (m, 1H) 1.46 (s, 9H), 1.03-1.07 (m, 1H), 0.95-0.99 (m, 1H).

### Synthesis of Intermediate 45

To a solution of Intermediate 44 trans relative (1.5 g, 5.2 mmol) in DCM (15 mL) was added HCl/dioxane (15 mL, 12 mol/L), the mixture was stirred at RT for 2 hrs. The mixture was concentrated, ice water was added, adjusted pH=8-9 with Na₂CO₃ (aq), extracted with DCM/MeOH=10/1, the organic phase was dried over Na₂SO₄ and concentrated in vacuum to give Intermediate 45 trans relative (0.9 g, 91%) as a yellow oil.

¹H NMR (DMSO-d₆, 400 MHz): δ (ppm) 10.16 (s, 2H), 9.38 (s, 2H), 7.17-7.33 (m, 5H), 3.40-3.42 (m, 3H), 3.31-3.32 (m, 1H), 3.05-3.07 (m, 1H), 2.57-2.60 (m, 4H), 1.58-1.64 (m, 1H), 1.26-1.31 (m, 1H).

### Synthesis of Compound I-50

To a solution of Intermediate 45 trans relative (0.57 g, 3.0 mmol) in DMSO (15 mL) was added triethylamine (0.3 g, 3.0 mmol), then Compound I-6 (0.27 g, 1.0 mmol) was added. The mixture was stirred at 75° C. for 16 hrs. The mixture was poured into ice-water, filtered, the filtered cake was purified by pre-TLC to give product Compound I-50 trans relative (53 mg, 12.1%) as a yellow solid.

¹H NMR (DMSO-d₆, 400 MHz): δ (ppm) 15.21 (s, 1H), 9.05 (s, 2H), 8.77 (s, 1H), 7.27-7.31 (m, 4H), 7.21-7.23 (m, 1H), 7.15-7.20 (m, 2H), 6.17 (m, J=7.2 Hz, 1H), 4.38-4.43 (m, 2H), 3.61-3.63 (m, 2H), 3.38 (s, 2H), 3.03 (s, 4H), 1.47-1.49 (m, 1H), 1.22-1.38 (m, 4H).

### Synthesis of Compound I-52

To a solution of Compound I-6 (2.0 g, 7.5 mmol) in NaOH (aq) (2N, 30 mL) was added Phenylethyl alcohol (2.8 g, 22.5 mmol), the mixture was stirred at 100° C. for 30 hrs. The mixture was poured into ice water, the pH of the solution adjusted to 4 with AcOH, the mixture was filtered, the filtered cake washed with water, then dried to give crude, which was purified by prep-HPLC to give Compound I-52 (144 mg, 5.2%) as a white solid.

¹H NMR (DMSO-d₆, 400 MHz): δ (ppm) 15.11 (s, 1H), 8.81 (s, 1H) 7.31-7.37 (m, 6H), 7.24-7.26 (m, 1H), 6.55 (d, J=6.8 Hz, 1H), 4.43-4.47 (m, 4H), 3.12 (t, J=6.8 Hz, 2H), 1.36 (t, J=6.8 Hz, 3H); MS: 370.9 [M+1].

### Example 12: Synthesis of Compound I-56

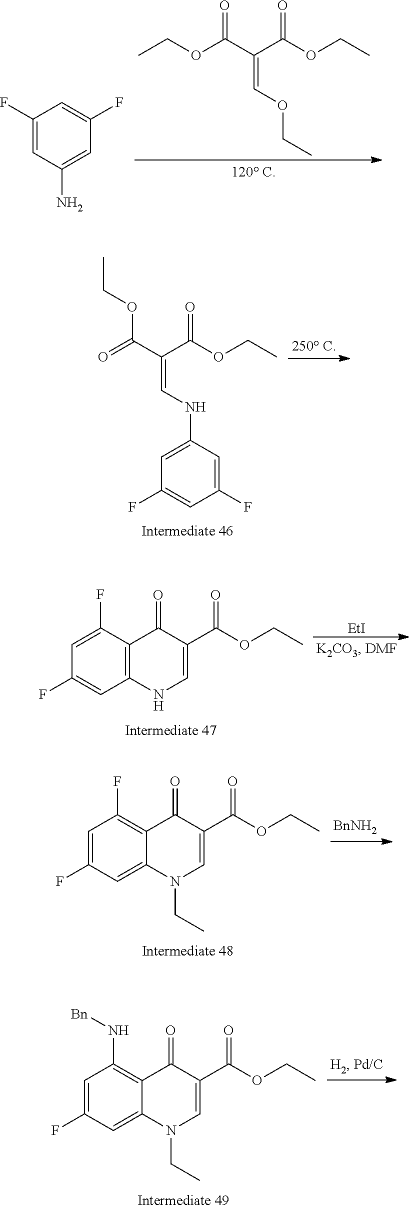

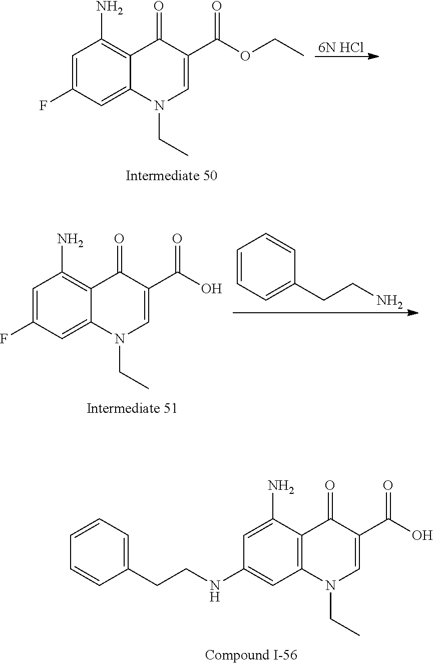

### Synthesis of Intermediate 46

A mixture of 3,5-difluoroaniline (25 g, 0.194 mol) and diethyl 2-(ethoxymethylene) malonate (42 g, 0.194 mol) was stirred at 120° C. for 3 hrs and cooled. MTBE was added, filtered to give Intermediate 46 (25 g, 43%) as a white solid.

¹H NMR (DMSO-d₆, 400 MHz): δ (ppm) 10.62 (s, 1H), 8.33 (s, 1H), 7.25-7.27 (m, 2H), 6.97-6.98 (m, 1H), 4.13-4.22 (m, 4H), 1.24-1.27 (m, 6H).

### Synthesis of Intermediate 47

A mixture of Intermediate 46 (25 g, 84 mmol) in diphenyl ether (62 mL) was stirred at 250° C. for 3 hrs, cooled. Then MTBE was added, filtered to give Intermediate 47 (13 g, 61%) as a white solid.

¹H NMR (DMSO-d₆, 400 MHz): δ (ppm) 12.32 (br s, 1H), 8.49 (s, 1H), 7.15-7.22 (m, 2H), 4.20 (q, J=7.2 Hz, 2H), 1.27 (t, J=7.2 Hz, 3H).

### Synthesis of Intermediate 48

To a solution of Intermediate 47 (13 g, 51 mmol) in DMF (130 mL) was added K₂CO₃ (21.1 g, 153 mmol) and iodoethane (15.9 g, 102 mmol). The mixture was stirred at 68° C. for 3 hrs. The mixture was poured into ice-water, filtered to give the crude solid, which was triturated with MTBE, filtered to give Intermediate 48 (8.5 g, 59.4%) as a yellow solid.

¹H NMR (CDCl₃, 400 MHz): δ (ppm) 8.38 (s, 1H), 6.89-6.92 (m, 1H), 6.79-6.84 (m, 1H), 4.38 (q, J=7.2 Hz, 2H), 4.15 (q, J=7.2 Hz, 2H), 1.54 (t, J=7.2 Hz, 3H), 1.40 (t, J=7.2 Hz, 3H).

### Synthesis of Intermediate 49

To a solution of Intermediate 48 (10 g, 35.5 mmol) in toluene (20 mL) was added benzylamine (4.6 g, 42.6 mmol) and triethylamine (11 g, 107 mmol). The mixture was stirred at 100° C. for 4 hrs. TLC (DCM/MeOH=20/1) showed the reaction was complete. The mixture was poured into ice-water, extracted with DCM, the organic phase was washed with brine, dried over Na₂SO₄ and concentrated to give a residue. The solid was triturated with MTBE and filtered to give Intermediate 49 (14 g) as a white solid.

¹H-NMR (CDCl₃, 400 MHz, 400 MHz): δ (ppm) 11.11 (br s, 1H), 8.31 (s, 1H), 7.30-7.37 (m, 4H), 7.25-7.27 (m, 1H), 6.12 (dd, J=2.0, 10.8 Hz, 1H), 6.05 (dd, J=2.4, 12.4 Hz, 1H), 4.37-4.42 (m, 4H), 4.04-4.09 (m, 2H), 1.49 (t, J=7.2 Hz, 3H), 1.40 (t, J=7.2 Hz, 3H).

### Synthesis of Intermediate 50

To a solution of Intermediate 49 (14.0 g, 38 mmol) in EtOH (140 mL) and acetic acid (140 mL) was added Pd/C (2.8 g, 10% wt). The mixture was stirred at 30-40° C. for 4-5 hrs under H₂ at 1 atm. TLC (DCM/MeOH=20/1) showed the reaction was complete. Then the mixture was filtered, concentrated to give a crude, which was triturated with (PE/EA=15/1, 150 mL), filtered to give Intermediate 50 (9 g, 84.9%) as a white solid.

¹H NMR (DMSO-d₆, 400 MHz): δ (ppm) 8.46 (s, 1H), 6.48 (dd, J=2.0, 11.6 Hz, 1H), 6.28 (dd, J=2.4, 11.6 Hz, 1H), 4.20 (q, J=7.2 Hz, 4H), 1.25-1.31 (m, 6H).

### Synthesis of Intermediate 51

A mixture of Intermediate 50 (9.0 g, 32 mmol) in HCl (6N, 90 mL) was stirred at 100° C. for 3 hrs. The mixture was filtered to give Intermediate 51 (6.2 g, 77.5%) as a white solid.

¹H NMR (DMSO-d₆, 400 MHz): δ (ppm) 15.05 (s, 1H), 8.84 (s, 1H), 7.33 (br s, 2H), 6.72 (dd, J=2.0, 11.2 Hz, 1H), 6.45 (dd, J=2.0, 11.6 Hz, 1H), 4.38 (q, J=7.2 Hz, 2H), 1.33 (t, J=7.2 Hz, 3H).

### Synthesis of Compound I-56

To a solution of intermediate 51 (1.5 g, 6 mmol) in DMSO (40 mL) was added Phenethylamine (2.2 g, 18 mmol) and triethylamine (1.8g, 18 mmol), then the mixture was stirred at 100° C. for overnight. The mixture was poured into ice water, extracted with DCM/MeOH (10/1), the organic phase was washed with brine, dried over Na₂SO₄ and concentrated to give residue, which was purified by HPLC to give Compound I-56 (285 mg, 13.6%) as a yellow solid.

¹H NMR (DMSO-d₆, 400 MHz): δ (ppm) 15.82 (s, 1H), 8.60 (s, 1H), 7.29-7.32 (m, 6H), 7.22-7.24 (m, 1H), 6.71 (brs, 1H), 5.92-5.93 (m, 2H), 4.21-4.23 (m, 2H), 3.31-3.36 (m, 2H), 2.84-2.88 (m, 2H), 1.35 (t, J=6.8 Hz, 3H).

### Synthesis of Compound I-57

To a solution of Intermediate 51 (1.5 g, 6 mmol) in DMSO (40 mL) was added 3-phenylpropan-1-amine (2.4 g, 18 mmol) and triethylamine (1.8 g, 18 mmol), then the mixture was stirred at 100° C. for overnight. The mixture was poured into ice water, extracted with DCM/MeOH (10/1), the organic phase was washed with brine, dried over Na₂SO₄ and concentrated. The crude material was purified by HPLC to give Compound I-57 (282 mg, 12.9%) as a yellow solid.

¹H NMR (DMSO-d₆, 400 MHz): δ (ppm) 15.83 (s, 1H), 8.60 (s, 1H), 7.19-7.32 (m, 7H), 6.67 (brs, 1H), 5.85-5.89 (m, 2H), 4.19-4.21 (m, 2H), 3.09-3.10 (m, 2H), 1.84-1.88 (m, 2H), 1.33 (t, J=7.2 Hz, 3H).

### Synthesis of Compound I-58

To a solution of Intermediate 51 (1.5 g, 6 mmol) in DMSO (40 mL) was added cyclohexylamine (1.79g, 18 mmol) and triethylamine (1.82g, 18 mmol), then the mixture was stirred at 100° C. for overnight. The mixture was poured into ice water, extracted with DCM/MeOH (10/1), the organic phase was washed with brine, dried over Na₂SO₄ and concentrated to give residue, which was purified by HPLC to give Compound I-58 (230 mg, 11.7%) as a brown solid.

¹H NMR (DMSO-d₆, 400 MHz): δ (ppm) 15.85 (s, 1H), 8.57 (s, 1H), 7.38-7.39 (br s, 1H), 6.46-6.48 (m, 1H), 5.93 (s, 1H), 5.87 (s, 111), 4.19 (q, J=7.2 Hz, 2H), 3.26-3.28 (m, 111), 1.91-1.93 (m, 2H), 1.72-1.75 (m, 2H), 1.59-1.62 (m, 1H), 1.30-1.39 (m, 5H), 1.14-1.23 (m, 3H).

### Synthesis of Compound I-59

To a solution of Intermediate 51 (100 mg, 0.4 mmol) in DMSO (3 mL) was added tranylcypromine trans relative (160 mg, 1.2 mmol) and triethylamine (121.2 mg, 1.2 mmol), then the mixture was stirred at 80° C. for overnight. The mixture was poured into ice water, extracted with DCM/MeOH(10/1), the organic phase was washed with brine, dried over Na₂SO₄ and concentrated to give residue, which was purified by HPLC to give Compound I-59 trans relative (21 mg, 14.5%) as a white solid.

¹H NMR (DMSO-d₆, 400 MHz): δ (ppm) 15.75 (s, 1H), 8.57 (s, 1H), 7.42 (br s, 2H), 7.29-7.33 (m, 2H), 7.18-7.23 (m, 4H), 5.92 (s, 1H), 5.86 (s, 1H), 3.99-4.00 (m, 2H), 2.62 (s, 1H), 1.93-1.94 (m, 1H), 1.28-1.32 (m, 2H), 1.05 (t, 3H).

### Synthesis of Compound I-60

To a solution of Compound I-6 (0.5 g, 1.9 mmol) in DMSO (15 mL) was added trans 3-phenyl-cyclobutylamine (0.4 g, 2.8 mmol) and triethylamine (0.6 g, 5.6 mmol), then the mixture was stirred at 100° C. for 16 hrs. The mixture was poured into ice water, filtered. The filtered cake was added into EtOH and stirred at 40° C. for 30 min, then filtered to give Compound I-60 (105 mg, 14.0%) as a white solid.

1H NMR (DMSO-d₆, 400 MHz): δ (ppm) 8.69 (s, 1H), 7.32-7.35 (m, 4H), 7.19-7.23 (m, 3H), 7.01 (d, J=4.4 Hz, 1H), 5.81 (d, J=7.2 Hz, 1H), 4.34 (q, J=6.8 Hz, 2H), 4.21-4.26 (m, 1H), 3.67-3.71 (m, 1H), 2.53-2.57 (m, 4H), 1.33 (t, J=6.8 Hz, 3H); MS: 396.0 [M+1].

### Synthesis of Compound I-61

To a solution of Compound I-6 (0.5 g, 1.9 mmol) in DMSO (16 mL) was added Cis-3-phenyl-cyclobutylamine (0.4 g, 2.8 mmol) and triethylamine (0.6 g, 5.6 mmol), then the mixture was stirred at 100° C. for 16 hrs. The mixture was poured into ice water, filtered, the filtered cake was washed with EtOH, then added into MeOH/PE=1/15 and stirred at room temperature for 40 min, filtered to give Compound I-61 (114 mg, 15.2%) as a yellow solid.

¹H NMR (DMSO-d₆, 400 MHz): δ (ppm) 8.70 (s, 1H), 7.29-7.32 (m, 4H), 7.17-7.21 (m, 3H), 6.89 (d, J=6.8 Hz, 1H), 6.03 (d, J=6.8 Hz, 1H), 4.38 (q, J=7.2 Hz, 2H), 4.11-4.17 (m, 1H), 3.20-3.24 (m, 1H), 2.78-2.84 (m, 2H), 2.13-2.21 (m, 2H), 1.36 (t, J=7.2 Hz, 3H); MS: 396.0 [M+1].

### Example 13: Synthesis of Compound I-62

Synthetic Route to Intermediate 57

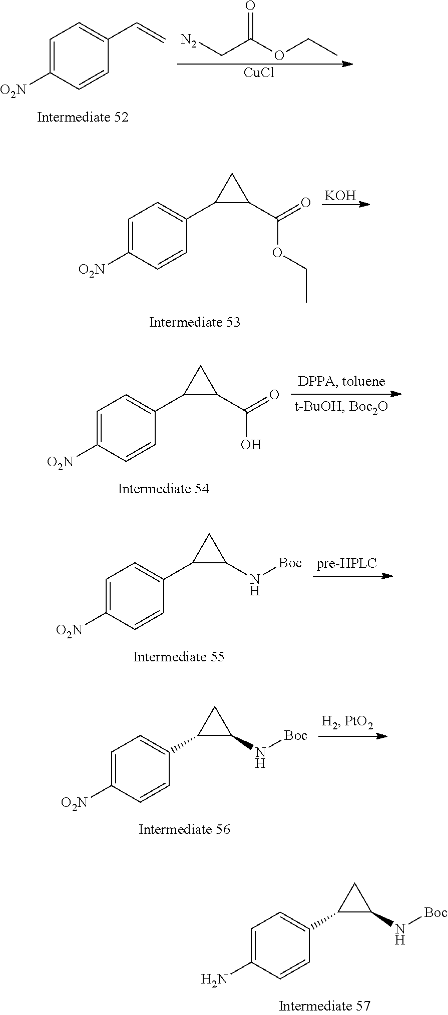

### Synthesis of Intermediate 53

To a solution of Intermediate 52 (5 g, 33.6 mol) in CDCl₃ (50 mL) was added ethyl diazoacetate (6 mL, 57.3 mol), CuCl (0.2 g, 3.4 mmol) was added, the mixture was stirred at 50° C., after that the mixture was heated to reflux for 16 hrs, the mixture was concentrated to give crude material, which was purified by silica gel chromatography to give Intermediate 53 (2 g, 24%) as a yellow oil.

¹H NMR (CDCl₃, 400 MHz): δ (ppm) 8.14 (d, J=8.4 Hz, 2H), 7.24 (d, J=8.8 Hz, 2H), 4.19 (q, J=7.2 Hz, 2H), 2.57-2.63 (m, 1H), 1.98-2.03 (m, 1H), 1.71-1.75 (m, 1H), 1.36-1.41 (m, 1H), 1.29 (t, J=7.2 Hz, 3H).

### Synthesis of Intermediate 54

To a solution of Intermediate 53 (16 g, crude, 58 mmol) in EtOH (116 mL) was added KOH (13.2 g, 231 mmol) in water (116 mL), the mixture was stirred at RT for 16 hrs. The mixture was poured into water, adjusted pH=3-4 with HCl (2N), filtered to give Intermediate 53 (5 g, 38%) as a yellow solid.

¹H NMR (DMSO-d₆, 400 MHz): δ (ppm) 12.32 (bs, 1H), 8.12 (d, J=8.8 Hz, 2H), 7.44 (d, J=8.8 Hz, 2H), 2.55-2.60 (m, 1H), 1.96-2.01 (m, 1H), 1.44-1.53 (m, 2H).

### Synthesis of Intermediate 55

To a solution of Intermediate 54 (2 g, 9.6 mmol) in toluene (40 mL) was added Et₃N (1.2 g, 11.6 mmol) and diphenylphosphoryl azide (DPPA) (3.2 g, 11.6 mmol) at RT under N₂, the mixture was stirred at RT for 0.5 hr. t-BuOH (7.2 g, 96 mmol) was added, the mixture was stirred at 80° C. for 16 hrs, cooled, then Boc₂O (3.2 g, 14.4 mmol) was added, The mixture was stirred at 80° C. for another 2 hrs, poured into water, extracted with Ethyl acetate, concentrated and purified by silica gel chromatography to give Intermediate 55 (1.6 g, crude) as a yellow solid.

### Synthesis of Intermediate 56

A solution of Intermediate 55 (4 g, crude) in MeCN (120 mL) was separated by prep HPLC to give Intermediate 56 trans relative (1.3 g, 65%) as off white solid.

¹H NMR (CDCl₃, 400 MHz): δ (ppm) 8.12 (d, J=8.8 Hz, 2H), 7.25 (d, J=8.0 Hz, 2H), 4.88 (brs, 1H), 2.70-2.80 (m, 1H), 2.12-2.16 (m, 1H), 1.44 (s, 9H), 1.25-1.33 (m, 2H).

### Synthesis of Intermediate 57

To a solution of Intermediate 56 trans relative (1.3 g, 4.7 mmol) in MeOH (30 mL) was added PtO₂ (1.0 g). The mixture was stirred at 40° C. for 4-5 hrs under H₂ at 1 atm. TLC (EA/PE=113) showed the reaction was complete. The mixture was filtered, concentrated to give crude Intermediate 57 trans relative (1.0 g, 80%) as a white solid.

¹H NMR (CDCl₃, 400 MHz): δ (ppm) 6.94-6.99 (m, 2H), 6.55-6.60 (m, 2H), 4.70-4.90 (brs, 1H), 3.40-3.80 (brs, 2H), 2.52-2.62 (m, 1H), 1.85-1.93 (m, 1H), 1.45 (s, 9H), 1.04-1.07 (m, 2H).

### Example 14: Synthetic Route to Compound I-62

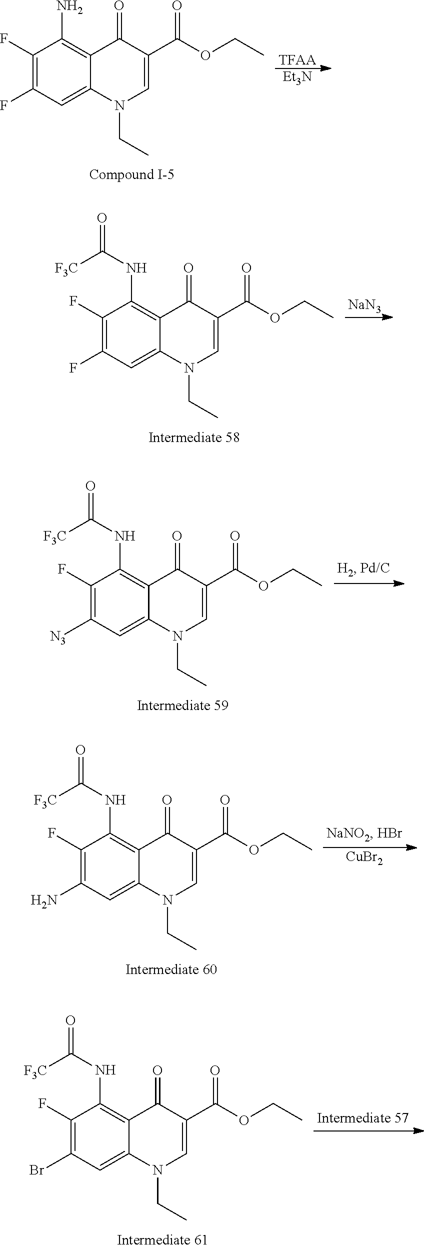

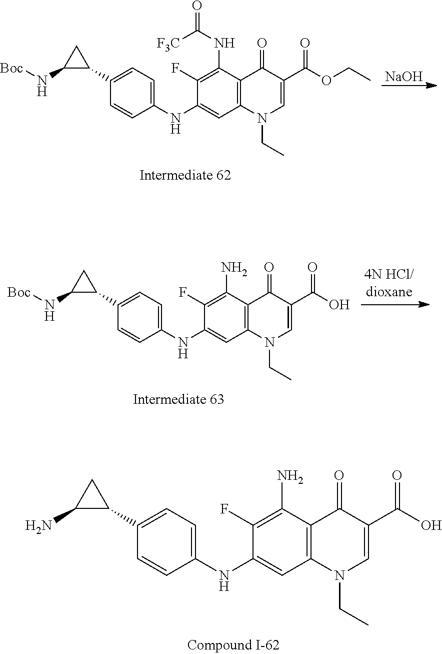

### Synthesis of Intermediate 58

To a solution of Compound I-5 (3.0 g, 10.1 mmol) in THF (60 mL) was added Diisopropylethylamine (3.7 g, 28.3 mmol), the mixture was cooled to −10˜−20° C., Trifluoroacetic anhydride (4.2 g, 20.2 mmol) in THF (10 mL) was added. The mixture was stirred for another 2 hrs, poured into water, extracted with EtOAc, the organic phase was washed with brine, dried over Na₂SO₄ and concentrated in vacuum, then purified by silica gel chromatography to give Intermediate 58 (1.8 g, 45.4%) as a yellow solid.

¹H NMR (CDCl₃, 400 MHz): δ (ppm) 13.51 (s, 1H), 8.50 (s, 1H), 7.16-7.21 (m, 1H), 4.38-4.44 (q, 2H), 4.22-4.28 (q, 2H), 1.58 (t, J=7.2 Hz, 3H), 1.41 (t, J=7.2 Hz, 3H).

### Synthesis of Intermediate 59

To a solution of Intermediate 58 (1.8 g, 4.6 mmol) in DMF (60 mL) was added NaN₃ (0.9 g, 13.8 mmol), the mixture was stirred at 65° C. for 6 hrs. The mixture was poured into water, extracted with EtOAc, the organic phase was washed with brine, dried over Na₂SO₄ and concentrated in vacuum to give a crude of Intermediate 59 (1.6 g, crude) as a yellow solid.

### Synthesis of Intermediate 60

To a solution of Intermediate 59 (1.6 g, crude, 4.1 mmol) in EtOH/THF (30 mL/30 mL) was added Pd/C (1.5 g, 10% wt), the mixture was stirred at 40° C., 50 psi under H₂ for 1 hour, filtered, and the filtered cake was washed with MeOH/DCM/THF=1/1/1. The filtrate was concentrated to give crude material, MTBE/PE=1/1 was added. After stirring for 30 min at RT, Intermediate 60 (1.1 g, 73%) as a yellow solid.

¹H NMR (DMSO-d₆, 400 MHz): δ (ppm) 13.67 (s, 1H), 8.58 (s, 1H), 6.86 (d, J=7.2 Hz, 1H), 6.57 (s, 2H), 4.18-4.28 (m, 4H), 1.37 (t, J=7.2 Hz, 3H), 1.25 (t, J=7.2 Hz, 3H).

### Synthesis of Intermediate 61

To a solution of Intermediate 60 (1.1 g, 2.8 mmol) in HBr (110 mL, 20%) was cooled to 0° C., NaNO₂ (1.0 g, 14.1 mmol) was added, the mixture was stirred at 0° C. for 1 hrs, CuBr₂ (0.8 g, 5.7 mmol), the mixture was added, the mixture was stirred at RT for 1 hour, the mixture was poured into ice-water, extracted with EtOAc, and concentrated to give a crude, which was added into MTBE/PE=1/1 and stirred for 30 min, filtered to give Intermediate 61 (0.45 g, 34.6%) as a yellow solid.

¹H NMR (CDCl₃, 400 MHz): δ (ppm) 13.34 (s, 1H), 8.49 (s, 1H), 7.60 (d, J=4.8 Hz, 1H), 4.36-4.42 (m, 2H), 4.25-4.30 (m, 2H), 1.60 (t, J=7.2 Hz, 3H), 1.40 (t, J=7.2 Hz, 3H).

### Synthesis of Intermediate 62

To a solution of Intermediate 61 (0.45 g, 1.0 mmol) in dioxane (40 mL) was added Intermediate 57 trans relative (0.37 g, 1.5 mmol), Pd(Ac)₂ (0.07 g, 0.3 mmol), Xantphos (0.30 g, 0.45 mmol) and Cs₂CO₃ (0.5 g, 1.5 mmol) under N₂, the mixture was stirred at 100° C. for 2 hrs. The mixture was concentrated in vacuum, purified by pre-TLC to give Intermediate 62 trans relative (200 mg, 32.2%) as pink solid.

¹H NMR (CDCl₃, 400 MHz): δ (ppm) 13.71 (s, 1H), 8.36 (s, 1H), 7.16-7.26 (m, 4H), 6.81 (d, J=6.4 Hz, 1H), 6.54 (d, J=3.2 Hz, 1H), 4.89 (bs, 1H), 4.35-4.40 (m, 2H), 4.02-4.08 (m, 2H), 2.75 (m, 1H), 2.04-2.10 (m, 1H), 1.37-1.45 (m, 15H), 1.18-1.19 (m, 2H).

### Synthesis of Intermediate 63

A mixture of Intermediate 62 trans relative (200 mg, 0.3 mmol) in THE (10 mL) was added NaOH (2 N, 10 mL), the mixture was stirred at 28° C. for 9 hrs. The mixture was adjusted pH=6˜7 with HCl (1N), extracted with EtOAc, the organic phase was washed with brine, dried over Na₂SO₄ and concentrated in vacuum to give a crude, which was purified by pre-HPLC to give Intermediate 63 trans relative (30 mg, 18.8%) as a yellow oil.

¹H NMR (CD₃OD+CDCl₃, 400 MHz): δ (ppm) 8.50 (s, 1H), 7.16-7.22 (m, 4H), 6.33 (d, J=6.4 Hz, 1H), 4.06-4.12 (m, 2H), 2.69 (m, 1H), 2.04-2.05 (m, 1H), 1.41-1.46 (m, 12H), 1.14-1.20 (m, 2H).

### Synthesis of Compound I-62

A mixture of Intermediate 63 trans relative (30 mg, 0.06 mmol) in HCL/MTBE (4 N, 5 mL) and DCM (2 mL) was stirred at room temperature for 3 hrs, filtered to give Compound I-62 trans relative (15 mg, 62.5%) as a brown solid.

¹H NMR (CD₃OD, 400 MHz): δ (ppm) 8.62 (s, 1H), 7.13-7.19 (m, 4H), 4.09 (bs, 2H), 2.7-2.8 (m, 1H), 2.3-2.4 (m, 1H), 1.24-1.30 (m, 5H).

### Example 15: Synthesis of Compound I-63

Synthetic route:

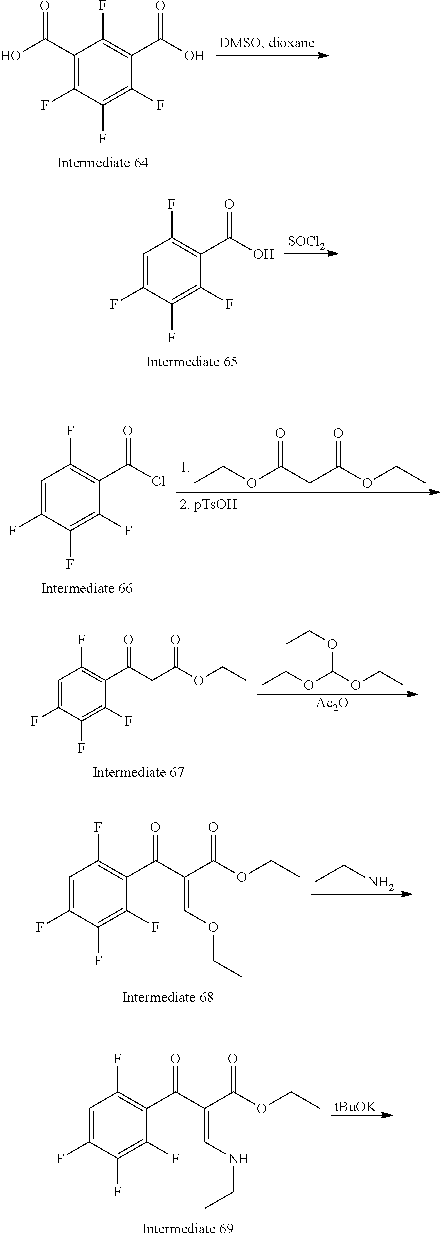

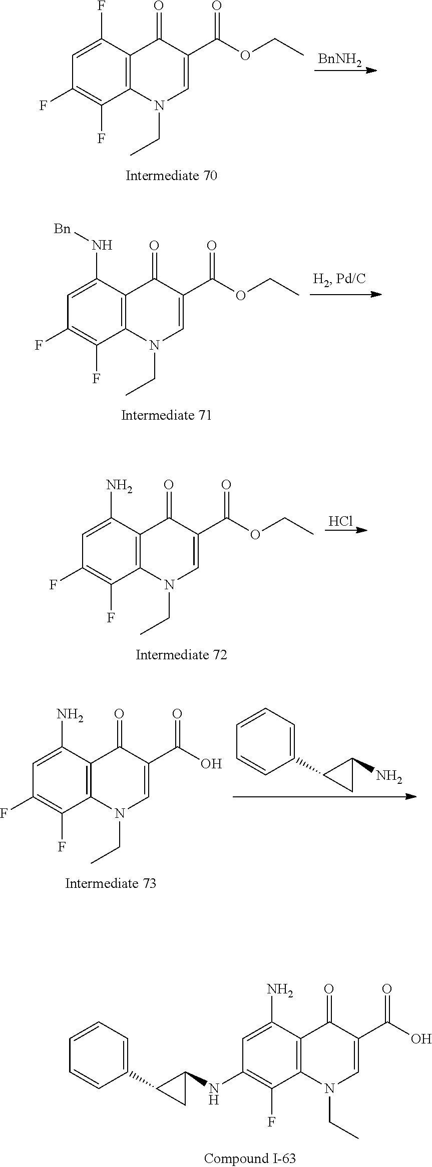

### Synthesis of Intermediate 65

To a solution of Intermediate 64 (15 g, 63.0 mmol) in DMSO (30 mL) was added dioxane (20 mL), the mixture was heated to 135° C. for 4 hrs, then 150° C. for 30 min. The mixture was added ice water, extracted with toluene and ether, the organic phase was concentrated in vacuum to give a crude, EA/hexane=1/2 was added, the mixture was stirred at room temperature for 30 min, and filtered to give Intermediate 65 (7 g, 58%) as a yellow solid.

¹H NMR (CDCl₃, 400 MHz): δ (ppm) 14.25 (br s, 1H), 7.61-7.68 (m, 1H).

### Synthesis of Intermediate 66

To a solution of Intermediate 65 (14 g, 72.2 mmol) in SOCl₂ (11.2 g, 93.8 mmol) was added DMF (0.05 g, cat), the mixture was stirred at 90° C. for 2 hrs, 120° C. for 1 hour, and 140° C. for 2 hrs. The mixture was concentrated in vacuum to give Intermediate 66 (14 g, crude) as a yellow oil.

### Synthesis of Intermediate 67

A mixture of diethyl malonate (22.1 g, 138.4 mmol) NaOEt (9 g, 131.8 mmol) in toluene (100 ml) was stirred at RT for 1 hour, Intermediate 66 (14 g, crude, 65.9 mmol) in toluene (20 ml) was added at 0˜5° C., the mixture solution was stirred at RT for 1 hour. The mixture was poured into NaOH (2 N), the aqueous layer was combined and extracted with hexane, then adjusted pH=3˜4 with HCl (2N), extracted with DCM, concentrated in vacuum to give a residue, which was added into water (80 mL) and p-TsOH (63.5 g 369.04 mmol), the mixture was heated to reflux for 3 hrs, poured into NaHCO₃ (aq), extracted with DCM, concentrated in vacuum to give a crude, which was purified by silica gel chromatography to give crude Intermediate 67 (8.9 g, 52%) as a yellow oil.

¹H NMR (CDCl₃, 400 MHz): δ (ppm) 12.37 (s, 0.38H), 6.83-6.90 (m, 1H), 5.40 (s, 0.38H), 4.11-4.31 (m, 2.8H), 3.89 (s, 1.2H), 1.25-1.41 (m, 3H).

### Synthesis of Intermediate 68

A mixture of Intermediate 67 (8.9 g, 33.7 mmol), triethoxymethane (8.5 g, 57.3 mmol) in acetic anhydride (9.6 g, 94.3 mmol) was heated to 140° C. for 2 hrs, the mixture was concentrated, toluene was added, concentrated in vacuum to give a crude of Intermediate 68 (10 g, crude) as a yellow oil.

### Synthesis of Intermediate 69

To a solution of Intermediate 68 (10.0 g, crude, 31.2 mmol) in EtOH (50 mL) was added Ethylamine (1.7 g, 37.5 mmol), then the mixture was stirred at 28° C. for 16 hrs. The mixture was concentrated in vacuum to give a residue, EtOH and hexane were added, and the filtered to give crude Intermediate 69 (8.5 g, 85%) as a yellow solid.

### Synthesis of Intermediate 70

To a solution of Intermediate 69 (8.5 g, 26.6 mmol) in THF/dioxane (30 mL/30 mL) was added t-BuOK (3.1 g, 28.0 mmol) at 0-5° C. under N₂, the mixture was stirred at 0-5° C. for 1 hour. The mixture was poured into ice water, filtered to give Intermediate 70 (7.2 g, 90%) as a yellow solid.

¹H NMR (DMSO-d₆, 400 MHz): δ (ppm) 8.54 (s, 1H), 7.56-7.58 (m, 1H), 4.38-4.41 (m, 2H), 4.19-4.25 (m, 2H), 1.35-1.41 (m, 3H), 1.27 (t, J=6.8 Hz, 3H).

### Synthesis of Intermediate 71

To a solution of Intermediate 70 (7.2 g, 24.0 mmol) in toluene (140 mL) was added phenylmethanamine (5.1 g, 48 mmol) and triethylamine (7.3 g, 72.0 mmol). The mixture was stirred at 100° C. for 6 hrs. The mixture was poured into ice-water, extracted with DCM, the organic phase was washed with brine, dried over Na₂SO₄ and concentrated to give a residue, which was dissolved into MTBE, filtered to give Intermediate 71 (6.1 g, 65.6%) as a yellow solid.

¹H NMR (DMSO-d₆, 400 MHz): δ (ppm) 10.79 (s, 1H), 8.45 (s, 1H), 7.30-7.34 (m, 5H), 7.26-7.29 (m, 1H), 6.45 (dd, J=6.4 Hz, J=13.6 Hz, 1H), 4.41-4.44 (m, 2H), 4.32-4.36 (m, 2H), 4.18-4.23 (m, 2H), 1.34 (t, J=6.4 Hz, 3H), 1.27 (t, J=7.2 Hz, 3H).

### Synthesis of Intermediate 72

To a solution of Intermediate 71 (6.1 g, 15.8 mmol) in EtOH (60 mL) and acetic acid (60 mL) was added Pd/C (4.0 g, 10% wt). The mixture was stirred at 30-40° C. for 6 hrs under H₂ at 1 atm. Then the mixture was filtered, concentrated to give a residue, which purified by silica gel chromatography to give Intermediate 72 (4.2 g, 89.0%) as a yellow solid.

¹H NMR (DMSO-d₆, 400 MHz): δ (ppm) 8.59 (s, 1H), 7.93 (br s, 1H), 6.43-6.48 (dd, J=6.4 Hz, J=12.8 Hz, 1H), 4.28-4.39 (m, 2H), 4.16-4.21 (m, 2H), 1.33 (t, J=6.8 Hz, 3H), 1.24 (t, J=6.8 Hz, 3H).

### Synthesis of Intermediate 73

A mixture of Intermediate 72 (4.2 g, 14.2 mmol) in HCl (6N, 40 mL) was stirred at 100° C. for 6 hrs, cooled. Then the mixture was filtered to give Intermediate 73 (2.6 g, 68.4%) as a brown solid.

¹H NMR (DMSO-d₆, 400 MHz): δ (ppm) 14.74 (s, 1H), 8.77 (s, 1H), 7.86 (brs, 2H), 6.63 (dd, J=6.4 Hz, J=12.8 Hz 1H), 4.44-4.48 (m, 2H), 1.38 (t, J=6.8 Hz, 3H).

### Synthesis of Compound I-63

To a solution of Intermediate 73 (1.3 g, 4.9 mmol) in DMSO (28 mL) was added triethylamine (1.5 g, 14.6 mmol), then relative trans-2-phenylcyclopropanamine (1.9 g, 14.6 mmol) was added. The mixture was stirred at 100° C. for 16 hrs. The mixture was poured into ice-water, filtered, the filtered cake was purified by pre-TLC to give Compound I-63 trans relative (223 mg, 12.3%) as a yellow solid.

¹H NMR (DMSO-d₆, 400 MHz): δ (ppm) 15.38 (s, 1H), 8.54 (s, 1H), 7.47 (s, 2H), 7.29-7.33 (m, 2H), 7.17-7.22 (m, 3H), 7.07 (s, 1H), 6.09 (d, J=6.8 Hz, 1H), 4.37-4.40 (m, 2H), 2.65-2.67 (m, 1H), 2.08-2.11 (m, 1H), 1.27-1.37 (m, 5H); MS: 382.0 [M+1].

### Synthesis of Compound I-64

To a solution of Intermediate 73 (1.3 g, 4.9 mmol) in DMSO (28 mL) was added triethylamine (1.5 g, 14.6 mmol), then cyclohexanamine (1.5 g, 14.6 mmol) was added. The mixture was stirred at 100° C. for 16 hrs, cooled. Then the mixture was poured into ice-water, filtered, the filtered cake was purified by pre-TLC to give Compound I-64 (102 mg, 6.2%) as a yellow solid.

¹H NMR (DMSO-d₆, 400 MHz): δ (ppm) 15.46 (s, 1H), 8.50 (s, 1H), 7.38 (s, 2H), 6.13-6.15 (m, 1H), 6.07 (d, J=6.8 Hz, 1H), 4.35-4.38 (m, 2H), 3.21 (s, 1H), 1.91-1.92 (m, 2H), 1.73-1.74 (m, 2H), 1.61-1.64 (m, 1H), 1.23-1.35 (m, 7H), 1.12-1.15 (m, 1H); MS: 348.1 [M+1].

### Synthesis of Compound I-65

To a solution of Intermediate 73 (0.6 g, 2.2 mmol) in DMSO (18 mL) was added triethylamine (0.7 g, 6.6 mmol), then 2-phenylethanamine (0.8 g, 6.6 mmol) was added. The mixture was stirred at 100° C. for 16 hrs, cooled. Then the mixture was poured into ice-water, filtered, the filtered cake was purified by pre-TLC to give Compound I-65 (111 mg, 13.6%) as a yellow solid.

¹H NMR (DMSO-d₆, 400 MHz): δ (ppm) 15.45 (s, 1H), 8.52 (s, 1H), 7.34-7.45 (m, 2H), 7.29-7.32 (m, 4H), 7.21-7.25 (m, 1H), 6.61-6.62 (m, 1H), 6.13 (d, J=7.2 Hz, 1H), 4.36-4.39 (m, 2H), 3.32-3.36 (m, 2H), 2.86-2.89 (m, 2H), 1.34 (t, J=6.8 Hz, 3H).

### Example 16: Synthesis of Compound I-66, Compound I-67, Compound I-68

Synthetic route:

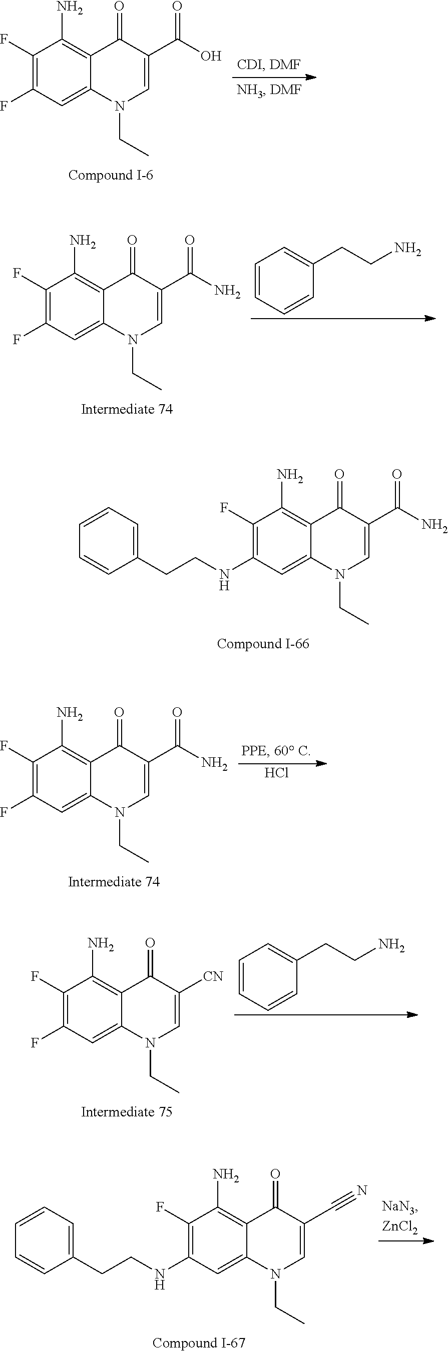

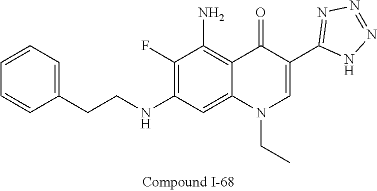 [SMILES: CCn1cc(-c2nnn[nH]2)c(=O)c2c(N)c(F)c(NCCc3ccccc3)cc21]

### Synthesis of Intermediate 74

To a solution of Compound I-6 (6.0 g, 22.4 mmol) in DMF (66 mL) was added CDI (5.4 g, 33.6 mmol). The mixture was warm up to 100° C. for 4 hrs. Cooled and poured into water and filtered, the filtered cake was dried in vacuum to give off-white solid (5.0 g crude).

The off-white solid was dissolved into DMF (100 mL) at 25-35° C., then anhydrous NH₃ was bubbled though this solution at 25-35° C. for 30 min, then the mixture was stirred for another 2 hrs, poured into water, filtered to give Intermediate 74 (3.0 g, 50%) as a white solid

¹H NMR (DMSO-d₆, 400 MHz): δ (ppm) 9.03 (brs, 1H), 8.71 (s, 1H), 7.43 (brs, 1H), 6.88 (dd, J=6.4, 12.8 Hz, 1H), 4.29 (q, J=7.2 Hz, 2H), 1.31 (t, J=7.2 Hz, 3H).

### Synthesis of Compound I-66

To a solution of Intermediate 74 (0.5 g, 1.9 mmol) in DMSO (15 mL) was added 2-phenylethanamine (0.7 g, 5.6 mmol) and triethylamine (0.6 g, 5.6 mmol), then the mixture was stirred at 90° C. for 16 hrs. The mixture was poured into ice water, filtered, the filtered cake purified by pre-HPLC to give Compound I-66 (38 mg, 5.4%) as a white solid.

¹H NMR (DMSO-d₆, 400 MHz): δ (ppm) 9.29 (d, J=4.8 Hz, 1H), 8.53 (s, 1H), 7.19-7.33 (m, 7H), 6.29-6.31 (m, 1H), 5.93 (d, J=6.8 Hz, 1H), 4.27 (q, J=6.8 Hz, 2H), 3.43-3.48 (m, 2H), 2.88-2.92 (m, 2H), 1.33 (t, J=6.8 Hz, 3H); MS: 369.2 [M+1].

### Synthesis of Intermediate 75

To a solution of Intermediate 74 (3.0 g, 11.1 mol) and PPE (30 g) was warm up to 65° C. for 4 hrs, the mixture was cooled and HCl (1 N, 100 mL) was added, the mixture was stirred for 1 hour, filtered, the filtered cake was purified by silica gel chromatography (DCM/MeOH=2001˜50/1) to give Intermediate 75 (1.0 g, 35.7%) as a white solid.

¹H NMR (DMSO-d₆, 400 MHz): δ (ppm) 8.70 (s, 1H), 7.91 (br s, 1H), 6.88 (dd, J=6.4, 13.2 Hz, 1H), 4.20 (q, J=7.2 Hz, 2H), 1.31 (t, J=7.2 Hz, 3H).

### Synthesis of Compound I-67

To a solution of Intermediate 75 (1.0 g, 4.0 mmol) in DMSO (20 mL) was added 2-phenylethanamine (1.4 g, 12 mmol) and triethylamine (1.2 g, 12 mmol), then the mixture was stirred at 100° C. for 16 hrs. The mixture was poured into ice water, filtered. The filtered cake was purified by pre-TLC to give Compound I-67 (300 mg, 21.4%) as a white solid.

1H NMR (DMSO-d₆, 400 MHz): δ (ppm) 8.48 (s, 1H), 7.19-7.33 (m, 7H), 6.39-6.41 (m, 1H), 5.91-5.93 (d, J=6.8 Hz, 1H), 4.16 (q, J=6.8 Hz, 2H), 3.43-3.48 (m, 2H), 2.87-2.91 (m, 2H), 1.33 (t, J=6.8 Hz, 3H); MS: 351.0 [M+1].

### Synthesis of Compound I-68

To a solution of Compound I-67 (0.3 g, 0.9 mmol) in iPrOH/H₂O (9 mL/3 mL) was added NaN₃ (0.3 g, 4.3 mmol) and ZnCl₂ (0.6 g, 4.3 mmol), then the mixture was stirred at 90° C. for 16 hrs. The mixture was poured into ice water, filtered. The filtered cake was dissolved with DMSO at 50° C., filtered. The filtered cake was added into MeOH, reflux for 2 hrs, filtered, washed with water to give Compound I-68 (192 mg, 57%) as a yellow solid.

¹H NMR (DMSO-d₆, 400 MHz): δ (ppm) 9.03 (s, 1H), 7.64 (br s, 2H), 7.30-7.33 (m, 4H), 7.21-7.23 (m, 1H), 6.46-6.48 (m, 1H), 6.03 (d, J=6.4 Hz, 1H), 4.43-4.45 (m, 2H), 3.50-3.52 (m, 2H), 2.93-2.96 (m, 2H), 1.43 (t, J=6.8 Hz, 3H); MS: 395.0 [M+1].

### Synthesis of Compound I-69

To a solution of Compound I-6 (0.5 g, 1.9 mmol) in DMSO (16 mL) was added 1-phenyl-3-Azetidinamine (0.8 g, 5.6 mmol) and triethylamine (0.6 g, 5.6 mmol), then the mixture was stirred at 100° C. for 16 hrs. The mixture was poured into ice water, filtered. The filtered cake was purified by prep-HPLC to give Compound I-69 (46 mg, 6.1%) as a white solid.

¹H NMR (DMSO-d₆, 400 MHz): δ (ppm) 15.46 (s, 1H), 8.70 (s, 1H), 7.16-7.20 (m, 5H), 6.68-6.71 (m, 1H), 6.48 (d, J=7.6 Hz, 2H), 6.01 (d, J=4.2 Hz, 1H), 4.66-4.68 (m, 1H), 4.38-4.40 (m, 2H), 4.27-4.29 (m, 2H), 3.75-3.77 (m, 2H), 1.35-1.37 (m, 3H); MS: 397.0 [M+1].

### Example 17: Synthesis of Compound I-70

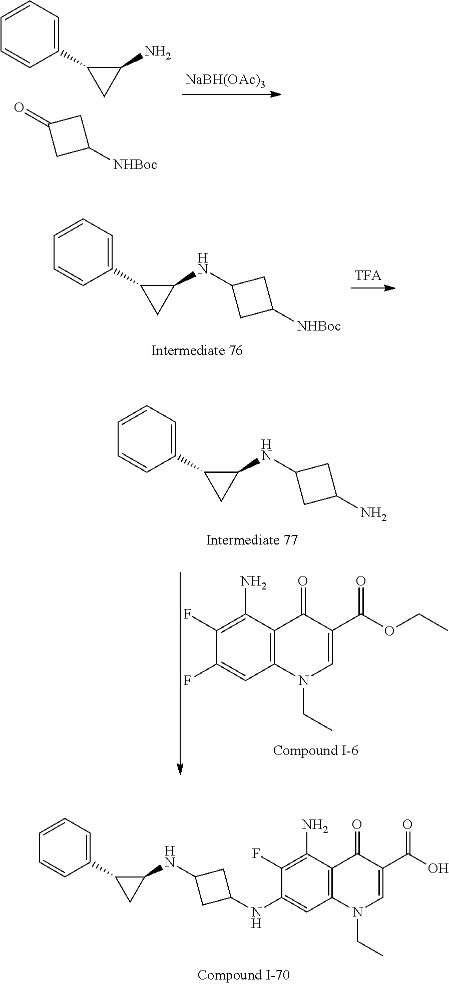

### Synthesis of Intermediate 76

To a solution of Tranylcypromine HCl trans relative (2.0 g, 11.8 mmol) in DCE (60 mL) was added tert-butyl 3-oxocyclobutylcarbamate (2.6 g, 14.2 mmol) under N₂ at 0° C., AcOH (0.7 g, 11.8 mmol) was added, 1 hour later, NaBH(OAc)₃ (4.5 g, 21.2 mmol) was added slowly. The mixture was stirred at 0-10° C. for 2 hrs, then room temperature for 16 hrs, poured into ice water, extracted with EtOAc. The organic phase was concentrated in vacuum, purified by silica gel of chromatography to give Intermediate 76 cyclopropyl trans relative (4.0 g, crude) as a yellow oil.

¹H NMR (CDCl₃, 400 MHz): δ (ppm) 7.22-7.26 (m, 2H), 7.12-7.16 (m, 1H), 7.00-7.02 (m, 2H), 4.71-4.73 (m, 1H), 4.14-4.16 (m, 1H), 3.52-3.55 (m, 1H), 2.11-2.28 (m, 4H), 1.87-1.91 (m, 1H), 1.70-1.73 (m, 1H), 1.43 (s, 9H), 0.94-1.07 (m, 2H).

### Synthesis of Intermediate 77

To a solution of Intermediate 76 cyclopropyl trans relative (4.0 g, crude) in DCM (40 mL) was added TFA (12 mL), the mixture was stirred at room temperature for 4 hrs. The mixture was concentrated, ice water was added, adjusted pH=8˜9 with Na₂CO₃ (aq), extracted with DCM/MeOH=10/1. The organic phase was dried over Na₂SO₄ and concentrated in vacuum to give Intermediate 77 cyclopropyl trans relative (2.4 g crude, 90%) as a yellow oil.

### Synthesis of Compound I-70

To a solution of Intermediate 77 cyclopropyl trans relative (1.2 g, crude) in DMSO (25 mL) was added Et₃N (0.6 g, 5.9 mmol), then Compound I-6 (0.5 g, 2.0 mmol) was added. The mixture was stirred at 75° C. for 16 hrs, cooled. Then the mixture was poured into ice-water, filtered, the crude was purified by prep-TLC to give Compound I-70 cyclopropyl trans relative (140 mg, 12.4%) as a yellow solid.

¹H NMR (DMSO-d₆, 400 MHz): δ (ppm) 15.46 (s, 1H), 9.43 (brs, 2H), 8.70 (s, 1H), 7.17-7.32 (m, 7H). 6.91-7.01 (m, 1H), 5.76-5.90 (m, 1H), 4.29-4.36 (m, 3H), 4.00-4.04 (m, 1H), 2.63-2.79 (m, 4H), 2.28-2.32 (m, 1H), 1.26-1.47 (m, 5H); MS: 451.1 [M+1].

### Example 18: Synthesis of Compound I-81 and Compound I-82

Synthetic Route

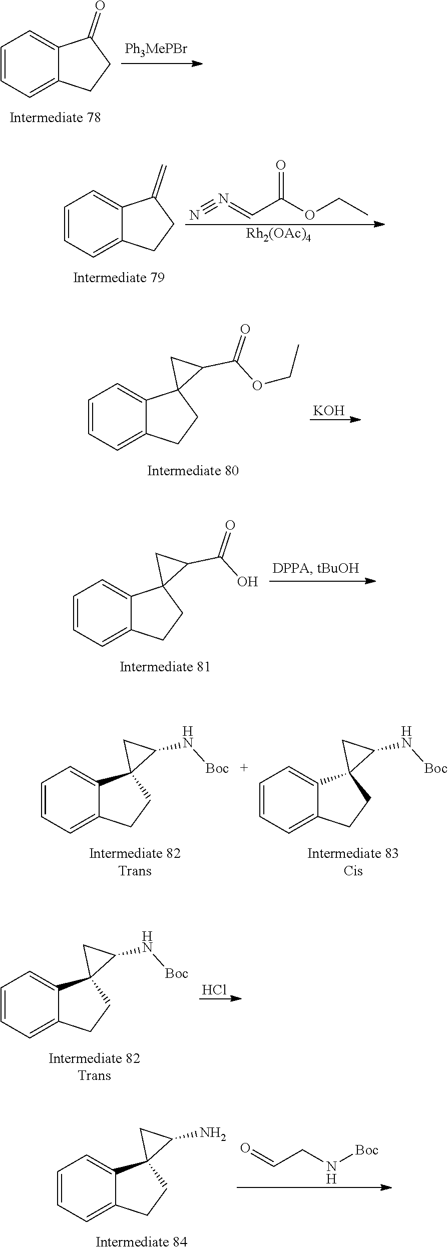

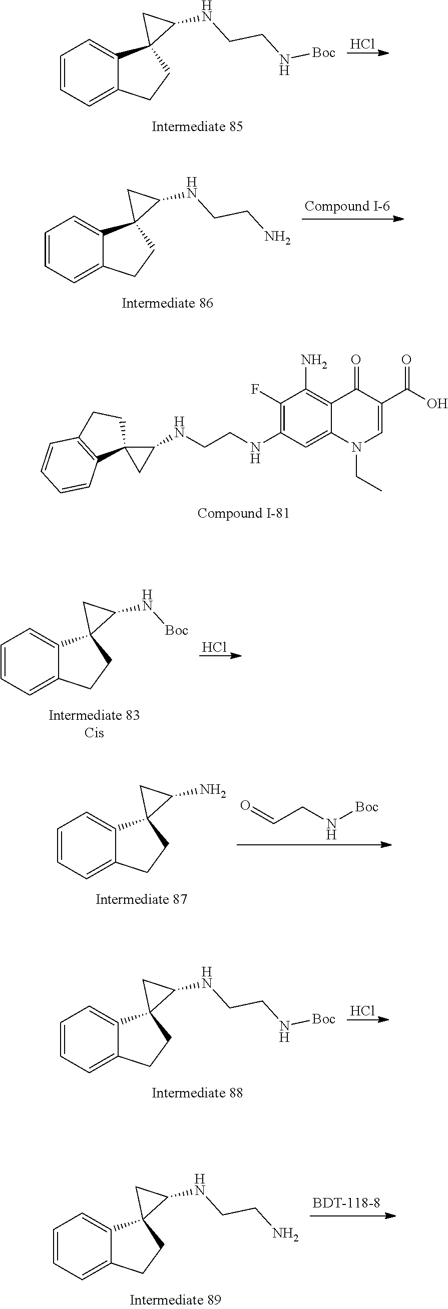

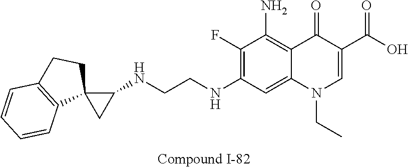 [SMILES: CCn1cc(C(=O)O)c(=O)c2c(N)c(F)c(NCCN[C@@H]3C[C@@]34CCc3ccccc34)cc21]

A mixture of Ph₃MePBr (123 g, 0.345 mol) in THF (390 mL) was added n-BuLi (2.5M, 184 mL) at 0° C. under N₂. The mixture was stirred at 0° C. for 30 minutes, cooled to −78° C., Intermediate 78 (30 g, 0.23 mol) in THF (90 mL) was added to the mixture at −78° C. under N₂. The mixture was stirred at −78° C. for 1.5 hours, then warmed to room temperature NH₄Cl (aq) was added to the mixture, extracted with EtOAc, the organic phase was washed with brine, dried over Na₂SO₄ and concentrated to give a residue, which was purified by silica gel chromatography to give Intermediate 79 (16 g) as a yellow oil.

¹H NMR (CDCl₃, 400 MHz): δ (ppm) 7.48-7.50 (m, 1H), 7.18-7.26 (m, 3H), 5.44 (t, J=2.4 Hz, 1H), 5.03 (t, J=2 Hz, 1H), 2.95-2.99 (m, 2H), 2.77-2.81 (m, 2H).

### Synthesis of Intermediate 80

A mixture of Intermediate 79 (16 g, 0.123 mol) in DCM (330 mL) was added Rh₂(OAc)₄ (0.54 g, 1.23 mmol) at refluxed under N₂. Ethyl diazoacetate (42.1 g, 0.369 mol) in DCM (80 mL) was added to the mixture at refluxed under N₂. The mixture was stirred at refluxed for 3 hours and then the mixture was stirred at RT overnight. The mixture was concentrated to give a residue, which was purified by silica gel chromatography to give Intermediate 80 (23 g) as a yellow oil.

¹H NMR (CDCl₃, DMSO-d₆, 400 MHz): δ (ppm) 7.12-7.25 (m, 7H), 6.71 (d, J=4.8 Hz, 1H), 4.23-4.28 (m, 2H), 4.13-4.19 (m, 2H), 3.01-3.02 (m, 4H), 2.07-1.28 (m, 3H), 2.00-2.06 (m, 4H), 1.61-1.66 (m, 1H), 1.10-1.42 (m, 8H); MS: 217.1 [M+1].

### Synthesis of Intermediate 81

To a solution of Intermediate 80 (18 g, 0.083 mol) in EtOH (180 mL) was added KOH (14 g, 0.25 mol) at room temperature. The mixture was stirred at refluxed for 3 hrs. The mixture was concentrated to give a residue, added water, the pH of the solution was adjusted to 4˜5 with HCl (1 N), extracted with EtOAc, the organic phase was washed with brine, dried over Na₂SO₄ and concentrated to give crude Intermediate 81 (16 g crude) as dark oil.

MS: 187.1 [M+1].

### Synthesis of Intermediate 82 and Intermediate 83

To a solution of Intermediate 81 (16 g, 0.084 mol) in toluene (160 mL) was added TEA (17 g, 0.17 mol) and DPPA (27.5 g, 107 mmol) at 0° C. under N₂. The mixture was stirred at 0° C. for 2 hrs. Then the mixture was poured into ice-water, extracted with EA, the organic phase was washed with water, dried over Na₂SO₄ and concentrated to give a residue, which was dissolved into t-BuOH (120 mL), the mixture was stirred at refluxed for 6 hrs., then the mixture was concentrated to give a residue, purified by prep-HPLC to give Intermediate 82 Trans relative (1.5 g) as a white solid and Intermediate 83 Cis relative (0.8 g) as a white solid.

¹H NMR (CDCl₃, 400 MHz): δ (ppm) 7.17-7.25 (m, 1H), 7.09-7.12 (m, 2H), 6.63-6.65 (m, 1H), 4.74 (br, 1H), 3.02-3.08 (m, 2H), 2.75 (br, 1H), 2.04-2.19 (m, 2H), 1.34-1.43 (m, 10H), 0.91 (br, 1H)

### Synthesis of Intermediate 84

To a solution of Intermediate 82 Trans relative (1.0 g, 3.86 mmol) in EA (6 mL) was added Ethyl Acetate/HCl (10 mL, 8 mol) at room temperature. The mixture was stirred at room temperature for 2 hrs. Then the mixture was concentrated to give crude, which was washed with MTB and filtered to give Intermediate 84 (0.5 g) as a white solid.

¹H NMR (DMSO-d₆, 400 MHz): δ (ppm) 8.64 (s, 3H), 7.22 (d, J 6 Hz, 1H), 7.13-7.16 (m, 2H), 6.82 (t, J=2 Hz, 1H), 3.01-3.06 (m, 2H), 2.78 (t, J=6.4 Hz, 1H), 2.14-2.27 (m, 2H), 1.30 (d, J=6.4 Hz, 2H); MS: 160.1 [M+1].

### Synthesis of Intermediate 85

A mixture of Intermediate 84 (0.5 g, 2.56 mol) in DCE (15 mL) was added tert-butyl N-(2-oxoethyl)carbamate (407.6 mg, 2.56 mmol) at 0° C. under N₂. Then AcOH (153.6 mg, 2.56 mmol) was added to the mixture at 0° C.˜10° C. The mixture was stirred at 0° C. for 1.5 hours, cooled to 0° C. NaBH(Ac)₃ (0.98 g, 4.6 mmol) was added to the mixture at 0° C. under N₂. The mixture was stirred at 0° C. for 1.5 hours. Then the mixture was poured into ice-water, extracted with EtOAc, the organic phase was washed with water, dried over Na₂SO₄ and concentrated to give a residue, which was purified by silica gel chromatography to give Intermediate 85 (300 mg) as a yellow oil.

¹H NMR (CDCl₃, 400 MHz): δ (ppm) 7.12-7.20 (m, 1H), 7.09-7.11 (m, 2H), 6.61-6.63 (m, 1H), 5.30 (br, 1H), 3.25 (d, J=4.4 Hz, 2H), 3.00-3.05 (m, 2H), 2.81-2.85 (m, 2H), 2.28-2.35 (m, 2H), 2.07-2.15 (m, 2H), 1.42 (s, 9H), 1.16-1.19 (m, 1H), 0.84 (t, J=4.4 Hz, 1H); MS: 303.1 [M+1].

### Synthesis of Intermediate 86

To a solution of Intermediate 85 (300 mg, 0.99 mmol) in DCM (4 mL) was added CF₃COOH (2 mL) at room temperature. The mixture was stirred at room temperature for 2 hrs. The mixture was concentrated to give a residue, added water, the pH of the solution was adjusted to 8-9 with Na₂CO₃ (aq), extracted with DCM, the organic phase was washed with brine, dried over Na₂SO₄ and concentrated to give Intermediate 86 (160 mg crude) as a yellow oil.

¹H NMR (CDCl₃, 400 MHz): δ (ppm) 7.18-7.26 (m, 1H), 7.09-7.11 (m, 2H), 6.62-6.64 (m, 1H), 3.00-3.05 (m, 2H), 2.79-2.82 (m, 2H), 2.71-2.77 (m, 2H), 2.28-2.34 (m, 2H), 2.09-2.12 (m, 1H), 1.50 (br s, 3H), 1.14-1.25 (m, 1H), 0.81 (t, J=4.8 Hz, 1H); MS: 203.1 [M+1].

### Synthesis of Compound I-81

To a solution of Compound I-6 (106 mg, 0.40 mmol) in DMSO (5 mL) was added Intermediate 86 (160 mg, 0.79 mmol) and triethylamine (122 mg, 1.2 mmol), then the mixture was stirred at 100° C. for overnight. The mixture was poured into ice water, extracted with DCM/MeOH (10/1), the organic phase was washed with brine, dried over Na₂SO₄ and concentrated to give a residue, which was purified by prep-HPLC to give Compound I-81 trans relative (46 mg, 14.6%) as a yellow solid.

¹H NMR (DMSO-d₆, 400 MHz): δ (ppm) 15.55 (brs, 1H), 8.67 (s, 1H), 8.14 (s, 1H), 7.06-7.15 (m, 3H), 7.04-7.05 (m, 2H), 6.65-6.67 (m, 1H), 6.48 (s, 1H), 6.03 (d, J=8 Hz, 1H), 4.33 (q, J=4 Hz, 2H,), 3.32-3.36 (m, 2H), 2.88-2.93 (m, 2H), 2.81-2.84 (m, 2H), 2.21-2.28 (m, 2H), 2.18-2.20 (m, 1H), 1.32 (t, J=7.2 Hz, 3H), 1.13-1.15 (m, 1H), 0.73 (t, J=4.4 Hz, 1H); MS: 450.9 [M+1].

### Synthesis of Compound I-82

### Synthesis of Intermediate 87

To a solution of Intermediate 83 Cis relative (600 mg, 2.31 mmol) in EtOAc (3 mL) was added HCl/EtOAc (8 M, 6 mL) at room temperature. The mixture was stirred at room temperature for 2 hrs. Then the mixture was concentrated to give a crude product, which was triturated with MTBE, filtered to give Intermediate 87 (450 mg) as a white solid.

¹H NMR (DMSO-d₆, 400 MHz): δ (ppm) 8.32 (brs, 3H), 7.21-7.31 (m, 2H), 7.17-7.20 (m, 2H), 2.85-3.02 (m, 3H), 2.82-2.84 (m, 1H), 2.16-2.20 (m, 1H), 1.91-1.98 (m, 1H), 1.50-1.53 (m 1H), 1.30-1.33 (m, 1H); MS: 160.0 [M+1].

### Synthesis of Intermediate 88

A mixture of Intermediate 87 (450 mg, 2.3 mol) in DME (13.5 mL) was added tert-butyl N-(2-oxoethyl)carbamate (366 mg, 2.3 mmol) at 0° C. under N₂. Then AcOH (138 mg, 2.3 mmol) was added to the mixture at 0-10° C. The mixture was stirred at 0° C. for 1.5 hours, cooled to 0° C. NaBH(OAc)₃ (877 mg, 4.1 mmol) was added to the mixture at 0° C. under N₂. The mixture was stirred at 0° C. for 1.5 hours. Then the mixture was poured into ice-water, extracted with EtOAc, the organic phase was washed with water, dried over Na₂SO₄ and concentrated to give a residue, which was purified by silica gel chromatography to give Intermediate 88 (400 mg) as a yellow oil.

¹H NMR (CDCl₃, 400 MHz): δ (ppm) 7.15-7.22 (m, 1H), 7.12-7.14 (m, 2H), 7.01-7.04 (m, 1H), 4.45 (bs, 1H), 2.94-3.05 (m, 4H), 2.60-2.70 (m, 1H), 2.21-2.36 (m, 3H), 1.82-1.86 (m, 1H), 1.45-1.57 (m, 10H), 1.05-1.26 (m, 1H), 0.97-1.00 (m, 1H); MS: 303.1 [M+1].

### Synthesis of Intermediate 89

To a solution of Intermediate 88 (400 mg, 1.32 mmol) in DCM (10 mL) was added TFA (3 mL) at room temperature. The mixture was stirred at room temperature for 2 hrs. The mixture was concentrated to give a residue, added water, the pH of the solution was adjusted to 8˜9 with Na₂CO₃ (aq), extracted with DCM, the organic phase was washed with brine, dried over Na₂SO₄ and concentrated to give Intermediate 89 (100 mg) as a yellow oil.

¹H NMR (CDCl₃, 400 MHz): δ (ppm) 7.20-7.21 (m, 1H), 7.10-7.13 (m, 2H), 7.04-7.06 (m, 1H), 2.95-3.02 (m, 2H), 2.52-2.61 (m, 3H), 2.23-2.40 (m, 2H), 2.20-2.23 (m, 1H), 1.84-1.87 (m, 1H), 1.41-1.43 (m, 3H), 1.05-1.08 (m, 1H), 0.97-1.00 (m, 1H).

### Synthesis of Compound I-82

To a solution of Compound I-6 (66.5 mg, 0.25 mmol) in DMSO (8 mL) was added Intermediate 89 (100 mg, 0.50 mmol) and triethylamine (75 mg, 0.75 mmol), then the mixture was stirred at 100° C. for overnight. The mixture was poured into ice water, extracted with DCM/MeOH (10/1), the organic phase was washed with brine, dried over Na₂SO₄ and concentrated to give a residue, which was purified by prep-HPLC to give Compound I-82 cis relative (31 mg, 27.6%) as a yellow solid.

¹H NMR (DMSO-d₆, 400 MHz): δ (ppm) 15.55 (brs, 1H), 8.66 (s, 1H), 8.17 (s, 1H), 7.10-7.12 (m, 3H), 7.03-7.05 (m, 1H), 6.96-6.98 (m, 2H), 6.17 (s, 1H), 5.88 (d, J=8 Hz, 1H), 5.75 (1H, s), 4.30 (d, J=8 Hz, 2H), 3.09-3.33 (m, 2H), 2.87-2.91 (m 2H), 2.54-2.63 (m 1H), 2.42-2.44 (m, 2H), 2.41-2.42 (m, 1H), 1.88-1.89 (m, 1H), 1.29 (t, J=7.2 Hz, 3H), 1.02-1.05 (m, 1H), 0.89-0.92 (m, 1H); MS: 451.1 [M+1].

### Example 19: Compound I-83 and 1-84

Synthetic route:

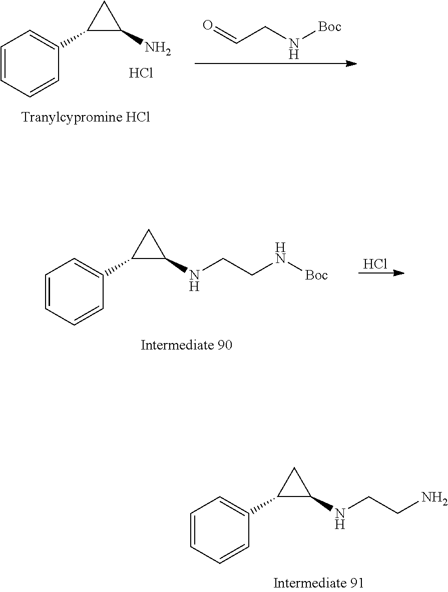 [SMILES: N[C@@H]1C[C@H]1c1ccccc1 ; Cl ; CC(C)(C)OC(=O)NCC=O ; CC(C)(C)OC(=O)NCCN[C@@H]1C[C@H]1c1ccccc1 ; Cl ; NCCN[C@@H]1C[C@H]1c1ccccc1]

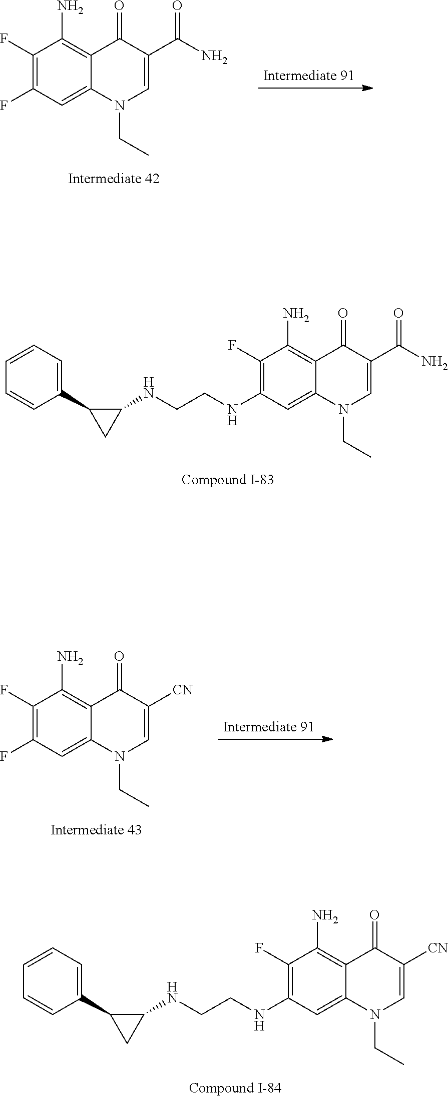 [SMILES: CCn1cc(C(N)=O)c(=O)c2c(N)c(F)c(F)cc21 ; CCn1cc(C(N)=O)c(=O)c2c(N)c(F)c(NCCN[C@@H]3C[C@H]3c3ccccc3)cc21 ; CCn1cc(C#N)c(=O)c2c(N)c(F)c(F)cc21 ; CCn1cc(C#N)c(=O)c2c(N)c(F)c(NCCN[C@@H]3C[C@H]3c3ccccc3)cc21]

### Synthesis of Intermediate 90

To a solution of tranylcypromine HCl trans relative (3.0 g, 17.8 mmol) in DCE (90 mL) was added tert-butyl 2-oxoethylcarbamate (2.8 g, 17.8 mmol) under N₂ at 0° C., AcOH (1.1 g, 17.8 mmol) was added, the mixture was stirred at 0° C. for 1 hour. Then NaBH(OAc) (6.8 g, 32 mmol) was added slowly. The mixture was stirred at 0-10° C. for 1 hour, then room temperature for 3 hrs, poured into ice water, extracted with EtOAc, the organic phase was concentrated in vacuum, purified by prep-HPLC to give Intermediate 90 trans relative (1.2 g, crude) as a yellow oil.

### Synthesis of Intermediate 91

To a solution of Intermediate 90 trans relative (1.2 g, crude) in DCM (12 mL) was added HCl/dioxane (12 mL, 7 mol/L), the mixture was stirred at room temperature for 2 hrs. The mixture was concentrated, ice water was added, adjusted pH=8˜9 with Na₂CO₃ (aq), extracted with DCM/MeOH=10/1. The organic phase was dried over Na₂SO₄ and concentrated in vacuum to give Intermediate 91 trans relative (0.7 g, crude) as a yellow oil.

### Synthesis of Compound I-83

To a solution of Intermediate 42 (0.2 g, 0.75 mmol) in DMSO (8 mL) was added Et₃N (0.3 g, 2.3 mmol), then Intermediate 91 trans relative (0.4 g, 2.3 mmol) was added. The mixture was stirred at 75° C. for 16 hrs, cooled. Then the mixture was poured into ice-water, filtered, the crude was purified by prep-HPLC to give product Compound I-83 trans relative (15 mg, 4.7%) as a yellow solid.

¹H NMR (CD₃OD, 400 MHz): δ (ppm) 8.51 (s, 1H), 7.23-7.27 (m, 2H), 7.17-7.19 (m, 1H), 7.08-7.10 (m, 2H), 5.96 (d, J=6.8 Hz, 1H), 4.18-4.20 (m, 2H), 3.55-3.58 (m, 2H), 3.26-3.28 (m, 2H), 2.71-2.73 (m, 1H), 2.20-2.22 (m, 1H), 1.41-1.44 (m, 3H), 1.25-1.33 (m, 1H), 1.21-1.23 (m, 1H); MS: 424.1 [M+1].

### Synthesis of Compound I-84

To a solution of Intermediate 43 (0.4 g, 1.6 mmol) in DMSO (16 mL) was added Et % N (0.5 g, 4.8 mmol), then Intermediate 91 trans relative (0.8 g, 2.3 mmol) was added. The mixture was stirred at 75° C. for 16 hrs, cooled. Then the mixture was poured into ice-water, filtered, the crude was purified by prep-HPLC to give Compound I-84 trans relative (34 mg, 5.2%) as a yellow solid.

¹H NMR (CD₃OD, 400 MHz): δ (ppm) 8.44 (s, 1H), 8.14 (s, 1H), 7.09-7.25 (m, 5H), 5.84-5.85 (m, 1H), 4.08-4.11 (bm, 2H), 3.60-3.62 (bm, 2H), 3.38-3.40 (m, 2H), 2.86-2.89 (m, 1H), 2.45-2.46 (m, 1H), 1.48-1.51 (m, 1H), 1.34-1.37 (m, 3H), 1.26-1.27 (m, 1H); MS: 406.3 [M+1].

### Example 20: Synthesis of Compound I-86

Synthetic route:

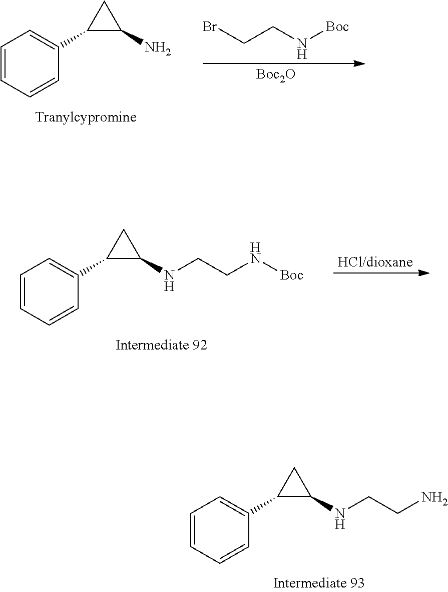 [SMILES: "Boc2O" ; N[C@@H]1C[C@H]1c1ccccc1 ; CC(C)(C)OC(=O)NCCBr ; CC(C)(C)OC(=O)NCCN[C@@H]1C[C@H]1c1ccccc1 ; "HCl/dioxane" ; NCCN[C@@H]1C[C@H]1c1ccccc1]

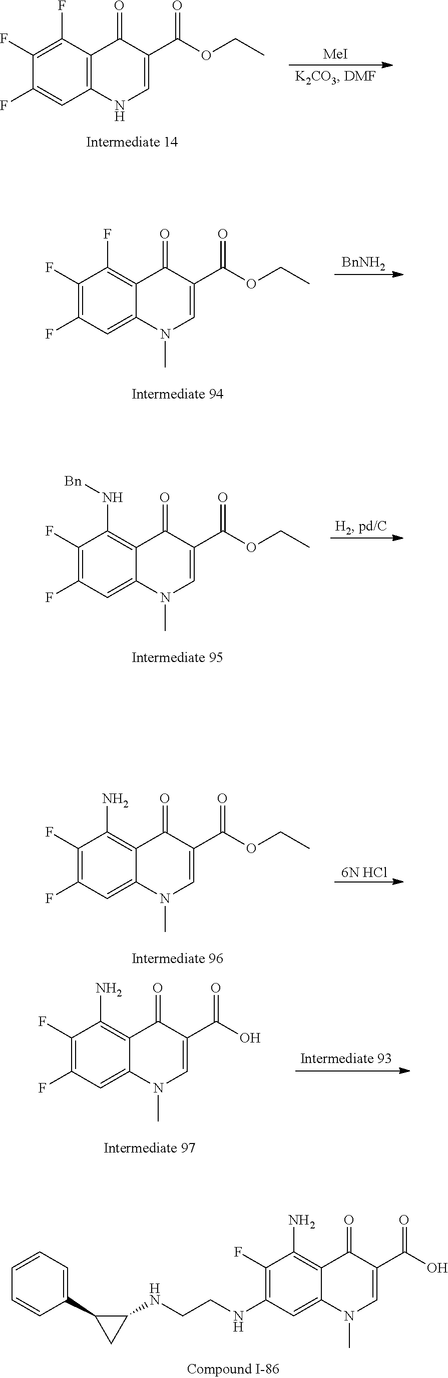

### Synthesis of Intermediate 92

To a solution of tranylcypromine HCL trans relative (6.0 g, 45.0 mmol) in DMF (100 mL) was added tert-butyl 2-bromoethylcarbamate (15.0 g, 67.5 mmol), K₂CO₃ (15.6 g, 112.8 mmol), the mixture was stirred at 60° C. for 2 hrs. After cooled to room temperature then Boc₂O (10.8 g, 49.5 mmol) was added slowly. The mixture was stirred at 30° C. for 16 hrs, poured into ice water, extracted with EtOAc, the organic phase was washed with water, brine, dried over Na₂SO₄, concentrated in vacuum, purified by silica gel chromatography to give Intermediate 92 trans relative (3.8 g, 30.4%) as a white solid.

¹H NMR (CDCl₃, 400 MHz): δ (ppm) 7.24-7.26 (m, 2H), 7.12-7.16 (m, 1H), 7.01-7.03 (m, 2H), 4.87 (s, 1H), 3.23-3.24 (m, 2H), 2.85 (t, J=6.0 Hz, 2H), 2.32-2.35 (m, 1H), 1.86-1.90 (m, 2H), 1.50 (s, 9H), 0.96-1.07 (m, 2H).

### Synthesis of Intermediate 93

To a solution of Intermediate 92 trans relative (3.0 g, 10.9 mmol) in DCM (30 mL) was added HCl/dioxane (30 mL, 7 mol/L), the mixture was stirred at room temperature for 2 hrs. The mixture was concentrated, ice water was added, adjusted pH=8˜9, extracted with DCM/MeOH=10/1, the organic phase was washed brine, dried over Na₂SO₄ and concentrated in vacuum to give a crude of Intermediate 93 trans relative (2.6 g crude, 100%) as a yellow oil.

¹H NMR (CDCl₃, 400 MHz): δ (ppm) 7.22-7.26 (m, 2H), 7.12-7.16 (m, 1H), 7.02-7.04 (m, 2H), 2.77-2.82 (m, 4H), 2.32-2.35 (m, 1H), 1.86-1.91 (m, 1H), 0.96-1.09 (m, 2H).

### Synthesis of Intermediate 94

To a solution of Intermediate 14 (8 g, 29 mmol) in DMF (80 mL) was added K₂CO₃ (12.0 g, 58 mmol) and iodomethane (8.2 g, 58 mmol). The mixture was stirred at 50° C. for 3 hrs. Then the mixture was poured into ice-water, filtered to give the solid, which was triturated with MTBE, filtered to give Intermediate 94 (6.4 g, 77.4%) as a white solid.

¹H NMR (DMSO-d₆, 400 MHz): δ (ppm) 8.62 (s, 1H), 7.73-7.78 (m, 1H), 4.21 (q, J=7.2 Hz, 14.4 Hz, 2H), 3.84 (s, 3H), 1.27 (t, J=7.2 Hz, 3H); MS: 285.9 [M+1].

### Synthesis of Intermediate 95

To a solution of Intermediate 94 (6.4 g, 22 mmol) in toluene (128 mL) was added benzylamine (3.6 g, 34 mmol) and triethylamine (4.4 g, 44 mmol). The mixture was stirred at 100° C. for 3 hrs. TLC (D/M=20/1) showed the reaction was complete. Then the mixture was poured into ice-water, extracted with DCM, the organic phase was washed with brine, dried over Na₂SO₄ and concentrated to give a residue, which was triturated with MTBE, filtered to give Intermediate 95 (6.5 g, 78%) as a white solid.

¹H NMR (CDCl₃, 400 MHz): δ (ppm) 11.10 (brs, 1H), 8.27 (s, 1H), 7.36-7.38 (m, 2H), 7.28-7.32 (m, 2H), 7.23-7.25 (m, 1H), 6.17-6.21 (m, 1H), 4.71-4.73 (m, 2H), 4.37 (q, J=7.2 Hz, 14.4 Hz, 2H), 3.68 (s, 3H), 1.38 (t, J=7.2 Hz, 3H); MS: 372.9 [M+1].

### Synthesis of Intermediate 96

To a solution of Intermediate 95 (6.5 g, 17 mmol) in EtOH (65 mL) and acetic acid (65 mL) was added Pd/C (1.0 g, 10% wt). The mixture was stirred at 45° C. for 5 hrs under H2 at 1 atm. TLC (D/M=20/1) showed the reaction was complete. Then the mixture was filtered, concentrated to give a crude, which was triturated with MTBE (100 mL), filtered to give Intermediate 96 (4.5 g, 93.7%) as a white solid.

¹H NMR (CDCl₃, 400 MHz): δ (ppm) 8.32 (s, 1H), 6.23˜6.27 (m, 1H), 4.38 (q, J=7.2 Hz, 14 Hz, 2H), 3.72 (s, 3H), 1.39 (t, J=7.2 Hz, 3H); MS: 282.9 [M+1].

### Synthesis of Intermediate 97

A mixture of Intermediate 96 (4.5 g, 16 mmol) in HCl (6N, 45 mL) was stirred at 100° C. for 6 hrs, cooled. Then the mixture was filtered to give Intermediate 97 (2.0 g, 49.4%) as a white solid.

¹H NMR (DMSO-d₆, 400 MHz): δ (ppm) 14.78 (br s, 1H), 8.78-8.80 (m, 1H), 7.91 (br s, 2H), 6.85-6.90 (m, 1H), 3.86 (s, 3H); MS: 255.0 [M+1].

### Synthesis of Compound I-83

To a solution of Intermediate 97 (1.8 g, 7.1 mmol) in DMSO (50 mL) was added Intermediate 93 (3.15 g, 17.8 mmol) and triethylamine (2.15 g, 21.3 mmol), then the mixture was stirred at 75° C. for 10 hrs. The mixture was poured into ice water, filtered, the filtered cake was triturated with DMSO/MTBE=1/6 to give Compound I-86 trans relative (313 mg, 10.8%) as off-white solid.

¹H NMR (DMSO-d₆, 400 MHz): δ (ppm) CO₂H peak was omitted, 8.67 (s, 1H), 7.01-7.23 (m, 7H), 6.45 (s, 1H), 5.95-5.96 (m, 1H), 3.77 (s, 2H), 3.32 (s, 3H), 2.87 (t, 2H), 2.29-2.31 (m, 1H), 1.78-1.80 (m, 1H), 0.92-1.00 (m, 211); MS: 411.0 [M+1].

### Example 21: Synthesis of Compound I-87

Synthetic route:

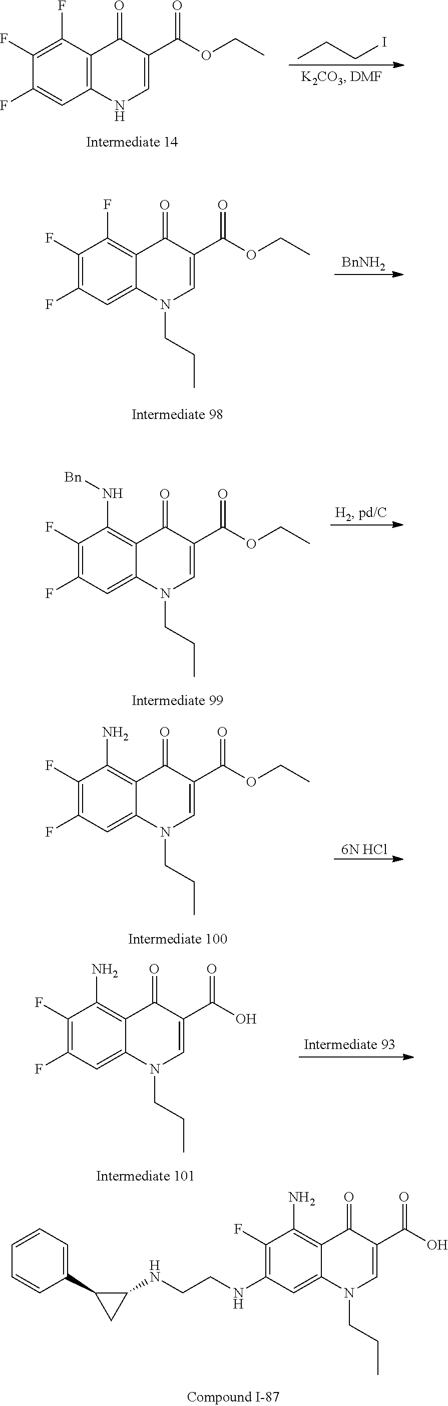

### Synthesis of Intermediate

To a solution of Intermediate 14 (12.0 g, 44 mmol) in DMF (240 mL) was added K₂CO₃ (18.2 g, 132 mmol) and 1-iodopropane (15.0 g, 88 mmol). The mixture was stirred at 65° C. for 2 hrs. Then the mixture was poured into ice-water, filtered to give the solid, which was triturated with MTBE, filtered to give Intermediate 98 (6.0 g, 43.2%) as a white solid.

¹H NMR (CDCl₃, 400 MHz): δ (ppm) 8.36 (s, 1H), 6.98-7.03 (m, 1H), 4.38 (q, J=7.2 Hz, 14.4 Hz, 2H,), 4.04 (t, J=7.6 Hz, 3H), 1.91 (q, J=7.6 Hz, 14.8 Hz, 2H), 1.40 (t, J=7.2 Hz, 3H), 1.04 (t, J=7.2 Hz, 3H); MS: 313.9 [M+1].

### Synthesis of Intermediate 99

To a solution of Intermediate 98 (6.0 g, 19.2 mmol) in toluene (120 mL) was added benzylamine (3.1 g, 28.8 mmol) and triethylamine (3.9 g, 38.4 mmol). The mixture was stirred at 100° C. for 3 hrs. TLC (D/M=20/1) showed the reaction was complete. Then the mixture was poured into ice-water, extracted with DCM, the organic phase was washed with brine, dried over Na₂SO₄ and concentrated to give a residue. The residue was triturated with MTBE and filtered to give Intermediate 99 (6.0 g, 77.9%) as a white solid.

¹H NMR (CDCl₃, 400 MHz): δ (ppm) 11.13 (br s, 1H), 8.26 (s, 1H), 7.36-7.38 (m, 2H), 7.28-7.32 (m, 2H), 7.22-7.25 (m, 1H), 6.18-6.23 (m, 1H), 4.70-4.73 (m, 2H), 4.37 (q, J=7.2 Hz, 14.4 Hz, 2H), 3.94 (t, J=7.2 Hz, 2H), 1.85-1.90 (m, 2H), 1.38 (t, J=7.2 Hz, 3H), 1.00 (t, J=7.2 Hz, 3H).

### Synthesis of Intermediate 100

To a solution of Intermediate 99 (6.0 g, 15 mmol) in EtOH (60 mL) and acetic acid (60 mL) was added Pd/C (1.2 g, 10% wt). The mixture was stirred at 45° C. for 3 hrs under H₂ at 1 atm. TLC (D/M=20/1) showed the reaction was complete. Then the mixture was filtered, concentrated to give a crude product, which was triturated with MTBE (100 mL), filtered to give Intermediate 100 (4.0 g, 86.0%) as a white solid.

¹H NMR (CDCl₃, 400 MHz): δ (ppm) 8.32 (s, 1H), 6.24-6.29 (m, 1H), 4.39 (q, J=7.2 Hz, 14.4 Hz, 2H), 3.98 (t, J=7.2 Hz, 2H), 1.87-1.92 (m, 2H), 1.40 (t, J=7.2 Hz, 3H), 1.02 (t, J=7.2 Hz, 3H); MS: 310.9 [M+1].

### Synthesis of Intermediate 101

A mixture of Intermediate 100 (4.0 g, 12.9 mmol) in HCl (6 N, 40 mL) was stirred at 100° C. for 6 hrs. The mixture was filtered to give Intermediate 101 (2.0 g, 55.6%) as a white solid.

¹H NMR (DMSO-d₆, 400 MHz): δ (ppm) 14.80 (brs, 1H), 8.70 (s, 1H), 7.91 (brs, 1H), 6.92-6.95 (brs, 2H), 4.19-4.27 (m, 2H), 1.70-1.75 (m, 2H), 0.89 (t, J=7.2 Hz, 3H); MS: 283.1 [M+1].

### Synthesis of Compound I-87

To a solution of Intermediate 101 (1.7 g, 6.03 mmol) in DMSO (50 mL) was added Intermediate 93 trans relative (2.11 g, 12.06 mmol) and triethylamine (1.83 g, 18.09 mmol), then the mixture was stirred at 75° C. for 10 hrs. The mixture was poured into ice water, filtered, the filtered cake was triturated with DCM/MTBE=1/6 to give Compound I-87 trans relative (280 mg, 10.6%) as off-white solid.

¹H NMR (DMSO-d₆, 400 MHz): δ (ppm) CO₂H peak was omitted, 8.65 (s, 1H), 7.08-7.22 (m, 5H), 6.99-7.00 (m, 2H), 6.48 (s, 1H), 6.00-6.01 (m, 1H), 4.27 (s, 2H), 3.32 (s, 2H), 2.85 (t, J=6.4 Hz, 2H), 2.26-2.28 (m, 1H), 1.71-1.80 (m, 3H), 0.94-0.97 (m, 2H), 0.84-0.88 (m, 3H); MS: 439.0 [M+1].

### Example 22: Synthesis of Compound I-88

Synthetic route:

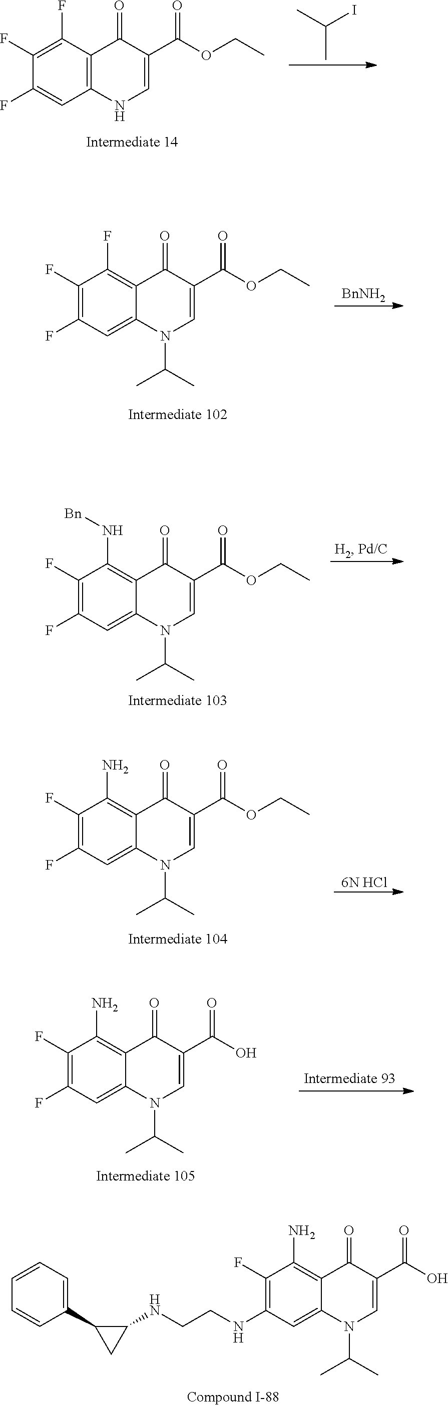

### Synthesis of Intermediate 102

To a solution of Intermediate 14 (20 g, 74 mmol) in DMSO (400 mL) was added K₂CO₃ (31 g, 222 mmol) and 2-iodopropane (25.0 g, 148 mmol). The mixture was stirred at 75° C. for 3 hrs. Then the mixture was poured into ice-water, extracted with EtOAc, the organic phase was washed with brine, dried over Na₂SO₄ and concentrated in vacuum. The crude product was purified by silica gel of chromatography to give Intermediate 102 (1.5 g, 6.5%) as a white solid.

¹H NMR (CDCl₃, 400 MHz): δ (ppm) 8.54 (s, 1H), 7.15-7.20 (m, 1H), 4.64-4.67 (m, 1H), 4.37-4.24 (m, 2H), 1.59-1.61 (m, 6H), 1.37-1.43 (m, 3H); MS: 314.0 [M+1].

### Synthesis of Intermediate 103

To a solution of Intermediate 102 (1.5 g, 4.8 mmol) in toluene (30 mL) was added benzylamine (0.77 g, 7.2 mmol) and triethylamine (1.45 g, 14.4 mmol). The mixture was stirred at 100° C. for 3 hrs. TLC (D/M=20/1) showed the reaction was complete. Then the mixture was poured into ice-water, extracted with DCM, the organic phase was washed with brine, dried over Na₂SO₄ and concentrated to give a residue, which was triturated with MTBE, filtered to give Intermediate 103 (500 mg, 26%) as a white solid.

¹H NMR (CDCl₃, 400 MHz): δ(ppm) 11.24 (brs, 1H), 8.44 (s, 1H), 7.36-7.38 (m, 2H), 7.28-7.32 (m, 2H), 7.22-7.25 (m, 1H), 6.35-6.39 (m, 1H), 4.71-4.73 (m, 2H), 4.56-4.59 (m, 1H), 4.39 (q, J=6.8 Hz, 14 Hz, 2H), 1.54-1.58 (m, 6H), 1.38-1.41 (m, 3H); MS: 401.0 [M+1].

### Synthesis of Intermediate 104

To a solution of Intermediate 103 (500 mg, 1.25 mmol) in EtOH (5 mL) and acetic acid (5 mL) was added Pd/C (100 mg, 10% wt). The mixture was stirred at 45° C. for 3 hrs under H₂ at 1 atm. TLC (D/M=2011) showed the reaction was complete. Then the mixture was filtered, concentrated to give a crude, which was triturated with MTBE (10 mL), filtered to give Intermediate 104 (250 mg, 64.1%) as a white solid.

MS: 310.9 [M+1].

### Synthesis of Intermediate 105

A mixture of Intermediate 104 (270 mg, 0.87 mmol) in HCl (6 N, 3 mL) was stirred at 100° C. for 6 hrs, cooled. Then the mixture was filtered to give Intermediate 105 (120 mg, 49%) as a white solid.

¹H NMR (DMSO-d₆, 400 MHz): δ (ppm) 14.79 (s, 1H), 8.70 (s, 1H), 7.98 (br, 1H), 6.18-6.23 (m, 1H), 4.97-5.00 (m, 1H), 1.49 (d, J=6.4 Hz, 6H); MS: 283.1 [M+1].

### Synthesis of Compound I-88

To a solution of Intermediate 105 (100 mg, 0.35 mmol) in DMSO (6 mL) was added Intermediate 93 (186 mg, 1.05 mmol) and triethylamine (106 mg, 1.05 mmol), then the mixture was stirred at 75° C. for 10 hrs. The mixture was poured into ice water, filtered, the filtered cake was triturated with DCM/MTBE=1/6 to give Compound I-88 trans relative (66 mg, 43.1%) as off-white solid.

¹H NMR (DMSO-d₆, 400 MHz): δ (ppm) 15.6 (brs, 1H), 8.53 (s, 1H), 7.18-7.22 (m, 4H), 7.08-7.11 (m, 1H), 7.00-7.02 (m, 2H), 6.46 (s, 1H), 6.17-6.19 (m, 1H), 4.92-4.95 (m, 1H), 3.30 (s, 2H), 2.85 (t, J=6.4 Hz, 2H), 2.27-2.30 (m, 1H), 1.77-1.81 (m, 1H), 1.47 (d, J=6.4 Hz, 6H), 0.94-0.99 (m, 2H); MS: 438.9 [M+1].

### Example 23: Synthesis of Compound I-89

Synthetic route:

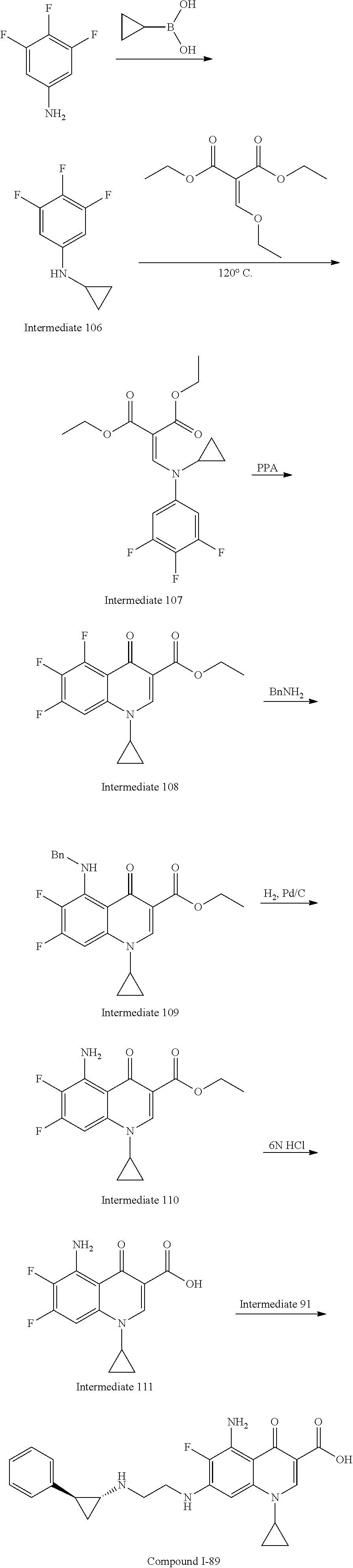

### Synthesis of Intermediate 106

To a solution of 3,4,5-trifluoroaniline (50 g, 0.34 mol) in DCE (500 mL) was added 2,2′-Bipyridine (64.0 g, 0.41 mol), Cyclopropylboronic acid (58.5 g, 0.68 mol), Cu(OAc)₂ (74.5 g, 0.41 mol) and Na₂CO₃ (72.08 g, 0.68 mol). The mixture was stirred at 70° C. for 3.5 hrs. Then the mixture was filtered to give the organic phase, the organic phase was concentrated to give a residue, which was purified by silica gel chromatography to give Intermediate 106 (10.0 g, 15.6%) as a yellow oil.

¹H NMR (CDCl₃, 400 MHz): δ (ppm) 6.30-6.35 (m, 2H), 4.18 (s, 1H), 2.34-2.37 (m, 1H), 0.72-0.77 (m, 2H), 0.48-0.51 (m, 2H); MS: 188.0 [M+1].

### Synthesis of Intermediate 107

A mixture of Intermediate 106 (10 g, 53.5 mmol) and diethyl 2-(ethoxymethylene) malonate (13.9 g, 64.2 mmol) was stirred at 120° C. for overnight, then cooled, MTBE was added, filtered to give Intermediate 107 (12 g, 62.8%) as off-white solid.

¹H NMR (CDCl₃, 400 MHz): δ (ppm) 7.68 (s, 1H), 6.84-6.88 (m, 2H), 4.11-4.24 (m, 4H), 3.03 (br s, 1H), 1.25-1.41 (m, 7H), 0.86-0.91 (m, 3H); MS: 358.0 [M+1].

### Synthesis of Intermediate 108

A mixture of Intermediate 107 (12 g, 33.6 mmol) in Polyphosphoric Acid (PPA) (36 g) was stirred at 140° C. for 3 hrs. Then the mixture was poured into ice-water, then the mixture was filtered to give a solid. The solid was washed with water, and dried to give Intermediate 108 (3.5 g, 33.7%) as a yellow solid.

¹H NMR (DMSO-d₆, 400 MHz): δ (ppm) 8.50 (s, 1H), 7.51-7.55 (m, 1H), 4.38 (q, J=7.2 Hz, 14 Hz, 2H), 3.35-3.37 (m, 1H), 1.34-1.41 (m, 5H), 1.11-1.15 (m, 2H); MS: 312.0 [M+1].

### Synthesis of Intermediate 109

To a solution of Intermediate 108 (3.5 g, 11.3 mmol) in toluene (70 mL) was added benzylamine (1.2 g, 17.0 mmol) and triethylamine (3.4 g, 33.9 mmol). The mixture was stirred at 100° C. for 3 hrs. The mixture was poured into ice-water, extracted with DCM, the organic phase was washed with brine, dried over Na₂SO₄ and concentrated. Trituration with MTBE and filtration afforded Intermediate 109 (3.5 g, 77.8%) as a white solid.

¹H NMR (CDCl₃, 400 MHz): δ (ppm) 11.00 (br s, 1H), 8.40 (s, 1H), 7.32-7.37 (m, 2H), 7.26-7.30 (m, 2H), 7.22-7.24 (m, 1H), 6.72-6.76 (m, 1H), 4.70-4.73 (m, 2H), 4.37 (q, J=7.2 Hz, 14.0 Hz, 2H), 3.25-3.28 (m, 1H), 1.38 (t, J=7.2 Hz, 3H), 1.26-1.31 (m, 2H), 1.04-1.07 (m, 2H); MS: 399.0 [M+1].

### Synthesis of Intermediate 110

To a solution of Intermediate 109 (3.5 g, 8.8 mmol) in EtOH (35 mL) and acetic acid (35 mL) was added Pd/C (0.7 g, 10% wt). The mixture was stirred at 45° C. for 3 hrs under H₂ at 1 atm. Then the mixture was filtered, concentrated to give a crude, which was triturated with MTBE (50 mL), and filtered to give Intermediate 110 (1.8 g, 66.7%) as a white solid.

¹H NMR (CDCl₃, 400 MHz): δ (ppm) 8.45 (s, 1H), 6.76-6.81 (m, 1H), 4.39 (q, J=6.8 Hz, 13.6 Hz, 2H), 3.29-3.30 (m, 1H), 1.39 (t, J=6.8 Hz, 3H), 1.29-1.31 (m, 2H), 1.09 (s, 2H); MS: 309.1 [M+1].

### Synthesis of Intermediate 111

A mixture of Intermediate 110 (1.8 g, 5.8 mmol) in HCl (6N, 18 mL) was stirred at 100° C. for 6 hrs and then cooled. Then the mixture was filtered and washed with water to give Intermediate 111 (0.9 g, 56%) as a white solid.

¹H NMR (DMSO-d₆, 400 MHz): δ (ppm) 14.65 (s, 1H), 8.60 (s, 1H), 7.95 (br, 2H), 7.16-7.21 (m, 1H), 3.60-3.63 (m, 1H), 1.25-1.28 (m, 2H), 1.16-1.24 (m, 2H); MS: 298.1 [M+1].

### Synthesis of Compound I-89

To a solution of Intermediate 111 (0.9 g, 3.24 mmol) in DMSO (30 mL) was added Intermediate 93 trans relative (1.1 g, 6.48 mmol) and triethylamine (0.98 g, 9.72 mmol), then the mixture was stirred at 75° C. for 12 hrs. The mixture was poured into ice water, filtered, the filtered cake was triturated with DCM/MTBE=1/6 to give Compound I-89 trans relative (290 mg, 20.7%) as a white solid.

¹H NMR (DMSO-d₆, 400 MHz): δ (ppm) 8.40 (s, 1H), 7.25-7.28 (m, 2H), 7.16-7.19 (m, 2H), 7.11-7.13 (m, 2H), 6.67 (s, 1H), 6.41 (d, J=5.6 Hz, 1H), 3.47-3.51 (m, 3H), 3.19-3.21 (m, 2H), 2.81-2.82 (m, 1H), 2.21-2.23 (m, 1H), 1.30-1.32 (m, 1H), 1.18-1.22 (m, 3H), 1.03-1.06 (m, 2H); MS: 437.0 [M+1].

### Example 24: Synthesis of Compound I-90

Synthetic route:

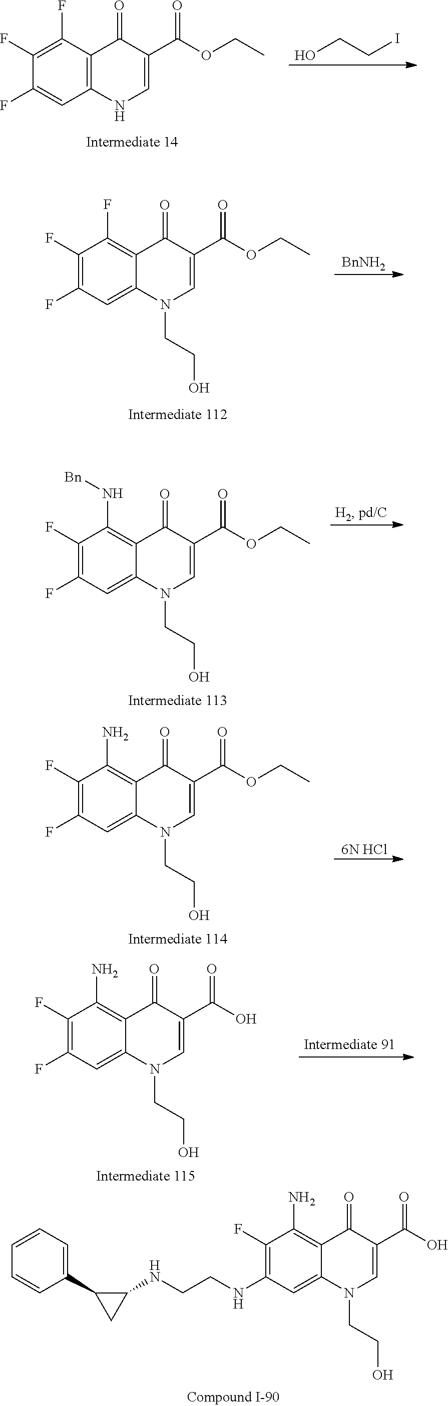

### Synthesis of Intermediate 112

To a solution of Intermediate 14 (6.0 g, 22 mmol) in DMF (120 mL) was added NaH (1.8 g, 44 mmol) and Iodoethanol (7.6 g, 44 mmol). The mixture was stirred at 95° C. for overnight. Then the mixture was poured into ice-water, filtered to give the solid, which was triturated with MTBE, filtered to give Intermediate 112 (4.5 g, 64.9%) as a white solid.

¹H NMR (DMSO-d₆, 400 MHz): δ (ppm) 8.51 (s, 1H), 7.88-7.95 (m, 1H), 5.00 (t, J=5.2 Hz, 1H), 4.36-4.38 (m, 2H), 3.67-3.70 (m, 2H), 1.25-1.29 (m, 3H); MS: 315.9 [M+1].

### Synthesis of Intermediate 113

To a solution of Intermediate 112 (4.5 g, 14 mmol) in toluene (67 mL) was added phenylmethanamine (2.25 g, 21 mmol) and triethylamine (4.2 g, 42 mmol). The mixture was stirred at 100° C. for 3 hrs. Then the mixture was poured into ice-water, extracted with DCM, the organic phase was washed with brine, dried over Na₂SO₄ and concentrated to give a residue, which was triturated with MTBE, filtered to give Intermediate 113 (4.0 g, 71.4%) as a white solid.

¹H NMR (CDCl₃, 400 MHz): δ (ppm) 11.21 (s, 1H), 8.21 (s, 1H), 7.31-7.37 (m, 4H), 7.28-7.30 (m, 1H), 5.98-6.03 (m, 1H), 5.09 (br s, 1H), 4.53-4.55 (m, 2H), 4.23 (q, J=7.2 Hz, 14.4 Hz, 2H), 3.99-4.01 (m, 2H), 3.54 (s, 2H), 1.30 (t, J=6.8 Hz, 3H); MS: 403.0 [M+1].

### Synthesis of Intermediate 114

To a solution of Intermediate 113 (5.0 g, 1.25 mmol) in EtOH (50 mL) and acetic acid (50 mL) was added Pd/C (1.0 g, 10% wt). The mixture was stirred at 45° C. for 3 hrs under H₂ at 1 atm. Then the mixture was filtered, concentrated to give a crude, which was triturated with MTBE (20 mL), and filtered to give Intermediate 114 (2.2 g, 58.8%) as a white solid.

### Synthesis of Intermediate 115

A mixture of Intermediate 114 (2.2 g, 7.05 mmol) in HCl (6 N, 22 mL) was stirred at 100° C. for 6 hrs and then cooled. Then the mixture was filtered to give Intermediate 115 (1.0 g, 50%) as a white solid.

¹H NMR (DMSO-d₆, 400 MHz): δ (ppm) 14.77 (brs, 111), 8.71 (s, 111), 7.88 (br, 2H), 7.05-7.10 (m, 1H), 5.00 (br, 1H), 4.44 (m, 2H), 3.70 (m, 211); MS: 285.1 [M+1].

### Synthesis of Compound I-90

To a solution of Intermediate 115 (1.0 g, 3.5 mmol) in DMSO (30 mL) was added Intermediate 93 trans relative (1.24 g, 7.0 mmol) and triethylamine (1.1 g, 10.5 mmol), then the mixture was stirred at 75° C. for 10 hrs. The mixture was poured into ice water, filtered, the filtered cake was triturated with DCM/MTBE=1/6 to give Compound I-90 trans relative (347 mg, 22.5%) as off-white solid.

¹H NMR (DMSO-d₆, 400 MHz): δ (ppm) CO₂H peak was omitted, 8.50 (s, 1H), 7.08-7.22 (m, 5H), 7.00-7.03 (m, 2H), 6.05 (br s, 1H), 5.95 (br s, 1H), 4.19-4.25 (m, 2H), 3.70 (s, 2H), 3.28 (s, 2H), 2.84 (s, 2H), 2.25 (br s, 1H), 1.80 (br s, 1H), 0.94-0.99 (m, 2H); MS: 441.1 [M+1].

### Example 25: Synthesis of Compound I-91

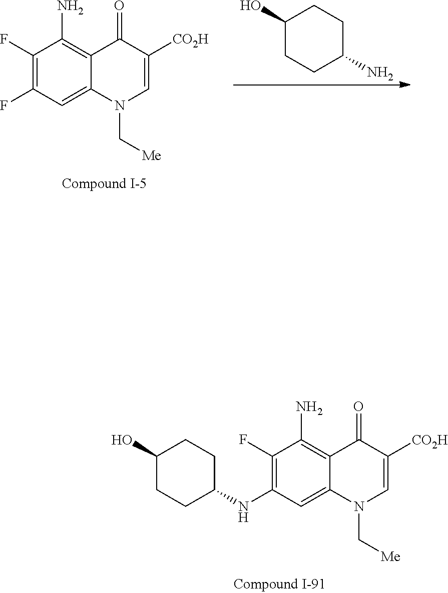 [SMILES: CCn1cc(C(=O)O)c(=O)c2c(N)c(F)c(F)cc21 ; N[C@H]1CC[C@H](O)CC1 ; CCn1cc(C(=O)O)c(=O)c2c(N)c(F)c(N[C@H]3CC[C@H](O)CC3)cc21]

In a sealable vial, a mixture of Compound I-5 (0.48 g, 1.79 mmol) and trans-4-aminocyclohexanol (0.62 g, 5.38 mmol) in DMSO (4.5 mL) was flushed with N₂, sealed and heated at 100° C. for 16 h. The suspension was cooled to RT, diluted with water (10 mL) and filtered. The resulting solid was dissolved into DMSO (30 mL at 100° C.), treated with water (˜5 mL), cooled and filtered to give Compound I-91 (0.53 g, 82°/o) as light yellow solid. ¹H NMR (500 MHz, DMSO-d₆) 1.84-1.87 (4H, m), 3.32-3.49 (˜2H including H₂O, m), 4.36 (2H, m), 4.57 (1H, bs), 6.03 (1H, bd, J=4.5 Hz), 6.18 (1H, bd, J=6.5 Hz), 7.13 (2H, bs) and 8.65 (1H, s); MS (APCI) m/z 364 (M+1, 12%) and 157 (100%).

### Example 26: Synthesis of Compound I-92

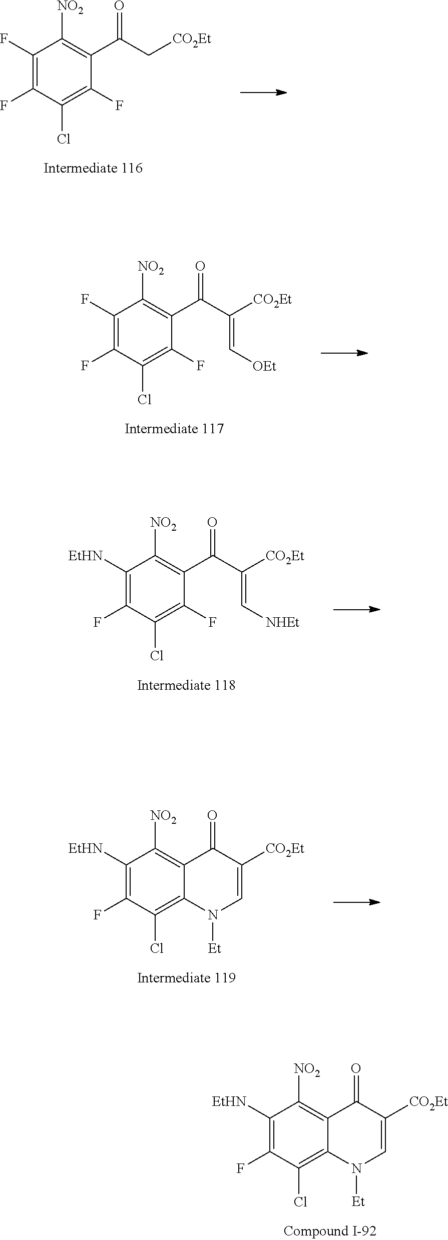

### Synthesis of Intermediate 117

A solution of Intermediate 116 (0.88 g, 2.70 mmol, prepared according to Domagala, J. M. et al. J. Med. Chem. 1991, 34, 1142-1154), triethyl orthoformate (0.67 mL, 4.02 mmol) and acetic anhydride (0.64 mL, 6.78 mmol) was heated at 120° C. under N₂ for 4 h then RT overnight. The mixture was concentrated and vacuum dried to give Intermediate 117 (1.20 g, >100%) as dark brown-red oil, which was used without purification. MS (APCI) m/z 334/336 (M-47, 100/35%).

### Synthesis of Intermediate 118

A solution of Intermediate 117 in CH₃CN (27 mL) was treated with 10% ethylamine in THE solution (2.40 g, 5.32 mmol). The sample was stirred at RT under N₂ for 1 h, concentrated then chromatographed (SiO₂, 25% EtOAc in hexanes) to give Intermediate 118 (0.51 g, 47%) as orange solid. MS (APCI) m/z 406/408 (M+1, 100/35%).

### Synthesis of Intermediate 119

A mixture of Intermediate 118 (0.51 g, 1.34 mmol) and potassium carbonate (0.37 g, 2.68 mmol) in CH₃CN was refluxed under N₂ for 30 min. The mixture was concentrated then partitioned between CHCl₃ and sat. KH₂PO₄ solution (25 mL of each). The organic extract was washed with sat. NaCl solution (25 mL), dried (MgSO₄), filtered, concentrated and chromatographed (SiO₂, 50% EtOAc in hexanes) to give Intermediate 119 (0.33 g, 63%) as orange solid. MS (APCI) m/z 386/388 (M+1, 100/37%).

### Synthesis of Compound I-92

In a sealable vial, a mixture of Intermediate 119 0.27 g, 0.70 mmol) and 6 N HCl (2 mL, 12 mol) in EtOH (2 mL) was flushed with N₂, sealed and heated at 100° C. for 4 hrs then RT overnight. The sample was filtered, washed with water and vacuum dried to give Compound I92 (0.20 g, 80%) as orange solid. ¹H NMR (500 MHz, DMSO-d₆) t), 1.42 (3H, t), 3.21-3.24 (2H, m), 4.85 (2H, q), 6.17 (1H, bs) and 8.89 (1H, s); MS (APCI) m/z 358/360 (M+1, 100/32%).

¹H NMR (499 MHz, CD₃OD) δ 8.49 (s 1H), 4.29-4.16 (m, 2H), 3.79 (t, J=10.2 Hz, 2H), 1.96 (dd, J=12.3, 3.0 Hz, 2H), 1.74 (dd, J=9.6, 3.8 Hz, 2H), 1.61 (dd, J=9.1, 3.8 Hz, 1H), 1.36 (m, 1H), 1.29-1.12 (m, 3H); MS (APCI) m/z: 332.52

### Example 28. Biological Activity of Compounds of the Present Disclosure

Assay:

Enzymatic activities of compounds of the present disclosure can be tested using recombinant human GSK3 in an in vitro enzymatic assay to determine the ability of the compounds of the present disclosure to inhibit GSK3α and GSK3β.

Exemplary enzyme and substrate assay concentrations are shown in Table I below.

TABLE I

| | Enzyme Used | |
|---|---|---|
| Assay | (ng)/Reaction | Substrate/ATP |
| GSK3α | 13 | 0.1 mg/ml GSKtide/10 μM ATP |
| GSK3β | 13 | 0.1 mg/ml GSKtide/10 μM ATP |

Assay Conditions:

The assay was performed using Kinase-Glo® Max luminescence kinase assay kit (Promega). The assay measures kinase activity by quantitating the amount of ATP remaining in solution following a kinase reaction. The luminescent signal from the assay is correlated with the amount of ATP present and is inversely correlated with the amount of kinase activity.

To perform the assay, compounds were diluted in 10% DMSO and 5 μl of the dilution was added to a 50 μl reaction so that the final concentration of DMSO was 1% in all of reactions. The enzymatic reactions were conducted at 30° C. for 40 minutes. The 50 μl reaction mixture contained 40 mM Tris, pH 7.4, 10 mM MgCl₂, 0.1 mg/ml BSA, 2 mM DTT, 0.1 mg/ml GSK tide substrate, 10 uM ATP and GSK3. After the enzymatic reaction, 50 μl of Kinase-Glo Max Luminescence kinase assay solution (Promega) was added to each reaction and plates were incubated for 15 minutes at room temperature. Luminescence signal was measured using a BioTek Synergy 2 microplate reader.

Data Analysis:

Kinase activity assays were performed in duplicate at each concentration. The luminescence data were analyzed using the computer software, GraphPad Prism. The difference between luminescence intensities in the absence of Kinase (Lut) and in the presence of Kinase (Luc) was defined as 100% activity (Lut-Luc). Using luminescence signal (Lu) in the presence of the compound, percent activity was calculated as: % activity={(Lut-Lu)/(Lut-Luc)}×100%, where Lu=the luminescence intensity in the presence of the compound (all percent activities below zero were shown zero in the table).

The values of percent activity versus a series of compound concentrations were then plotted using non-linear regression analysis of Sigmoidal dose-response curve generated with the equation Y=B+(T−B)/1+10((LogEC50−X)×Hill Slope), where Y=percent activity, B=minimum percent activity, T=maximum percent activity, X=logarithm of compound and Hill Slope=slope factor or Hill coefficient. The ICs, value was determined by the concentration causing a half-maximal percent activity.

Compounds of the present disclosure were synthesized and tested for GSK3alpha and GSK3beta activity. Activity of exemplary compounds is shown in Table II below.

TABLE II

| | Potency | | Potency | |
|---|---|---|---|---|
| Compound | GSK3alpha | | GSK3beta | |
| I-1 | 3.6 | μM | 6.7 | μM |
| I-3 | 250 | nM | 366 | nM |
| I-7 | 38 | nM | 18 | nM |
| I-8 | 713 | nM | 452 | nM |
| I-9 | 69 | nM | 53 | nM |
| I-10 | 3.8 | μM | 1.9 | μM |
| I-11 | 846 | nM | 990 | nM |
| I-12 | 82 | nM | 55 | nM |
| I-13 | 628 | nM | 555 | nM |
| I-14 | >10,000 | nM | >10,000 | nM |
| I-15 | 352 | nM | 223 | nM |
| I-16 | 107 | nM | 68 | nM |
| I-17 | 659 | nM | 2030 | nM |
| I-18 | 121 | nM | 231 | nM |
| I-19 | 19 | nM | 29 | nM |
| I-20 | 102 | nM | 338 | nM |
| I-21 | 392 | nM | 334 | nM |
| I-22 | 137 | nM | 115 | nM |
| I-23 | 221 | nM | 770 | nM |
| I-26 | >10 | μM | >10 | μM |
| I-27 | 47 | nM | 75 | nM |
| I-28 | 451 | nM | 691 | nM |
| I-29 | 119 | nM | 303 | nM |
| I-32 | 1600 | nM | 2100 | nM |
| I-38 | 6.3 | nM | 2.5 | nM |
| I-50 | >10 | μM | >10 | μM |
| I-52 | >10 | μM | >10 | μM |
| I-55 | >10 | μM | >10 | μM |
| I-56 | >10 | μM | >10 | μM |
| I-57 | 762 | nM | 1762 | nM |
| I-58 | >10 | μM | >10 | μM |
| I-59 | 4676 | nM | 4432 | nM |
| I-60 | 32 | nM | 31 | nM |
| I-61 | 72 | nM | 268 | nM |
| I-62 | 449 | nM | 504 | nM |
| I-63 | >10 | μM | >10 | μM |
| I-64 | >10 | μM | >10 | μM |
| I-65 | >10 | μM | >10 | μM |
| I-66 | 2.6 | nM | 2.5 | nM |
| I-67 | 2.0 | nM | 1.9 | nM |
| I-68 | 581 | nM | 711 | nM |
| I-69 | 61 | nM | 52 | nM |
| I-70 | 207 | nM | 169 | nM |
| I-81 | 457 | nM | 546 | nM |
| I-82 | 1550 | nM | 3382 | nM |
| I-83 | 3.3 | nM | 3.4 | nM |
| I-84 | 2.7 | nM | 2.2 | nM |
| I-86 | 919 | nM | 2165 | nM |
| I-87 | 114 | nM | 157 | nM |
| I-88 | 19.5 | nM | 83 | nM |
| I-89 | 393 | nM | 308 | nM |
| I-90 | 990 | nM | 1599 | nM |
| I-93 | 3951 | nM | 6540 | nM |

*The values shown in the table are approximate values.

These data demonstrate that the compounds of the present disclosure are useful for inhibiting GSK3α and GSK3β, which further demonstrates that the compounds of the present disclosure are useful for the treatment or prevention of GSK3α and GSK3β-mediated diseases or disorders as well as diseases or disorders where GSK3α and GSK3β plays a role.

Assay:

Enzymatic activities of compounds of the present disclosure can be tested with recombinant human LSD using an in vitro enzymatic assay to determine the ability of the compounds of the present disclosure to inhibit LSD-1 and LSD2. Examples of the assay are provided below.

Exemplary enzyme and Substrate assay concentrations are shown in Table III below.

TABLE III

| | Enzyme Used | |
|---|---|---|
| Assay | (ng)/Reaction | Substrate |
| LSD-1: A-screen assay | 80 | Biotinylated histone H3 peptide |
| LSD2: A-screen assay | 120 | Biotinylated histone H3 peptide |

Assay Conditions:

All of the enzymatic reactions were conducted in duplicate at room temperature for 60 minutes in a 10 μL mixture containing assay buffer, histone H3 peptide substrate, demthylase enzyme, and the test compound. These 10 μL reactions were carried out in wells of a 384-well Optiplate (PerkinElmer). The dilution of the compounds was first performed in 100% DMSO with the highest concentration at 5 mM Each intermediate compound dilution (in 100% DMSO) was then directly diluted 30× fold into assay buffer for 3.3× concentration (DMSO). Enzyme only and blank only wells had a final DMSO concentration of 1%. From this intermediate step, 3 μL of compound was added to 4 μL of demethylase enzyme dilution was incubated for 30 minutes at room temperature. After this incubation, 3 μL of peptide substrate was added. The final DMSO concentration was 1%. After enzymatic reactions, 5 μL of anti-Mouse or anti-Rabbit Acceptor beads (PerkinElmer, diluted 1:500 with 1× detection buffer) and μL of Primary antibody (BPS, diluted 1:800 with 1× detection buffer) were added to the reaction mix. After brief shaking, the plate was incubated for 30 minutes. Finally, 10 μL of AlphaScreen™ Streptavidin-conjugated donor beads (Perkin, diluted 1:125 with 1× detection buffer) were added. After 30 minutes, the samples were measured in an AlphaScreen microplate reader (EnSpire Alpha 2390 Multilabel Reader, Perkin Elmer).

Data Analysis

Enzyme activity assays were performed in duplicates at each concentration. The A-screen intensity data were analyzed and compared. In the absence of the compound, the intensity in each data set was defined as 100% (Ce) activity. In the absence of enzyme, the intensity in each data set was defined as 0% (C0) activity. The percent activity in the presence of each compound was calculated according to the following equation: % activity=(C−C₀)/(Ce−C0), where C=the intensity in the presence of the compound.

The value of percent activity versus a series of compound concentrations was then plotted using non-linear regression analysis of Sigmoidal dose-response curve generated with the equation Y=B+(T−B)/1+10((LogEC50−X)×Hill Slope), where Y=percent activity, B=minimum percent activity, T=maximum percent activity, X=logarithm of compound and Hill Slope=slope factor or Hill coefficient. The IC₅₀ value was determined by the concentration causing a half-maximal percent activity.

Compounds of the present disclosure were synthesized and tested for LSD-1 and LSD-2 activity. Activity of exemplary compounds is shown in Table IV below.

TABLE IV

| | Potency LSD-1 | Potency LSD-2 |
|---|---|---|
| | % Inhibition at | % Inhibition at |
| Compound | 50 μM or IC₅₀ | 50 μM or IC₅₀ |
| I-1 | 10% @ 50 μM | 6% |
| I-3 | 13% @ 50 μM | 0% |
| I-7 | 10% @ 50 μM | 5% |
| I-8 | 10% @ 50 μM | 1% |
| I-9 | 6% @ 50 μM | 3% |
| I-10 | 22% @ 50 μM | 6% |
| I-11 | 27% @ 50 μM | 13% |
| I-12 | 36% @ 50 μM | 0% |
| I-13 | 26% @ 50 μM | 18% |
| I-14 | 5% @ 50 μM | 3% |
| I-15 | 2% @ 50 μM | 2% |
| I-16 | 32% @ 50 μM | 5% |
| I-17 | 2% @ 50 μM | 19% |
| I-18 | 11% @ 50 μM | 1% |
| I-19 | 27% @ 50 μM | 17% |
| I-20 | <10% @ 50 μM | 10% |
| I-21 | 19% @ 50 μM | 0% |
| I-22 | 11% @ 50 μM | 2% |
| I-23 | 26% @ 50 μM | 16% |
| I-26 | 1.5 μM | 53% |
| I-27 | 1.6 μM | 45% |
| I-28 | 1.3 μM | 65% |
| I-29 | 6.9 μM | 54% |
| I-32 | 40% @ 50 μM | 36% |
| I-38 | 15% @ 50 μM | 0% |
| I-50 | 5.5 μM | 50% |
| I-52 | 0% @ 50 μM | 3% |
| I-56 | 56% @ 50 μM | 28% |
| I-57 | 83% @ 50 μM | 42% |
| I-58 | 77% @ 50 μM | 7% |
| I-59 | 11% @ 50 μM | 24% |
| I-60 | 15% @ 50 μM | 4% |
| I-61 | 26% @ 50 μM | 15% |
| I-62 | >100 μM | 49 μM |
| I-63 | 26% @ 50 μM | 6% |
| I-64 | 72% @ 50 μM | 2% |
| I-65 | 88% @ 50 μM | 38% |
| I-66 | 6% @ 50 μM | 4% |
| I-67 | 8% @ 50 μM | 4% |
| I-68 | 0% @ 50 μM | 96% |
| I-69 | 17% @ 50 μM | 3% |
| I-70 | 1.1 μM | >100 μM |
| I-81 | 0.058 μM | 43 μM |
| I-82 | 0.19 μM | 76% |
| I-83 | 0.23 μM | >100 μM |
| I-84 | 1.4 μM | 41 μM |
| I-86 | 1.9 μM | 6% |
| I-87 | 3.3 μM | 15% |
| I-88 | 2.6 μM | 14% |
| I-89 | 7.4 μM | 5% |
| I-90 | 8.5 μM | 8% |
| I-93 | 3% @ 50 μM | 2% |

*The values shown in the table are approximate values.

These data demonstrate that the compounds of the present disclosure are useful for inhibiting LSD-1 and LSD2, which further demonstrates that the compounds of the present disclosure are useful for the treatment or prevention of LSD-1 and LSD2-mediated diseases or disorders as well as diseases or disorders where LSD-1 and LSD2 plays a role.

Foxo-1 potency of exemplary compounds can be found in literature (“Discovery of Novel Forkhead Box O1 Inhibitors for Treating Type 2 Diabetes: Improvement of Fasting Glycemia in Diabetic db/db Mice” Molecular Pharmacology (2010), 78(5), 961-970) and are shown in Table V below.

TABLE V

| Compound | Potency Foxo-1 |
|---|---|
| I-3 | <100 nM |
| I-7 | <100 nM |

*The values shown in the table are approximate values.

These data demonstrate that Compounds I-3 and I-7 are useful for inhibiting Foxo-1. This further demonstrates that the compounds of the present disclosure are useful for the treatment or prevention of Foxo-1-mediated diseases or disorders as well as diseases or disorders where Foxo-1 plays a role.

Assay: Lgr5+

The compounds of the present disclosure were used in Lgr5-EGFP-IRES-Cre-ER mice (Barker et al., 2007) to analyze the effects of small molecules on cochlear stem cell expansion.

Isolation of stem cells from the inner ear: All animal studies were conducted under an approved institutional protocol according to National Institutes of Health guidelines. For experiments with neonatal mice (postnatal days 1-3), the cochleae were dissected in Hank's Balanced Salt Solution (HBSS) and the organs of Corti were separated from the stria vascularis and the modiolus. The organs of Corti were then treated with Cell Recovery Solution (Corning) for 1 hour to separate cochlear epithelium from the underlying mesenchyme. Epithelia were then collected and treated with TrypLE (Life Technologies) for 15-20 minutes at 37° C. Single cells obtained by mechanical trituration were filtered (40 μm) and suspended in Matrigel® (Corning) for 3D culture.

Expansion of Lgr5-Positive Cells

Cells were cultured in a 1:1 mixture of DMEM and F12, supplemented with Glutamax (GIBCO), N₂, B27 (Invitrogen), EGF (50 ng/ml; Chemicon), bFGF (50 ng/ml; Chemicon), IGF1 (50 ng/ml; Chemicon) and the composition provided herein. Medium was changed every other day.

Differentiation of Lgr5-Positive Progenitor Cells

To test whether compounds can be useful in compositions for expansion and induction of differentiation of Lgr5-positive progenitor cells, stem cell colonies were differentiated in a 1:1 mixture of DMEM and F12, supplemented with Glutamax (GIBCO), N₂, B27 (Invitrogen), with addition of specific drugs or after removal of growth factors without drug addition. Small molecules such as compounds provided herein were added to the culture to test their effect on differentiation.

Analysis

Lgr5-positive cells were quantified after 10 days (D10) in culture in multiple conditions. Cell colonies were dissociated into single cells using TrypLE (Gibco). The cells were then stained with propidium iodide (P1) and were analyzed using a flow cytometer for Lgr5-GFP expression. The number of GFP-positive cells and the percentage of GFP-positive cells were quantified.

Atoh1-nGFP-positive cells were quantified at day 0 (D0) and day 10 (D10) of differentiation treatment to determine the number of hair cells that have differentiated. Cell colonies were incubated in Cell Recovery Solution to release the colonies from Matrigel and dissociated into single cells using TrypLE. The total number and percentage of GFP-positive cells were quantified using a flow cytometer for multiple culture conditions. ANOVA was used to compare means across conditions, and the two-tailed Student's T-test was used to compare each condition to the treatment with the highest yield.

Cell number and cell enrichment can also be quantified using imaging, where cell area reflects cell number and cell area percent reflects enrichment of the cell type of interest. Cell types of interest can include supporting cells and/or progenitor cells.

Compounds of the present disclosure were synthesized and tested for Lgr5+ activity as described above. Activity of exemplary compounds is shown in Table VI below.

TABLE VI

| | | | % of Lgr5+ |
|---|---|---|---|
| Compound | Potency | | Positive Cells |
| I-1 | >10 | μM | <5% |
| I-3 | >10 | μM | <5% |
| I-7 | 370 | nM | 22% |
| I-8 | 3.3 | μM | 21% |
| I-9 | 1.1 | μM | 13% |
| I-10 | >10 | μM | 1% |
| I-11 | 3.3 | μM | 17% |
| I-12 | 1.1 | μM | 25% |
| I-15 | 3.3 | μM | 30% |
| I-16 | 1.1 | μM | 22% |
| I-17 | 3.3 | μM | 22% |
| I-18 | 1.1 | μM | 31% |
| I-19 | 370 | nM | 17% |
| I-20 | 1.1 | μM | 42% |
| I-21 | 3.3 | μM | 21% |
| I-22 | 1.1 | μM | 16% |
| I-23 | 2.2 | μM | 14% |
| I-26 | >10 | μM | <5% |
| I-27 | 1.1 | μM | 26% |
| I-28 | 8 | μM | 40% |
| I-29 | 4 | μM | 38% |
| I-38 | 123 | nM | 7.5% |
| I-57 | 30 | μM | 1% |
| I-60 | 370 | nM | 30% |
| I-61 | 1.1 | μM | 30% |
| I-69 | 1.1 | μM | 25% |
| I-70 | 3.3 | μM | 30% |
| I-81 | 10 | μM | 65% |
| I-82 | 10 | μM | 48% |
| I-83 | 1.1 | μM | 12% |
| I-84 | 1.1 | μM | 9% |
| I-86 | >30 | μM | 4% |
| I-87 | 3.3 | μM | 52% |
| I-88 | 1.1 | μM | 32% |
| I-89 | 3.3 | μM | 35% |
| I-93 | >30 | μM | 8% |

*The values shown in the table are approximate values.

These data demonstrate that the compounds of the present disclosure are useful for the expansion of Lgr5+ cells, e.g., cochlear Lgr5+ cells, and accordingly, can be useful for increasing hair cell number and/or improving hearing in a subject.

Compounds I-7, I-20, I-28, and I-29 were further assessed for their ability to generate Lgr5 GFP+ progenitor cell proliferation and enrichment of Lgr5 GFP+ cochlear progenitor cells in a background of growth factors.

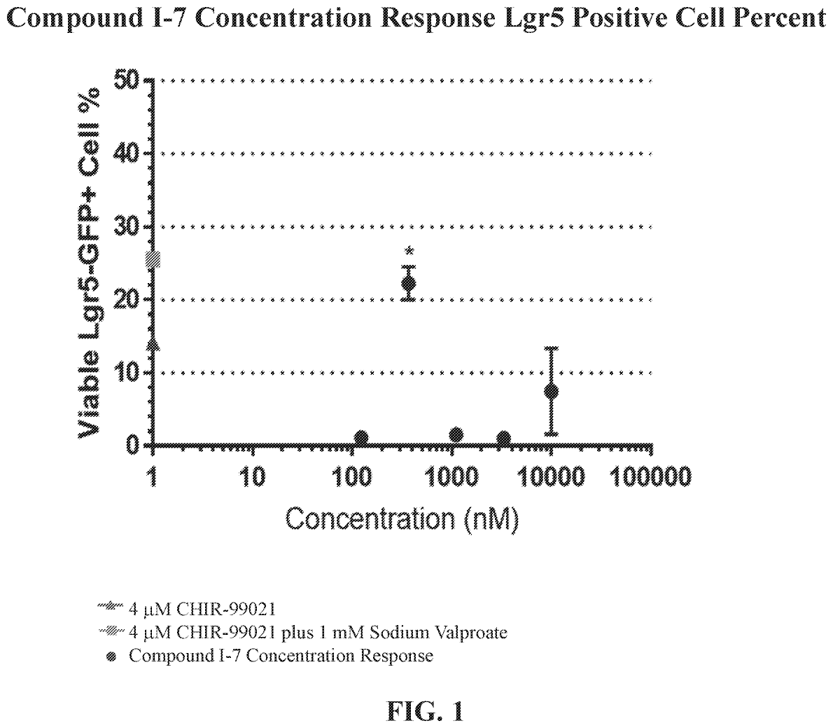

Following the Lgr5+ assay described above, the concentration response percent of Lgr5 positive cells for Compound I-7 was compared to CHIR-99021 (4 μM) and the combination of CHIR-99021 (4 μM) and sodium valproate (1 mM) See FIG. 1).

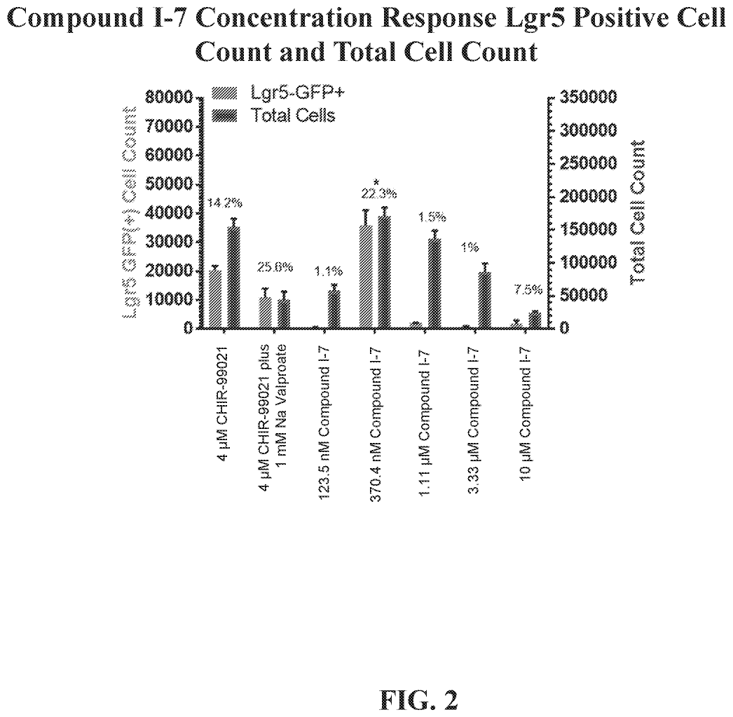

The concentration response Lgr5 positive cell count and total cell count for Compound I-7 at concentrations of 123.5 nM, 370.4 nM, 1.11 μM, 3.33 μM, and 10 μM was compared to CHIR-99021 (4 μM) and the combination of CHIR-99021 (4 μM) and sodium valproate (1 mM) See FIG. 2.

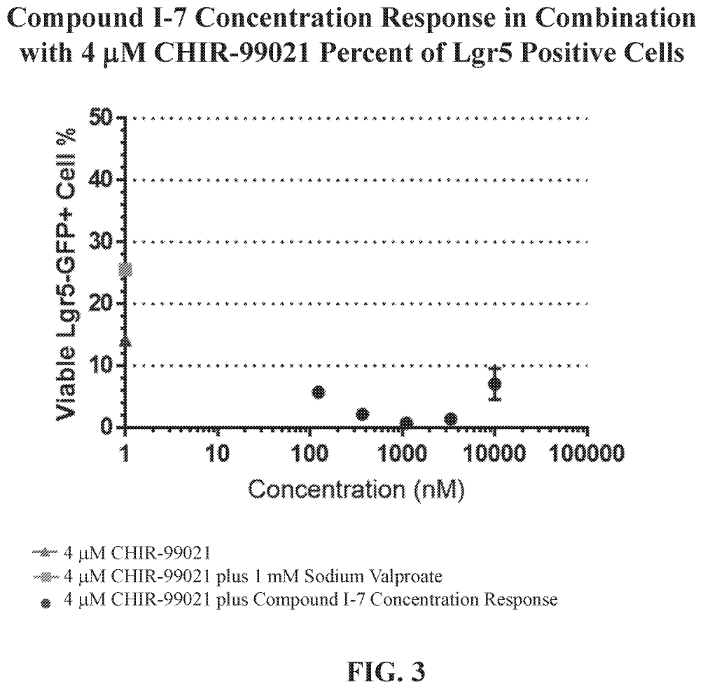

The concentration response percent of Lgr5 positive cells for Compound I-7 in combination with CHIR-99021 (4 μM) was compared to CHIR-99021 (4 μM) alone, and CHIR-99021 (4 μM) in combination with sodium valproate (1 mM) See FIG. 3.

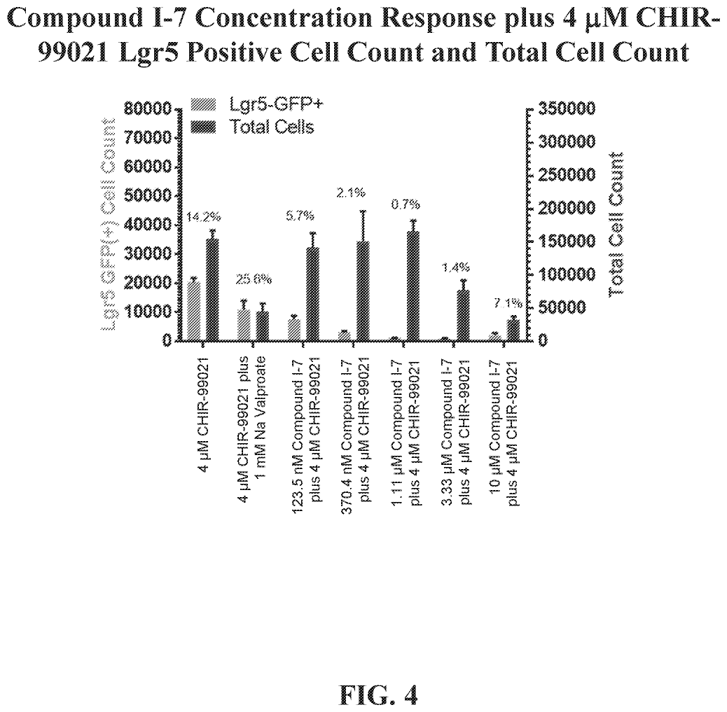

The concentration response Lgr5 positive cell count and total cell count for Compound I-7 at concentrations of 123.5 nM, 370.4 nM, 1.11 μM, 3.33 μM, and 10 μM in combination with CHIR-99021 (4 μM) was compared to CHIR-99021 (4 μM) alone, and CHIR-99021 (4 μMin combination with sodium valproate (1 mM) See FIG. 4.

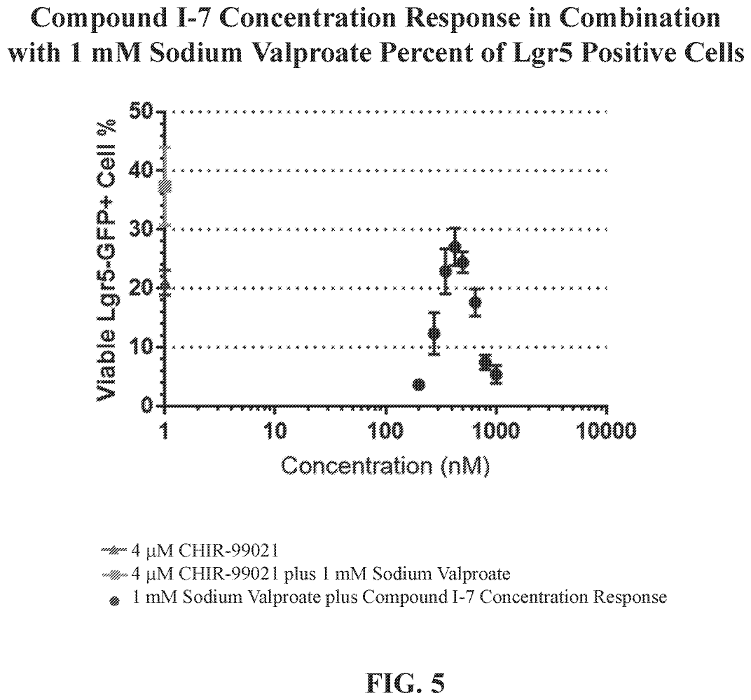

The concentration response percent of Lgr5 positive cells for Compound I-7 in combination with sodium valproate (1 mM) was compared to CHIR-99021 (4 μM) alone, and CHIR-99021 (4 μM) in combination with sodium valproate (1 mM) See FIG. 5.

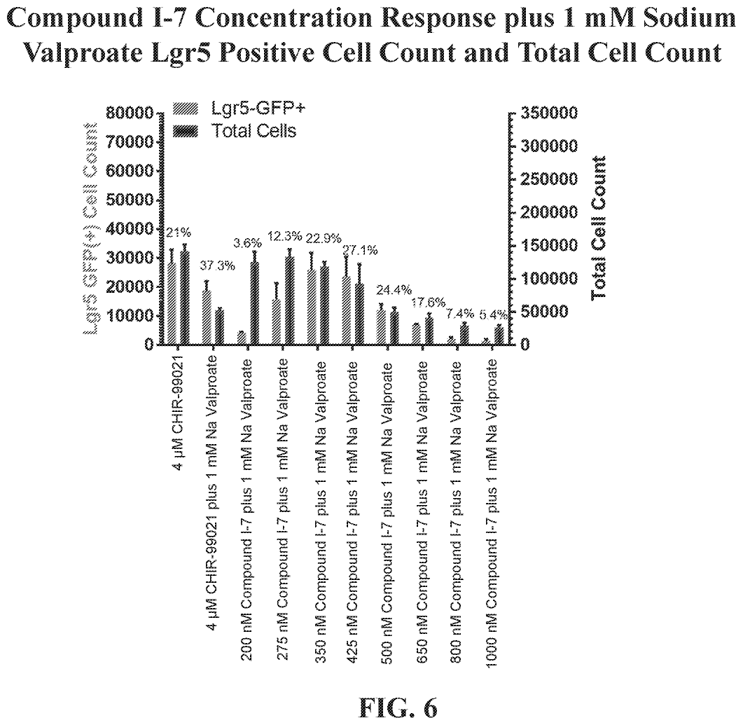

The concentration response Lgr5 positive cell count and total cell count for Compound I-7 at concentrations of 200 nM, 275 nM, 350 nM, 425 nM, 500 nM, 650 nM, 800 nM, and 1000 nM in combination with sodium valproate (1 mM) was compared to CHIR-99021 (4 μM) alone, and CHIR-99021 (4 μM) in combination with sodium valproate (1 mM). See FIG. 6.

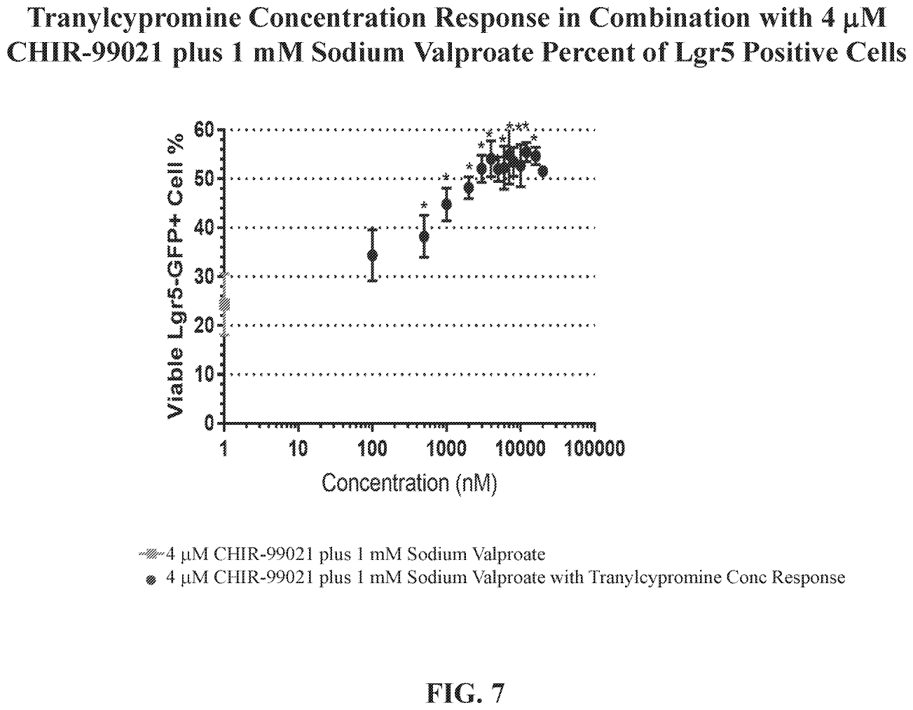

The concentration response percent of Lgr5 positive cells for tranylcypromine in combination with CHIR-99021 (4 μM) and sodium valproate (1 mM) was compared to the combination of CHIR-99021 (4 μM) and sodium valproate (1 mM) See FIG. 7.

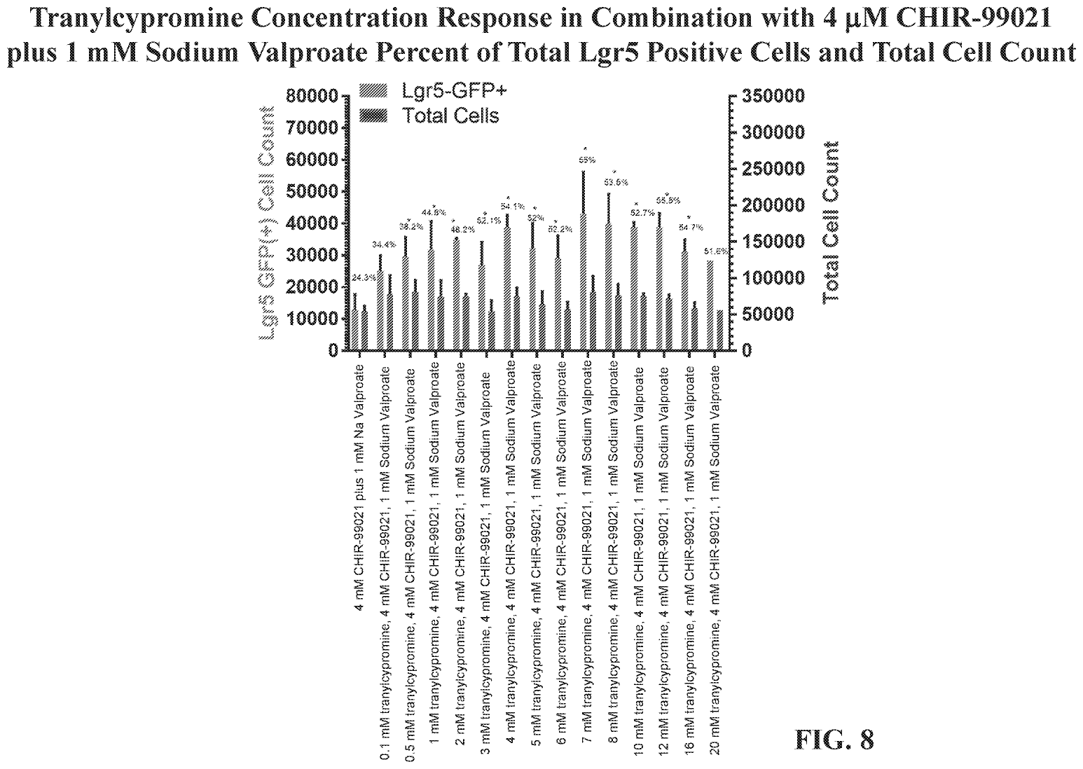

The concentration response Lgr5 positive cell count and total cell count for tranylcypromine at concentrations of 0.1 μM, 0.5 μM, 1 μM, 2 μM, 3 μM, 4 μM, 5 μM, 6 μM, 7 μM, 8 μM, 10 μM, 12 μM, 16 μM, and 20 μM in combination with sodium valproate (1 mM) was compared to CHIR-99021 (4 μM) alone, and CHIR-99021 (4 μM) in combination with sodium valproate (1 mM). See FIG. 8.

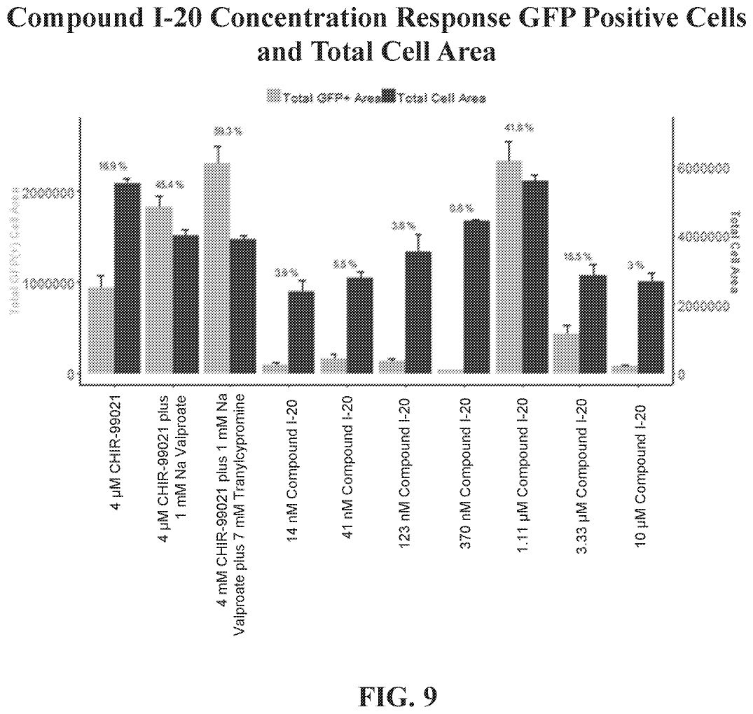

The concentration response for GFP positive cells and total cell area for Compound I-20 at concentrations of 14 nM, 41 nM, 123 nM, 370 nM, 1.11 μM, 3.33 μM, and 10 μM was compared to CHIR-99021 (4 μM) alone, CHIR-99021 (4 μM) in combination with sodium valproate (1 mM), and the combination of CHIR-99021 (4 μM), sodium valproate (1 mM), and tranylcypromine (7 μM). See FIG. 9.

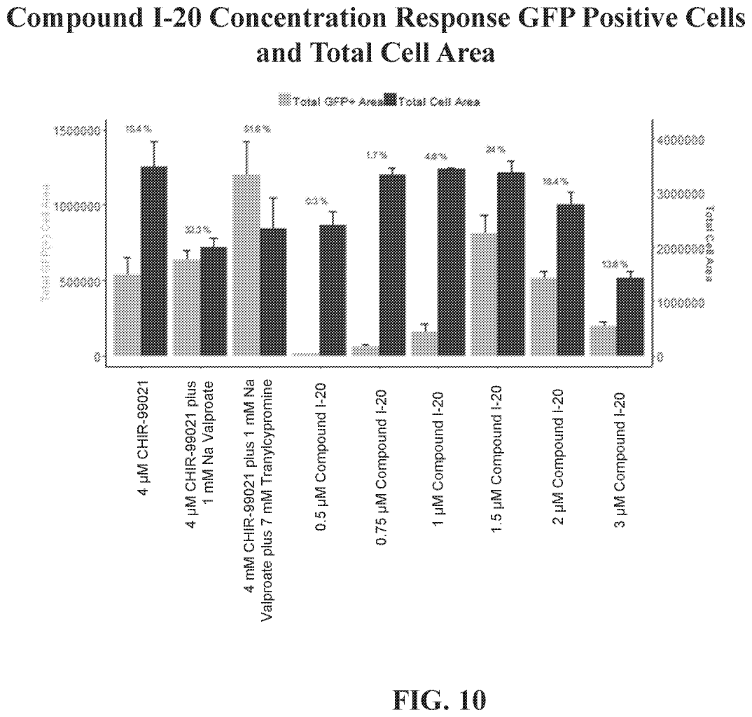

The concentration response for GFP positive cells and total cell area for Compound I-20 at concentrations of 0.5 μM, 0.75 μM, 1 μM, 1.5 μM, 2 μM, and 3 μM was compared to CHIR-99021 (4 μM) alone, CHIR-99021 (4 μM) in combination with sodium valproate (1 mM), and the combination of CHIR-99021 (4 μM), sodium valproate (1 mM), and tranylcypromine (7 μM). See FIG. 10.

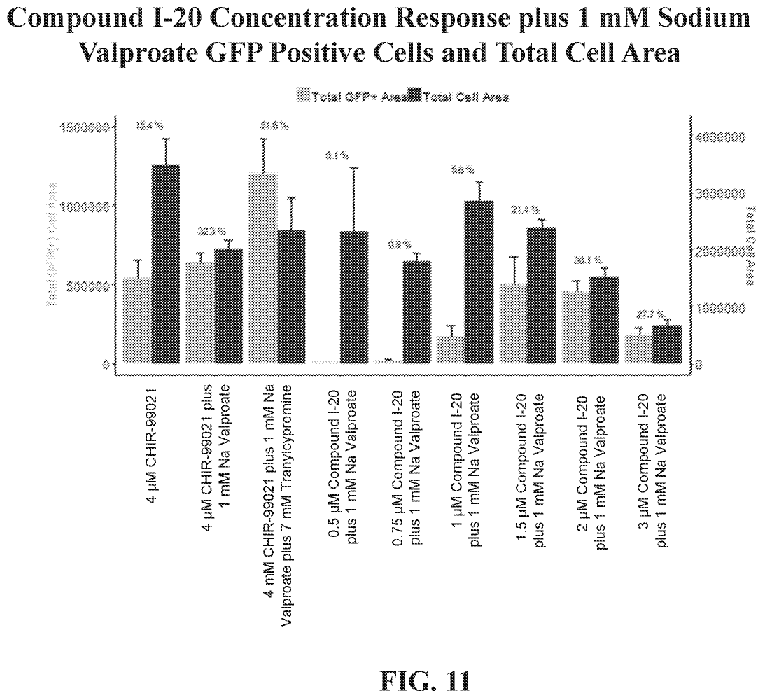

The concentration response for GFP positive cells and total cell area for Compound I-20 at concentrations of 0.5 μM, 0.75 μM, 1 μM, 1.5 μM, 2 μM, and 3 μM in combination with sodium valproate (1 mM) was compared to CHIR-99021 (4 μM) alone, CHIR-99021 (4 μM) in combination with sodium valproate (1 mM), and the combination of CHIR-99021 (4 μM), sodium valproate (1 mM), and tranylcypromine (7 μM). See FIG. 11.

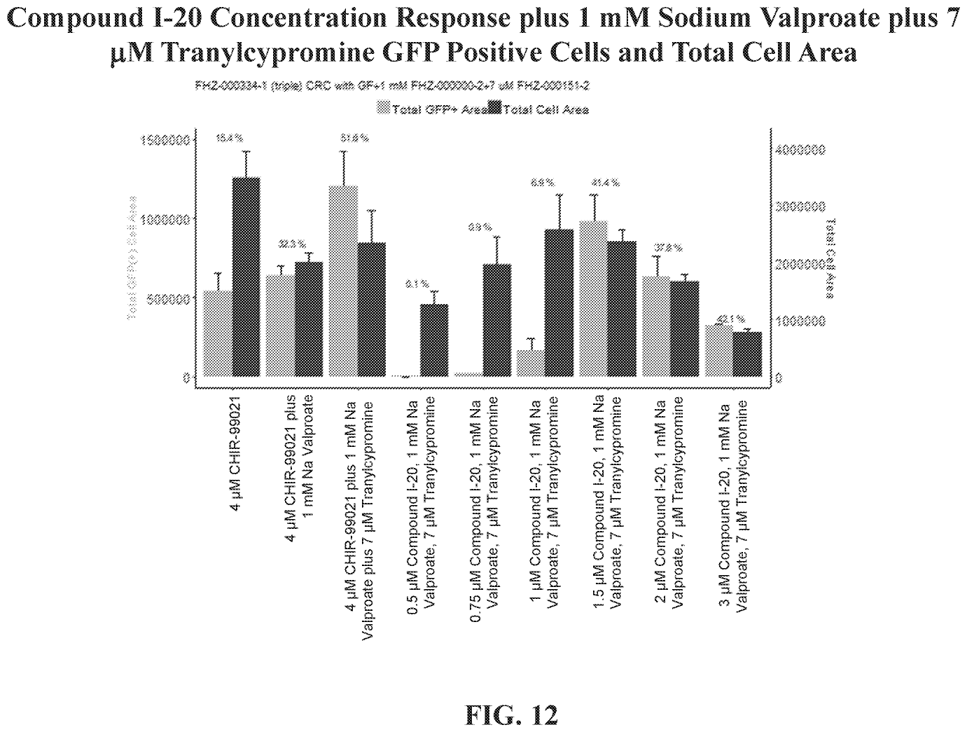

The concentration response for GFP positive cells and total cell area for Compound I-20 at concentrations of 0.5 μM, 0.75 μM, 1 μM, 1.5 μM, 2 μM, and 3 μM in combination with sodium valproate (1 mM) and tranylcypromine (7 μM) was compared to CHIR-99021 (4 μM) alone, CHIR-99021 (4 μM) in combination with sodium valproate (1 mM), and the combination of CHIR-99021 (4 μM), sodium valproate (1 mM), and tranylcypromine (7 μM). See FIG. 12. These data show that Compounds I-7 and I-20 generate Lgr5 GFP+ progenitor cell proliferation in a background of growth factors and generates enrichment of Lgr5 GFP+ cochlear progenitor cells in a background of growth factors.

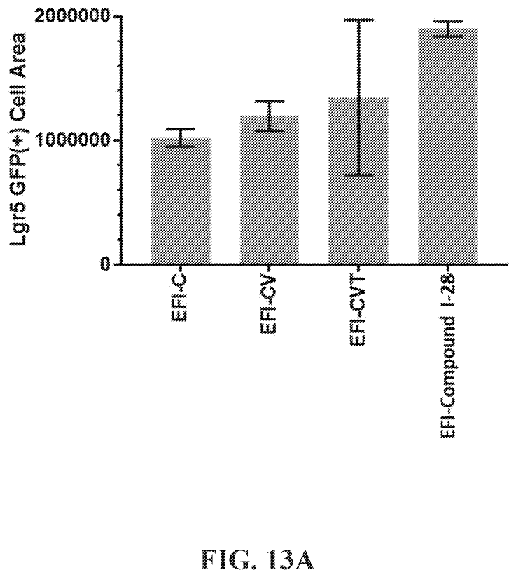

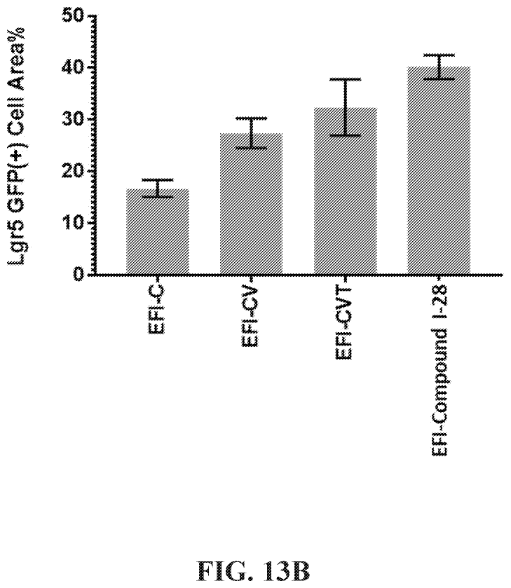

The concentration response Lgr5 positive cell count and total cell count for Compound I-28 (8 μM) in combination with EGF (“E”, 50 ng/mL), bFGF (“F”, 50 ng/mL), and IGR1 (“I”, 50 ng/mL) was compared to CHIR-99021 (“C”, 4 μM) in combination with EGF (“E”, 50 ng/mL), bFGF (“F”, 50 ng/mL), and IGR1 (“I”, 50 ng/mL); CHIR-99021 (“C”, 4 μM) in combination with sodium valproate (“V”, 1 mM), EGF (“E”, 50 ng/mL), bFGF (“F”, 50 ng/mL), and IGR1 (“I”, 50 ng/mL); and CHIR-99021 (“C”, 4 μM) in combination with tranylcypromine (“T”, 7 μM), sodium valproate (“V”, 1 mM), EGF (“E”, 50 ng/mL), bFGF (“F”, 50 ng/mL), and IGR1 (“I”, 50 ng/mL). See FIG. 13A. These data demonstrate that Compound I-28 generates Lgr5 GFP+ progenitor cell proliferation in a background of growth factors.

The concentration response percent of Lgr5 positive cells for Compound I-28 (8 μM) in combination with EGF (“E”, 50 ng/mL), bFGF (“F”, 50 ng/mL), and IGR1 (“I”, 50 ng/mL) was compared to CHIR-99021 (“C”, 4 μM) in combination with EGF (“E”, 50 ng/mL), bFGF (“F”, 50 ng/mL), and IGR1 (“I”, 50 ng/mL); CHIR-99021 (“C”, 4 μM) in combination with sodium valproate (“V”, 1 mM), EGF (“E”, 50 ng/mL), bFGF (“F”, 50 ng/mL), and IGR1 (“I”, 50 ng/mL); and CHIR-99021 (“C”, 4 μM) in combination with tranylcypromine (“T”, 7 μM), sodium valproate (“V”, 1 mM), EGF (“E”, 50 ng/mL), bFGF (“F”, 50 ng/mL), and IGR1 (“I”, 50 ng/mL). See FIG. 13B. These data show that Compound I-28 generates enrichment of Lgr5 GFP+ cochlear progenitor cells in a background of growth factors.

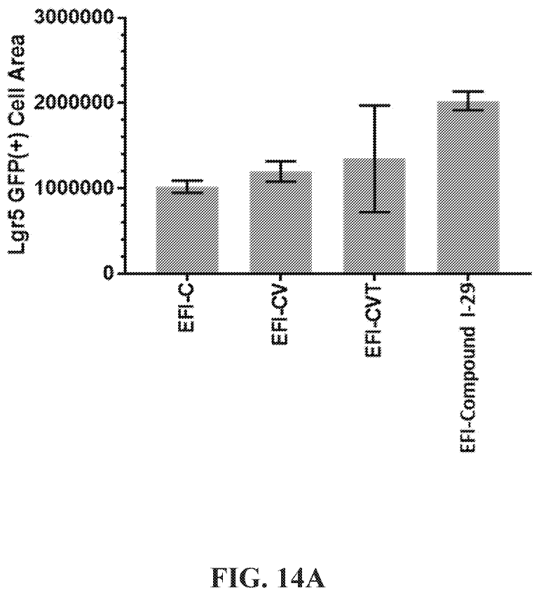

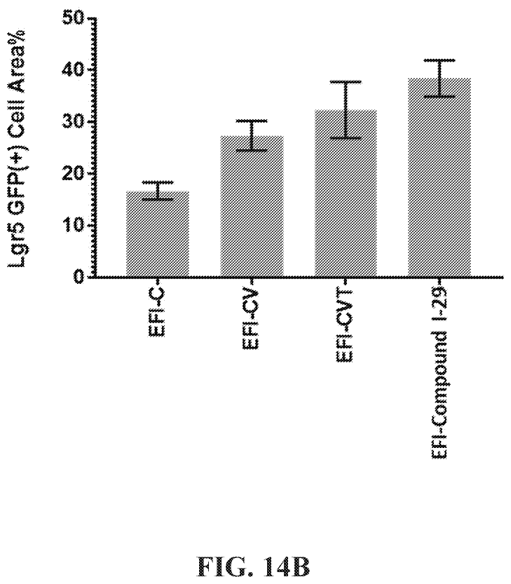

The concentration response Lgr5 positive cell count and total cell count for Compound I-29 (8 μM) in combination with EGF (“E”, 50 ng/mL), bFGF (“F”, 50 ng/mL), and IGR1 (“I”, 50 ng/mL) was compared to CHIR-99021 (“C”, 4 μM) in combination with EGF (“E”, 50 ng/mL), bFGF (“F”, 50 ng/mL), and IGR1 (“I”, 50 ng/mL); CHIR-99021 (“C”, 4 μM) in combination with sodium valproate (“V”, 1 mM), EGF (“E”, 50 ng/mL), bFGF (“F”, 50 ng/mL), and IGR1 (“T”, 50 ng/mL); and CHIR-99021 (“C”, 4 μM) in combination with tranylcypromine (“T”, 7 μM), sodium valproate (“V”, 1 mM), EGF (“E”, 50 ng/mL), bFGF (“F”, 50 ng/mL), and IGR1 (“I”, 50 ng/mL). See FIG. 14A. These data show that Compound I-29 generates Lgr5 GFP+ progenitor cell proliferation in a background of growth factors.

The concentration response percent of Lgr5 positive cells for Compound I-29 (8 μM) in combination with EGF (“E”, 50 ng/mL), bFGF (“F”, 50 ng/mL), and IGR1 (“I”, 50 ng/mL) was compared to CHIR-99021 (“C”, 4 μM) in combination with EGF (“E”, 50 ng/mL), bFGF (“F”, 50 ng/mL), and IGR1 (“I”, 50 ng/mL); CHIR-99021 (“C”, 4 μM) in combination with sodium valproate (“V”, 1 mM), EGF (“E”, 50 ng/mL), bFGF (“F”, 50 ng/mL), and IGR1 (“I”, 50 ng/mL); and CHIR-99021 (“C”, 4 μM) in combination with tranylcypromine (“I”, 7 μM), sodium valproate (“V”, 1 mM), EGF (“E”, 50 ng/mL), bFGF (“F”, 50 ng/mL), and IGR1 (“I”, 50 ng/mL). See FIG. 14B. These data show that Compound I-29 generates enrichment of Lgr5 GFP+ cochlear progenitor cells in a background of growth factors.

Assay: Caco-2

A Caco-2 cell permeability assay can be used for in vitro prediction of drug permeability and absorption, an important feature for a compound to be useful for methods described herein. The apparent permeability (Papp) of the compounds of the present disclosure were determined in Caco-2 cells to measure the rate of drug transport per surface area of the cell membrane. Further the efflux ratios of the compounds of the present disclosure were measured in Caco-2 cells to determine the apparent permeability coefficient in basolateral to apical direction with respect to the apparent permeability coefficient in apical to basolateral direction.

Preparation of Caco-2 Cells

50 μL and 25 mL of cell culture medium were added to each well of the Transwell insert and reservoir, respectively. The HTS transwell plates were incubated at 37° C., 5% CO₂ for 1 hour before cell seeding. Caco-2 cells were diluted to 6.86×10⁵ cells/mL with culture medium and 50 μL of cell suspension were dispensed into the filter well of the 96-well HTS Transwell plate. Cells were cultivated for 14-18 days in a cell culture incubator at 37° C., 5% CO₂95% relative humidity. Cell culture medium was replaced every other day, beginning no later than 24 hours after initial plating.

Assessment of Cell Monolayer Integrity

Medium was removed from the reservoir and each Transwell insert and replaced with prewarmed fresh culture medium. Transepithelial electrical resistance (TEER) across the monolayer was measured using Millicell Epithelial Volt-Ohm measuring system (Millipore, USA). The Plate was returned to the incubator once the measurement was done. The TEER value was calculated according to the following equation: TEER measurement (ohms)×Area of membrane (cm²)=TEER value (ohm·cm²).

Preparation of Solutions

To prepare the HBSS (25 mM HEPES, pH 7.4), accurately weigh 5.958 g of HEPES and 0.35 g sodium hydrogen carbonate and add into 900 mL of pure water, then sonicate to dissolve the content. Transfer 100 mL of 10× HBSS into the solution, and place the solution on a stirrer, slowly adjust pH with sodium hydrate to 7.4, following with filtering. Add 6 μL of stock solution (10 mM) of test compound or control compound to 24 μL of DMSO into the same well to obtain 2 mM stock solutions. Transfer 3 μL of 2 mM solution into 597 μL of transport buffer in one 96 well plate to prepare the 10 μM compound working solution. Shake the plate at 1000 rpm for 10 min. The final concentration of DMSO in the incubation system was 0.5%. Metoprolol, erythromycin, and cimetidine were used as control compounds.

Performing the Drug Transport Assay

Assays can be conducted using Caco-2 cells to assay transport of a compound or composition For example, Caco-2 plates prepared as described above were removed from the incubator. Monolayers were washed twice with pre-warmed HBSS (25 mM HEPES, pH 7.4), then the plates were incubated at 37° C. for 30 minutes. To determine the rate of drug transport in the apical to basolateral direction, 108 μL of the working solution was added to the Transwell insert (apical compartment), and 8 μL sample was immediately transferred from the apical compartment to 72 μL transport buffer and 240 μL of acetonitrile containing IS (100 nM alprazolam, 200 nM caffeine and 100 nM tolbutamide) in a new 96-well plate as the initial donor sample (A-B). The samples were then vortexed at 1000 rpm for 10 minutes. The wells in the receiver plate (basolateral compartment) were filled with 300 μL of transport buffer. To determine the rate of drug transport in the basolateral to apical direction, 308 μL of the working solution were added to the receiver plate wells (basolateral compartment), and 8 μL sample was immediately transferred from the basolateral compartment to 72 μL transport buffer and 240 μL of acetonitrile containing IS (100 nM alprazolam, 200 nM caffeine and 100 nM tolbutamide) into a new 96-well plate as the initial donor sample (B-A), then Vortexed at 1000 rpm for 10 minutes. The Transwell insert (apical compartment) was filled with 100 μL of transport buffer. The apical to basolateral direction and the basolateral to apical direction need to be done at the same time. The multiwell insert plate was placed into the basolateral receiver plate, placed into the incubator and is incubated at 37° C. for 2 hours. At the end of the transport period, 8 μL of samples was transferred from donor sides (apical compartment for Ap→Bl flux, and basolateral compartment for Bl→Ap flux) to 72 μL transport buffer and 240 μL quenching solvents in a new 96-well plate. 80 μL of samples was directly removed from receiver sides (basolateral compartment for Ap→Bl flux, and apical compartment for Bl→Ap flux) and transfer to new 96-well plates with 240 μL of acetonitrile containing IS (100 nM alprazolam, 200 nM caffeine and 100 nM tolbutamide), then vortexed for 10 minutes at 1000 rpm. Samples were then centrifuged at 4,000 rpm for 30 minutes. 100 μL of the supernatant was transferred to a new 96-well plate ensuring the pellet was not disturbed then 100 μL of pure water was added to all samples for analysis by LC-MS/MS. All incubations were performed in duplicate. To determine the Lucifer Yellow leakage after 2 hour transport period, Lucifer yellow working solutions were prepared by diluting the stock solution with HBSS (25 mM HEPES, pH 7.4) to reach the final concentration of 100 μM, then 100 μL of the Lucifer yellow solution was added to the Transwell insert (apical compartment). The wells in the receiver plate (basolateral compartment) were filled with 300 μL of HBSS (25 mM HEPES, pH 7.4), then incubated at 37° C. for 30 minutes. Next, 80 μL was directly removed from the basolateral wells and transferred to new 96 wells plates according to a plate map. Lucifer Yellow fluorescence (to monitor monolayer integrity) was detected using a fluorescence plate reader at 485 nM excitation and 530 nM emission.

Data Analysis

Lucifer yellow leakage of monolayer can be calculated using the following equation.

$$LY Leakage = ( \frac{{I}_{acceptor} \times 0.3}{{I}_{acceptor} \times 0.3 + {I}_{donor} \times 0.1} ) \times 100 \%$$

Where I_{acceptor} is the fluorescence intensity in the acceptor well (0.3 mL), and Idonor is the fluorescence intensity in the donor well (0.1 mL) and expressed as % leakage. Lucifer yellow percentage amount transported values should be less than 1.5%. However, if the Lucifer yellow percentage amount transported value for a particular transwell is higher than 1.5 but the determined digoxin Papp in that transwell is qualitatively similar to that determined in the replicate transwells then, based upon the scientific judgement of the responsible scientist, the monolayer is considered acceptable.

Apparent permeability (Pₐₚₚ) can be calculated for drug transport assays using the following equation:

$${P}_{app} = \frac{dQ / dt}{A \times {D}_{0}}$$

Where: Papp is apparent permeability (cm/s×10⁻⁶); dQ/dt is the rate of drug transport (pmol/second); A is the surface of the membrane (cm²); D₀ is the initial donor concentration (nM; pmol/cm³).

Efflux ratio can be determined using the following equation:

$$Efflux Ratio = \frac{{P}_{app ( B - A )}}{{P}_{app ( A - B )}}$$

Where P_{app (B-A)} indicates the apparent permeability coefficient in basolateral to apical direction, and P_{app (A-B)} indicates the apparent permeability coefficient in apical to basolateral direction.

Compounds of the present disclosure were synthesized and tested for Caco-2 cell penetration Using methods described herein. Activity of exemplary compounds is shown in Table VII below.

TABLE VII

| | P_{app(A-B)} | Efflux |
|---|---|---|
| Compound | (×10⁻⁶, cm/s) | Ratio |
| I-1 | 31 | 0.47 |
| I-3 | 3.3 | 0.27 |
| I-7 | 13 | 0.32 |
| I-8 | 11 | 3.02 |
| I-9 | 3.4 | 0.56 |
| I-10 | 6.3 | 0.56 |
| I-11 | 5.5 | 0.53 |
| I-12 | 21 | 0.72 |
| I-13 | 5.5 | 0.53 |
| I-14 | 8.6 | 0.58 |
| I-15 | 38 | 0.48 |
| I-16 | 17 | 0.6 |
| I-17 | 23 | 0.45 |
| I-18 | 11 | 0.66 |
| I-19 | 5.9 | 0.38 |
| I-20 | 6.1 | 0.29 |
| I-23 | 1.1 | 0.69 |
| I-26 | 7.9 | 0.85 |
| I-27 | 0.94 | 1.96 |
| I-28 | 3.1 | 0.48 |
| I-29 | 9.1 | 0.39 |
| I-32 | 4.2 | 0.47 |
| I-38 | 1.6 | 0.75 |
| I-50 | 9.7 | 1.04 |
| I-57 | 7.1 | 0.4 |
| I-59 | 6.4 | 0.58 |
| I-60 | 0.32 | 0.5 |
| I-61 | 0.11 | 0.85 |
| I-62 | 0.82 | 38 |
| I-66 | 1.8 | 58 |
| I-67 | 2.2 | 0.24 |
| I-68 | 0.06 | 0.63 |
| I-69 | 4.6 | 0.78 |
| I-70 | 2.4 | 1.2 |
| I-81 | 5.9 | 0.59 |
| I-83 | 5.1 | 1.3 |
| I-84 | 4.1 | 2.1 |
| I-86 | 5 | 1.4 |
| I-87 | 4.7 | 1.4 |

*The values shown in the table are approximate values.

These data show that the compounds of the present disclosure are predicted to have cell penetration activity, a desirable feature for their use in a therapeutic composition.

Assay: Therm. Aq Sol (pH 7.4) μM

The aqueous thermodynamic solubilities of the compounds of the present disclosure were measured at pH 7.4.

Procedure for Thermodynamic Solubility Determination

Determining suitability for use of a compound, e.g., as a therapeutic agent can include determining the thermodynamic solubility of the compound. An example of this determination follows: Accurately weigh about 1 mg of the powder of each compound into a glass insert vial and phosphate buffer into each vial of a capless Solubility Sample plate with the volume of 1 mL per mg. Next, add one stir stick to each vial and seal with a molded PTDE/SIL 96-Well Plate Cover. Transfer the Solubility Sample plate to an Eppendorf Thermomixer Comfort plate shaker and shake the plate at 25° C. at 1,100 rpm for 24 hours. At the end of the incubation, open the cover and remove the stir sticks using a big magnet and transfer the samples from the Solubility Sample plate into the filter plate. Using the vacuum manifold, all the samples were filtered. An aliquot of 100 μL was transferred from filtrate to a new 96-well plate by addition of 900 μL of a mixture of H₂O and acetonitrile containing an internal standard (1:1) (10 fold) and 10 μL of the diluted solution was pipetted into a new 96-well plate by addition of 990 μL of a mixture of H₂O and acetonitrile containing internal standard (1:1) (1,000 fold). The dilution factor may be changed according to the solubility value and the LC/MS signal response.

Preparation of Standards (STD)

To prepare standards for the assay, accurately weigh about 1 mg of the powder of each compound into a glass insert vial, add DMSO into each vial of the Standard Plate with the volume of 1 mL per mg to obtain the 1 mg/mL STD sample. Aliquots of 10 μL were taken from 1 mg/mL STD sample to a new 96-well plate followed by addition of 990 μL of a mixture of H₂O and acetonitrile containing internal standard (1:1) to obtain 10 μg/mL STD and then 10 μL aliquots were taken from 10 μg/mL sample to a new 96-well plate followed by addition of 990 μL of a mixture of H₂O and acetonitrile containing internal standard (1:1) to obtain 0.1 μg/mL STD. The concentration of the standard sample may be changed according to the LC/MS signal response.

Data Analysis

The samples were analyzed and quantified against the standard of known concentration using LC/MS/MS. The solubility of the test compound was calculated as follows: [Sample]=Area Ratio sample×DF sample×[STD]/Area Ratio STD DF means the dilution factor. Any value of the compounds that is not within the specified limits was rejected and the experiment was repeated.

Compounds of the present disclosure were synthesized and tested for thermodynamic aqueous solubility at pH 7.4 using methods described above. Activity of exemplary compounds is shown in Table VIII below.

TABLE VIII

| | | Basic |
|---|---|---|
| | Concentration | Cyclopropyl |
| Compound | (μM) | Amine |
| I-7 | <0.12 | |
| I-8 | 35 | |
| I-9 | <1.8 | |
| I-11 | <0.08 | |
| I-12 | 2.7 | |
| I-13 | <1.2 | |
| I-15 | 2.4 | |
| I-16 | 1.1 | |
| I-17 | 0.11 | |
| I-18 | 0.12 | |
| I-19 | <0.03 | |
| I-20 | <0.3 | |
| I-23 | <0.03 | |
| I-27 | 4.7 | Yes |
| I-28 | 4 | Yes |
| I-29 | 1.93 | Yes |
| I-32 | <0.08 | |
| I-38 | <0.01 | |
| I-57 | <0.03 | |
| I-59 | <0.06 | |
| I-60 | <10 | |
| I-61 | <0.002 | |
| I-62 | 54 | Yes |
| I-65 | 0.11 | |
| I-66 | 0.06 | |
| I-67 | 0.23 | |
| I-68 | 0.03 | |
| I-69 | <0.03 | |
| I-70 | 1.6 | Yes |
| I-81 | 1.2 | Yes |
| I-83 | 9.6 | Yes |
| I-84 | 49 | Yes |
| I-86 | 23 | Yes |
| I-87 | 8 | Yes |

*The values shown in the table are approximate values.

These data show that the compounds of the present disclosure, particularly those containing basic cyclopropylamines, demonstrate high aqueous solubility at pH 7.4.

### Equivalents

The details of one or more embodiments of the invention are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the invention to the precise form disclosed, but by the claims appended hereto.

What is claimed is:

1. A compound of Formula (I): 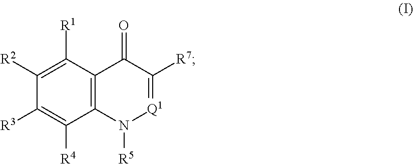
or a pharmaceutically acceptable salt or tautomer thereof, wherein:
Q¹ is CR⁶ or N;
R¹ is selected from the group consisting of H, F, Cl, Br, NO₂, OR^{1a}, SR^{1a}, N(R^{1a})₂, and C₁-C₆ alkyl; wherein each R^{1a} is independently H, C₁-C₆ alkyl, or —C(═O)—(C₁-C₆ alkyl); wherein the C₁-C₆ alkyl is optionally substituted with one or more halogen or phenyl;
R² is selected from the group consisting of H, F, Cl, Br, C₁-C₆ alkyl, and NR¹⁰R¹¹;
R³ is selected from the group consisting of -L-R⁸, C₁-C₈ alkyl, F, N(R^{3a})(R^{3b}), C₁-C₄ alkyl-N(R^{3a})(R^{3b}), OR^{3b}, C₃-C₈ cycloalkyl optionally substituted with N(R^{3a})(R^{3b}), CH₂N(R^{3a})CH₂CN, N(R^{3a})CH₂CN, and aryl optionally substituted with R^{3b}; wherein R^{3a} is H or C₁-C₈ alkyl; R^{3b} is H, C₁-C₈ alkyl, C₂-C₈ alkenyl, C₃-C₈ cycloalkyl, C₄-C₈ heterocycloalkyl, C₄-C₈ cycloalkenyl, phenyl, indanyl, heteroaryl, or —C(═O)—(C₁-C₆ alkyl); wherein the C₁-C₈ alkyl, C₂-C₈ alkenyl, C₃-C₈ cycloalkyl, C₄-C₈ heterocycloalkyl, C₄-C₈ cycloalkenyl, phenyl, heteroaryl, or —C(═O)—(C₁-C₆ alkyl) is optionally substituted with one or more halogen, C₁₋₄ alkyl, OR⁹, NR¹⁰R¹¹, phenyl optionally substituted with C₁-C₄ alkyl, C₃-C₆ cycloalkyl optionally substituted with NR¹⁰R¹¹, or heteroaryl optionally substituted with one or more halogen, C₁-C₄ alkyl, OR⁹, or NR¹⁰R¹¹; wherein when R^{3b} is C₃-C₈ cycloalkyl substituted with at least two C₁-C₄ alkyl substituents, the two C₁-C₄ alkyl substituents together with the carbon atom(s) to which they are attached can form a C₃-C₈ cycloalkyl or C₆-C₁₆ aryl; or wherein R^{3a} and R^{3b} are taken together with the N to which they are attached to form a 3-6 membered heterocycl optionally comprising one or more additional heteroatom selected from N, O and S; that is optionally substituted with one or more OR⁹, NR¹⁰R¹¹, halogen, or C₁-C₄ alkyl;
R⁴ is selected from the group consisting of R^{4a}, F, Cl, Br, OR^{4a}, and N(R^{4a})₂; wherein each R^{4a} is independently H, C₁-C₆ alkyl, C₃-C₆ cycloalkyl, or —C(═O)—(C₁-C₆ alkyl);
R⁵ is selected from the group consisting of H, C₁-C₈ alkyl, C₃-C₈ cycloalkyl, C₄-C₈ cycloalkenyl, C₂-C₈ alkenyl, C₂-C₈ alkynyl, and —C(═O)—(C₁-C₄ alkyl); wherein the C₁-C₈ alkyl, C₃-C₈ cycloalkyl, C₄-C₈cycloalkenyl, C₂-C₈ alkenyl, or C₂-C₈ alkynyl is optionally substituted with one or more fluoro, phenyl, or OR^{1a}; or wherein R^{4a} and R⁵ are taken together with the atoms to which they are attached to form a 5-8 membered heterocycl optionally substituted with halogen, N(R^{3a})(R^{3b}), OR^{1a}, or optionally substituted C₁-C₃ alkyl; wherein when a carbon atom of the heterocycl has two C₁-C₃ alkyl substituents attached thereto, the two C₁-C₃ alkyl substituents together with the carbon atom to which they are attached can form a 3-8 membered cycloalkyl or heterocycl comprising one or more heteroatoms selected from O, N and S;
R⁶ is selected from the group consisting of H, F, Cl, Br, and C₁-C₆ alkyl;
R⁷ is selected from the group consisting of CN, tetrazolyl, CH₂OR^{7a}, CFHOR^{7a}, CF₂OR^{7a}, CO₂R^{7a}, CON(R^{7b})₂, and C(═NH)—N(R^{7b})₂; wherein R^{7a} is H, C₁-C₆ alkyl or C₃-C₆ cycloalkyl; and each R^{7b} is independently H, OH, C₁-C₆ alkyl, or C₃-C₆ cycloalkyl;
L is selected from the group consisting of a bond, —(CH₂)₁₋₄—, —C₄-C₈cycloalkenyl-, —(CH₂)ₙN(R^{La})(CH₂)ₙ—, -cycloalkyl-N(R^{La})—, —(CH₂)ₙO—, -aryl-, -heterocycl-, and -heteroaryl-; wherein L is optionally substituted with one or more halo or C₁-C₄ alkyl; wherein Ru is H or C₁-C₈ alkyl; and each n is independently 0 to 4;
R⁸ is selected from the group consisting of C₃-C₈ cycloalkyl-N(R^{8a})(R^{8b}), aryl-C₃-C₈ cycloalkyl-N(R^{8a})(R^{8b}), N(R^{8a})—C₃-C₈ cycloalkyl-aryl, C₄-C₈ cycloalkenyl, OR^{8b}, and N(R^{8a})(R^{8b}); wherein R⁸ is optionally substituted with F or C₁-C₆ alkyl; R^{8a} is H or C₁-C₈ alkyl; and R^{8b} is H or C₁-C₈ alkyl;
R⁹ is H, C₁-C₆ alkyl, or —C(═O)—(C₁-C₆ alkyl); and
R¹⁰ and R¹¹ are each independently selected from H, C₁-C₈ alkyl, C₂-C₈ alkenyl, C₃-C₈ cycloalkyl, C₄-C₈ cycloalkenyl, or —C(═O)—(C₁-C₆ alkyl), wherein the C₁-C₈ alkyl, C₂-C₈ alkenyl, C₃-C₈ cycloalkyl, or C₄-C₈ cycloalkenyl is optionally substituted with one or more F, C₁-C₄ alkyl, optionally substituted phenyl, optionally substituted heteroaryl, or indanyl.

2. A compound of Formula (II): 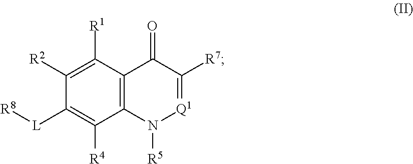
or a pharmaceutically acceptable salt or tautomer thereof, wherein:
Q¹ is CR⁶ or N;
R¹ is selected from the group consisting of H, F, Cl, Br, NO₂, OR^{1a}, SR^{1a}, N(R^{1a})₂, and C₁-C₆ alkyl; wherein each R^{1a} is independently H, C₁-C₆ alkyl, or —C(═O)—(C₁-C₆ alkyl); wherein the C₁-C₆ alkyl is optionally substituted with one or more halogen or phenyl;
R² is selected from the group consisting of H, F, Cl, Br, C₁-C₆ alkyl, and NR¹⁰R¹¹;
R⁴ is selected from the group consisting of R^{4a}, F, Cl, Br, OR^{4a}, and N(R^{4a})₂; wherein each R^{4a} is independently H, C₁-C₆ alkyl, C₃-C₆ cycloalkyl, or —C(═O)—(C₁-C₆ alkyl);
R⁵ is selected from the group consisting of H, C₁-C₈ alkyl, C₃-C₈ cycloalkyl, C₄-C₈ cycloalkenyl, C₂-C₈ alkenyl, C₂-C₈ alkynyl, and —C(═O)—(C₁-C₄ alkyl); wherein the C₁-C₈ alkyl, C₃-C₈ cycloalkyl, C₄-C₈ cycloalkenyl, C₂-C₈ alkenyl, or C₂-C₈ alkynyl is optionally substituted with one or more fluoro, phenyl, or OR^{1a}; or wherein R^{4a} and R⁵ are taken together with the atoms to which they are attached to form a 5-8 membered heterocycl optionally substituted with halogen, N(R^{3a})(R^{3b}), OR^{1a}, or optionally substituted C₁-C₃ alkyl; wherein when a carbon atom of the heterocycl has two C₁-C₃ alkyl substituents attached thereto, the two C₁-C₃ alkyl substituents together with the carbon atom to which they are attached can form a 3-8 membered cycloalkyl or heterocycl comprising one or more heteroatoms selected from O, N and S;
R⁶ is selected from the group consisting of H, F, Cl, Br, and C₁-C₆ alkyl;
R⁷ is selected from the group consisting of CN, tetrazolyl, CH₂OR^{7a}, CFHOR^{7a}, CF₂OR^{7a}, CO₂R^{7a}, CON(R^{7b})₂, and C(═NH)—N(R^{7b})₂; wherein R^{7a} is H, C₁-C₆ alkyl or C₃-C₆ cycloalkyl; and each R^{7b} is independently H, OH, C₁-C₆ alkyl, or C₃-C₆ cycloalkyl;
L is selected from the group consisting of a bond, —(CH₂)₁₋₄—, —C₄-C₈ cycloalkenyl-, —(CH₂)ₙN(R^{La})(CH₂)ₙ—, -cycloalkyl-N(R^{La})—, —(CH₂)ₙO—, -aryl-, -heterocycl-, and -heteroaryl-; wherein L is optionally substituted with one or more halo or C₁-C₄ alkyl; wherein R^{La} is H or C₁-C₈ alkyl; and each n is independently 0 to 4;
R⁸ is selected from the group consisting of C₃-C₈ cycloalkyl-N(R^{8a})(R^{8b}), aryl-C₃-C₈ cycloalkyl-N(R^{8a})(R^{8b}), N(R^{8a})—C₃-C₈ cycloalkyl-aryl, C₄-C₈ cycloalkenyl, OR^{8b}, and N(R^{8a})(R^{8b}); wherein R⁸ is optionally substituted with F or C₁-C₆ alkyl; R^{8a} is H or C₁-C₈ alkyl; and R^{8b} is H or C₁-C₈ alkyl.

3. The compound of claim 1 or claim 2, wherein Q¹ is N.

4. The compound of claim 1 or claim 2, wherein Q¹ is CR⁶.

5. The compound of any one of the preceding claims, wherein R¹ is selected from the group consisting of H, F, NO₂, OH, N(R^{1a})₂, and C₁-C₃ alkyl.

6. The compound of any one of the preceding claims, wherein R¹ is selected from the group consisting of H, NH₂, NHCH₂Ph, NO₂, and CH₃.

7. The compound of any one of the preceding claims, wherein R¹ is NH₂.

8. The compound of any one of the preceding claims, wherein R² is F.

9. The compound of any one of the preceding claims, wherein R² is NHCH₂CH₃.

10. The compound of any one of the preceding claims, wherein R³ is selected from the group consisting of N(R^{3a})(R^{3b}), C₁₋₃ alkyl-N(R^{3a})(R^{3b}), OR^{3b}, CH₂N(R^{3a})CH₂CN, N(R^{3a})CH₂CN, aryl optionally substituted with R^{3b}, and C₃-C₈ cycloalkyl optionally substituted with NR¹⁰R¹¹.

11. The compound of any one of the preceding claims, wherein R³ is N(R^{3a})(R^{3b}).

12. The compound of any one of the preceding claims, wherein R³ is NH(R^{3b}).

13. The compound of any one of the preceding claims, wherein R³ is NH(R^{3b}) and R^{3b} is selected from the group consisting of C₃-C₈ cycloalkyl, C₄-C₈ heterocycloalkyl, C₃-C₈ cycloalkyl substituted with NR¹⁰R¹¹, C₃-C₈ cycloalkyl substituted with heteroaryl, C₁-C₈ alkyl substituted with phenyl, C₁-C₈ alkyl substituted with NR¹⁰R¹¹, indanyl, and phenyl optionally substituted with C₃-C₆ cycloalkyl optionally substituted with NR¹⁰R¹¹.

14. The compound of any one of the preceding claims, wherein R³ is NH(R^{3b}) and R^{3b} is selected from the group consisting of C₃-C₈ cycloalkyl substituted with NHR¹¹, C₁-C₈ alkyl substituted with NHR¹¹, and phenyl optionally substituted with C₃-C₆ cycloalkyl optionally substituted with NHR¹¹, wherein R¹¹ is selected from the group consisting of H and C₃-C₈ cycloalkyl substituted with an optionally substituted phenyl.

15. The compound of any one of the preceding claims, wherein R³ is NH(R^{3b}) and R^{3b} is C₃-C₈ cycloalkyl or C₄-C₈ heterocycloalkyl.

16. The compound of any one of the preceding claims, R³ is selected from the group consisting of

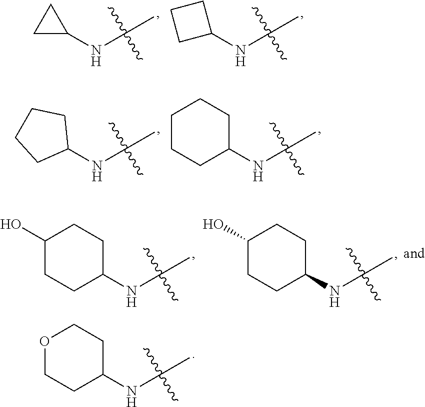

17. The compound of any one of the preceding claims, wherein R³ is N(R^{3a})(R^{3b}) and R^{3b} is C₃-C₈ cycloalkyl optionally substituted with an optionally substituted phenyl.

18. The compound of any one of the preceding claims, wherein R³ is NH(R^{3b}) and R^{3b} is cyclopropyl substituted with phenyl or cyclobutyl substituted with phenyl.

19. The compound of any one of the preceding claims, R³ is selected from the group consisting of

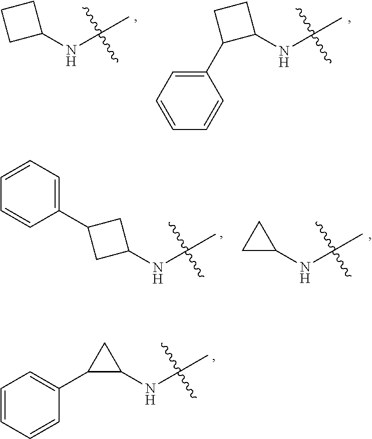 [SMILES: *NC1CCC1 ; *NC1CCC1c1ccccc1 ; *NC1CC(c2ccccc2)C1 ; *NC1CC1 ; *NC1CC1c1ccccc1]

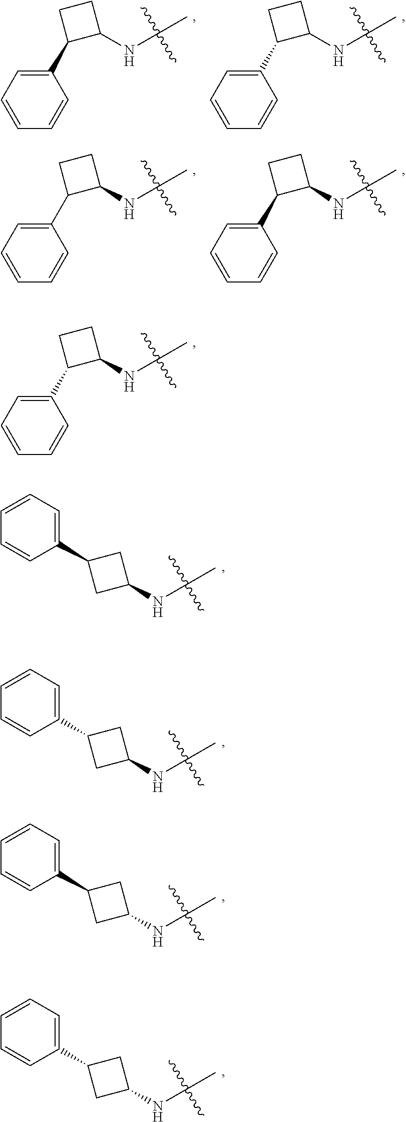

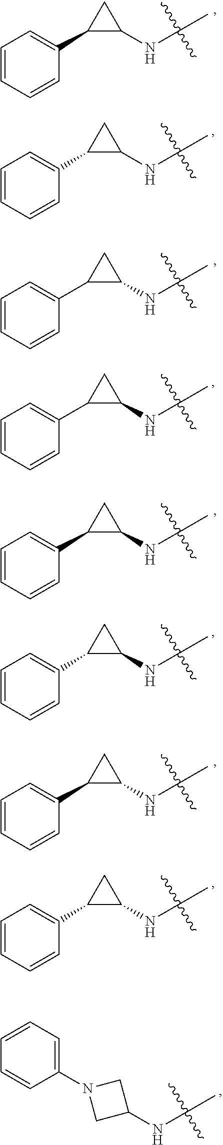

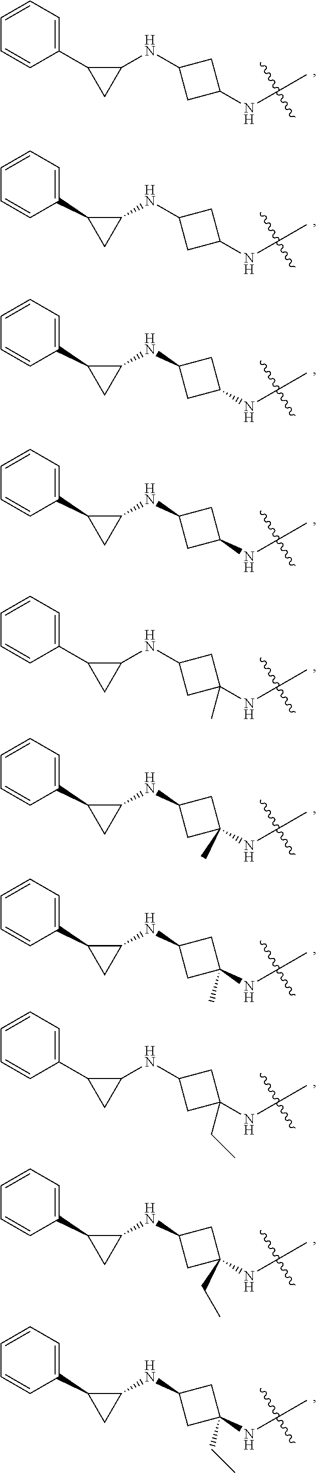

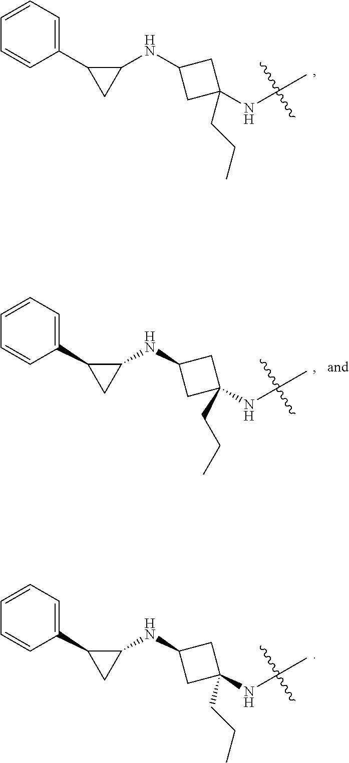 [SMILES: *NC1(CCC)CC(NC2CC2c2ccccc2)C1 ; "," ; *N[C@]1(CCC)C[C@@H](N[C@@H]2C[C@H]2c2ccccc2)C1 ; "," ; *N[C@]1(CCC)C[C@H](N[C@@H]2C[C@H]2c2ccccc2)C1 ; "."]

20. The compound of any one of the preceding claims, wherein R³ is NH(R^{3a})(R^{3b}) and R^{3b} is C₃-C₈ cycloalkyl optionally substituted with one or more C₁-C₄ alkyl and phenyl optionally substituted with C₁-C₄ alkyl.

21. The compound of any one of the preceding claims, wherein R³ is NH(R^{3b}) and R^{3b} is cyclopropyl substituted with one or more methyl and one or more phenyl.

22. The compound of any one of the preceding claims, wherein R³ is NH(R^{3b}) and R^{3b} is C₃-C₈ cycloalkyl substituted with NR¹⁰R¹¹.

23. The compound of any one of the preceding claims, wherein R³ is NH(R^{3b}) and R^{3b} is C₃-C₈ cycloalkyl substituted with NHR¹¹ and R¹¹ is C₃-C₈ cycloalkyl substituted with an optionally substituted phenyl.

24. The compound of any one of the preceding claims, R³ is selected from the group consisting of

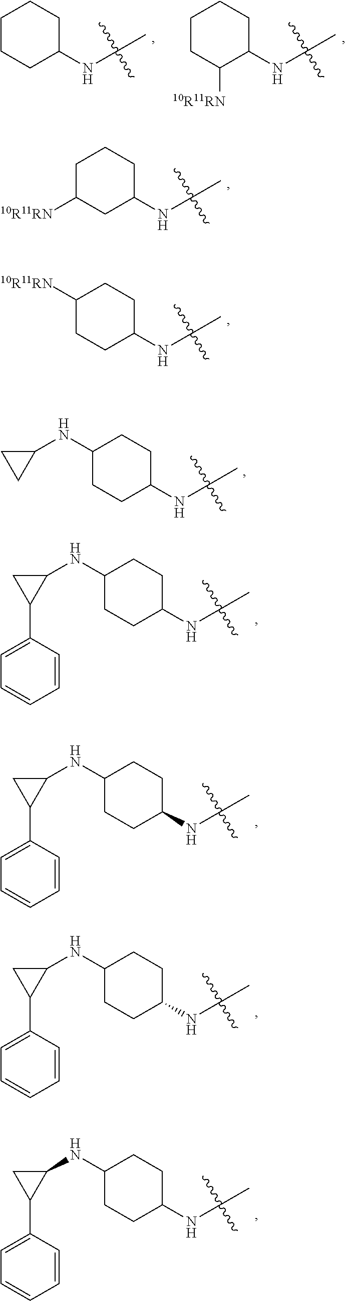

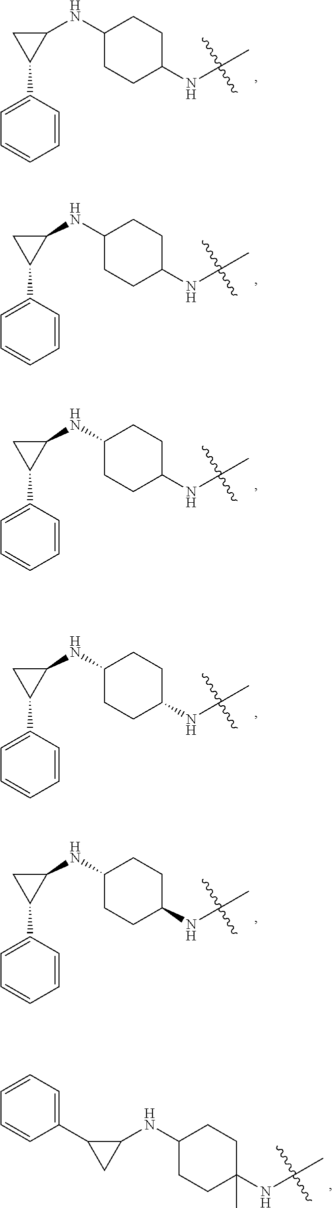

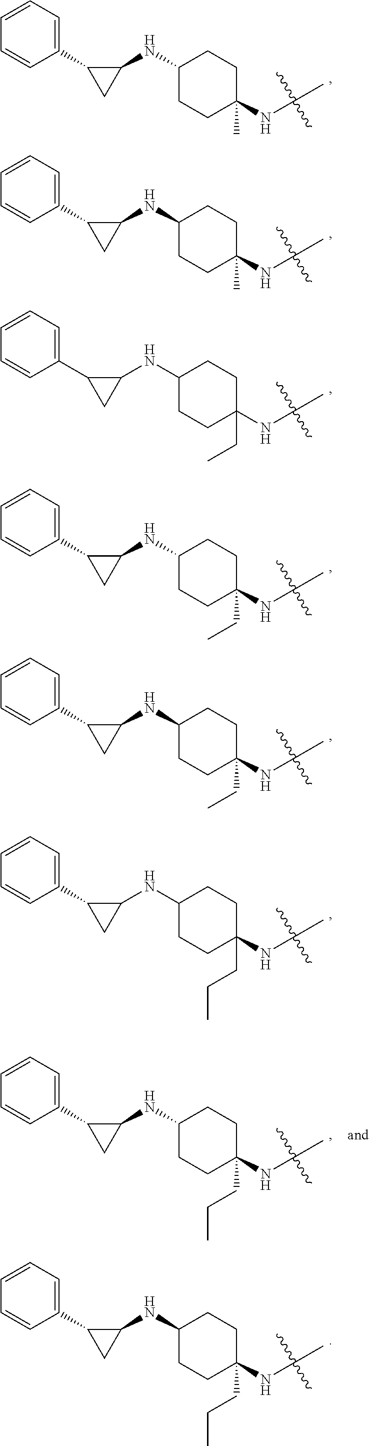

25. The compound of any one of the preceding claims, wherein R³ is NH(R^{3b}) and R^{3b} is C₃-C₈ cycloalkyl substituted with heteroaryl.

26. The compound of any one of the preceding claims wherein R³ is selected from the group consisting of:

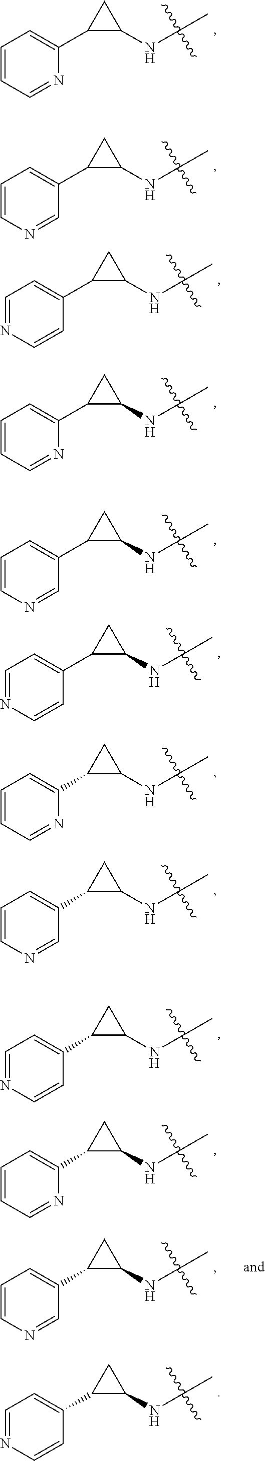

27. The compound of any one of the preceding claims, wherein R³ is NH(R^{3b}) and R^{3b} is C₁-C₈ alkyl substituted with phenyl.

28. The compound of any one of the preceding claims, wherein R³ is selected from the group consisting of:

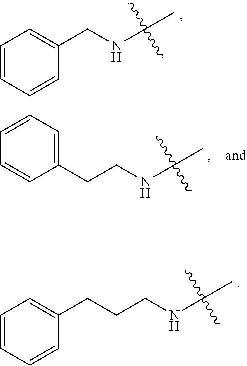

29. The compound of any one of the preceding claims, wherein R³ is NH(R^{3b}) and R^{3b} is C₁-C₈ alkyl substituted with NR¹⁰R¹¹.

30. The compound of any one of the preceding claims, wherein R³ is NH(R^{3b}) and R^{3b} is C₁-C₈ alkyl substituted with NHR¹¹ and R¹¹ is C₃-C₈ cycloalkyl substituted with an optionally substituted phenyl.

31. The compound of any one of the preceding claims, R³ is selected from the group consisting

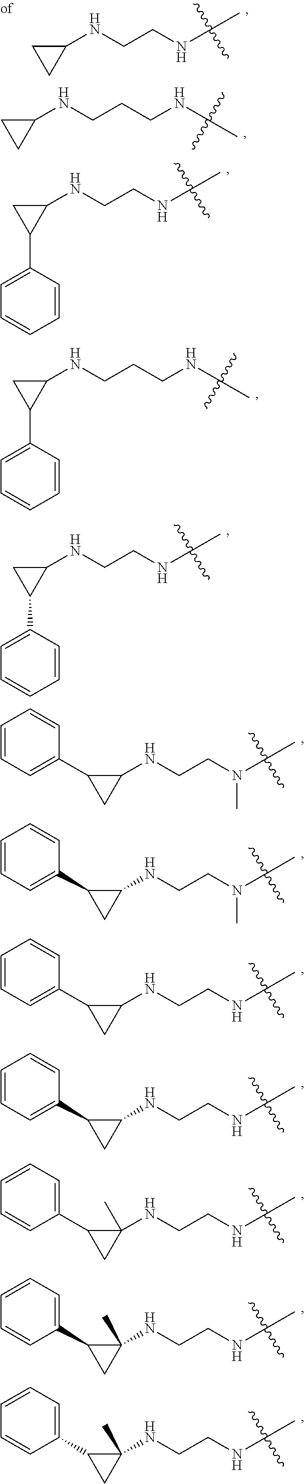

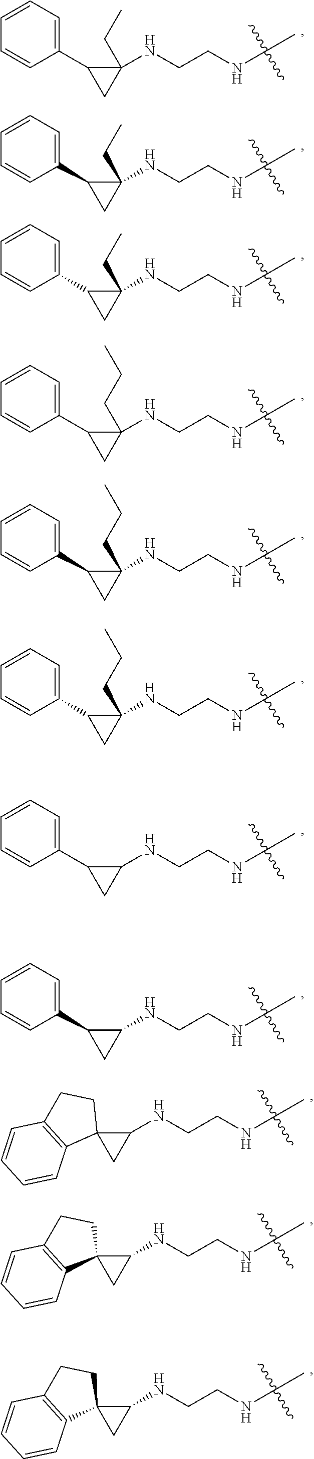

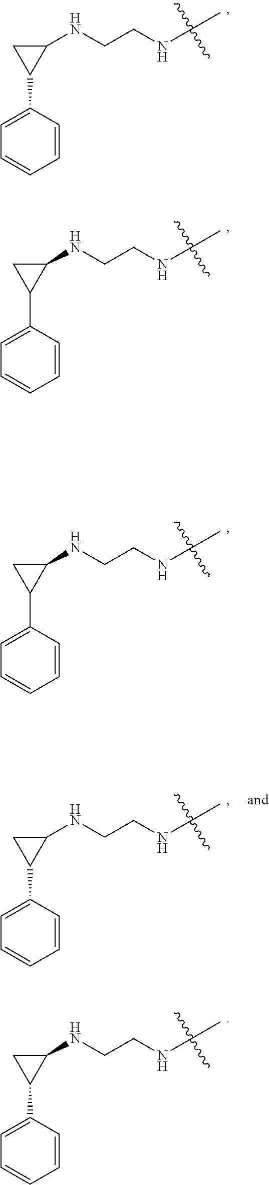

32. The compound of any one of the preceding claims, wherein R³ is NH(R^{3b}) and R^{3b} is indanyl or phenyl, wherein the phenyl is optionally substituted with C₃-C₆ cycloalkyl optionally substituted with NR¹⁰R¹¹.

33. The compound of any one of the preceding claims, R³ is selected from the group consisting of

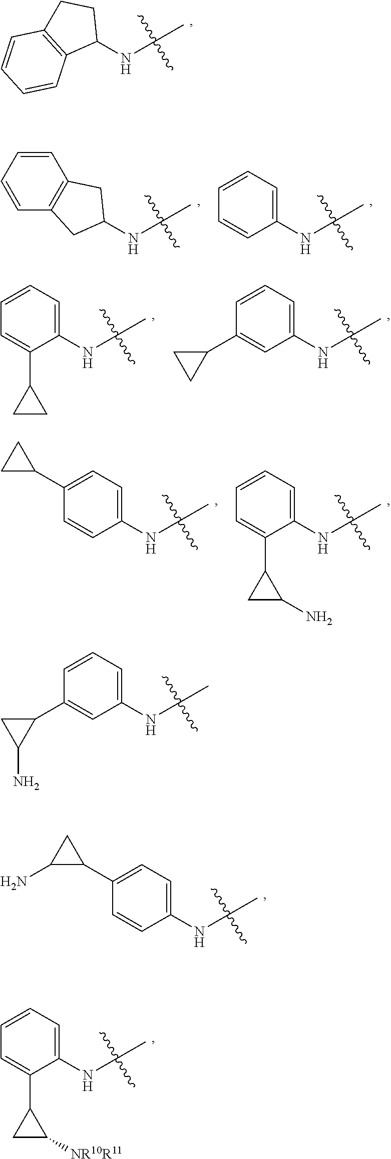

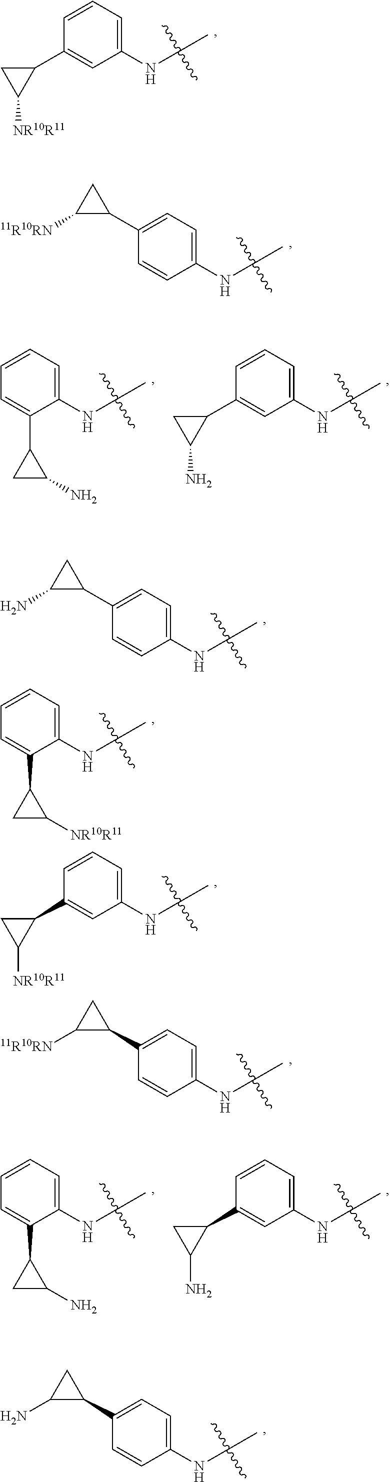

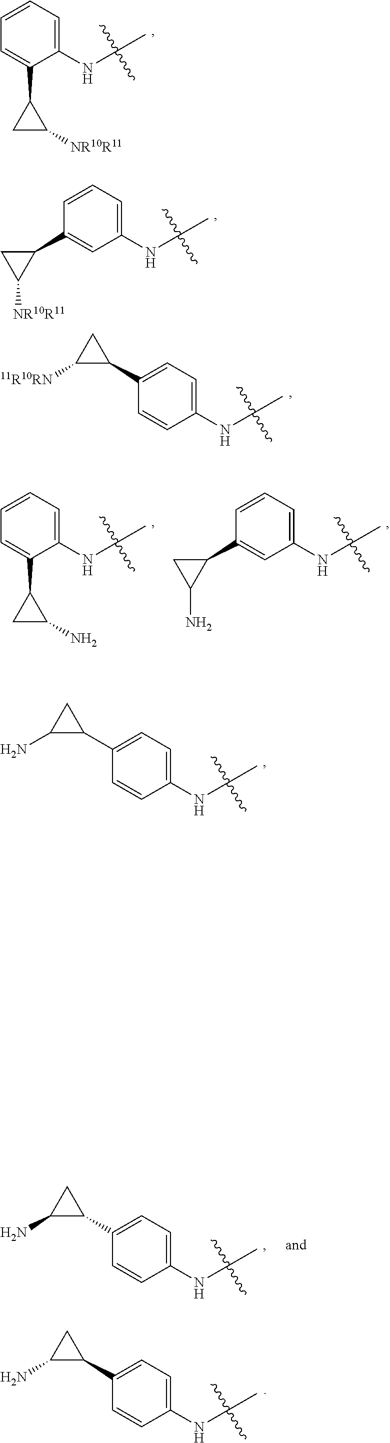

34. The compound of any one of the preceding claims, wherein R³ is N(R^{3a})(R^{3b}) wherein R^{3a} and R^{3b} are taken together with the N to which they are attached to form a 3-6 membered heterocycl substituted with one or more NR¹⁰R¹¹.

35. The compound of any one of the preceding claims, wherein R³ is N(R^{3a})(R^{3b}) wherein R^{3a} and R^{3b} are taken together with the N to which they are attached to form a 6 membered heterocycl substituted with NR¹⁰R¹¹ wherein R¹⁰ is H and R¹¹ is C₃-C₈ cycloalkyl substituted with an optionally substituted phenyl.

36. The compound of any one of the preceding claims, wherein R³ is selected from the group consisting of

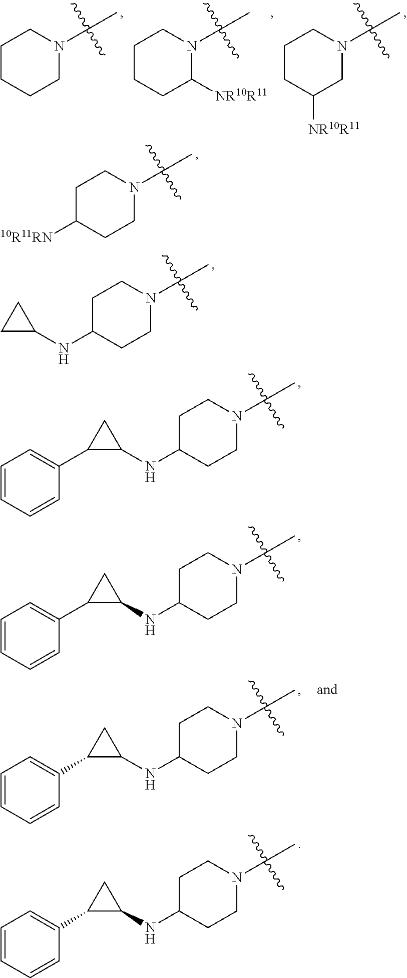

37. The compound of any one of the preceding claims, wherein R³ is NH(R^{3b}) and R^{3b} is C₃-C₈ cycloalkyl substituted with two C₁-C₄ alkyl substitutents and optionally further substituted with phenyl; and wherein the two C₁-C₄ alkyl substituents together with the carbon atom(s) to which they are attached form a C₃-C₈ cycloalkyl or C₆-C₁₆ aryl.

38. The compound of any one of the preceding claims, wherein R³ is elected from the group consisting of:

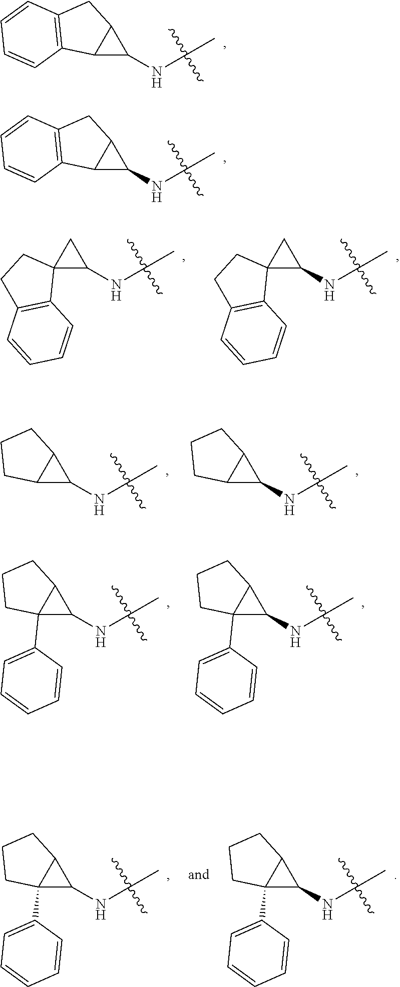

39. The compound of any one of the preceding claims, wherein R³ is C₁-C₄ alkyl-N(R^{3a})(R^{3b}).

40. The compound of any one of the preceding claims, wherein R³ is C₁-C₄ alkyl-NH(R^{3b}) and R^{3b} is C₃-C₈ cycloalkyl substituted with phenyl.

41. The compound of any one of the preceding claims, wherein R³ is selected from the group consisting of:

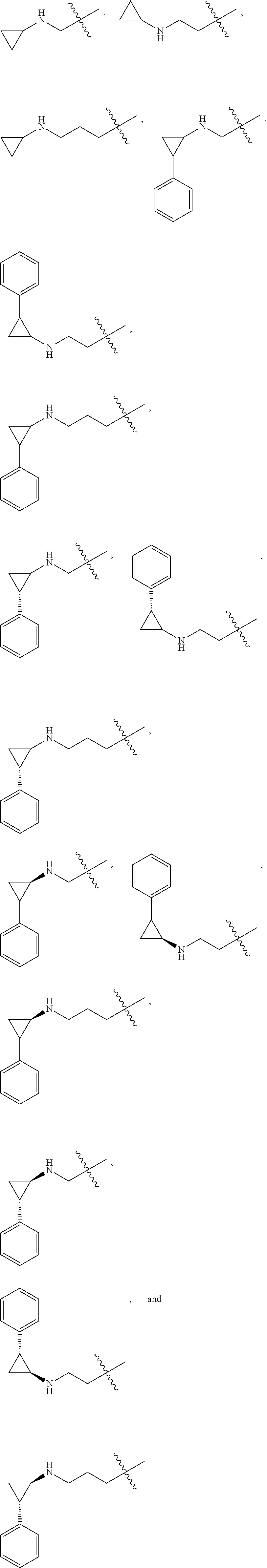

42. The compound of any one of the preceding claims, wherein R³ is OR^{3b}.

43. The compound of any one of the preceding claims, R³ is selected from

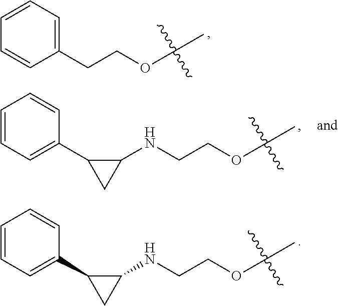

44. The compound of any one of the preceding claims, wherein R³ is selected from the group consisting of CH₂N(R^{3a})CH₂CN and N(R^{3a})CH₂CN.

45. The compound of any one of the preceding claims, wherein R³ is selected from the group consisting of CH₂NHCH₂CN, CH₂N(CH₃)CH₂CN, NHCH₂CN, and N(CH₃)CH₂CN.

46. The compound of any one of the preceding claims, wherein R³ is aryl optionally substituted with R^{3b}.

47. The compound of any one of the preceding claims, wherein R³ is aryl optionally substituted with R^{3b} and R^{3b} is C₃-C₆ cycloalkyl optionally substituted with NR¹⁰R¹¹.

48. The compound of any one of the preceding claims, wherein R³ is selected from the group consisting of:

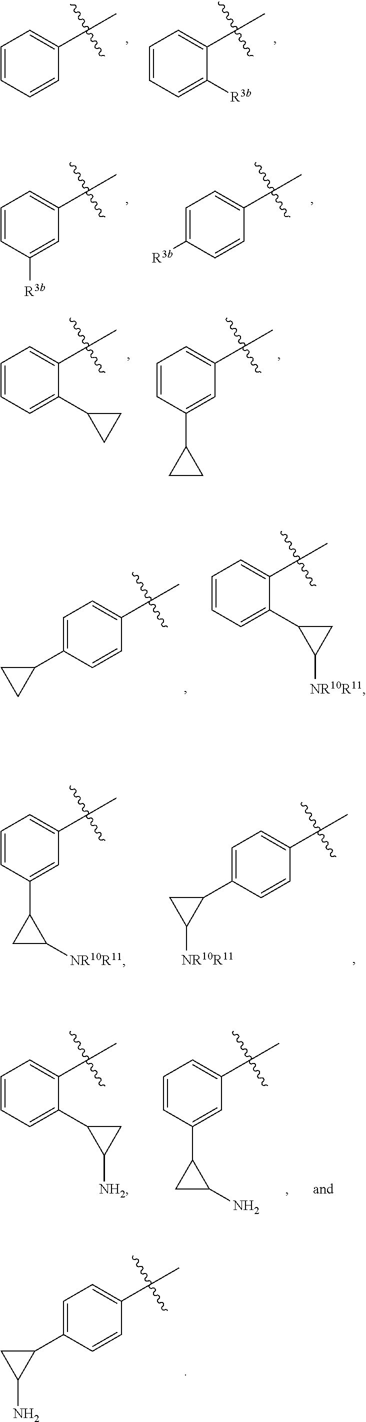

49. The compound of any one of the preceding claims, wherein R³ is C₃-C₈ cycloalkyl optionally substituted with NR¹⁰R¹¹.

50. The compound of any one of the preceding claims, R³ is selected from the group consisting of

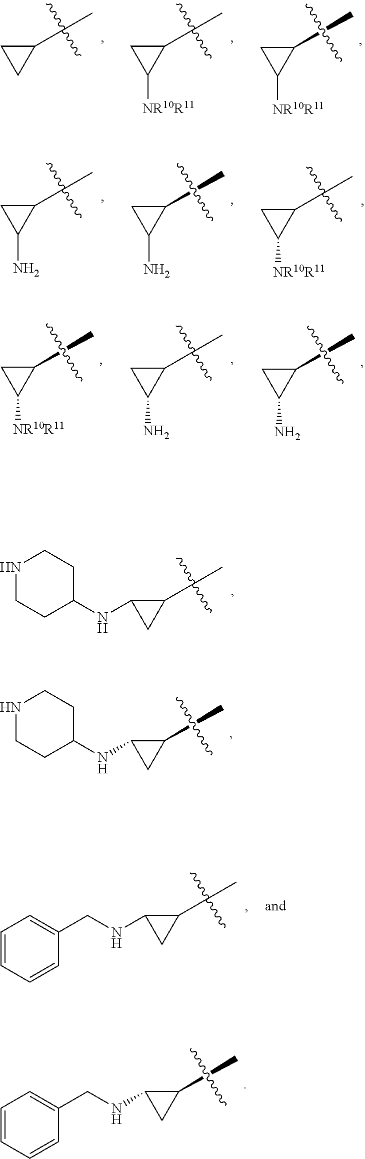

51. The compound of any one of the preceding claims, wherein R³ is N(R^{3a})(R^{3b}).

52. The compound of any one of the preceding claims, wherein N(R^{3a})(R^{3b}) is further defined by: 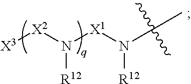
wherein each R¹² is independently selected from the group consisting of H and C₁-C₈ alkyl; wherein X¹ is selected from the group consisting of C₃-C₈ cycloalkyl, C₁-C₈ alkyl, 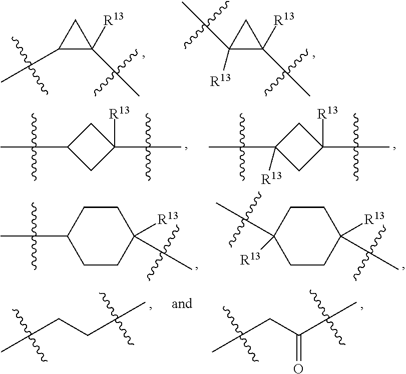
optionally wherein X¹ is substituted with one or more halogen, C₃-C₆ cycloalkyl or C₁₋₄ alkyl; wherein X² is selected from the group consisting of C₃-C₈ cycloalkyl, C₁-C₈ alkyl, 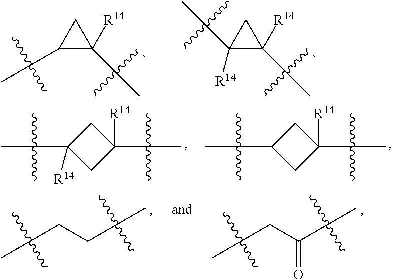
optionally wherein X² is substituted with one or more halogen, C₃-C₆ cycloalkyl or C₁₋₄ alkyl; wherein q is 0 or 1; wherein in X³ is selected from the group consisting of H, phenyl, phenyl substituted with C₁-C₄ alkyl, C₃-C₈ cycloalkyl, and C₁-C₈ alkyl, optionally wherein X³ is substituted with one or more halogen, C₃-C₆ cycloalkyl or C₁₋₄ alkyl; wherein R¹³ is independently selected from the group consisting of H, C₃-C₈ cycloalkyl, and C₁-C₈ alkyl; and wherein R¹⁴ is independently selected from the group consisting of H, C₃-C₈ cycloalkyl, and C₁-C₈ alkyl.

53. The compound of an one of the preceding claims, wherein X¹ is selected from the group consisting of

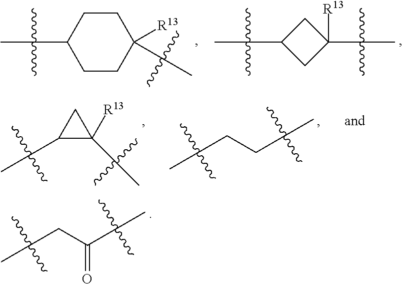

54. The compound of any one of the preceding claims, wherein X² is selected from the group consisting of

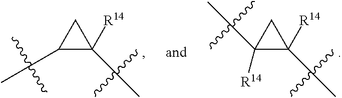

55. The compound of any one of the preceding claims, wherein X³ is selected from the group consisting of H, phenyl, and phenyl substituted with C₁-C₄ alkyl.

56. The compound of any one of the preceding claims, wherein q is 1; X¹ is selected from the group consisting of 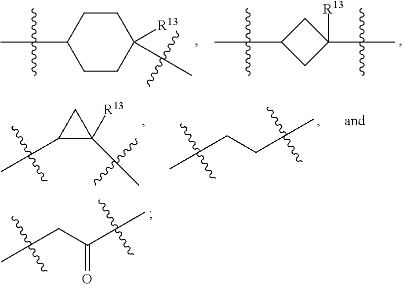 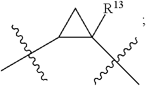
X³ is selected from the group consisting of phenyl and phenyl substituted with C₁-C₄ alkyl; R¹² is H; R¹³ is H or C₁-C₈ alkyl; and R¹⁴ is H or C₁-C₈ alkyl.

57. The compound of any one of the preceding claims, q is 0; X¹ is selected from the group consisting of 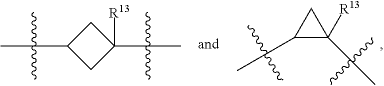
X³ is selected from the group consisting of phenyl and phenyl substituted with C₁-C₄ alkyl; R¹² is H; and R¹³ is H or C₁-C₈ alkyl.

58. The compound of any one of the preceding claims, wherein R¹² is independently selected from the group consisting of H, methyl, ethyl, n-propyl, isopropyl, and n-butyl.

59. The compound of any one of the preceding claims, wherein R¹³ is independently selected from the group consisting of H, methyl, ethyl, n-propyl, isopropyl, and n-butyl.

60. The compound of any one of the preceding claims, wherein R¹⁴ is independently selected from the group consisting of H, methyl, ethyl, n-propyl, isopropyl, and n-butyl.

61. The compound of any one of the preceding claims, wherein q is 1.

62. The compound of any one of the preceding claims, wherein q is 0.

63. The compound of any one of the preceding claims, wherein R⁴ is selected from the group consisting of H, Cl, and F.

64. The compound of any one of the preceding claims, wherein R⁴ is H.

65. The compound of any one of the preceding claims, wherein R⁵ is selected from the group consisting of C₁-C₈ alkyl and C₃-C₈ cycloalkyl.

66. The compound of any one of the preceding claims, wherein R⁵ is selected from the group consisting of CH₃, CH₂CH₃, CH₂CH₂CH₃, CH(CH₃)₂, CH(CH₂CH₃)₂, and cyclopropyl.

67. The compound of any one of the preceding claims, wherein R⁵ is CH₂CH₃.

68. The compound of any one of the preceding claims, wherein R⁵ is CH₂CH₂OH.

69. The compound of any one of the preceding claims, wherein R⁶ is H.

70. The compound of any one of the preceding claims, wherein R⁷ is selected from the group consisting of CN, tetrazolyl, CH₂OR^{7a}, CO₂R^{7a}, CON(R^{7b})₂, and C(═NH)—N(R^{7b})₂.

71. The compound of any one of the preceding claims, wherein R⁷ is selected from the group consisting of CN, CH₂OH, CO₂H, CO₂CH₃, CO₂CH₂CH₃, CONH₂, CONHOH, CONHCH₃, CON(CH3)₂, and C(═NH)—NHOH.

72. The compound of any one of the preceding claims, wherein R⁷ is selected from the group consisting of CN and CO₂H.

73. The compound of any one of the preceding claims, wherein L is selected from the group consisting of a bond, —(CH₂)₁₋₄—, —(CH₂)ₙN(R^{La})(CH₂)ₙ—, and -cycloalkyl-N(R^{La})—.

74. The compound of any one of the preceding claims, wherein L is selected from the group consisting of a bond, —CH₂—, —N(R^{La})CH₂—, —CH₂N(R^{La})—, -cyclopropyl-N(R^{La})—, and -cyclobutyl-N(R^{La})—.

75. The compound of any one of the preceding claims, wherein R⁸ is selected from the group consisting of C₃-C₈ cycloalkyl-N(R^{8a})(R^{8b}), N(R^{8a})—C₃-C₈ cycloalkyl-aryl, and aryl-C₃-C₈ cycloalkyl-N(R^{8a})(R^{8b}).

76. The compound of any one of the preceding claims, wherein R⁸ is selected from the group consisting of cyclopropyl-NH₂, cyclobutyl-NH₂, phenyl-cyclopropyl-NH₂, phenyl-cyclobutyl-NH₂, NH-cyclopropyl-phenyl, and NH-cyclobutyl-phenyl.

77. The compound of any one of the preceding claims, wherein the compound is one of Formulae (Ia), (Ib), (Ic), (Id), (Ie), (If), (Ig), (Ih), (Ii), (Ij), or (Ik): 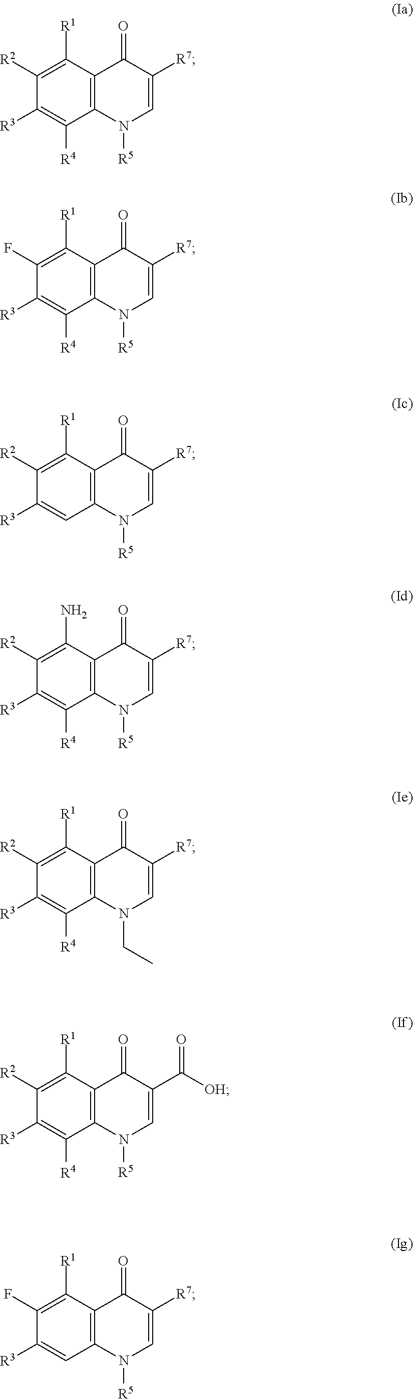 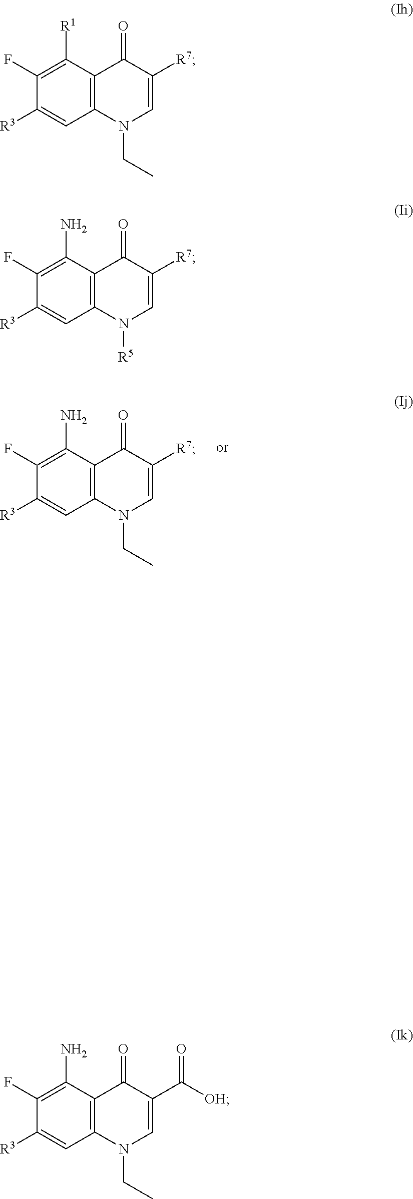
or a pharmaceutically acceptable salt or tautomer thereof, wherein R¹, R², R³, R⁴, R⁵, and R⁷ are as described herein.

78. The compound of any one of the preceding claims, wherein the compound is of Formulae (IIIa), (IIIb), (IIIc), or (IIId): 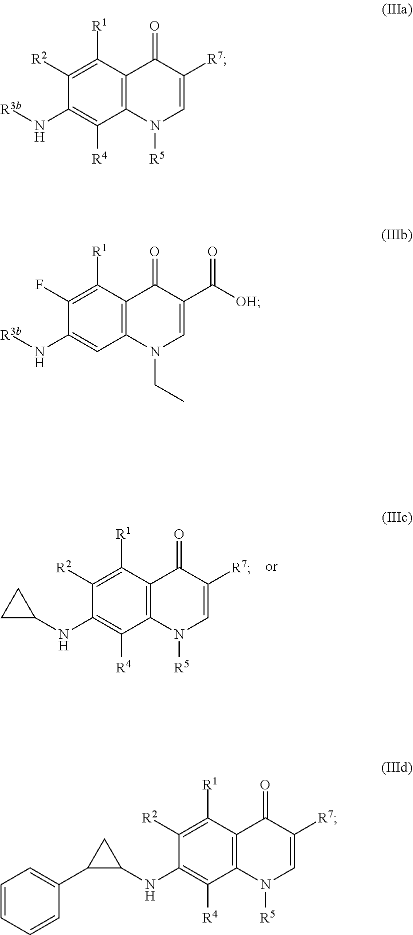
or a pharmaceutically acceptable salt or tautomer thereof, wherein R¹, R², R^{3b}, R⁴, R⁵ and R⁷ are as described herein.

79. The compound of any one of the preceding claims, wherein the compound is of Formulae (IVa), (IVb), (IVc), or (IVd): 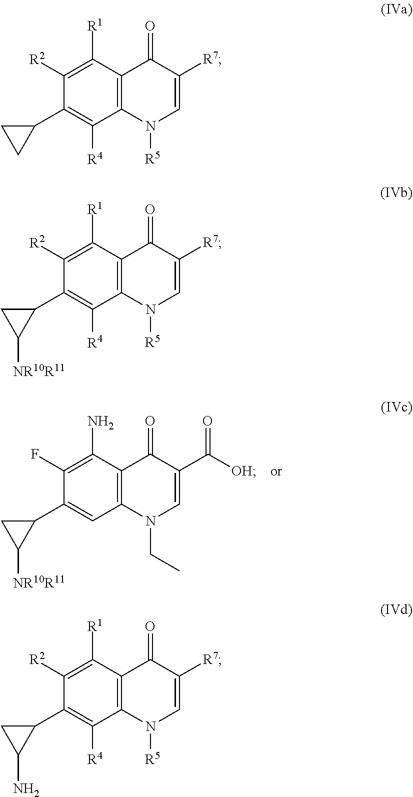
or a pharmaceutically acceptable salt or tautomer thereof, wherein R¹, R², R⁴, R⁵, R⁷, R¹⁰ and R¹¹ are as described herein.

80. The compound of any one of the preceding claims, wherein the compound is of Formulae (Va), (Vb), (Vc), or (Vd): 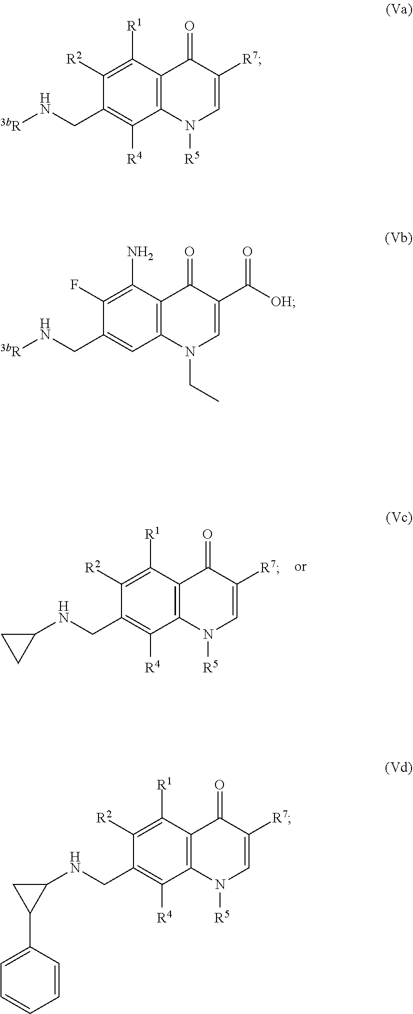
or a pharmaceutically acceptable salt or tautomer thereof, wherein R¹, R², R^{3b}, R⁴, R⁵ and R⁷ are as described herein.

81. The compound of any one of the preceding claims, wherein the compound is of Formulae (VIa), (VIb), (VIc), (VId), (VIe), (VIf), (VIg), (VIh), (VIi), (VIj), or (VIk): 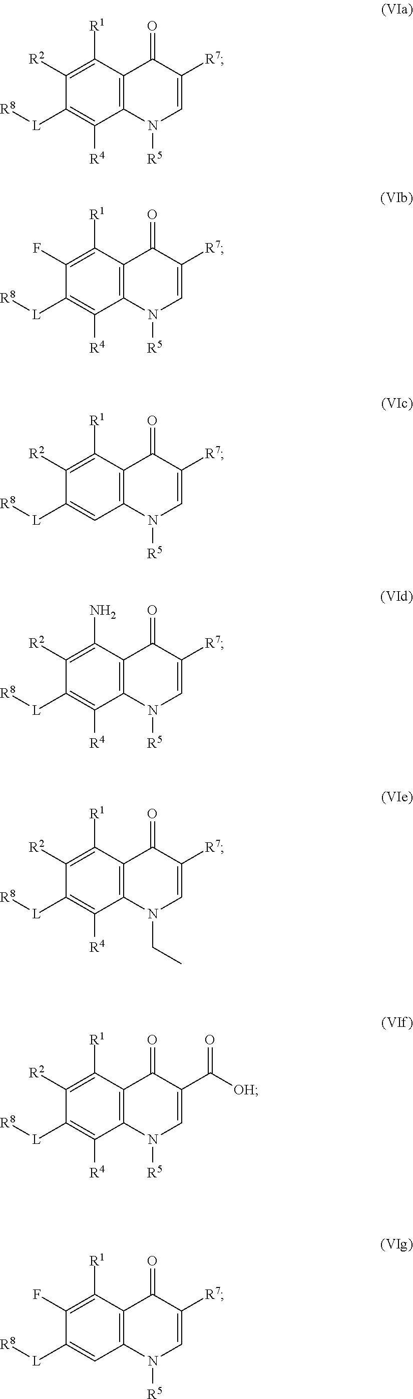 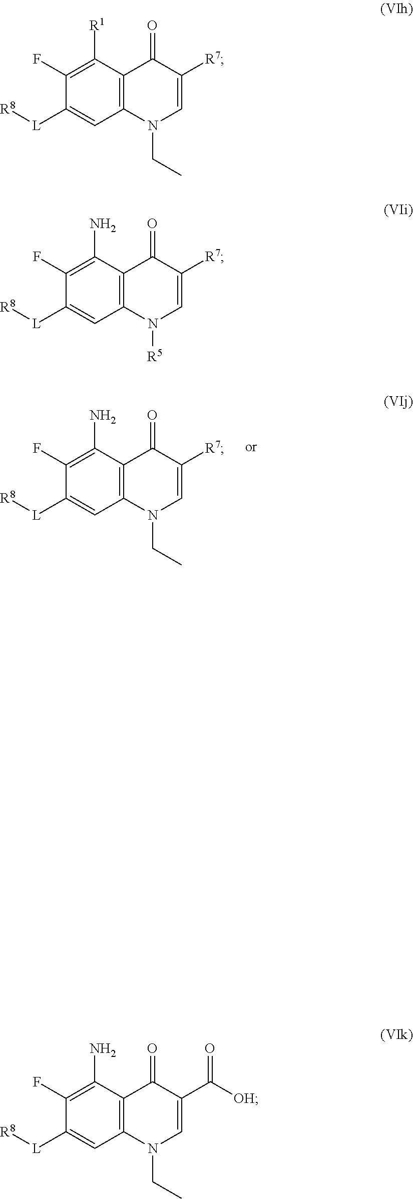
or a pharmaceutically acceptable salt or tautomer thereof, wherein R¹, R², R⁴, R⁵, R⁷, L and R⁸ are as described herein.

82. The compound of any one of the preceding claims, wherein the compound is of Formulae (VIIa) or (VIIb): 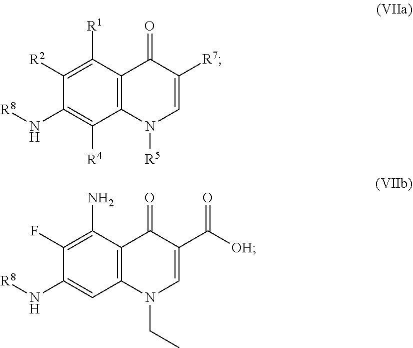
or a pharmaceutically acceptable salt or tautomer thereof, wherein R¹, R², R⁴, R⁵, R⁷ and R⁸ are as described herein.

83. The compound of any one of the preceding claims, wherein the compound is of Formulae (VIIIa) or (VIIIb): 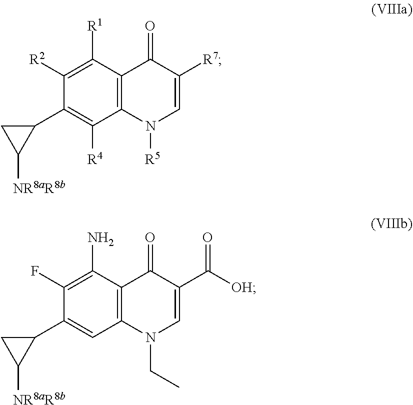
or a pharmaceutically acceptable salt or tautomer thereof, wherein R¹, R², R⁴, R⁵, R⁷, R^{8a} and R^{8b} are as described herein.

84. The compound of any one of the preceding claims, wherein the compound is of Formulae (IXa) or (IXb): 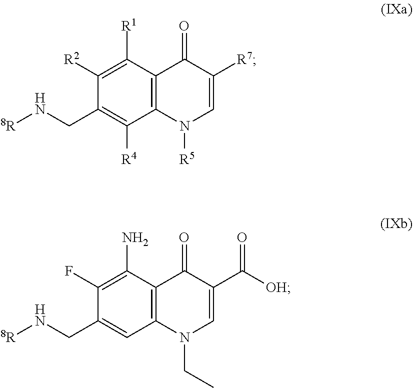
or a pharmaceutically acceptable salt or tautomer thereof, wherein R¹, R², R⁴, R⁵, R⁷ and R⁸ are as described herein.

85. A compound being selected from the group consisting of:

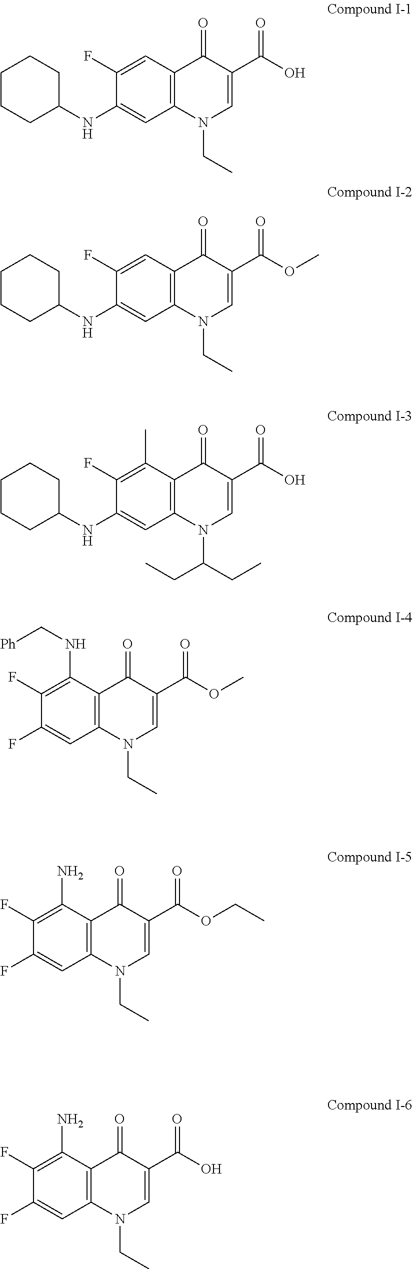 [SMILES: CCn1cc(C(=O)O)c(=O)c2cc(F)c(NC3CCCCC3)cc21 ; CCn1cc(C(=O)OC)c(=O)c2cc(F)c(NC3CCCCC3)cc21 ; CCC(CC)n1cc(C(=O)O)c(=O)c2c(C)c(F)c(NC3CCCCC3)cc21 ; CCn1cc(C(=O)OC)c(=O)c2c(NCc3ccccc3)c(F)c(F)cc21 ; CCOC(=O)c1cn(CC)c2cc(F)c(F)c(N)c2c1=O ; CCn1cc(C(=O)O)c(=O)c2c(N)c(F)c(F)cc21]

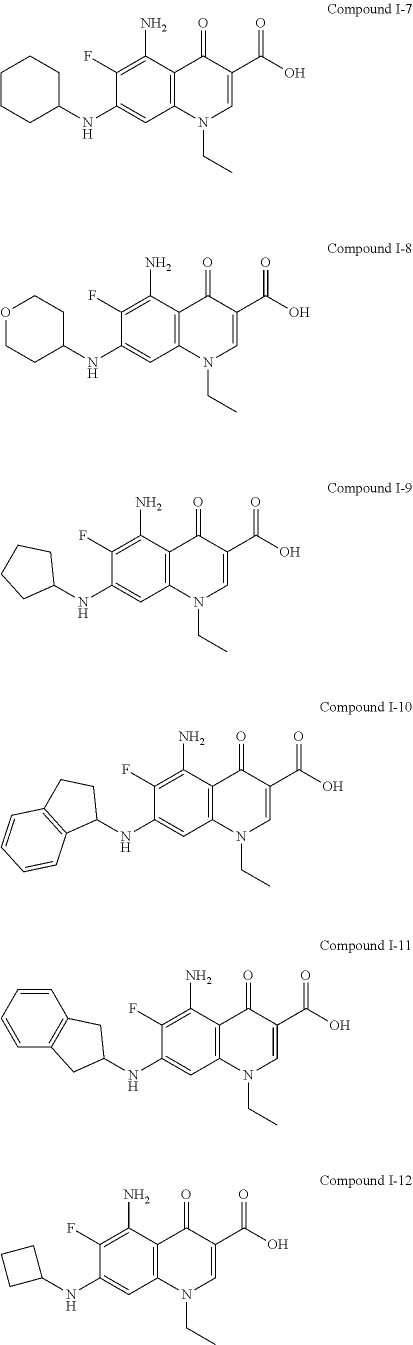 [SMILES: CCn1cc(C(=O)O)c(=O)c2c(N)c(F)c(NC3CCCCC3)cc21 ; CCn1cc(C(=O)O)c(=O)c2c(N)c(F)c(NC3CCOCC3)cc21 ; CCn1cc(C(=O)O)c(=O)c2c(N)c(F)c(NC3CCCC3)cc21 ; CCn1cc(C(=O)O)c(=O)c2c(N)c(F)c(NC3CCc4ccccc43)cc21 ; CCn1cc(C(=O)O)c(=O)c2c(N)c(F)c(NC3Cc4ccccc4C3)cc21 ; CCn1cc(C(=O)O)c(=O)c2c(N)c(F)c(NC3CCC3)cc21]

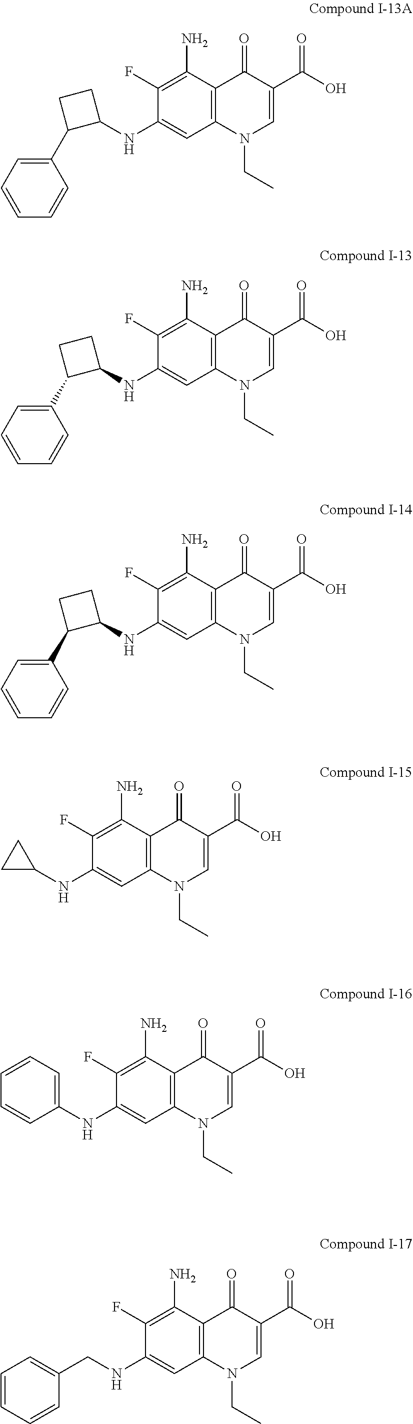 [SMILES: CCn1cc(C(=O)O)c(=O)c2c(N)c(F)c(NC3CCC3c3ccccc3)cc21 ; CCn1cc(C(=O)O)c(=O)c2c(N)c(F)c(N[C@@H]3CC[C@H]3c3ccccc3)cc21 ; CCn1cc(C(=O)O)c(=O)c2c(N)c(F)c(N[C@@H]3CC[C@@H]3c3ccccc3)cc21 ; CCn1cc(C(=O)O)c(=O)c2c(N)c(F)c(NC3CC3)cc21 ; CCn1cc(C(=O)O)c(=O)c2c(N)c(F)c(Nc3ccccc3)cc21 ; CCn1cc(C(=O)O)c(=O)c2c(N)c(F)c(NCc3ccccc3)cc21]

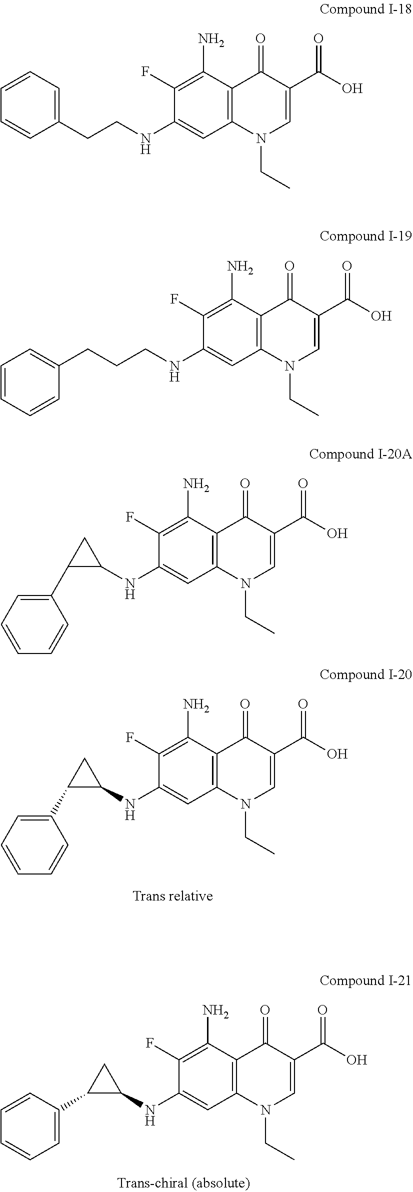 [SMILES: CCn1cc(C(=O)O)c(=O)c2c(N)c(F)c(NCCc3ccccc3)cc21 ; CCn1cc(C(=O)O)c(=O)c2c(N)c(F)c(NCCCc3ccccc3)cc21 ; CCn1cc(C(=O)O)c(=O)c2c(N)c(F)c(NC3CC3c3ccccc3)cc21 ; CCn1cc(C(=O)O)c(=O)c2c(N)c(F)c(N[C@@H]3C[C@H]3c3ccccc3)cc21 ; CCn1cc(C(=O)O)c(=O)c2c(N)c(F)c(N[C@@H]3C[C@H]3c3ccccc3)cc21]

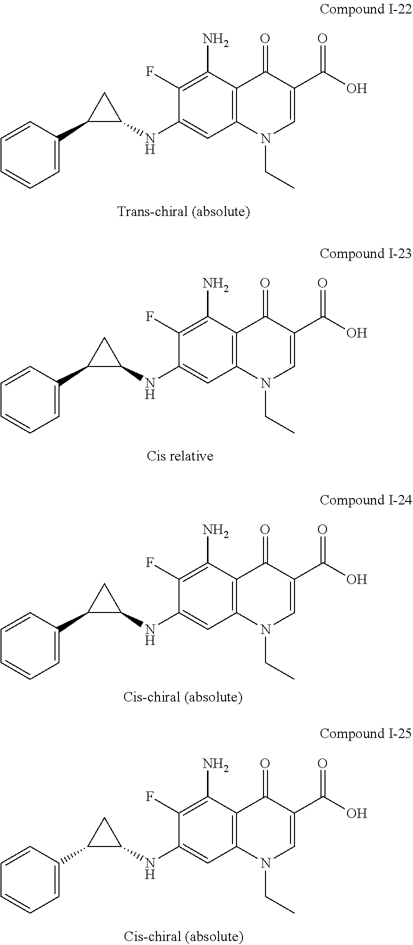 [SMILES: CCn1cc(C(=O)O)c(=O)c2c(N)c(F)c(N[C@H]3C[C@@H]3c3ccccc3)cc21 ; CCn1cc(C(=O)O)c(=O)c2c(N)c(F)c(N[C@@H]3C[C@@H]3c3ccccc3)cc21 ; CCn1cc(C(=O)O)c(=O)c2c(N)c(F)c(N[C@@H]3C[C@@H]3c3ccccc3)cc21 ; CCn1cc(C(=O)O)c(=O)c2c(N)c(F)c(N[C@H]3C[C@H]3c3ccccc3)cc21]

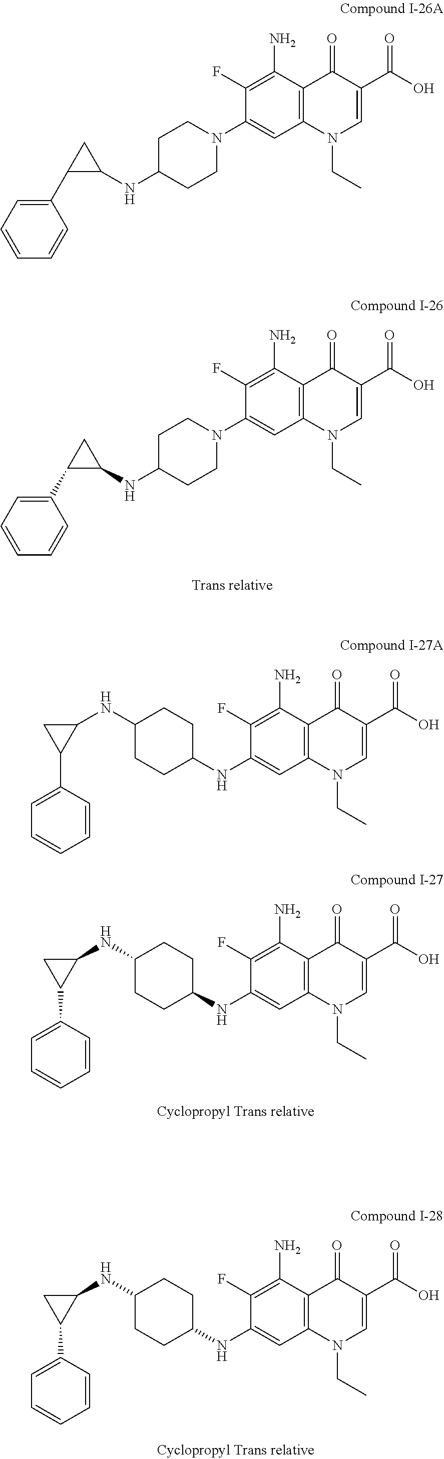

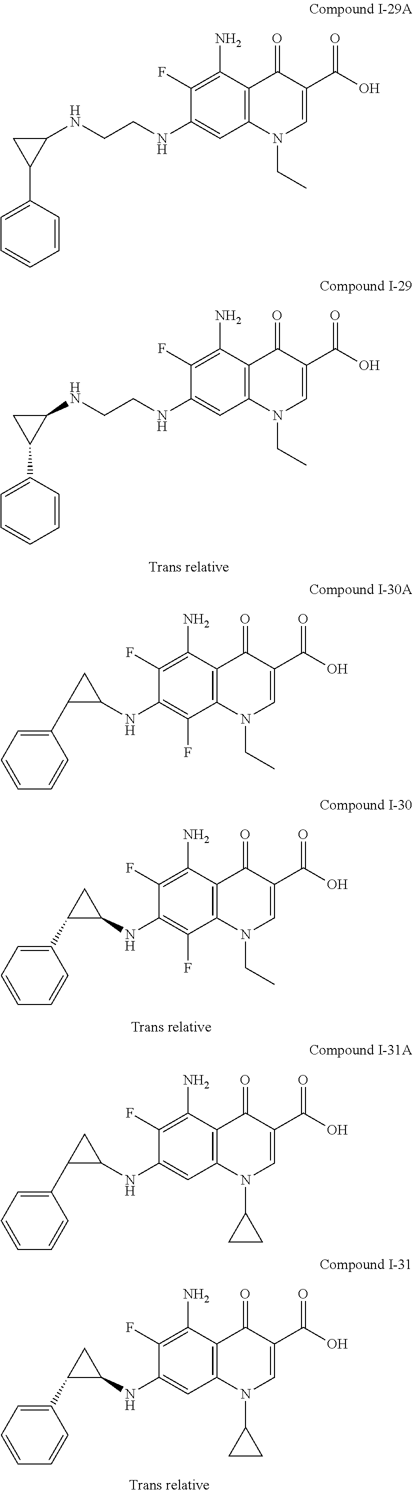 [SMILES: CCn1cc(C(=O)O)c(=O)c2c(N)c(F)c(NCCNC3CC3c3ccccc3)cc21 ; CCn1cc(C(=O)O)c(=O)c2c(N)c(F)c(NCCN[C@@H]3C[C@H]3c3ccccc3)cc21 ; CCn1cc(C(=O)O)c(=O)c2c(N)c(F)c(NC3CC3c3ccccc3)c(F)c21 ; CCn1cc(C(=O)O)c(=O)c2c(N)c(F)c(N[C@@H]3C[C@H]3c3ccccc3)c(F)c21 ; Nc1c(F)c(NC2CC2c2ccccc2)cc2c1c(=O)c(C(=O)O)cn2C1CC1 ; Nc1c(F)c(N[C@@H]2C[C@H]2c2ccccc2)cc2c1c(=O)c(C(=O)O)cn2C1CC1]

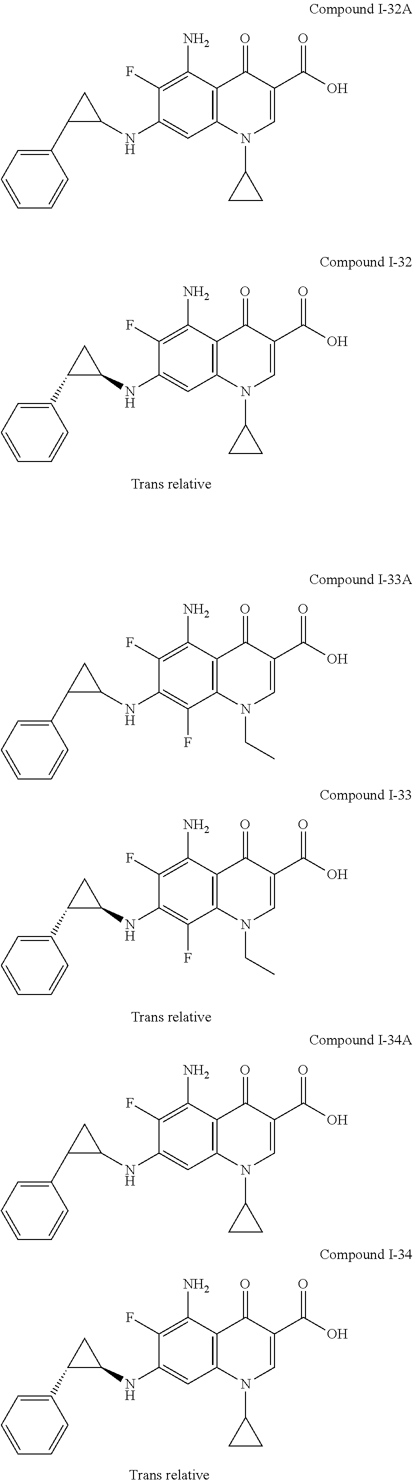 [SMILES: Nc1c(F)c(NC2CC2c2ccccc2)cc2c1c(=O)c(C(=O)O)cn2C1CC1 ; Nc1c(F)c(N[C@@H]2C[C@H]2c2ccccc2)cc2c1c(=O)c(C(=O)O)cn2C1CC1 ; CCn1cc(C(=O)O)c(=O)c2c(N)c(F)c(NC3CC3c3ccccc3)c(F)c21 ; CCn1cc(C(=O)O)c(=O)c2c(N)c(F)c(N[C@@H]3C[C@H]3c3ccccc3)c(F)c21 ; Nc1c(F)c(NC2CC2c2ccccc2)cc2c1c(=O)c(C(=O)O)cn2C1CC1 ; Nc1c(F)c(N[C@@H]2C[C@H]2c2ccccc2)cc2c1c(=O)c(C(=O)O)cn2C1CC1]

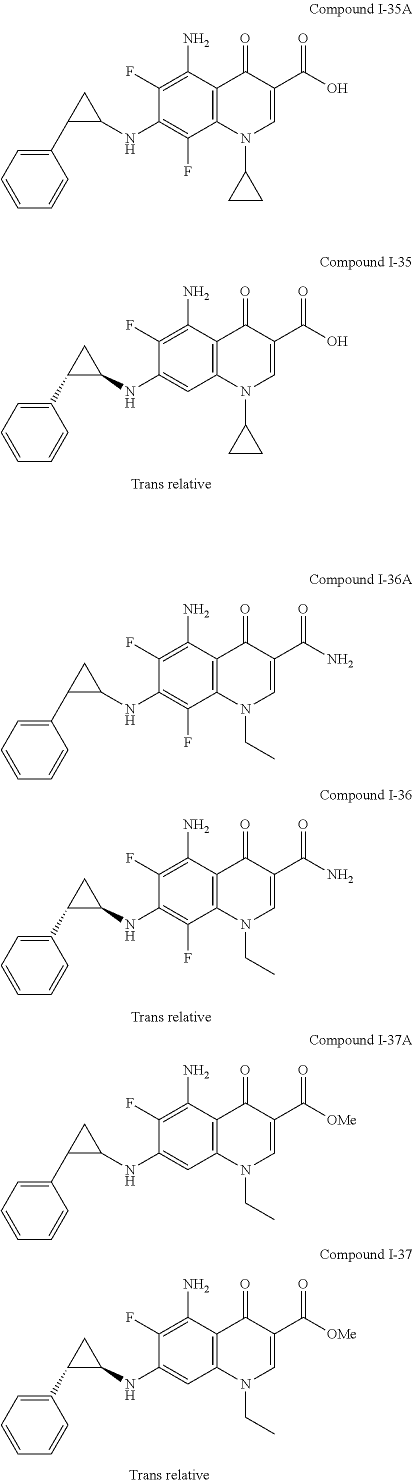 [SMILES: Nc1c(F)c(NC2CC2c2ccccc2)c(F)c2c1c(=O)c(C(=O)O)cn2C1CC1 ; Nc1c(F)c(N[C@@H]2C[C@H]2c2ccccc2)cc2c1c(=O)c(C(=O)O)cn2C1CC1 ; CCn1cc(C(N)=O)c(=O)c2c(N)c(F)c(NC3CC3c3ccccc3)c(F)c21 ; CCn1cc(C(N)=O)c(=O)c2c(N)c(F)c(N[C@@H]3C[C@H]3c3ccccc3)c(F)c21 ; CCn1cc(C(=O)OC)c(=O)c2c(N)c(F)c(NC3CC3c3ccccc3)cc21 ; CCn1cc(C(=O)OC)c(=O)c2c(N)c(F)c(N[C@@H]3C[C@H]3c3ccccc3)cc21]

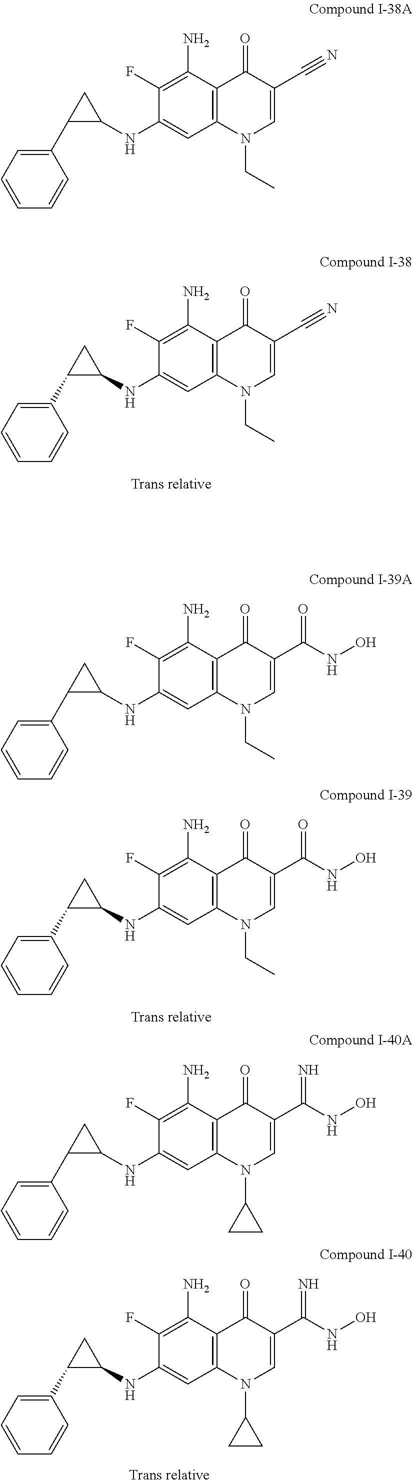 [SMILES: CCn1cc(C#N)c(=O)c2c(N)c(F)c(NC3CC3c3ccccc3)cc21 ; CCn1cc(C#N)c(=O)c2c(N)c(F)c(N[C@@H]3C[C@H]3c3ccccc3)cc21 ; CCn1cc(C(=O)NO)c(=O)c2c(N)c(F)c(NC3CC3c3ccccc3)cc21 ; CCn1cc(C(=O)NO)c(=O)c2c(N)c(F)c(N[C@@H]3C[C@H]3c3ccccc3)cc21 ; N=C(NO)c1cn(C2CC2)c2cc(NC3CC3c3ccccc3)c(F)c(N)c2c1=O ; N=C(NO)c1cn(C2CC2)c2cc(N[C@@H]3C[C@H]3c3ccccc3)c(F)c(N)c2c1=O]

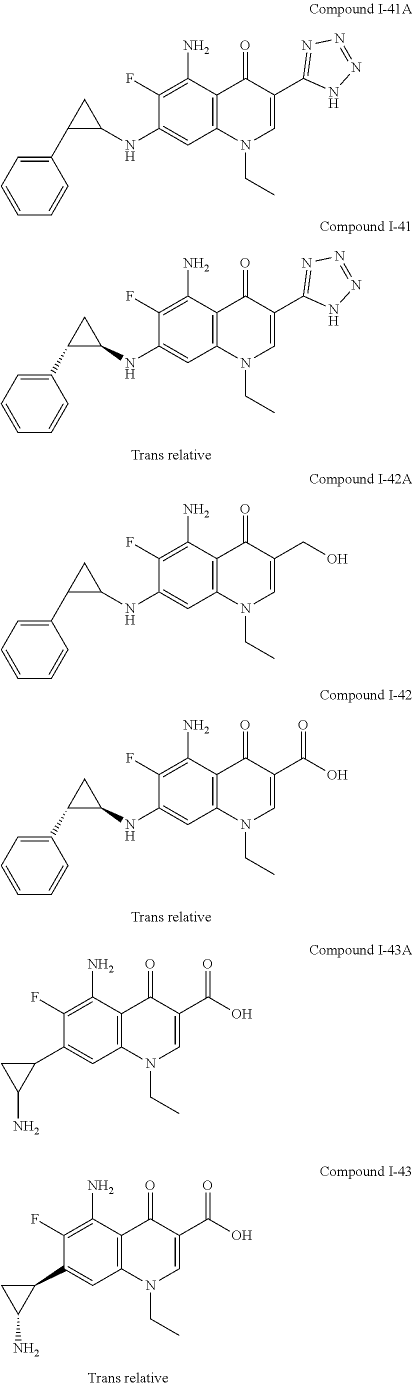 [SMILES: CCn1cc(-c2nnn[nH]2)c(=O)c2c(N)c(F)c(NC3CC3c3ccccc3)cc21 ; CCn1cc(-c2nnn[nH]2)c(=O)c2c(N)c(F)c(N[C@@H]3C[C@H]3c3ccccc3)cc21 ; CCn1cc(CO)c(=O)c2c(N)c(F)c(NC3CC3c3ccccc3)cc21 ; CCn1cc(C(=O)O)c(=O)c2c(N)c(F)c(N[C@@H]3C[C@H]3c3ccccc3)cc21 ; CCn1cc(C(=O)O)c(=O)c2c(N)c(F)c(C3CC3N)cc21 ; CCn1cc(C(=O)O)c(=O)c2c(N)c(F)c([C@@H]3C[C@H]3N)cc21]

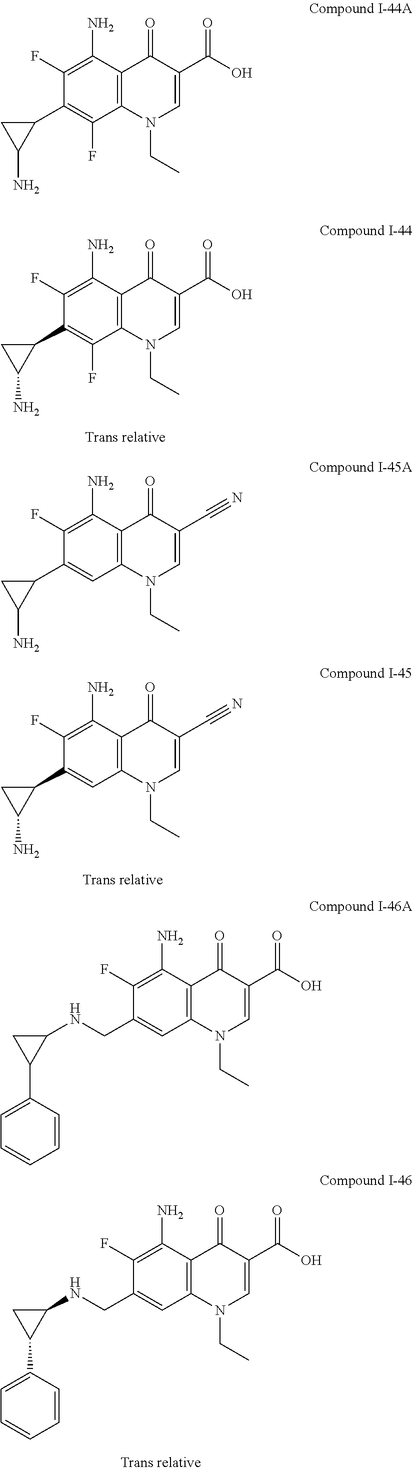 [SMILES: CCn1cc(C(=O)O)c(=O)c2c(N)c(F)c(C3CC3N)c(F)c21 ; CCn1cc(C(=O)O)c(=O)c2c(N)c(F)c([C@@H]3C[C@H]3N)c(F)c21 ; CCn1cc(C#N)c(=O)c2c(N)c(F)c(C3CC3N)cc21 ; CCn1cc(C#N)c(=O)c2c(N)c(F)c([C@@H]3C[C@H]3N)cc21 ; CCn1cc(C(=O)O)c(=O)c2c(N)c(F)c(CNC3CC3c3ccccc3)cc21 ; CCn1cc(C(=O)O)c(=O)c2c(N)c(F)c(CN[C@@H]3C[C@H]3c3ccccc3)cc21]

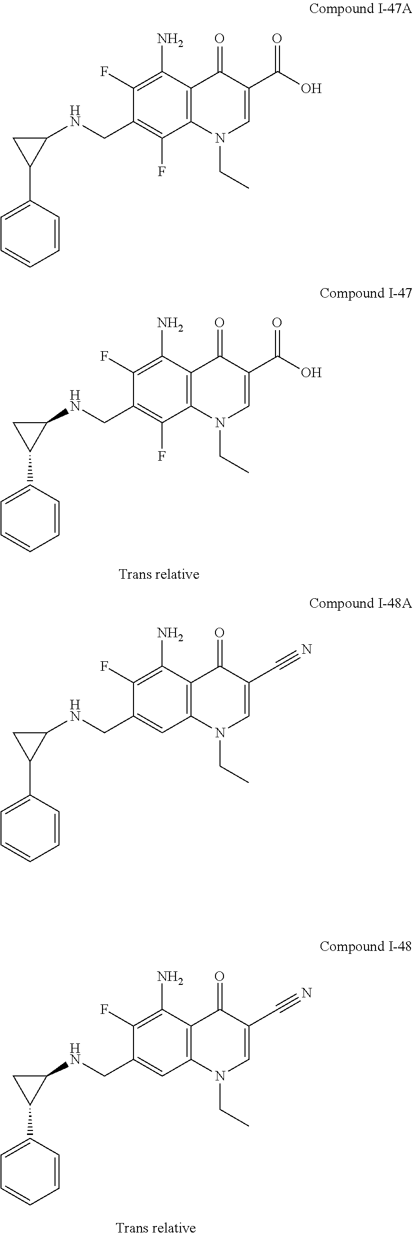 [SMILES: CCn1cc(C(=O)O)c(=O)c2c(N)c(F)c(CNC3CC3c3ccccc3)c(F)c21 ; CCn1cc(C(=O)O)c(=O)c2c(N)c(F)c(CN[C@@H]3C[C@H]3c3ccccc3)c(F)c21 ; CCn1cc(C#N)c(=O)c2c(N)c(F)c(CNC3CC3c3ccccc3)cc21 ; CCn1cc(C#N)c(=O)c2c(N)c(F)c(CN[C@@H]3C[C@H]3c3ccccc3)cc21]

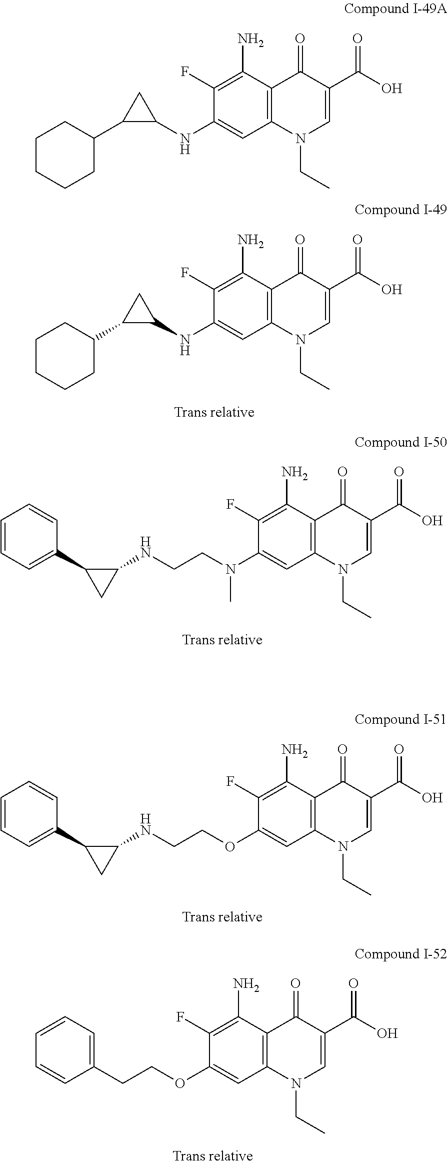

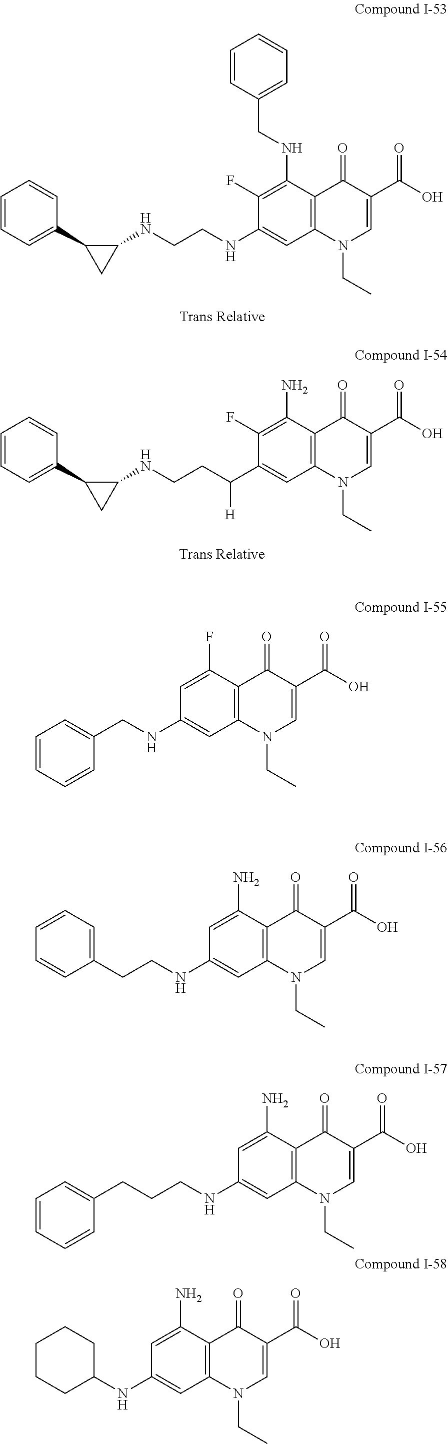 [SMILES: CCn1cc(C(=O)O)c(=O)c2c(NCc3ccccc3)c(F)c(NCCN[C@@H]3C[C@H]3c3ccccc3)cc21 ; CCn1cc(C(=O)O)c(=O)c2c(N)c(F)c(CCCN[C@@H]3C[C@H]3c3ccccc3)cc21 ; CCn1cc(C(=O)O)c(=O)c2c(F)cc(NCc3ccccc3)cc21 ; CCn1cc(C(=O)O)c(=O)c2c(N)cc(NCCc3ccccc3)cc21 ; CCn1cc(C(=O)O)c(=O)c2c(N)cc(NCCCc3ccccc3)cc21 ; CCn1cc(C(=O)O)c(=O)c2c(N)cc(NC3CCCCC3)cc21]

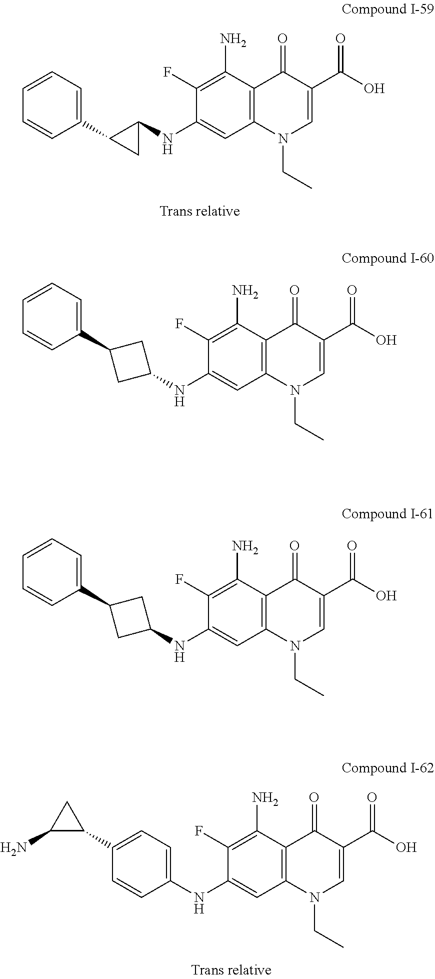 [SMILES: CCn1cc(C(=O)O)c(=O)c2c(N)c(F)c(N[C@H]3C[C@@H]3c3ccccc3)cc21 ; CCn1cc(C(=O)O)c(=O)c2c(N)c(F)c(N[C@H]3C[C@H](c4ccccc4)C3)cc21 ; CCn1cc(C(=O)O)c(=O)c2c(N)c(F)c(N[C@H]3C[C@@H](c4ccccc4)C3)cc21 ; CCn1cc(C(=O)O)c(=O)c2c(N)c(F)c(Nc3ccc([C@H]4C[C@@H]4N)cc3)cc21]

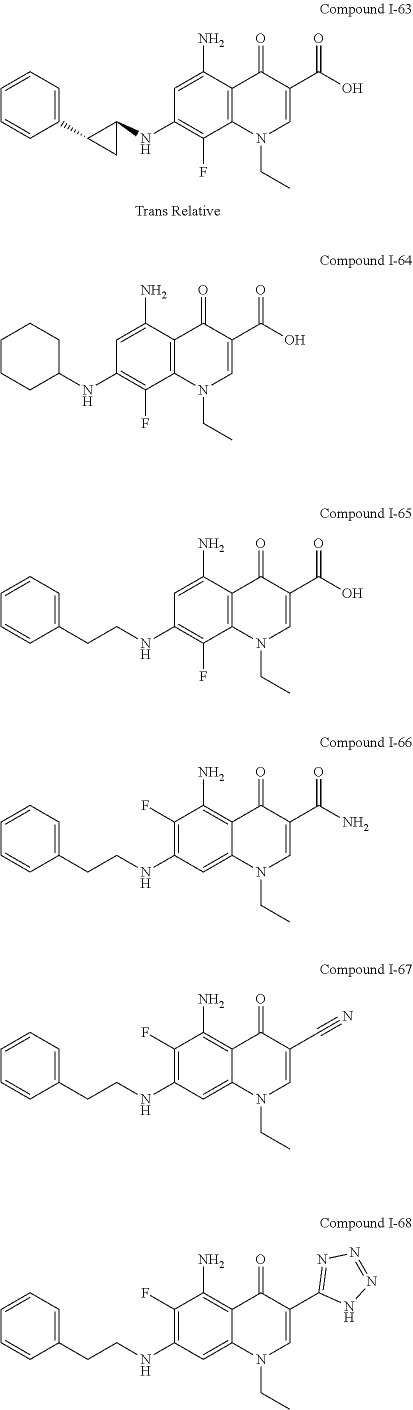 [SMILES: CCn1cc(C(=O)O)c(=O)c2c(N)cc(N[C@H]3C[C@@H]3c3ccccc3)c(F)c21 ; CCn1cc(C(=O)O)c(=O)c2c(N)cc(NC3CCCCC3)c(F)c21 ; CCn1cc(C(=O)O)c(=O)c2c(N)cc(NCCc3ccccc3)c(F)c21 ; CCn1cc(C(N)=O)c(=O)c2c(N)c(F)c(NCCc3ccccc3)cc21 ; CCn1cc(C#N)c(=O)c2c(N)c(F)c(NCCc3ccccc3)cc21 ; CCn1cc(-c2nnn[nH]2)c(=O)c2c(N)c(F)c(NCCc3ccccc3)cc21]

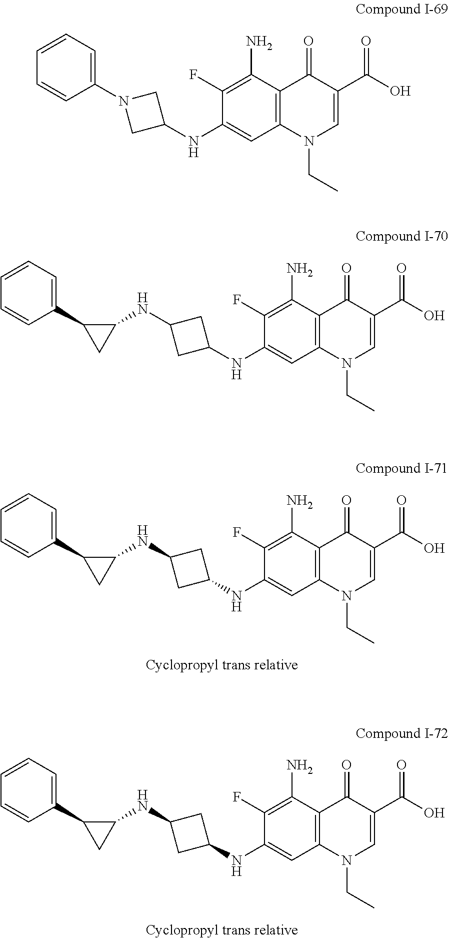 [SMILES: CCn1cc(C(=O)O)c(=O)c2c(N)c(F)c(NC3CN(c4ccccc4)C3)cc21 ; CCn1cc(C(=O)O)c(=O)c2c(N)c(F)c(NC3CC(N[C@@H]4C[C@H]4c4ccccc4)C3)cc21 ; CCn1cc(C(=O)O)c(=O)c2c(N)c(F)c(N[C@H]3C[C@H](N[C@@H]4C[C@H]4c4ccccc4)C3)cc21 ; CCn1cc(C(=O)O)c(=O)c2c(N)c(F)c(N[C@H]3C[C@@H](N[C@@H]4C[C@H]4c4ccccc4)C3)cc21]

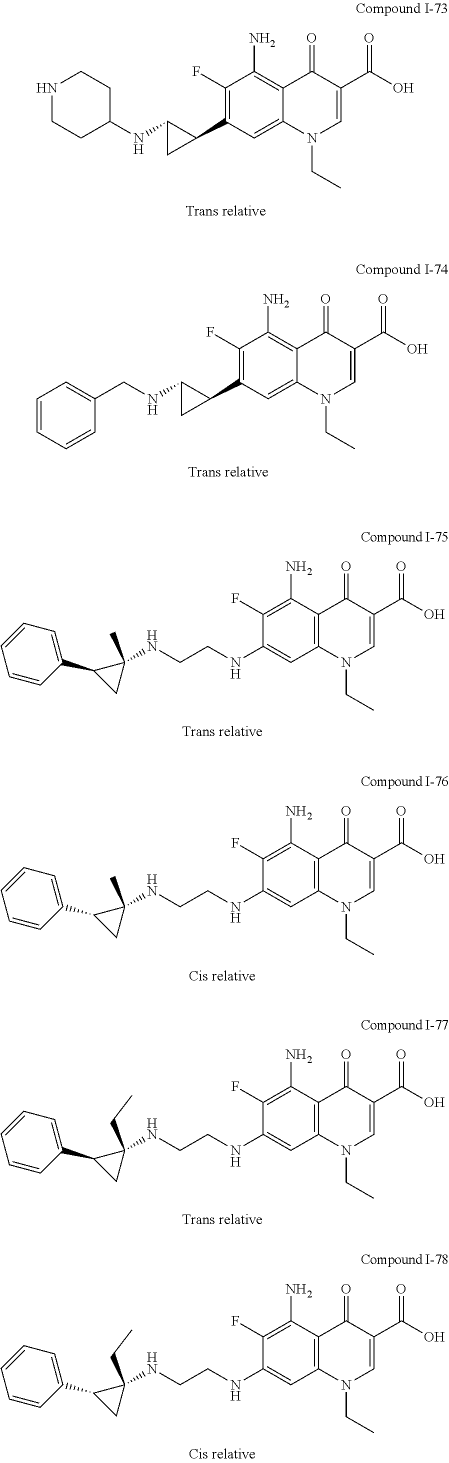 [SMILES: CCn1cc(C(=O)O)c(=O)c2c(N)c(F)c([C@H]3C[C@@H]3NC3CCNCC3)cc21 ; CCn1cc(C(=O)O)c(=O)c2c(N)c(F)c([C@H]3C[C@@H]3NCc3ccccc3)cc21 ; CCn1cc(C(=O)O)c(=O)c2c(N)c(F)c(NCCN[C@]3(C)C[C@H]3c3ccccc3)cc21 ; CCn1cc(C(=O)O)c(=O)c2c(N)c(F)c(NCCN[C@]3(C)C[C@@H]3c3ccccc3)cc21 ; CCn1cc(C(=O)O)c(=O)c2c(N)c(F)c(NCCN[C@]3(CC)C[C@H]3c3ccccc3)cc21 ; CCn1cc(C(=O)O)c(=O)c2c(N)c(F)c(NCCN[C@]3(CC)C[C@@H]3c3ccccc3)cc21]

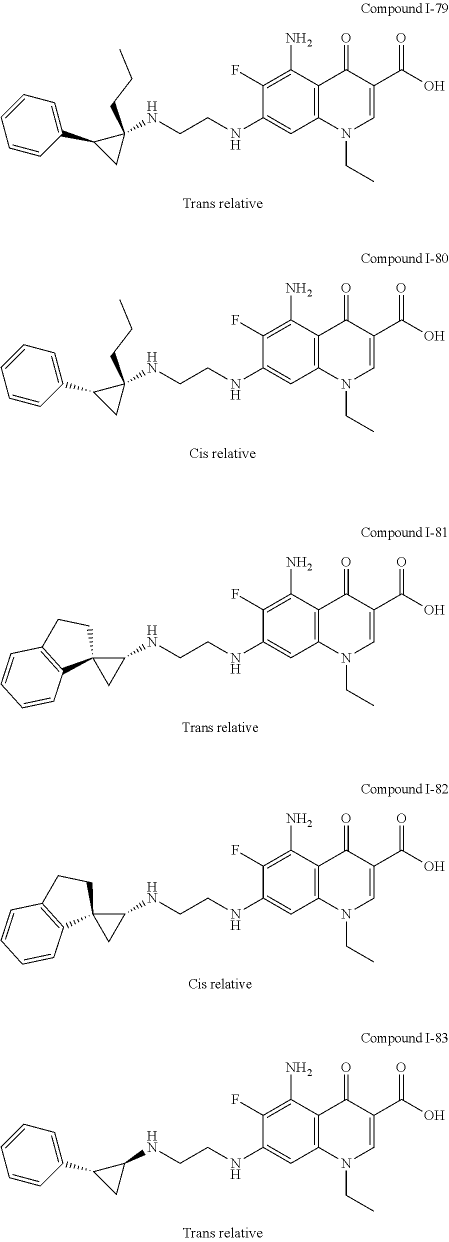 [SMILES: CCC[C@@]1(NCCNc2cc3c(c(N)c2F)c(=O)c(C(=O)O)cn3CC)C[C@H]1c1ccccc1 ; CCC[C@@]1(NCCNc2cc3c(c(N)c2F)c(=O)c(C(=O)O)cn3CC)C[C@@H]1c1ccccc1 ; CCn1cc(C(=O)O)c(=O)c2c(N)c(F)c(NCCN[C@@H]3C[C@]34CCc3ccccc34)cc21 ; CCn1cc(C(=O)O)c(=O)c2c(N)c(F)c(NCCN[C@@H]3C[C@@]34CCc3ccccc34)cc21 ; CCn1cc(C(=O)O)c(=O)c2c(N)c(F)c(NCCN[C@H]3C[C@@H]3c3ccccc3)cc21]

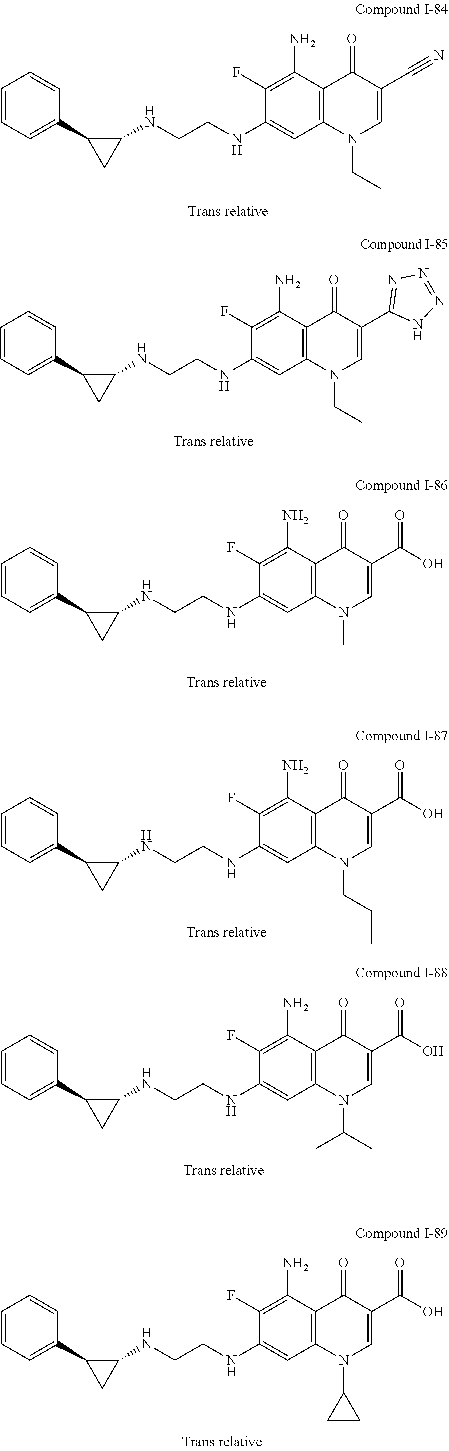 [SMILES: CCn1cc(C#N)c(=O)c2c(N)c(F)c(NCCN[C@@H]3C[C@H]3c3ccccc3)cc21 ; CCn1cc(-c2nnn[nH]2)c(=O)c2c(N)c(F)c(NCCN[C@@H]3C[C@H]3c3ccccc3)cc21 ; Cn1cc(C(=O)O)c(=O)c2c(N)c(F)c(NCCN[C@@H]3C[C@H]3c3ccccc3)cc21 ; CCCn1cc(C(=O)O)c(=O)c2c(N)c(F)c(NCCN[C@@H]3C[C@H]3c3ccccc3)cc21 ; CC(C)n1cc(C(=O)O)c(=O)c2c(N)c(F)c(NCCN[C@@H]3C[C@H]3c3ccccc3)cc21 ; Nc1c(F)c(NCCN[C@@H]2C[C@H]2c2ccccc2)cc2c1c(=O)c(C(=O)O)cn2C1CC1]

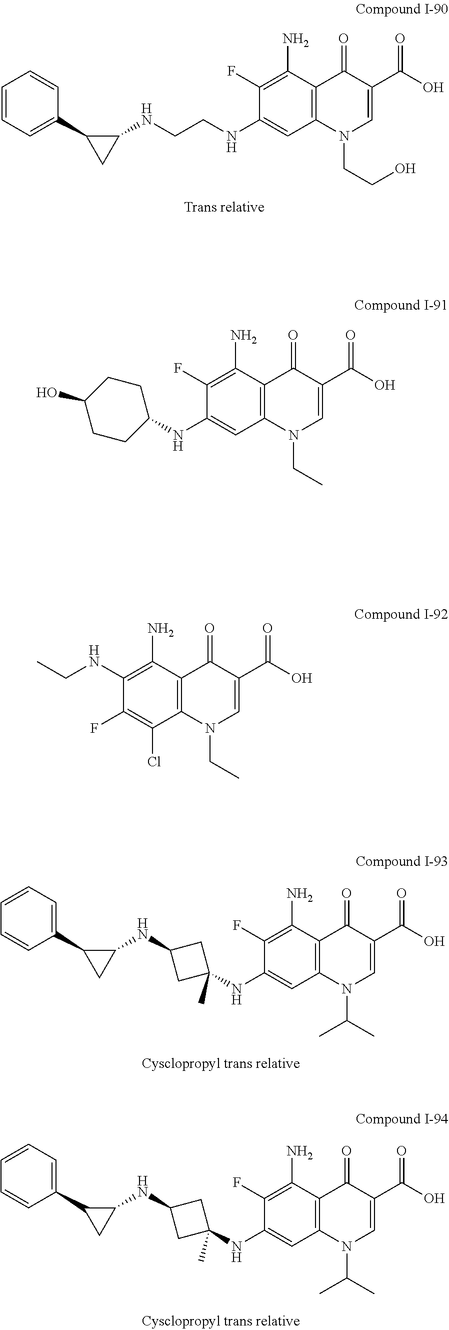

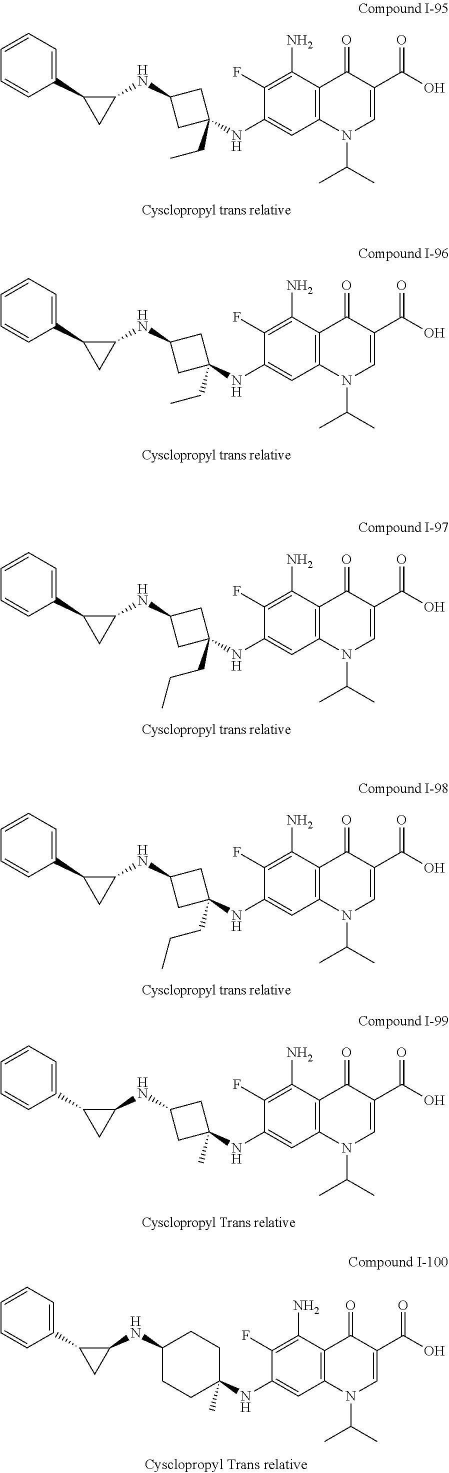

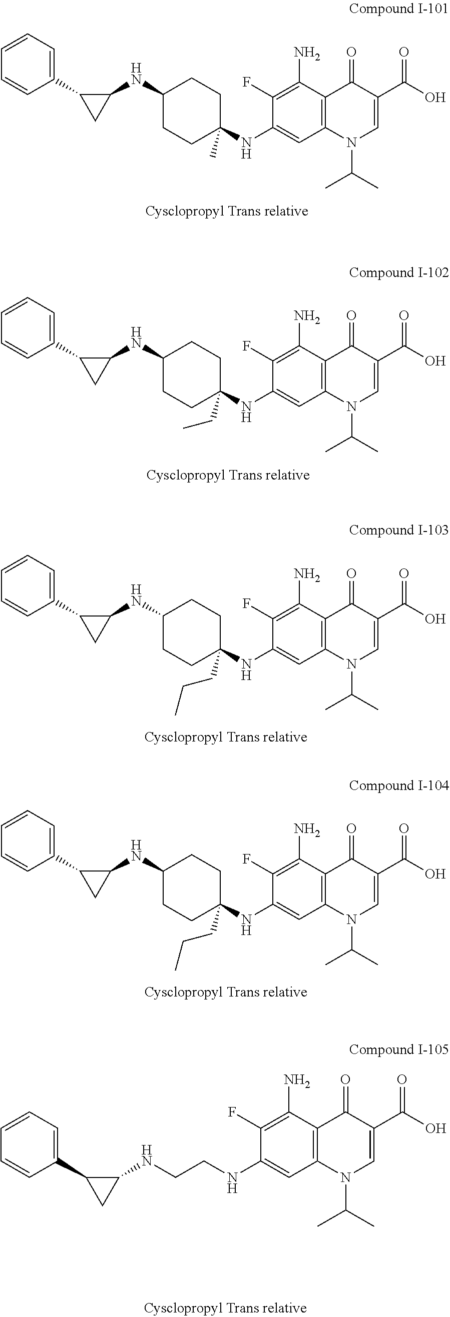 [SMILES: CC(C)n1cc(C(=O)O)c(=O)c2c(N)c(F)c(N[C@]3(C)CC[C@H](N[C@H]4C[C@@H]4c4ccccc4)CC3)cc21 ; CC[C@]1(Nc2cc3c(c(N)c2F)c(=O)c(C(=O)O)cn3C(C)C)CC[C@@H](N[C@H]2C[C@@H]2c2ccccc2)CC1 ; CCC[C@]1(Nc2cc3c(c(N)c2F)c(=O)c(C(=O)O)cn3C(C)C)CC[C@H](N[C@H]2C[C@@H]2c2ccccc2)CC1 ; CCC[C@]1(Nc2cc3c(c(N)c2F)c(=O)c(C(=O)O)cn3C(C)C)CC[C@@H](N[C@H]2C[C@@H]2c2ccccc2)CC1 ; CC(C)n1cc(C(=O)O)c(=O)c2c(N)c(F)c(NCCN[C@@H]3C[C@H]3c3ccccc3)cc21]

86. The compound of any one of the preceding claims, wherein the compound has a measured IC₅₀ value of about 0.1 nM to about 10 μM, about 0.5 nM to about 5 μM, about 1 nM to about 1 μM, about 2 nM to about 900 nM, about 3 nM to about 800 nM, about 4 nM to about 700 nM, about 5 nM to about 600 nM, about 10 nM to about 500 nM, about 20 nM to about 400 nM, about 30 nM to about 300 nM, about 40 nM to about 250 nM, about 50 nM to about 200 nM, about 60 nM to about 150 nM, about 70 nM to about 100 nM, or about 80 nM to about 90 nM against GSK3alpha and/or GSK3beta.

87. The compound of any one of the preceding claims, wherein the compound has a measured IC₅₀ value of about 0.1 nM or less, about 1 nM or less, about 2 nM or less, about 3 nM or less, about 4 nM or less, about 5 nM or less, about 10 nM or less, about 25 nM or less, about 50 nM or less, about 100 nM or less, about 250 nM or less, about 500 nM or less, about 1 μM or less, or about 10 μM or less against GSK3alpha and GSK3beta.

88. The compound of any one of the preceding claims, wherein the compound has a measured inhibitory activity value of about 0% to about 100%, about 1% to about 95%, about 2% to about 90%, about 3% to about 85%, about 4% to about 80%, about 5% to about 75%, about 10% to about 70%, about 15% to about 65%, about 20% to about 60%, about 25% to about 55%, about 30% to about 50%, about 35% to about 45%, or about 40% to about 45% against LSD-1 and/or LSD-2.

89. The compound of any one of the preceding claims, wherein the compound has a measured inhibitory activity value of about 1% or greater, about 2% or greater, about 3% or greater, about 4% or greater, about 5% or greater, about 10% or greater, about 15% or greater, about 20% or greater, about 25% or greater, about 30% or greater, about 40% or greater, about 50% or greater, about 60% or greater, about 70% or greater, about 80% or greater, about 90% or greater, or about 95% or greater against LSD-1 and LSD-2.

90. The compound of any one of the preceding claims, wherein the compound has a measured IC₅₀ value of about 0.1 nM to about 10 μM, about 0.5 nM to about 5 μM, about 1 nM to about 1 μM, about 2 nM to about 900 nM, about 3 nM to about 800 nM, about 4 nM to about 700 nM, about 5 nM to about 600 nM, about 10 nM to about 500 nM, about 20 nM to about 400 nM, about 30 nM to about 300 nM, about 40 nM to about 250 nM, about 50 nM to about 200 nM, about 60 nM to about 150 nM, about 70 nM to about 100 nM, or about 80 nM to about 90 nM against Foxo-1.

91. The compound of any one of the preceding claims, wherein the compound has a measured IC₅₀ value of about 0.1 nM or less, about 1 nM or less, about 2 nM or less, about 3 nM or less, about 4 nM or less, about 5 nM or less, about 10 nM or less, about 25 nM or less, about 50 nM or less, about 100 nM or less, about 250 nM or less, about 500 nM or less, about 1 μM or less, or about 10 μM or less against Foxo-1.

92. The compound of any one of the preceding claims, wherein the compound has a concentration effective to increase proliferation in an Lgr5 assay at about 0.1 nM to about 10 μM, about 0.5 nM to about 5 μM, about 1 nM to about 1 μM, about 2 nM to about 900 nM, about 3 nM to about 800 nM, about 4 nM to about 700 nM, about 5 nM to about 600 nM, about 10 nM to about 500 nM, about 20 nM to about 400 nM, about 30 nM to about 300 nM, about 40 nM to about 250 nM, about 50 nM to about 200 nM, about 60 nM to about 150 nM, about 70 nM to about 100 nM, or about 80 nM to about 90 nM.

93. The compound of any one of the preceding claims, wherein the compound has a measured IC₅₀ value of about 0.1 nM or less, about 1 nM or less, about 2 nM or less, about 3 nM or less, about 4 nM or less, about 5 nM or less, about 10 nM or less, about 25 nM or less, about 50 nM or less, about 100 nM or less, about 250 nM or less, about 500 nM or less, about 1 μM or less, or about 10 μM or less against Lgr5+.

94. The compound of any one of the preceding claims, wherein the compound has a measured Papp (B-A) coefficient of about 0.1 to about 50, about 0.25 to about 40, about 0.5 to about 35, about 0.75 to about 30, about 1 to about 25, about 2 to about 20, about 3 to about 15, about 4 to about 10, about 5 to about 9, about 6 to about 8, or about 6 to about 7.

95. The compound of any one of the preceding claims, wherein the compound has a measured Papp (B-A) coefficient of about 0.1 or greater, about 0.25 or greater, about 0.5 or greater, about 0.75 or greater, about 1 or greater, about 2 or greater, about 3 or greater, about 4 or greater, about 5 or greater, about 10 or greater, about 15 or greater, about 20 or greater, about 25 or greater, about 30 or greater, about 35 or greater, about 40 or greater, about 45 or greater, or about 50 or greater.

96. The compound of any one of the preceding claims, wherein the compound has a measured efflux ratio of about 0.1 to about 50, about 0.25 to about 40, about 0.5 to about 35, about 0.75 to about 30, about 1 to about 25, about 2 to about 20, about 3 to about 15, about 4 to about 10, about 5 to about 9, about 6 to about 8, or about 6 to about 7.

97. The compound of any one of the preceding claims, wherein the compound has a measured efflux ratio of about 50 or less, about 45 or less, about 40 or less, about 35 or less, about 30 or less, about 25 or less, about 20 or less, about 15 or less, about 10 or less, about 5 or less, about 4 or less, about 3 or less, about 2 or less, about 1 or less, about 0.75 or less, about 0.5 or less, about 0.2 or less, or about 0.1 or less.

98. The compound of any one of the preceding claims, wherein the compound has a measured concentration of about 0.001 μM to about 100 μM, about 0.002 μM to about 90 μM, about 0.005 μM to about 80 μM, about 0.01 μM to about 70 μM, about 0.05 μM to about 60 μM, about 0.1 μM to about 50 μM, about 0.5 μM to about 40 μM, about 1 μM to about 30 μM, about 2 μM to about 25 μM, about 3 μM to about 20 μM, about 4 μM to about 15 μM, about 5 μM to about 10 μM, about 6 μM to about 9 μM, or about 7 μM to about 8 μM in H₂O at pH 7.4.

99. The compound of any one of the preceding claims, wherein the compound has measured concentrations of about 0.001 μM or greater, about 0.002 μM or greater, about 0.005 μM or greater, about 0.01 μM or greater, about 0.05 μM or greater, about 0.1 μM or greater, about 0.5 μM or greater, about 1 μM or greater, about 2 μM or greater, about 3 μM or greater, 4 μM or greater, about 5 μM or greater, about 10 μM or greater, about 25 μM or greater, about 50 μM or greater, or about 100 μM or greater in H₂O at pH 7.4.

100. A pharmaceutical composition comprising a compound of any one of the preceding claims, or a pharmaceutically acceptable salt or tautomer thereof, and a pharmaceutically acceptable carrier.

101. The pharmaceutical composition of claim 100, further comprising an additional pharmaceutically active agent.

102. The pharmaceutical composition of any one of the preceding claims, further comprising at least one additional pharmaceutically active agent or a pharmaceutically acceptable salt or tautomer thereof.

103. The pharmaceutical composition of any one of the preceding claims, wherein the at least one additional pharmaceutically active agent is valproic acid or a pharmaceutically acceptable salt or tautomer thereof.

104. The pharmaceutical composition of any one of the preceding claims, wherein the at least one additional pharmaceutically active agent is tranylcypromine, or a pharmaceutically acceptable salt or tautomer thereof.

105. The pharmaceutical composition of any one of the preceding claims, wherein the at least one additional pharmaceutically active agent includes tranylcypromine, or a pharmaceutically acceptable salt or tautomer thereof, and valproic acid, or a pharmaceutically acceptable salt or tautomer thereof.

106. The pharmaceutical composition of any one of the preceding claims, wherein the pharmaceutically acceptable salt of valproic acid is sodium valproate.

107. The pharmaceutical composition of any one of the preceding claims, father comprising a poloxamer.

108. A method of expanding a population of cochlear cells in a cochlear tissue comprising a parent population, the method comprising contacting the cochlear tissue with a compound of any one of the preceding claims, or a pharmaceutically acceptable salt or tautomer thereof or a pharmaceutical composition of any one of the preceding claims.

109. The method of any one of the preceding claims, wherein the cochlear tissue is in a subject.

110. The method of any one of the preceding claims, wherein the contacting the cochlear tissue with the composition is achieved by administering the composition transtympanically to the subject.

111. The method of any one of the preceding claims, wherein contacting the cochlear tissue with the composition results in improved auditory functioning of the subject.

112. A method of generating tissue cells, the method comprising administering or causing to be administered to a stem cell population a compound of any one of the preceding claims, or a pharmaceutically acceptable salt or tautomer thereof or a pharmaceutical composition of any one of the preceding claims.

113. The method of any one of the preceding claims, wherein the tissue cells are cochlear cells.

114. The method of claim any one of the preceding claims, wherein the tissue cells are inner ear hair cells.

115. A method of treating or preventing a disease associated with absence or lack of certain tissue cells in a subject in need thereof, comprising administering or causing to be administered to a stem cell population a compound of any one of the preceding claims, or a pharmaceutically acceptable salt or tautomer thereof or a pharmaceutical composition of any one of the preceding claims.

116. The method of any one of the preceding claims, wherein the tissue cells are cochlear cells.

117. The method of any one of the preceding claims, wherein the tissue cells are inner ear hair cells.

118. A method of treating or preventing hearing loss in a subject in need thereof, the method comprising administering a compound of any one of the preceding claims, or a pharmaceutically acceptable salt or tautomer thereof or a pharmaceutical composition of any one of the preceding claims.

119. The method of any one of the preceding claims, wherein the hearing loss is sensorineural hearing loss.

120. The method of any one of the preceding claims, wherein the compound is administered transtympanically to the subject.

121. A method of facilitating the generation of inner ear hair cells, the method comprising: administering a compound of any one of the preceding claims or a pharmaceutically acceptable salt thereof, alone or in combination with an additional pharmaceutically active agent, to expand the stem cell population of cochlear tissue.

122. A method of regenerating or improving hearing in a mammal, the method comprising administering a compound of any one of the preceding claims, or a pharmaceutically acceptable salt or tautomer thereof, alone or in combination with an additional pharmaceutically active agent.

123. The method of any one of the preceding claims, wherein the administration is to a stem cell population to a subject.

124. A method of generating inner ear hair cells, the method comprising administering a compound of any one of the preceding claims, or a pharmaceutically acceptable salt or tautomer thereof, alone or in combination with an additional pharmaceutically active agent, wherein the method proliferates Lgr5⁺ cells in an initial population in vivo, resulting in an expanded population of Lgr5⁺ cells, resulting in generation of inner ear hair cells.

125. A method of facilitating generation of intestinal cells, the method comprising administering a compound of any one of the preceding claims or a pharmaceutically acceptable salt thereof, alone or in combination with an additional pharmaceutically active agent, to expand the stem cell population of intestinal epithelia.

126. The method of any one of the preceding claims, wherein the intestinal epithelia is regenerated.

127. The method of any one of the preceding claims, wherein the method is a treatment for promoting repair of damaged mucosa related to chemotherapy-induced gastrointestinal mucositis, graft-versus-host disease, gastric ulcer, Crohn's disease, or ulcerative colitis.

128. A method of expanding Lgr5⁺ cell population of intestinal epithelia, the method comprising: administering a compound of any one of the preceding claims or a pharmaceutically acceptable salt thereof, alone or in combination with an additional pharmaceutical agent.

129. A method of use of a compound of any one of the preceding claims, or a pharmaceutically acceptable salt or tautomer thereof, alone or in combination with an additional pharmaceutically active agent to regenerate Lgr5⁺ cell population intestinal cells in a mammal.

130. The method of any one of the preceding claims, wherein the method is a treatment for promoting the repair of damaged mucosa related to chemotherapy-induced gastrointestinal mucositis, Graft-versus-host disease, gastric ulcer, Crohn's disease, or ulcerative colitis.

131. A method of proliferating Lgr5⁺ epithelial cells in in vivo, the method comprising: administering a compound of any one of the preceding claims or a pharmaceutically acceptable salt thereof.

132. A method for expanding a population of vestibular cells in a vestibular tissue comprising contacting the vestibular tissue with (i) a compound of any one of the preceding claims or a pharmaceutically acceptable salt thereof, and (ii) an additional pharmaceutically active agent to form an expanded population of cells in the vestibular tissue.

133. A method of treating or preventing a vestibular disease, alopecia, oncology, acute myeloid leukemia, inflammation, Alzheimer's disease, Huntington's disease, Friedreick's ataxia, depression, anxiety, manic episodes of bipolar/mood disorders, Parkinson's, diabetes, bacterial infection, Anti-Trypanosoma brucei, ischemia, heart disease, vascular degeneration, and/or platelet aggregation in a subject in need thereof, the method comprising administering a compound of any one of the preceding claims or a pharmaceutically acceptable salt thereof, alone or in combination with an additional pharmaceutically active agent.

134. The method of any one of the preceding claims, wherein the compound of any one of the preceding claims functions as an inhibitor of at least one of FOXO-1, GSK3 α/β, and LSD-1.

135. The method of any one of the preceding claims, wherein the compound of any one of the preceding claims functions as an inhibitor of at least two of FOXO-1, GSK3 α/β, and LSD-1.

136. The method of any one of the preceding claims, wherein the compound of any one of the preceding claims functions as an inhibitor of FOXO-1, GSK3 α/β, and LSD-1.

137. A method of inhibiting LSD, GSK3, and/or FOXO in a cell, the method comprising contacting the cell with a compound of any one of the preceding claims or a pharmaceutically acceptable salt thereof.

138. The method of any one of the preceding claims, wherein the compound of any one of the preceding claims functions as a cell cycle progression pathway agonist.

139. The method of any one of the preceding claims, wherein the additional pharmaceutical agent is an HDAC inhibitor and/or a poloxamer.

140. The method of any one of the preceding claims, wherein the additional pharmaceutical agent includes at least one additional pharmaceutical agent.

141. The method of any one of the preceding claims, wherein the at least one additional pharmaceutical agent includes HDAC inhibitor and/or a poloxamer.

142. The method of any one of the preceding claims, wherein the HDAC inhibitor is valproic acid, or a pharmaceutically acceptable salt or tautomer thereof.

143. The method of any one of the preceding claims, wherein the at least one additional pharmaceutical agent includes tranylcypromine, or a pharmaceutically acceptable salt or tautomer thereof.

144. The method of any one of the preceding claims, wherein the at least one additional pharmaceutical agent includes valproic acid, or a pharmaceutically acceptable salt or tautomer thereof, and tranylcypromine, or a pharmaceutically acceptable salt or tautomer thereof.

145. The method of any one of the preceding claims, wherein the pharmaceutically acceptable salt of valproic acid is sodium valproate.

146. A system for treating or preventing a disease associated with absence or lack of certain tissue cells in a subject in need thereof, comprising administering:
a compound of any one of the preceding claims, or a pharmaceutically acceptable salt or tautomer thereof; and a trans-tympanic administrative device.

147. A method for proliferation of stem cells comprising administering to a cell population an effective amount of a compound of any one of the preceding claims.

148. The method of any one of the preceding claims, wherein the proliferation occurs in the absence of an additional activator or an additional inhibitor.

149. A compound of any one of the preceding claims, or a pharmaceutically acceptable salt or tautomer thereof, for use in treating or preventing a disease associated with absence or lack of certain tissue cells in a subject in need thereof.

150. A compound of any one of the preceding claims, or a pharmaceutically acceptable salt or tautomer thereof, for use in treating or preventing hearing loss in a subject in need thereof.

151. The compound, or a pharmaceutically acceptable salt or tautomer thereof, for use of any one of the preceding claims, wherein the hearing loss is sensorineural hearing loss.

152. Use of a compound of any one of the preceding claims, or a pharmaceutically acceptable salt or tautomer thereof, in the manufacture of a medicament for treating or preventing a disease associated with absence or lack of certain tissue cells in a subject in need thereof.

153. Use of a compound of any one of the preceding claims, or a pharmaceutically acceptable salt or tautomer thereof, in the manufacture of a medicament for treating or preventing hearing loss in a subject in need thereof.

154. The use of any one of the preceding claims, wherein the hearing loss is sensorineural hearing loss.

155. Use of a compound of any one of the preceding claims, or a pharmaceutically acceptable salt or tautomer thereof, in the manufacture of a medicament for treating or preventing a disease responding to LSD inhibition, GSK3 inhibition, and/or FOXO inhibition in a subject in need thereof.

156. Use of a compound of any one of the preceding claims, or a pharmaceutically acceptable salt or tautomer thereof, in the manufacture of a medicament for treating or preventing vestibular diseases, alopecia, oncology, Acute Myeloid Leukemia, inflammation, Alzheimer's disease, Huntington's disease, Friedreick's ataxia, depression, anxiety, manic episodes of bipolar/mood disorders, Parkinson's, diabetes, bacterial infection, Anti-Trypanosoma brucei, ischemia, heart disease, vascular degeneration, and/or platelet aggregation in a subject in need thereof.

157. The compound, the method, or the use of any one of the preceding claims, wherein the administration of the compound to a subject results into a higher Lgr5+ cell number in the subject, as compared to a comparable subject not being administered with the compound, by a factor ranging from about 2 fold to about 2,000,000 fold, from about 10 fold to about 1,000,000 fold, from about 100 fold to about 100,000 fold, or from about 1,000 fold to about 10,000 fold.

158. The compound, the method, or the use of any one of the preceding claims, wherein the administration of the compound to a subject results into a higher Lgr5+ cell number in the subject, as compared to a comparable subject not being administered with the compound, by a factor of greater than about 10 fold, greater than about 10,000 fold, greater than about 100,000 fold, or greater than about 1,000,000 fold.

159. The compound, the method, or the use of any one of the preceding claims, wherein the administration of the compound to a subject results into a higher Lgr5+ cell number in the subject, as compared to a comparable subject being administered with a Wnt agonist, by a factor ranging from about 0.1 fold to about 10 fold, from about 0.5 fold to about 5 fold, from about 1 fold to about 4 fold, or from about 1.5 fold to about 3 fold.

160. The compound, the method, or the use of any one of the preceding claims, wherein the administration of the compound to a subject results into a higher Lgr5+ cell number in the subject, as compared to a comparable subject being administered with a Wnt agonist, by a factor of greater than about 1.5 fold, greater than about 2 fold, greater than about 2.5 fold, greater than about 3 fold, greater than about 3.5 fold, or greater than about 4 fold.

161. The compound, the method, or the use of any one of the preceding claims, wherein the administration of the compound in combination with sodium valproate to a subject results into a higher percentage of Lgr5+ cells in the subject, as compared to a comparable subject being administered with the compound without sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

162. The compound, the method, or the use of any one of the preceding claims, wherein the administration of the compound in combination with sodium valproate to a subject results into a higher percentage of Lgr5+ cells in the subject, as compared to a comparable subject being administered with the compound without sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

163. The compound, the method, or the use of any one of the preceding claims, wherein the administration of the compound in combination with an LSD-1 inhibitor to a subject results into a higher percentage of Lgr5+ cells in the subject, as compared to a comparable subject being administered with the compound without the LSD-1 inhibitor, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

164. The compound, the method, or the use of any one of the preceding claims, wherein the administration of the compound in combination with an LSD-1 inhibitor to a subject results into a higher percentage of Lgr5+ cells in the subject, as compared to a comparable subject being administered with the compound without the LSD-1 inhibitor, by a factor of greater than about 1 fold, greater than about 1.5 fold, greater than about 2 fold, greater than about 2.5 fold, greater than about 3 fold, greater than about 3.5 fold, or greater than about 4 fold.

165. The compound, the method, or the use of any one of the preceding claims, wherein the administration of the compound in combination with sodium valproate and an LSD-1 inhibitor to a subject increases the percentage of Lgr5+ cells in the subject, as compared to a comparable subject being administered with the compound and sodium valproate without the LSD-1 inhibitor, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

166. The compound, the method, or the use of any one of the preceding claims, wherein the administration of the compound in combination with sodium valproate and an LSD-1 inhibitor to a subject increases the percentage of Lgr5+ cells in the subject, as compared to a comparable subject being administered with the compound and sodium valproate without the LSD-1 inhibitor, by a factor of greater than about 1 fold, greater than about 1.5 fold, greater than about 2 fold, greater than about 2.5 fold, greater than about 3 fold, greater than about 3.5 fold, or greater than about 4 fold.

167. The compound, the method, or the use of any one of the preceding claims, wherein the administration of the compound in combination with sodium valproate and an LSD-1 inhibitor to a subject increases the percentage of Lgr5+ cells in the subject, as compared to a comparable subject being administered with the compound and the LSD-1 inhibitor without sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

168. The compound, the method, or the use of any one of the preceding claims, wherein the administration of the compound in combination with sodium valproate and an LSD-1 inhibitor to a subject increases the percentage of Lgr5+ cells in the subject, as compared to a comparable subject being administered with the compound and the LSD-1 inhibitor without sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

169. The compound, the method, or the use of any one of the preceding claims, wherein the administration of the compound in combination with sodium valproate and an LSD-1 inhibitor to a subject increases the percentage of Lgr5+ cells in the subject, as compared to a comparable subject being administered with the compound without the LSD-1 inhibitor or sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

170. The compound, the method, or the use of any one of the preceding claims, wherein the administration of the compound in combination with sodium valproate and an LSD-1 inhibitor to a subject increases the percentage of Lgr5+ cells in the subject, as compared to a comparable subject being administered with the compound without the LSD-1 inhibitor or sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

171. The compound, the method, or the use of any one of the preceding claims, wherein when a cell population is contacted with the compound, the contacting results into a higher Lgr5+ cell number in the cell population, as compared to a comparable cell population not being contacted with the compound, by a factor ranging from about 2 fold to about 2,000,000 fold, from about 10 fold to about 1,000,000 fold, from about 100 fold to about 100,000 fold, or from about 1,000 fold to about 10,000 fold.

172. The compound, the method, or the use of any one of the preceding claims, wherein when a cell population is contacted with the compound, the contacting results into a higher Lgr5+ cell number in the cell population, as compared to a comparable cell population not being contacted with the compound, by a factor of greater than about 10 fold, greater than about 10,000 fold, greater than about 100,000 fold, or greater than about 1,000,000 fold.

173. The compound, the method, or the use of any one of the preceding claims, wherein when a cell population is contacted with the compound, the contacting results into a higher Lgr5+ cell number in the cell population, as compared to a comparable cell population being contacted with a Wnt agonist, by a factor ranging from about 0.1 fold to about 10 fold, from about 0.5 fold to about 5 fold, from about 1 fold to about 4 fold, or from about 1.5 fold to about 3 fold.

174. The compound, the method, or the use of any one of the preceding claims, wherein when a cell population is contacted with the compound, the contacting results into a higher Lgr5+ cell number in the cell population, as compared to a comparable cell population being contacted with a Wnt agonist, by a factor of greater than about 1.5 fold, greater than about 2 fold, greater than about 2.5 fold, greater than about 3 fold, greater than about 3.5 fold, or greater than about 4 fold.

175. The compound, the method, or the use of any one of the preceding claims, wherein when a cell population is contacted with the compound in combination with sodium valproate, the contacting results into a higher percentage of Lgr5+ cells in the cell population, as compared to a comparable cell population being contacted with the compound without sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

176. The compound, the method, or the use of any one of the preceding claims, wherein when a cell population is contacted with the compound in combination with sodium valproate, the contacting results into a higher percentage of Lgr5+ cells in the cell population, as compared to a comparable cell population being contacted with the compound without sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

177. The compound, the method, or the use of any one of the preceding claims, wherein when a cell population is contacted with the compound in combination with an LSD-1 inhibitor, the contacting results into a higher percentage of Lgr5+ cells in the cell population, as compared to a comparable cell population being contacted with the compound without the LSD-1 inhibitor, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

178. The compound, the method, or the use of any one of the preceding claims, wherein when a cell population is contacted with the compound in combination with an LSD-1 inhibitor, the contacting results into a higher percentage of Lgr5+ cells in the cell population, as compared to a comparable cell population being contacted with the compound without the LSD-1 inhibitor, by a factor of greater than about 1 fold, greater than about 1.5 fold, greater than about 2 fold, greater than about 2.5 fold, greater than about 3 fold, greater than about 3.5 fold, or greater than about 4 fold.

179. The compound, the method, or the use of any one of the preceding claims, wherein when a cell population is contacted with the compound in combination with sodium valproate and an LSD-1 inhibitor, the contacting increases the percentage of Lgr5+ cells in the cell population, as compared to a comparable cell population being contacted with the compound and sodium valproate without the LSD-1 inhibitor, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

180. The compound, the method, or the use of any one of the preceding claims, wherein when a cell population is contacted with the compound in combination with sodium valproate and an LSD-1 inhibitor, the contacting increases the percentage of Lgr5+ cells in the cell population, as compared to a comparable cell population being contacted with the compound and sodium valproate without the LSD-1 inhibitor, by a factor of greater than about 1 fold, greater than about 1.5 fold, greater than about 2 fold, greater than about 2.5 fold, greater than about 3 fold, greater than about 3.5 fold, or greater than about 4 fold.

181. The compound, the method, or the use of any one of the preceding claims, wherein when a cell population is contacted with the compound in combination with sodium valproate and an LSD-1 inhibitor, the contacting increases the percentage of Lgr5+ cells in the cell population, as compared to a comparable cell population being contacted with the compound and the LSD-1 inhibitor without sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

182. The compound, the method, or the use of any one of the preceding claims, wherein when a cell population is contacted with the compound in combination with sodium valproate and an LSD-1 inhibitor, the contacting increases the percentage of Lgr5+ cells in the cell population, as compared to a comparable cell population being contacted with the compound and the LSD-1 inhibitor without sodium valproate, by a factor of greater than about 1 fold, greater than about 1.5 fold, greater than about 2 fold, greater than about 2.5 fold, greater than about 3 fold, greater than about 3.5 fold, or greater than about 4 fold.

183. The compound, the method, or the use of any one of the preceding claims, wherein when a cell population is contacted with the compound in combination with sodium valproate and an LSD-1 inhibitor, the contacting increases the percentage of Lgr5+ cells in the cell population, as compared to a comparable cell population being contacted with the compound without the LSD-1 inhibitor or sodium valproate, by a factor ranging from about 1 fold to about 10 fold, from about 1.1 fold to about 5 fold, from about 1.2 fold to about 4 fold, from about 1.3 fold to about 3 fold, or from about 1.5 fold to about 2.5 fold.

184. The compound, the method, or the use of any one of the preceding claims, wherein when a cell population is contacted with the compound in combination with sodium valproate and an LSD-1 inhibitor, the contacting increases the percentage of Lgr5+ cells in the cell population, as compared to a comparable cell population being contacted with the compound without the LSD-1 inhibitor or sodium valproate, by a factor of greater than about 1 fold, greater than about 1.5 fold, greater than about 2 fold, greater than about 2.5 fold, greater than about 3 fold, greater than about 3.5 fold, or greater than about 4 fold.
